SEQUENCE LISTING (10 sequences)

SEQ ID NO: 1 - protein, 26 aa, synthetic construct
  REGION 1..26: note = Secretory sequence
MRVMAPRTLILLLSGALALTETWAGS

SEQ ID NO: 2 - protein, 55 aa, synthetic construct
  REGION 1..55: note = MITD
IVGIVAGLAVLAVVVIGAVVATVMCRRKSSGGKGGSYSQAASSDSAQGSDVSLTA

SEQ ID NO: 3 - protein, 65 aa, synthetic construct
  REGION 1..65: note = P2P16
KKQYIKANSKFIGITELKKLGGGKRGGGKKMTNSVDDALINSTKIYSYFPSVISKVNQGAQGKKL

SEQ ID NO: 4 - protein, 10 aa, synthetic construct
  REGION 1..10: note = Linker
GGSGGGGSGG

SEQ ID NO: 5 - protein, 14 aa, synthetic construct
  REGION 1..14: note = Linker
GSSGGGGSPGGGSS

SEQ ID NO: 6 - RNA, 47 nt, synthetic construct
  misc_feature 1..47: note = 5'-UTR
  source 1..47: mol_type = other RNA
aactagtattcttctggtccccacagactcagagagaacccgccacc

SEQ ID NO: 7 - RNA, 278 nt, synthetic construct
  misc_feature 1..278: note = 3'-UTR
  source 1..278: mol_type = other RNA
ctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacacc

SEQ ID NO: 8 - RNA, 110 nt, synthetic construct
  misc_feature 1..110: note = A30L70
  source 1..110: mol_type = other RNA
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 9 - protein, 15 aa, synthetic construct
  REGION 1..15: note = P2
QYIKANSKFIGITEL

SEQ ID NO: 10 - protein, 32 aa, synthetic construct
  REGION 1..32: note = P16
MTNSVDDALINSTKIYSYFPSVISKVNQGAQG

(19) United States
(12) Patent Application Publication
(10) Pub. No.: US 2026/0021199 A1
(43) Pub. Date: Jan. 22, 2026

(54) RNA LIPID NANOPARTICLES (LNPs) COMPRISING A POLYOXAZOLINE AND/OR POLYOXAZINE POLYMER

(71) Applicant: BIONTECH SE, Mainz (DE)

(72) Inventors: Heinrich HAAS, Mainz (DE); Jorge MORENO HERRERO, Mainz (DE); Fabian LINK, Mainz (DE); Benjamin WEBER, Mainz (DE); Victor RETAMERO DE LA ROSA, Gent (BE); Rosa VILLANOVA, Gent (BE); Ehsan MEHRAVAR, Mainz (DE); Hossam HEFESHA, Mainz (DE)

(21) Appl. No.: 18/842,589

(22) PCT Filed: Mar. 1, 2023
(86) PCT No.: PCT/EP2023/055244

§ 371 Date: Aug. 29, 2024

(30) Foreign Application Priority Data
Mar. 1, 2022 (WO) PCT/EP2022/055122

(51) Int. Cl. A61K 48/00; C12N 15/88

(52) U.S. Cl. CPC A61K 48/0041; A61K 48/0058; C12N 15/88

(57) ABSTRACT

The present disclosure relates to RNA nanoparticles (LNPs) for delivery of RNA to target tissues after administration, in particular after parenteral administration such as intravenous, intramuscular, subcutaneous, intratumoral, intraarterial, intradermal, dermal, intranasal, rectal or oral administration, and compositions comprising such RNA LNPs. The RNA LNPs in some embodiments comprise single-stranded RNA such as mRNA which encodes a peptide or protein of interest, such as a pharmaceutically active peptide or protein. The RNA is taken up by cells of a target tissue and the RNA is translated into the encoded peptide or protein, which may exhibit its physiological activity. Furthermore, the present disclosure relates to certain conjugates of (a) a polyoxazoline (POX) and/or polyoxazine (POZ) polymer and (b) one or more hydrophobic chains, compositions comprising such conjugates, and the uses of such conjugates and compositions.

### TECHNICAL FIELD

The present disclosure relates to RNA nanoparticles (LNPs) for delivery of RNA to target tissues after administration, in particular after parenteral administration such as intravenous, intramuscular, subcutaneous, intratumoral, intraarterial, intradermal, dermal, intranasal, rectal or oral administration, and compositions comprising such RNA LNPs. The RNA LNPs in some embodiments comprise single-stranded RNA such as mRNA which encodes a peptide or protein of interest, such as a pharmaceutically active peptide or protein. The RNA is taken up by cells of a target tissue and the RNA is translated into the encoded peptide or protein, which may exhibit its physiological activity. Furthermore, the present disclosure relates to certain conjugates of (a) a polyoxazoline (POX) and/or polyoxazine (POZ) polymer and (b) one or more hydrophobic chains, compositions comprising such conjugates, and the uses of such conjugates and compositions.

### BACKGROUND

The use of RNA for delivery of foreign genetic information into target cells offers an attractive alternative to DNA. The advantages of using RNA include transient expression and a non-transforming character. RNA does not need to enter the nucleus in order to be expressed and moreover cannot integrate into the host genome, thereby eliminating the risk of oncogenesis.

RNA may be delivered to a subject using different delivery vehicles, mostly based on cationic polymers or lipids which together with the RNA form nanoparticles. The nanoparticles are intended to protect the RNA from degradation, enable delivery of the RNA to the target site and facilitate cellular uptake and processing by the target cells. For delivery efficacy, in addition to the molecular composition, parameters like particle size, charge, or grafting with molecular moieties, such as polyethylene glycol (PEG) or ligands, play a role. Grafting with PEG is considered to reduce serum interactions, to increase serum stability and to increase circulation time, which can be helpful for certain targeting approaches. Ligands which bind to receptors at the target site can help to improve targeting efficacy. Furthermore, PEGylation can be used for particle engineering. For example, if LNPs are manufactured by mixing an aqueous phase of the RNA with an organic phase of the lipids a certain fraction of PEG-conjugated lipid in the lipid mixture is required, otherwise the particles aggregate during the mixing step. It has been shown that by variation of the molar fraction of PEG-lipids comprising PEG at different molar masses the size of the particles can be adjusted. As well, the particle size may be adjusted by variation of the molar mass of the PEG moiety of the PEGylated lipids. Typical sizes which are accessible are in the range between 30 and 200 nm (Belliveau et al., 2012, Molecular Therapy-Nucleic Acids 1, e37). So-formed particles have additionally the advantage, that, due to the PEG fraction, they interact less with serum components, and have a longer circulation half-life, which is desirable in many drug delivery approaches. Without PEG-lipids, no particles with discrete size can be formed; the particles form large aggregates and precipitate.

So, for techniques where LNPs are formed from an ethanolic and an aqueous phase, one of the primary roles of PEG-lipids is to facilitate particle self-assembly by providing a steric barrier at the surface of nascent particles formed when nucleic acids are rapidly mixed in ethanol solutions containing lipids to bind the RNA. PEG steric hindrance prevents inter-particle fusion and promotes the formation of a homogeneous population of LNPs where diameters <100 nm can be achieved.

PEG is the most widely used and gold standard “stealth” polymer in drug delivery. PEG-lipids are typically incorporated into systems to prepare a homogenous and colloidally stable nanoparticle population due to its hydrophilic steric hindrance property (PEG shell prevents electrostatic or Van der Waals attraction that leads to aggregation). PEGylation enables to attract a water shell around the polymer shielding the RNA complex from opsonization with serum proteins, increasing serum half-life as well as reducing rapid renal clearance which results in an improvement of the pharmacokinetic behavior. Variation of the length of the acyl chains (C18, C16 or C14) of the lipids modifies the stability of the incorporation of the PEG-lipid in the particles which leads to a modulation of the pharmacokinetics. The use of a PEG-lipid containing short (C14) acyl chains that dissociates from LNPs in vivo with a halftime <30 min results in optimum hepatocyte gene-silencing potency (Chen et al., 2014, J. Control Release 196:106-12; Ambegia et al., 2005, Biochimica et Biophysica Acta 1669:155-163). In addition, tight control of particle size can be obtained by varying the PEG-lipid parameter: higher PEG MW or higher molar fraction of PEG-lipids in the particles lead to smaller particles.

Despite these advantages, PEGylation of nanoparticles may lead as well to several effects which are detrimental to the intended use for drug delivery. PEGylation of liposomes and LNPs is known to reduce the cellular uptake and endosomal escape, thus reducing at the end the overall transfection efficiency. Indeed, the PEG shell provides a steric barrier to efficient binding of particles to the cell and also hinders endosomal release by preventing membrane fusion between the liposome and the endosomal membrane. This is why the type of PEG-lipid and the amount of PEG-lipid used must be always carefully adjusted. It should provide sufficient stealth effect for in vivo and stabilization aspects on the one hand, while not hindering transfection on the other. This phenomenon is known as the “PEG Dilemma”.

Besides lowering transfection efficiency, PEGylation has been associated with accelerated blood clearance (ABC) phenomenon induced by anti-PEG antibodies and/or complement activation as well as storage diseases (Bendele A et al., 1998, Toxicolocical Sciences 42, 152-157; Young M A et al., 2007, Translational Research 149 (6), 333-342; S. M. Moghimi, J. Szebeni, 2003, Progress in Lipid Research 42:463-478). Ishida et al. and Laverman et al. reported that intravenous injection in rats of PEG-grafted liposomes may significantly alter the pharmacokinetic behavior of a second dose when this second dose is administered after an interval of several days (Laverman P et al., 2001, J. Pharmacol. Exp. Ther. 298 (2), 607-12; Ishida et al., 2006, J. Control Release 115 (3), 251-8). The phenomenon of “accelerated blood clearance” (ABC) appears to be related to the PEG content of liposomes. The presence of anti-PEG antibodies in the plasma induces a higher clearance of the particles by the Monophagocyte System (MPS) which at the end reduces the efficacy of the drug.

PEG is also supposed to induce complement activation, which can lead to hypersensitivity reaction, also known as Complement-Activation Related Pseudo-Allergy (CARPA). It is still not clear from the literature if the activation of complement is due to the nanoparticle in general or to the presence of PEG in particular.

The presence of PEG in other lipidic particles may also induce a specific immune response. Semple et al. reported that liposomes containing PEG-lipid derivatives and encapsulated antisense oligodeoxynucleotide or plasmid DNA elicit a strong immune response that results in the rapid blood clearance of subsequent doses in mice. The magnitude of this response was sufficient to induce significant morbidity and, in some instances, mortality. Rapid elimination of liposome-encapsulated oligodeoxynucleotides from blood depended on the presence of PEG-lipid in the membrane because the use of non-pegylated liposomes or liposomes containing rapidly exchangeable PEG-lipid abrogated the response. The generation of anti-PEG antibody and the putative complement activation were a likely explanation for the rapid elimination of the vesicles from the blood. (Semple et al., 2005, J. Pharmacol. Exp. Ther. 312 (3), 1020-6).

As PEG may induce immune responses there is a need to avoid it for certain applications where multiple injections are needed. Examples are therapies using mRNA, for example for protein replacement therapy. Here, the risk can be particularly high due to the potential intrinsic immunogenicity of RNA.

Thus, there remains a need in the art for efficient methods and compositions for introducing RNA into cells which avoid the disadvantages accompanied by use of PEG. The present disclosure addresses these and other needs.

The inventors surprisingly found that the RNA LNP formulations described herein fulfill the above-mentioned requirements. In particular, it is demonstrated that conjugates comprising hydrophobic chains and a polyoxazoline (POX) and/or polyoxazine (POZ) polymer are suitable components for assembly of RNA LNPs. POX and POZ can be synthesized by living cationic ring-opening polymerization using unsubstituted or substituted 2-oxazoline and 2-oxazine compounds. POX/POZ conjugates enable manufacturing of RNA LNPs with different techniques, resulting in defined surface properties and controlled size ranges. Manufacturing can be done by robust processes, compliant with the requirements for pharmaceutical manufacturing. The particles can be end-group functionalized with different moieties to modulate charge or to introduce specific molecular moieties like ligands.

### SUMMARY

In a first aspect, the present invention relates to a composition comprising lipid nanoparticles (LNPs), wherein the LNPs comprise: (i) RNA; (ii) a cationic or cationically ionizable lipid; and (iii) a conjugate of (a) a polyoxazoline (POX) and/or polyoxazine (POZ) polymer and (b) one or more hydrophobic chains.

In some embodiments, the RNA LNPs are non-viral RNA particles.

In some embodiments, the total number of POX and/or POZ repeating units in the polymer is between 2 and 200, such as between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70 POX and/or POZ repeating units. In some embodiments, the total number of POX and/or POZ repeating units in the polymer is 2 to 180, such as 4 to 160, 6 to 140, 8 to 120 or 10 to 100, e.g., 20 to 80, 30 to 70, or 40 to 50.

In some embodiments, the conjugate of (a) a POX and/or POZ polymer and (b) one or more hydrophobic chains is a conjugate of (a) a POX and/or POZ polymer and (b) 1 or 2 hydrophobic chains.

In some embodiments, the cationic or cationically ionizable lipid together with the conjugate of (a) a POX and/or POZ polymer and (b) one or more hydrophobic chains associate with RNA to form particles.

In some embodiments, the cationically ionizable lipid is positively charged only at acidic pH and does not remain cationic at physiological pH. In some embodiments, the LNPs comprise one or more additional lipids. In some embodiments, the POX and/or POZ polymer is conjugated to the one or more hydrophobic chains via a linker. In some embodiments, the one or more hydrophobic chains are one or more hydrocarbyl groups, such as non-cyclic, preferably straight hydrocarbyl groups (such as straight hydrocarbyl groups having at least 10 carbon atoms), e.g., the hydrophobic (e.g., lipophilic) chain of a natural lipid. In some embodiments, the linker comprises a functional moiety, such a cleavable moiety (e.g., a moiety which is cleavable under physiological conditions), connecting the one or more hydrophobic chains to the POX and/or POZ polymer. In some embodiments, the functional moiety is neutral at physiological conditions; in certain embodiments, the complete linker is neutral at physiological conditions.

In some embodiments, the linker comprises at least one functional moiety. In some embodiments, the linker comprises an alkylene moiety (such as a C₁₋₆ alkylene moiety, e.g., a C₁₋₃ alkylene moiety or a C₂₋₃ alkylene moiety) substituted with at least one monovalent functional moiety and/or linked, at the end by which the alkylene group is attached to the one or more hydrophobic chains, to a divalent functional moiety. In some embodiments, each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties. In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

In some embodiments, the linker does not comprise a phosphate group. In some embodiments, the linker comprises at least one moiety selected from the group consisting of ester, sulfide, disulfide, sulfone, orthoester, acylhydrazone, hydrazine, oxime, acetal, ketal, amino, and amide moieties. In some embodiments, the linker comprises at least one divalent functional moiety selected from the group consisting of ester, sulfide, sulfone, amino, and amide moieties.

In some embodiments, the linker additionally comprises an alkylene moiety (preferably a C₁₋₆ alkylene moiety) which connects the functional moiety to the POX and/or POZ polymer.

In some embodiments, the conjugate may comprise the following structure (in particular, if the one or more hydrophobic chains are attached to the N-end (i.e., the terminal N atom) of the POX and/or POZ polymer, as shown, for example in formula (II) herein below):
[(hydrophobic chain)-(divalent functional moiety)]₁₋₂-(alkylene moiety)-(POX and/or POZ polymer)).

In some embodiments, the conjugate may comprise the following structure (in particular, if the one or more hydrophobic chains are attached to the N-end (i.e., the terminal N atom) of the POX and/or POZ polymer, as shown, for example in formula (II) herein below):
(hydrophobic chain)₁₋₂-(alkylene moiety substituted with one or more monovalent functional moieties)-(POX and/or POZ polymer)

In some embodiments, the conjugate may comprise the following structure (in particular, if the one or more hydrophobic chains are attached to the N-end (i.e., the terminal N atom) of the POX and/or POZ polymer, as shown, for example in formula (II) herein below):
[(hydrophobic chain) 2-(trivalent functional moiety)]-(alkylene moiety)-(POX and/or POZ polymer).

In some embodiments (in particular those, where the one or more hydrophobic chains are attached to the N-end (i.e., the terminal N atom) of the POX and/or POZ polymer, as shown, for example in formula (II) herein below), the linker is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR²⁵)₃₋ᵣ](C₁₋₆-alkylene)-, [*—C(OR²⁵)₂O]ₚ(C₁₋₆-alkylene)-, [*—C(R²⁵)(═N—N(R²⁶)C(O)—)]ₚ(C₁₋₆-alkylene)-, [*—C(O)(N(R²⁶)—N═)C(R²⁵)—]ₚ(C₁₋₃-alkylene)-, [*═C(═N—N(R²⁶)C(O)(R²⁵))]ₚ(C₁₋₆-alkylene)-, [*—N(R²⁶)N(R²⁶)]ₚ—(C₁₋₆-alkylene)-, [*═C(═N(OH))]ₚ(C₁₋₆-alkylene)-, and [*—OC(R²⁵)(R²⁶) O]ₚ(C₁₋₆-alkylene)-, wherein * represents the attachment point of the linker to the hydrophobic chain; p is 1 or 2; C₁₋₆-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2. For example, the linker can be selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, [*—SS]ₚ(C₁₋₃-alkylene)-, [*—S(O)₂]ₚ(C₁₋₃-alkylene)-, [(*—O)ᵣ—C(OR²⁵)₃₋ᵣ](C₁₋₃-alkylene)-, [*—C(OR²⁵)₂O]ₚ(C₁₋₃-alkylene)-, [*—C(R²⁵)(═N—N(R²⁶)C(O)—)]ₚ(C₁₋₃-alkylene)-, [*—C(O)(N(R²⁶)—N═)C(R²⁵))]ₚ(C₁₋₃-alkylene)-, [*═C(═N—N(R²⁶)C(O)(R²⁵))]ₚ(C₁₋₃-alkylene)-, [*—N(R²⁶)N(R²⁶)]ₚ—(C₁₋₃-alkylene)-, [*═C(═N(OH))]ₚ(C₁₋₃-alkylene)-, and [*—OC(R²⁵)(R²⁶) O]ₚ(C₁₋₃-alkylene)-, wherein * represents the attachment point of the linker to the hydrophobic chain; p is 1 or 2; C₁₋₃-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2.

In some embodiments (in particular those, where the one or more hydrophobic chains are attached to the N-end (i.e., the terminal N atom) of the POX and/or POZ polymer, as shown, for example in formula (II) herein below), the linker can be selected from the group consisting of [*—NHC(O)]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR²⁵) 3+](C₁₋₆-alkylene)-, [*—C(OR²⁵)₂O]ₚ(C₁₋₆-alkylene)-, [*—C(R²⁵)(═N—N(R²⁶)C(O)—)]ₚ(C₁₋₆-alkylene)-, [*—C(O)(N(R²⁶)—N═)C(R²⁵)—]ₚ(C₁₋₆-alkylene)-, [*═C(═N—N(R²⁶)C(O)(R²⁵))]ₚ(C₁₋₆-alkylene)-, [*—N(R²⁶)N(R²⁶)]ₚ—(C₁₋₆-alkylene)-, [*═C(═N(OH))]ₚ(C₁₋₆-alkylene)-, and [*—OC(R²⁵)(R²⁶) O]ₚ(C₁₋₆-alkylene)-, or from the group selected of [*—NHC(O)]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR²⁵)₃₋ᵣ](C₁₋₆-alkylene)-, [*—C(OR²⁵)₂O]ₚ—(C₁₋₆-alkylene)-, [*—C(R²⁵)(═N—N(R²⁶)C(O)—)]ₚ(C₁₋₆-alkylene)-, [*—C(O)(N(R²⁶)—N═)C(R²⁵))]ₚ(C₁₋₆-alkylene)-, [*═C(═N—N(R²⁶)C(O)(R²⁵))]ₚ(C₁₋₆-alkylene)-, [*—N(R²⁶)N(R²⁶)]ₚ(C₁₋₆-alkylene)-, [*═C(═N(OH))]ₚ(C₁₋₆-alkylene)-, and [*—OC(R²⁵)(R²⁶) O]ₚ(C₁₋₆-alkylene)-, such as from the group consisting of [*—NHC(O)]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, e.g., from the group consisting of [*—NHC(O)]ₚ(C₁₋₆-alkylene)- and [*—C(O)O]ₚ(C₁₋₆-alkylene)-. In some embodiments, the linker can be selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, preferably from the group consisting of [*—NHC(O)]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-. In any of the above embodiments, it is preferred that C₁₋₆-alkylene is C₁₋₃-alkylene, such as methylene, ethylene, or trimethylene.

In some embodiments, R²⁵ is selected from the group consisting of C₁₋₃ alkyl, phenyl, and phenyl(C₁₋₃ alkyl), such as from the group consisting of methyl, ethyl, phenyl, benzyl, and phenylethyl.

In some embodiments, R²⁶ is selected from the group consisting of H, C₁₋₃ alkyl, phenyl, and phenyl(C₁₋₃ alkyl), such as from the group consisting of H, methyl, ethyl, phenyl, benzyl, and phenylethyl.

In some embodiments (in particular those, where the one or more hydrophobic chains are attached to the N-end (i.e., the terminal N atom) of the POX and/or POZ polymer, as shown, for example in formula (II) herein below), the linker can be selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), (CH₂)—CH(S—*) 2, —(CH₂)—CH(S—*)—CH₂ (S—*), *—S—(CH₂)₃—, *—S(O)₂—(CH₂)₃—, and *—OC(O)—(CH₂)—, preferably the linker is *—NHC(O)—(CH₂)— or *—NHC(O)—(CH₂)₂—, wherein * represents the attachment point of the linker to the hydrophobic chain.

In some embodiments (in particular those, where the one or more hydrophobic chains are attached to the N-end (i.e., the terminal N atom) of the POX and/or POZ polymer, as shown, for example in formula (II) herein below), the linker can be selected from the group consisting of [*—]₂NC(O)(C₁₋₆-alkylene)-, [*—C(O)O]ₚ(C₁₋₆-alkylene)-OP(O)₂O(C₁₋₆-alkylene) NHC(O)—(C₁₋₆-alkylene)-, [*—OC(O)]ₚ(C₁₋₆-alkylene)-OP(O)₂O(C₁₋₆-alkylene) NHC(O)—(C₁₋₆-alkylene)-, and [*—C(O)]ₚ(C₁₋₆-alkylene)-C(O)—, wherein * represents the attachment point of the linker to the hydrophobic chain; p is 1 or 2; and C₁₋₆-alkylene is either bivalent (if p is 1) or trivalent (if p is 2). For example, the linker can be selected from the group consisting of [*—]₂NC(O)(C₁₋₂-alkylene)-, [*—OC(O)]₂ (C₂₋₄-alkylene)-OP(O)₂O(C₂₋₃-alkylene) NHC(O)—(C₁₋₂-alkylene)-, and *—C(O)(C₂₋₃-alkylene)-C(O)—, wherein * represents the attachment point of the linker to the hydrophobic chain.

In some embodiments (in particular those, where the one or more hydrophobic chains are attached to the C-end (i.e., the terminal C atom) of the POX and/or POZ polymer, as shown, for example in formula (II′) herein below), the linker comprises at least one difunctional moiety via which the one or more hydrophobic chains are attached to the POX and/or POZ polymer. In some embodiments, the linker may additionally comprise an alkylene moiety (such as a C₁₋₆ alkylene moiety, e.g., a C₁₋₃ alkylene moiety), a cycloalkylene moiety (preferably a C₃₋₈-cycloalkylene, such as C₃₋₆-cycloalkylene moiety), or a cycloalkenylene moiety (preferably a C₃₋₈-cycloalkenylene, such as C₃₋₆-cycloalkenylene moiety) each of which connects the difunctional moiety to the POX and/or POZ polymer (either directly to the end of the POX and/or POZ polymer or, preferably, via a further difunctional moiety). For example, one hydrophobic chain may be attached to the end of the POX and/or POZ polymer via one difunctional moiety (either directly or via an alkylene, cycloalkylene, or cycloalkenylene moiety or via an alkylene, cycloalkylene, or cycloalkenylene moiety which bears another difunctional moiety); two hydrophobic chains may be attached to the end of the POX and/or POZ polymer via two difunctional moieties (which in turn are preferably attached to an alkylene, cycloalkylene, or cycloalkenylene moiety or to an alkylene, cycloalkylene, or cycloalkenylene moiety bearing another difunctional moiety); or two hydrophobic chains may be attached to the end of the POX and/or POZ polymer via the same difunctional moiety (which is then a trifunctional moiety and which may be attached to the end of the POX and/or POZ polymer either directly or via an alkylene, cycloalkylene, or cycloalkenylene moiety or to an alkylene, cycloalkylene, or cycloalkenylene moiety bearing another difunctional moiety). In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties. In some preferred embodiments (in particular those, where the one or more hydrophobic chains are attached to the C-end (i.e., the terminal C atom) of the POX and/or POZ polymer, as shown, for example in formula (II′) herein below), the linker comprises at least one divalent functional moiety selected from the group consisting of amide, sulfide, sulfone, and amino moieties.

In some embodiments, the cycloalkylene moiety is C₃₋₈-cycloalkylene, such as C₃₋₆-cycloalkylene, e.g., cyclopropylene, cyclobutylene, cyclopentylene, cyclohexylene, wherein the cycloalkylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments, the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein the cycloalkenylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments, the alkylene moiety is C₁₋₆-alkylene, such as C₁₋₃-alkylene, e.g., methylene, ethylene, or trimethylene, or C₂₋₃ alkylene.

In some embodiments (in particular those, where the one or more hydrophobic chains are attached to the C-end (i.e., the terminal C atom) of the POX and/or POZ polymer, as shown, for example in formula (II′) herein below), the conjugate comprises one of the following structures (and may have the general formula (II′)):
(hydrophobic chain)-(divalent functional moiety)-(POX and/or POZ polymer)
[(hydrophobic chain)-(divalent functional moiety)]₁₋₂-(alkylene moiety)-(divalent functional moiety)-(POX and/or POZ polymer)
(hydrophobic chain)-(divalent functional moiety)-(cycloalkylene moiety)-(divalent functional moiety)-(POX and/or POZ polymer)
(hydrophobic chain)-(divalent functional moiety)-(cycloalkenylene moiety)-(divalent functional moiety)-(POX and/or POZ polymer)
(hydrophobic chain)-(divalent functional moiety)-(alkylene moiety)-(POX and/or POZ polymer)
[(hydrophobic chain) 2-(trivalent functional moiety)]-(alkylene moiety)-(divalent functional moiety)-(POX and/or POZ polymer)

In some embodiments (in particular those, where the one or more hydrophobic chains are attached to the C-end (i.e., the terminal C atom) of the POX and/or POZ polymer, as shown, for example in formula (II′) herein below), the conjugate has one of the following formulas (and may fall within general formula (II′)):
(hydrophobic chain)-(divalent functional moiety)-(POX and/or POZ polymer)-(end group)
[(hydrophobic chain)-(divalent functional moiety)]₁₋₂-(alkylene moiety)-(divalent functional moiety)-(POX and/or POZ polymer)-(end group)
(hydrophobic chain)-(divalent functional moiety)-(cycloalkylene moiety)-(divalent functional moiety)-(POX and/or POZ polymer)-(end group)
(hydrophobic chain)-(divalent functional moiety)-(cycloalkenylene moiety)-(divalent functional moiety)-(POX and/or POZ polymer)-(end group)
(hydrophobic chain)-(divalent functional moiety)-(alkylene moiety)-(POX and/or POZ polymer)-(end group)
[(hydrophobic chain) 2-(trivalent functional moiety)]-(alkylene moiety)-(divalent functional moiety)-(POX and/or POZ polymer)-(end group)

The POX and/or POZ polymer may comprise a neutral end group (such as H, alkyl, alkoxy, ester, or amide end group) or a functionalized end group (e.g., hydroxy, thiol, cyano, azido, or amino end group). In some embodiments, the end group is R³ as defined herein (in particular with respect to any one of formulas (II), (II′), (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (VI), (VI′), (VIa), (VIa′), (VII), (VII′), (VIIa), (VIIa′), (VIII), (VIII′), (VIIIa), (IX), (IX′), and (IXa) or to any one of formulas (IIe), (IVc), (XV), (XV′), (XVI), (XVII), (XVII′), (XVIII), (XVIII′), (XIX), (XIX′), (XX′), (XXI′), (XXII′), or (XXIII′)) or R⁶ as defined herein (in particular with respect to any one of formulas (IV), (IV′), (IVa), (IVb), (IVc′), (IVd′), (IVe′), (IVf), (IVg′), (IVh′), (V), and (V′) or to formula (IVc)).

In some embodiments, in particular those, where the hydrophobic chain(s)/linker is (are) attached to the C-end, the linker can be selected from the group consisting of [*—Z]ₚ(C₁₋₆-alkylene)-Z—, *—Z—(C₃₋₈-cycloalkylene)-Z—, *—Z—(C₃₋₈-cycloalkenylene)-Z—, (*═N)(C₁₋₆-alkylene)-Z—, *—Z—(C₁₋₆-alkylene)-, and *—Z—, wherein * represents the attachment point to the hydrophobic chain(s); p is 1 or 2; C₁₋₃-alkylene is either bivalent (if p is 1) or trivalent (if p is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(C₁₋₃-alkylene) NH—, —NH(C₁₋₃-alkylene)OP(O)₂O—, —C(O)NH—, —NHC(O)—, —OC(O)NH—, —NHC(O)O—, —O—, —C(O)O—, —OC(O)—, —S—, —S(O)₂—, and —NR²²—, wherein R²² is selected from the group consisting of H, alkyl, alkenyl, alkynyl, cycloalkyl, aryl, heteroaryl, and heterocyclyl. For example, the linker can be selected from the group consisting of [*—C(O)O]ₚ(C₁₋₆-alkylene)-Z—, (*—NH) (C₁₋₆-alkylene)-Z—, (*═N)(C₁₋₆-alkylene)-Z—, (*—NH)C(O)(C₁₋₆-alkylene-Z-, (*—C(O)NH(C₁₋₆-alkylene)-Z—, (*—NH)C(O)(C₁₋₆-alkylene)-, (*—C(O)(C₁₋₆-alkylene)-Z—, *C(O)NH(C₁₋₆-alkylene)-, (*—NH)C(O)—, *—C(O)NH—, *—Z—(C₃₋₈-cycloalkenylene)-Z—, —S—, and —S(O)₂—, wherein * represents the attachment point to the hydrophobic chain(s); p is 1 or 2; C₁₋₆-alkylene is either bivalent (if p is 1) or trivalent (if p is 2); the C₃₋₈-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(C₁₋₃-alkylene) NH—, —NH(C₁₋₃-alkylene)OP(O)₂O—, —OC(O)NH—, —NHC(O)O—, —O—, —S—, and —NH—.

In some embodiments, in particular those, where the hydrophobic chain(s)/linker is (are) attached to the C-end, the linker can be selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment point to the hydrophobic chain(s); p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, the linker can be selected from the group consisting of [*—C(O)O]ₚ(C₁₋₃-alkylene)-Z—, (*—NH) (C₁₋₃-alkylene)-Z—, (*═N)(C₁₋₃-alkylene)-Z—, (*—NH)C(O)(C₁₋₃-alkylene)-Z—, (*—C(O)NH(C₁₋₃-alkylene)-Z—, (*—NH)C(O)(C₁₋₃-alkylene)-, (*—C(O)NH(C₁₋₃-alkylene)-, (*—NH)C(O)—, *—C(O)NH—, *—Z—(C₃₋₆-cycloalkenylene)-Z—, —S—, and —S(O)₂—, wherein * represents the attachment point to the hydrophobic chain(s); p is 1 or 2; C₁₋₃-alkylene is either bivalent (if p is 1) or trivalent (if p is 2); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(θ) 20 (C₁₋₂-alkylene) NH—, —NH(C₁₋₂-alkylene)OP(O)₂O—, —OC(O)NH—, —NHC(O)O—, —O—, —S—, and —NH—.

In some embodiments, in particular those, where the hydrophobic chain(s)/linker is (are) attached to the C-end, the linker can be selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment point to the hydrophobic chain(s); p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, the linker can be selected from the group consisting of [*—C(O)O]ₚ(C₁₋₃-alkylene)-Z—, (*—NH) (C₁₋₃-alkylene)-Z—, (*═N)(C₁₋₃-alkylene)-Z—, (*—NH)C(O)(C₁₋₃-alkylene)-Z—, (*—C(O)NH(C₁₋₃-alkylene)-Z—, (*—NH)C(O)(C₁₋₃-alkylene)-, (*—C(O)NH(C₁₋₃-alkylene)-, (*—NH)C(O)—, *—C(O)NH—, *—Z—(C₃₋₆-cycloalkenylene)-Z—, —S—, and —S(O)₂—, wherein * represents the attachment point to the hydrophobic chain(s); p is 1 or 2; C₁₋₃-alkylene is either bivalent (if p is 1) or trivalent (if p is 2); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH—, —NHC(O)O—, —O—, —S—, and —NH—.

In some embodiments, in particular those, where the hydrophobic chain(s)/linker is (are) attached to the C-end, the linker can be selected from the group consisting of [*—C(O)]ₚ(C₁₋₆-alkylene)-C(O)NH— and [*—]₂N—, wherein * represents the attachment point to the hydrophobic chain(s); p is 1 or 2; and C₁₋₆-alkylene is either bivalent (if p is 1) or trivalent (if p is 2). For example, the linker may be *—C(O)(C₂₋₃-alkylene)-C(O)NH— or [*—]₂N—, wherein * represents the attachment point to the hydrophobic chain(s).

In some embodiments, in particular those, where the hydrophobic chain(s)/linker is attached to the C-end, the linker can be selected from the group consisting of (*—C(O)O)(CH(OC(O)—*))(CH₂)—Z—, (*═N)(C₁₋₃-alkylene)-NHC(O)—, (*—Z)(C₁₋₃-alkylene)-Z—, *—Z—(C₃₋₆-cycloalkenylene)-Z—, and *—Z—, wherein * represents the attachment point to the hydrophobic chain(s); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, the linker can be selected from the group consisting of (*—C(O)O)(CH(OC(O)—*))(CH₂)—Z—, (*═N)(C₁₋₃-alkylene)-NHC(O)—, (*—NH) (C₁₋₃-alkylene)-NHC(O)—, *—C(O)NH—, *—NHC(O)—, *—Z—(C₃₋₆-cycloalkenylene)-Z—, —S—, and —S(O)₂—, wherein * represents the attachment point to the hydrophobic chain(s); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH—, —NHC(O)O—, —O—, —S—, and —NH—.

In some embodiments, the end group of the conjugate of (a) a POX and/or POZ polymer and (b) one or more hydrophobic chains at the side of the POX and/or POZ polymer is selected from the group consisting of H, C₁₋₆ alkyl, C₂₋₆ alkynyl, —OR²⁰, —SR²⁰ (such as SH), halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H. C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.

In some embodiments, the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor. Thus, in some embodiments, a member of a targeting pair includes one of the following: an antigen, an antibody, avidin, streptavidin, folate, transferrin, an aptamer; an RGD peptide; an NGR peptide; and galactose.

In some embodiments, the end group of the conjugate of (a) a POX and/or POZ polymer and (b) one or more hydrophobic chains at the side of the POX and/or POZ polymer is selected from the group consisting of H. C₁₋₃ alkyl, —OR²⁰, —N₃, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, —COOH, —COOCH₃, —NR²²R²³, —C(O)NR²²R²³, —NR²²C(O)R²¹, and a member of a targeting pair; R²⁰ is selected from the group consisting of H. C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, —COOH, —NH₂, —NH(C₁₋₃ alkyl), —N(C₁₋₃ alkyl)₂, and a member of a targeting pair.

In some embodiments, this end group of the conjugate at the side of the POX and/or POZ polymer is selected from the group consisting of H, C₁₋₃ alkyl, —OR²⁰, —N₃, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, —N₃, —COOH, —COOCH₃, —NH₂, —NHCH₃, —N(CH₃)₂, —C(O)NR²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group.

In some embodiments, this end group of the conjugate at the side of the POX and/or POZ polymer is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In some embodiments, the POX and/or POZ polymer comprises the following general formula (I):

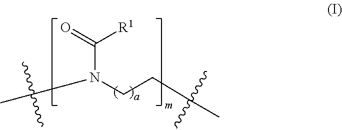

wherein a is an integer between 1 and 2; R¹ is alkyl, in particular C₁₋₃ alkyl, such as methyl, ethyl, iso-propyl, or n-propyl, and is independently selected for each repeating unit; and m is 2 to 200. In some embodiments, R¹ at each occurrence (i.e., in each repeating unit) is the same (e.g., R′ may be methyl in each repeating unit). In some embodiments, R¹ in at least one repeating unit differs from R′ in another repeating unit (e.g., for at least one repeating unit R¹ is one specific alkyl (such as ethyl), and for at least one different repeating unit R¹ is a different specific alkyl (such as methyl)).

In some embodiments, the POX and/or POZ polymer is a POX polymer and comprises repeating units of the following general formula (Ia):

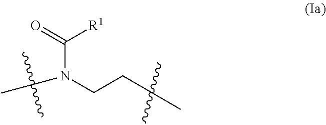

wherein R¹ is a defined above for formula (I).

In some embodiments, the POX and/or POZ polymer is a POZ polymer and comprises repeating units of the following general formula (Ib):

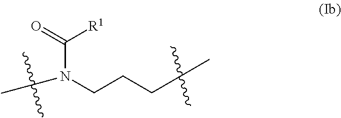

wherein R¹ is a defined above for formula (I).

In any of the above embodiments of formulas (I), (Ia), and (Ib), m (i.e., the number of repeating units of formula (Ia) or formula (Ib) in the polymer) preferably is 2 to 180, such as 4 to 160, 6 to 140, 8 to 120 or 10 to 100.

In some embodiments, the POX and/or POZ polymer is a copolymer comprising repeating units of the following general formulas (Ia) and (Ib):

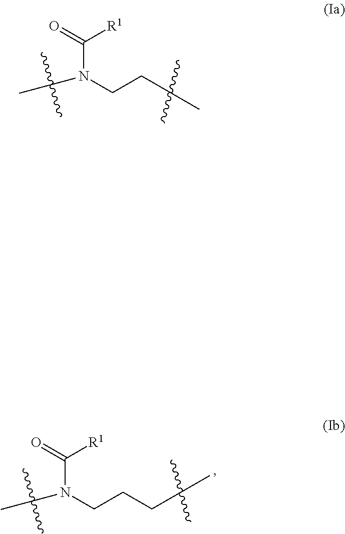

wherein R¹ is a defined above for formula (I); the number of repeating units of formula (Ia) in the copolymer is 1 to 199; the number of repeating units of formula (Ib) in the copolymer is 1 to 199; and the sum of the number of repeating units of formula (Ia) and the number of repeating units of formula (Ib) in the copolymer is 2 to 200.

Preferred embodiments of formulas (I), (Ia), and (Ib) are given herein under the heading “Conjugate of a POX and/or POZ polymer and one or more hydrophobic chains”.

In some embodiments, the conjugate has the following general formula (II) or (II′):

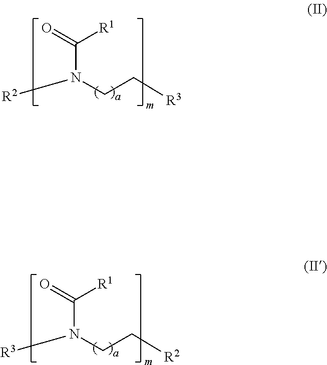

wherein:
a is an integer between 1 and 2;
R¹ is alkyl, in particular C₁₋₃ alkyl, such as methyl, ethyl, iso-propyl, or n-propyl, and is independently selected for each repeating unit;
m is 2 to 200;
R² is R⁴ or -L¹ (R⁴)ₚ, wherein each R⁴ is independently a hydrocarbyl group; L¹ is a linker; and p is 1 or 2; and
R³ is selected from the group consisting of H, C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more (such as 1, 2, 3, or 4) substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.

In some embodiments, R′ at each occurrence (i.e., in each repeating unit) is the same (e.g., R¹ may be methyl in each repeating unit). In some embodiments, R′ in at least one repeating unit differs from R¹ in another repeating unit (e.g., for at least one repeating unit R¹ is one specific alkyl (such as ethyl), and for at least one different repeating unit R¹ is a different specific alkyl (such as methyl)).

In some embodiments, a is 1, i.e., the conjugate has the following general formula (IIa) or (IIa′):

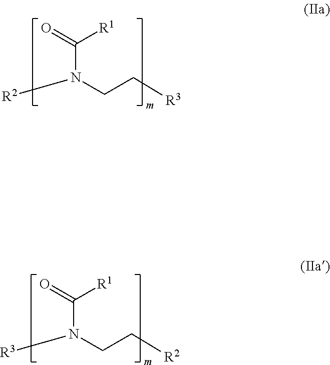

In some embodiments, a is 2, i.e., the conjugate has the following general formula (IIb) or (IIb′):

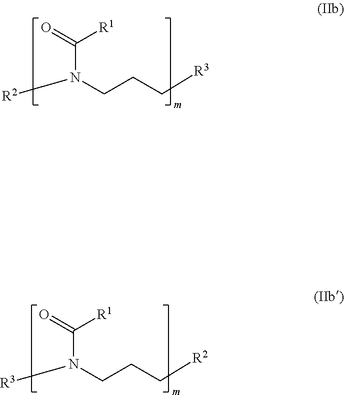

In any of the above embodiments of formulas (IIa), (IIa′), (IIb), and (IIb′), R¹, R², R³, and m are as defined for formula (II) or (II′). Preferred embodiments of formulas (II), (II′), (IIa), (IIa′), (IIb) and (IIb′) (such as formulas (IIc), (IId), (IIe′), (IIf), (IIg′), (IIh′), (IIi′), and (IIj′) as well as formula (IIe)) are given herein under the heading “Conjugate of a POX and/or POZ polymer and one or more hydrophobic chains”.

In certain embodiments, the conjugate has the following general formula (III) or (III′):

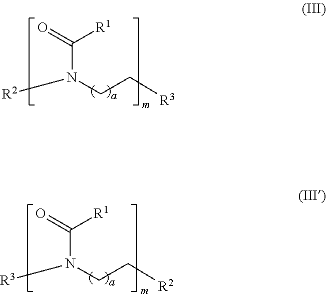

wherein:
a is an integer between 1 and 2;
R¹ is methyl or ethyl and is independently selected for each repeating unit;
m is 10 to 100 (preferably 20 to 80, 30 to 70, or 40 to 50);
R², for formula (III), is selected from the group consisting of -L′R⁴, —(CH₂)—CH(OC(O)R⁴)(CH₂OC(O)R⁴), —(CH₂)—CH(SR⁴)₂, and —(CH₂)—CH(SR⁴)—CH₂ (SR⁴), wherein each R⁴ is independently a straight hydrocarbyl group having at least 10 carbon atoms; and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L¹ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴, or R², for formula (III′), is selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In certain embodiments, the conjugate has the general formula (III) or (III′), wherein: a is an integer between 1 and 2;
R¹ is methyl or ethyl and is independently selected for each repeating unit;
m is 10 to 100;
R², for formula (III), is selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—, wherein each R⁴ is independently a straight hydrocarbyl group having at least 10 carbon atoms; or R², for formula (III′), is R⁴—C(O)(CH₂)₂C(O)NH— or R⁴N—, wherein each R⁴ is independently a straight hydrocarbyl group having at least 10 carbon atoms or a tocopherol moiety, wherein one R⁴ in the same R² moiety is a tocopherol moiety; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In some embodiments, a is 1, i.e., the conjugate has the following general formula (IIIa) or (IIIa′):

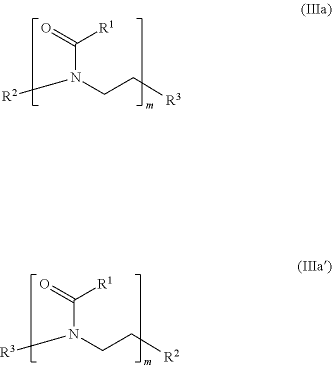

In some embodiments, a is 2, i.e., the conjugate has the following general formula (IIIb) or (IIIb′):

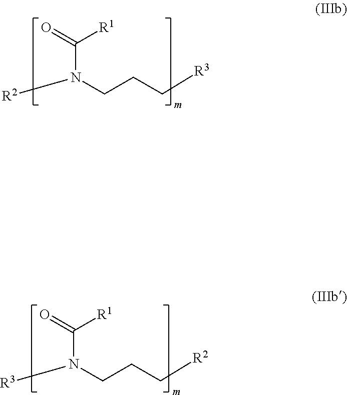

In any of the above embodiments of formulas (IIIa), (IIIa′), (IIIb), and (IIIb′), R¹, R², R³, and m are as defined for formula (III) or (III′).

Preferred embodiments of formulas (III), (III′), (IIIa), (IIIa′), (IIIb), and (IIIb′) are given herein under the heading “Conjugate of a POX and/or POZ polymer and one or more hydrophobic chains”.

In some embodiments, the conjugate has the following general formula (IV):

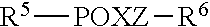

wherein:
R⁵ is R⁷ or -L² (R⁷) q, wherein each R⁷ is independently a hydrocarbyl group; L² is a linker; and q is 1 or 2;
POXZ is a copolymer containing repeating units of the following general formulas (Ia) and (Ib):

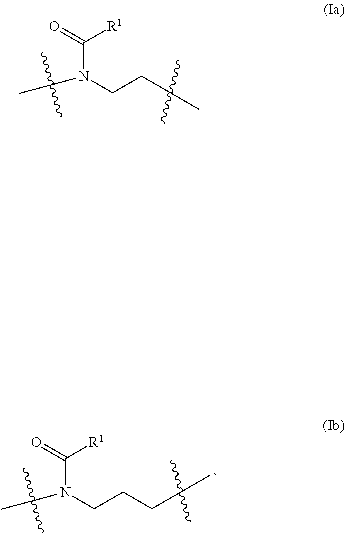

wherein each of R¹ is independently alkyl, in particular C₁₋₃ alkyl, such as methyl, ethyl, iso-propyl, or n-propyl, and is independently selected for each repeating unit; the number of repeating units of formula (Ia) in the copolymer is 1 to 199; the number of repeating units of formula (Ib) in the copolymer is 1 to 199; the sum of the number of repeating units of formula (Ia) and the number of repeating units of formula (Ib) in the copolymer is 2 to 200; and the repeating units of formulas (Ia) and (Ib) are arranged in a random, periodic, alternating or block wise manner; and
R⁶ is selected from the group consisting of H. C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.

In certain embodiments, the conjugate has the following general formula (V):

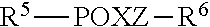

wherein:
R⁵, when attached to the N-end of the POXZ copolymer, is selected from the group consisting of -L²R⁷, —(CH₂)—CH(OC(O)R⁷)(CH₂OC(O)R⁷), —(CH₂)—CH(SR⁷) 2, and —(CH₂)—CH(SR⁷)—CH₂ (SR⁷), wherein each R⁷ is independently a straight hydrocarbyl group having at least 10 carbon atoms; and L² is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L² is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁷, or R⁵, when attached to the C-end of the POXZ copolymer, is selected from the group consisting of (R⁷C(O)O)(CH(OC(O)R⁷))(CH₂)—Z¹—, (R⁷)₂N(C₁₋₃-alkylene)-Z¹—, R⁷Z¹ (C₁₋₃-alkylene)-Z¹—, R⁷Z¹—(C₃₋₆-cycloalkenylene)-Z¹—, and R⁷Z¹, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; POXZ is a copolymer containing the repeating units of the following general formulas (Ia) and (Ib):

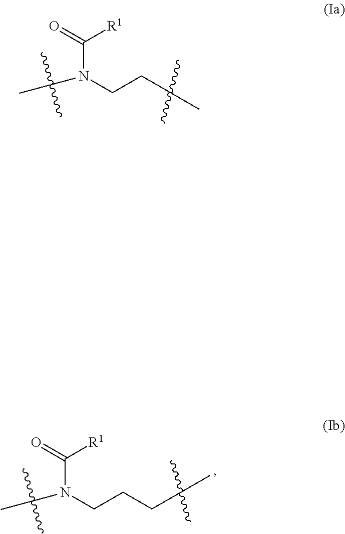

wherein each of R¹ is independently methyl or ethyl and is independently selected for each repeating unit; the number of repeating units of formula (Ia) in the copolymer is 1 to 99; the number of repeating units of formula (Ib) in the copolymer is 1 to 99; the sum of the number of repeating units of formula (Ia) and the number of repeating units of formula (Ib) in the copolymer is 10 to 100 (preferably 20 to 80, 30 to 70, or 40 to 50); and the repeating units of formulas (Ia) and (Ib) are arranged in a random, periodic, alternating or block wise manner; and
R⁶ is selected from the group consisting of H, C₁₋₃ alkyl, —OH, —N₃, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

Preferred embodiments of formulas (IV) and (V) as well as further preferred embodiments of the conjugate of a POX and/or POZ polymer and one or more hydrophobic chains (as well as formulas (IV′), (V′), (VI), (VI′), (VIa), (VIa′), (VII), (VII′), (VIIa), (VIIa′), (VIII), (VIII′), (VIIIa), (IX), (IX′), and (IXa) and formulas (IVc), (XV), (XV′), (XVI), (XVII), (XVII′), (XVIII), (XVIII′), (XIX), (XIX′), (XX′), (XXI′), (XXII′), and (XXIII′)) and particular examples thereof are given herein under the heading “Conjugate of a POX and/or POZ polymer and one or more hydrophobic chains”.

In some embodiments, the conjugate of (a) a POX and/or POZ polymer and (b) one or more hydrophobic chains inhibits aggregation of the LNPs.

In some embodiments, the conjugate of (a) a POX and/or POZ polymer and (b) one or more hydrophobic chains comprises from about 0.25 mol % to about 50 mol % of the total lipid present in the LNPs.

In some embodiments, the composition is substantially free of a lipid comprising polyethyleneglycol (PEG), preferably is substantially free of PEG.

In some embodiments, the cationically ionizable lipid comprises a head group which includes at least one nitrogen atom which is capable of being protonated under physiological conditions.

In some embodiments, the cationically ionizable lipid has the structure of Formula (X):

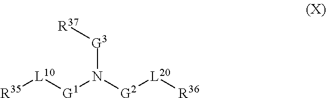

or a pharmaceutically acceptable salt, tautomer, prodrug or stereoisomer thereof, wherein: one of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆-24 alkyl or C₆₋₂₄ alkenyl;
R³⁷ is H, OR⁵⁰, CN, —C(═O)OR⁴⁰, —OC(═O) R⁴⁰ or —NR⁵⁰C(═O) R⁴⁰;
R⁴⁰ is C₁₋₁₂ alkyl;
R⁵⁰ is H or C₁₋₆ alkyl; and
x is 0, 1 or 2.

In some embodiments, the cationically ionizable lipid is selected from the structures X-1 to X-36 disclosed herein. In some embodiments, the cationically ionizable lipid is the lipid having the structure X-3.

In some embodiments, the cationically ionizable lipid is selected from the structures A to G disclosed herein.

In some embodiments, the cationically ionizable lipid has the structure of Formula (XI):

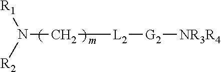

wherein
each of R₁ and R₂ is independently R₅ or -G₁-L₁-R₆, wherein at least one of R₁ and R₂ is -G₁-L₁-R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —C(═O)—, —O—, —S(O)ₓ—, —S—S—, —C(═O)S—, —SC(═O)—, —NR^{a}C(═O)—, —C(═O)NR^{a}—, —NR^{a}C(═O)NR^{a}—, —OC(═O)NR^{a}— and —NR^{a}C(═O)O—;
Ra is H or C₁₋₁₂ alkyl;
m is 0, 1, 2, 3, or 4; and
x is 0, 1 or 2.

In some embodiments, the cationically ionizable lipid has the structure (XIV-1), (XIV-2), or (XIV-3) disclosed herein.

In some embodiments, the cationic or cationically ionizable lipid comprises 2,3-dioleyloxy-1-(N,N-dimethylamino) propane (DODMA), N,N-dioleyl-N,N-dimethylammonium chloride (DODAC), N,N-distearyl-N, N-dimethylammonium bromide (DDAB), N-(1-(2,3-dioleoyloxy) propyl)-N,N,N-trimethylammonium chloride (DOTAP), N-(1-(2,3-dioleyloxy) propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), DPL14, or a mixture thereof.

In some embodiments, the cationic or cationically ionizable lipid comprises from about 20 mol % to about 80 mol % of the total lipid present in the LNPs.

In some embodiments, the LNPs further comprise one or more additional lipids, preferably selected from the group consisting of phospholipids, steroids, and combinations thereof, more preferably the LNPs comprise the cationically ionizable lipid, the conjugate, a phospholipid, and a steroid.

In some embodiments, the phospholipid is selected from the group consisting of phosphatidylcholines, phosphatidylethanolamines, phosphatidylglycerols, phosphatidic acids, phosphatidylserines and sphingomyelins, preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₋₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE). In some embodiments, the phospholipid is DOPE.

In some embodiments, the phospholipid comprises from about 1 mol % to about 30 mol % of the total lipid present in the LNPs.

In some embodiments, the steroid comprises a sterol such as cholesterol.

In some embodiments, the steroid comprises from about 10 mol % to about 60 mol % of the total lipid present in the LNPs.

In some embodiments, the cationic or cationically ionizable lipid comprises from about 20 mol % to about 70 mol % of the total lipid present in the LNPs; the conjugate of (a) a POX and/or POZ polymer and (b) one or more hydrophobic chains comprises from about 0.5 mol % to about 15 mol % of the total lipid present in the LNPs; the phospholipid comprises from about 5 mol % to about 25 mol % of the total lipid present in the LNPs; and the steroid comprises from about 25 mol % to about 55 mol % of the total lipid present in the LNPs.

In some embodiments, the LNPs have a size of from about 30 nm to about 500 nm.

In some embodiments, the RNA is encapsulated within or associated with the LNPs.

In some embodiments, the RNA is single-stranded RNA, such as mRNA.

In some embodiments, the RNA comprises a modified nucleoside in place of uridine, wherein the modified nucleoside is preferably selected from pseudouridine (ψ), N₁-methyl-pseudouridine (mlv), and 5-methyl-uridine (m5U).

In some embodiments, the RNA comprises at least one of the following, preferably all of the following: a 5′ cap: a 5′ UTR: a 3′ UTR; and a poly-A sequence.

In some embodiments, the poly-A sequence comprises at least 100 A nucleotides, wherein the poly-A sequence preferably is an interrupted sequence of A nucleotides.

In some embodiments, the 5′ cap is a cap1 or cap2 structure.

In some embodiments, the RNA encodes one or more peptides or proteins, wherein preferably the one or more peptides or proteins are therapeutic peptides or proteins and/or comprise an epitope for inducing an immune response against an antigen in a subject.

In a second aspect, the present invention relates to a method for delivering RNA to cells of a subject, the method comprising administering to a subject a composition of the first aspect.

In a third aspect, the present invention relates to a method for delivering a therapeutic peptide or protein to a subject, the method comprising administering to a subject a composition of the first aspect, wherein the RNA encodes the therapeutic peptide or protein.

In a fourth aspect, the present invention relates to a method for treating or preventing a disease or disorder in a subject, the method comprising administering to a subject a composition of the first aspect, wherein delivering the RNA to cells of the subject is beneficial in treating or preventing the disease or disorder. In a related aspect, the present invention relates to a composition of the first aspect for use in a method for treating or preventing a disease or disorder in a subject, wherein delivering the RNA to cells of the subject is beneficial in treating or preventing the disease or disorder.

In a fifth aspect, the present invention relates to a method for treating or preventing a disease or disorder in a subject, the method comprising administering to a subject a composition of the first aspect, wherein the RNA encodes a therapeutic peptide or protein and wherein delivering the therapeutic peptide or protein to the subject is beneficial in treating or preventing the disease or disorder. In a related aspect, the present invention relates to a composition of the first aspect for use in a method for treating or preventing a disease or disorder in a subject, wherein the RNA encodes a therapeutic peptide or protein and wherein delivering the therapeutic peptide or protein to the subject is beneficial in treating or preventing the disease or disorder.

In some embodiments of the second to fifth aspect, the subject is a mammal, such as a human.

In a sixth aspect, the present invention provides a conjugate of (a) a POX or POZ polymer and (b) one or more hydrophobic chains, wherein the conjugate has the general formula (II) or (II′) disclosed herein. In some embodiments, the conjugate of the sixth aspect has any one of formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48) as disclosed herein or any one of formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56).

In a seventh aspect, the present invention provides a conjugate of (a) a POX and POZ polymer and (b) one or more hydrophobic chains, wherein the POX and POZ polymer is a copolymer comprising repeating units of the following general formulas (Ia) and (Ib):

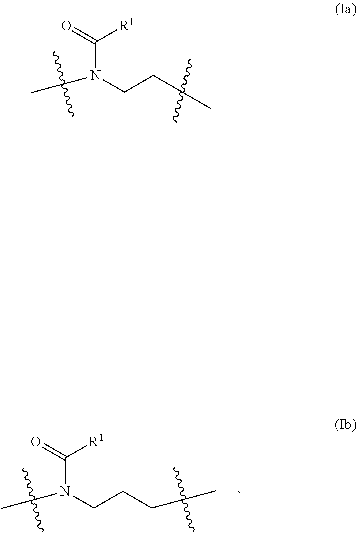

wherein each of R¹ is independently alkyl, in particular C₁₋₃ alkyl, such as methyl, ethyl, iso-propyl, or n-propyl, and is independently selected for each repeating unit; the number of repeating units of formula (Ia) in the copolymer is 1 to 199; the number of repeating units of formula (Ib) in the copolymer is 1 to 199; and the sum of the number of repeating units of formula (Ia) and the number of repeating units of formula (Ib) in the copolymer is 2 to 200. In some embodiments, the conjugate of the seventh aspect has any one of formulas (IV), (IV′), (IVa), (IVb), (IVc′), (IVd′), (IVe′), (IVf), (IVg′), (IVh′), (V), and (V′) as disclosed herein or formula (IVc) as disclosed herein.

In an eighth aspect, the present invention provides a composition comprising (i) nucleic acid (such as DNA or RNA); (ii) a cationic or cationically ionizable lipid; and (iii) a conjugate of the sixth or seventh aspect.

In some embodiments of the eighth aspect, the conjugate comprises from about 0.25 mol % to about 50 mol % of the total lipid present in the composition.

In some embodiments of the eighth aspect, the composition is substantially free of a lipid comprising polyethyleneglycol (PEG), preferably is substantially free of PEG.

In some embodiments of the eighth aspect, the cationic or cationically ionizable lipid comprises a head group which includes at least one nitrogen atom which is capable of being protonated under physiological conditions. In some embodiments of the eighth aspect, the cationic or cationically ionizable lipid has the structure of Formula (X) shown herein. In some embodiments of the eighth aspect, the cationic or cationically ionizable lipid has the structure of Formula (X) disclosed herein. In some embodiments of the eighth aspect, the cationic or cationically ionizable lipid is selected from the structures X-1 to X-36 disclosed herein. In some embodiments of the eighth aspect, the cationically ionizable lipid is the lipid having the structure X-3. In some embodiments of the eighth aspect, the cationic or cationically ionizable lipid is selected from the structures A to G disclosed herein. In some embodiments of the eighth aspect, the cationiuc or cationically ionizable lipid has the structure of Formula (XI) disclosed herein. In some embodiments of the eighth aspect, the cationic or cationically ionizable lipid has the structure (XIV-1), (XIV-2), or (XIV-3) disclosed herein. In some embodiments of the eighth aspect, the cationic or cationically ionizable lipid comprises 2,3-dioleyloxy-1-(N,N-dimethylamino) propane (DODMA), N,N-dioleyl-N,N-dimethylammonium chloride (DODAC), N,N-distearyl-N, N-dimethylammonium bromide (DDAB), N-(1-(2,3-diolcoyloxy) propyl)-N,N,N-trimethylammonium chloride (DOTAP), N-(1-(2,3-dioleyloxy) propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), DPL14, or a mixture thereof. In some embodiments of the eighth aspect, the cationic or cationically ionizable lipid comprises from about 20 mol % to about 80 mol % of the total lipid present in the composition.

In some embodiments of the eighth aspect, the composition further comprises one or more additional lipids, preferably selected from the group consisting of phospholipids, steroids, and combinations thereof, more preferably the composition comprises the cationically ionizable lipid, the conjugate, a phospholipid, and a steroid. In some embodiments of the eighth aspect, the phospholipid is selected from the group consisting of phosphatidylcholines, phosphatidylethanolamines, phosphatidylglycerols, phosphatidic acids, phosphatidylserines and sphingomyelins, preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibchenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₋₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE). In some embodiments, the phospholipid is DOPE. In some embodiments of the eighth aspect, the phospholipid comprises from about 1 mol % to about 30 mol % of the total lipid present in the composition. In some embodiments of the eighth aspect, the steroid comprises a sterol such as cholesterol. In some embodiments of the eighth aspect, the steroid comprises from about 10 mol % to about 60 mol % of the total lipid present in the composition.

In some embodiments of the eighth aspect, the cationic or cationically ionizable lipid comprises from about 20 mol % to about 70 mol % of the total lipid present in the composition; the conjugate comprises from about 0.5 mol % to about 15 mol % of the total lipid present in the composition; the phospholipid comprises from about 5 mol % to about 25 mol % of the total lipid present in the composition; and the steroid comprises from about 25 mol % to about 55 mol % of the total lipid present in the composition.

In some embodiments of the eighth aspect, the composition comprises particles dispersed in an aqueous phase, wherein the particles comprise at least a portion of the nucleic acid, at least a portion of the cationic or cationically ionizable lipid, and at least a portion of the conjugate, and, if present, at least a portion of the one or more additional lipids. In some embodiments of the eighth aspect, the particles comprise or are selected from lipid nanoparticles (LNPs), liposomes, lipoplexes (LPXs), and mixtures thereof. In some embodiments of the eighth aspect, the particles comprise or are LNPs. In some embodiments of the eighth aspect, the particles comprise or are liposomes. In some embodiments of the eighth aspect, the particles comprise or are LPXs. In some embodiments of the eighth aspect, the particles comprise or are mixtures of LNPs and liposomes. In some embodiments of the eighth aspect, the particles comprise or are mixtures of LNPs and LPXs. In some embodiments of the eighth aspect, the particles comprise or are mixtures of liposomes and LPXs. In some embodiments of the eighth aspect, the particles comprise or are mixtures of LNPs, liposomes, and LPXs. In some embodiments of the eighth aspect, the particles have a size of from about 30 nm to about 500 nm. In some embodiments of the eighth aspect, the nucleic acid is encapsulated within or associated with the particles.

In some embodiments of the eighth aspect, the nucleic acid is DNA or RNA, in particular RNA, such as single-stranded RNA, especially mRNA. In some embodiments of the eighth aspect, the RNA comprises a modified nucleoside in place of uridine, wherein the modified nucleoside is preferably selected from pseudouridine (ψ), N1-methyl-pseudouridine (m1ψ), and 5-methyl-uridine (m5U). In some embodiments of the eighth aspect, the RNA comprises at least one of the following, preferably all of the following: a 5′ cap: a 5′ UTR: a 3′ UTR; and a poly-A sequence. In some embodiments of the eighth aspect, the poly-A sequence comprises at least 100 A nucleotides, wherein the poly-A sequence preferably is an interrupted sequence of A nucleotides. In some embodiments of the eighth aspect, the 5′ cap is a cap1 or cap2 structure.

In some embodiments of the eighth aspect, the nucleic acid (such as DNA or RNA, in particular mRNA) encodes one or more peptides or proteins, wherein preferably the one or more peptides or proteins are therapeutic peptides or proteins and/or comprise an epitope for inducing an immune response against an antigen in a subject.

The composition of the eighth aspect may be used in: (i) a method for delivering nucleic acid (such as DNA or RNA) to cells of a subject (the method comprising administering to a subject a composition of the eighth aspect); (ii) a method for delivering a therapeutic peptide or protein to a subject (the method comprising administering to a subject a composition of the eighth aspect, wherein the nucleic acid (such as DNA or RNA) encodes the therapeutic peptide or protein); (iii) a method for treating or preventing a disease or disorder in a subject (the method comprising administering to a subject a composition of the eighth aspect, wherein delivering the nucleic acid (such as DNA or RNA) to cells of the subject is beneficial in treating or preventing the disease or disorder); and/or (iv) a method for treating or preventing a disease or disorder in a subject (the method comprising administering to a subject a composition of the eighth aspect, wherein the nucleic acid (such as DNA or RNA) encodes a therapeutic peptide or protein and wherein delivering the therapeutic peptide or protein to the subject is beneficial in treating or preventing the disease or disorder).

It is understood that any embodiment described herein in the context of the first, second, third, fourth, fifth, sixth, or seventh aspect may also apply to any embodiment of the eighth aspect (including the uses of any embodiment of the eighth aspect).

In a ninth aspect, the present invention provides a method of transfecting cells, comprising adding a composition of the first or eighth aspect to cells; and incubating the mixture of the composition and cells for a sufficient amount of time. In some embodiments, in particular those, where the nucleic acid (such as DNA or RNA) encodes a pharmaceutically active protein, the mixture of the composition and cells is incubated for a time sufficient to allow the expression of the pharmaceutically active protein. In some embodiments, the sufficient amount of time is at least one hour (such at least about 2 hours, at least about 3 hours, at least about 4 hours, at least about 5 hours, at least about 6 hours, at least about 9 hours, at least about 12 hours) and/or up to about 48 hours (such as up to about 36 or up to about 24 hours). In some embodiments of the ninth aspect, the method is conducted in vivo (i.e., the cells form part of an organ, a tissue and/or an organism of a subject). In some embodiments of the ninth aspect, the method is conducted in vitro (i.e., the cells do not form part of an organ, a tissue and/or an organism of a subject, e.g., the cells are an ex vivo cell culture).

It is understood that any embodiment described herein in the context of the first, second, third, fourth, fifth, sixth, seventh, or eighth aspect may also apply to any embodiment of the ninth aspect.

In a tenth aspect, the present disclosure provides a use of a composition of the first or eighth aspect for transfecting cells. In some embodiments of the tenth aspect, the use is an in vivo use (i.e., the cells form part of an organ, a tissue and/or an organism of a subject). In some embodiments of the tenth aspect, the use is an in vitro use (i.e., the cells do not form part of an organ, a tissue and/or an organism of a subject, e.g., the cells are an ex vivo cell culture).

It is understood that any embodiment described herein in the context of the first, second, third, fourth, fifth, sixth, seventh, eighth, or ninth aspect may also apply to any embodiment of the tenth aspect.

In a further aspect, the present disclosure provides a kit comprising a composition of the first or eighth aspect, a conjugate of the sixth or seventh aspect, or a pharmaceutical composition as described herein. In some embodiments, the kit is for use in therapy, such as for inducing an immune response. In some embodiments, the kit is for use in inducing an immune response against a pathogen, such as for treating or preventing an infectious disease.

### BRIEF DESCRIPTION OF THE DRAWINGS

FIG. 1: Simplified scheme for the synthesis of POX via the living cationic ring-opening polymerization using unsubstituted (R═H) or substituted (R≠H) 2-oxazoline compounds.

FIG. 2: Exemplary preparation and storage of RNA LNP compositions.

FIG. 3: Evaluation of the grafting % of poly-(2-oxazoline)-grafted-lipids into LNPs. Four different poly-(2-oxazoline)-grafted-lipids (hereinafter POX-lipids) were used to form lipid nanoparticles: C1-4-PMeOx (C14-PMeOx₄₅₋₅₀-OH; a tetradecyl alkyl chain followed by 45-50 units of poly-2-methyl-2-oxazoline); C14-PEtOx (C14-PEtOx₄₅₋₅₀-OH; a tetradecyl alkyl chain followed by 45-50 units of poly-2-ethyl-2-oxazoline); C14-NHCO—PMeOx (C14-NHCO—PMeOx₄₅₋₅₀-N₃; a tetradecyl alkyl chain linked by an amide bond to a block of 45-50 units of poly-2-methyl-2-oxazoline); and bisC14-COO—PMeOX (bisC14-COO—PMeOx₄₅₋₅₀-N₃; two tetradecyl alkyl chains, each linked via ester bonds to one single block of 45-50 units of poly-2-methyl-2-oxazoline). LNPs were prepared (using different amounts of POX-lipid) and tested with respect to the following parameters: (A) size; (B) accessible mRNA; and (C) in vitro luciferase expression.

FIG. 4: Impact of the introduction of a polar linker between the hydrophilic and hydrophobic blocks in POX-lipids. Three different stealth-grafted-lipids were used to form LNPs: the POX-lipid C14-PMeOx (C14-PMeOx₄₅₋₅₀-OH; a tetradecyl alkyl chain followed by 45-50 units of poly-2-methyl-2-oxazoline); the POX-lipid C14-NHCO—PMeOx (C14-NHCO—PMeOx₄₅₋₅₀-N₃; a tetradecyl alkyl chain linked by an amide bond to a block of 45-50 units of poly-2-methyl-2-oxazoline); and the reference stealth-lipid C14-PSar (a tetradecyl alkyl chain followed by 23 units of poly(N-methyl-glycin)). LNP formulations were analysed with respect to their size, RNA accessibility, zeta potential, and in vitro luciferase expression.

FIGS. 5: Determining the effect of the end-group in POX-lipids. Four different POX-lipids were used to form lipid nanoparticles: C14-NHCO—PMeOx-N₃ (a tetradecyl alkyl chain linked by an amide bond to a block of 45-50 units of poly-2-methyl-2-oxazoline terminated by an azido group); C14-NHCO—PMeOx-NH (a tetradecyl alkyl chain linked by an amide bond to a block of 45-50 units of poly-2-methyl-2-oxazoline terminated by a primary amino group); C14-NHCO—PMeOx-COOH (a tetradecyl alkyl chain linked by an amide bond to a block of 45-50 units of poly-2-methyl-2-oxazoline terminated by a carboxyl group); and C14-NHCO—PMeOx-COOH/NH (a physical mixture on a 1:1 ratio of C14-NHCO—PMeOx-COOH and C14-NHCO—PMeOx-NH) P formulations were analysed with respect to their size, RNA accessibility, zeta potential, and in vitro luciferase expression.

FIG. 6: Particle size and PDI of C14-PMeOX25-NH2 containing LNPs using HY501 as cationic lipid. Particle size and PDI of BM_2 LNP formulation are also included.

FIG. 7: Zeta potential of C14-PMeOX25-NH2 containing LNPs using HY501 as cationic lipid. Zeta potential of BM_2 LNP formulation is also included.

FIG. 8: Accessible RNA of C14-PMeOX25-NH2 containing LNPs using HY501 as cationic lipid measured via Ribogreen Assay.

FIG. 9: Agarose gel electrophoresis image of C14-PMeOX25-NH2 containing LNPs using HY501 as cationic lipid.

FIG. 10: Terminal complement complex (SC5b-9) formation after incubation of human serum with C14-PMeOX25-NH2 containing LNP formulations and control items. The horizontal dashed line shows the level of SC5b-9 formation for PBS.

FIG. 11: Hemolysis analysis after incubation (in neutral pH condition) of whole human blood with C14-PMeOX25-NH2 containing LNPs using HY501 as ionizable lipid.

FIG. 12: In vitro expression (FIGS. 12A-C) and viability (FIGS. 12D-F) of BM_2 and C14-PMeOX25-NH2 containing LNP in skeletal muscle cell line (C2C12) (FIGS. 12A, D), a hepatocarcinoma cell line (HepG2) (FIGS. 12B, E), and a murine macrophage cell line (Raw cell) (FIGS. 12C, F). Firefly luciferase expression 24 h post-incubation with 12.5 ng (black bars), 25 ng (grey bars) and 50 ng (white bars) per well of mRNA-loaded LNPs.

FIG. 13: Particle size and PDI of BM_2 and C14-PMeOX25-COCH₃ containing LNPs using HY501 as ionizable lipid.

FIG. 14: Zeta potential of BM_2 and C14-PMeOX25-COCH₃ containing LNPs using HY501 as ionizable lipid.

FIG. 15: Accessible RNA of BM_2 and C14-PMeOX25-COCH₃ containing LNPs using HY501 as ionizable lipid measured by Ribogreen Assay.

FIG. 16: Agarose gel electrophoresis image of C14-PMeOX25-COCH₃ containing LNPs using HY501 as ionizable lipid.

FIG. 17: Terminal complement complex (SC5b-9) formation after incubation of human serum with C14-PMeOX25-COCH₃ containing LNP formulations and control items. The horizontal dashed line shows the level of SC5b-9 formation for PBS.

FIG. 18: Hemolysis analysis after incubation (in neutral pH condition) of whole human blood with BM_2 and C14-PMeOX25-COCH₃ containing LNPs using HY501 as ionizable lipid.

FIG. 19: In vitro mRNA expression (FIGS. 19A-C) and cell viability (FIGS. 19D-F) of BM_2 and C14-PMeOX25-COCH₃ containing LNP in skeletal muscle cell line (C2C12) (FIGS. 19A, D), a hepatocarcinoma cell line (HepG2) (FIGS. 19B, E) and a murine macrophage cell line (Raw cell) (FIGS. 19C, E). Firefly luciferase mRNA expression 24 h post-incubation with 12.5 ng (black bars), 25 ng (grey bars) and 50 ng (white bars) per well of mRNA-loaded LNPs.

FIG. 20: Particle size and PDI of BM_2 and C14-PMeOX25-COOH containing LNPs using HY501 as ionizable lipid.

FIG. 21: Zeta potential of BM_2 and C14-PMeOX25-COOH containing LNPs using HY501 as ionizable lipid.

FIG. 22: Accessible RNA of C14-PMeOX25-COOH containing LNPs using HY501 as cationic lipid measured via Ribogreen Assay.

FIG. 23: Agarose gel electrophoresis image of BM_2 and C14-PMeOX25-COOH containing LNPs using HY501 as ionizable lipid.

FIG. 24: Terminal complement complex (SC5b-9) formation after incubation of human serum with C14-PMeOX25-COOH containing LNP formulations and control items. The horizontal dashed line shows the level of SC5b-9 formation for PBS.

FIG. 25: Hemolysis analysis after incubation (in neutral pH condition) of whole human blood with BM_2 and C14-PMeOX25-COOH containing LNPs using HY501 as ionizable lipid.

FIG. 26: In vitro expression (FIGS. 26A-C) and viability (FIGS. 26D-F) of BM_2 and C14-PMeOX25-COOH containing LNP in skeletal muscle cell line (C2C12) (FIGS. 26A, D), a hepatocarcinoma cell line (HepG2) (FIGS. 26B, E) and a murine macrophage cell line (Raw cell) (FIGS. 26C, F). Firefly luciferase expression 24 h post-incubation with 12.5 ng (black bars), 25 ng (grey bars) and 50 ng (white bars) per well of mRNA-loaded LNPs.

FIG. 27: Particle size and PDI of BM_2 and C14-PMeOX25-OH containing LNPs using HY501 as ionizable lipid.

FIG. 28: Zeta potential of BM_2 and C14-PMeOX25-OH containing LNPs using HY501 as ionizable lipid.

FIG. 29: Accessible RNA of BM_2 and C14-PMeOX25-OH containing LNPs.

FIG. 30: Agarose gel electrophoresis image of BM_2 and C14-PMeOX25-OH containing LNPs.

FIG. 31: Terminal complement complex (SC5b-9) formation after incubation of human serum with C14-PMeOX25-OH containing LNP formulations and control items. The horizontal dashed line shows the level of SC5b-9 formation for PBS.

FIG. 32: Hemolysis analysis after incubation (in neutral pH condition) of whole human blood with BM_2 and C14-PMeOX25-OH containing LNPs.

FIG. 33: In vitro expression (FIGS. 33A-C) and viability (FIGS. 33D-F) of BM_2 and C14-PMeOX25-OH containing LNP in skeletal muscle cell line (C2C12) (FIGS. 33A, D), a hepatocarcinoma cell line (HepG2) (FIGS. 33B, E) and a murine macrophage cell line (Raw cell) (FIGS. 33C, F). Firefly luciferase expression 24 h post-incubation with 12.5 ng (black bars), 25 ng (grey bars) and 50 ng (white bars) per well of mRNA-loaded LNPs.

FIG. 34: (A) Accessible RNA measured via Ribogreen Assay and (B) agarose gel electrophoresis image of POX-grafted lipid containing LNPs for in vivo experiment.

FIG. 35: In vitro expression (FIGS. 35A-C) and viability (FIGS. 35D-F) of BM_3 and POX-grafted lipid containing LNP in skeletal muscle cell line (C2C12) (FIGS. 35A, D), a hepatocarcinoma cell line (HepG2) (FIGS. 35B, E) and a murine macrophage cell line (Raw cell) (FIGS. 35C, F). Firefly luciferase expression 24 h post-incubation with 12.5 ng (black bars), 25 ng (grey bars) and 50 ng (white bars) per well of mRNA-loaded LNPs.

FIG. 36: In vivo whole-body bioluminescence imaging (BLI) of animals (n=3) at 6 h (upper row), 24 h (middle row) and ex-vivo BLI at 6 h (bottom row) for the tested formulations i.v.

FIG. 37: (A) In vivo luciferase expression (from in vivo imaging) 6 and 24 h post i.v. injection of the LNPs. (B) Ex vivo luciferase expression (from organ imaging) 6 h i.v. injection of the LNPs.

FIG. 38: Particle size and PDI of BM_2 and C14-PMeOX45-NH₂ containing LNPs.

FIG. 39: Zeta potential of BM_2 and C14-PMeOX45-NH₂ containing LNPs.

FIG. 40: Accessible RNA of C14-PMeOX45-NH₂ containing LNPs using HY501 as cationic lipid measured via Ribogreen Assay.

FIG. 41: Agarose gel electrophoresis image of BM_2 and C14-PMeOX45-NH₂ containing LNPs.

FIG. 42: In vitro expression (FIGS. 42A-C) and viability (FIGS. 42D-F) of BM_2 and C14-PMeOX45-NH₂ containing LNP in skeletal muscle cell line (C2C12) (FIGS. 42A, D), a hepatocarcinoma cell line (HepG2) (FIGS. 42B, E) and a murine macrophage cell line (Raw cell) (FIGS. 42C, F). Firefly luciferase expression 24 h post-incubation with 12.5 ng (black bars), 25 ng (grey bars) and 50 ng (white bars) per well of mRNA-loaded LNPs.

FIG. 43: Particle size and PDI of BM_2 and C14-PMeOX45-COOH containing LNPs.

FIG. 44: Zeta potential of BM_2 and C14-PMeOX45-COOH containing LNPs.

FIG. 45: Accessible RNA of C14-PMeOX45-COOH containing LNPs using HY501 as ionizable lipid measured by Ribogreen Assay.

FIG. 46: Agarose gel electrophoresis image of C14-PMeOX45-COOH containing LNPs using HY501 as ionizable lipid.

FIG. 47: In vitro expression (FIGS. 47A-C) and viability (FIGS. 47D-F) of BM_2 and C14-PMeOX45-COOH containing LNP in skeletal muscle cell line (C2C12) (FIGS. 47A, D), a hepatocarcinoma cell line (HepG2) (FIGS. 47B, E) and a murine macrophage cell line (Raw cell) (FIGS. 47C, F). Firefly luciferase expression 24 h post-incubation with 12.5 ng (black bars), 25 ng (grey bars) and 50 ng (white bars) per well of mRNA-loaded LNPs.

FIG. 48: Particle size and PDI of BM_2 and C14-PMeOX45-OH containing LNPs.

FIG. 49: Zeta potential of BM_2 and C14-PMeOX45-OH containing LNPs using HY501 as cationic lipid.

FIG. 50: Accessible RNA of C14-PMeOX45-OH containing LNPs using HY501 as cationic lipid measured via Ribogreen Assay.

FIG. 51: Agarose gel electrophoresis image of C14-PMeOX25-OH containing LNPs using HY501 as ionizable lipid.

FIG. 52: In vitro expression (FIGS. 52A-C) and viability (FIGS. 52D-F) of BM_2 and C14-PMeOX45-OH containing LNP in skeletal muscle cell line (C2C12) (FIGS. 52A, D), a hepatocarcinoma cell line (HepG2) (FIGS. 52B, E) and a murine macrophage cell line (Raw cell) (FIGS. 52C, F). Firefly luciferase expression 24 h post-incubation with 12.5 ng (black bars), 25 ng (grey bars) and 50 ng (white bars) per well of mRNA-loaded LNPs.

FIG. 53: Particle size and PDI of BM_2 and C14-PMeOX45-NH-AC containing LNPs.

FIG. 54: Zeta potential of BM_2 and C14-PMeOX45-NH-AC containing LNPs using HY501 as cationic lipid.

FIG. 55: Accessible RNA of C14-PMeOX45-NH-AC containing LNPs using HY501 as cationic lipid measured via Ribogreen Assay.

FIG. 56: Agarose gel electrophoresis image of C14-PMeOX45-NH-AC containing LNPs using HY501 as ionizable lipid.

FIG. 57: Particle size and PDI of BM_2 and VE-PMeOX50-CH₃ containing LNPs.

FIG. 58: Zeta potential of BM_2 and VE-PMeOX50-CH3 containing LNPs using HY501 as cationic lipid.

FIG. 59: Accessible RNA of VE-PMeOX50-CH3 containing LNPs using HY501 as cationic lipid measured via Ribogreen Assay.

FIG. 60: Agarose gel electrophoresis image of VE-PMeOX50-CH3 containing LNPs using HY501 as ionizable lipid.

### DESCRIPTION OF THE SEQUENCES

The following table provides a listing of certain sequences referenced herein

TABLE 1
DESCRIPTION OF THE SEQUENCES

| | | |
|---|---|---|
| SEQ ID NO: | Description | SEQUENCE |

Sec/MITD

| | | |
|---|---|---|
| 1 | Sec (aa) | MRVMAPRTLILLLSGALALTETWAGS |
| 2 | MITD (aa) | IVGIVAGLAVLAVVVIGAVVATVMCRRKSSGGKGGSYSQAASSDSAQGSDVSLTA |

P2P16

| | | |
|---|---|---|
| 3 | P2P16 (aa) | KKQYIKANSKFIGITELKKLGGGKRGGGKKMTNSVDDALINSTKIYSYFPSVISKVNQGAQ |

GS Linker

| | | |
|---|---|---|
| 4 | GS Linker 1 | GGSGGGGSGG |
| 5 | GS Linker 2 | GSSGGGGSPGGGSS |

5′-UTR (hAg-Kozak)

| | | |
|---|---|---|
| 6 | 5′-UTR | AACUAGUAUUCUUCUGGUCCCCACAGACUCAGAGAGAACCCGCCACC |

3′-UTR (FI element)

| | | |
|---|---|---|
| 7 | 3′-UTR | CUGGUACUGCAUGCACGCAAUGCUAGCUGCCCCUUUCCCGUCCUGGGUACCCCGAGUCUCC |
| | | CACCUCCACCUGCCCCACUCACCACCUCUGCUAGUUCCAGACACCUCCCAAGCACGCAGCA |
| | | ACACCCCCACGGGAAACAGCAGUGAUUAACCUUUAGCAAUAAACGAAAGUUUAACUAAGCU |
| | | GUGCCAGCCACACC |

A30L70

| | | |
|---|---|---|
| 8 | A30L70 | AAAAAAAAAAAAAAAAAAAAAAAAAAAAAAGCAUAUGACUAAAAAAAAAAAAAAAAAAAAA |
| | | AAAAAAAAAAAAAAAAAAAAAA |

Helper epitopes

| | | |
|---|---|---|
| 9 | P2 | QYIKANSKFIGITEL |
| 10 | P16 | MTNSVDDALINSTKIYSYFPSVISKVNQGAQG |

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described in more detail. These elements are listed with specific embodiments, however, it should be understood that they may be combined in any manner and in any number to create additional embodiments. The variously described examples and preferred embodiments should not be construed to limit the present disclosure to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. For example, if in one embodiment the composition comprises a conjugate of formula (III) and in another embodiment the cationically ionizable lipid has one of the structures XIV-1, XIV-2, and XIV-3, then in a further embodiment the composition comprises a conjugate of formula (III) and the cationically ionizable lipid having one of the structures XIV-1, XIV-2, and XIV-3.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Organikum, Deutscher Verlag der Wissenschaften, Berlin 1990; Streitwieser/Heathcook, “Organische Chemie”, VCH, 1990; Beyer/Walter, “Lehrbuch der Organischen Chemie”, S. Hirzel Verlag Stuttgart, 1988; Carey/Sundberg, “Organische Chemie”, VCH, 1995; March, “Advanced Organic Chemistry”, John Wiley & Sons, 1985; Rompp Chemie Lexikon, Falbe/Regitz (Hrsg.), Georg Thieme Verlag Stuttgart, New York, 1989; Molecular Cloning: A Laboratory Manual, 2nd Edition, J. Sambrook et al. eds., Cold Spring Harbor Laboratory Press, Cold Spring Harbor 1989).

All methods described herein can be performed in any suitable order unless otherwise indicated herein or otherwise clearly contradicted by the context. The use of any and all examples, or exemplary language (e.g., “such as”), provided herein is intended merely to better illustrate the present disclosure and does not pose a limitation on the scope of the present disclosure otherwise claimed. No language in the specification should be construed as indicating any non-claimed element essential to the practice of the present disclosure.

Recitation of ranges of values herein is merely intended to serve as a shorthand method of referring individually to each separate value falling within the range. Unless otherwise indicated herein, each individual value is incorporated into the specification as if it were individually recited herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

### Definitions

In the following, definitions will be provided which apply to all aspects of the present disclosure. The following terms have the following meanings unless otherwise indicated. Any undefined terms have their art recognized meanings.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The term “consisting essentially of” means excluding other members, integers or steps of any essential significance. The term “comprising” encompasses the term “consisting essentially of” which, in turn, encompasses the term “consisting of”. Thus, at each occurrence in the present application, the term “comprising” may be replaced with the term “consisting essentially of” or “consisting of”. Likewise, at each occurrence in the present application, the term “consisting essentially of” may be replaced with the term “consisting of”.

The terms “a” and “an” and “the” and similar reference used in the context of describing the disclosure (especially in the context of the claims) are to be construed to cover both the singular and the plural, unless otherwise indicated herein or clearly contradicted by context.

Where used herein, “and/or” is to be taken as specific disclosure of each of the two specified features or components with or without the other. For example, “X and/or Y” is to be taken as specific disclosure of each of (i) X, (ii) Y, and (iii) X and Y, just as if each is set out individually herein.

In the context of the present disclosure, the term “about” denotes an interval of accuracy that the person of ordinary skill will understand to still ensure the technical effect of the feature in question. The term typically indicates deviation from the indicated numerical value by ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, and for example ±0.01%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect.

Terms such as “reduce” or “inhibit” as used herein means the ability to cause an overall decrease, for example, of about 5% or greater, about 10% or greater, about 15% or greater, about 20% or greater, about 25% or greater, about 30% or greater, about 40% or greater, about 50% or greater, or about 75% or greater, in the level. The term “inhibit” or similar phrases includes a complete or essentially complete inhibition, i.e. a reduction to zero or essentially to zero.

Terms such as “increase” or “enhance” in some embodiments relate to an increase or enhancement by at least about 10%, at least about 20%, at least about 30%, at least about 40%, at least about 50%, at least about 80%, or at least about 100%.

“Physiological pH” as used herein refers to a pH of about 7.4. In some embodiments, “physiological pH” as used herein refers to a neutral pH, i.e., a pH of about 7.0.

“Physiological conditions” as used herein refer to the conditions (in particular pH and temperature) in a living subject, in particular a human. Preferably, physiological conditions mean a physiological pH and/or a temperature of about 37° C.

As used in the present disclosure, “% w/v” refers to weight by volume percent, which is a unit of concentration measuring the amount of solute in grams (g) expressed as a percent of the total volume of solution in milliliters (mL).

As used in the present disclosure, “% by weight” or “% (w/w)” (or “% w/w”) refers to weight percent, which is a unit of concentration measuring the amount of a substance in grams (g) expressed as a percent of the total weight of the total composition in grams (g).

As used in the present disclosure, “mol %” is defined as the ratio of the number of moles of one component to the total number of moles of all components, multiplied by 100.

The term “ionic strength” refers to the mathematical relationship between the number of different kinds of ionic species in a particular solution and their respective charges. Thus, ionic strength I is represented mathematically by the formula

$$I = \frac{1}{2} \cdot {\sum }_{i} {z}_{i}^{2} \cdot {c}_{i}$$

in which c is the molar concentration of a particular ionic species and z the absolute value of its charge. The sum Σ is taken over all the different kinds of ions (i) in solution.

According to the disclosure, the term “ionic strength” in some embodiments relates to the presence of monovalent ions. Regarding the presence of divalent ions, in particular divalent cations, their concentration or effective concentration (presence of free ions) due to the presence of chelating agents is in some embodiments sufficiently low so as to prevent degradation of the RNA. In some embodiments, the concentration or effective concentration of divalent ions is below the catalytic level for hydrolysis of the phosphodiester bonds between RNA nucleotides. In some embodiments, the concentration of free divalent ions is 20 UM or less. In some embodiments, there are no or essentially no free divalent ions.

A “monovalent” compound relates to a compound having only one functional group of interest. For example, a monovalent anion relates to a compound having only one negatively charged group, preferably under physiological conditions.

A “divalent” or “dibasic” compound relates to a compound having two functional groups of interest. For example, a dibasic organic acid has two acid groups. An example of a divalent cation is Ca²⁺.

A “polyvalent” or “polybasic” compound relates to a compound having three or more functional groups of interest. For example, a polybasic organic acid has three or more acid groups.

A “monovalent moiety” relates to a monoradical, i.e., a moiety having a valence of 1. Typical monovalent moieties include alkyl, alkenyl, aryl, etc.

A “divalent moiety” or “bivalent moiety” relates to a diradical, i.e., a moiety having a valence of 2. Typical divalent moieties include alkylene, alkenylene, cycloalkylene, cycloalkenylene, arylene, etc. A further example of a divalent moiety is the C₁₋₆-alkylene moiety in the group [*—S]ₚ(C₁₋₆-alkylene)-, if p is 1 (resulting in the group *—S(C₁₋₆-alkylene)-, such as *—S—(CH₂)₆— or *—S—CH₂—, wherein * represents the attachment point to R⁴ or R⁷).

A “polyvalent moiety” relates to a polyradical, i.e., a moiety having a valence of at least 3. E.g., a “trivalent moiety” relates to a triradical, i.e., a moiety having a valence of 3. For example, by removing a further H atom of an alkylene group the resulting alkylene is trivalent. A further example of a trivalent moiety is the C₁₋₆-alkylene moiety in the group [*—S]ₚ(C₁₋₆-alkylene)-, if p is 2 (resulting in the group [*—S]₂ (C₁₋₆-alkylene)-, such as *—S—CH(S—*) (CH₂)₅— or *—S—CH(S—*) (CH₂)—, wherein * represents the attachment point to R⁴ or R⁷). Another example of a trivalent moiety is the N atom in the group (*═N)(C₁₋₃-alkylene)-Z— (here two hydrophobic chains are bound to the N atom which, in turn, is attached to the C₁₋₃-alkylene moiety).

“Osmolality” refers to the concentration of solutes expressed as the number of osmoles of solute per kilogram of solvent.

The term “freezing” relates to the phase transition from the liquid to the solid state. It usually occurs on lowering the temperature of a system below a critical temperature and is accompanied by a characteristic change of enthalpy of the system.

The term “lyophilizing” or “lyophilization” refers to the freeze-drying of a substance by freezing it and then reducing the surrounding pressure to allow the frozen medium in the substance to sublimate directly from the solid phase to the gas phase.

The term “spray-drying” refers to spray-drying a substance by mixing (heated) gas with a fluid that is atomized (sprayed) within a vessel (spray dryer), where the solvent from the formed droplets evaporates, leading to a dry powder.

The term “cryoprotectant” relates to a substance that is added to a formulation in order to protect the active ingredients during the freezing stages.

The term “lyoprotectant” relates to a substance that is added to a formulation in order to protect the active ingredients during the drying stages.

The term “reconstitute” relates to adding a solvent such as water to a dried product to return it to a liquid state such as its original liquid state.

The term “recombinant” in the context of the present disclosure means “made through genetic engineering”. In some embodiments, a “recombinant object” in the context of the present disclosure is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring. The term “found in nature” means “present in nature” and includes known objects as well as objects that have not yet been discovered and/or isolated from nature, but that may be discovered and/or isolated in the future from a natural source.

As used herein, the terms “room temperature” and “ambient temperature” are used interchangeably herein and refer to temperatures from at least about 15° C. preferably from about 15° C. to about 35° C. from about 15° C. to about 30° C., from about 15° C. to about 25° C., or from about 17° C. to about 22° C. Such temperatures will include 15° C., 16° C., 17° C. 18° C., 19° C., 20° C., 21° C. and 22° C.

The term “alkyl” refers to a monoradical of a saturated straight or branched hydrocarbon. Preferably, the alkyl group comprises from 1 to 14 (such as 1 to 12 or 1 to 10) carbon atoms, i.e., 1, 2, 3, 4, 5, 6, 7, 8, 9, 10, 11, 12, 13, or 14 carbon atoms, abbreviated as C₁₋₁₄ alkyl, (such as 1, 2, 3, 4, 5, 6, 7, 8, 9, or 10 carbon atoms, abbreviated as C₁₋₁₀ alkyl), more preferably 1 to 8 carbon atoms, such as 1 to 6 or 1 to 4 carbon atoms. Exemplary alkyl groups include methyl, ethyl, propyl, iso-propyl (also called 2-propyl or 1-methylethyl), butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, n-heptyl, iso-heptyl, n-octyl, 2-ethyl-hexyl, n-nonyl, n-decyl, n-undecyl, n-dodecyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1st level substituent, a 2nd level substituent, or a 3rd level substituent as specified herein. Examples of a substituted alkyl include chloromethyl, dichloromethyl, fluoromethyl, and difluoromethyl.

The term “alkylene” refers to a diradical of a saturated straight or branched hydrocarbon. Preferably, the alkylene comprises from 1 to 12 (such as 1 to 10) carbon atoms, i.e., 1, 2, 3, 4, 5, 6, 7, 8, 9, 10, 11, or 12 carbon atoms (such as 1, 2, 3, 4, 5, 6, 7, 8, 9, or 10 carbon atoms), more preferably 1 to 8 carbon atoms, such as 1 to 6 or 1 to 4 carbon atoms. Exemplary alkylene groups include methylene, ethylene (i.e., 1,1-ethylene, 1,2-ethylene), propylene (i.e., 1,1-propylene, 1,2-propylene (—CH(CH₃)CH₂—), 2,2-propylene (—C(CH₃)₂—), and 1,3-propylene), the butylene isomers (e.g., 1,1-butylene, 1,2-butylene, 2,2-butylene, 1,3-butylene, 2,3-butylene (cis or trans or a mixture thereof), 1,4-butylene, 1,1-iso-butylene, 1,2-iso-butylene, and 1,3-iso-butylene), the pentylene isomers (e.g., 1,1-pentylene, 1,2-pentylene, 1,3-pentylene, 1,4-pentylene, 1,5-pentylene, 1,1-iso-pentylene, 1,1-sec-pentyl, 1,1-neo-pentyl), the hexylene isomers (e.g., 1,1-hexylene, 1,2-hexylene, 1,3-hexylene, 1,4-hexylene, 1,5-hexylene, 1,6-hexylene, and 1,1-isohexylene), the heptylene isomers (e.g., 1,1-heptylene, 1,2-heptylene, 1,3-heptylene, 1,4-heptylene, 1,5-heptylene, 1,6-heptylene, 1,7-heptylene, and 1,1-isoheptylene), the octylene isomers (e.g., 1,1-octylene, 1,2-octylene, 1,3-octylene, 1,4-octylene, 1,5-octylene, 1,6-octylene, 1,7-octylene, 1,8-octylene, and 1,1-isooctylene), and the like. The straight alkylene moieties having at least 3 carbon atoms and a free valence at each end can also be designated as a multiple of methylene (e.g., 1,4-butylene can also be called tetramethylene). Generally, instead of using the ending “ylene” for alkylene moieties as specified above, one can also use the ending “diyl” (e.g., 1,2-butylene can also be called butan-1,2-diyl). A “substituted alkylene” means that one or more (such as 1 to the maximum number of hydrogen atoms bound to an alkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1st level substituent, a 2nd level substituent, or a 3rd level substituent as specified herein.

The term “alkenyl” refers to a monoradical of an unsaturated straight or branched hydrocarbon having at least one carbon-carbon double bond. Generally, the maximal number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 to 6 (such as 1 to 4), i.e., 1, 2, 3, 4, 5, or 6, carbon-carbon double bonds. Preferably, the alkenyl group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in certain embodiments, the alkenyl group comprises from 2 to 12, abbreviated as C₂₋₁₂ alkenyl, (e.g., 2 to 10) carbon atoms and 1, 2, 3, 4, 5, or 6 (e.g., 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenyl groups include vinyl, 1-propenyl, 2-propenyl (i.e., allyl), 1-butenyl, 2-butenyl, 3-butenyl, 1-pentenyl, 2-pentenyl, 3-pentenyl, 4-pentenyl. 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl. 2-decenyl, 3-decenyl, 4-decenyl, 5-decenyl, 6-decenyl, 7-decenyl, 8-decenyl, 9-decenyl, 1-undecenyl. 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl. 6-dodecenyl, 7-dodecenyl, 8-dodecenyl, 9-dodecenyl, 10-dodecenyl, 11-dodecenyl, and the like. If an alkenyl group is attached to a nitrogen atom, the double bond cannot be alpha to the nitrogen atom. A “substituted alkenyl” means that one or more (such as 1 to the maximum number of hydrogen atoms bound to an alkenyl group, e.g., 1, 2, 3, 4, 5, 6, 7, 8, 9, or up to 10, such as between 1 to 5, 1 to 4, or 1 to 3, or 1 or 2) hydrogen atoms of the alkenyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The term “alkenylene” refers to a diradical of an unsaturated straight or branched hydrocarbon having at least one carbon-carbon double bond. Generally, the maximal number of carbon-carbon double bonds in the alkenylene group can be equal to the integer which is calculated by dividing the number of carbon atoms in the alkenylene group by 2 and, if the number of carbon atoms in the alkenylene group is uneven, rounding the result of the division down to the next integer. For example, for an alkenylene group having 9 carbon atoms, the maximum number of carbon-carbon double bonds is 4. Preferably, the alkenylene group has 1 to 6 (such as 1 to 4), i.e., 1, 2, 3, 4, 5, or 6, carbon-carbon double bonds. Preferably, the alkenylene group comprises from 2 to 12 (such as 2 to 10) carbon atoms, i.e., 2, 3, 4, 5, 6, 7, 8, 9, 10, 11, or 12 carbon atoms (such as 2, 3, 4, 5, 6, 7, 8, 9, or 10 carbon atoms), more preferably 2 to 8 carbon atoms, such as 2 to 6 carbon atoms or 2 to 4 carbon atoms. Thus, in certain embodiments, the alkenylene group comprises from 2 to 12 (such as 2 to 10 carbon) atoms and 1, 2, 3, 4, 5, or 6 (such as 1, 2, 3, 4, or 5) carbon-carbon double bonds, more preferably it comprises 2 to 8 carbon atoms and 1, 2, 3, or 4 carbon-carbon double bonds, such as 2 to 6 carbon atoms and 1, 2, or 3 carbon-carbon double bonds or 2 to 4 carbon atoms and 1 or 2 carbon-carbon double bonds. The carbon-carbon double bond(s) may be in cis (Z) or trans (E) configuration. Exemplary alkenylene groups include ethen-1,2-diyl, vinylidene (also called ethenylidene), 1-propen-1,2-diyl, 1-propen-1,3-diyl, 1-propen-2,3-diyl, allylidene, 1-buten-1,2-diyl, 1-buten-1,3-diyl, 1-buten-1,4-diyl, 1-buten-2,3-diyl, 1-buten-2,4-diyl, 1-buten-3,4-diyl, 2-buten-1,2-diyl, 2-buten-1,3-diyl, 2-buten-1,4-diyl. 2-buten-2,3-diyl, 2-buten-2,4-diyl, 2-buten-3,4-diyl, and the like. If an alkenylene group is attached to a nitrogen atom, the double bond cannot be alpha to the nitrogen atom. A “substituted alkenylene” means that one or more (such as 1 to the maximum number of hydrogen atoms bound to an alkenylene group, e.g., 1, 2, 3, 4, 5, 6, 7, 8, 9, or up to 10, such as between 1 to 5, 1 to 4, or 1 to 3, or 1 or 2) hydrogen atoms of the alkenylene group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The term “alkynyl” refers to a monoradical of an unsaturated straight or branched hydrocarbon having at least one carbon-carbon triple bond. Generally, the maximal number of carbon-carbon triple bonds in the alkynyl group can be equal to the integer which is calculated by dividing the number of carbon atoms in the alkynyl group by 2 and, if the number of carbon atoms in the alkynyl group is uneven, rounding the result of the division down to the next integer. For example, for an alkynyl group having 9 carbon atoms, the maximum number of carbon-carbon triple bonds is 4. Preferably, the alkynyl group has 1 to 6 (such as 1 to 4), i.e., 1, 2, 3, 4, 5, or 6, more preferably 1 or 2 carbon-carbon triple bonds. Preferably, the alkynyl group comprises from 2 to 12 (such as 2 to 10) carbon atoms (such as 1, 2, 3, 4, 5, 6, 7, 8, 9, or 10 carbon atoms), i.e., 2, 3, 4, 5, 6, 7, 8, 9, 10, 11, or 12 carbon atoms, more preferably 2 to 8 carbon atoms, such as 2 to 6 carbon atoms or 2 to 4 carbon atoms. Thus, in certain embodiments, the alkynyl group comprises from 2 to 12 (such as 2 to 10) carbon atoms and 1, 2, 3, 4, 5, or 6 (such as 1, 2, 3, 4, or 5 (preferably 1, 2, or 3)) carbon-carbon triple bonds, more preferably it comprises 2 to 8 carbon atoms and 1, 2, 3, or 4 (preferably 1 or 2) carbon-carbon triple bonds, such as 2 to 6 carbon atoms and 1, 2 or 3 carbon-carbon triple bonds or 2 to 4 carbon atoms and 1 or 2 carbon-carbon triple bonds. Exemplary alkynyl groups include ethynyl, 1-propynyl, 2-propynyl, 1-butynyl, 2-butynyl, 3-butynyl, 1-pentynyl, 2-pentynyl. 3-pentynyl. 4-pentynyl, 1-hexynyl, 2-hexynyl, 3-hexynyl. 4-hexynyl, 5-hexynyl, 1-heptynyl, 2-heptynyl, 3-heptynyl, 4-heptynyl, 5-heptynyl, 6-heptynyl, 1-octynyl, 2-octynyl, 3-octynyl, 4-octynyl, 5-octynyl, 6-octynyl, 7-octynyl, 1-nonylyl, 2-nonynyl, 3-nonynyl, 4-nonynyl, 5-nonynyl, 6-nonynyl, 7-nonynyl, 8-nonynyl, 1-decynyl, 2-decynyl, 3-decynyl, 4-decynyl, 5-decynyl, 6-decynyl, 7-decynyl, 8-decynyl, 9-decynyl, and the like. If an alkynyl group is attached to a nitrogen atom, the triple bond cannot be alpha to the nitrogen atom. A “substituted alkynyl” means that one or more (such as 1 to the maximum number of hydrogen atoms bound to an alkynyl group, e.g., 1, 2, 3, 4, 5, 6, 7, 8, 9, or up to 10, such as between 1 to 5, 1 to 4, or 1 to 3, or 1 or 2) hydrogen atoms of the alkynyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The term “cycloalkyl” or “cycloaliphatic” represents cyclic non-aromatic versions of “alkyl” and “alkenyl” with preferably 3 to 14 carbon atoms, such as 3 to 10 carbon atoms, i.e., 3, 4, 5, 6, 7, 8, 9, or 10 carbon atoms, more preferably 3 to 7 carbon atoms. In some embodiments, the cycloalkyl group has 1, 2, or more (preferably 1 or 2) double bonds. Exemplary cycloalkyl groups include cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, cyclononyl, cyclononenyl, cylcodecyl, cylcodecenyl, and adamantyl. The term “cycloalkyl” is also meant to include bicyclic and tricyclic versions thereof. If bicyclic rings are formed it is preferred that the respective rings are connected to each other at two adjacent carbon atoms, however, alternatively the two rings are connected via the same carbon atom, i.e., they form a spiro ring system or they form “bridged” ring systems. Preferred examples of cycloalkyl include C₃-C₈-cycloalkyl, in particular cyclopropyl, cyclobutyl, cyclopentyl, cyclopentenyl, cyclohexyl, cyclohexenyl, cyclohexadienyl, cycloheptyl, cyclooctyl, spiro[3,3]heptyl, spiro[3,4]octyl, spiro[4,3]octyl, spiro[4,5]decanyl, bicyclo[4.1.0]heptyl, bicyclo[3.2.0]heptyl, bicyclo[2.2.1]heptyl (i.e., norbornyl), bicyclo[2.2.2]octyl, bicyclo[5.1.0]octyl, bicyclo[4.2.0]octyl, bicyclo[4.3.0]nonyl. 1,2,3,4-tetrahydronaphthyl (i.e., tetralinyl), and bicyclo[4.4.0]decanyl (i.e., decalinyl). A “substituted cycloalkyl” means that one or more (such as 1 to the maximum number of hydrogen atoms bound to a cycloalkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The term “cycloalkylene” represents cyclic non-aromatic versions of “alkylene” and is a geminal, vicinal or isolated diradical. In certain embodiments, the cycloalkylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In some embodiments, the cycloalkylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered) cycloalkylene. Generally, instead of using the ending “ylene” for cycloalkylene moieties as specified above, one can also use the ending “diyl” (e.g., 1,2-cyclopropylene can also be called cyclopropan-1,2-diyl). Exemplary cycloalkylene groups include cyclohexylene, cycloheptylene, cyclopropylene, cyclobutylene, cyclopentylene, cyclooctylene, bicyclo[3.2.1]octylene, bicyclo[3.2.2]nonylene, and adamantanylene (e.g., tricyclo[3.3.1.13.7]decan-2,2-diyl). A “substituted cycloalkylene” means that one or more (such as 1 to the maximum number of hydrogen atoms bound to an cycloalkylene group, e.g., 1, 2, 3, 4, 5, 6, 7, 8, 9, or up to 10, such as between 1 to 5, 1 to 4, or 1 to 3, or 1 or 2) hydrogen atoms of the alkylene group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The term “cycloalkenylene” represents cyclic non-aromatic versions of “alkenylene” and is a geminal, vicinal or isolated diradical. Generally, the maximal number of carbon-carbon double bonds in the cycloalkenylene group can be equal to the integer which is calculated by dividing the number of carbon atoms in the cycloalkenylene group by 2 and, if the number of carbon atoms in the cycloalkenylene group is uneven, rounding the result of the division down to the next integer. For example, for an cycloalkenylene group having 9 carbon atoms, the maximum number of carbon-carbon double bonds is 4. Preferably, the cycloalkenylene group has 1 to 6 (such as 1 to 4), i.e., 1, 2, 3, 4, 5, or 6, carbon-carbon double bonds. In certain embodiments, the cycloalkenylene (i) is monocyclic or polycyclic (such as bi- or tricyclic) and/or (ii) is 3- to 14-membered (i.e., 3-, 4-, 5-, 6-, 7-, 8-, 9-, 10-, 11-, 12-, 13-, or 14-membered, such as 3- to 12-membered or 3- to 10-membered). In some embodiments, the cycloalkenylene is a mono-, bi- or tricyclic 3- to 14-membered (i.e., 3-, 4-, 5-, 6-, 7-, 8-, 9-, 10-, 11-, 12-, 13-, or 14-membered, such as 3- to 12-membered or 3- to 10-membered) cycloalkenylene. Exemplary cycloalkenylene groups include cyclohexenylene, cycloheptenylene, cyclopropenylene, cyclobutenylene, cyclopentenylene, and cyclooctenylene. A “substituted cycloalkenylene” means that one or more (such as 1 to the maximum number of hydrogen atoms bound to an cycloalkenylene group, e.g., 1, 2, 3, 4, 5, 6, 7, 8, 9, or up to 10, such as between 1 to 5, 1 to 4, or 1 to 3, or 1 or 2) hydrogen atoms of the cycloalkenylene group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The term “aryl” or “aromatic ring” refers to a monoradical of an aromatic cyclic hydrocarbon. Preferably, the aryl group contains 3 to 14 (e.g., 5, 6, 7, 8, 9, or 10, such as 5, 6, or 10) carbon atoms which can be arranged in one ring (e.g., phenyl) or two or more condensed rings (e.g., naphthyl). Exemplary aryl groups include cyclopropenylium, cyclopentadienyl, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. Aryl does not encompass fullerenes. A “substituted aryl” means that one or more (such as 1 to the maximum number of hydrogen atoms bound to an aryl group, e.g., 1, 2, 3, 4, 5, 6, 7, 8, 9, or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein. Examples of a substituted aryl include biphenyl, 2-fluorophenyl, 2-chloro-6-methylphenyl, anilinyl, 4-hydroxyphenyl, and methoxyphenyl (i.e., 2-, 3-, or 4-methoxyphenyl).

The term “heteroaryl” or “heteroaromatic ring” means an aryl group as defined above in which one or more carbon atoms in the aryl group are replaced by heteroatoms of O. S. or N. Preferably, heteroaryl refers to a five or six-membered aromatic monocyclic ring wherein 1, 2, or 3 carbon atoms are replaced by the same or different heteroatoms of O, N, or S. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-memered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The term “heterocyclyl” or “heterocyclic ring” means a cycloalkyl group as defined above in which from 1, 2, 3, or 4 carbon atoms in the cycloalkyl group are replaced by heteroatoms of oxygen, nitrogen, silicon, selenium, phosphorous, or sulfur, preferably O, S, or N. A heterocyclyl group has preferably 1 or 2 rings containing from 3 to 10, such as 3, 4, 5, 6, or 7, ring atoms. Preferably, in each ring of the heterocyclyl group the maximum number of O atoms is 1, the maximum number of S atoms is 1, and the maximum total number of O and S atoms is 2. The term “heterocyclyl” is also meant to encompass partially or completely hydrogenated forms (such as dihydro, tetrahydro or perhydro forms) of the above-mentioned heteroaryl groups. Exemplary heterocyclyl groups include morpholinyl, pyrrolidinyl, imidazolidinyl, pyrazolidinyl, piperidinyl (also called piperidyl), piperazinyl, di- and tetrahydrofuranyl, di- and tetrahydrothienyl, di- and tetrahydropyranyl, urotropinyl, lactones, lactams, cyclic imides, and cyclic anhydrides. A “substituted heterocyclyl” means that one or more (such as 1 to the maximum number of hydrogen atoms bound to a heterocyclyl group, e.g., 1, 2, 3, 4, 5, 6, 7, 8, 9, or up to 10, such as between 1 to 5, 1 to 4, or 1 to 3, or 1 or 2) hydrogen atoms of the heterocyclyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein.

The expression “partially hydrogenated form” of an unsaturated compound or group as used herein means that part of the unsaturation has been removed by formally adding hydrogen to the initially unsaturated compound or group without removing all unsaturated moieties. The phrase “completely hydrogenated form” of an unsaturated compound or group is used herein interchangeably with the term “perhydro” and means that all unsaturation has been removed by formally adding hydrogen to the initially unsaturated compound or group. For example, partially hydrogenated forms of a 5-membered heteroaryl group (containing 2 double bonds in the ring, such as furan) include dihydro forms of said 5-membered heteroaryl group (such as 2,3-dihydrofuran or 2,5-dihydrofuran), whereas the tetrahydro form of said 5-membered heteroaryl group (e.g., tetrahydrofuran, i.e., THF) is a completely hydrogenated (or perhydro) form of said 5-membered heteroaryl group. Likewise, for a 6-membered heteroaryl group having 3 double bonds in the ring (such as pyridyl), partially hydrogenated forms include di- and tetrahydro forms (such as di- and tetrahydropyridyl), whereas the hexahydro form (such as piperidinyl in case of the heteroaryl pyridyl) is the completely hydrogenated (or perhydro) derivative of said 6-membered heteroaryl group. Consequently, a hexahydro form of an aryl or heteroaryl can only be considered a partially hydrogenated form according to the present disclosure if the aryl or heteroaryl contains at least 4 unsaturated moieties consisting of double and triple bonds between ring atoms.

The term “aromatic” as used in the context of hydrocarbons means that the whole molecule has to be aromatic. For example, if a monocyclic aryl is hydrogenated (either partially or completely) the resulting hydrogenated cyclic structure is classified as cycloalkyl for the purposes of the present disclosure. Likewise, if a bi- or polycyclic aryl (such as naphthyl) is hydrogenated the resulting hydrogenated bi- or polycyclic structure (such as 1,2-dihydronaphthyl) is classified as cycloalkyl for the purposes of the present disclosure (even if one ring, such as in 1,2-dihydronaphthyl, is still aromatic). A similar distinction is made within the present application between heteroaryl and heterocyclyl. For example, indolinyl, i.e., a dihydro variant of indolyl, is classified as heterocyclyl for the purposes of the present disclosure, since only one ring of the bicyclic structure is aromatic and one of the ring atoms is a heteroatom.

The term “optionally substituted” indicates that one or more (such as 1 to the maximum number of hydrogen atoms bound to a group, e.g., 1, 2, 3, 4, 5, 6, 7, 8, 9, or up to 10, such as between 1 to 5, 1 to 4, or 1 to 3, or 1 or 2) hydrogen atom(s) may be replaced with a group (i.e., a 1^{st} level substituent) different from hydrogen such as alkyl (preferably, C₁₋₆ alkyl), alkenyl (preferably, C₂₋₆ alkenyl), alkynyl (preferably, C₂₋₆ alkynyl), aryl (preferably, 6- to 14-membered aryl), heteroaryl (preferably, 3- to 14-membered heteroaryl), cycloalkyl (preferably, 3- to 14-membered cycloalkyl), heterocyclyl (preferably, 3- to 14-membered heterocyclyl), halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —NR⁷¹S(O)₁₋₂N(R⁷²)(R⁷³), —OP(O)(OR⁷¹) 2, —C(═X¹) R⁷¹, —C(═X¹)X¹R⁷¹, —X¹C(═X¹) R⁷¹, and —X¹C(═X¹)X¹R⁷¹, and/or any two 1^{st} level substituents which are bound to the same carbon atom of a cycloalkyl or heterocyclyl group may join together to form ═X¹, wherein each of the alkyl, alkenyl, alkynyl, aryl, heteroaryl, cycloalkyl, and heterocyclyl groups of the 1^{st} level substituent may themselves be substituted by one or more (e.g., one, two or three) substituents (i.e., a 2^{nd} level substituent) selected from the group consisting of C₁₋₆ alkyl, C₂₋₆ alkenyl, C₂₋₆ alkynyl, 6- to 14-membered aryl, 3- to 14-membered heteroaryl, 3- to 14-membered cycloalkyl, 3- to 14-membered heterocyclyl, halogen, —CF₃, —CN, azido, —NO₂, —OR⁸¹, —N(R⁸²)(R⁸³), —S(O)₀₋₂R⁸¹. —S(O)₁₋₂OR⁸¹, —OS(O)₁₋₂R⁸¹, —OS(O)₁₋₂OR⁸¹. —S(O)₁₋₂N(R⁸²)(R⁸³), —OS(O)₁₋₂N(R⁸²)(R⁸³), —N(R⁸¹)S(O)₁₋₂R⁸¹, —NR⁸¹S(O)₁₋₂OR⁸¹, —NR⁸¹S(O)₁₋₂N(R⁸²)(R⁸³), —OP(O)(OR⁸¹)₂, —C(═X²) R⁸¹. —C(═X²)X²R⁸¹, —X²C(═X²) R⁸¹, and —X²C(═X²)X²R⁸¹, and/or any two 2^{nd} level substituents which are bound to the same carbon atom of a cycloalkyl or heterocyclyl group being a 1^{st} level substituent may join together to form ═X², wherein each of the C₁₋₆ alkyl, C₂₋₆ alkenyl, C₂₋₆ alkynyl, 6- to 14-membered aryl, 3- to 14-membered heteroaryl, 3- to 14-membered cycloalkyl, 3- to 14-membered heterocyclyl groups of the 2^{nd} level substituent is optionally substituted with one or more (e.g., one, two or three) substituents (i.e., a 3^{rd} level substituent) independently selected from the group consisting of C₁₋₃ alkyl, halogen, —CF₃, —CN, azido, —NO₂, —OH, —O(C₁₋₃ alkyl), —OCF₃, —S(C₁₋₃ alkyl), —NH₂, —NH(C₁₋₃ alkyl), —N(C₁₋₃ alkyl)₂, —NHS(O)₂ (C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2} (C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl, and/or any two 3^{rd} level substituents which are bound to the same carbon atom of a 3- to 14-membered cycloalkyl or heterocyclyl group being a 2^{nd} level substituent may join together to form ═O, ═S, ═NH, or ═N(C₁₋₃ alkyl);
wherein
each of R⁷¹, R⁷², and R⁷³ is independently selected from the group consisting of H, C₁₋₆ alkyl, C₂₋₆ alkenyl, C₂₋₆ alkynyl, 3- to 7-membered cycloalkyl, 5- or 6-membered aryl, 5- or 6-membered heteroaryl, and 3- to 7-membered heterocyclyl, wherein each of the C₁₋₆ alkyl, C₂₋₆ alkenyl, C₂₋₆ alkynyl, 3- to 7-membered cycloalkyl. 5- or 6-membered aryl, 5- or 6-membered heteroaryl, and 3- to 7-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂ (C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2} (C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl;
each of R⁸¹, R⁸², and R⁸³ is independently selected from the group consisting of H, C₁₋₄ alkyl, C₂₋₄ alkenyl, C₂₋₄ alkynyl. 3- to 6-membered cycloalkyl. 5- or 6-membered aryl, 5- or 6-membered heteroaryl, and 3- to 6-membered heterocyclyl, wherein each of the C₁₋₄ alkyl, C₂₋₄ alkenyl, C₂₋₄ alkynyl, 3- to 6-membered cycloalkyl, 5- or 6-membered aryl, 5- or 6-membered heteroaryl, and 3- to 6-membered heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —OCF₃, ═O, —S(C₁₋₃ alkyl), —NH₂, —NH(C₁₋₃ alkyl), —N(C₁₋₃ alkyl)₂, —NHS(O)₂ (C₁₋₃ alkyl), —S(O)₂NH_{2-z}(C₁₋₃ alkyl)_{z}, —C(═O)(C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2} (C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl; and
each of X¹ and X² is independently selected from O. S. and N(R⁸⁴), wherein R⁸⁴ is H or C₁₋₃ alkyl.

Typical 1^{st} level substituents are preferably selected from the group consisting of C₁₋₆ alkyl, C₂₋₆ alkenyl, C₂₋₆ alkynyl, 6- to 14-membered (such as 6- to 10-membered) aryl, 3- to 14-membered (such as 5- or 6-membered) heteroaryl, 3- to 14-membered (such as 3- to 7-membered) cycloalkyl, 3- to 14-membered (such as 3- to 7-membered) heterocyclyl, halogen, —CN, azido, —NO₂, —OR⁷¹, —N(R⁷²)(R⁷³), —S(O)₀₋₂R⁷¹, —S(O)₁₋₂OR⁷¹, —OS(O)₁₋₂R⁷¹, —OS(O)₁₋₂OR⁷¹, —S(O)₁₋₂N(R⁷²)(R⁷³), —OS(O)₁₋₂N(R⁷²)(R⁷³), —N(R⁷¹)S(O)₁₋₂R⁷¹, —NR⁷¹S(O)₁₋₂OR⁷¹, —C(═X¹) R⁷¹, —C(═X¹)X¹R⁷¹, —X¹C(═X¹) R⁷¹, and —X¹C(═X¹)X¹R⁷¹, such as C₁₋₄ alkyl, C₂₋₄ alkenyl, C₂₋₄ alkynyl, 6-membered aryl, 5- or 6-membered heteroaryl, 3- to 7-membered cycloalkyl, 3- to 7-membered (such as 5- or 6-membered) heterocyclyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂ (C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl; wherein X¹ is independently selected from O, S, NH and N(CH₃); and each of R⁷¹, R⁷², and R⁷³ is as defined above or, preferably, is independently selected from the group consisting of H, C₁₋₄ alkyl, C₂₋₄ alkenyl, C₂₋₄ alkynyl, 5- or 6-membered cycloalkyl, 5- or 6-membered aryl, 5- or 6-membered heteroaryl, and 5- or 6-membered heterocyclyl, wherein each of the alkyl, alkenyl, alkynyl, cycloalkyl, aryl, heteroaryl, and heterocyclyl groups is optionally substituted with one, two or three substituents independently selected from the group consisting of C₁₋₃ alkyl, halogen, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂ (C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2} (C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl. In some embodiments, 1^{st} level substituents are selected from the group consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. In some embodiments, 1^{st} level substituents are selected from the group consisting of methyl, ethyl, propyl, isopropyl, halogen (such as F, Cl, or Br), and —CF₃, such as halogen (e.g., F, Cl, or Br), and —CF₃.

Typical 2^{nd} level substituents are preferably selected from the group consisting of C₁₋₄ alkyl, C₂₋₄ alkenyl, C₂₋₄ alkynyl, 6- or 10-membered aryl, 5- or 6-membered heteroaryl, 5- or 6-membered cycloalkyl, 5- or 6-membered heterocyclyl, halogen, ═O, ═S, —CF₃, —CN, azido, —NO₂, —OH, —O(C₁₋₃ alkyl), —S(C₁₋₃ alkyl), —NH₂, —NH(C₁₋₃ alkyl), —N(C₁₋₃ alkyl)₂, —NHS(O)₂ (C₁₋₃ alkyl), —S(O)₂NH_{2-z}(C₁₋₃ alkyl)_{z}, —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2} (C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl. Particular examples of 2^{nd} level substituents are independently selected from the group consisting of C₁₋₃ alkyl, phenyl, 5- or 6-membered heteroaryl, 5- or 6-membered cycloalkyl, 5- or 6-membered heterocyclyl, halogen, ═O, ═S, —CF₃, —CN, —OH, —O(C₁₋₃ alkyl), —S(C₁₋₃ alkyl), —NH₂, —NH(C₁₋₃ alkyl), —N(C₁₋₃ alkyl)₂, —NHS(O)₂ (C₁₋₃ alkyl), —C(═O)OH, —C(═O)O(C₁₋₃ alkyl), —C(═O)NH_{2-z}(C₁₋₃ alkyl)_{z}, —NHC(═O)(C₁₋₃ alkyl), —NHC(═NH)NH_{z-2}(C₁₋₃ alkyl)_{z}, and —N(C₁₋₃ alkyl)C(═NH)NH_{2-z}(C₁₋₃ alkyl)_{z}, wherein each z is independently 0, 1, or 2 and each C₁₋₃ alkyl is independently methyl, ethyl, propyl or isopropyl. Particularly preferred 2^{nd} level substituents are independently selected from the group consisting of methyl, ethyl, propyl, isopropyl, phenyl, ═O, and ═S.

Typical 3^{rd} level substituents are preferably selected from the group consisting of C₁₋₃ alkyl, phenyl, halogen, —CF₃, —OH, —OCH₃, —SCH₃, —NH_{2-z}(CH₃)_{z}, —C(═O)OH, and —C(═O)OCH₃, wherein z is 0, 1, or 2 and C₁₋₃ alkyl is methyl, ethyl, propyl or isopropyl. Particularly preferred 3^{rd} level substituents are selected from the group consisting of methyl, ethyl, propyl, isopropyl, halogen (such as F, Cl, or Br), and —CF₃, such as halogen (e.g., F, Cl, or Br), and —CF₃.

According to the present disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 151 amino acids, but the terms “peptide”, “polypeptide”, and “protein” are used herein usually as synonyms.

A “therapeutic peptide or protein” means a peptide or protein that can be used in the treatment of an individual where the expression of the peptide or protein would be of benefit, e.g., in ameliorating the symptoms of a disease. In some embodiments, the therapeutic peptide or protein has a positive or advantageous effect on a condition or disease state of a subject when provided to the subject in a therapeutically effective amount. In some embodiments, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic peptide or protein” includes entire peptides or proteins, and can also refer to therapeutically active fragments thereof. It can also include therapeutically active variants of a protein. Examples of therapeutically active proteins include, but are not limited to, antigens for vaccination and immunostimulants such as cytokines.

The term “portion” refers to a fraction. With respect to a particular structure such as an amino acid sequence or protein the term “portion” thereof may designate a continuous or a discontinuous fraction of said structure.

The terms “part” and “fragment” are used interchangeably herein and refer to a continuous element. For example, a part of a structure such as an amino acid sequence or protein refers to a continuous element of said structure. When used in context of a composition, the term “part” means a portion of the composition. For example, a part of a composition may any portion from 0.1% to 99.9% (such as 0.1%, 0.5%, 1%, 5%, 10%, 50%, 90%, or 99%) of said composition.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable, e.g., by translation of a truncated open reading frame that lacks the 3′-end of the open reading frame. A fragment shortened at the N-terminus (C-terminal fragment) is obtainable, e.g., by translation of a truncated open reading frame that lacks the 5′-end of the open reading frame, as long as the truncated open reading frame comprises a start codon that serves to initiate translation. A fragment of an amino acid sequence comprises, e.g., at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence. A fragment of an amino acid sequence comprises, e.g., a sequence of up to 8, in particular up to 10, up to 12, up to 15, up to 20, up to 30 or up to 55, consecutive amino acids of the amino acid sequence.

According to the present disclosure, a part or fragment of a peptide or protein preferably has at least one functional property of the peptide or protein from which it has been derived. Such functional properties comprise a pharmacological activity, the interaction with other peptides or proteins, an enzymatic activity, the interaction with antibodies, and the selective binding of nucleic acids. E.g., a pharmacological active fragment of a peptide or protein has at least one of the pharmacological activities of the peptide or protein from which the fragment has been derived. A part or fragment of a peptide or protein preferably comprises a sequence of at least 6, in particular at least 8, at least 10, at least 12, at least 15, at least 20, at least 30 or at least 50, consecutive amino acids of the peptide or protein. A part or fragment of a peptide or protein preferably comprises a sequence of up to 8, in particular up to 10, up to 12, up to 15, up to 20, up to 30 or up to 55, consecutive amino acids of the peptide or protein.

By “variant” as used herein and with reference to an amino acid sequence (peptide or protein) is meant an amino acid sequence that differs from a parent amino acid sequence by virtue of at least one amino acid (e.g., a different amino acid, or a modification of the same amino acid). The parent amino acid sequence may be a naturally occurring or wild type (WT) amino acid sequence, or may be a modified version of a wild type amino acid sequence. In some embodiments, the variant amino acid sequence has at least one amino acid difference compared to the parent amino acid sequence, e.g., from 1 to about 20 amino acid differences, and preferably from 1 to about 10 or from 1 to about 5 amino acid differences compared to the parent.

By “wild type” or “WT” or “native” herein is meant an amino acid sequence that is found in nature, including allelic variations. A wild type amino acid sequence, peptide or protein has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all mutants, splice variants, posttranslationally modified variants, conformations, isoforms, allelic variants, species variants, and species homologs, in particular those which are naturally occurring. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In some embodiments, conservative amino acid substitutions include substitutions within the following groups:
glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, in some embodiments, continuous amino acids. In some embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences. “Sequence identity” between two nucleic acid sequences indicates the percentage of nucleotides that are identical between the sequences.

The terms “% identical” and “% identity” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison”, in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N and TFASTA in Wisconsin Genetics Software Package, Genetics Computer Group, 575 Science Drive, Madison, Wis.). In some embodiments, percent identity of two sequences is determined using the BLASTN or BLASTP algorithm, as available on the United States National Center for Biotechnology Information (NCBI) website (e.g., at blast.ncbi.nlm.nih.gov/Blast.cgi?PAGE_TYPE=BlastSearch&BLAST_SPEC=blast2seq&LINK_LOC=align2seq). In some embodiments, the algorithm parameters used for BLASTN algorithm on the NCBI website include: (i) Expect Threshold set to 10; (ii) Word Size set to 28; (iii) Max matches in a query range set to 0; (iv) Match/Mismatch Scores set to 1, -2; (v) Gap Costs set to Linear; and (vi) the filter for low complexity regions being used. In some embodiments, the algorithm parameters used for BLASTP algorithm on the NCBI website include: (i) Expect Threshold set to 10; (ii) Word Size set to 3; (iii) Max matches in a query range set to 0; (iv) Matrix set to BLOSUM62; (v) Gap Costs set to Existence: 11 Extension: 1; and (vi) conditional compositional score matrix adjustment.

Percentage identity is obtained by determining the number of identical positions at which the sequences to be compared correspond, dividing this number by the number of positions compared (e.g., the number of positions in the reference sequence) and multiplying this result by 100.

In some embodiments, the degree of similarity or identity is given for a region which is at least about 50%, at least about 60%, at least about 70%, at least about 80%, at least about 90% or about 100% of the entire length of the reference sequence. For example, if the reference nucleic acid sequence consists of 200 nucleotides, the degree of identity is given for at least about 100, at least about 120, at least about 140, at least about 160, at least about 180, or about 200 nucleotides, in some embodiments continuous nucleotides. In some embodiments, the degree of similarity or identity is given for the entire length of the reference sequence.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In some embodiments, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to antigens or antigenic sequences, one particular function is one or more immunogenic activities displayed by the amino acid sequence from which the fragment or variant is derived. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., inducing an immune response. In some embodiments, the modifications in the amino acid sequence of the parent molecule or sequence do not significantly affect or alter the characteristics of the molecule or sequence.

In different embodiments, the function of the functional fragment or functional variant may be reduced but still significantly present, e.g., immunogenicity of the functional variant may be at least 50%, at least 60%, at least 70%, at least 80%, or at least 90% of the parent molecule or sequence. However, in other embodiments, immunogenicity of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

In some embodiments, “isolated” means altered or removed (e.g., purified) from the natural state or from an artificial composition, such as a composition from a production process. For example, a nucleic acid (such as RNA), peptide or polypeptide naturally present in a living animal is not “isolated”, but the same nucleic acid, peptide or polypeptide partially or completely separated from the coexisting materials of its natural state is “isolated”. An isolated nucleic acid, peptide or polypeptide can exist in substantially purified form, or can exist in a non-native environment such as, for example, a host cell. In certain embodiments, the nucleic acid (such as DNA or RNA, in particular mRNA) used in the present disclosure is in substantially purified form. In some embodiments, a solution (preferably an aqueous solution) of nucleic acid (such as DNA or RNA, in particular mRNA) in substantially purified form contains a first buffer system.

The term “genetic modification” or simply “modification” includes the transfection of cells with nucleic acid.

The term “transfection” relates to the introduction of nucleic acids, in particular RNA, into a cell. For purposes of the present disclosure, the term “transfection” also includes the introduction of a nucleic acid into a cell or the uptake of a nucleic acid by such cell, wherein the cell may be present in a subject, e.g., a patient. Thus, according to the present disclosure, a cell for transfection of a nucleic acid described herein can be present in vitro or in vivo, e.g. the cell can form part of an organ, a tissue and/or an organism of a patient. According to the disclosure, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, nucleic acid encoding antigen is transiently transfected into cells. RNA can be transfected into cells to transiently express its coded protein.

The disclosure includes analogs of a peptide, polypeptide, or protein. According to the present disclosure, an analog of a peptide, polypeptide, or protein is a modified form of said peptide, polypeptide, or protein from which it has been derived and has at least one functional property of said peptide, polypeptide, or protein. E.g., a pharmacological active analog of a peptide, polypeptide, or protein has at least one of the pharmacological activities of the peptide, polypeptide, or protein from which the analog has been derived. Such modifications include any chemical modification and comprise single or multiple substitutions, deletions and/or additions of any molecules associated with the peptide, polypeptide, or protein, such as carbohydrates, lipids and/or proteins or peptides. In some embodiments, “analogs” of peptides, polypeptides, or proteins include those modified forms resulting from glycosylation, acetylation, phosphorylation, amidation, palmitoylation, myristoylation, isoprenylation, lipidation, alkylation, derivatization, introduction of protective/blocking groups, proteolytic cleavage or binding to an antibody or to another cellular ligand. The term “analog” also extends to all functional chemical equivalents of said peptides, polypeptides, or proteins.

As used herein, the terms “linked”, “fused”, or “fusion” are used interchangeably. These terms refer to the joining together of two or more elements or components or domains.

According to the present disclosure, it is preferred that a nucleic acid such as DNA or RNA (e.g., mRNA) encoding a peptide, polypeptide, or protein once taken up by or introduced, i.e. transfected or transduced, into a cell which cell may be present in vitro or in a subject results in expression of said peptide, polypeptide, or protein. The cell may express the encoded peptide, polypeptide, or protein intracellularly (e.g. in the cytoplasm and/or in the nucleus), may secrete the encoded peptide, polypeptide, or protein, or may express it on the surface.

According to the present disclosure, terms such as “nucleic acid expressing” and “nucleic acid encoding” or similar terms (such as “RNA encoding”) are used interchangeably herein and with respect to a particular peptide, polypeptide, or protein mean that the nucleic acid, if present in the appropriate environment, preferably within a cell, can be expressed to produce said peptide, polypeptide, or protein.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “expansion” refers to a process wherein a specific entity is multiplied. In the context of the present disclosure, the term is preferably used in the context of an immunological response in which immune effector cells are stimulated by an antigen, proliferate, and the specific immune effector cell recognizing said antigen is amplified. In some embodiments, expansion leads to differentiation of the immune effector cells.

An “antigen” according to the present disclosure covers any substance that will elicit an immune response and/or any substance against which an immune response or an immune mechanism such as a cellular response is directed. This also includes situations wherein the antigen is processed into antigen peptides and an immune response or an immune mechanism is directed against one or more antigen peptides, in particular if presented in the context of MHC molecules. In particular, an “antigen” relates to any substance, preferably a peptide or protein, that reacts specifically with antibodies or T-lymphocytes (T-cells). According to the present disclosure, the term “antigen” comprises any molecule which comprises at least one epitope, such as a T cell epitope. In certain embodiments, an antigen in the context of the present disclosure is a molecule which, optionally after processing, induces an immune reaction, which may be specific for the antigen (including cells expressing the antigen). In some embodiments, an antigen is a disease-associated antigen, such as a tumor antigen, a viral antigen, or a bacterial antigen, or an epitope derived from such antigen.

In some embodiments, an antigen is presented or present on the surface of cells of the immune system such as antigen presenting cells like dendritic cells or macrophages. An antigen or a procession product thereof such as a T cell epitope is in some embodiments bound by an antigen receptor. Accordingly, an antigen or a procession product thereof may react specifically with immune effector cells such as T-lymphocytes (T cells).

According to the present disclosure, any suitable antigen may be used, which is a candidate for an immune response, wherein the immune response may be both a humoral as well as a cellular immune response. In the context of some embodiments of the present disclosure, the antigen is preferably presented by a cell, preferably by an antigen presenting cell, in the context of MHC molecules, which results in an immune response against the antigen. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present disclosure, an antigen may correspond to a naturally occurring product, for example, a viral protein, or a part thereof.

The term “disease-associated antigen” is used in its broadest sense to refer to any antigen associated with a disease. A disease-associated antigen is a molecule which contains epitopes that will stimulate a host's immune system to make a cellular antigen-specific immune response and/or a humoral antibody response against the disease. Disease-associated antigens include pathogen-associated antigens, i.e., antigens which are associated with infection by microbes, typically microbial antigens (such as bacterial or viral antigens), or antigens associated with cancer, typically tumors, such as tumor antigens.

In certain embodiments, the antigen is a tumor antigen, i.e., a part of a tumor cell, in particular those which primarily occur intracellularly or as surface antigens of tumor cells. In some embodiments, the antigen is a pathogen-associated antigen, i.e., an antigen derived from a pathogen, e.g., from a virus, bacterium, unicellular organism, or parasite, for example a viral antigen such as viral ribonucleoprotein or coat protein. In particular, the antigen should be presented by MHC molecules which results in modulation, in particular activation of cells of the immune system, preferably CD4+ and CD8+ lymphocytes, in particular via the modulation of the activity of a T-cell receptor.

The term “tumor antigen” or “tumor-associated antigen” refers to a constituent of cancer cells which may be derived from the cytoplasm, the cell surface or the cell nucleus. In particular, it refers to those antigens which are produced intracellularly or as surface antigens on tumor cells. For example, tumor antigens include the carcinoembryonal antigen, al-fetoprotein, isoferritin, and fetal sulphoglycoprotein, α2-H-ferroprotein and γ-fetoprotein, as well as various virus tumor antigens. According to the present disclosure, a tumor antigen preferably comprises any antigen which is characteristic for tumors or cancers as well as for tumor or cancer cells with respect to type and/or expression level.

The term “viral antigen” refers to any viral component having antigenic properties, i.e., being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “epitope” refers to an antigenic determinant in a molecule such as an antigen, i.e., to a part in or fragment of the molecule that is recognized by the immune system, for example, that is recognized by antibodies, T cells or B cells, in particular when presented in the context of MHC molecules. An epitope of a protein preferably comprises a continuous or discontinuous portion of said protein and is preferably between about 5 and about 100, preferably between about 5 and about 50, more preferably between about 8 and about 0, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. It is particularly preferred that the epitope in the context of the present disclosure is a T cell epitope.

Terms such as “epitope”, “fragment of an antigen”, “immunogenic peptide” and “antigen peptide” are used interchangeably herein and preferably relate to an incomplete representation of an antigen which is preferably capable of eliciting an immune response against the antigen or a cell expressing or comprising and preferably presenting the antigen. Preferably, the terms relate to an immunogenic portion of an antigen. Preferably, it is a portion of an antigen that is recognized (i.e., specifically bound) by a T cell receptor, in particular if presented in the context of MHC molecules. Certain preferred immunogenic portions bind to an MHC class I or class II molecule. The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about 5 and about 100, such as between about 5 and about 50, more preferably between about 8 and about 30, most preferably between about 8 and about 25 amino acids in length, for example, the epitope may be preferably 9, 10, 11, 12, 13, 14, 15, 16, 17, 18, 19, 20, 21, 22, 23, 24, or 25 amino acids in length. In some embodiments, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

The peptide and protein antigen can be 2 to 100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

The peptide or protein antigen can be any peptide or protein that can induce or increase the ability of the immune system to develop antibodies and T cell responses to the peptide or protein.

In some embodiments, vaccine antigen, i.e., an antigen whose inoculation into a subject induces an immune response, is recognized by an immune effector cell. Preferably, the vaccine antigen if recognized by an immune effector cell is able to induce in the presence of appropriate co-stimulatory signals, stimulation, priming and/or expansion of the immune effector cell carrying an antigen receptor recognizing the vaccine antigen. In the context of the embodiments of the present disclosure, the vaccine antigen is preferably presented or present on the surface of a cell, preferably an antigen presenting cell.

In some embodiments, an antigen is expressed in a diseased cell (such as tumor cell or an infected cell).

In some embodiments, an antigen is presented by a diseased cell (such as tumor cell or an infected cell). In some embodiments, an antigen receptor is a TCR which binds to an epitope of an antigen presented in the context of MHC. In some embodiments, binding of a TCR when expressed by T cells and/or present on T cells to an antigen presented by cells such as antigen presenting cells results in stimulation, priming and/or expansion of said T cells. In some embodiments, binding of a TCR when expressed by T cells and/or present on T cells to an antigen presented on diseased cells results in cytolysis and/or apoptosis of the diseased cells, wherein said T cells preferably release cytotoxic factors, e.g., perforins and granzymes.

In some embodiments, an antigen is expressed on the surface of a diseased cell (such as tumor cell or an infected cell). In some embodiments, an antigen receptor is a CAR which binds to an extracellular domain or to an epitope in an extracellular domain of an antigen. In some embodiments, a CAR binds to native epitopes of an antigen present on the surface of living cells. In some embodiments, binding of a CAR when expressed by T cells and/or present on T cells to an antigen present on cells such as antigen presenting cells results in stimulation, priming and/or expansion of said T cells. In some embodiments, binding of a CAR when expressed by T cells and/or present on T cells to an antigen present on diseased cells results in cytolysis and/or apoptosis of the diseased cells, wherein said T cells preferably release cytotoxic factors, e.g., perforins and granzymes.

In some embodiments, an antigen receptor is an antibody or B cell receptor which binds to an epitope in an antigen. In some embodiments, an antibody or B cell receptor binds to native epitopes of an antigen.

The term “expressed on the cell surface” or “associated with the cell surface” means that a molecule such as an antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules. An antigen is expressed on the surface of cells if it is located at the surface of said cells and is accessible to binding by, e.g., antigen-specific antibodies added to the cells. In some embodiments, an antigen expressed on the surface of cells is an integral membrane protein having an extracellular portion which may be recognized by a CAR.

The term “extracellular portion” or “exodomain” in the context of the present disclosure refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted, in particular when presented on the surface of antigen presenting cells or diseased cells such as cancer cells in the context of MHC molecules and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as interferon-γ) can be measured. In certain embodiments of the present disclosure, the nucleic acid (such as DNA or RNA, in particular mRNA) encodes at least one epitope.

The term “target” shall mean an agent such as a cell or tissue which is a target for an immune response such as a cellular immune response. Targets include cells that present an antigen or an antigen epitope, i.e., a peptide fragment derived from an antigen. In som embodiments, the target cell is a cell expressing an antigen and preferably presenting said antigen with class I MHC.

“Antigen processing” refers to the degradation of an antigen into processing products which are fragments of said antigen (e.g., the degradation of a protein into peptides) and the association of one or more of these fragments (e.g., via binding) with MHC molecules for presentation by cells, preferably antigen-presenting cells to specific T-cells. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naive T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

The term “dendritic cell” (DC) refers to a subtype of phagocytic cells belonging to the class of antigen presenting cells. In some embodiments, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In some embodiments, the dendritic cells are splenic dendritic cells.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In some embodiments, the macrophages are splenic macrophages.

By “antigen-responsive CTL” is meant a CD8+ T-cell that is responsive to an antigen or a peptide derived from said antigen, which is presented with class I MHC on the surface of antigen presenting cells.

According to the disclosure, CTL responsiveness may include sustained calcium flux, cell division, production of cytokines such as IFN-γ and TNF-α, up-regulation of activation markers such as CD44 and CD69, and specific cytolytic killing of tumor antigen expressing target cells. CTL responsiveness may also be determined using an artificial reporter that accurately indicates CTL responsiveness.

The terms “immune response” and “immune reaction” are used herein interchangeably in their conventional meaning and refer to an integrated bodily response to an antigen and preferably refers to a cellular immune response, a humoral immune response, or both. According to the disclosure, the term “immune response to” or “immune response against” with respect to an agent such as an antigen, cell or tissue, relates to an immune response such as a cellular response directed against the agent. An immune response may comprise one or more reactions selected from the group consisting of developing antibodies against one or more antigens and expansion of antigen-specific T-lymphocytes, preferably CD4+ and CD8+T-lymphocytes, more preferably CD8+T-lymphocytes, which may be detected in various proliferation or cytokine production tests in vitro.

The terms “inducing an immune response” and “eliciting an immune response” and similar terms in the context of the present disclosure refer to the induction of an immune response, preferably the induction of a cellular immune response, a humoral immune response, or both. The immune response may be protective/preventive/prophylactic and/or therapeutic. The immune response may be directed against any immunogen or antigen or antigen peptide, preferably against a tumor-associated antigen or a pathogen-associated antigen (e.g., an antigen of a virus (such as influenza virus (A, B, or C), CMV or

RSV)). “Inducing” in this context may mean that there was no immune response against a particular antigen or pathogen before induction, but it may also mean that there was a certain level of immune response against a particular antigen or pathogen before induction and after induction said immune response is enhanced. Thus, “inducing the immune response” in this context also includes “enhancing the immune response”. Preferably, after inducing an immune response in an individual, said individual is protected from developing a disease such as an infectious disease or a cancerous disease or the disease condition is ameliorated by inducing an immune response.

The terms “cellular immune response”, “cellular response”, “cell-mediated immunity” or similar terms are meant to include a cellular response directed to cells characterized by expression of an antigen and/or presentation of an antigen with class I or class II MHC. The cellular response relates to cells called T cells or T lymphocytes which act as either “helpers” or “killers”. The helper T cells (also termed CD4+T cells) play a central role by regulating the immune response and the killer cells (also termed cytotoxic T cells, cytolytic T cells, CD8+ T cells or CTLs) kill cells such as diseased cells.

The term “humoral immune response” refers to a process in living organisms wherein antibodies are produced in response to agents and organisms, which they ultimately neutralize and/or eliminate. The specificity of the antibody response is mediated by T and/or B cells through membrane-associated receptors that bind antigen of a single specificity. Following binding of an appropriate antigen and receipt of various other activating signals, B lymphocytes divide, which produces memory B cells as well as antibody secreting plasma cell clones, each producing antibodies that recognize the identical antigenic epitope as was recognized by its antigen receptor. Memory B lymphocytes remain dormant until they are subsequently activated by their specific antigen. These lymphocytes provide the cellular basis of memory and the resulting escalation in antibody response when re-exposed to a specific antigen.

The term “antibody” as used herein, refers to an immunoglobulin molecule, which is able to specifically bind to an epitope on an antigen. In particular, the term “antibody” refers to a glycoprotein comprising at least two heavy (H) chains and two light (L) chains inter-connected by disulfide bonds. The term “antibody” includes monoclonal antibodies, recombinant antibodies, human antibodies, humanized antibodies, chimeric antibodies and combinations of any of the foregoing. Each heavy chain is comprised of a heavy chain variable region (VH) and a heavy chain constant region (CH). Each light chain is comprised of a light chain variable region (VL) and a light chain constant region (CL). The variable regions and constant regions are also referred to herein as variable domains and constant domains, respectively. The VH and VL regions can be further subdivided into regions of hypervariability, termed complementarity determining regions (CDRs), interspersed with regions that are more conserved, termed framework regions (FRs). Each VH and VL is composed of three CDRs and four FRs, arranged from amino-terminus to carboxy-terminus in the following order: FR1, CDR1, FR2, CDR2, FR3, CDR3, FR4. The CDRs of a VH are termed HCDR1, HCDR2 and HCDR3, the CDRs of a VL are termed LCDR1, LCDR2 and LCDR3. The variable regions of the heavy and light chains contain a binding domain that interacts with an antigen. The constant regions of an antibody comprise the heavy chain constant region (CH) and the light chain constant region (CL), wherein CH can be further subdivided into constant domain CH₁, a hinge region, and constant domains CH₂ and CH₃ (arranged from amino-terminus to carboxy-terminus in the following order: CH₁, CH₂, CH₃). The constant regions of the antibodies may mediate the binding of the immunoglobulin to host tissues or factors, including various cells of the immune system (e.g., effector cells) and the first component (Clq) of the classical complement system. Antibodies can be intact immunoglobulins derived from natural sources or from recombinant sources and can be immunoactive portions of intact immunoglobulins. Antibodies are typically tetramers of immunoglobulin molecules. Antibodies may exist in a variety of forms including, for example, polyclonal antibodies, monoclonal antibodies, Fv, Fab and F (ab) 2, as well as single chain antibodies and humanized antibodies.

The term “immunoglobulin” relates to proteins of the immunoglobulin superfamily, preferably to antigen receptors such as antibodies or the B cell receptor (BCR). The immunoglobulins are characterized by a structural domain, i.e., the immunoglobulin domain, having a characteristic immunoglobulin (Ig) fold. The term encompasses membrane bound immunoglobulins as well as soluble immunoglobulins. Membrane bound immunoglobulins are also termed surface immunoglobulins or membrane immunoglobulins, which are generally part of the BCR. Soluble immunoglobulins are generally termed antibodies. Immunoglobulins generally comprise several chains, typically two identical heavy chains and two identical light chains which are linked via disulfide bonds. These chains are primarily composed of immunoglobulin domains, such as the V_{L} (variable light chain) domain, C_{L} (constant light chain) domain, V_{H} (variable heavy chain) domain, and the C_{H} (constant heavy chain) domains C_{H}1, C_{H}2, C_{H}3, and C_{H}4. There are five types of mammalian immunoglobulin heavy chains, i.e., α, δ, ε, γ, and μ which account for the different classes of antibodies, i.e., IgA, IgD, IgE, IgG, and IgM. As opposed to the heavy chains of soluble immunoglobulins, the heavy chains of membrane or surface immunoglobulins comprise a transmembrane domain and a short cytoplasmic domain at their carboxy-terminus. In mammals there are two types of light chains, i.e., lambda and kappa. The immunoglobulin chains comprise a variable region and a constant region. The constant region is essentially conserved within the different isotypes of the immunoglobulins, wherein the variable part is highly divers and accounts for antigen recognition.

The terms “vaccination” and “immunization” describe the process of treating an individual for therapeutic or prophylactic reasons and relate to the procedure of administering one or more immunogen(s) or antigen(s) or derivatives thereof, in particular in the form of DNA or RNA (especially mRNA) coding therefor, as described herein to an individual and stimulating an immune response against said one or more immunogen(s) or antigen(s) or cells characterized by presentation of said one or more immunogen(s) or antigen(s).

By “cell characterized by presentation of an antigen” or “cell presenting an antigen” or “MHC molecules which present an antigen on the surface of an antigen presenting cell” or similar expressions is meant a cell such as a diseased cell, in particular a tumor cell or an infected cell, or an antigen presenting cell presenting the antigen or an antigen peptide, either directly or following processing, in the context of MHC molecules, preferably MHC class I and/or MHC class II molecules, most preferably MHC class I molecules.

In the context of the present disclosure, the term “transcription” relates to a process, wherein the genetic code in a DNA sequence is transcribed into RNA (especially mRNA). Subsequently, the RNA (especially mRNA) may be translated into peptide, polypeptide, or protein.

The term “expression” as used herein includes the transcription and/or translation of a particular nucleotide sequence.

With respect to RNA, the term “expression” or “translation” relates to the process in the ribosomes of a cell by which a strand of mRNA directs the assembly of a sequence of amino acids to make a peptide, polypeptide, or protein.

In the context of the present disclosure, the term “RNA encodes” means that the RNA, if present in the appropriate environment, such as within cells of a target tissue, can direct the assembly of amino acids to produce the peptide or protein it encodes during the process of translation.

The term “optional” or “optionally” as used herein means that the subsequently described event, circumstance or condition may or may not occur, and that the description includes instances where said event, circumstance, or condition occurs and instances in which it does not occur.

A medical preparation, in particular kit, described herein may comprise instructional material or instructions. As used herein, “instructional material” or “instructions” includes a publication, a recording, a diagram, or any other medium of expression which can be used to communicate the usefulness of the compositions and methods of the present disclosure. The instructional material of the kit of the present disclosure may, for example, be affixed to a container which contains the compositions/formulations of the present disclosure or be shipped together with a container which contains the compositions/formulations. Alternatively, the instructional material may be shipped separately from the container with the intention that the instructional material and the compositions be used cooperatively by the recipient.

Prodrugs of a particular compound described herein are those compounds that upon administration to an individual undergo chemical conversion under physiological conditions to provide the particular compound. Additionally, prodrugs can be converted to the particular compound by chemical or biochemical methods in an ex vivo environment. For example, prodrugs can be slowly converted to the particular compound when, for example, placed in a transdermal patch reservoir with a suitable enzyme or chemical reagent. Exemplary prodrugs are esters (using an alcohol or a carboxy group contained in the particular compound) or amides (using an amino or a carboxy group contained in the particular compound) which are hydrolyzable in vivo. Specifically, any amino group which is contained in the particular compound and which bears at least one hydrogen atom can be converted into a prodrug form. Typical N-prodrug forms include carbamates, Mannich bases, enamines, and enaminones.

In the present specification, a structural formula of a compound may represent a certain isomer of said compound. It is to be understood, however, that the present disclosure includes all isomers such as geometrical isomers, optical isomers based on an asymmetrical carbon, stereoisomers, tautomers and the like which occur structurally and isomer mixtures and is not limited to the description of the formula. Furthermore, in the present specification, a structural formula of a compound may represent a specific salt and/or solvate of said compound. It is to be understood, however, that the present disclosure includes all salts (e.g., pharmaceutically acceptable salts) and solvates (e.g., hydrates) and is not limited to the description of the specific salt and/or solvate.

“Isomers” are compounds having the same molecular formula but differ in structure (“structural isomers”) or in the geometrical (spatial) positioning of the functional groups and/or atoms (“stereoisomers”). “Enantiomers” are a pair of stereoisomers which are non-superimposable mirror-images of each other. A “racemic mixture” or “racemate” contains a pair of enantiomers in equal amounts and is denoted by the prefix (+). “Diastereomers” are stereoisomers which are non-superimposable and which are not mirror-images of each other. “Tautomers” are structural isomers of the same chemical substance that spontaneously and reversibly interconvert into each other, even when pure, due to the migration of individual atoms or groups of atoms; i.e., the tautomers are in a dynamic chemical equilibrium with each other. An example of tautomers are the isomers of the keto-enol-tautomerism. “Conformers” are stereoisomers that can be interconverted just by rotations about formally single bonds, and include—in particular—those leading to different 3-dimentional forms of (hetero) cyclic rings, such as chair, half-chair, boat, and twist-boat forms of cyclohexane.

The term “solvate” as used herein refers to an addition complex of a dissolved material in a solvent (such as an organic solvent (e.g., an aliphatic alcohol (such as methanol, ethanol, n-propanol, isopropanol), acetone, acetonitrile, ether, and the like), water or a mixture of two or more of these liquids), wherein the addition complex exists in the form of a crystal or mixed crystal. The amount of solvent contained in the addition complex may be stoichiometric or non-stoichiometric. A “hydrate” is a solvate, wherein the solvent is water.

In isotopically labeled compounds one or more atoms are replaced by a corresponding atom having the same number of protons but differing in the number of neutrons. For example, a hydrogen atom may be replaced by a deuterium or tritium atom. Exemplary isotopes which can be used in the present disclosure include deuterium, tritium, ¹¹C, ¹³C, ¹⁴C, ¹⁵N, ¹⁸F, ³²P, ³²S, ³⁵S, ³⁶Cl, and ¹²⁵I.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PDI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

In some embodiments, the “polydispersity index” is calculated based on dynamic light scattering measurements by the so-called cumulant analysis as mentioned in the definition of the “average diameter”. Under certain prerequisites, it can be taken as a measure of the size distribution of an ensemble of nanoparticles.

The “radius of gyration” (abbreviated herein as R_{g}) of a particle about an axis of rotation is the radial distance of a point from the axis of rotation at which, if the whole mass of the particle is assumed to be concentrated, its moment of inertia about the given axis would be the same as with its actual distribution of mass. Mathematically, R_{g} is the root mean square distance of the particle's components from either its center of mass or a given axis. For example, for a macromolecule composed of n mass elements, of masses mᵢ (i=1, 2, 3, . . . , n), located at fixed distances sᵢ from the center of mass, R_{g} is the square-root of the mass average of sᵢ² over all mass elements and can be calculated as follows:

$${R}_{g} = {( {{\sum }_{i = 1}}^{n} {m}_{i} \cdot {s}_{i}^{2} / {{\sum }_{i = 1}}^{n} {m}_{i} )}^{1 / 2}$$

The radius of gyration can be determined or calculated experimentally, e.g., by using light scattering. In particular, for small scattering vectors q the structure function S is defined as follows:

$$S ( \vec{q} ) \approx N \cdot ( 1 - \frac{{q}^{2} \cdot {R}_{g}^{2}}{3} )$$

wherein N is the number of components (Guinier's law).

The “D10 value”, in particular regarding a quantitative size distribution of particles, is the diameter at which 10% of the particles have a diameter less than this value. The D10 value is a means to describe the proportion of the smallest particles within a population of particles (such as within a particle peak obtained from a field-flow fractionation).

“D50 value”, in particular regarding a quantitative size distribution of particles, is the diameter at which 50% of the particles have a diameter less than this value. The D50 value is a means to describe the mean particle size of a population of particles (such as within a particle peak obtained from a field-flow fractionation).

The “D90 value”, in particular regarding a quantitative size distribution of particles, is the diameter at which 90% of the particles have a diameter less than this value. The “D95”, “D99”, and “D100” values have corresponding meanings. The D90, D95, D99, and D100 values are means to describe the proportion of the larger particles within a population of particles (such as within a particle peak obtained from a field-flow fractionation).

The “hydrodynamic radius” (which is sometimes called “Stokes radius” or “Stokes-Einstein radius”) of a particle is the radius of a hypothetical hard sphere that diffuses at the same rate as said particle. The hydrodynamic radius is related to the mobility of the particle, taking into account not only size but also solvent effects. For example, a smaller charged particle with stronger hydration may have a greater hydrodynamic radius than a larger charged particle with weaker hydration. This is because the smaller particle drags a greater number of water molecules with it as it moves through the solution. Since the actual dimensions of the particle in a solvent are not directly measurable, the hydrodynamic radius may be defined by the Stokes-Einstein equation:

$${R}_{h} = \frac{{k}_{B} \cdot T}{6 \cdot π \cdot η \cdot D}$$

wherein k_{B} is the Boltzmann constant; Tis the temperature; n is the viscosity of the solvent; and D is the diffusion coefficient. The diffusion coefficient can be determined experimentally, e.g., by using dynamic light scattering (DLS). Thus, one procedure to determine the hydrodynamic radius of a particle or a population of particles (such as the hydrodynamic radius of particles such as LNPs contained in a formulation or composition as disclosed herein or the hydrodynamic radius of a particle peak obtained from subjecting such a formulation or composition to field-flow fractionation) is to measure the DLS signal of said particle or population of particles (such as DLS signal of particles such as LNPs contained in a formulation or composition as disclosed herein or the DLS signal of a particle peak obtained from subjecting such a formulation or composition to field-flow fractionation).

The term “aggregate” as used herein relates to a cluster of particles, wherein the particles are identical or very similar and adhere to each other in a non-covalently manner (e.g., via ionic interactions, H bridge interactions, dipole interactions, and/or van der Waals interactions).

The expression “light scattering” as used herein refers to the physical process where light is forced to deviate from a straight trajectory by one or more paths due to localized non-uniformities in the medium through which the light passes.

The term “UV” means ultraviolet and designates a band of the electromagnetic spectrum with a wavelength from 10 nm to 400 nm, i.e., shorter than that of visible light but longer than X-rays.

The expression “multi-angle light scattering” or “MALS” as used herein relates to a technique for measuring the light scattered by a sample into a plurality of angles. “Multi-angle” means in this respect that scattered light can be detected at different discrete angles as measured, for example, by a single detector moved over a range including the specific angles selected or an array of detectors fixed at specific angular locations. In certain embodiments, the light source used in MALS is a laser source (MALLS: multi-angle laser light scattering). Based on the MALS signal of a composition comprising particles and by using an appropriate formalism (e.g., Zimm plot, Berry plot, or Debye plot), it is possible to determine the radius of gyration (R_{g}) and, thus, the size of said particles. Preferably, the Zimm plot is a graphical presentation using the following equation (or the reciprocal thereof):

$$\frac{{R}_{θ}}{{K}^{*} c} = {M}_{w} P ( θ ) - 2 {A}_{2} {cM}_{w}^{2} {P}^{2} ( θ )$$

wherein c is the mass concentration of the particles in the solvent (g/mL); A₂ is the second virial coefficient (mol·mL/g²); P(θ) is a form factor relating to the dependence of scattered light intensity on angle; R_{θ} is the excess Rayleigh ratio (cm⁻¹); and K* is an optical constant that is equal to 4π²ηₒ (dn/dc)²λ₀⁴N_{A}⁻¹, where no is the refractive index of the solvent at the incident radiation (vacuum) wavelength, λ₀ is the incident radiation (vacuum) wavelength (nm), N_{A} is Avogadro's number (mol-1), and dn/dc is the differential refractive index increment (mL/g) (cf., e.g., Buchholz et al. (Electrophoresis 22 (2001), 4118-4128); B. H. Zimm (J. Chem. Phys. 13 (1945), 141; P. Debye (J. Appl. Phys. 15 (1944): 338; and W. Burchard (Anal. Chem. 75 (2003), 4279-4291). Preferably, the Berry plot is calculated the following term or the reciprocal thereof:

$$\sqrt{\frac{{R}_{θ}}{{K}^{*} c}}$$

wherein c, R_{θ} and K* are as defined above. Preferably, the Debye plot is calculated the following term or the reciprocal thereof:

$$\frac{{K}^{*} c}{{R}_{θ}}$$

wherein c, R_{θ} and K* are as defined above.

The expression “dynamic light scattering” or “DLS” as used herein refers to a technique to determine the size and size distribution profile of particles, in particular with respect to the hydrodynamic radius of the particles. A monochromatic light source, usually a laser, is shot through a polarizer and into a sample. The scattered light then goes through a second polarizer where it is detected and the resulting image is projected onto a screen. The particles in the solution are being hit with the light and diffract the light in all directions. The diffracted light from the particles can either interfere constructively (light regions) or destructively (dark regions). This process is repeated at short time intervals and the resulting set of speckle patterns are analyzed by an autocorrelator that compares the intensity of light at each spot over time.

The expression “static light scattering” or “SLS” as used herein refers to a technique to determine the size and size distribution profile of particles, in particular with respect to the radius of gyration of the particles, and/or the molar mass of particles. A high-intensity monochromatic light, usually a laser, is launched in a solution containing the particles. One or many detectors are used to measure the scattering intensity at one or many angles. The angular dependence is needed to obtain accurate measurements of both molar mass and size for all macromolecules of radius. Hence simultaneous measurements at several angles relative to the direction of incident light, known as multi-angle light scattering (MALS) or multi-angle laser light scattering (MALLS), is generally regarded as the standard implementation of static light scattering.

“Immunogenicity” is the ability of a foreign substance, such as RNA, to provoke an immune response in the body of a human or other animal. The innate immune system is the component of the immune system that is relatively unspecific and immediate. It is one of two main components of the vertebrate immune system, along with the adaptive immune system.

As used herein “endogenous” refers to any material from or produced inside an organism, cell, tissue or system.

As used herein, the term “exogenous” refers to any material introduced from or produced outside an organism, cell, tissue or system.

As used herein, the terms “linked”, “fused”, or “fusion” are used interchangeably. These terms refer to the joining together of two or more elements or components or domains.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

In the context of the present disclosure, the term “nucleic acid particle” relates to a particle that contains nucleiac acid (such as DNA or RNA). Thus, the term “RNA particle” relates to a particle that contains RNA.

A “polymer,” as used herein, is given its ordinary meaning, i.e., a molecular structure comprising one or more repeating units (monomers), connected by covalent bonds. The repeating units can all be identical, or in some cases, there can be more than one type of repeating unit present within the polymer. In some cases, the polymer is biologically derived, i.e., a biopolymer such as a protein. In some cases, additional moieties can also be present in the polymer, for example targeting moieties such as those described herein.

The term “repeating unit” relates to an elementary unit which periodically repeats itself along the polymeric chain of a polymer and which is derived from one monomer. Although the structures of the repeating unit and its corresponding monomer are often coincident, they may differ from each other.

If more than one type of repeating unit is present within the polymer, then the polymer is said to be a “copolymer.” It is to be understood that in any embodiment employing a polymer, the polymer being employed can be a copolymer in some cases. The repeating units forming the copolymer can be arranged in any fashion. For example, the repeating units can be arranged in a random manner, in a periodic manner, in an alternating manner, or in a block wise manner. In this respect the term “arranged in a random manner” means that the sequence of repeating units in the copolymer follows a statistical rule (such copolymers are designated as statistical copolymers). The term “arranged in a periodic manner” means that the repeating units occur in the copolymer in a repeated pattern (e.g., a periodic copolymer consisting of repeating units A and B may have the formula (-A-B-A-A-A-B—B—B—)ₙ). The term “arranged in an alternating manner” means that the corresponding copolymer has regular alternating repeating units, such as in the formula: -A-B-A-B-A-B-A-B-A-B—, or -(-A-B—)ₙ—. The term “arranged in a block wise manner” means that the corresponding copolymer comprises at least two homopolymer subunits (i.e., blocks) linked by covalent bond. Block copolymers can have two (a diblock copolymer), three (a triblock copolymer), or more numbers of distinct blocks. An example of a sequence in a triblock copolymer consisting of A and B repeating units is as follows: -(A)ₐ-(B)_{b}-(A)_{c}-, wherein a, b, and c represent the number of repeating units in the respective block.

The term “protamine” refers to any of various strongly basic proteins of relatively low molecular weight that are rich in arginine and are found associated especially with DNA in place of somatic histones in the sperm cells of various animals (as fish). In particular, the term “protamine” refers to proteins found in fish sperm that are strongly basic, are soluble in water, are not coagulated by heat, and yield chiefly arginine upon hydrolysis. In purified form, they are used in a long-acting formulation of insulin and to neutralize the anticoagulant effects of heparin. According to the disclosure, the term “protamine” as used herein is meant to comprise any protamine amino acid sequence obtained or derived from natural or biological sources including fragments thereof and multimeric forms of said amino acid sequence or fragment thereof as well as (synthesized) polypeptides which are artificial and specifically designed for specific purposes and cannot be isolated from native or biological sources.

The term “hydrophobic” as used herein with respect to a compound, group or moiety means that said compound, group or moiety is not attracted to water molecules and, when present in an aqueous solution, aggregates and excludes water molecules. In some embodiments, the term “hydrophobic” refers to any compound, group or moiety which is substantially immiscible or insoluble in aqueous solution. In some embodiments, a hydrophobic compound, group or moiety is substantially nonpolar. Examples of hydrophobic groups are hydrocarbyl groups and fluorinated (e.g., perfluorinated) hydrocarbyl groups. In some embodiments, a hydrophobic compound, group or moiety is lipophilic. In this respect, the term “lipophilic” as used herein with respect to a compound, group or moiety means that said compound, group or moiety is soluble in nonpolar solvents (such as hexane, tetrahydrofuran (THF), and/or chloroform). In some embodiments, the term “lipophilic” refers to any compound, group or moiety which is soluble in nonpolar solvents (such as hexane, tetrahydrofuran (THF), and/or chloroform) and which is substantially immiscible or insoluble in aqueous solution. Examples of lipophilic groups are hydrocarbyl groups, such as non-cyclic, preferably straight, hydrocarbyl groups (such as straight hydrocarbyl groups having at least 10 carbon atoms), e.g., the lipophilic chain of a natural lipid.

The term “perfluorinated” as used herein with respect to a compound, group or moiety means that in said compound, group or moiety all C—H moieties have been replaced with C—F moieties. For example, perfluorinated n-octanoic acid has the formula F₃C(CF₂)₆COOH.

The term “lipid” is broadly defined herein as molecules which comprise one or more hydrophobic moieties or groups (like one or more hydrophobic chains) and optionally also one or more hydrophilic moieties or groups. For example, the term “lipid” refers to a group of organic compounds that are characterized by being insoluble in water, but soluble in many organic solvents. Molecules comprising hydrophobic moieties and hydrophilic moieties are also frequently denoted as amphiphiles. Lipids are usually poorly soluble in water. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups. Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as sterol-containing metabolites such as cholesterol.

As used herein, the term “amphiphilic” refers to a molecule having both a polar portion and a non-polar portion. Often, an amphiphilic compound has a polar head attached to a long hydrophobic tail. In some embodiments, the polar portion is soluble in water, while the non-polar portion is insoluble in water. In addition, the polar portion may have either a formal positive charge, or a formal negative charge. Alternatively, the polar portion may have both a formal positive and a negative charge, and be a zwitterion or inner salt. For purposes of the disclosure, the amphiphilic compound can be, but is not limited to, one or a plurality of natural or non-natural lipids and lipid-like compounds.

The term “lipid-like material”, “lipid-like compound” or “lipid-like molecule” relates to substances that structurally and/or functionally relate to lipids but may not be considered as lipids in a strict sense. For example, the term includes compounds that are able to form amphiphilic layers as they are present in vesicles, multilamellar/unilamellar liposomes, or membranes in an aqueous environment and includes surfactants, or synthesized compounds with both hydrophilic and hydrophobic moieties. Generally speaking, the term includes molecules, which comprise hydrophilic and hydrophobic moieties with different structural organization, which may or may not be similar to that of lipids. Examples of lipid-like compounds capable of spontaneous integration into cell membranes include functional lipid constructs such as synthetic function-spacer-lipid constructs (FSL), synthetic function-spacer-sterol constructs (FSS) as well as artificial amphipathic molecules. Lipids comprising two long alkyl chains and a polar head group are generally cylindrical. The area occupied by the two alkyl chains is similar to the area occupied by the polar head group. Such lipids have low solubility as monomers and tend to aggregate into planar bilayers that are water insoluble. Traditional surfactant monomers comprising only one linear alkyl chain and a hydrophilic head group are generally cone shaped. The hydrophilic head group tends to occupy more molecular space than the linear alkyl chain. In some embodiments, surfactants tend to aggregate into spherical or elliptoid micelles that are water soluble. While lipids also have the same general structure as surfactants—a polar hydrophilic head group and a nonpolar hydrophobic tail-lipids differ from surfactants in the shape of the monomers, in the type of aggregates formed in solution, and in the concentration range required for aggregation. As used herein, the term “lipid” is to be construed to cover both lipids and lipid-like materials unless otherwise indicated herein or clearly contradicted by context.

Specific examples of amphiphilic compounds that may be included in an amphiphilic layer include, but are not limited to, phospholipids, aminolipids and sphingolipids. In certain embodiments, the amphiphilic compound is a lipid.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphiphilic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH. An example of a neutral zwitterionic lipid is a phospholipid.

The term “functional moiety” as used herein relates to a group of atoms in a molecule with distinctive chemical properties, wherein the atoms of the functional moiety are linked to each other and to the rest of the molecule by covalent bonds. Preferably, the atoms of the functional moiety comprise at least one atom selected from the group consisting of O, N, and S. Functional moieties may be monovalent (such as hydroxy, cyano, nitro, or amide (e.g., —C(O)NHCH₃)) or divalent (such as amide (e.g., —C(O)NH—), carbonyl (—C(O)—), or ester (e.g., —OC(O)—). In some embodiments, the functional moiety provides hydrophilicity to the group to which the functional moiety is bound, e.g., by providing at least one hydrogen bond acceptor/donor and/or at least one charge (positive or negative) to the group to which the functional moiety is bound. In certain embodiments, the functional moiety comprises a hydrogen bond acceptor (such as a carbonyl moiety), a hydrogen bond donor (such as a hydroxyl moiety, —NH— (of, e.g., an amide moiety), or thiol moiety) or both (e.g., an amide moiety), and/or is charged (e.g., phosphate, amino, or ammonium moiety). Examples of monovalent functional moieties include hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino (imine), imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties. Examples of divalent functional moieties include ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino (imine), imidothioate, thionylamido, carbonate, carbonodithioate, carbonotrithioate, guanidino (imidamido), carbamimidate, carbonothioate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties.

The term “hydroxyl” or “hydroxy” as used herein with respect to a functional moiety, in particular as component of a linker, relates to the group —OH.

The term “halogen” as used herein with respect to a functional moiety, in particular as component of a linker, means fluoro, choloro, bromo, or iodo.

The term “cyano” as used herein with respect to a functional moiety, in particular as component of a linker, relates to the group —CN.

The term “azido” as used herein with respect to a functional moiety, in particular as component of a linker, relates to the group N₃.

The term “nitro” as used herein with respect to a functional moiety, in particular as component of a linker, relates to the group —NO₂.

The term “amino” as used herein with respect to a functional moiety, in particular as component of a linker, includes unsubstituted amino (i.e., the group —NH₂) and substituted amino (i.e., mono- or disubstituted amino, wherein one or two of the hydrogen atoms have been replaced with a group other than hydrogen). An amino group may be monovalent (e.g., —NRR, wherein each R is independently H or an organic group, such as R⁷² or R⁷³ as defined below) or divalent (e.g., —NR—, wherein R is H or an organic group, such as R⁷² as defined below). In some embodiments, the term “amino” means the group —N(R⁷²)(R⁷³), wherein R⁷² and R⁷³ are, in each case, independently selected from the group consisting of —H, alkyl, alkenyl, alkynyl, cycloalkyl, aryl, heteroaryl, and heterocyclyl, or R⁷² and R⁷³ may join together with the nitrogen atom to which they are attached to form the group —N═CR⁷⁵R⁷⁶, wherein each of the alkyl, alkenyl, alkynyl, cycloalkyl, aryl, heteroaryl, and heterocyclyl groups is optionally substituted with one or more (such as 1 to the maximum number of hydrogen atoms bound to the alkyl, alkenyl, alkynyl, cycloalkyl, aryl, heteroaryl, or heterocyclyl group, e.g., 1, 2, 3, 4, 5, 6, 7, 8, 9, or up to 10, such as between 1 to 5, 1 to 4, or 1 to 3, or 1 or 2) independently selected R⁷⁰; R⁷⁵ and R⁷⁶ are independently selected from the group consisting of —H, alkyl, alkenyl, alkynyl, cycloalkyl, aryl, heteroaryl, heterocyclyl, and —NH_{y}R⁸⁰_{2-y}, or R⁷⁵ and R⁷⁶ may join together with the atom to which they are attached to form a ring which is optionally substituted with one or more (such as 1 to the maximum number of hydrogen atoms bound to the ring, e.g., 1, 2, 3, 4, 5, 6, 7, 8, 9, or up to 10, such as between 1 to 5, 1 to 4, or 1 to 3, or 1 or 2) independently selected R⁷⁰, wherein each of the alkyl, alkenyl, alkynyl, cycloalkyl, aryl, heteroaryl, and heterocyclyl groups is optionally substituted with one or more (such as 1 to the maximum number of hydrogen atoms bound to the alkyl, alkenyl, alkynyl, cycloalkyl, aryl, heteroaryl, or heterocyclyl group, e.g., 1, 2, 3, 4, 5, 6, 7, 8, 9, or up to 10, such as between 1 to 5, 1 to 4, or 1 to 3, or 1 or 2) independently selected R⁷⁰; y is an integer from 0 to 2; R⁸⁰ is selected from the group consisting of alkyl, alkenyl, alkynyl, cycloalkyl, aryl, heteroaryl, and heterocyclyl, wherein each of the alkyl, alkenyl, alkynyl, cycloalkyl, aryl, heteroaryl, and heterocyclyl groups is optionally substituted with one or more (such as 1 to the maximum number of hydrogen atoms bound to the alkyl, alkenyl, alkynyl, cycloalkyl, aryl, heteroaryl, or heterocyclyl group, e.g., 1, 2, 3, 4, 5, 6, 7, 8, 9, or up to 10, such as between 1 to 5, 1 to 4, or 1 to 3, or 1 or 2) independently selected R⁷⁰; and R⁷⁰ is other than H, preferably a 1^{st} level substituent, a 2^{nd} level substituent, or a 3^{rd} level substituent as disclosed herein. In some embodiments, each of R⁷² and R⁷³ is independently H or a hydrocarbyl group, such as selected from the group consisting of H, C₁₋₆ alkyl, aryl, and aryl(C₁₋₆ alkyl), wherein each of the hydrocarbyl groups (such as each of the C₁₋₆ alkyl, aryl, and aryl(C₁₋₆ alkyl) groups) is optionally substituted with one or more (such as 1 to the maximum number of hydrogen atoms bound to the hydrocarbyl group (such as C₁₋₆ alkyl, aryl, or aryl(C₁₋₆ alkyl) group)), e.g., 1, 2, 3, 4, 5, 6, 7, 8, 9, or up to 10, such as between 1 to 5, 1 to 4, or 1 to 3, or 1 or 2) independently selected R⁷⁰.

The term “ammonium” as used herein with respect to a functional moiety, in particular as component of a linker, relates to the group —N⁺ (R⁷²)₂(R⁷³), wherein R⁷² and R⁷³ are as defined for the term “amino”.

The term “thiol” or “sulfanyl” as used herein with respect to a functional moiety, in particular as component of a linker, relates to the group —SH.

The term “disulfanyl” as used herein with respect to a functional moiety, in particular as component of a linker, relates to the group —SSH.

The term “carboxyl” or “carboxy” as used herein with respect to a functional moiety, in particular as component of a linker, relates to the group —COOH.

The term “amide” or “amido” as used herein with respect to a functional moiety, in particular as component of a linker, relates to a group comprising the structure —C(O)NH— (including its isomerically arranged structure —NHC(O)—, unless it is specified to the contrary). Preferably, each of both ends of the amide structure is covalently linked to a C atom of the same organic group or of two separate organic groups (e.g., an alkylene group as further component of the linker) (if both ends are linked to the same organic group the amide moiety is also referred to as lactam). An amide group may be monovalent (e.g., —C(O)NRR or —NRC(O)R, wherein each R is independently H or an organic group, such as one of the organic groups specified in the definition of R⁷² indicated above in the definition of the term “amino”) or divalent (e.g., —C(O)NR— or —NRC(O)—, wherein R is H or an organic group, such as one of the organic groups specified in the definition of R⁷² indicated above in the definition of the term “amino”).

The term “ester” as used herein with respect to a functional moiety, in particular as component of a linker, relates to a group comprising the structure —C(O)O— (including its isomerically arranged structure —OC(O)—, unless it is specified to the contrary). Preferably, each of both ends of the ester structure is covalently linked to a C atom of the same organic group or of two separate organic groups (e.g., an alkylene group as further component of the linker) (if both ends are linked to the same organic group the ester moiety is also referred to as lactone). An ester group may be monovalent (e.g., —C(O)OR or —OC(O)R, wherein R is independently an organic group, such as one of the organic groups specified in the definition of R⁷² indicated above in the definition of the term “amino”) or divalent (e.g., —C(O)O— or —OC(O)—)

The term “ether” as used herein with respect to a functional moiety, in particular as component of a linker, relates to a group comprising the structure —O—, wherein each of both ends of the ether structure is covalently linked to a C atom of the same organic group or of two separate organic groups (e.g., an alkylene group as further component of the linker). An ether group may be monovalent (e.g., —OR, wherein R is an organic group, such as one of the organic groups specified in the definition of R⁷² indicated above in the definition of the term “amino”) or divalent (e.g., —O—).

The term “sulfide” or “thioether” as used herein with respect to a functional moiety, in particular as component of a linker, relates to a group comprising the structure —S—, wherein each of both ends of the sulfide structure is covalently linked to a C atom of the same organic group or of two separate organic groups (e.g., an alkylene group as further component of the linker). A sulfide group may be monovalent (e.g., —SR, wherein R is an organic group, such as one of the organic groups specified in the definition of R⁷² indicated above in the definition of the term “amino”) or divalent (e.g., —S—).

The term “disulfide” as used herein with respect to a functional moiety, in particular as component of a linker, relates to a group comprising the structure —SS—, wherein each of both ends of the disulfide structure is covalently linked to a C atom of the same organic group or of two separate organic groups (e.g., an alkylene group as further component of the linker). A disulfide group may be monovalent (e.g., —SSR, wherein R is an organic group, such as one of the organic groups specified in the definition of R⁷² indicated above in the definition of the term “amino”) or divalent (e.g., —SS—).

The term “diselenide” as used herein with respect to a functional moiety, in particular as component of a linker, relates to a group comprising the structure —SeSe—, wherein each of both ends of the diselenide structure is covalently linked to a C atom of the same organic group or of two separate organic groups (e.g., an alkylene group as further component of the linker). A diselenide group may be monovalent (e.g., —SeSeR, wherein R is an organic group, such as one of the organic groups specified in the definition of R⁷² indicated above in the definition of the term “amino”) or divalent (e.g., —SeSe—).

The term “sulfoxide” as used herein with respect to a functional moiety, in particular as component of a linker, relates to a group comprising the sulfinyl structure —S(O)—, wherein each of both ends of the sulfoxide structure is covalently linked to a C atom of the same organic group or of two separate organic groups (e.g., an alkylene group as further component of the linker). A sulfoxide group may be monovalent (e.g., —S(O)R, wherein R is an organic group, such as one of the organic groups specified in the definition of R⁷² indicated above in the definition of the term “amino”) or divalent (as, e.g., —S(O)—).

The term “sulfone” as used herein with respect to a functional moiety, in particular as component of a linker, relates to a group comprising the sulfonyl structure —S(O)₂—, wherein each of both ends of the sulfone structure is covalently linked to a C atom of the same organic group or of two separate organic groups (e.g., an alkylene group as further component of the linker). A sulfone group may be monovalent (e.g., —S(O)₂R, wherein R is an organic group, such as one of the organic groups specified in the definition of R⁷² indicated above in the definition of the term “amino”) or divalent (as, e.g., —S(O)₂—).

The term “sulfite” as used herein with respect to a functional moiety, in particular as component of a linker, relates to a group comprising the structure —OS(O)O—, wherein one of the two ends of the sulfite structure is covalently linked to a C atom of an organic group and the other end is covalently linked to H or to a C atom of the same or another organic group (e.g., an alkylene group as further component of the linker). A sulfite group may be monovalent (e.g., —OS(O)OR, wherein R is H or an organic group, such as one of the organic groups specified in the definition of R⁷² indicated above in the definition of the term “amino”) or divalent (e.g., —OS(O)O—).

The term “sulfate” as used herein with respect to a functional moiety, in particular as component of a linker, relates to a group comprising the structure —OS(O)₂O—, wherein one of the two ends of the sulfate structure is covalently linked to a C atom of an organic group and the other end is covalently linked to H or to a C atom of the same or another organic group (e.g., an alkylene group as further component of the linker). A sulfate group may be monovalent (e.g., —OS(O)₂OR, wherein R is H or an organic group, such as one of the organic groups specified in the definition of R⁷² indicated above in the definition of the term “amino”) or divalent (e.g., —OS(O)₂O—).

The term “phosphate” as used herein with respect to a functional moiety, in particular as component of a linker, relates to a group comprising the structure —OP(O)(OR)O—, wherein one of the two ends of the phosphate structure is covalently linked to a C atom of an organic group (e.g., an alkylene group as further component of the linker) and the other end is covalently linked to H or to a C atom of the same or another organic group (R is H or an organic group, such as one of the organic groups specified in the definition of R⁷² indicated above in the definition of the term “amino”). A phosphate group may be monovalent (e.g., —OP(O)(OR)₂, wherein each R is independently H or an organic group, such as one of the organic groups specified in the definition of R⁷² indicated above in the definition of the term “amino”) or divalent (e.g., —OP(O)(OR)O—, wherein R is independently H or an organic group, such as one of the organic groups specified in the definition of R⁷² indicated above in the definition of the term “amino”).

The term “sulfinamide” as used herein with respect to a functional moiety, in particular as component of a linker, relates to a group comprising the structure —S(O)N(R)—, wherein the S end of the sulfinamide structure is covalently linked to a C atom of an organic group (e.g., an alkylene group as further component of the linker) and the N end is covalently linked to H or to a C atom of the same or another organic group (R is H or an organic group, such as one of the organic groups specified in the definition of R⁷² indicated above in the definition of the term “amino”). A sulfinamide group may be monovalent (e.g., —S(O)N(R)₂, wherein each R is independently H or an organic group, such as one of the organic groups specified in the definition of R⁷² indicated above in the definition of the term “amino”) or divalent (e.g., —S(O)N(R)—, wherein R is H or an organic group, such as one of the organic groups specified in the definition of R⁷² indicated above in the definition of the term “amino”).

The term “sulfonamide” as used herein with respect to a functional moiety, in particular as component of a linker, relates to a group comprising the structure —S(O)₂N(R)—, wherein the S end of the sulfonamide structure is covalently linked to a C atom of an organic group (e.g., an alkylene group as further component of the linker) and the N end is covalently linked to H or to a C atom of the same or another organic group (R is H or an organic group, such as one of the organic groups specified in the definition of R⁷² indicated above in the definition of the term “amino”). A sulfonamide group may be monovalent (e.g., —S(O)₂N(R)₂, wherein each R is independently H or an organic group, such as one of the organic groups specified in the definition of R⁷² indicated above in the definition of the term “amino”) or divalent (e.g., —S(O)₂N(R)—, wherein R is H or an organic group, such as one of the organic groups specified in the definition of R⁷² indicated above in the definition of the term “amino”).

The term “sulfamate” as used herein with respect to a functional moiety, in particular as component of a linker, relates to a group comprising the structure —OS(O)₂N(R)— (including its isomerically arranged structure —N(R) S(O)₂O—, unless it is specified to the contrary), wherein one of both ends of the sulfamate structure is covalently linked to a C atom of an organic group (e.g., an alkylene group as further component of the linker) and the other end is covalently linked to H or to a C atom of the same or another organic group (R is H or an organic group, such as one of the organic groups specified in the definition of R⁷² indicated above in the definition of the term “amino”). A sulfamate group may be monovalent (e.g., —OS(O)₂N(R)₂ or —N(R) S(O)₂OR, wherein each R is independently H or an organic group, such as one of the organic groups specified in the definition of R⁷² indicated above in the definition of the term “amino”) or divalent (e.g., —OS(O)₂N(R)— or —N(R)S(O)₂O—, wherein each R is independently H or an organic group, such as one of the organic groups specified in the definition of R⁷² indicated above in the definition of the term “amino”).

The term “sulfurous diamide” as used herein with respect to a functional moiety, in particular as component of a linker, relates to a group comprising the structure —N(R)S(O)N(R)—, wherein one of both ends of the sulfurous diamide structure is covalently linked to a C atom of an organic group (e.g., an alkylene group as further component of the linker) and the other end is covalently linked to H or to a C atom of the same or another organic group (each R is independently H or an organic group, such as one of the organic groups specified in the definition of R⁷² indicated above in the definition of the term “amino”). A sulfurous diamide group may be monovalent (e.g., —N(R)S(O)N(R)₂, wherein each R is independently H or an organic group, such as one of the organic groups specified in the definition of R⁷² indicated above in the definition of the term “amino”) or divalent (e.g., —N(R)S(O)N(R)—, wherein each R is independently H or an organic group, such as one of the organic groups specified in the definition of R⁷² indicated above in the definition of the term “amino”).

The term “sulfuric diamide” as used herein with respect to a functional moiety, in particular as component of a linker, relates to a group comprising the structure —N(R) S(O)₂N(R)—, wherein one of both ends of the sulfuric diamide structure is covalently linked to a C atom of an organic group (e.g., an alkylene group as further component of the linker) and the other end is covalently linked to H or to a C atom of the same or another organic group (each R is independently H or an organic group, such as one of the organic groups specified in the definition of R⁷² indicated above in the definition of the term “amino”). A sulfuric diamide group may be monovalent (e.g., —N(R) S(O)₂N(R)₂, wherein each R is independently H or an organic group, such as one of the organic groups specified in the definition of R⁷² indicated above in the definition of the term “amino”) or divalent (e.g., —N(R) S(O)₂N(R)—, wherein each R is independently H or an organic group, such as one of the organic groups specified in the definition of R⁷² indicated above in the definition of the term “amino”).

The term “urea” as used herein with respect to a functional moiety, in particular as component of a linker, relates to a group comprising the structure —N(R)C(O)N(R)—, wherein one of both ends of the urea structure is covalently linked to a C atom of an organic group (e.g., an alkylene group as further component of the linker) and the other end is covalently linked to H or to a C atom of the same or another organic group (each R is independently H or an organic group, such as one of the organic groups specified in the definition of R⁷² indicated above in the definition of the term “amino”). An urea group may be monovalent (e.g., —N(R)C(O)N(R)₂, wherein each R is independently H or an organic group, such as one of the organic groups specified in the definition of R⁷² indicated above in the definition of the term “amino”) or divalent (e.g., —N(R)C(O)N(R)—, wherein each R is H or an organic group, such as one of the organic groups specified in the definition of R⁷² indicated above in the definition of the term “amino”).

The term “thiourea” as used herein with respect to a functional moiety, in particular as component of a linker, relates to a group comprising the structure —N(R)C(S)N(R)—, wherein one of both ends of the thiourea structure is covalently linked to a C atom of an organic group (e.g., an alkylene group as further component of the linker) and the other end is covalently linked to H or to a C atom of the same or another organic group (each R is independently H or an organic group, such as one of the organic groups specified in the definition of R⁷² indicated above in the definition of the term “amino”). A thiourea group may be monovalent (e.g., —N(R)C(S)N(R)₂, wherein each R is independently H or an organic group, such as one of the organic groups specified in the definition of R⁷² indicated above in the definition of the term “amino”) or divalent (e.g., —N(R)C(S)N(R)—, wherein each R is H or an organic group, such as one of the organic groups specified in the definition of R⁷² indicated above in the definition of the term “amino”).

The term “carbonyl” as used herein with respect to a functional moiety, in particular as component of a linker, relates to a group comprising the structure —C(O)—, wherein one of both ends of the carbonyl structure is covalently linked to a C atom of an organic group (e.g., an alkylene group as further component of the linker) and the other end is covalently linked to H or to a C atom of the same or another organic group (if both ends are linked to C atoms of organic groups the carbonyl moiety is also referred to as “keto” moiety). A carbonyl group may be monovalent (e.g., —C(O)R, wherein R is H or an organic group, such as one of the organic groups specified in the definition of R⁷² indicated above in the definition of the term “amino”) or divalent (e.g., —C(O)—).

The term “thiocarbonyl” as used herein with respect to a functional moiety, in particular as component of a linker, relates to a group comprising the structure —C(S)—, wherein one of both ends of the thiocarbonyl structure is covalently linked to a C atom of an organic group (e.g., an alkylene group as further component of the linker) and the other end is covalently linked to H or to a C atom of the same or another organic group. A thiocarbonyl group may be monovalent (e.g., —C(S)R, wherein R is H or an organic group, such as one of the organic groups specified in the definition of R⁷² indicated above in the definition of the term “amino”) or divalent (e.g., —C(S)—).

The term “orthoester” as used herein with respect to a functional moiety, in particular as component of a linker, relates to a moiety comprising a C atom to which three alkoxy groups (i.e., —OR, wherein R is an organic group (e.g., an alkylene group as further component of the linker), such one of the organic groups specified in the definition of R⁷² indicated above in the definition of the term “amino”) are attached. An exemplary formula of an orthoester comprises the structure (—O)ᵣC(OR)₃₋ᵣ—, wherein each R is independently an organic group, such as independently selected from the organic groups specified in the definition of R⁷² indicated above in the definition of the term “amino”; r is 1 or 2; and each of both ends of the orthoester structure is covalently linked to a C atom of a further organic group or of two further separate organic groups. In some embodiments, an orthoester comprises the structure (—O)ᵣC(OR²⁵)₃₋ᵣ—, wherein each R²⁵ is independently a hydrocarbyl group, such as C₁₋₆ alkyl, aryl, and aryl(C₁₋₆ alkyl) which is optionally substituted (e.g., with one or more 1^{st} level substituents, 2^{nd} level substituents, or 3^{rd} level substituents as defined herein); r is 1 or 2; and each of both ends of the orthoester structure is covalently linked to a C atom of a further organic group or of two further separate organic groups. An orthoester group may be monovalent (e.g., —C(OR)₃ or —OC(OR)₂R, wherein each R is an organic group, such as independently selected from the organic groups specified in the definition of R⁷² indicated above in the definition of the term “amino”) or divalent (e.g., (—O)₂C(OR)(R) or —OC(OR)₂—, wherein each R is an organic group, such as independently selected from the organic groups specified in the definition of R⁷² indicated above in the definition of the term “amino”).

The term “thioate” or “thioester” as used herein with respect to a functional moiety, in particular as component of a linker, relates to a group comprising the structure —SC(O)— (including its isomerically arranged structure —C(O)S—, unless it is specified to the contrary), wherein each of both ends of the thioate structure is covalently linked to a C atom of the same organic group or of two separate organic groups (e.g., an alkylene group as further component of the linker). A thioate group may be monovalent (e.g., —SC(O)R or —C(O)SR, wherein each R is independently H or an organic group, such as one of the organic groups specified in the definition of R⁷² indicated above in the definition of the term “amino”) or divalent (e.g., —SC(O)— or —C(O)S—).

The term “dithioate” as used herein with respect to a functional moiety, in particular as component of a linker, relates to a group comprising the structure —SC(S)— (including its isomerically arranged structure —C(S)S—, unless it is specified to the contrary), wherein each of both ends of the dithioate structure is covalently linked to a C atom of the same organic group or of two separate organic groups (e.g., an alkylene group as further component of the linker). A dithioate group may be monovalent (e.g., —SC(S) R or —C(S) SR, wherein each R is independently H or an organic group, such as one of the organic groups specified in the definition of R⁷² indicated above in the definition of the term “amino”) or divalent (e.g., —SC(S)— or —C(S)S—).

The term “imidate” as used herein with respect to a functional moiety, in particular as component of a linker, relates to a group comprising the structure —OC(═NR)— (including its isomerically arranged structure —C(═NR)O—, unless it is specified to the contrary), wherein each of both ends of the imidate structure is covalently linked to a C atom of the same organic group or of two separate organic groups (e.g., an alkylene group as further component of the linker) (each R is independently H or an organic group, such as one of the organic groups specified in the definition of R⁷² indicated above in the definition of the term “amino”). An imidate group may be monovalent (e.g., —OC(═NR) R′ or —C(═NR) OR′, wherein each R is independently H or an organic group, such as one of the organic groups specified in the definition of R⁷² indicated above in the definition of the term “amino”; and each R′ is independently an organic group, such as one of the organic groups specified in the definition of R⁷² indicated above in the definition of the term “amino”) or divalent (e.g., —OC(═NR)— or —C(═NR)O—, wherein each R is independently H or an organic group, such as one of the organic groups specified in the definition of R⁷² indicated above in the definition of the term “amino”).

The term “imino” or “imine” as used herein with respect to a functional moiety, in particular as component of a linker, relates to a group comprising the structure —C(═NR)—, wherein one of both ends of the imino structure is covalently linked to a C atom of an organic group (e.g., an alkylene group as further component of the linker) and the other end is covalently linked to H or to a C atom of the same or another organic group (each R is independently H or an organic group, such as one of the organic groups specified in the definition of R⁷² indicated above in the definition of the term “amino”). The moiety-C(═NR) H is also called “aldimine”, and the moiety-C(═NR) R′, wherein R′ is an organic group, such as one of the organic groups specified in the definition of R⁷² indicated above in the definition of the term “amino”), is also called “ketimine”. An imino group may be monovalent (e.g., —C(═NR) R, wherein each R is independently H or an organic group, such as one of the organic groups specified in the definition of R⁷² indicated above in the definition of the term “amino”; and each R′ is independently an organic group, such as one of the organic groups specified in the definition of R⁷² indicated above in the definition of the term “amino”) or divalent (e.g., —C(═NR)—, wherein R is H or an organic group, such as one of the organic groups specified in the definition of R⁷² indicated above in the definition of the term “amino”).

The term “imidothioate” as used herein with respect to a functional moiety, in particular as component of a linker, relates to a group comprising the structure —C(═NR)S— (including its isomerically arranged structure —SC(═NR)—, unless it is specified to the contrary), wherein one of both ends of the imidothioate structure is covalently linked to a C atom of an organic group (e.g., an alkylene group as further component of the linker) and the other end is covalently linked to H or to a C atom of the same or another organic group (each R is independently H or an organic group, such as one of the organic groups specified in the definition of R⁷² indicated above in the definition of the term “amino”). An imidothioate group may be monovalent (e.g., —C(═NR) SR or —SC(═NR)R, wherein each R is independently H or an organic group, such as one of the organic groups specified in the definition of R⁷² indicated above in the definition of the term “amino”) or divalent (e.g., —C(═NR)S— or —SC(═NR)—, wherein each R is independently H or an organic group, such as one of the organic groups specified in the definition of R⁷² indicated above in the definition of the term “amino”).

The term “thionylamino” as used herein with respect to a functional moiety, in particular as component of a linker, relates to a group comprising the structure —C(S)NR— (including its isomerically arranged structure —N(R)C(S)—, unless it is specified to the contrary), wherein one of both ends of the thionylamino structure is covalently linked to a C atom of an organic group (e.g., an alkylene group as further component of the linker) and the other end is covalently linked to H or to a C atom of the same or another organic group (each R is independently H or an organic group, such as one of the organic groups specified in the definition of R⁷² indicated above in the definition of the term “amino”). A thionylamino group may be monovalent (e.g., —C(S)NRR or —N(R)C(S)R, wherein each R is independently H or an organic group, such as one of the organic groups specified in the definition of R⁷² indicated above in the definition of the term “amino”) or divalent (e.g., —C(S)NR— or —N(R)C(S)—, wherein each R is independently H or an organic group, such as one of the organic groups specified in the definition of R⁷² indicated above in the definition of the term “amino”).

The term “carbonate” as used herein with respect to a functional moiety, in particular as component of a linker, relates to a group comprising the structure —OC(O)O—, wherein each of both ends of the carbonate structure is covalently linked to a C atom of an organic group (e.g., an alkylene group as further component of the linker). A carbonate group may be monovalent (e.g., —OC(O)OR′, wherein R′ is an organic group, such as one of the organic groups specified in the definition of R⁷² indicated above in the definition of the term “amino”) or divalent (e.g., —OC(O)O—).

The term “carbonothioate” as used herein with respect to a functional moiety, in particular as component of a linker, relates to a group comprising the structure —OC(S)O— or —OC(O)S— (including its isomerically arranged structure —SC(O)O—, unless it is specified to the contrary), wherein each of both ends of the carbonothioate structure is covalently linked to a C atom of an organic group (e.g., an alkylene group as further component of the linker). A carbonothioate group may be monovalent (e.g., —OC(S) OR′ or —OC(O)SR′ or —SC(O)OR′, wherein each R′ is independently an organic group, such as one of the organic groups specified in the definition of R⁷² indicated above in the definition of the term “amino”) or divalent (e.g., —OC(S)O— or —OC(O)S— or —SC(O)O—).

The term “carbonodithioate” as used herein with respect to a functional moiety, in particular as component of a linker, relates to a group comprising the structure —SC(O)S— or —OC(S)S— (including its isomerically arranged structure —SC(S)O—, unless it is specified to the contrary), wherein each of both ends of the carbonodithioate structure is covalently linked to a C atom of an organic group (e.g., an alkylene group as further component of the linker). A carbonodithioate group may be monovalent (e.g., —SC(O)SR′—OC(S) SR′ or —SC(S) OR′, wherein each R′ is independently an organic group, such as one of the organic groups specified in the definition of R⁷² indicated above in the definition of the term “amino”) or divalent (e.g., —SC(O)S— or —OC(S)S— or —SC(S)O—).

The term “carbonotrithioate” as used herein with respect to a functional moiety, in particular as component of a linker, relates to a group comprising the structure —SC(S)S—, wherein each of both ends of the carbonotrithioate structure is covalently linked to a C atom of an organic group (e.g., an alkylene group as further component of the linker). A carbonotrithioate group may be monovalent (e.g., —SC(S) SR, wherein R is an organic group, such as one of the organic groups specified in the definition of R⁷² indicated above in the definition of the term “amino”) or divalent (e.g., —SC(S)S—).

The term “guanidino” or “imidamido” as used herein with respect to a functional moiety, in particular as component of a linker, relates to a group comprising the structure —N(R)C(═NR)NR— (wherein each R is independently H or an organic group, such as one of the organic groups specified in the definition of R⁷² indicated above in the definition of the term “amino”), wherein one of both ends of the guanidino structure is covalently linked to a C atom of an organic group (e.g., an alkylene group as further component of the linker) and the other end is covalently linked to H or to a C atom of the same or another organic group. A guanidino group may be monovalent (e.g., —N(R)C(═NR)NRR, wherein each R is independently H or an organic group, such as one of the organic groups specified in the definition of R⁷² indicated above in the definition of the term “amino”) or divalent (e.g., —N(R)C(═NR)NR—, wherein each R is independently H or an organic group, such as one of the organic groups specified in the definition of R⁷² indicated above in the definition of the term “amino”).

The term “carbamimidate” as used herein with respect to a functional moiety, in particular as component of a linker, relates to a group comprising the structure —OC(═NR)NR— (including its isomerically arranged structure —N(R)C(═NR)O—, unless it is specified to the contrary), wherein the O end of the carbamimidate structure is covalently linked to a C atom of an organic group (e.g., an alkylene group as further component of the linker) and the other (N) end is covalently linked to H or to a C atom of the same or another organic group (each R is independently H or an organic group, such as one of the organic groups specified in the definition of R⁷² indicated above in the definition of the term “amino”). A carbamimidate group may be monovalent (e.g., —OC(═NR)NRR or —N(R)C(═NR) OR′, wherein each R is independently H or an organic group, such as one of the organic groups specified in the definition of R⁷² indicated above in the definition of the term “amino”; and R′ is an organic group, such as one of the organic groups specified in the definition of R⁷² indicated above in the definition of the term “amino”) or divalent (e.g., —OC(═NR)NR— or —N(R)C(═NR)O—, wherein each R is independently H or an organic group, such as one of the organic groups specified in the definition of R⁷² indicated above in the definition of the term “amino”).

The term “carbonimidate” as used herein with respect to a functional moiety, in particular as component of a linker, relates to a group comprising the structure —OC(═NR)O—, wherein each of the ends of the carbonimidate structure is covalently linked to a C atom of an organic group (e.g., an alkylene group as further component of the linker) (each R is independently H or an organic group, such as one of the organic groups specified in the definition of R⁷² indicated above in the definition of the term “amino”).

A carbonimidate group may be monovalent (e.g., —OC(═NR) OR′, wherein R is H or an organic group, such as one of the organic groups specified in the definition of R⁷² indicated above in the definition of the term “amino”; and R′ is an organic group, such as one of the organic groups specified in the definition of R⁷² indicated above in the definition of the term “amino”) or divalent (e.g., —OC(═NR)O—, wherein R is H or an organic group, such as one of the organic groups specified in the definition of R⁷² indicated above in the definition of the term “amino”).

The term “carbamate” as used herein with respect to a functional moiety, in particular as component of a linker, relates to a group comprising the structure —OC(O)NR— (including its isomerically arranged structure —N(R)C(O)O—, unless it is specified to the contrary), wherein the O end of the carbamate structure is covalently linked to a C atom of an organic group (e.g., an alkylene group as further component of the linker) and the other end (N end) is covalently linked to H or to a C atom of the same or another organic group (each R is independently H or an organic group, such as one of the organic groups specified in the definition of R⁷² indicated above in the definition of the term “amino”). A carbamate group may be monovalent (e.g., —OC(O)NRR or —N(R)C(O)OR′, wherein each R is independently H or an organic group, such as one of the organic groups specified in the definition of R⁷² indicated above in the definition of the term “amino”; and R′ is an organic group, such as one of the organic groups specified in the definition of R⁷² indicated above in the definition of the term “amino”) or divalent (e.g., —OC(O)NR— or —N(R)C(O)O—, wherein each R is independently H or an organic group, such as one of the organic groups specified in the definition of R⁷² indicated above in the definition of the term “amino”).

The term “carbamodithioate” as used herein with respect to a functional moiety, in particular as component of a linker, relates to a group comprising the structure —SC(S)NR— (including its isomerically arranged structure —N(R)C(S)S—, unless it is specified to the contrary), wherein the S end of the carbamodithioate structure is covalently linked to a C atom of an organic group (e.g., an alkylene group as further component of the linker) and the other end (N end) is covalently linked to H or to a C atom of the same or another organic group (each R is independently H or an organic group, such as one of the organic groups specified in the definition of R⁷² indicated above in the definition of the term “amino”). A carbamodithioate group may be monovalent (e.g., —SC(S)NRR or —N(R)C(S) SR′, wherein each R is independently H or an organic group, such as one of the organic groups specified in the definition of R⁷² indicated above in the definition of the term “amino”; and R′ is an organic group, such as one of the organic groups specified in the definition of R⁷² indicated above in the definition of the term “amino”) or divalent (e.g., —SC(S)NR— or —N(R)C(S)S—, wherein each R is independently H or an organic group, such as one of the organic groups specified in the definition of R⁷² indicated above in the definition of the term “amino”).

The term “carbonodithioimidate” as used herein with respect to a functional moiety, in particular as component of a linker, relates to a group comprising the structure —SC(═NR)S—, wherein each of the ends of the carbonodithioimidate structure is covalently linked to a C atom of an organic group (e.g., an alkylene group as further component of the linker) (each R is independently H or an organic group, such as one of the organic groups specified in the definition of R⁷² indicated above in the definition of the term “amino”). A carbonodithioimidate group may be monovalent (e.g., —SC(═NR) SR′, wherein R is H or an organic group, such as one of the organic groups specified in the definition of R⁷² indicated above in the definition of the term “amino”; and R′ is an organic group, such as one of the organic groups specified in the definition of R⁷² indicated above in the definition of the term “amino”) or divalent (e.g., —SC(═NR)S—, wherein R is H or an organic group, such as one of the organic groups specified in the definition of R⁷² indicated above in the definition of the term “amino”).

The term “carbamimidothioate” as used herein with respect to a functional moiety, in particular as component of a linker, relates to a group comprising the structure —SC(═NR)NR— (including its isomerically arranged structure —N(R)C(═NR)S—, unless it is specified to the contrary), wherein the S end of the carbamimidothioate structure is covalently linked to a C atom of an organic group (e.g., an alkylene group as further component of the linker) and the other end (N end) is covalently linked to H or to a C atom of the same or another organic group (each R is independently H or an organic group, such as one of the organic groups specified in the definition of R⁷² indicated above in the definition of the term “amino”). A carbamimidothioate group may be monovalent (e.g., —SC(═NR)NRR or —N(R)C(═NR) SR′, wherein each R is independently H or an organic group, such as one of the organic groups specified in the definition of R⁷² indicated above in the definition of the term “amino”; and R′ is an organic group, such as one of the organic groups specified in the definition of R⁷² indicated above in the definition of the term “amino”) or divalent (e.g., —SC(═NR)NR— or —N(R)C(═NR)S—, wherein each R is independently H or an organic group, such one of the organic groups specified in the definition of R⁷² indicated above in the definition of the term “amino”).

The term “carbamothioate” as used herein with respect to a functional moiety, in particular as component of a linker, relates to a group comprising the structure —N(R)C(O)S— or —N(R)C(S)O— (including their isomerically arranged structures-SC(O)NR— or —OC(S)NR—, unless it is specified to the contrary), wherein the O/S end of the carbamothioate structure is covalently linked to a C atom of an organic group (e.g., an alkylene group as further component of the linker) and the other end (N end) is covalently linked to a C atom of the same or another organic group (each R is independently H or an organic group, such as one of the organic groups specified in the definition of R⁷² indicated above in the definition of the term “amino”). A carbamothioate group may be monovalent (e.g., —N(R)C(O)SR′ or —N(R)C(S) OR′ or —SC(O)NRR or —OC(S)NRR, wherein each R is independently H or an organic group, such as one of the organic groups specified in the definition of R⁷² indicated above in the definition of the term “amino”; and each R′ is independently an organic group, such as one of the organic groups specified in the definition of R⁷² indicated above in the definition of the term “amino”) or divalent (e.g., —N(R)C(O)S— or —N(R)C(S)O— or —SC(O)NR— or —OC(S)NR—, wherein each R is independently H or an organic group, such as one of the organic groups specified in the definition of R⁷² indicated above in the definition of the term “amino”).

The term “carbonimidothioate” as used herein with respect to a functional moiety, in particular as component of a linker, relates to a group comprising the structure —OC(═NR)S— (including its isomerically arranged structure —SC(═NR)O—, unless it is specified to the contrary), wherein the O/S end of the carbonimidothioate structure is covalently linked to a C atom of an organic group (e.g., an alkylene group as further component of the linker) and the other end (N end) is covalently linked to H or to a C atom of the same or another organic group (each R is independently H or an organic group, such as one of the organic groups specified in the definition of R⁷² indicated above in the definition of the term “amino”). A carbonimidothioate group may be monovalent (e.g., —OC(═NR) SR′ or —SC(═NR) OR′, wherein each R is independently H or an organic group, such as one of the organic groups specified in the definition of R⁷² indicated above in the definition of the term “amino”; and each R′ is independently an organic group, such as one of the organic groups specified in the definition of R⁷² indicated above in the definition of the term “amino”) or divalent (e.g., —OC(═NR)S— or —SC(═NR)O—, wherein each R is independently H or an organic group, such as one of the organic groups specified in the definition of R⁷² indicated above in the definition of the term “amino”).

The term “acylhydrazone” as used herein with respect to a functional moiety, in particular as component of a linker, relates to a group comprising the structure —C(R′) (═N—N(R)C(O)—) (including its isomerically arranged structure (—C(O)(N(R)—N═) C(R′)—, unless it is specified to the contrary) and/or ═C(═N—N(R)C(O)R′), wherein each R′ is independently an organic group, such as one of the organic groups specified in the definition of R⁷² indicated above in the definition of the term “amino”; each R is H or an organic group, such as such as one of the organic groups specified in the definition of R⁷² indicated above in the definition of the term “amino”; and each of both ends of the acylhydrazone structure is covalently linked to a C atom of a further organic group or of two further separate organic groups (e.g., an alkylene group as further component of the linker). In some embodiments, an acylhydrazone comprises the structure —C(R²⁵)(═N—N(R²⁶)C(O)—) (including its isomerically arranged structure (—C(O)(N(R²⁶)—N═)C(R²⁵)—, unless it is specified to the contrary) and/or ═C(═N—N(R²⁶)C(O)R²⁵), wherein each R²⁵ is independently a hydrocarbyl group, such as C₁₋₆ alkyl, aryl, and aryl(C₁₋₆ alkyl) which is optionally substituted (e.g., with one or more 1^{st} level substituents, 2^{nd} level substituents, or 3^{rd} level substituents as defined herein); each R²⁶ is independently H or a hydrocarbyl group, such as C₁₋₆ alkyl, aryl, and aryl(C₁₋₆ alkyl), which is optionally substituted (e.g., with one or more 1^{st} level substituents, 2^{nd} level substituents, or 3^{rd} level substituents as defined herein); and each of both ends of the acylhydrazone structure is covalently linked to a C atom of a further organic group or of two further separate organic groups. Exemplary chemical structures of an acylhydrazone are shown below:

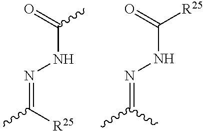

wherein each [symbol] represents the bond by which the acylhydrazone is covalently linked to the further organic group(s) (e.g., an alkylene group as further component of the linker). A acylhydrazone group may be monovalent (e.g., —C(R′) (═N—N(R)C(O)R′) or —C(O)(N(R)—N═) C(R′)₂, wherein each R is independently H or an organic group, such as one of the organic groups specified in the definition of R⁷² indicated above in the definition of the term “amino”; and each R′ is independently an organic group, such as one of the organic groups specified in the definition of R⁷² indicated above in the definition of the term “amino”) or divalent (e.g., —C(R′) (═N—N(R)C(O)—, —C(O)(N(R)—N═) C(R′)—, or ═C(═N—N(R²⁶)C(O)R²⁵), wherein each R′ is independently an organic group, such as one of the organic groups specified in the definition of R⁷² indicated above in the definition of the term “amino”; and each R is H an organic group, such as one of the organic groups specified in the definition of R⁷² indicated above in the definition of the term “amino”.

The term “hydrazine” as used herein with respect to a functional moiety, in particular as component of a linker, relates to a group comprising the structure —N(R)N(R)—, wherein each R is independently H or an organic group, such as one of the organic groups specified in the definition of R⁷² indicated above in the definition of the term “amino”; and each of both ends of the hydrazine structure is covalently linked to a C atom of a further organic group or of two further separate organic groups (e.g., an alkylene group as further component of the linker). In some embodiments, a hydrazine comprises the structure —N(R²⁶)N(R²⁶)—, wherein each R²⁶ is independently H or a hydrocarbyl group, such as C₁₋₆ alkyl, aryl, and aryl(C₁₋₆ alkyl), which is optionally substituted (e.g., with one or more 1^{st} level substituents, 2^{nd} level substituents, or 3^{rd} level substituents as defined herein); and each of both ends of the hydrazine structure is covalently linked to a C atom of a further organic group or of two further separate organic groups. A hydrazine group may be monovalent (e.g., —N(R)N(R)₂, wherein each R is independently H or an organic group, such as one of the organic groups specified in the definition of R⁷² indicated above in the definition of the term “amino”) or divalent (e.g., —N(R)N(R)—, wherein each R′ is independently an organic group, such as one of the organic groups specified in the definition of R⁷² indicated above in the definition of the term “amino”).

The term “oxime” as used herein with respect to a functional moiety, in particular as component of a linker, relates to a group comprising the structure═C(═N(OH)), wherein each of both ends of the oxime structure is covalently linked to a C atom of the same organic group or of two separate organic groups (e.g., an alkylene group as further component of the linker). An exemplary chemical formula of an oxime is shown below:

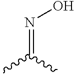 [SMILES: *=NO]

wherein each [symbol] represents the bond by which the oxime is covalently linked to the further organic group(s). An oxime group may be monovalent (e.g., —C(═N(OH))(R), wherein each R is independently H or an organic group, such as one of the organic groups specified in the definition of R⁷² indicated above in the definition of the term “amino”) or divalent (e.g., ═C(═N(OH))).

The term “acetal” as used herein with respect to a functional moiety, in particular as component of a linker, relates to a group comprising the structure —OCH(R′)O—, wherein R′ is an organic group, such as one of the organic groups specified in the definition of R⁷² indicated above in the definition of the term “amino”; and each of both O atoms of the acetal structure is covalently linked to a C atom of a further organic group or of two further separate organic groups (e.g., an alkylene group as further component of the linker). In some embodiments, an acetal comprises the structure —OCH(R²⁵)O—, wherein R²⁵ is a hydrocarbyl group, such as C₁₋₆ alkyl, aryl, and aryl(C₁₋₆ alkyl)), which is optionally substituted (e.g., with one or more 1^{st} level substituents, 2^{nd} level substituents, or 3^{rd} level substituents as defined herein); and each of both O atoms of the acetal structure is covalently linked to a C atom of a further organic group or of two further separate organic groups. An acetal group may be monovalent (e.g., —OCH(R′) OR′, wherein each R′ is independently an organic group, such as independently selected from the organic groups specified in the definition of R⁷² indicated above in the definition of the term “amino”) or divalent (e.g., —OCH(R′)O—, wherein R′ is an organic group, such as one of the organic groups specified in the definition of R⁷² indicated above in the definition of the term “amino”).

The term “hemiacetal” as used herein with respect to a functional moiety, in particular as component of a linker, relates to a group comprising the structure —OCH(OH)—, wherein each of both ends of the hemiacetal structure is covalently linked to a C atom of a further organic group or of two further separate organic groups (e.g., an alkylene group as further component of the linker). A hemiacetal group may be monovalent (e.g., —OCH(OH) OR′, wherein R′ is an organic group, such as one of the organic groups specified in the definition of R⁷² indicated above in the definition of the term “amino”) or divalent (e.g., —OCH(OH)—).

The term “ketal” as used herein with respect to a functional moiety, in particular as component of a linker, relates to a group comprising the structure —OC(R′) (R′)O—, wherein each R′ is independently an organic group, such as independently selected from the organic groups specified in the definition of R⁷² indicated above in the definition of the term “amino”; and each of both O atoms of the ketal structure is covalently linked to a C atom of a further organic group or of two further separate organic groups (e.g., an alkylene group as further component of the linker). In some embodiments, a ketal comprises the structure —OC(R²⁵)(R²⁵)O—, wherein each R²⁵ is independently a hydrocarbyl group, such as C₁₋₆ alkyl, aryl, and aryl(C₁₋₆ alkyl)), which is optionally substituted (e.g., with one or more 1^{st} level substituents, 2^{nd} level substituents, or 3^{rd} level substituents as defined herein); and each of both O atoms of the ketal structure is covalently linked to a C atom of a further organic group or of two further separate organic groups. An ketal group may be monovalent (e.g., —OC(R′) (R′) OR′, wherein each R′ is independently an organic group, such as independently selected from the organic groups specified in the definition of R⁷² indicated above in the definition of the term “amino”) or divalent (e.g., —OC(R′) (R′)O—, wherein each R′ is independently an organic group, such as one of the organic groups specified in the definition of R⁷² indicated above in the definition of the term “amino”).

The term “hemiketal” as used herein with respect to a functional moiety, in particular as component of a linker, relates to a group comprising the structure —OCR′ (OH)—, wherein R′ is an organic group, such as one of the organic groups specified in the definition of R⁷² indicated above in the definition of the term “amino”; and each of both ends of the hemiketal structure is covalently linked to a C atom of a further organic group or of two further separate organic groups (e.g., an alkylene group as further component of the linker). In some embodiments, a hemiketal comprises the structure —OCR25 (OH)—, wherein R²⁵ is a hydrocarbyl group, such as C₁₋₆ alkyl, aryl, and aryl(C₁₋₆ alkyl)), which is optionally substituted (e.g., with one or more 1^{st} level substituents, 2^{nd} level substituents, or 3^{rd} level substituents as defined herein); and each of both ends of the hemiketal structure is covalently linked to a C atom of a further organic group or of two further separate organic groups. A hemiketal group may be monovalent (e.g., —OC(R′)₂ (OH), wherein each R′ is independently an organic group, such as independently selected from the organic groups specified in the definition of R⁷² indicated above in the definition of the term “amino”) or divalent (e.g., —OCR′ (OH)—, wherein R′ is an organic group, such as one of the organic groups specified in the definition of R⁷² indicated above in the definition of the term “amino”).

The term “imide” as used herein with respect to a functional moiety, in particular within a as component of, relates to a group comprising the structure —C(O)N(R)C(O)—, wherein R is an organic group, such as one of the organic groups specified in the definition of R⁷² indicated above in the definition of the term “amino”; and each of both ends of the imide structure is covalently linked to a C atom of the same organic group or of two separate organic groups (e.g., an alkylene group as further component of the linker). An imide group may be monovalent (e.g., —C(O)N(R)C(O)R′, wherein R is independently H or an organic group, such as one of the organic groups specified in the definition of R⁷² indicated above in the definition of the term “amino”; and R′ is an organic group, such as one of the organic groups specified in the definition of R⁷² indicated above in the definition of the term “amino”) or divalent (e.g., —C(O)N(R)C(O)—, wherein R is H or an organic group, such as one of the organic groups specified in the definition of R⁷² indicated above in the definition of the term “amino”).

The term “hydrocarbyl” as used herein relates to a monovalent organic group obtained by removing one H atom from a hydrocarbon molecule. Typical examples of hydrocarbyl groups include alkyl, alkenyl, alkynyl, cycloalkyl, aryl groups, and combinations thereof (such as arylalkyl (aralkyl), etc.). Particular examples of hydrocarbyl groups are C₁₋₆ alkyl, aryl, and aryl(C₁₋₆ alkyl). In some embodiments, the hydrocarbyl group is optionally substituted (e.g., with one or more 1^{st} level substituents, 2^{nd} level substituents, or 3^{rd} level substituents as defined herein).

The term “non-cyclic” as used herein in the context of organic groups relates to open-chain organic groups which contain no rings. “Open-chain” or “acyclic” organic groups may be straight (i.e., they contain only one unbranched chain without any sidechain) or branched (i.e., the main chain comprises one or more sidechains).

An organic group which is “substituted with one or more substituents” means that one or more (such as 1 to the maximum number of hydrogen atoms bound to the organic group, e.g., 1, 2, 3, 4, 5, 6, 7, 8, 9, or up to 10, such as between 1 to 5, 1 to 4, or 1 to 3, or 1 or 2) hydrogen atoms of the organic group are replaced with a substituent other than hydrogen (when more than one hydrogen atom is replaced the substituents may be the same or different). Preferably, the one or more substituents may be selected from the 1^{st} level substituents, 2^{nd} level substituents, or 3^{rd} level substituents described herein.

The expression “hydrogen bond” or “H-bond” as used herein means a non-covalent bond (in some embodiments a primarily electrostatic force of attraction) between (i) a hydrogen atom which is covalently bound to a more electronegative atom or group, and (ii) a lone pair of electrons of another electronegative atom. In some embodiments, the more electronegative atom or group includes nitrogen atoms and oxygen atoms; thus, examples of groups in which a hydrogen atom is covalently bound to a more electronegative atom or group include amino groups bearing at least one covalently attached hydrogen atom, the —NH— group of amide groups, hydroxyl groups (as such (as in respective alcohols) or as part of other functional groups (e.g., as part of carboxyl (—COOH) groups)), and sulfanyl groups (as such (as in respective thiols) or as part of other functional groups (e.g., as part of disulfanyl (—SSH) or thioester (—C(OSH) groups))). In some embodiments, the lone pair of electrons of another electronegative atom is a lone pair of an oxygen atom present in a carbonyl group or a lone pair of a nitrogen atom present in a primary, secondary or tertiary amino group.

The expression “hydrogen bond donor” as used herein means an atom, ion, or a molecule component of a hydrogen bond which supplies the bridging (shared) hydrogen atom. In some embodiments, a hydrogen bond donor includes amino groups bearing at least one covalently attached hydrogen atom, the —NH— group of amide groups, hydroxyl groups (as such (as in respective alcohols) or as part of other functional groups (e.g., as part of carboxyl (—COOH) groups)), and sulfanyl groups (as such (as in respective thiols) or as part of other functional groups (e.g., as part of disulfanyl (—SSH) or thioester (—C(OSH) groups))).

The expression “hydrogen bond acceptor” as used herein means an atom, ion, or a molecule component of a hydrogen bond which does not supply the bridging (shared) hydrogen atom. In some embodiments, a hydrogen bond acceptor comprises at least one lone pair of electrons. Examples of hydrogen bond acceptors include carbonyl moieties and primary, secondary and tertiary amino groups.

The term “tocopherol” means a group of four compounds (i.e., α-tocopherol, β-tocopherol, γ-tocopherol, and δ-tocopherol) having the following formula:

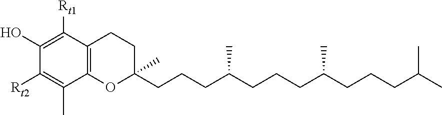

wherein each of Rₜ₁ and Rₜ₂ is independently H or methyl. In α-tocopherol, Rₜ₁ and Rₜ₂ are both methyl; in β-tocopherol, Rₜ₁ is methyl, and Rₜ₂ is H: in γ-tocopherol, Rₜ₁ is H, and Rₜ₂ is methyl; and in δ-tocopherol, Rₜ₁ and Rₜ₂ are both H. The term “tocopherol moiety” or “tocopheryl moiety” means a monovalent radical of tocopherol, preferably that in which the hydrogen atom of the hydroxy group has been removed.

The term “aqueous phase” as used herein in relation to a composition/formulation comprising particles, in particular LNPs, means the mobile or liquid phase, i.e., the continuous water phase including all components dissolved therein but (formally) excluding the particles. Thus, if particles, such as LNPs, are dispersed in an aqueous phase and the aqueous phase is to be substantially free of compound X, the aqueous phase is free of X is such manner as it is practically and realistically feasible, e.g., the concentration of compound X in the aqueous composition is less than 1% by weight. However, it is possible that, at the same time, the particles dispersed in the aqueous phase may comprise compound X in an amount of more than 1% by weight.

The expression “substantially free of X”, as used herein, means that a mixture (such as a composition or formulation described herein or an aqueous phase of such composition or formulation) is free of X is such manner as it is practically and realistically feasible. For example, if the mixture is substantially free of X, the amount of X in the mixture may be less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the mixture.

Thus, if a composition (such as an RNA LNP composition) described herein is to be substantially free of a lipid or lipid-like material comprising polyethyleneglycol (PEG), it is preferred that the amount of lipid or lipid-like material comprising PEG in the composition (such as the RNA LNP composition) is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the composition.

If the aqueous phase of a composition (such as an RNA LNP composition) described herein is to be substantially free of a lipid or lipid-like material comprising PEG, it is preferred that the amount of lipid or lipid-like material comprising PEG in aqueous phase of the composition (such as the RNA LNP composition) is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

If a composition (such as an RNA LNP composition) described herein is to be substantially free of PEG, it is preferred that the amount of PEG in the composition (such as the RNA LNP composition) is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the composition.

If the aqueous phase of a composition (such as an RNA LNP composition) described herein is to be substantially free of PEG, it is preferred that the amount of PEG in aqueous phase of the composition (such as the RNA LNP composition) is less than 1% by weight (e.g., less than 0.5% by weight, less than 0.4% by weight, less than 0.3% by weight, less than 0.2% by weight, less than 0.1% by weight, less than 0.09% by weight, less than 0.08% by weight, less than 0.07% by weight, less than 0.06% by weight, less than 0.05% by weight, less than 0.04% by weight, less than 0.03% by weight, less than 0.02% by weight, less than 0.01% by weight, less than 0.005% by weight, less than 0.001% by weight), based on the total weight of the aqueous phase.

### Nucleic Acid

The term “nucleic acid” comprises deoxyribonucleic acid (DNA), ribonucleic acid (RNA), combinations thereof, and modified forms thereof. The term comprises genomic DNA, cDNA, mRNA, recombinantly produced and chemically synthesized molecules. A nucleic acid may be present as a single-stranded or double-stranded and linear or covalently circularly closed molecule. A nucleic acid can be isolated. The term “isolated nucleic acid” means, according to the present disclosure, that the nucleic acid (i) was amplified in vitro, for example via polymerase chain reaction (PCR) for DNA or in vitro transcription (using, e.g., an RNA polymerase) for RNA, (ii) was produced recombinantly by cloning, (iii) was purified, for example, by cleavage and separation by gel electrophoresis, or (iv) was synthesized, for example, by chemical synthesis.

The term “nucleoside” (abbreviated herein as “N”) relates to compounds which can be thought of as nucleotides without a phosphate group. While a nucleoside is a nucleobase linked to a sugar (e.g., ribose or deoxyribose), a nucleotide is composed of a nucleoside and one or more phosphate groups. Examples of nucleosides include cytidine, uridine, pseudouridine, adenosine, and guanosine.

The five standard nucleosides which usually make up naturally occurring nucleic acids are uridine, adenosine, thymidine, cytidine and guanosine. The five nucleosides are commonly abbreviated to their one letter codes U, A, T, C and G, respectively. However, thymidine is more commonly written as “dT” (“d” represents “deoxy”) as it contains a 2′-deoxyribofuranose moiety rather than the ribofuranose ring found in uridine. This is because thymidine is found in deoxyribonucleic acid (DNA) and not ribonucleic acid (RNA). Conversely, uridine is found in RNA and not DNA. The remaining three nucleosides may be found in both RNA and DNA. In RNA, they would be represented as A, C and G, whereas in DNA they would be represented as dA, dC and dG.

A modified purine (A or G) or pyrimidine (C, T, or U) base moiety is preferably modified by one or more alkyl groups, more preferably one or more C₁₋₄ alkyl groups, even more preferably one or more methyl groups. Particular examples of modified purine or pyrimidine base moieties include N⁷-alkyl-guanine, N⁶-alkyl-adenine, 5-alkyl-cytosine, 5-alkyl-uracil, and N(1)-alkyl-uracil, such as N⁷—C₁₋₄ alkyl-guanine, N⁶—C₁₋₄ alkyl-adenine, 5-C₁₋₄ alkyl-cytosine, 5-C₁₋₄ alkyl-uracil, and N(1)-C₁₋₄ alkyl-uracil, preferably N⁷-methyl-guanine, No-methyl-adenine, 5-methyl-cytosine, 5-methyl-uracil, and N(1)-methyl-uracil.

### DNA

In some embodiments of the disclosure, the nucleic acid is DNA.

Herein, the term “DNA” relates to a nucleic acid molecule which includes deoxyribonucleotide residues. In certain embodiments, the DNA contains all or a majority of deoxyribonucleotide residues. As used herein, “deoxyribonucleotide” refers to a nucleotide which lacks a hydroxyl group at the 2′-position of a β-D-ribofuranosyl group. DNA encompasses without limitation, double stranded DNA, single stranded DNA, isolated DNA such as partially purified DNA, essentially pure DNA, synthetic DNA, recombinantly produced DNA, as well as modified DNA that differs from naturally occurring DNA by the addition, deletion, substitution and/or alteration of one or more nucleotides. Such alterations may refer to addition of non-nucleotide material to internal DNA nucleotides or to the end(s) of DNA. It is also contemplated herein that nucleotides in DNA may be non-standard nucleotides, such as chemically synthesized nucleotides or ribonucleotides. For the present disclosure, these altered DNAs are considered analogs of naturally-occurring DNA. A molecule contains “a majority of deoxyribonucleotide residues” if the content of deoxyribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

DNA may be recombinant DNA and may be obtained by cloning of a nucleic acid, in particular cDNA. The cDNA may be obtained by reverse transcription of RNA.

### RNA

In some embodiments of the disclosure, the nucleic acid is RNA.

According to the present disclosure, the term “RNA” means a nucleic acid molecule which includes ribonucleotide residues. In certain embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered/modified nucleotides can be referred to as analogs of naturally occurring nucleotides, and the corresponding RNAs containing such altered/modified nucleotides (i.e., altered/modified RNAs) can be referred to as analogs of naturally occurring RNAs. In some embodiments, the RNA used according to the present disclosure comprises a population of different RNA molecules, e.g. a mixture of different RNA molecules optionally encoding different peptides and/or proteins. Thus, according to the present disclosure, the term “RNA” may include a mixture of RNA molecules.

A molecule contains “a majority of ribonucleotide residues” if the content of ribonucleotide residues in the molecule is more than 50% (such as at least 55%, at least 60%, at least 65%, at least 70%, at least 75%, at least 80%, at least 85%, at least 90%, at least 95%, at least 96%, at least 97%, at least 98%, at least 99%), based on the total number of nucleotide residues in the molecule. The total number of nucleotide residues in a molecule is the sum of all nucleotide residues (irrespective of whether the nucleotide residues are standard (i.e., naturally occurring) nucleotide residues or analogs thereof).

“RNA” includes mRNA, tRNA, ribosomal RNA (rRNA), small nuclear RNA (snRNA), self-amplifying RNA (saRNA), single-stranded RNA (ssRNA), dsRNA, inhibitory RNA (such as antisense ssRNA, small interfering RNA (siRNA), or microRNA (miRNA)), activating RNA (such as small activating RNA) and immunostimulatory RNA (isRNA). In certain embodiments, the RNA used according to the present disclosure is mRNA.

In certain embodiments, the RNA comprises an open reading frame (ORF) encoding a peptide, polypeptide, or protein. Said RNA may express the encoded peptide, polypeptide, or protein. For example, said RNA may be RNA encoding and expressing a pharmaceutically active peptide or protein. In some embodiments, RNA is able to interact with the cellular translation machinery allowing translation of the peptide or protein. A cell may produce the encoded peptide or protein intracellularly (e.g. in the cytoplasm), may secrete the encoded peptide or protein, or may produce it on the surface. Alternatively, the RNA can be non-coding RNA such as antisense-RNA, micro RNA (miRNA) or SiRNA.

The term “in vitro transcription” or “IVT” as used herein means that the transcription (i.e., the generation of RNA) is conducted in a cell-free manner. I.e., IVT does not use living/cultured cells but rather the transcription machinery extracted from cells (e.g., cell lysates or the isolated components thereof, including an RNA polymerase (preferably T7, T3 or SP6 polymerase)).

According to the present disclosure, the term “mRNA” means “messenger-RNA” and includes a “transcript” which may be generated by using a DNA template. Generally, mRNA encodes a peptide or protein.

mRNA is single-stranded but may contain self-complementary sequences that allow parts of the mRNA to fold and pair with itself to form double helices.

According to the present disclosure, “dsRNA” means double-stranded RNA and is RNA with two partially or completely complementary strands.

In certain embodiments of the present disclosure, the mRNA relates to an RNA transcript which encodes a peptide or protein.

In some embodiments, the RNA which preferably encodes a peptide or protein has a length of at least 45 nucleotides (such as at least 60, at least 90, at least 100, at least 200, at least 300, at least 400, at least 500, at least 600, at least 700, at least 800, at least 900, at least 1,000, at least 1,500, at least 2,000, at least 2,500, at least 3,000, at least 3,500, at least 4,000, at least 4,500, at least 5,000, at least 6,000, at least 7,000, at least 8,000, at least 9,000 nucleotides), preferably up to 15,000, such as up to 14,000, up to 13,000, up to 12,000 nucleotides, up to 11,000 nucleotides or up to 10,000 nucleotides.

In some embodiments, the RNA (such as mRNA) contains a 5′ untranslated region (5′-UTR), a peptide/polypeptide/protein coding region and a 3′ untranslated region (3′-UTR). In some embodiments, the RNA (such as mRNA) is produced by in vitro transcription or chemical synthesis. In some embodiments, the RNA (such as mRNA) is produced by in vitro transcription using a DNA template. The in vitro transcription methodology is known to the skilled person; cf., e.g., Molecular Cloning: A Laboratory Manual, 2^{nd} Edition, J. Sambrook et al. eds., Cold Spring Harbor Laboratory Press, Cold Spring Harbor 1989. Furthermore, a variety of in vitro transcription kits is commercially available, e.g., from Thermo Fisher Scientific (such as TranscriptAid™ T7 kit, MEGAscript® T7 kit, MAXIscript®), New England BioLabs Inc. (such as HiScribe™ T7 kit, HiScribe™ T7 ARCA mRNA kit), Promega (such as RiboMAX™, HeLaScribe®, Riboprobe® systems), Jena Bioscience (such as SP6 or T7 transcription kits), and Epicentre (such as AmpliScribe™). For providing modified RNA (such as mRNA), correspondingly modified nucleotides, such as modified naturally occurring nucleotides, non-naturally occurring nucleotides and/or modified non-naturally occurring nucleotides, can be incorporated during synthesis (preferably in vitro transcription), or modifications can be effected in and/or added to the mRNA after transcription.

In some embodiments, RNA (such as mRNA) is in vitro transcribed RNA (IVT-RNA) and may be obtained by in vitro transcription of an appropriate DNA template. The promoter for controlling transcription can be any promoter for any RNA polymerase. Particular examples of RNA polymerases are the T7, T3, and SP6 RNA polymerases. Preferably, the in vitro transcription is controlled by a T7 or SP6 promoter. A DNA template for in vitro transcription may be obtained by cloning of a nucleic acid, in particular cDNA, and introducing it into an appropriate vector for in vitro transcription. The cDNA may be obtained by reverse transcription of RNA.

In certain embodiments of the present disclosure, the RNA (such as mRNA) is “replicon RNA” (such as “replicon mRNA”) or simply a “replicon”, in particular “self-replicating RNA” (such as “self-replicating mRNA”) or “self-amplifying RNA” (or “self-amplifying mRNA”). In certain embodiments, the replicon or self-replicating RNA (such as self-replicating mRNA) is derived from or comprises elements derived from an ssRNA virus, in particular a positive-stranded ssRNA virus such as an alphavirus. Alphaviruses are typical representatives of positive-stranded RNA viruses. Alphaviruses replicate in the cytoplasm of infected cells (for review of the alphaviral life cycle see José et al., Future Microbiol., 2009, vol. 4, pp. 837-856). The total genome length of many alphaviruses typically ranges between 11,000 and 12,000 nucleotides, and the genomic RNA typically has a 5′-cap, and a 3′ poly(A) tail. The genome of alphaviruses encodes non-structural proteins (involved in transcription, modification and replication of viral RNA and in protein modification) and structural proteins (forming the virus particle). There are typically two open reading frames (ORFs) in the genome. The four non-structural proteins (nsP1-nsP4) are typically encoded together by a first ORF beginning near the 5′ terminus of the genome, while alphavirus structural proteins are encoded together by a second ORF which is found downstream of the first ORF and extends near the 3′ terminus of the genome. Typically, the first ORF is larger than the second ORF, the ratio being roughly 2:1. In cells infected by an alphavirus, only the nucleic acid sequence encoding non-structural proteins is translated from the genomic RNA, while the genetic information encoding structural proteins is translatable from a subgenomic transcript, which is an RNA molecule that resembles eukaryotic messenger RNA (mRNA; Gould et al., 2010, Antiviral Res., vol. 87 pp. 111-124). Following infection, i.e. at early stages of the viral life cycle, the (+) stranded genomic RNA directly acts like a messenger RNA for the translation of the open reading frame encoding the non-structural poly-protein (nsP1234). Alphavirus-derived vectors have been proposed for delivery of foreign genetic information into target cells or target organisms. In simple approaches, the open reading frame encoding alphaviral structural proteins is replaced by an open reading frame encoding a protein of interest. Alphavirus-based trans-replication systems rely on alphavirus nucleotide sequence elements on two separate nucleic acid molecules: one nucleic acid molecule encodes a viral replicase, and the other nucleic acid molecule is capable of being replicated by said replicase in trans (hence the designation trans-replication system). Trans-replication requires the presence of both these nucleic acid molecules in a given host cell. The nucleic acid molecule capable of being replicated by the replicase in trans must comprise certain alphaviral sequence elements to allow recognition and RNA synthesis by the alphaviral replicase.

In some embodiments of the present disclosure, the RNA (such as mRNA) contains one or more modifications, e.g., in order to increase its stability and/or increase translation efficiency and/or decrease immunogenicity and/or decrease cytotoxicity. For example, in order to increase expression of the RNA (such as mRNA), it may be modified within the coding region, i.e., the sequence encoding the expressed peptide or protein, preferably without altering the sequence of the expressed peptide or protein. Such modifications are described, for example, in WO 2007/036366 and PCT/EP2019/056502, and include the following: a 5′-cap structure; an extension or truncation of the naturally occurring poly(A) tail; an alteration of the 5′- and/or 3′-untranslated regions (UTR) such as introduction of a UTR which is not related to the coding region of said RNA; the replacement of one or more naturally occurring nucleotides with synthetic nucleotides (examples of modified ribonucleotides are given below and include, without limitation, 5-methylcytidine, pseudouridine (ψ), N1-methyl-pseudouridine (m¹ψ) or 5-methyl-uridine (m⁵U)); and codon optimization (e.g., to alter, preferably increase, the GC content of the RNA). The term “modification” in the context of modified RNA (such as modified mRNA) according to the present disclosure preferably relates to any modification of an RNA (such as mRNA) which is not naturally present in said RNA (such as mRNA).

In some embodiments, the RNA (such as mRNA) comprises a 5′-cap structure. In some embodiments, the RNA (such as mRNA) does not have uncapped 5′-triphosphates. In some embodiments, the RNA (such as mRNA) may comprise a conventional 5′-cap and/or a 5′-cap analog. The term “conventional 5′-cap” refers to a cap structure found on the 5′-end of an mRNA molecule and generally consists of a guanosine 5′-triphosphate (Gppp) which is connected via its triphosphate moiety to the 5′-end of the next nucleotide of the mRNA (i.e., the guanosine is connected via a 5′ to 5′ triphosphate linkage to the rest of the mRNA). The guanosine may be methylated at position N⁷ (resulting in the cap structure m⁷Gppp). The term “5′-cap analog” refers to a 5′-cap which is based on a conventional 5′-cap but which has been modified at either the 2′- or 3′-position of the m′guanosine structure in order to avoid an integration of the 5′-cap analog in the reverse orientation (such 5′-cap analogs are also called anti-reverse cap analogs (ARCAs)). Particularly preferred 5′-cap analogs are those having one or more substitutions at the bridging and non-bridging oxygen in the phosphate bridge, such as phosphorothioate modified 5′-cap analogs at the β-phosphate (such as m₂^{7,2′O}G(5′)ppSp(5′)G (referred to as beta-S-ARCA or β-S-ARCA)), as described in PCT/EP2019/056502. Providing an RNA (such as mRNA) with a 5′-cap structure as described herein may be achieved by in vitro transcription of a DNA template in presence of a corresponding 5′-cap compound, wherein said 5′-cap structure is co-transcriptionally incorporated into the generated RNA (such as mRNA) strand, or the RNA (such as mRNA) may be generated, for example, by in vitro transcription, and the 5′-cap structure may be attached to the mRNA post-transcriptionally using capping enzymes, for example, capping enzymes of vaccinia virus.

In some embodiments, the RNA (such as mRNA) comprises a 5′-cap structure selected from the group consisting of m₂^{7,2′O}G(5′)ppSp(5′)G (in particular its D1 diastereomer), m₂^{7,3′O}G(5′)ppp(5′)G, and m₂^{7,3′-O}-OGppp(m₁^{2′-O})ApG. In some embodiments, RNA encoding a peptide, polypeptide, or protein comprising an antigen or epitope comprises m₂^{7,2′O}G(5′)ppSp(5′)G (in particular its D1 diastereomer) as 5′-cap structure.

In some embodiments, the RNA (such as mRNA) comprises a cap0, cap1, or cap2, preferably cap1 or cap2. According to the present disclosure, the term “cap0” means the structure “m⁷GpppN”, wherein N is any nucleoside bearing an OH moiety at position 2′. According to the present disclosure, the term “cap1” means the structure “m⁷GpppNm”, wherein Nm is any nucleoside bearing an OCH₃ moiety at position 2′. According to the present disclosure, the term “cap2” means the structure “m⁷GpppNmNm”, wherein each Nm is independently any nucleoside bearing an OCH₃ moiety at position 2′.

The 5′-cap analog beta-S-ARCA (β-S-ARCA) has the following structure:

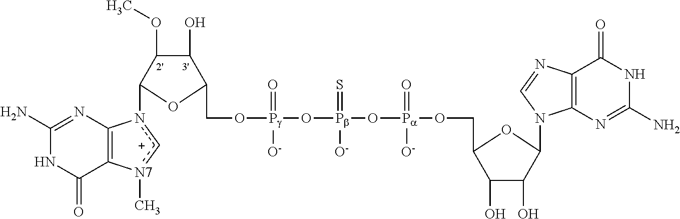

The “D1 diastereomer of beta-S-ARCA” or “beta-S-ARCA (D1)” is the diastereomer of beta-S-ARCA which elutes first on an HPLC column compared to the D2 diastereomer of beta-S-ARCA (beta-S-ARCA (D2)) and thus exhibits a shorter retention time. The HPLC preferably is an analytical HPLC. In some embodiments, a Supelcosil LC-18-T RP column, preferably of the format: 5 μm, 4.6×250 mm is used for separation, whereby a flow rate of 1.3 ml/min can be applied. In some embodiments, a gradient of methanol in ammonium acetate, for example, a 0-25% linear gradient of methanol in 0.05 M ammonium acetate, pH=5.9, within 15 min is used. UV-detection (VWD) can be performed at 260 nm and fluorescence detection (FLD) can be performed with excitation at 280 nm and detection at 337 nm.

The 5′-cap analog m₂^{7,3′-O}Gppp(m₁^{2′-O})ApG (also referred to as m₂^{7,3′O}G(5′)ppp(5′)m^{2′-O}ApG) which is a building block of a cap1 has the following structure:

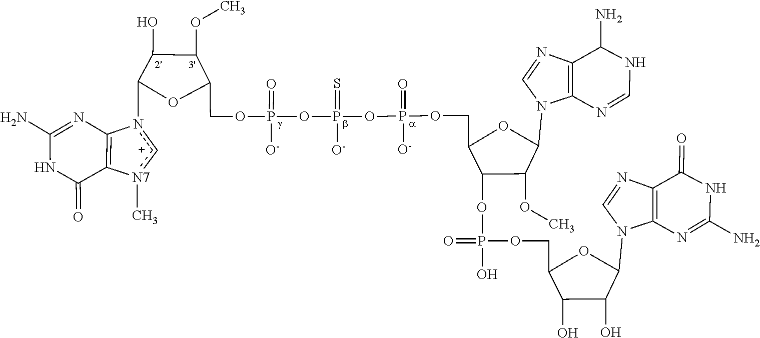

An exemplary cap0 mRNA comprising β-S-ARCA and mRNA has the following structure:

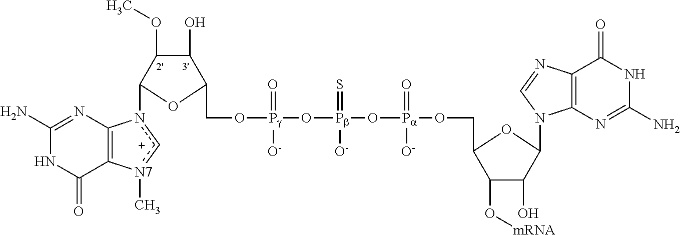

An exemplary cap0 mRNA comprising m₂^{7,30}G(5′)ppp(5′)G and mRNA has the following structure:

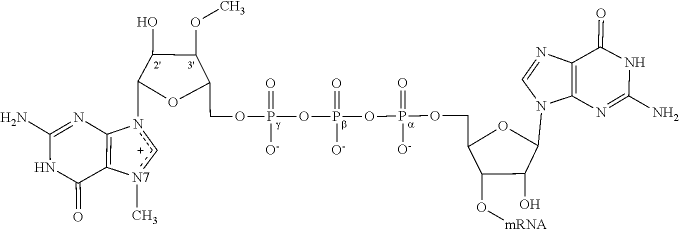

An exemplary cap1 mRNA comprising m₂^{7,3′-O}Gppp(m₁^{2′-O})ApG and mRNA has the following structure:

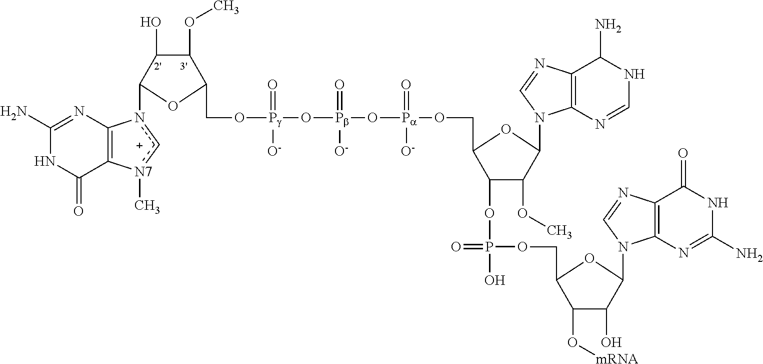

In some embodiments, the RNA (such as mRNA) comprises a 3′-poly(A) sequence. As used herein, the term “poly-A tail” or “poly-A sequence” refers to an uninterrupted or interrupted sequence of adenylate residues which is typically located at the 3′-end of an RNA (such as mRNA) molecule. Poly-A tails or poly-A sequences are known to those of skill in the art and may follow the 3′-UTR in the RNAs described herein. An uninterrupted poly-A tail is characterized by consecutive adenylate residues. In nature, an uninterrupted poly-A tail is typical. RNAs (such as mRNAs) disclosed herein can have a poly-A tail attached to the free 3′-end of the RNA by a template-independent RNA polymerase after transcription or a poly-A tail encoded by DNA and transcribed by a template-dependent RNA polymerase.

It has been demonstrated that a poly-A tail of about 120 A nucleotides has a beneficial influence on the levels of mRNA in transfected eukaryotic cells, as well as on the levels of protein that is translated from an open reading frame that is present upstream (5′) of the poly-A tail (Holtkamp et al., 2006, Blood, vol. 108, pp. 4009-4017).

The poly-A tail may be of any length. In some embodiments, a poly-A tail comprises, essentially consists of, or consists of at least 20, at least 30, at least 40, at least 80, or at least 100 and up to 500, up to 400, up to 300, up to 200, or up to 150 A nucleotides, and, in particular, about 120 A nucleotides. In this context, “essentially consists of” means that most nucleotides in the poly-A tail, typically at least 75%, at least 80%, at least 85%, at least 90%, at least 95%, at least 96%, at least 97%, at least 98%, or at least 99% by number of nucleotides in the poly-A tail are A nucleotides, but permits that remaining nucleotides are nucleotides other than A nucleotides, such as U nucleotides (uridylate), G nucleotides (guanylate), or C nucleotides (cytidylate). In this context, “consists of” means that all nucleotides in the poly-A tail, i.e., 100% by number of nucleotides in the poly-A tail, are A nucleotides. The term “A nucleotide” or “A” refers to adenylate.

In some embodiments, a poly-A tail is attached during RNA transcription, e.g., during preparation of in vitro transcribed RNA, based on a DNA template comprising repeated dT nucleotides (deoxythymidylate) in the strand complementary to the coding strand. The DNA sequence encoding a poly-A tail (coding strand) is referred to as poly(A) cassette.

In some embodiments, the poly(A) cassette present in the coding strand of DNA essentially consists of dA nucleotides, but is interrupted by a random sequence of the four nucleotides (dA, dC, dG, and dT).

Such random sequence may be 5 to 50, 10 to 30, or 10 to 20 nucleotides in length. Such a cassette is disclosed in WO 2016/005324 A1, hereby incorporated by reference. Any poly(A) cassette disclosed in WO 2016/005324 A1 may be used in the present disclosure. A poly(A) cassette that essentially consists of dA nucleotides, but is interrupted by a random sequence having an equal distribution of the four nucleotides (dA, dC, dG, dT) and having a length of e.g., 5 to 50 nucleotides shows, on DNA level, constant propagation of plasmid DNA in E. coli and is still associated, on RNA level, with the beneficial properties with respect to supporting RNA stability and translational efficiency is encompassed. Consequently, in some embodiments, the poly-A tail contained in an mRNA molecule described herein essentially consists of A nucleotides, but is interrupted by a random sequence of the four nucleotides (A, C, G, U). Such random sequence may be 5 to 50, 10 to 30, or 10 to 20 nucleotides in length.

In some embodiments, no nucleotides other than A nucleotides flank a poly-A tail at its 3′-end, i.e., the poly-A tail is not masked or followed at its 3′-end by a nucleotide other than A.

In some embodiments, a poly-A tail may comprise at least 20, at least 30, at least 40, at least 80, or at least 100 and up to 500, up to 400, up to 300, up to 200, or up to 150 nucleotides. In some embodiments, the poly-A tail may essentially consist of at least 20, at least 30, at least 40, at least 80, or at least 100 and up to 500, up to 400, up to 300, up to 200, or up to 150 nucleotides. In some embodiments, the poly-A tail may consist of at least 20, at least 30, at least 40, at least 80, or at least 100 and up to 500, up to 400, up to 300, up to 200, or up to 150 nucleotides. In some embodiments, the poly-A tail comprises at least 100 nucleotides, such as at least 120 nucleotides. In some embodiments, the poly-A tail comprises about 150 nucleotides. In some embodiments, the poly-A tail comprises about 120 nucleotides.

In some embodiments, RNA (such as mRNA) used in present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′-end, upstream of the start codon of a protein-encoding region. A 5′-UTR is downstream of the 5′-cap (if present), e.g., directly adjacent to the 5′-cap. A 3′-UTR, if present, is located at the 3′-end, downstream of the termination codon of a protein-encoding region, but the term “3′-UTR” does preferably not include the poly-A sequence. Thus, the 3′-UTR is upstream of the poly-A sequence (if present), e.g., directly adjacent to the poly-A sequence. Incorporation of a 3′-UTR into the 3′-non translated region of an RNA (preferably mRNA) molecule can result in an enhancement in translation efficiency. A synergistic effect may be achieved by incorporating two or more of such 3′-UTRs (which are preferably arranged in a head-to-tail orientation; cf., e.g., Holtkamp et al., Blood 108, 4009-4017 (2006)). The 3′-UTRs may be autologous or heterologous to the RNA (preferably mRNA) into which they are introduced. In certain embodiments, the 3′-UTR is derived from a globin gene or mRNA, such as a gene or mRNA of alpha2-globin, alpha1-globin, or beta-globin, preferably beta-globin, more preferably human beta-globin. For example, the RNA (preferably mRNA) may be modified by the replacement of the existing 3′-UTR with or the insertion of one or more, preferably two copies of a 3′-UTR derived from a globin gene, such as alpha2-globin, alpha1-globin, beta-globin, preferably beta-globin, more preferably human beta-globin.

The RNA (such as mRNA) may have modified ribonucleotides in order to increase its stability and/or decrease immunogenicity and/or decrease cytotoxicity. For example, in some embodiments, uridine in the RNA (such as mRNA) described herein is replaced (partially or completely, preferably completely) by a modified nucleoside. In some embodiments, the modified nucleoside is a modified uridine.

In some embodiments, the modified uridine replacing uridine is selected from the group consisting of pseudouridine (ψ), N¹-methyl-pseudouridine (m1ψ), 5-methyl-uridine (m5U), and combinations thereof.

In some embodiments, the modified nucleoside replacing (partially or completely, preferably completely) uridine in the RNA (such as mRNA) may be any one or more of 3-methyl-uridine (m³U), 5-methoxy-uridine (mo5U), 5-aza-uridine, 6-aza-uridine, 2-thio-5-aza-uridine, 2-thio-uridine (s2U), 4-thio-uridine (s4U), 4-thio-pseudouridine, 2-thio-pseudouridine, 5-hydroxy-uridine (ho5U), 5-aminoallyl-uridine, 5-halo-uridine (e.g., 5-iodo-uridineor 5-bromo-uridine), uridine 5-oxyacetic acid (cmo5U), uridine 5-oxyacetic acid methyl ester (memo5U), 5-carboxymethyl-uridine (cm5U), 1-carboxymethyl-pseudouridine, 5-carboxyhydroxymethyl-uridine (chm5U), 5-carboxyhydroxymethyl-uridine methyl ester (mchm5U), 5-methoxycarbonylmethyl-uridine (mcm5U), 5-methoxycarbonylmethyl-2-thio-uridine (mcm5s2U), 5-aminomethyl-2-thio-uridine (nm5s2U), 5-methylaminomethyl-uridine (mnm5U), 1-ethyl-pseudouridine, 5-methylaminomethyl-2-thio-uridine (mnm5s2U), 5-methylaminomethyl-2-seleno-uridine c (mnm5se2U), 5-carbamoylmethyl-uridine (ncm5U), 5-carboxymethylaminomethyl-uridine (cmnm5U), 5-carboxymethylaminomethyl-2-thio-uridine (cmnm5s2U), 5-propynyl-uridine, 1-propynyl-pseudouridine, 5-taurinomethyl-uridine (tm5U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s2U), 1-methyl-4-thio-pseudouridine (m1s4ψ), 4-thio-1-methyl-pseudouridine, 3-methyl-pseudouridine (m3ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp3U), 1-methyl-3-(3-amino-3-carboxypropyl) pseudouridine (acp3 ψ), 5-(isopentenylaminomethyl) uridine (inm5U), 5-(isopentenylaminomethyl)-2-thio-uridine (inm5s2U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s2Um), 5-methoxycarbonylmethyl-2′-O-methyl-uridine (mcm5Um), 5-carbamoylmethyl-2′-O-methyl-uridine (ncm5Um), 5-carboxymethylaminomethyl-2′-O-methyl-uridine (cmnm5Um), 3,2′-O-dimethyl-uridine (m3Um), 5-(isopentenylaminomethyl)-2′-O-methyl-uridine (inm5Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino) uridine, or any other modified uridine known in the art.

An RNA (preferably mRNA) which is modified by pseudouridine (replacing partially or completely, preferably completely, uridine) is referred to herein as “Ψ-modified”, whereas the term “m1Ψ-modified” means that the RNA (preferably mRNA) contains N(1)-methylpseudouridine (replacing partially or completely, preferably completely, uridine). Furthermore, the term “m⁵U-modified” means that the RNA (preferably mRNA) contains 5-methyluridine (replacing partially or completely, preferably completely, uridine). Such Ψ- or m1Ψ- or m⁵U-modified RNAs usually exhibit decreased immunogenicity compared to their unmodified forms and, thus, are preferred in applications where the induction of an immune response is to be avoided or minimized.

The codons of the RNA (preferably mRNA) used in the present disclosure may further be optimized, e.g., to increase the GC content of the RNA and/or to replace codons which are rare in the cell (or subject) in which the peptide or protein of interest is to be expressed by codons which are synonymous frequent codons in said cell (or subject). In some embodiments, the amino acid sequence encoded by the RNA used in the present disclosure is encoded by a coding sequence which is codon-optimized and/or the G/C content of which is increased compared to wild type coding sequence. This also includes embodiments, wherein one or more sequence regions of the coding sequence are codon-optimized and/or increased in the G/C content compared to the corresponding sequence regions of the wild type coding sequence. In some embodiments, the codon-optimization and/or the increase in the G/C content preferably does not change the sequence of the encoded amino acid sequence.

The term “codon-optimized” refers to the alteration of codons in the coding region of a nucleic acid (in particular RNA) molecule to reflect the typical codon usage of a host organism without preferably altering the amino acid sequence encoded by the nucleic acid molecule. Within the context of the present disclosure, coding regions are preferably codon-optimized for optimal expression in a subject to be treated using the RNA (preferably mRNA) described herein. Codon-optimization is based on the finding that the translation efficiency is also determined by a different frequency in the occurrence of tRNAs in cells. Thus, the sequence of RNA (preferably mRNA) may be modified such that codons for which frequently occurring tRNAs are available are inserted in place of “rare codons”.

In some embodiments, the guanosine/cytosine (G/C) content of the coding region of the RNA (preferably mRNA) described herein is increased compared to the G/C content of the corresponding coding sequence of the wild type RNA, wherein the amino acid sequence encoded by the RNA (preferably mRNA) is preferably not modified compared to the amino acid sequence encoded by the wild type RNA. This modification of the RNA sequence is based on the fact that the sequence of any RNA region to be translated is important for efficient translation of that RNA (preferably mRNA). Sequences having an increased G (guanosine)/C (cytosine) content are more stable than sequences having an increased A (adenosine)/U (uracil) content. In respect to the fact that several codons code for one and the same amino acid (so-called degeneration of the genetic code), the most favorable codons for the stability can be determined (so-called alternative codon usage). Depending on the amino acid to be encoded by the RNA (preferably mRNA), there are various possibilities for modification of the RNA sequence, compared to its wild type sequence. In particular, codons which contain A and/or U nucleotides can be modified by substituting these codons by other codons, which code for the same amino acids but contain no A and/or U or contain a lower content of A and/or U nucleotides.

In various embodiments, the G/C content of the coding region of the mRNA described herein is increased by at least 10%, at least 20%, at least 30%, at least 40%, at least 50%, at least 55%, or even more compared to the G/C content of the coding region of the wild type RNA.

A combination of the above described modifications, i.e., incorporation of a 5′-cap structure, incorporation of a poly-A sequence, unmasking of a poly-A sequence, alteration of the 5′- and/or 3′-UTR (such as incorporation of one or more 3′-UTRs), replacing one or more naturally occurring nucleotides with synthetic nucleotides (e.g., 5-methylcytidine for cytidine and/or pseudouridine (Ψ) or N(1)-methylpseudouridine (m1Ψ) or 5-methyluridine (m⁵U) for uridine), and codon optimization, has a synergistic influence on the stability of RNA (preferably mRNA) and increase in translation efficiency. Thus, in certain embodiments, the RNA (preferably mRNA) used in the present disclosure, in particular an RNA (preferably mRNA) encoding an antigen or epitope for inducing an immune response disclosed herein, contains a combination of at least two, at least three, at least four or all five of the above-mentioned modifications, i.e., (i) incorporation of a 5′-cap structure, (ii) incorporation of a poly-A sequence, unmasking of a poly-A sequence; (iii) alteration of the 5′- and/or 3′-UTR (such as incorporation of one or more 3′-UTRs); (iv) replacing one or more naturally occurring nucleotides with synthetic nucleotides (e.g., 5-methylcytidine for cytidine and/or pseudouridine (Ψ) or N(1)-methylpseudouridine (m 1 Ψ) or 5-methyluridine (m5U) for uridine), and (v) codon optimization. In some embodiments, the RNA comprises a cap1 or cap2, preferably a cap1 structure. In some embodiments, the poly-A sequence comprises at least 100 nucleotides.

Some aspects of the disclosure involve the targeted delivery of the RNA (preferably mRNA) disclosed herein to certain cells or tissues. In some embodiments, the disclosure involves targeting the lymphatic system, in particular secondary lymphoid organs, more specifically spleen. Targeting the lymphatic system, in particular secondary lymphoid organs, more specifically spleen is in particular preferred if the RNA (preferably mRNA) administered is RNA (preferably mRNA) encoding an antigen or epitope for inducing an immune response. In some embodiments, the target cell is a spleen cell. In some embodiments, the target cell is an antigen presenting cell such as a professional antigen presenting cell in the spleen. In some embodiments, the target cell is a dendritic cell in the spleen. The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naive lymphocytes and initiate an adaptive immune response.

Lipid-based RNA (such as mRNA) delivery systems have an inherent preference to the liver. Liver accumulation is caused by the discontinuous nature of the hepatic vasculature or the lipid metabolism (liposomes and lipid or cholesterol conjugates). In some embodiments, the target organ is liver and the target tissue is liver tissue. The delivery to such target tissue is preferred, in particular, if presence of mRNA or of the encoded peptide or protein in this organ or tissue is desired and/or if it is desired to express large amounts of the encoded peptide or protein and/or if systemic presence of the encoded peptide or protein, in particular in significant amounts, is desired or required.

In some embodiments, after administration of the nucleic acid (such as DNA or RNA) compositions (e.g., RNA LNP compositions) described herein, at least a portion of the nucleic acid (such as DNA or RNA) is delivered to a target cell or target organ. In some embodiments, at least a portion of the nucleic acid (such as DNA or RNA) is delivered to the cytosol of the target cell. In some embodiments, the nucleic acid is nucleic acid (e.g., DNA or RNA, preferably mRNA) encoding a peptide or protein and the nucleic acid is expressed (e.g., the RNA is translated) by the target cell to produce the peptide or protein. In some embodiments, the target cell is a cell in the liver. In some embodiments, the target cell is a muscle cell. In some embodiments, the target cell is an endothelial cell. In some embodiments, the target cell is a tumor cell or a cell in the tumor microenvironment. In some embodiments, the target cell is a blood cell. In some embodiments, the target cell is a cell in the lymph nodes. In some embodiments, the target cell is a cell in the lung. In some embodiments, the target cell is a blood cell. In some embodiments, the target cell is a cell in the skin. In some embodiments, the target cell is a spleen cell. In some embodiments, the target cell is an antigen presenting cell such as a professional antigen presenting cell in the spleen. In some embodiments, the target cell is a dendritic cell in the spleen. In some embodiments, the target cell is a T cell. In some embodiments, the target cell is a B cell. In some embodiments, the target cell is a NK cell. In some embodiments, the target cell is a monocyte. Thus, nucleic acid (such as DNA or RNA) compositions (e.g., RNA LNP compositions) described herein may be used for delivering nucleic acid (such as DNA or RNA, preferably mRNA) to such target cell. Accordingly, the present disclosure also relates to a method for delivering nucleic acid (such as DNA or RNA, preferably mRNA) to a target cell in a subject comprising the administration of one or more of the nucleic acid (such as DNA or RNA) compositions (e.g., RNA LNP compositions) described herein to the subject. In some embodiments, the nucleic acid (such as DNA or RNA) is delivered to the cytosol of the target cell. In some embodiments, the nucleic acid (such as DNA or RNA) is nucleic acid (such as DNA or RNA, preferably mRNA) encoding a peptide or protein and the nucleic acid is expressed (e.g., the RNA is translated) by the target cell to produce the peptide or protein.

### Pharmaceutically Active Peptides or Polypeptides

“Encoding” refers to the inherent property of specific sequences of nucleotides in a polynucleotide, such as a gene, a cDNA, or an RNA (preferably mRNA), to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of RNA (preferably mRNA) corresponding to that gene produces the protein in a cell or other biological system. Both the coding strand, the nucleotide sequence of which is identical to the RNA sequence and is usually provided in sequence listings, and the non-coding strand, used as the template for transcription of a gene or cDNA, can be referred to as encoding the protein or other product of that gene or cDNA.

In some embodiments, nucleic acid (such as DNA or RNA, preferably mRNA) used in the present disclosure comprises a nucleic acid sequence (e.g., an ORF) encoding one or more polypeptides, e.g., a peptide or protein, preferably a pharmaceutically active peptide or protein.

In certain embodiments, nucleic acid (such as DNA or RNA, preferably mRNA) used in the present disclosure comprises a nucleic acid sequence (e.g., an ORF) encoding a peptide or protein, preferably a pharmaceutically active peptide or protein, and is capable of expressing said peptide or protein, in particular if transferred into a cell or subject. Thus, in some embodiments, the nucleic acid (such as DNA or RNA, preferably mRNA) used in the present disclosure contains a coding region (ORF) encoding a peptide or protein, preferably encoding a pharmaceutically active peptide or protein. In this respect, an “open reading frame” or “ORF” is a continuous stretch of codons beginning with a start codon and ending with a stop codon. Such nucleic acid (such as DNA or RNA, preferably mRNA) encoding a pharmaceutically active peptide or protein (and preferably having one or more pharmaceutical activities such as those described for pharmaceutically active proteins (e.g., immunostimulatory activity)) is also referred to herein as “pharmaceutically active nucleic acid” (such “pharmaceutically active RNA” (or “pharmaceutically active mRNA”)). In some embodiments, nucleic acid such as mRNA used in the present disclosure comprises a nucleic acid sequence encoding more than one peptide, polypeptide, or protein, e.g., two, three, four or more peptides, polypeptides, or proteins.

The term “pharmaceutically active RNA” also encompasses RNA which is pharmaceutically active in its own; thus, pharmaceutically active RNA may alternatively be non-coding RNA such as antisense-RNA, micro RNA (miRNA), siRNA, one or more strands of RNA interference (RNAi), short hairpin RNAs (shRNAs), or precursor of a siRNA or microRNA-like RNA, targeted to a target transcript, e.g., a transcript of an endogenous disease-related transcript of a subject.

According to the present disclosure, the term “pharmaceutically active peptide or protein” means a peptide or protein that can be used in the treatment of an individual where the expression of a peptide or protein would be of benefit, e.g., in ameliorating the symptoms of a disease or disorder. Preferably, a pharmaceutically active peptide or protein has curative or palliative properties and may be administered to ameliorate, relieve, alleviate, reverse, delay onset of or lessen the severity of one or more symptoms of a disease or disorder. Preferably, a pharmaceutically active peptide or protein has a positive or advantageous effect on the condition or disease state of an individual when administered to the individual in a therapeutically effective amount. A pharmaceutically active peptide or protein may have prophylactic properties and may be used to delay the onset of a disease or disorder or to lessen the severity of such disease or disorder. The term “pharmaceutically active peptide or protein” includes entire proteins or polypeptides, and can also refer to pharmaceutically active fragments thereof. It can also include pharmaceutically active variants and/or analogs of a peptide or protein. The terms “pharmaceutically active peptide or protein” and “therapeutic protein” are used interchangeable herein.

Specific examples of pharmaceutically active peptides and proteins include, but are not limited to, immunostimulants, e.g., cytokines, hormones, adhesion molecules, immunoglobulins, immunologically active compounds, growth factors, protease inhibitors, enzymes, receptors, apoptosis regulators, transcription factors, tumor suppressor proteins, structural proteins, reprogramming factors, genomic engineering proteins, and blood proteins.

An “immunostimulant” is any substance that stimulates the immune system by inducing activation or increasing activity of any of the immune system's components, in particular immune effector cells. The immunostimulant may be pro-inflammatory (e.g., when treating infections or cancer), or anti-inflammatory (e.g., when treating autoimmune diseases).

In some embodiments, the immunostimulant is a cytokine or a variant thereof. Examples of cytokines include interferons, such as interferon-alpha (IFN-α) or interferon-gamma (IFN-γ), interleukins, such as IL2, IL7, IL12, IL15 and IL23, colony stimulating factors, such as M-CSF and GM-CSF, and tumor necrosis factor. In some other embodiments, the immunostimulant includes an adjuvant-type immunostimulatory agent such as APC Toll-like Receptor agonists or costimulatory/cell adhesion membrane proteins. Examples of Toll-like Receptor agonists include costimulatory/adhesion proteins such as CD80, CD86, and ICAM-1.

The term “cytokines” relates to proteins which have a molecular weight of about 5 to 60 kDa (such as about 5 to 20 kDa) and which participate in cell signaling (e.g., paracrine, endocrine, and/or autocrine signaling). In particular, when released, cytokines exert an effect on the behavior of cells around the place of their release. Examples of cytokines include lymphokines, interleukins, chemokines, interferons, and tumor necrosis factors (TNFs). According to the present disclosure, cytokines do not include hormones or growth factors. Cytokines differ from hormones in that (i) they usually act at much more variable concentrations than hormones and (ii) generally are made by a broad range of cells (nearly all nucleated cells can produce cytokines). Interferons are usually characterized by antiviral, antiproliferative and immunomodulatory activities. Interferons are proteins that alter and regulate the transcription of genes within a cell by binding to interferon receptors on the regulated cell's surface, thereby preventing viral replication within the cells. The interferons can be grouped into two types.

Particular examples of cytokines include erythropoietin (EPO), colony stimulating factor (CSF), granulocyte colony stimulating factor (G-CSF), granulocyte-macrophage colony stimulating factor (GM-CSF), tumor necrosis factor (TNF), bone morphogenetic protein (BMP), interferon alfa (IFNα), interferon beta (IFNβ), interferon gamma (INFy), interleukin 2 (IL-2), interleukin 4 (IL-4), interleukin 10 (IL-10), interleukin 11 (IL-11), interleukin 12 (IL-12), and interleukin 21 (IL-21), as well as variants and derivatives thereof.

According to the disclosure, a cytokine may be a naturally occurring cytokine or a functional fragment or variant thereof. A cytokine may be human cytokine and may be derived from any vertebrate, especially any mammal. One particularly preferred cytokine is interferon-α.

Immunostimulants may be provided to a subject by administering to the subject nucleic acid (such as DNA or RNA) encoding an immunostimulant in a formulation for preferential delivery of nucleic acid (such as DNA or RNA) to liver or liver tissue. The delivery of nucleic acid (such as DNA or RNA) to such target organ or tissue is preferred, in particular, if it is desired to express large amounts of the immunostimulant and/or if systemic presence of the immunostimulant, in particular in significant amounts, is desired or required.

Nucleic acid (such as DNA or RNA) delivery systems have an inherent preference to the liver. This pertains to lipid-based particles, cationic and neutral nanoparticles, in particular lipid nanoparticles.

Examples of suitable immunostimulants for targeting liver are cytokines involved in T cell proliferation and/or maintenance. Examples of suitable cytokines include IL2 or IL7, fragments and variants thereof, and fusion proteins of these cytokines, fragments and variants, such as extended-PK cytokines.

In some embodiments, nucleic acid (such as DNA or RNA) encoding an immunostimulant may be administered in a formulation for preferential delivery of nucleic acid (such as DNA or RNA) to the lymphatic system, in particular secondary lymphoid organs, more specifically spleen. The delivery of an immunostimulant to such target tissue is preferred, in particular, if presence of the immunostimulant in this organ or tissue is desired (e.g., for inducing an immune response, in particular in case immunostimulants such as cytokines are required during T-cell priming or for activation of resident immune cells), while it is not desired that the immunostimulant is present systemically, in particular in significant amounts (e.g., because the immunostimulant has systemic toxicity).

Examples of suitable immunostimulants are cytokines involved in T cell priming. Examples of suitable cytokines include IL12, IL15, IFN-α, or IFN-β, fragments and variants thereof, and fusion proteins of these cytokines, fragments and variants, such as extended-PK cytokines.

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses.

Based on the type of receptor through which they signal, interferons are typically divided among three classes: type I interferon, type II interferon, and type III interferon.

All type I interferons bind to a specific cell surface receptor complex known as the IFN-α/β receptor (IFNAR) that consists of IFNAR1 and IFNAR2 chains.

The type I interferons present in humans are IFNα, IFNβ, IFNε, IFNκ and IFNω. In general, type I interferons are produced when the body recognizes a virus that has invaded it. They are produced by fibroblasts and monocytes. Once released, type I interferons bind to specific receptors on target cells, which leads to expression of proteins that will prevent the virus from producing and replicating its RNA and DNA.

The IFNα proteins are produced mainly by plasmacytoid dendritic cells (pDCs). They are mainly involved in innate immunity against viral infection. The genes responsible for their synthesis come in 13 subtypes that are called IFNA1, IFNA2, IFNA4, IFNA5, IFNA6, IFNA7, IFNA8, IFNA10, IFNA13, IFNA14, IFNA16, IFNA17, IFNA21. These genes are found together in a cluster on chromosome 9.

The IFNβ proteins are produced in large quantities by fibroblasts. They have antiviral activity that is involved mainly in innate immune response. Two types of IFNβ have been described, IFNβ1 and IFNβ3. The natural and recombinant forms of IFNβI have antiviral, antibacterial, and anticancer properties.

Type II interferon (IFNγ in humans) is also known as immune interferon and is activated by IL12. Furthermore, type II interferons are released by cytotoxic T cells and T helper cells.

Type III interferons signal through a receptor complex consisting of IL10R² (also called CRF2-4) and IFNLR1 (also called CRF2-12). Although discovered more recently than type I and type II IFNs, recent information demonstrates the importance of type III IFNs in some types of virus or fungal infections.

In general, type I and II interferons are responsible for regulating and activating the immune response.

According to the disclosure, a type I interferon is preferably IFNα or IFNβ, more preferably IFNα.

According to the disclosure, an interferon may be a naturally occurring interferon or a functional fragment or variant thereof. An interferon may be human interferon and may be derived from any vertebrate, especially any mammal.

Interleukins (ILs) are a group of cytokines (secreted proteins and signal molecules) that can be divided into four major groups based on distinguishing structural features. However, their amino acid sequence similarity is rather weak (typically 15-25% identity). The human genome encodes more than 50 interleukins and related proteins.

According to the disclosure, an interleukin may be a naturally occurring interleukin or a functional fragment or variant thereof. An interleukin may be human interleukin and may be derived from any vertebrate, especially any mammal.

Immunostimulant polypeptides described herein can be prepared as fusion or chimeric polypeptides that include an immunostimulant portion and a heterologous polypeptide (i.e., a polypeptide that is not an immunostimulant). The immunostimulant may be fused to an extended-PK group, which increases circulation half-life. Non-limiting examples of extended-PK groups are described infra. It should be understood that other PK groups that increase the circulation half-life of immunostimulants such as cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16 (7): 903-15 which is herein incorporated by reference in its entirety. As used herein, an “extended-PK” immunostimulant refers to an immunostimulant moiety in combination with an extended-PK group. In some embodiments, the extended-PK immunostimulant is a fusion protein in which an immunostimulant moiety is linked or fused to an extended-PK group.

In certain embodiments, the serum half-life of an extended-PK immunostimulant is increased relative to the immunostimulant alone (i.e., the immunostimulant not fused to an extended-PK group). In certain embodiments, the serum half-life of the extended-PK immunostimulant is at least 20%, at least 40%, at least 60%, at least 80%, at least 100%, at least 120%, at least 150%, at least 180%, at least 200%, at least 400%, at least 600%, at least 800%, or at least 1000% longer relative to the serum half-life of the immunostimulant alone. In certain embodiments, the serum half-life of the extended-PK immunostimulant is at least 1.5-fold, 2-fold, 2.5-fold, 3-fold, 3.5-fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the immunostimulant alone. In certain embodiments, the serum half-life of the extended-PK immunostimulant is at least 10 hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or polypeptide to reduce by 50%, in vivo, for example due to degradation and/or clearance or sequestration by natural mechanisms. An extended-PK immunostimulant suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2^{nd} Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular an immunostimulant. The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In some embodiments, an albumin fusion protein is processed by a host cell such as a cell of the target organ for administered nucleic acid (such as DNA or RNA), e.g. a liver cell, and secreted into the circulation. Processing of the nascent albumin fusion protein that occurs in the secretory pathways of the host cell used for expression of the nucleic acid (such as DNA or RNA) may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by nucleic acid (such as DNA or RNA) in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In certain embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789. The terms, human serum albumin (HSA) and human albumin (HA) are used interchangeably herein. The terms, “albumin” and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In certain embodiments, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used. In other embodiments, the albumin fusion protein has a therapeutic protein fused to both the N-terminus and the C-terminus of albumin. In certain embodiments, the therapeutic proteins fused at the N- and C-termini are the same therapeutic proteins. In certain embodiments, the therapeutic proteins fused at the N- and C-termini are different therapeutic proteins. In some embodiments, the different therapeutic proteins are both cytokines.

In some embodiments, the therapeutic protein(s) is (are) joined to the albumin through (a) peptide linker(s). A peptide linker between the fused portions may provide greater physical separation between the moieties and thus maximize the accessibility of the therapeutic protein portion, for instance, for binding to its cognate receptor. The peptide linker may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources. In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US2005/0287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in U.S. Pat. Nos. 7,176,278 and 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012/0094909. A non-limiting example of a Fn3-based extended-PK group is Fn3 (HSA), i.e., a Fn3 protein that binds to human serum albumin.

In certain aspects, the extended-PK immunostimulant, suitable for use according to the disclosure, can employ one or more peptide linkers. As used herein, the term “peptide linker” refers to a peptide or polypeptide sequence which connects two or more domains (e.g., the extended-PK moiety and an immunostimulant moiety) in a linear amino acid sequence of a polypeptide chain. For example, peptide linkers may be used to connect an immunostimulant moiety to a HSA domain.

Linkers suitable for fusing the extended-PK group to, e.g., an immunostimulant are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In some embodiments, a pharmaceutically active peptide or protein comprises a replacement protein. In this embodiment, the present disclosure provides a method for treatment of a subject having a disorder requiring protein replacement (e.g., protein deficiency disorders) comprising administering to the subject nucleic acid (such as DNA or RNA, in particular mRNA) as described herein encoding a replacement protein. The term “protein replacement” refers to the introduction of a protein (including functional variants thereof) into a subject having a deficiency in such protein. The term also refers to the introduction of a protein into a subject otherwise requiring or benefiting from providing a protein, e.g., suffering from protein insufficiency. The term “disorder characterized by a protein deficiency” refers to any disorder that presents with a pathology caused by absent or insufficient amounts of a protein. This term encompasses protein folding disorders, i.e., conformational disorders, that result in a biologically inactive protein product. Protein insufficiency can be involved in infectious diseases, immunosuppression, organ failure, glandular problems, radiation illness, nutritional deficiency, poisoning, or other environmental or external insults.

The term “hormones” relates to a class of signaling molecules produced by glands, wherein signaling usually includes the following steps: (i) synthesis of a hormone in a particular tissue; (ii) storage and secretion; (iii) transport of the hormone to its target; (iv) binding of the hormone by a receptor; (v) relay and amplification of the signal; and (vi) breakdown of the hormone. Hormones differ from cytokines in that (1) hormones usually act in less variable concentrations and (2) generally are made by specific kinds of cells. In some embodiments, a “hormone” is a peptide or protein hormone, such as insulin, vasopressin, prolactin, adrenocorticotropic hormone (ACTH), thyroid hormone, growth hormones (such as human grown hormone or bovine somatotropin), oxytocin, atrial-natriuretic peptide (ANP), glucagon, somatostatin, cholecystokinin, gastrin, and leptins.

The term “adhesion molecules” relates to proteins which are located on the surface of a cell and which are involved in binding of the cell with other cells or with the extracellular matrix (ECM). Adhesion molecules are typically transmembrane receptors and can be classified as calcium-independent (e.g., integrins, immunoglobulin superfamily, lymphocyte homing receptors) and calcium-dependent (cadherins and selectins). Particular examples of adhesion molecules are integrins, lymphocyte homing receptors, selectins (e.g., P-selectin), and addressins.

Integrins are also involved in signal transduction. In particular, upon ligand binding, integrins modulate cell signaling pathways, e.g., pathways of transmembrane protein kinases such as receptor tyrosine kinases (RTK). Such regulation can lead to cellular growth, division, survival, or differentiation or to apoptosis. Particular examples of integrins include: α₁β₁, α₂β₁, α₃β₁, α₄β₁, α₅β₁, α₆β₁, α₇β₁, α_{L}β₂, α_{M}β₂, α_{IIb} β₃, α_{V}β₁, α_{V}β₃, α_{V}β₅, α_{V}β₆, α_{V}β₈, and α₆β₄.

The term “immunoglobulins” or “immunoglobulin superfamily” refers to molecules which are involved in the recognition, binding, and/or adhesion processes of cells. Molecules belonging to this superfamily share the feature that they contain a region known as immunoglobulin domain or fold. Members of the immunoglobulin superfamily include antibodies (e.g., IgG), T cell receptors (TCRs), major histocompatibility complex (MHC) molecules, co-receptors (e.g., CD4, CD8, CD19), antigen receptor accessory molecules (e.g., CD-3γ, CD3-δ, CD-3ε, CD79a, CD79b), co-stimulatory or inhibitory molecules (e.g., CD28, CD80, CD86), and other.

The term “immunologically active compound” relates to any compound altering an immune response, preferably by inducing and/or suppressing maturation of immune cells, inducing and/or suppressing cytokine biosynthesis, and/or altering humoral immunity by stimulating antibody production by B cells. Immunologically active compounds possess potent immunostimulating activity including, but not limited to, antiviral and antitumor activity, and can also down-regulate other aspects of the immune response, for example shifting the immune response away from a TH2 immune response, which is useful for treating a wide range of TH2 mediated diseases. Immunologically active compounds can be useful as vaccine adjuvants. Particular examples of immunologically active compounds include interleukins, colony stimulating factor (CSF), granulocyte colony stimulating factor (G-CSF), granulocyte-macrophage colony stimulating factor (GM-CSF), erythropoietin, tumor necrosis factor (TNF), interferons, integrins, addressins, selectins, homing receptors, T cell receptors, chimeric antigen receptors (CARs), immunoglobulins, and antigens, in particular tumor-associated antigens, pathogen-associated antigens (such as bacterial, parasitic, or viral antigens), allergens, autoantigens, hormones (insulin, thyroid hormone, catecholamines, gonadotrophines, trophic hormones, prolactin, oxytocin, dopamine, bovine somatotropin, leptins and the like), growth hormones (e.g., human grown hormone), growth factors (e.g., epidermal growth factor, nerve growth factor, insulin-like growth factor and the like), growth factor receptors, enzymes (tissue plasminogen activator, streptokinase, cholesterol biosynthestic or degradative, steriodogenic enzymes, kinases, phosphodiesterases, methylases, de-methylases, dehydrogenases, cellulases, proteases, lipases, phospholipases, aromatases, cytochromes, adenylate or guanylaste cyclases, neuramidases, lysosomal enzymes and the like), receptors (steroid hormone receptors, peptide receptors), binding proteins (growth hormone or growth factor binding proteins and the like), transcription and translation factors, tumor growth suppressing proteins (e.g., proteins which inhibit angiogenesis), structural proteins (such as collagen, fibroin, fibrinogen, elastin, tubulin, actin, and myosin), blood proteins (thrombin, serum albumin, Factor VII, Factor VIII, insulin, Factor IX, Factor X, tissue plasminogen activator, protein C, von Wilebrand factor, antithrombin III, glucocerebrosidase, erythropoietin granulocyte colony stimulating factor (GCSF) or modified Factor VIII, anticoagulants and the like. A preferred immunologically active compound is a vaccine antigen, i.e., an antigen whose inoculation into a subject induces an immune response.

In some embodiments, nucleic acid (such as DNA or RNA, such as mRNA) used in the present disclosure comprises a nucleic acid sequence encoding a peptide or polypeptide comprising an epitope for inducing an immune response against an antigen in a subject. The “peptide or polypeptide comprising an epitope for inducing an immune response against an antigen in a subject” is also designated herein as “vaccine antigen”, “peptide and protein antigen” or simply “antigen”.

In some embodiments, the RNA encoding vaccine antigen is a single-stranded, 5′ capped mRNA that is translated into the respective protein upon entering cells of a subject being administered the RNA, e.g., antigen-presenting cells (APCs). Preferably, the RNA (i) contains structural elements optimized for maximal efficacy of the RNA with respect to stability and translational efficiency (5′ cap, 5′ UTR, 3′ UTR, poly(A) sequence); (ii) is modified for optimized efficacy of the RNA (e.g., increased translation efficacy, decreased immunogenicity, and/or decreased cytotoxicity) (e.g., by replacing (partially or completely, preferably completely) naturally occurring nucleosides (in particular cytidine) with synthetic nucleosides (e.g., modified nucleosides selected from the group consisting of pseudouridine (ψ). N1-methyl-pseudouridine (mlv), and 5-methyl-uridine); and/or codon-optimization), or (iii) both (i) and (ii).

In some embodiments, beta-S-ARCA (D1) is utilized as specific capping structure at the 5′-end of the RNA. In some embodiments, the 5′-UTR comprises the nucleotide sequence of SEQ ID NO: 6, or a nucleotide sequence having at least 99%, 98%, 97%, 96%, 95%, 90%, 85%, or 80% identity to the nucleotide sequence of SEQ ID NO: 6. In some embodiments, the 3′-UTR comprises the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7.

In some embodiments, the poly(A) sequence is 110 nucleotides in length and consists of a stretch of 30 adenosine residues, followed by a 10 nucleotide linker sequence and another 70 adenosine residues. This poly(A) sequence was designed to enhance RNA stability and translational efficiency in dendritic cells. In some embodiments, the poly(A) sequence comprises the nucleotide sequence of SEQ ID NO: 8, or a nucleotide sequence having at least 99%, 98%, 97%, 96%, 95%, 90%, 85%, or 80% identity to the nucleotide sequence of SEQ ID NO: 8.

In some embodiments, the RNA comprises a modified nucleoside in place of uridine. In some embodiments, the modified nucleoside replacing (partially or completely, preferably completely) uridine is selected from the group consisting of pseudouridine (ψ), N1-methyl-pseudouridine (m1ψ), and 5-methyl-uridine. In some embodiments, the RNA encoding the vaccine antigen has a coding sequence (a) which is codon-optimized, (b) the G/C content of which is increased compared to the wild type coding sequence, or (c) both (a) and (b).

In some embodiments, the nucleic acid (such as DNA or RNA) encoding the vaccine antigen is expressed in cells of the subject to provide the vaccine antigen. In some embodiments, expression of the vaccine antigen is at the cell surface. In some embodiments, the vaccine antigen is presented in the context of MHC. In some embodiments, the nucleic acid (such as DNA or RNA) encoding the vaccine antigen is transiently expressed in cells of the subject. In some embodiments, the nucleic acid (such as DNA or RNA) encoding the vaccine antigen is administered systemically. In some embodiments, after systemic administration of the nucleic acid (such as DNA or RNA) encoding the vaccine antigen, expression of the nucleic acid encoding the vaccine antigen in spleen occurs. In some embodiments, after systemic administration of the nucleic acid encoding the vaccine antigen, expression of the nucleic acid encoding the vaccine antigen in antigen presenting cells, preferably professional antigen presenting cells occurs. In some embodiments, the antigen presenting cells are selected from the group consisting of dendritic cells, macrophages and B cells. In some embodiments, after systemic administration of the nucleic acid encoding the vaccine antigen, no or essentially no expression of the nucleic acid encoding the vaccine antigen in lung and/or liver occurs. In some embodiments, after systemic administration of the nucleic acid encoding the vaccine antigen, expression of the nucleic acid encoding the vaccine antigen in spleen is at least 5-fold the amount of expression in lung.

The vaccine antigen comprises an epitope for inducing an immune response against an antigen in a subject. Accordingly, the vaccine antigen comprises an antigenic sequence for inducing an immune response against an antigen in a subject. Such antigenic sequence may correspond to a target antigen or disease-associated antigen, e.g., a protein of an infectious agent (e.g., viral or bacterial antigen) or tumor antigen, or may correspond to an immunogenic variant thereof, or an immunogenic fragment of the target antigen or disease-associated antigen or the immunogenic variant thereof. Thus, the antigenic sequence may comprise at least an epitope of a target antigen or disease-associated antigen or an immunogenic variant thereof.

The antigenic sequences, e.g., epitopes, suitable for use according to the disclosure typically may be derived from a target antigen, i.e. the antigen against which an immune response is to be elicited. For example, the antigenic sequences contained within the vaccine antigen may be a target antigen or a fragment or variant of a target antigen.

The antigenic sequence or a procession product thereof, e.g., a fragment thereof, may bind to the antigen receptor such as TCR or CAR carried by immune effector cells. In some embodiments, the antigenic sequence is selected from the group consisting of the antigen expressed by a target cell to which the immune effector cells are targeted or a fragment thereof, or a variant of the antigenic sequence or the fragment.

A vaccine antigen which is provided to a subject according to the present disclosure by administering nucleic acid (such as DNA or RNA) encoding the vaccine antigen, preferably results in the induction of an immune response, e.g., in the stimulation, priming and/or expansion of immune effector cells, in the subject being provided the vaccine antigen. Said immune response, e.g., stimulated, primed and/or expanded immune effector cells, is preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In some embodiments, such fragment or variant is immunologically equivalent to the disease-associated antigen.

In the context of the present disclosure, the term “fragment of an antigen” or “variant of an antigen” means an agent which results in the induction of an immune response, e.g., in the stimulation, priming and/or expansion of immune effector cells, which immune response, e.g., stimulated, primed and/or expanded immune effector cells, targets the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of inducing an immune response against, e.g., stimulating, priming and/or expanding immune effector cells carrying an antigen receptor binding to, the antigen or cells expressing the antigen. It is preferred that the vaccine antigen (similar to the disease-associated antigen) provides the relevant epitope for binding by the antigen receptor present on the immune effector cells. In some embodiments, the vaccine antigen or a fragment thereof (similar to the disease-associated antigen) is expressed on the surface of a cell such as an antigen-presenting cell (optionally in the context of MHC) so as to provide the relevant epitope for binding by immune effector cells. The vaccine antigen may be a recombinant antigen.

In some embodiments of all aspects of the present disclosure, the nucleic acid (such as DNA or RNA) encoding the vaccine antigen is expressed in cells of a subject to provide the antigen or a procession product thereof for binding by the antigen receptor expressed by immune effector cells, said binding resulting in stimulation, priming and/or expansion of the immune effector cells.

In some embodiments, an antigen is presented or present on the surface of cells of the immune system such as antigen presenting cells like dendritic cells or macrophages. An antigen or a procession product thereof such as a T cell epitope is in some embodiments bound by an antigen receptor. Accordingly, an antigen or a procession product thereof may react specifically with immune effector cells such as T-lymphocytes (T cells).

In some embodiments, an antigen is a molecule which, optionally after processing, induces an immune reaction, which may be specific for the antigen (including cells expressing the antigen). In some embodiments, an antigen is a disease-associated antigen, such as a tumor antigen, a viral antigen, or a bacterial antigen, or an epitope derived from such antigen.

According to some embodiments, an amino acid sequence enhancing antigen processing and/or presentation is fused, either directly or through a linker, to an antigenic peptide or polypeptide (antigenic sequence). Accordingly, in some embodiments, the nucleic acid (such as DNA or RNA) described herein comprises at least one coding region encoding an antigenic peptide or polypeptide and an amino acid sequence enhancing antigen processing and/or presentation.

In some embodiments, antigen for vaccination which may be administered in the form of nucleic acid (such as DNA or RNA) coding therefor comprises a naturally occurring antigen or a fragment such as an epitope thereof.

Such amino acid sequences enhancing antigen processing and/or presentation are preferably located at the C-terminus of the antigenic peptide or polypeptide (and optionally at the C-terminus of an amino acid sequence which breaks immunological tolerance), without being limited thereto. Amino acid sequences enhancing antigen processing and/or presentation as defined herein preferably improve antigen processing and presentation. In some embodiments, the amino acid sequence enhancing antigen processing and/or presentation as defined herein includes, without being limited thereto, sequences derived from the human MHC class I complex (HLA-B51, haplotype A2, B27/B51, Cw2/Cw3), in particular a sequence comprising the amino acid sequence of SEQ ID NO: 2 or a functional variant thereof.

In some embodiments, an amino acid sequence enhancing antigen processing and/or presentation comprises the amino acid sequence of SEQ ID NO: 2, an amino acid sequence having at least 99%, 98%, 97%, 96%, 95%, 90%, 85%, or 80% identity to the amino acid sequence of SEQ ID NO: 2, or a functional fragment of the amino acid sequence of SEQ ID NO: 2, or the amino acid sequence having at least 99%, 98%, 97%, 96%, 95%, 90%, 85%, or 80% identity to the amino acid sequence of SEQ ID NO: 2. In some embodiments, an amino acid sequence enhancing antigen processing and/or presentation comprises the amino acid sequence of SEQ ID NO: 2.

Accordingly, in some embodiments, the nucleic acid (such as DNA or RNA) described herein comprises at least one coding region encoding an antigenic peptide or polypeptide and an amino acid sequence enhancing antigen processing and/or presentation, said amino acid sequence enhancing antigen processing and/or presentation preferably being fused to the antigenic peptide or polypeptide, more preferably to the C-terminus of the antigenic peptide or polypeptide as described herein.

Furthermore, a secretory sequence, e.g., a sequence comprising the amino acid sequence of SEQ ID NO: 1, may be fused to the N-terminus of the antigenic peptide or polypeptide.

Amino acid sequences derived from tetanus toxoid of Clostridium tetani may be employed to overcome self-tolerance mechanisms in order to efficiently mount an immune response to self-antigens by providing T-cell help during priming.

It is known that tetanus toxoid heavy chain includes epitopes that can bind promiscuously to MHC class II alleles and induce CD4⁺ memory T cells in almost all tetanus vaccinated individuals. In addition, the combination of tetanus toxoid (TT) helper epitopes with tumor-associated antigens is known to improve the immune stimulation compared to application of tumor-associated antigen alone by providing CD4⁺-mediated T-cell help during priming. To reduce the risk of stimulating CD8⁺ T cells with the tetanus sequences which might compete with the intended induction of tumor antigen-specific T-cell response, not the whole fragment C of tetanus toxoid is used as it is known to contain CD8⁺ T-cell epitopes. Two peptide sequences containing promiscuously binding helper epitopes were selected alternatively to ensure binding to as many MHC class II alleles as possible. Based on the data of the ex vivo studies the well-known epitopes p2 (QYIKANSKFIGITEL; TT₈₃₀₋₈₄₄; SEQ ID NO: 9) and p16 (MTNSVDDALINSTKIYSYFPSVISKVNQGAQG; TT₅₇₈₋₆₀₉; SEQ ID NO: 10) were selected. The p2 epitope was already used for peptide vaccination in clinical trials to boost anti-melanoma activity.

Non-clinical data showed that RNA vaccines encoding both a tumor antigen plus promiscuously binding tetanus toxoid sequences lead to enhanced CD8⁺ T-cell responses directed against the tumor antigen and improved break of tolerance. Immunomonitoring data from patients vaccinated with vaccines including those sequences fused in frame with the tumor antigen-specific sequences reveal that the tetanus sequences chosen are able to induce tetanus-specific T-cell responses in almost all patients.

According to some embodiments, an amino acid sequence which breaks immunological tolerance is fused, either directly or through a linker, e.g., a linker having the amino acid sequence according to SEQ ID NO: 4, to the antigenic peptide or polypeptide.

Such amino acid sequences which break immunological tolerance are preferably located at the C-terminus of the antigenic peptide or polypeptide (and optionally at the N-terminus of the amino acid sequence enhancing antigen processing and/or presentation, wherein the amino acid sequence which breaks immunological tolerance and the amino acid sequence enhancing antigen processing and/or presentation may be fused either directly or through a linker, e.g., a linker having the amino acid sequence according to SEQ ID NO: 5), without being limited thereto. Amino acid sequences which break immunological tolerance as defined herein preferably improve T cell responses. In some embodiments, the amino acid sequence which breaks immunological tolerance as defined herein includes, without being limited thereto, sequences derived from tetanus toxoid-derived helper sequences p2 and p16 (P2P16), in particular a sequence comprising the amino acid sequence of SEQ ID NO: 3 or a functional variant thereof.

In some embodiments, an amino acid sequence which breaks immunological tolerance comprises the amino acid sequence of SEQ ID NO: 3, an amino acid sequence having at least 99%, 98%, 97%, 96%, 95%, 90%, 85%, or 80% identity to the amino acid sequence of SEQ ID NO: 3, or a functional fragment of the amino acid sequence of SEQ ID NO: 3, or the amino acid sequence having at least 99%, 98%, 97%, 96%, 95%, 90%, 85%, or 80% identity to the amino acid sequence of SEQ ID NO: 3. In some embodiments, an amino acid sequence which breaks immunological tolerance comprises the amino acid sequence of SEQ ID NO: 3.

In the following, embodiments of vaccine RNAs are described, wherein certain terms used when describing elements thereof have the following meanings:
hAg-Kozak: 5′-UTR sequence of the human alpha-globin mRNA with an optimized ‘Kozak sequence’ to increase translational efficiency.
see/MITD: Fusion-protein tags derived from the sequence encoding the human MHC class I complex (HLA-B51, haplotype A2, B27/B51, Cw2/Cw3), which have been shown to improve antigen processing and presentation. Sec corresponds to the 78 bp fragment coding for the secretory signal peptide, which guides translocation of the nascent polypeptide chain into the endoplasmatic reticulum. MITD corresponds to the transmembrane and cytoplasmic domain of the MHC class I molecule, also called MHC class I trafficking domain.
Antigen: Sequences encoding the respective vaccine antigen/epitope.
Glycine-serine linker (GS): Sequences coding for short linker peptides predominantly consisting of the amino acids glycine (G) and serine(S), as commonly used for fusion proteins.
P2P16: Sequence coding for tetanus toxoid-derived helper epitopes to break immunological tolerance.
FI element: The 3′-UTR is a combination of two sequence elements derived from the “amino terminal enhancer of split” (AES) mRNA (called F) and the mitochondrial encoded 12S ribosomal RNA (called I). These were identified by an ex vivo selection process for sequences that confer RNA stability and augment total protein expression.
A30L₇₀: A poly(A)-tail measuring 110 nucleotides in length, consisting of a stretch of 30 adenosine residues, followed by a 10 nucleotide linker sequence and another 70 adenosine residues designed to enhance RNA stability and translational efficiency in dendritic cells.

In some embodiments, vaccine RNA described herein has one of the following structures:

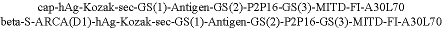

In some embodiments, vaccine antigen described herein has the structure:

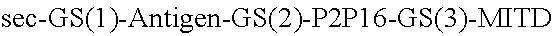

In some embodiments, hAg-Kozak comprises the nucleotide sequence of SEQ ID NO: 6. In some embodiments, see comprises the amino acid sequence of SEQ ID NO: 1. In some embodiments, P2P16 comprises the amino acid sequence of SEQ ID NO: 3. In some embodiments, MITD comprises the the amino acid sequence of SEQ ID NO: 2. In some embodiments, GS (1) comprises the amino acid sequence of SEQ ID NO: 4. In some embodiments, GS (2) comprises the amino acid sequence of SEQ ID NO: 4. In some embodiments, GS (3) comprises the amino acid sequence of SEQ ID NO: 5. In some embodiments, FI comprises the nucleotide sequence of SEQ ID NO: 7. In some embodiments, A30L₇₀ comprises the nucleotide sequence of SEQ ID NO: 8.

In some embodiments, the sequence encoding the vaccine antigen/epitope comprises a modified nucleoside replacing (partially or completely, preferably completely) uridine, wherein the modified nucleoside is selected from the group consisting of pseudouridine (ψ), N1-methyl-pseudouridine (m1ψ), and 5-methyl-uridine.

In some embodiments, the sequence encoding the vaccine antigen/epitope is codon-optimized.

In some embodiments, the G/C content of the sequence encoding the vaccine antigen/epitope is increased compared to the wild type coding sequence.

In some embodiments, an antigen receptor is an antibody or B cell receptor which binds to an epitope in an antigen. In some embodiments, an antibody or B cell receptor binds to native epitopes of an antigen.

The term “autoantigen” or “self-antigen” refers to an antigen which originates from within the body of a subject (i.e., the autoantigen can also be called “autologous antigen”) and which produces an abnormally vigorous immune response against this normal part of the body. Such vigorous immune reactions against autoantigens may be the cause of “autoimmune diseases”.

The term “allergen” refers to a kind of antigen which originates from outside the body of a subject (i.e., the allergen can also be called “heterologous antigen”) and which produces an abnormally vigorous immune response in which the immune system of the subject fights off a perceived threat that would otherwise be harmless to the subject. “Allergies” are the diseases caused by such vigorous immune reactions against allergens. An allergen usually is an antigen which is able to stimulate a type-I hypersensitivity reaction in atopic individuals through immunoglobulin E (IgE) responses. Particular examples of allergens include allergens derived from peanut proteins (e.g., Ara h 2.02), ovalbumin, grass pollen proteins (e.g., Ph1 p 5), and proteins of dust mites (e.g., Der p 2).

The term “growth factors” refers to molecules which are able to stimulate cellular growth, proliferation, healing, and/or cellular differentiation. Typically, growth factors act as signaling molecules between cells. The term “growth factors” include particular cytokines and hormones which bind to specific receptors on the surface of their target cells. Examples of growth factors include bone morphogenetic proteins (BMPs), fibroblast growth factors (FGFs), vascular endothelial growth factors (VEGFs), such as VEGFA, epidermal growth factor (EGF), insulin-like growth factor, ephrins, macrophage colony-stimulating factor, granulocyte colony-stimulating factor, granulocyte macrophage colony-stimulating factor, neuregulins, neurotrophins (e.g., brain-derived neurotrophic factor (BDNF), nerve growth factor (NGF)), placental growth factor (PGF), platelet-derived growth factor (PDGF), renalase (RNLS) (anti-apoptotic survival factor), T-cell growth factor (TCGF), thrombopoietin (TPO), transforming growth factors (transforming growth factor alpha (TGF-α), transforming growth factor beta (TGF-β)), and tumor necrosis factor-alpha (TNF-α). In some embodiments, a “growth factor” is a peptide or protein growth factor.

The term “protease inhibitors” refers to molecules, in particular peptides or proteins, which inhibit the function of proteases. Protease inhibitors can be classified by the protease which is inhibited (e.g., aspartic protease inhibitors) or by their mechanism of action (e.g., suicide inhibitors, such as serpins).

Particular examples of protease inhibitors include serpins, such as alpha 1-antitrypsin, aprotinin, and bestatin.

The term “enzymes” refers to macromolecular biological catalysts which accelerate chemical reactions. Like any catalyst, enzymes are not consumed in the reaction they catalyze and do not alter the equilibrium of said reaction. Unlike many other catalysts, enzymes are much more specific. In some embodiments, an enzyme is essential for homeostasis of a subject, e.g., any malfunction (in particular, decreased activity which may be caused by any of mutation, deletion or decreased production) of the enzyme results in a disease. Examples of enzymes include herpes simplex virus type 1 thymidine kinase (HSV1-TK), hexosaminidase, phenylalanine hydroxylase, pseudocholinesterase, and lactase.

The term “receptors” refers to protein molecules which receive signals (in particular chemical signals called ligands) from outside a cell. The binding of a signal (e.g., ligand) to a receptor causes some kind of response of the cell, e.g., the intracellular activation of a kinase. Receptors include transmembrane receptors (such as ion channel-linked (ionotropic) receptors, G protein-linked (metabotropic) receptors, and enzyme-linked receptors) and intracellular receptors (such as cytoplasmic receptors and nuclear receptors). Particular examples of receptors include steroid hormone receptors, growth factor receptors, and peptide receptors (i.e., receptors whose ligands are peptides), such as P-selectin glycoprotein ligand-1 (PSGL-1). The term “growth factor receptors” refers to receptors which bind to growth factors.

The term “apoptosis regulators” refers to molecules, in particular peptides or proteins, which modulate apoptosis, i.e., which either activate or inhibit apoptosis. Apoptosis regulators can be grouped into two broad classes: those which modulate mitochondrial function and those which regulate caspases. The first class includes proteins (e.g., BCL-2, BCL-xL) which act to preserve mitochondrial integrity by preventing loss of mitochondrial membrane potential and/or release of pro-apoptotic proteins such as cytochrome C into the cytosol. Also to this first class belong proapoptotic proteins (e.g., BAX, BAK, BIM) which promote release of cytochrome C. The second class includes proteins such as the inhibitors of apoptosis proteins (e.g., XIAP) or FLIP which block the activation of caspases.

The term “transcription factors” relates to proteins which regulate the rate of transcription of genetic information from DNA to messenger RNA, in particular by binding to a specific DNA sequence. Transcription factors may regulate cell division, cell growth, and cell death throughout life; cell migration and organization during embryonic development; and/or in response to signals from outside the cell, such as a hormone. Transcription factors contain at least one DNA-binding domain which binds to a specific DNA sequence, usually adjacent to the genes which are regulated by the transcription factors. Particular examples of transcription factors include MECP2, FOXP2, FOXP3, the STAT protein family, and the HOX protein family.

The term “tumor suppressor proteins” relates to molecules, in particular peptides or proteins, which protect a cell from one step on the path to cancer. Tumor-suppressor proteins (usually encoded by corresponding tumor-suppressor genes) exhibit a weakening or repressive effect on the regulation of the cell cycle and/or promote apoptosis. Their functions may be one or more of the following: repression of genes essential for the continuing of the cell cycle; coupling the cell cycle to DNA damage (as long as damaged DNA is present in a cell, no cell division should take place); initiation of apoptosis, if the damaged DNA cannot be repaired; metastasis suppression (e.g., preventing tumor cells from dispersing, blocking loss of contact inhibition, and inhibiting metastasis); and DNA repair. Particular examples of tumor-suppressor proteins include p53, phosphatase and tensin homolog (PTEN), SWI/SNF (SWItch/Sucrose Non-Fermentable), von Hippel-Lindau tumor suppressor (pVHL), adenomatous polyposis coli (APC), CD95, suppression of tumorigenicity 5 (ST5), suppression of tumorigenicity 5 (ST5), suppression of tumorigenicity 14 (ST14), and Yippee-like 3 (YPEL3).

The term “structural proteins” refers to proteins which confer stiffness and rigidity to otherwise-fluid biological components. Structural proteins are mostly fibrous (such as collagen and elastin) but may also be globular (such as actin and tubulin). Usually, globular proteins are soluble as monomers, but polymerize to form long, fibers which, for example, may make up the cytoskeleton. Other structural proteins are motor proteins (such as myosin, kinesin, and dynein) which are capable of generating mechanical forces, and surfactant proteins. Particular examples of structural proteins include collagen, surfactant protein A, surfactant protein B, surfactant protein C, surfactant protein D, elastin, tubulin, actin, and myosin.

The term “reprogramming factors” or “reprogramming transcription factors” relates to molecules, in particular peptides or proteins, which, when expressed in somatic cells optionally together with further agents such as further reprogramming factors, lead to reprogramming or de-differentiation of said somatic cells to cells having stem cell characteristics, in particular pluripotency. Particular examples of reprogramming factors include OCT4, SOX2, c-MYC, KLF4, LIN28, and NANOG.

The term “genomic engineering proteins” relates to proteins which are able to insert, delete or replace DNA in the genome of a subject. Particular examples of genomic engineering proteins include meganucleases, zinc finger nucleases (ZFNs), transcription activator-like effector nucleases (TALENs), and clustered regularly spaced short palindromic repeat-CRISPR-associated protein 9 (CRISPR-Cas9).

The term “blood proteins” relates to peptides or proteins which are present in blood plasma of a subject, in particular blood plasma of a healthy subject. Blood proteins have diverse functions such as transport (e.g., albumin, transferrin), enzymatic activity (e.g., thrombin or ceruloplasmin), blood clotting (e.g., fibrinogen), defense against pathogens (e.g., complement components and immunoglobulins), protease inhibitors (e.g., alpha 1-antitrypsin), etc. Particular examples of blood proteins include thrombin, serum albumin, Factor VII, Factor VIII, insulin, Factor IX, Factor X, tissue plasminogen activator, protein C, von Willebrand factor, antithrombin III, glucocerebrosidase, erythropoietin, granulocyte colony stimulating factor (G-CSF), modified Factor VIII, and anticoagulants.

Thus, in some embodiments, the pharmaceutically active peptide or protein is (i) a cytokine, preferably selected from the group consisting of erythropoietin (EPO), interleukin 4 (IL-2), and interleukin 10 (IL-11), more preferably EPO; (ii) an adhesion molecule, in particular an integrin; (iii) an immunoglobulin, in particular an antibody; (iv) an immunologically active compound, in particular an antigen, such as a viral or bacterial antigen, e.g., an antigen of SARS-COV-2; (v) a hormone, in particular vasopressin, insulin or growth hormone; (vi) a growth factor, in particular VEGFA; (vii) a protease inhibitor, in particular alpha 1-antitrypsin; (viii) an enzyme, preferably selected from the group consisting of herpes simplex virus type 1 thymidine kinase (HSV1-TK), hexosaminidase, phenylalanine hydroxylase, pseudocholinesterase, pancreatic enzymes, and lactase; (ix) a receptor, in particular growth factor receptors; (x) an apoptosis regulator, in particular BAX; (xi) a transcription factor, in particular FOXP3; (xii) a tumor suppressor protein, in particular p53; (xiii) a structural protein, in particular surfactant protein B; (xiv) a reprogramming factor, e.g., selected from the group consisting of OCT4, SOX2, c-MYC. KLF4, LIN28 and NANOG; (xv) a genomic engineering protein, in particular clustered regularly spaced short palindromic repeat-CRISPR-associated protein 9 (CRISPR-Cas9); and (xvi) a blood protein, in particular fibrinogen.

In some embodiments, a pharmaceutically active peptide or protein comprises one or more antigens or one or more epitopes, i.e., administration of the peptide or protein to a subject elicits an immune response against the one or more antigens or one or more epitopes in a subject which may be therapeutic or partially or fully protective.

In certain embodiments, the nucleiac acid (such as DNA or RNA, preferably mRNA) encodes at least one epitope, e.g., at least two epitopes, at least three epitopes, at least four epitopes, at least five epitopes, at least six epitopes, at least seven epitopes, at least eight epitopes, at least nine epitopes, or at least ten epitopes.

In certain embodiments, the target antigen is a tumor antigen and the antigenic sequence (e.g., an epitop) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system. A neo-antigen or neo-epitope may result from one or more cancer-specific mutations in the genome of cancer cells resulting in amino acid changes. If the tumor antigen is a neo-antigen, the vaccine antigen preferably comprises an epitope or a fragment of said neo-antigen comprising one or more amino acid changes.

Examples of tumor antigens include, without limitation, p53, ART-4, BAGE, beta-catenin/m, Bcr-abL CAMEL, CAP-1, CASP-8, CDC27/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A10, MAGE-A11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY—BR-1, pl90 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. Nucleic acid (such as DNA or RNA) can be used to deliver patient-specific tumor epitopes to a patient. Dendritic cells (DCs) residing in the spleen represent antigen-presenting cells of particular interest for RNA expression of immunogenic epitopes or antigens such as tumor epitopes. The use of multiple epitopes has been shown to promote therapeutic efficacy in tumor vaccine compositions. Rapid sequencing of the tumor mutanome may provide multiple epitopes for individualized vaccines which can be encoded by nucleic acid (e.g., DNA or RNA, such as mRNA) described herein, e.g., as a single polypeptide wherein the epitopes are optionally separated by linkers. In certain embodiments of the present disclosure, the nucleic acid (e.g., DNA or RNA, such as mRNA) encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include nucleic acid (e.g., DNA or RNA, such as mRNA) that encodes at least five epitopes (termed a “pentatope”), nucleic acid (e.g., DNA or RNA, such as mRNA) that encodes at least ten epitopes (termed a “decatope”), and nucleic acid (e.g., DNA or RNA, such as mRNA) that encodes at least twenty epitopes (termed an “eicosatope”).

In certain embodiments, the epitope is derived from a pathogen-associated antigen, in particular from a viral antigen. In some embodiments, the epitope is derived from a SARS-COV-2 S protein, an immunogenic variant thereof, or an immunogenic fragment of the SARS-COV-2 S protein or the immunogenic variant thereof. Thus, in some embodiments, the nucleic acid (e.g., DNA or RNA, preferably mRNA) used in the present disclosure encodes an amino acid sequence comprising a SARS-CoV-2 S protein, an immunogenic variant thereof, or an immunogenic fragment of the SARS-COV-2 S protein or the immunogenic variant thereof.

In some embodiments, the nucleic acid (e.g., DNA or RNA, in particular, mRNA) described herein is a modified RNA, in particular a stabilized mRNA. In some embodiments, the RNA comprises a modified nucleoside in place of at least one uridine. In some embodiments, the RNA comprises a modified nucleoside in place of each uridine. In some embodiments, the modified nucleoside is independently selected from pseudouridine (ψ), N1-methyl-pseudouridine (m1ψ), and 5-methyl-uridine (m5U).

In some embodiments, the RNA (in particular, mRNA) described herein comprises a modified nucleoside in place of uridine.

In some embodiments, the modified nucleoside is selected from pseudouridine (ψ), N1-methyl-pseudouridine (mlv), and 5-methyl-uridine (m5U).

In some embodiments, the RNA (in particular, mRNA) described herein comprises a 5′ cap. In some embodiments, m₂^{7,3′-O}Gppp(m₁^{2′-O})ApG is utilized as specific capping structure at the 5′-end of the mRNA.

In some embodiments, the RNA (in particular, mRNA) encoding an antigen (in particular the vaccine RNA) comprises a 5′ UTR comprising the nucleotide sequence of SEQ ID NO: 6, or a nucleotide sequence having at least 99%, 98%, 97%, 96%, 95%, 90%, 85%, or 80% identity to the nucleotide sequence of SEQ ID NO: 6.

In some embodiments, the RNA (in particular, mRNA) encoding an antigen (in particular the vaccine RNA) comprises a 3′ UTR comprising the nucleotide sequence of SEQ ID NO: 7, or a nucleotide sequence having at least 99%, 98%, 97%, 96%, 95%, 90%, 85%, or 80% identity to the nucleotide sequence of SEQ ID NO: 7.

In some embodiments, the RNA (in particular, mRNA) encoding an antigen (in particular the vaccine RNA) comprises a poly-A sequence. In some embodiments, the poly-A sequence comprises at least 100 nucleotides. In some embodiments, the poly-A sequence comprises or consists of the nucleotide sequence of SEQ ID NO: 8.

In some embodiments, the nucleic acid (e.g., DNA or RNA, in particular, mRNA) described herein is formulated or is to be formulated as a liquid, a solid, or a combination thereof.

In some embodiments, the nucleic acid (e.g., DNA or RNA, in particular, mRNA) described herein is formulated or is to be formulated for injection.

In some embodiments, the nucleic acid (e.g., DNA or RNA, in particular, mRNA) described herein is formulated or is to be formulated for intramuscular administration.

In some embodiments, the nucleic acid (e.g., DNA or RNA, in particular, mRNA) described herein is formulated or is to be formulated as a composition, e.g., a pharmaceutical composition.

In some embodiments, the composition comprises (i) nucleic acid (e.g., DNA or RNA); (ii) a cationic or cationically ionizable lipid; and (iii) a conjugate of formula (II) (or of any one of the formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)).

In some embodiments, the composition comprises (i) nucleic acid (e.g., DNA or RNA); (ii) a cationic or cationically ionizable lipid selected from the group consisting of DODMA, DOTMA, DPL14, 3D-P-DMA, Formula (X), and Formula (XI), (e.g., any one of Formulas (XIIa), (XIIb), (XIIIa), (XIIIb), (XIV-1), (XIV-2), and (XIV-3)); (iii) a conjugate of formula (II) (or of any one of the formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); and (iv) one or more additional lipids. In some embodiments, the one or more additional lipids are selected from neutral lipids and combinations thereof. In some embodiments, the neutral lipids include phospholipids, steroid lipids, and combinations thereof. In some embodiments, the one or more additional lipids are a combination of a phospholipid and a steroid lipid.

In some embodiments, the composition comprises (i) nucleic acid (e.g., DNA or RNA); (ii) a cationic or cationically ionizable lipid selected from the group consisting of DODMA, DOTMA, DPL14, 3D-P-DMA, Formula (X), and Formula (XI), (e.g., any one of Formulas (XIIa), (XIIb), (XIIIa), (XIIIb), (XIV-1), (XIV-2), and (XIV-3)); (iii) a conjugate of formula (II) (or of any one of the formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); (iv) a phospholipid; and (v) cholesterol. In some embodiments, the phospholipid is DOPE.

In some embodiments, the composition comprises (i) nucleic acid (e.g., DNA or RNA); (ii) a cationic or cationically ionizable lipid selected from the group consisting of DODMA, DOTMA, DPL14, 3D-P-DMA, and Formula (XI), (e.g., any one of Formulas (XIIa), (XIIb), (XIIIa), (XIIIb), (XIV-1), (XIV-2), and (XIV-3)); (iii) a conjugate of formula (II) (or of any one of the formulas (IIa), (IIa), (IIb), (IIb), (IIc), (IId), (IIc), (IIf), (IIg), (IIh), (IIi′), (IIj), (III), (III), (IIIa), (IIIa), (IIIb), (IIIb), (IV), (IVa), (IVb), (IVc), (IVd′), (IVc′), (IVf), (IVg′), (IVh), (V), (V′), (VI), (IV), (VI), (VIa), (VIa), (VII), (VII′), (VIIa), (VIIa), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIc), (IVc), (XV), (XV″), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); (iv) a phospholipid; and (v) cholesterol. In some embodiments, the phospholipid is DOPE.

In some embodiments, the composition comprises (i) nucleic acid (e.g., DNA or RNA); (ii) DODMA; (iii) a conjugate of formula (II) (or of any one of the formulas (IIa), (IIa′), (IIb), (IIb), (IIc), (IId), (IIe′), (IIf), (IIg), (IIh), (IIi′), (IIj), (III), (III), (IIIa), (IIIa), (IIIb), (IIIb), (IV), (IV), (IVa), (IVb), (IVc), (IVd′), (IVe), (IVf), (IVg), (IVh), (V), (V′), (VI), (VI), (VIa), (VIa′), (VII), (VII), (VIIa), (VIIa′), (VIII), (VIII), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIe), (IVc), (XV), (XV″), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); (iv) a phospholipid; and (v) cholesterol. In some embodiments, the phospholipid is DOPE.

In some embodiments, the composition comprises (i) nucleic acid (e.g., DNA or RNA); (ii) DOTMA; (iii) a conjugate of formula (II) (or of any one of the formulas (IIa), (IIa), (IIb), (IIb), (IIc), (IId), (IIc′), (IIf), (IIg), (IIh), (IIi′), (IIj′), (III), (III), (IIIa), (IIIa′), (IIIb), (IIIb), (IV), (IV), (IVa), (IVb), (IVc), (IVd′), (IVe′), (IVf), (IVg), (IVh), (V), (V′), (VI), (VI), (VIa), (VIa′), (VII), (VII), (VIIa), (VIIa), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIc), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); (iv) a phospholipid; and (v) cholesterol. In some embodiments, the phospholipid is DOPE.

In some embodiments, the composition comprises (i) nucleic acid (e.g., DNA or RNA); (ii) DPL14; (iii) a conjugate of formula (II) (or of any one of the formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIc), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); (iv) a phospholipid; and (v) cholesterol. In some embodiments, the phospholipid is DOPE.

In some embodiments, the composition comprises (i) nucleic acid (e.g., DNA or RNA); (ii) 3D-P-DMA; (iii) a conjugate of formula (II) (or of any one of the formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); (iv) a phospholipid; and (v) cholesterol. In some embodiments, the phospholipid is DOPE.

In some embodiments, the composition comprises (i) nucleic acid (e.g., DNA or RNA); (ii) a cationic or cationically ionizable lipid of Formula (XI), (e.g., any one of Formulas (XIIa), (XIIb), (XIIIa), (XIIIb), (XIV-1), (XIV-2), and (XIV-3)); (iii) a conjugate of formula (II) (or of any one of the formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or of any one of the formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)); (iv) a phospholipid; and (v) cholesterol. In some embodiments, the phospholipid is DOPE.

In some embodiments, the nucleic acid is DNA.

In some embodiments, the nucleic acid is RNA, in particular mRNA or saRNA.

In some embodiments, the composition, in particular the pharmaceutical composition, is a vaccine.

In some embodiments, the composition, in particular the pharmaceutical composition, further comprises one or more pharmaceutically acceptable carriers, diluents and/or excipients.

In some embodiments, the nucleic acid (e.g., DNA or RNA) and/or the composition, in particular the pharmaceutical composition, is/are a component of a kit.

In some embodiments, the kit further comprises instructions for use of the nucleic acid (e.g., DNA or RNA) for inducing an immune response against coronavirus in a subject. In some embodiments, the coronavirus is a betacoronavirus. In some embodiments, the coronavirus is a sarbecovirus. In some embodiments, the coronavirus is SARS-COV-2.

In some embodiments, the kit further comprises instructions for use of the nucleic acid (e.g., DNA or RNA) for therapeutically or prophylactically treating a coronavirus infection in a subject. In some embodiments, the coronavirus is a betacoronavirus. In some embodiments, the coronavirus is a sarbecovirus. In some embodiments, the coronavirus is SARS-COV-2.

In some embodiments, the subject is a human.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject induces an immune reaction having a specificity of reacting with the reference amino acid sequence. Thus, in some embodiments, a molecule which is immunologically equivalent to an antigen exhibits the same or essentially the same properties and/or exerts the same or essentially the same effects regarding the stimulation, priming and/or expansion of T cells as the antigen to which the T cells are targeted.

In some embodiments, the RNA (preferably mRNA), e.g., RNA encoding vaccine antigen, used in the present disclosure is non-immunogenic. RNA encoding an immunostimulant may be administered according to the present disclosure to provide an adjuvant effect. The RNA encoding an immunostimulant may be standard RNA or non-immunogenic RNA.

The term “non-immunogenic RNA” (such as “non-immunogenic mRNA”) as used herein refers to RNA that does not induce a response by the immune system upon administration, e.g., to a mammal, or induces a weaker response than would have been induced by the same RNA that differs only in that it has not been subjected to the modifications and treatments that render the non-immunogenic RNA non-immunogenic, i.e., than would have been induced by standard RNA (stdRNA). In certain embodiments, non-immunogenic RNA, which is also termed modified RNA (modRNA) herein, is rendered non-immunogenic by incorporating modified nucleosides suppressing RNA-mediated activation of innate immune receptors into the RNA and/or limiting the amount of double-stranded RNA (dsRNA), e.g., by limiting the formation of double-stranded RNA (dsRNA), e.g., during in vitro transcription, and/or by removing double-stranded RNA (dsRNA), e.g., following in vitro transcription. In certain embodiments, non-immunogenic RNA is rendered non-immunogenic by incorporating modified nucleosides suppressing RNA-mediated activation of innate immune receptors into the RNA and/or removing double-stranded RNA (dsRNA), e.g., following in vitro transcription.

For rendering the non-immunogenic RNA (especially mRNA) non-immunogenic by the incorporation of modified nucleosides, any modified nucleoside may be used as long as it lowers or suppresses immunogenicity of the RNA. Particularly preferred are modified nucleosides that suppress RNA-mediated activation of innate immune receptors. In some embodiments, the modified nucleosides comprise a replacement of one or more uridines with a nucleoside comprising a modified nucleobase. In some embodiments, the modified nucleobase is a modified uracil. In some embodiments, the nucleoside comprising a modified nucleobase is selected from the group consisting of 3-methyl-uridine (m³U). 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (hoSU), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl 5-ester (mchm⁵U), 5-methoxycarbonylmethyl-uridine (mcm⁵U), methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm₅se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (τm⁵s²U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine. N1-methyl-pseudouridine. 3-(3-amino-3-carboxypropyl) uridine (acp3U). 1-methyl-3-(3-amino-3-carboxypropyl) pseudouridine (acp³ ψ). 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridinc (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino) uridine. In certain embodiments, the nucleoside comprising a modified nucleobase is pseudouridine (ψ), N1-methyl-pseudouridine (mlv) or 5-methyl-uridine (m⁵U), in particular N1-methyl-pseudouridine.

In some embodiments, the replacement of one or more uridines with a nucleoside comprising a modified nucleobase comprises a replacement of at least 1%, at least 2%, at least 3%, at least 4%, at least 5%, at least 10%, at least 25%, at least 50%, at least 75%, at least 90%, at least 95%, at least 96%, at least 97%, at least 98%, at least 99% or 100% of the uridines.

During synthesis of RNA (preferably mRNA) by in vitro transcription (IVT) using T7 RNA polymerase significant amounts of aberrant products, including double-stranded RNA (dsRNA) are produced due to unconventional activity of the enzyme. dsRNA induces inflammatory cytokines and activates effector enzymes leading to protein synthesis inhibition. Formation of dsRNA can be limited during synthesis of mRNA by in vitro transcription (IVT), for example, by limiting the amount of uridine triphosphate (UTP) during synthesis. Optionally, UTP may be added once or several times during synthesis of mRNA. Also, dsRNA can be removed from RNA such as IVT RNA, for example, by ion-pair reversed phase HPLC using a non-porous or porous C-18 polystyrene-divinylbenzene (PS-DVB) matrix. Alternatively, an enzymatic based method using E. coli RNaseIII that specifically hydrolyzes dsRNA but not ssRNA, thereby eliminating dsRNA contaminants from IVT RNA preparations can be used. Furthermore, dsRNA can be separated from ssRNA by using a cellulose material. In some embodiments, an RNA preparation is contacted with a cellulose material and the ssRNA is separated from the cellulose material under conditions which allow binding of dsRNA to the cellulose material and do not allow binding of ssRNA to the cellulose material. Suitable methods for providing ssRNA are disclosed, for example, in WO 2017/182524.

As the term is used herein, “remove” or “removal” refers to the characteristic of a population of first substances, such as non-immunogenic RNA, being separated from the proximity of a population of second substances, such as dsRNA, wherein the population of first substances is not necessarily devoid of the second substance, and the population of second substances is not necessarily devoid of the first substance. However, a population of first substances characterized by the removal of a population of second substances has a measurably lower content of second substances as compared to the non-separated mixture of first and second substances.

In some embodiments, the amount of double-stranded RNA (dsRNA) is limited, e.g., dsRNA (especially dsmRNA) is removed from non-immunogenic RNA, such that less than 10%, less than 5%, less than 4%, less than 3%, less than 2%, less than 1%, less than 0.5%, less than 0.3%, less than 0.1%, less than 0.05%, less than 0.03%, less than 0.01%, less than 0.005%, less than 0.004%, less than 0.003%, less than 0.002%, less than 0.001%, or less than 0.0005% of the RNA in the non-immunogenic RNA composition is dsRNA. In some embodiments, the non-immunogenic RNA (especially mRNA) is free or essentially free of dsRNA. In some embodiments, the non-immunogenic RNA (especially mRNA) composition comprises a purified preparation of single-stranded nucleoside modified RNA. In some embodiments, the non-immunogenic RNA (especially mRNA) composition comprises single-stranded nucleoside modified RNA (especially mRNA) and is substantially free of double stranded RNA (dsRNA). In some embodiments, the non-immunogenic RNA (especially mRNA) composition comprises at least 90%, at least 91%, at least 92%, at least 93%, at least 94%, at least 95%, at least 96%, at least 97%, at least 98%, at least 99%, at least 99.5%, at least 99.9%, at least 99.99%, at least 99.991%, at least 99.992%, at least 99.993%, at least 99.994%, at least 99.995%, at least 99.996%, at least 99.997%, or at least 99.998% single stranded nucleoside modified RNA, relative to all other nucleic acid molecules (DNA, dsRNA, etc.).

Various methods can be used to determine the amount of dsRNA. For example, a sample may be contacted with dsRNA-specific antibody and the amount of antibody binding to RNA may be taken as a measure for the amount of dsRNA in the sample. A sample containing a known amount of dsRNA may be used as a reference.

For example, RNA may be spotted onto a membrane, e.g., nylon blotting membrane. The membrane may be blocked, e.g., in TBS-T buffer (20 mM TRIS pH 7.4, 137 mM NaCl, 0.1% (v/v) TWEEN-20) containing 5% (w/v) skim milk powder. For detection of dsRNA, the membrane may be incubated with dsRNA-specific antibody, e.g., dsRNA-specific mouse mAb (English & Scientific Consulting, Szirák, Hungary). After washing, e.g., with TBS-T, the membrane may be incubated with a secondary antibody, e.g., HRP-conjugated donkey anti-mouse IgG (Jackson ImmunoResearch, Cat #715-035-150), and the signal provided by the secondary antibody may be detected.

In some embodiments, the non-immunogenic RNA (especially mRNA) is translated in a cell more efficiently than standard RNA with the same sequence. In some embodiments, translation is enhanced by a factor of 2-fold relative to its unmodified counterpart. In some embodiments, translation is enhanced by a 3-fold factor. In some embodiments, translation is enhanced by a 4-fold factor. In some embodiments, translation is enhanced by a 5-fold factor. In some embodiments, translation is enhanced by a 6-fold factor. In some embodiments, translation is enhanced by a 7-fold factor. In some embodiments, translation is enhanced by an 8-fold factor. In some embodiments, translation is enhanced by a 9-fold factor. In some embodiments, translation is enhanced by a 10-fold factor. In some embodiments, translation is enhanced by a 15-fold factor. In some embodiments, translation is enhanced by a 20-fold factor. In some embodiments, translation is enhanced by a 50-fold factor. In some embodiments, translation is enhanced by a 100-fold factor. In some embodiments, translation is enhanced by a 200-fold factor. In some embodiments, translation is enhanced by a 500-fold factor. In some embodiments, translation is enhanced by a 1000-fold factor. In some embodiments, translation is enhanced by a 2000-fold factor. In some embodiments, the factor is 10-1000-fold. In some embodiments, the factor is 10-100-fold. In some embodiments, the factor is 10-200-fold. In some embodiments, the factor is 10-300-fold. In some embodiments, the factor is 10-500-fold. In some embodiments, the factor is 20-1000-fold. In some embodiments, the factor is 30-1000-fold. In some embodiments, the factor is 50-1000-fold. In some embodiments, the factor is 100-1000-fold. In some embodiments, the factor is 200-1000-fold. In some embodiments, translation is enhanced by any other significant amount or range of amounts.

In some embodiments, the non-immunogenic RNA (especially mRNA) exhibits significantly less innate immunogenicity than standard RNA with the same sequence. In some embodiments, the non-immunogenic RNA (especially mRNA) exhibits an innate immune response that is 2-fold less than its unmodified counterpart. In some embodiments, innate immunogenicity is reduced by a 3-fold factor. In some embodiments, innate immunogenicity is reduced by a 4-fold factor. In some embodiments, innate immunogenicity is reduced by a 5-fold factor. In some embodiments, innate immunogenicity is reduced by a 6-fold factor. In some embodiments, innate immunogenicity is reduced by a 7-fold factor. In some embodiments, innate immunogenicity is reduced by a 8-fold factor. In some embodiments, innate immunogenicity is reduced by a 9-fold factor. In some embodiments, innate immunogenicity is reduced by a 10-fold factor. In some embodiments, innate immunogenicity is reduced by a 15-fold factor. In some embodiments, innate immunogenicity is reduced by a 20-fold factor. In some embodiments, innate immunogenicity is reduced by a 50-fold factor. In some embodiments, innate immunogenicity is reduced by a 100-fold factor. In some embodiments, innate immunogenicity is reduced by a 200-fold factor. In some embodiments, innate immunogenicity is reduced by a 500-fold factor. In some embodiments, innate immunogenicity is reduced by a 1000-fold factor. In some embodiments, innate immunogenicity is reduced by a 2000-fold factor.

The term “exhibits significantly less innate immunogenicity” refers to a detectable decrease in innate immunogenicity. In some embodiments, the term refers to a decrease such that an effective amount of the non-immunogenic RNA (especially mRNA) can be administered without triggering a detectable innate immune response. In some embodiments, the term refers to a decrease such that the non-immunogenic RNA (especially mRNA) can be repeatedly administered without eliciting an innate immune response sufficient to detectably reduce production of the protein encoded by the non-immunogenic RNA. In some embodiments, the decrease is such that the non-immunogenic RNA (especially mRNA) can be repeatedly administered without eliciting an innate immune response sufficient to eliminate detectable production of the protein encoded by the non-immunogenic RNA.

In certain embodiments of the present disclosure, the nucleic acid (e.g., DNA or RNA) in the nucleic acid (e.g., DNA or RNA) compositions (e.g., in the nucleic acid (e.g., DNA or RNA) particles) described herein is at a concentration from about 0.002 mg/mL to about 5 mg/mL, from about 0.002 mg/mL to about 2 mg/mL, from about 0.005 mg/mL to about 2 mg/mL, from about 0.01 mg/mL to about 1 mg/mL, from about 0.05 mg/mL to about 0.5 mg/mL or from about 0.1 mg/mL to about 0.5 mg/mL. In specific embodiments, the nucleic acid (e.g., DNA or RNA) is at a concentration from about 0.005 mg/mL to about 0.1 mg/mL, from about 0.005 mg/mL to about 0.09 mg/mL, from about 0.005 mg/mL to about 0.08 mg/mL, from about 0.005 mg/mL to about 0.07 mg/mL, from about 0.005 mg/mL to about 0.06 mg/mL, or from about 0.005 mg/mL to about 0.05 mg/mL.

### Lipid Particles (Such as Lipid Nanoparticles)

Different types of nucleic acid (e.g., DNA or RNA) containing particles have been described previously to be suitable for delivery of RNA in particulate form (e.g. Kaczmarek, J. C. et al., 2017, Genome Medicine 9, 60). For non-viral nucleic acid (e.g., DNA or RNA) delivery vehicles, nanoparticle encapsulation of nucleic acid (e.g., DNA or RNA) physically protects nucleic acid from degradation and, depending on the specific chemistry, can aid in cellular uptake and endosomal escape.

Electrostatic interactions between positively charged molecules such as polymers and lipids and negatively charged nucleic acid (such as RNA) are involved in particle formation. This results in complexation and spontaneous formation of nucleic acid particles. During the manufacturing process, introduction of an aqueous solution of nucleic acid (e.g., DNA or RNA) to an ethanolic lipid mixture containing a cationically ionizable lipid at pH of, e.g., 5 leads to an electrostatic interaction between the negatively charged nucleic acid (e.g., DNA or RNA) drug substance and the positively charged cationically ionizable lipid. This electrostatic interaction leads to particle formation coincident with efficient encapsulation of nucleic acid (e.g., DNA or RNA) drug substance. After nucleic acid encapsulation, adjustment of the medium surrounding the resulting nucleic acid particles (e.g., RNA-LNPs) to, e.g., pH 7-8 results in neutralization of the surface charge on the particles (e.g., LNPs). When all other variables are held constant, charge-neutral particles display longer in vivo circulation lifetimes and better delivery to hepatocytes compared to charged particles, which are cleared rapidly by the reticuloendothelial system. Upon endosomal uptake, the low pH of the endosome renders the particles (e.g., LNPs) fusogenic and allows for release of the nucleic acid (such as RNA) into the cytosol of the target cell.

In the context of the present disclosure, the term “particle” relates to a structured entity formed by molecules or molecule complexes, in particular particle forming compounds. In some embodiments, the particle contains an envelope (e.g., one or more layers or lamellas) made of one or more types of amphiphilic substances (e.g., amphiphilic lipids, amphiphilic polymers, and/or amphiphilic proteins/polypeptides). In this context, the expression “amphiphilic substance” means that the substance possesses both hydrophilic and lipophilic properties. The envelope may also comprise additional substances (e.g., additional lipids) which do not have to be amphiphilic. Thus, the particle may be a monolamellar or multilamellar structure, wherein the substances constituting the one or more layers or lamellas comprise one or more types of amphiphilic substances (in particular selected from the group consisting of amphiphilic lipids, amphiphilic polymers, and/or amphiphilic proteins/polypeptides) optionally in combination with additional substances (e.g., additional lipids) which do not have to be amphiphilic. In some embodiments, the term “particle” relates to a micro- or nano-sized structure, such as a micro- or nano-sized compact structure. In this respect, the term “micro-sized” means that all three external dimensions of the particle are in the microscale, i.e., between 1 and 5 μm. According to the present disclosure, the term “particle” includes lipoplex particles (LPXs), lipid nanoparticles (LNPs), polyplex particles, lipopolyplex particles, virus-like particles (VLPs), and mixtures thereof (e.g., a mixture of two or more of particle types, such as a mixture of LPXs and VLPs or a mixture of LNPs and VLPs).

A “nucleic acid particle”, such as an “RNA particle”, can be used to deliver nucleic acid (such as RNA, in particular mRNA) to a target site of interest (e.g., cell, tissue, organ, and the like). A nucleic acid particle, such as an RNA particle according to the present disclosure may be formed from at least one cationic or cationically ionizable lipid (such as DODMA), at least one conjugate of a POX and/or POZ polymer and one or more hydrophobic chains (such as a conjugate disclosed herein, in particular a conjugate of formula (II), (II′), (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48) disclosed herein, or a conjugate of formula (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56) disclosed herein), and nucleic acid (in particular RNA, such as mRNA), and optionally one or more additional lipids (such as DOPE or DOPE and cholesterol). Nucleic acid particles include lipid nanoparticle (LNP)-based and lipoplex (LPX)-based formulations.

Without intending to be bound by any theory, it is believed that the cationic or cationically ionizable lipid and the conjugate of a POX and/or POZ polymer and one or more hydrophobic chains and, if present, additional lipids combine together with the nucleic acid (in particular RNA, such as mRNA) to form aggregates, wherein the nucleic acid is bound to the lipid matrix, and this spontaneous aggregation results in colloidally stable particles.

In some embodiments, particles described herein further comprise at least one lipid or lipid-like material other than a cationic or cationically ionizable lipid.

In some embodiments, nucleic acid particles (especially RNA particles such as RNA LNPs (e.g., mRNA particles such as mRNA LNPs)) comprise more than one type of nucleic acid molecules, where the molecular parameters of the nucleic acid molecules may be similar or different from each other, like with respect to molar mass or fundamental structural elements such as molecular architecture, capping, coding regions or other features,

As used in the present disclosure, “nanoparticle” refers to a particle comprising nucleic acid (especially RNA, such as mRNA) as described herein and at least one cationic lipid, wherein all three external dimensions of the particle are in the nanoscale, i.e., at least about 1 nm and below about 1000 nm (preferably, between 10 and 990 nm, such as between 15 and 900 nm, between 20 and 800 nm, between 30 and 700 nm, between 40 and 600 nm, or between 50 and 500 nm). Preferably, the longest and shortest axes do not differ significantly. Preferably, the size of a particle is its diameter. Preferably, the particle has an average diameter suitable for intravenous administration.

Nucleic acid particles described herein (especially RNA LNPs) may exhibit a polydispersity index (PDI) less than about 0.5, less than about 0.4, less than about 0.3, less than about 0.2, less than about 0.1, or less than about 0.05. By way of example, the nucleic acid particles can exhibit a polydispersity index in a range of about 0.01 to about 0.4 or about 0.1 to about 0.3.

In the context of the present disclosure, the term “lipoplex particle” relates to a particle that contains an amphiphilic lipid, in particular cationic amphiphilic lipid, and nucleic acid (especially RNA such as mRNA) as described herein. Electrostatic interactions between positively charged liposomes (made from one or more amphiphilic lipids, in particular cationic amphiphilic lipids) and negatively charged nucleic acid (especially RNA such as mRNA) results in complexation and spontaneous formation of nucleic acid lipoplex particles. Positively charged liposomes may be generally synthesized using a cationic amphiphilic lipid, such as DOTMA, and additional lipids, such as DOPE. In some embodiments, a nucleic acid (especially RNA such as mRNA) lipoplex particle is a nanoparticle.

The term “lipid nanoparticle” relates to a nano-sized lipid containing particle.

In the context of the present disclosure, the term “polyplex particle” relates to a particle that contains an amphiphilic polymer, in particular a cationic amphiphilic polymer, and nucleic acid (especially RNA such as mRNA) as described herein. Electrostatic interactions between positively charged cationic amphiphilic polymers and negatively charged nucleic acid (especially RNA such as mRNA) results in complexation and spontaneous formation of nucleic acid polyplex particles. Positively charged amphiphilic polymers suitable for the preparation of polyplex particle include protamine, polyethyleneimine, poly-L-lysine, poly-L-arginine and histone. In some embodiments, a nucleic acid (especially RNA such as mRNA) polyplex particle is a nanoparticle.

The term “lipopolyplex particle” relates to particle that contains amphiphilic lipid (in particular cationic amphiphilic lipid) as described herein, amphiphilic polymer (in particular cationic amphiphilic polymer) as described herein, and nucleic acid (especially RNA such as mRNA) as described herein. In some embodiments, a nucleic acid (especially RNA such as mRNA) lipopolyplex particle is a nanoparticle.

The term “virus-like particle” (abbreviated herein as VLP) refers to a molecule that closely resembles a virus, but which does not contain any genetic material of said virus and, thus, is non-infectious.

Preferably, VLPs contain nucleic acid (preferably RNA) as described herein, said nucleic acid (preferably RNA) being heterologous to the virus(es) from which the VLPs are derived. VLPs can be synthesized through the individual expression of viral structural proteins, which can then self-assemble into the virus-like structure. In some embodiments, combinations of structural capsid proteins from different viruses can be used to create recombinant VLPs. VLPs can be produced from components of a wide variety of virus families including Hepatitis B virus (HBV) (small HBV derived surface antigen (HBsAg)), Parvoviridae (e.g., adeno-associated virus), Papillomaviridae (e.g., HPV), Retroviridae (e.g., HIV), Flaviviridae (e.g., Hepatitis C virus) and bacteriophages (e.g. Qβ, AP205).

The term “nucleic acid containing particle” relates to a particle as described herein to which nucleic acid (especially RNA such as mRNA) is bound. In this respect, the nucleic acid (especially RNA such as mRNA) may be adhered to the outer surface of the particle (surface nucleic acid (especially surface RNA such as surface mRNA)) and/or may be contained in the particle (encapsulated nucleic acid (especially encapsulated RNA such as encapsulated mRNA)).

In some embodiments, the particles described herein have a size (preferably a diameter, i.e., double the radius such as double the radius of gyration (R_{g}) value or double the hydrodynamic radius) in the range of about 10 to about 2000 nm, such as at least about 15 nm (preferably at least about 20 nm, at least about 25 nm, at least about 30 nm, at least about 35 nm, at least about 40 nm, at least about 45 nm, at least about 50 nm, at least about 55 nm, at least about 60 nm, at least about 65 nm, at least about 70 nm, at least about 75 nm, at least about 80 nm, at least about 85 nm, at least about 90 nm, at least about 95 nm, or at least about 100 nm) and/or at most 1900 nm (preferably at most about 1900 nm, at most about 1800 nm, at most about 1700 nm, at most about 1600 nm, at most about 1500 nm, at most about 1400 nm, at most about 1300 nm, at most about 1200 nm, at most about 1100 nm, at most about 1000 nm, at most about 950 nm, at most about 900 nm, at most about 850 nm, at most about 800 nm, at most about 750 nm, at most about 700 nm, at most about 650 nm, at most about 600 nm, at most about 550 nm, or at most about 500 nm), preferably in the range of about 20 to about 1500 nm, such as about 30 to about 1200 nm, about 40 to about 1100 nm, about 50 to about 1000 nm, about 60 to about 900 nm, about 70 to 800 nm, about 80 to 700 nm, about 90 to 600 nm, or about 50 to 500 nm or about 100 to 500 nm, such as in the range of 10 to 1000 nm, 15 to 500 nm, 20 to 450 nm, 25 to 400 nm, 30 to 350 nm, 40 to 300 nm, 50 to 250 nm, 60 to 200 nm, or 70 to 150 nm.

In some embodiments, the particles (e.g., LNPs and LPXs) described herein have an average diameter that in some embodiments ranges from about 50 nm to about 1000 nm, from about 50 nm to about 800 nm, from about 50 nm to about 700 nm, from about 50 nm to about 600 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 100 nm to about 1000 nm, from about 100 nm to about 800 nm, from about 100 nm to about 700 nm, from about 100 nm to about 600 nm, from about 100 nm to about 500 nm, from about 100 nm to about 450 nm, from about 100 nm to about 400 nm, from about 100 nm to about 350 nm, from about 100 nm to about 300 nm, from about 100 nm to about 250 nm, from about 100 nm to about 200 nm, from about 150 nm to about 1000 nm, from about 150 nm to about 800 nm, from about 150 nm to about 700 nm, from about 150 nm to about 600 nm, from about 150 nm to about 500 nm, from about 150 nm to about 450 nm, from about 150 nm to about 400 nm, from about 150 nm to about 350 nm, from about 150 nm to about 300 nm, from about 150 nm to about 250 nm, from about 150 nm to about 200 nm, from about 200 nm to about 1000 nm, from about 200 nm to about 800 nm, from about 200 nm to about 700 nm, from about 200 nm to about 600 nm, from about 200 nm to about 500 nm, from about 200 nm to about 450 nm, from about 200 nm to about 400 nm, from about 200 nm to about 350 nm, from about 200 nm to about 300 nm, or from about 200 nm to about 250 nm.

With respect to nucleic acid (e.g., DNA or RNA) lipid particles (especially RNA LNPs such as mRNA LNPs), the N/P ratio gives the ratio of the nitrogen groups in the lipid to the number of phosphate groups in the nucleic acid (e.g., DNA or RNA). It is correlated to the charge ratio, as the nitrogen atoms (depending on the pH) are usually positively charged and the phosphate groups are negatively charged. The N/P ratio, where a charge equilibrium exists, depends on the pH. Lipid formulations are frequently formed at N/P ratios larger than four up to twelve, because positively charged nanoparticles are considered favorable for transfection. In that case, nucleic acid (e.g., DNA or RNA) is considered to be completely bound to nanoparticles.

Nucleic acid particles (especially RNA LNPs such as mRNA LNPs) described herein can be prepared using a wide range of methods that may involve obtaining a colloid from at least one cationic or cationically ionizable lipid and mixing the colloid with nucleic acid to obtain nucleic acid particles.

For the preparation of colloids comprising at least one cationic or cationically ionizable lipid methods are applicable herein that are conventionally used for preparing liposomal vesicles and are appropriately adapted. The most commonly used methods for preparing liposomal vesicles share the following fundamental stages: (i) lipids dissolution in organic solvents, (ii) drying of the resultant solution, and (iii) hydration of dried lipid (using various aqueous media).

In the film hydration method, lipids are firstly dissolved in a suitable organic solvent, and dried down to yield a thin film at the bottom of the flask. The obtained lipid film is hydrated using an appropriate aqueous medium to produce a liposomal dispersion. Furthermore, an additional downsizing step may be included.

Reverse phase evaporation is an alternative method to the film hydration for preparing liposomal vesicles that involves formation of a water-in-oil emulsion between an aqueous phase and an organic phase containing lipids. A brief sonication of this mixture is required for system homogenization. The removal of the organic phase under reduced pressure yields a milky gel that turns subsequently into a liposomal suspension.

The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Generally, the nucleic acid (especially RNA such as mRNA) lipoplex particles described herein are obtainable by adding nucleic acid (especially RNA such as mRNA) to a colloidal liposome dispersion. Using the ethanol injection technique, such colloidal liposome dispersion is, in some embodiments, formed as follows: an ethanol solution comprising lipids, such as cationically ionizable lipids and additional lipids, is injected into an aqueous solution under stirring. In some embodiments, the nucleic acid (especially RNA such as mRNA) lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

Other methods having organic solvent free characteristics may also be used according to the present disclosure for preparing a colloid.

Particles (such as LNPs) typically comprise four particle forming components: one or more cationic or cationically ionizable lipids, one or more neutral lipids such as phospholipids, one or more steroids such as cholesterol, and one or more polymer conjugated lipids (in particular, a conjugate of a POX and/or POZ polymer and one or more hydrophobic chains as disclosed herein, especially a conjugate of formula (II), (II′), (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48) disclosed herein, or a conjugate of formula (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56) disclosed herein). Each component is responsible for payload protection, and enables effective intracellular delivery. Particles (such as LNPs) may be prepared by mixing lipids dissolved in ethanol rapidly with nucleic acid (in particular RNA such as mRNA) in an aqueous buffer.

Different types of nucleic acid containing particles have been described previously to be suitable for delivery of nucleic acid in particulate form (cf., e.g., Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

In certain embodiments, the compositions (in particular particles, such as LNPs) comprising nucleic acid (e.g., DNA or RNA), at least one cationic or cationically ionizable lipid described herein, and the conjugate of a POX and/or POZ polymer and one or more hydrophobic chains as disclosed herein (especially a conjugate of formula (II), (II′), (IIa), (IIa), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg*), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48) disclosed herein, or a conjugate of formula (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56) disclosed herein) further comprise one or more additional lipids.

In some embodiments, the compositions (in particular particles, such as LNPs) comprising nucleic acid (e.g., RNA), at least one cationic or cationically ionizable lipid described herein, and the conjugate of a POX and/or POZ polymer and one or more hydrophobic chains as disclosed herein (especially a conjugate of formula (II), (II′), (IIa), (IIa′), (IIb), (IIb), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg*), (IVh), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48) disclosed herein, or a conjugate of formula (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56) disclosed herein) are prepared by (a) preparing a nucleic acid solution (e.g., an RNA solution) containing water and a first buffer system; (b) preparing an ethanolic solution comprising the cationically ionizable lipid, the conjugate of a POX and/or POZ polymer and one or more hydrophobic chains, and, if present, one or more additional lipids; (c) mixing the nucleic acid solution prepared under (a) with the ethanolic solution prepared under (b), thereby preparing a first intermediate formulation comprising the particles (e.g., LNPs) dispersed in a first aqueous phase comprising the first buffer system; and (d) filtrating the first intermediate formulation prepared under (c) using a final aqueous buffer solution comprising the final buffer system, thereby preparing the formulation comprising particles (e.g., LNPs) dispersed in a final aqueous phase comprising the final buffer system. After step (c) one or more steps selected from diluting and filtrating, such as tangential flow filtrating or diafiltrating, can follow.

In an alternative embodiment, the compositions (in particular particles, such as LNPs) comprising nucleic acid (e.g., RNA), at least one cationic or cationically ionizable lipid described herein, and the conjugate of a POX and/or POZ polymer and one or more hydrophobic chains as disclosed herein (especially a conjugate of formula (II), (II), (IIa), (IIa), (IIb), (IIb), (IIc), (IId), (IIe), (IIf), (IIg), (IIh), (IIi′), (IIj′), (III), (III), (IIIa), (IIIa′), (IIIb), (IIIb), (IV), (IV), (IVa), (IVb), (IVc), (IVd′), (IVc), (IVf), (IVg), (IVh), (V), (V′), (VI), (VI), (VIa), (VIa), (VII), (VII), (VIIa), (VIIa), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48) disclosed herein, or a conjugate of formula (IIc), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56) disclosed herein) are prepared by (a) preparing liposomes or a colloidal preparation of the cationically ionizable lipid and, if present, one or more additional lipids in an aqueous phase: (b′) preparing a nucleic acid solution (e.g., an RNA solution) containing water and a buffering system; and (c′) mixing the liposomes or colloidal preparation prepared under (a) with the nucleic acid solution prepared under (b). After step (c′) one or more steps selected from diluting and filtrating, such as tangential flow filtrating, can follow.

The present disclosure describes compositions which comprise particles comprising nucleic acid (e.g., DNA or RNA, especially LNPs comprising RNA), at least one cationic or cationically ionizable lipid, and the conjugate of a POX and/or POZ polymer and one or more hydrophobic chains as disclosed herein (especially a conjugate of formula (II), (II′), (IIa), (IIa), (IIb), (IIb), (IIc), (IId), (IIe), (IIf), (IIg), (IIh), (IIi′), (IIj′), (III), (III), (IIIa), (IIIa), (IIIb), (IIIb), (IV), (IV), (IVa), (IVb), (IVc), (IVd′), (IVe), (IVf), (IVg′), (IVh), (V), (V′), (VI), (VI′), (VIa), (VIa), (VII), (VII′), (VIIa), (VIIa), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48) disclosed herein, or a conjugate of formula (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56) disclosed herein) which associate with the nucleic acid (e.g., DNA or RNA) to form nucleic acid particles. The nucleic acid (e.g., DNA or RNA) particles may comprise nucleic acid which is complexed in different forms by non-covalent interactions to the particle. The particles described herein are not viral particles, in particular infectious viral particles, i.e., they are not able to virally infect cells.

Suitable cationically ionizable lipids are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

In particulate formulation, it is possible that each nucleic acid (e.g., DNA or RNA) species is separately formulated as an individual particulate formulation. In that case, each individual particulate formulation will comprise one nucleic acid (e.g., DNA or RNA) species. The individual particulate formulations may be present as separate entities, e.g. in separate containers. Such formulations are obtainable by providing each nucleic acid (e.g., DNA or RNA) species separately (typically each in the form of an nucleic acid-containing solution) together with a particle-forming agent, thereby allowing the formation of particles. Respective particles will contain exclusively the specific nucleic acid (e.g., DNA or RNA) species that is being provided when the particles are formed (individual particulate formulations). In some embodiments, a composition such as a pharmaceutical composition comprises more than one individual particle formulation. Respective pharmaceutical compositions are referred to as mixed particulate formulations. Mixed particulate formulations according to the present disclosure are obtainable by forming, separately, individual particulate formulations, followed by a step of mixing of the individual particulate formulations. By the step of mixing, a formulation comprising a mixed population of nucleic acid (e.g., DNA or RNA)-containing particles is obtainable. Individual particulate populations may be together in one container, comprising a mixed population of individual particulate formulations. Alternatively, it is possible that all nucleic acid (e.g., DNA or RNA) species of the pharmaceutical composition are formulated together as a combined particulate formulation. Such formulations are obtainable by providing a combined formulation (typically combined solution) of all nucleic acid (e.g., DNA or RNA) species together with a particle-forming agent, thereby allowing the formation of particles. As opposed to a mixed particulate formulation, a combined particulate formulation will typically comprise particles which comprise more than one nucleic acid (e.g., DNA or RNA) species. In a combined particulate composition different nucleic acid (e.g., DNA or RNA) species are typically present together in a single particle.

### Lipids

The terms “lipid” and “lipid-like material” are broadly defined herein as molecules which comprise one or more hydrophobic moieties or groups and optionally also one or more hydrophilic moieties or groups. Molecules comprising hydrophobic moieties and hydrophilic moieties are also frequently denoted as amphiphiles. Lipids are usually insoluble or poorly soluble in water, but soluble in many organic solvents. In an aqueous environment, the amphiphilic nature allows the molecules to self-assemble into organized structures and different phases. One of those phases consists of lipid bilayers, as they are present in vesicles, multilamellar/unilamellar liposomes, or membranes in an aqueous environment. Hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). The hydrophilic groups may comprise polar and/or charged groups and include carbohydrates, phosphate, carboxylic, sulfate, amino, sulfhydryl, nitro, hydroxyl, and other like groups.

Generally, lipids may be divided into eight categories: fatty acids, glycerolipids, glycerophospholipids, sphingolipids, saccharolipids, polyketides (derived from condensation of ketoacyl subunits), sterol lipids and prenol lipids (derived from condensation of isoprene subunits). Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as steroids, i.e., sterol-containing metabolites such as cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Fatty acids, or fatty acid residues are a diverse group of molecules made of a hydrocarbon chain that terminates with a carboxylic acid group; this arrangement confers the molecule with a polar, hydrophilic end, and a nonpolar, hydrophobic end that is insoluble in water. The carbon chain, typically between four and 24 carbons long, may be saturated or unsaturated, and may be attached to functional groups containing oxygen, halogens, nitrogen, and sulfur. If a fatty acid contains a double bond, there is the possibility of either a cis or trans geometric isomerism, which significantly affects the molecule's configuration. Cis-double bonds cause the fatty acid chain to bend, an effect that is compounded with more cis double bonds in the chain. Other major lipid classes in the fatty acid category are the fatty esters and fatty amides.

Glycerolipids are composed of mono-, di-, and tri-substituted glycerols, the best-known being the fatty acid triesters of glycerol, called triglycerides. The word “triacylglycerol” is sometimes used synonymously with “triglyceride”. In these compounds, the three hydroxyl groups of glycerol are each esterified, typically by different fatty acids. Additional subclasses of glycerolipids are represented by glycosylglycerols, which are characterized by the presence of one or more sugar residues attached to glycerol via a glycosidic linkage.

The glycerophospholipids are amphipathic molecules (containing both hydrophobic and hydrophilic regions) that contain a glycerol core linked to two fatty acid-derived “tails” by ester linkages and to one “head” group by a phosphate ester linkage. Examples of glycerophospholipids, usually referred to as phospholipids (though sphingomyelins are also classified as phospholipids) are phosphatidylcholine (also known as PC, GPCho or lecithin), phosphatidylethanolamine (PE or GPEtn) and phosphatidylserine (PS or GPSer).

Sphingolipids are a complex family of compounds that share a common structural feature, a sphingoid base backbone. The major sphingoid base in mammals is commonly referred to as sphingosine. Ceramides (N-acyl-sphingoid bases) are a major subclass of sphingoid base derivatives with an amide-linked fatty acid. The fatty acids are typically saturated or mono-unsaturated with chain lengths from 16 to 26 carbon atoms. The major phosphosphingolipids of mammals are sphingomyelins (ceramide phosphocholines), whereas insects contain mainly ceramide phosphoethanolamines and fungi have phytoceramide phosphoinositols and mannose-containing headgroups. The glycosphingolipids are a diverse family of molecules composed of one or more sugar residues linked via a glycosidic bond to the sphingoid base. Examples of these are the simple and complex glycosphingolipids such as cerebrosides and gangliosides.

Sterol lipids, such as cholesterol and its derivatives, or tocopherol and its derivatives, are an important component of membrane lipids, along with the glycerophospholipids and sphingomyelins.

Saccharolipids describe compounds in which fatty acids are linked directly to a sugar backbone, forming structures that are compatible with membrane bilayers. In the saccharolipids, a monosaccharide substitutes for the glycerol backbone present in glycerolipids and glycerophospholipids. The most familiar saccharolipids are the acylated glucosamine precursors of the Lipid A component of the lipopolysaccharides in Gram-negative bacteria. Typical lipid A molecules are disaccharides of glucosamine, which are derivatized with as many as seven fatty-acyl chains. The minimal lipopolysaccharide required for growth in E. coli is Kdo2-Lipid A, a hexa-acylated disaccharide of glucosamine that is glycosylated with two 3-deoxy-D-manno-octulosonic acid (Kdo) residues.

Polyketides are synthesized by polymerization of acetyl and propionyl subunits by classic enzymes as well as iterative and multimodular enzymes that share mechanistic features with the fatty acid synthases. They comprise a large number of secondary metabolites and natural products from animal, plant, bacterial, fungal and marine sources, and have great structural diversity. Many polyketides are cyclic molecules whose backbones are often further modified by glycosylation, methylation, hydroxylation, oxidation, or other processes.

According to the disclosure, lipids and lipid-like materials may be cationic, anionic or neutral. Neutral lipids or lipid-like materials exist in an uncharged or neutral zwitterionic form at a selected pH.

### Cationic or Cationically Ionizable Lipids

The nucleic acid compositions (especially RNA LNPs) described herein comprise a cationic or cationically ionizable lipid as particle forming agent. Cationic or cationically ionizable lipids contemplated for use herein include any cationic and cationically ionizable lipids or lipid-like materials which are able to electrostatically bind nucleic acid. In some embodiments, cationic and cationically ionizable lipids contemplated for use herein can be associated with nucleic acid, e.g. by forming complexes with the nucleic acid or forming vesicles in which the nucleic acid is enclosed or encapsulated.

As used herein, a “cationic lipid” or “cationic lipid-like material” refers to a lipid or lipid-like material having a net positive charge. Cationic lipids or lipid-like materials bind negatively charged nucleic acid by electrostatic interaction. Generally, cationic lipids possess a hydrophobic (in some embodiments lipophilic) moiety, such as a sterol, an acyl chain, a diacyl or more acyl chains, and the head group of the lipid typically carries the positive charge.

In certain embodiments, a cationic lipid or lipid-like material has a net positive charge only at certain pH, in particular acidic pH, while it has preferably no net positive charge, preferably has no charge, i.e., it is neutral, at a different, preferably higher pH such as physiological pH. This ionizable behavior is thought to enhance efficacy through helping with endosomal escape and reducing toxicity as compared with particles that remain cationic at physiological pH.

As used herein, a “cationically ionizable lipid” refers to a lipid or lipid-like material which has a net positive charge or is neutral, i.e., a lipid which is not permanently cationic. Thus, depending on the pH of the composition in which the cationically ionizable lipid is solved, the cationically ionizable lipid is either positively charged or neutral. For purposes of the present disclosure, such “cationically ionizable” lipids are comprised by the term “cationic lipid” unless contradicted by the circumstances.

Examples of cationic lipids include, but are not limited to N,N-dimethyl-2,3-diolcyloxypropylamine (DODMA), 1,2-di-O-octadecenyl-3-trimethylammonium propane (DOTMA), 3-(N—(N′,N′-dimethylaminocthane)-carbamoyl) cholesterol (DC-Chol), dimethyldioctadecylammonium (DDAB); 1,2-diolcoyl-3-trimethylammonium propane (DOTAP); 1,2-diolcoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-distearyloxy-N,N-dimethyl-3-aminopropane (DSDMA), 2,3-di(tetradecoxy) propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-diolcyloxypropyl-3-dimethyl-hydroxyethyl ammonium bromide (DORIE), and 2,3-diolcoyloxy-N-[2 (spermine carboxamide)ethyl]-N,N-dimethyl-I-propanamium trifluoroacetate (DOSPA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′. 12′-octadecadienoxy) propane (CpLinDMA), N,N-dimethyl-3,4-dioleyloxybenzylamine (DMOBA), 1,2-N,N′-diolcylcarbamyl-3-dimethylaminopropane (DOcarbDAP), 2,3-Dilinolcoyloxy-N,N-dimethylpropylamine (DLinDAP), 1,2-N,N′-Dilinoleylcarbamyl-3-dimethylaminopropane (DLincarbDAP), 1,2-Dilinolcoylcarbamyl-3-dimethylaminopropane (DLinCDAP), 2,2-dilinoleyl-4-dimethylaminomethyl-[1,3]-dioxolane (DLin-K-DMA), 2,2-dilinolcyl-4-dimethylaminoethyl-[1,3]-dioxolane (DLin-K-XTC2-DMA), 2,2-dilinolcyl-4-(2-dimethylaminoethyl)-[1.3]-dioxolane (DLin-KC2-DMA), heptatriaconta-6,9,28,31-tetraen-19-yl-4-(dimethylamino) butanoate (DLin-MC3-DMA), N-(2-Hydroxyethyl)-N,N-dimethyl-2,3-bis(tetradecyloxy)-1-propanaminium bromide (DMRIE), (+)—N-(3-aminopropyl)-N,N-dimethyl-2,3-bis(cis-9-tetradecenyloxy)-1-propanaminium bromide (GAP-DMORIE), (+)—N-(3-aminopropyl)-N,N-dimethyl-2,3-bis(dodecyloxy)-1-propanaminium bromide (GAP-DLRIE), (+)—N-(3-aminopropyl)-N,N-dimethyl-2,3-bis(tetradecyloxy)-1-propanaminium bromide (GAP-DMRIE), N-(2-Aminoethyl)-N,N-dimethyl-2,3-bis(tetradecyloxy)-1-propanaminium bromide (BAE-DMRIE), N-(4-carboxybenzyl)-N,N-dimethyl-2,3-bis(olcoyloxy) propan-1-aminium (DOBAQ), 2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-aminopropyl)amino]butylcarboxamido)ethyl]-3,4-di[oleyloxy]-benzamide (MVL5), 1,2-diolcoyl-sn-glycero-3-ethylphosphocholine (DOEPC), 2,3-bis(dodecyloxy)-N-(2-hydroxyethyl)-N,N-dimethylpropan-1-amonium bromide (DLRIE), N-(2-aminoethyl)-N,N-dimethyl-2,3-bis(tetradecyloxy) propan-1-aminium bromide (DMORIE), di((Z)-non-2-en-1-yl) 8,8′-((((2 (dimethylamino)ethyl)thio) carbonyl)azanediyl)-dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), Di((Z)-non-2-en-1-yl)-9-((4-(dimethylaminobutanoyl)-oxy) heptadecanedioate (L₃₁₉), N-Dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200). Preferred are DODMA, DOTMA, DOTAP, DODAC, and DOSPA. In specific embodiments, the cationic or cationically ionizable lipid is DODMA.

DOTMA is a cationic lipid with a quaternary amine headgroup. The structure of DOTMA may be represented as follows:

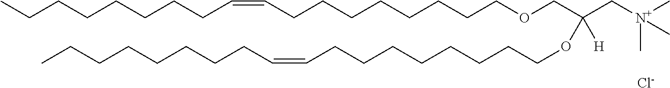 [SMILES: CCCCCCCC/C=C\CCCCCCCCOCC(C[N+](C)(C)C)OCCCCCCCC/C=C\CCCCCCCC ; [Cl-]]

DODMA is an ionizable cationic lipid with a tertiary amine headgroup. The structure of DODMA may be represented as follows:

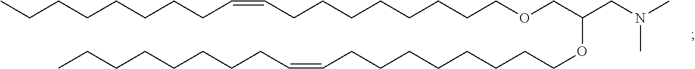 [SMILES: CCCCCCCC/C=C\CCCCCCCCOCC(CN(C)C)OCCCCCCCC/C=C\CCCCCCCC]

In certain embodiments, the composition comprises a cationically ionizable lipid.

In some embodiments, the cationically ionizable lipid comprises a head group which includes at least one nitrogen atom (N) which is positive charged or capable of being protonated, preferably under physiological conditions.

Examples of cationically ionizable lipids are disclosed, for example, in WO 2016/176330 and WO 2018/078053. In some embodiments, the cationically ionizable lipid has the structure of Formula (X):

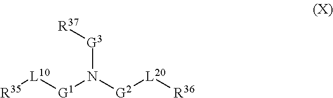

or a pharmaceutically acceptable salt, tautomer, prodrug or stereoisomer thereof, wherein: one of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is —O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₂-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆-24 alkyl or C₆-24 alkenyl;
R³⁷ is H, OR⁵⁰, CN, —C(═O)OR⁴⁰, —OC(═O) R⁴⁰ or —NR⁵⁰C(═O) R⁴⁰;
R⁴⁰ is C₁₋₁₂ alkyl;
R⁵⁰ is H or C₁₋₆ alkyl; and
x is 0, 1 or 2.

In some of the foregoing embodiments of Formula (X), the lipid has one of the following structures (XA) or (XB):

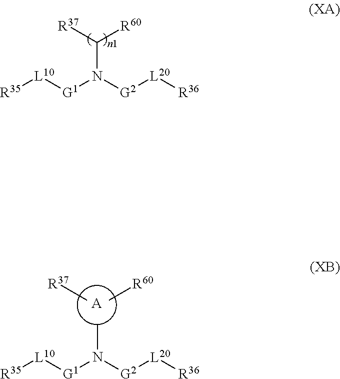

wherein:
A is a 3 to 8-membered cycloalkyl or cycloalkylene group;
R⁶⁰ is, at each occurrence, independently H, OH or C₁-C₂₄ alkyl;
n1 is an integer ranging from 1 to 15.

In some of the foregoing embodiments of Formula (X), the lipid has structure (XA), and in other embodiments, the lipid has structure (XB).

In other embodiments of Formula (X), the lipid has one of the following structures (XC) or (XD):

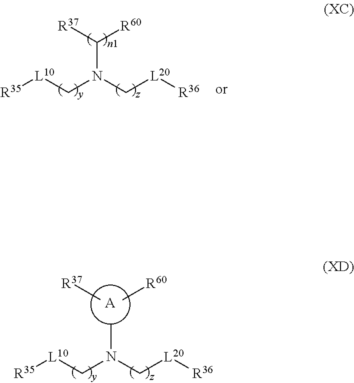

wherein y and z are each independently integers ranging from 1 to 12.

In any of the foregoing embodiments of Formula (X), one of L¹⁰ and L²⁰ is —O(C═O)—. For example, in some embodiments each of L¹⁰ and L²⁰ are —O(C═O)—. In some different embodiments of any of the foregoing, L¹⁰ and L²⁰ are each independently —(C═O)O— or —O(C═O)—. For example, in some embodiments each of L¹⁰ and L²⁰ is —(C═O)O—.

In some different embodiments of Formula (X), the lipid has one of the following structures (XE) or (XF):

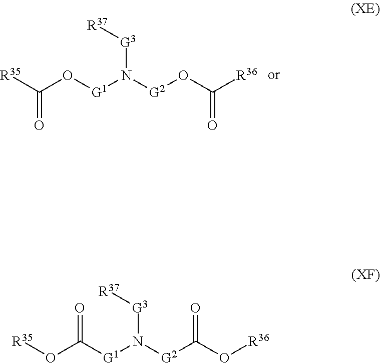

In some of the foregoing embodiments of Formula (X), the lipid has one of the following structures (XG), (XH), (XJ), or (XK):

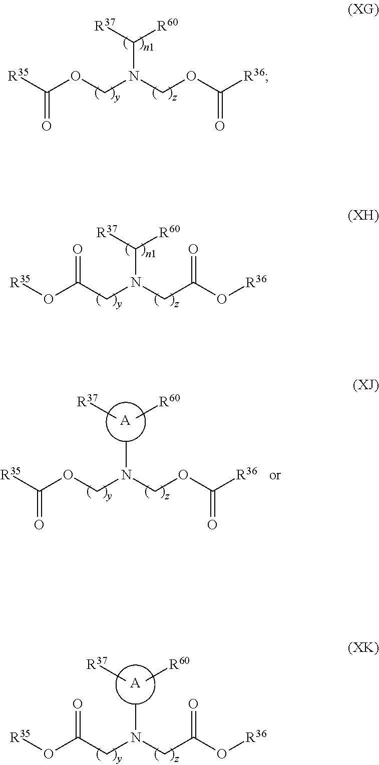

In some of the foregoing embodiments of Formula (X), n1 is an integer ranging from 2 to 12, for example from 2 to 8 or from 2 to 4. For example, in some embodiments, n1 is 3, 4, 5 or 6. In some embodiments, n1 is 3. In some embodiments, n1 is 4. In some embodiments, n1 is 5. In some embodiments, n1 is 6.

In some other of the foregoing embodiments of Formula (X), y and z are each independently an integer ranging from 2 to 10. For example, in some embodiments, y and z are each independently an integer ranging from 4 to 9 or from 4 to 6.

In some of the foregoing embodiments of Formula (X), R⁶⁰ is H. In other of the foregoing embodiments, R⁶⁰ is C₁-C₂₄ alkyl. In other embodiments, R⁶⁰ is OH.

In some embodiments of Formula (X), G³ is unsubstituted. In other embodiments, G³ is substituted. In various different embodiments, G³ is linear C₁-C₂₄ alkylene or linear C₂-C₂₄ alkenylene.

In some other foregoing embodiments of Formula (X), R³⁵ or R³⁶, or both, is C₆-C₂₄ alkenyl. For example, in some embodiments, R³⁵ and R³⁶ each, independently have the following structure:

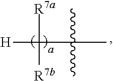

wherein:
R^{7a} and R^{7b} are, at each occurrence, independently H or C₁-C₁₂ alkyl; and
a is an integer from 2 to 12,
wherein R^{7a}, R^{7b} and a are each selected such that R³⁵ and R³⁶ each independently comprise from 6 to 20 carbon atoms. For example, in some embodiments a is an integer ranging from 5 to 9 or from 8 to 12.

In some of the foregoing embodiments of Formula (X), at least one occurrence of R^{7a} is H. For example, in some embodiments, R^{7a} is H at each occurrence. In other different embodiments of the foregoing, at least one occurrence of R^{7b} is C₁-C₈ alkyl. For example, in some embodiments, C₁-C₈ alkyl is methyl, ethyl, n-propyl, iso-propyl, n-butyl, iso-butyl, tert-butyl, n-hexyl or n-octyl.

In different embodiments of Formula (X), R³⁵ or R³⁶, or both, has one of the following structures:

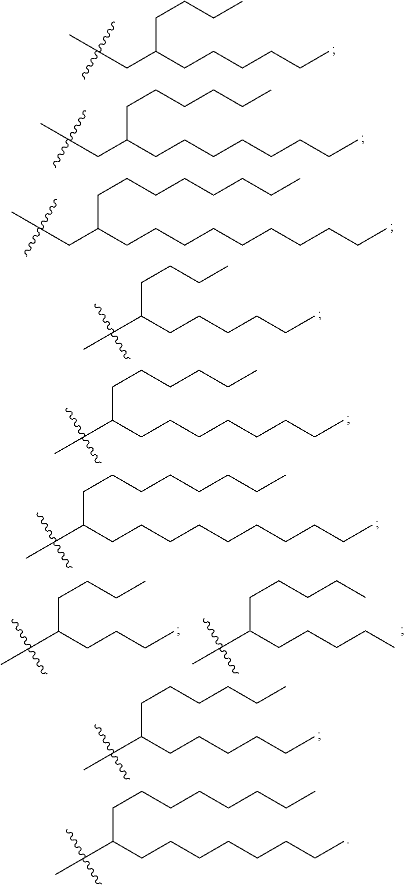

In some of the foregoing embodiments of Formula (X), R³⁷ is OH, CN, —C(═O)OR⁴⁰, —OC(═O) R⁴⁰ or —NHC(═O) R⁴⁰. In some embodiments, R⁴⁰ is methyl or ethyl.

In various different embodiments, the cationic lipid of Formula (X) has one of the structures set forth below.

### Representative Compounds of Formula (X).

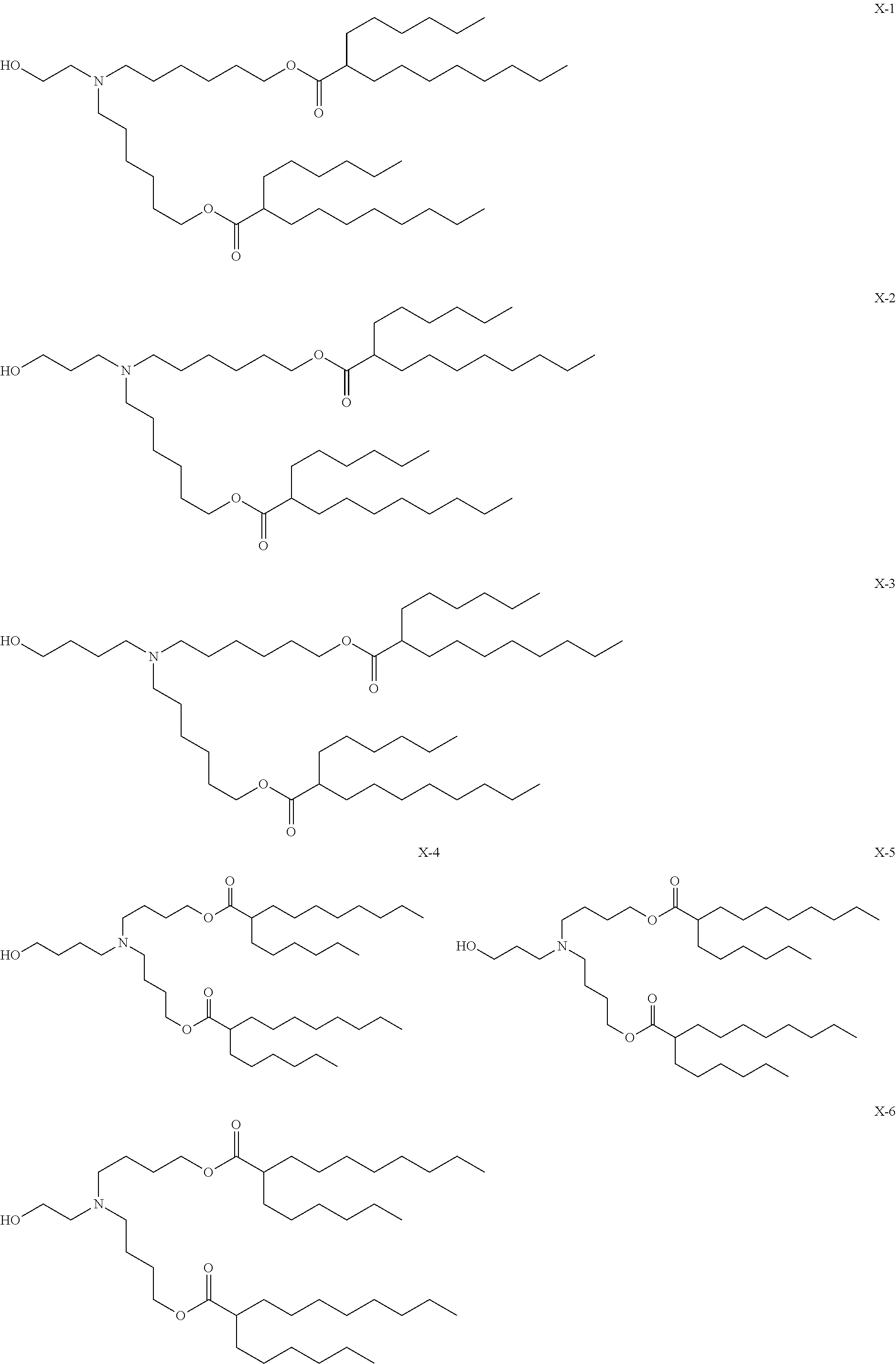 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCN(CCCCO)CCCCOC(=O)C(CCCCCC)CCCCCCCC ; CCCCCCCCC(CCCCCC)C(=O)OCCCCN(CCCO)CCCCOC(=O)C(CCCCCC)CCCCCCCC ; CCCCCCCCC(CCCCCC)C(=O)OCCCCN(CCO)CCCCOC(=O)C(CCCCCC)CCCCCCCC]

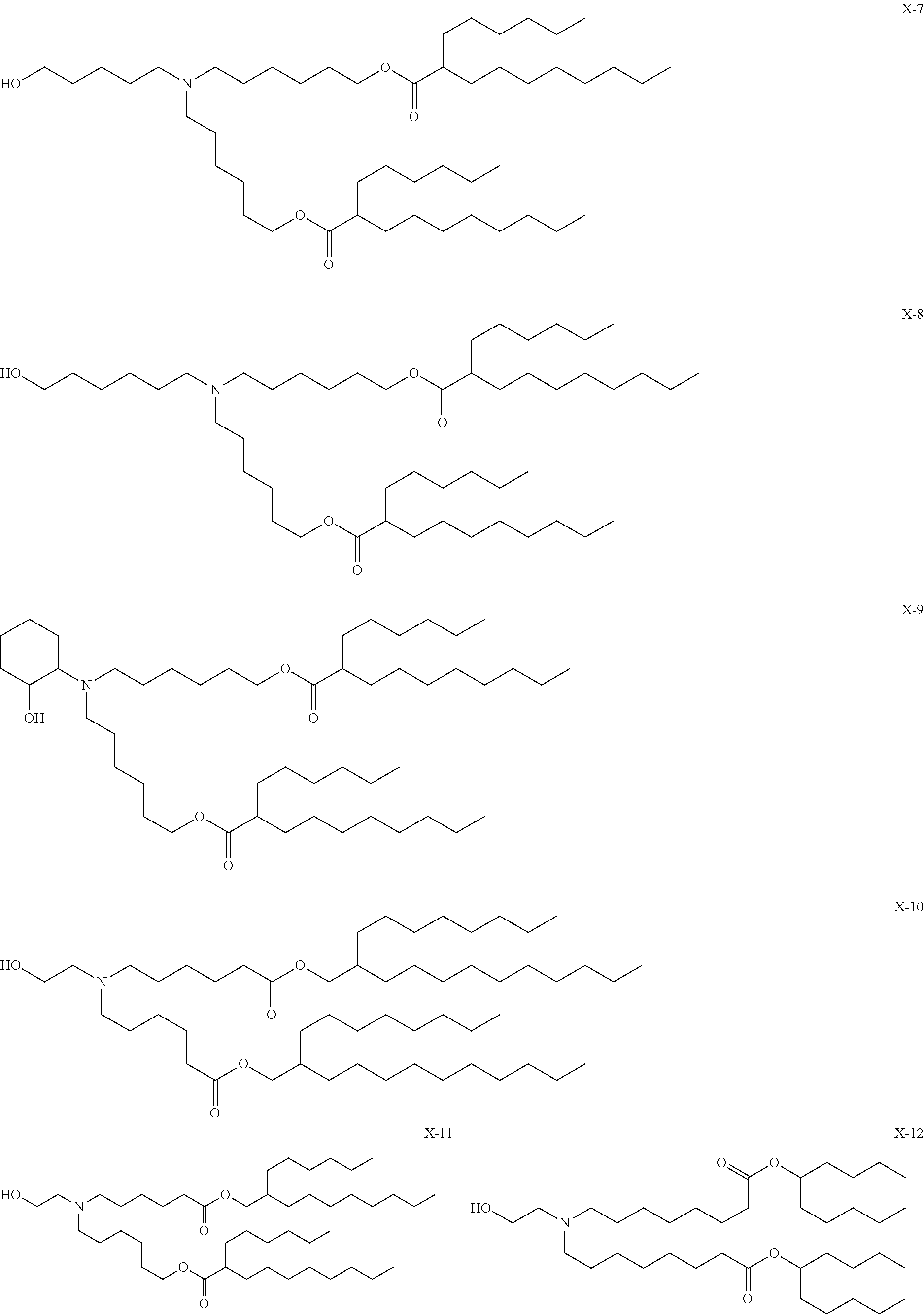 [SMILES: CCCCCCCCC(CCCCCC)C(=O)OCCCCCCN(CCCCCO)CCCCCCOC(=O)C(CCCCCC)CCCCCCCC ; CCCCCCCCC(CCCCCC)C(=O)OCCCCCCN(CCCCCCO)CCCCCCOC(=O)C(CCCCCC)CCCCCCCC ; CCCCCCCCC(CCCCCC)C(=O)OCCCCCCN(CCCCCCOC(=O)C(CCCCCC)CCCCCCCC)C1CCCCC1O ; CCCCCCCCCCC(CCCCCCCC)COC(=O)CCCCCN(CCO)CCCCCC(=O)OCC(CCCCCCCC)CCCCCCCCCC ; CCCCCCCCC(CCCCCC)COC(=O)CCCCCN(CCO)CCCCCCOC(=O)C(CCCCCC)CCCCCCCC ; CCCCCC(CCCC)OC(=O)CCCCCCCN(CCO)CCCCCCCC(=O)OC(CCCC)CCCCC]

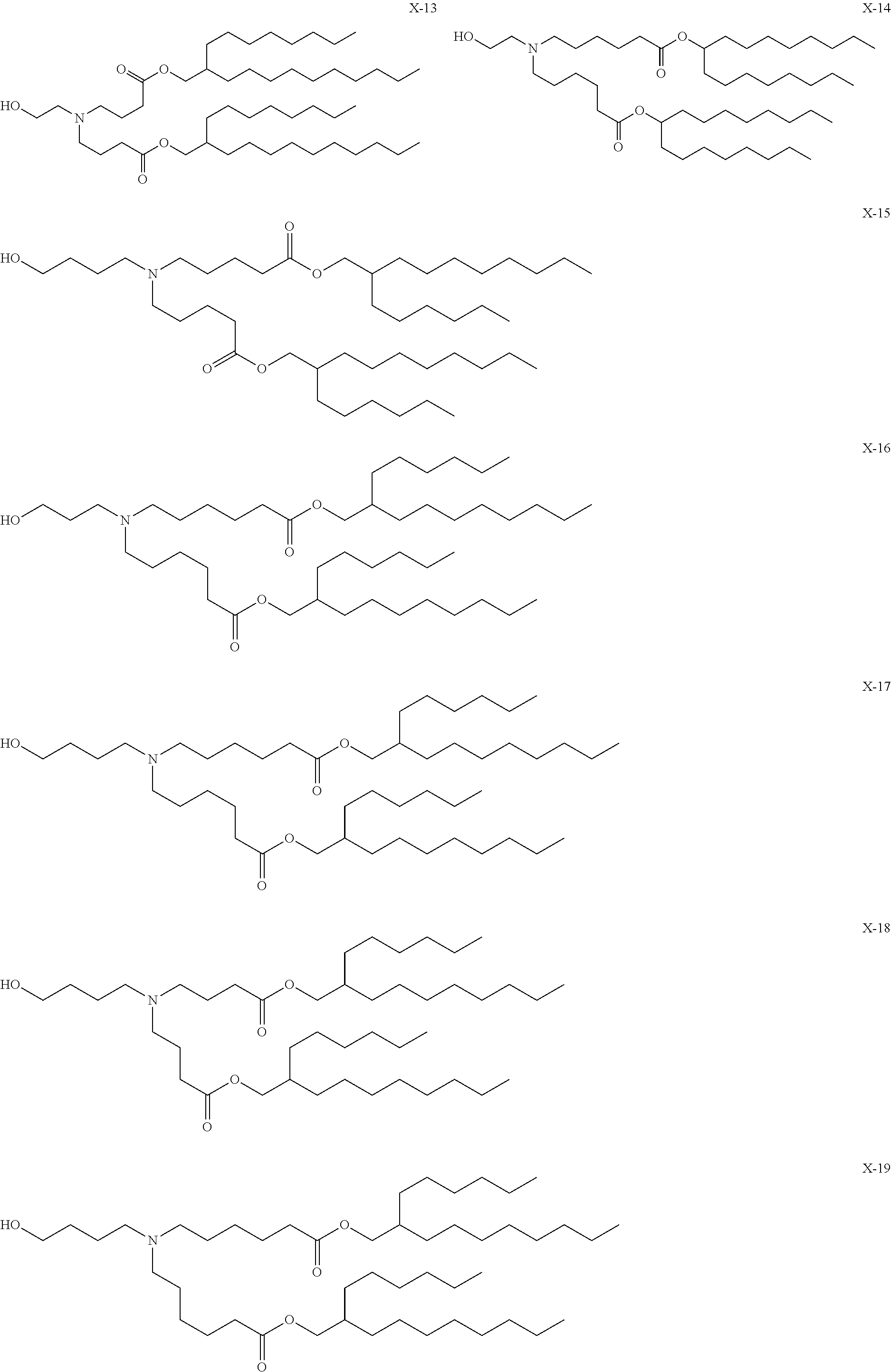

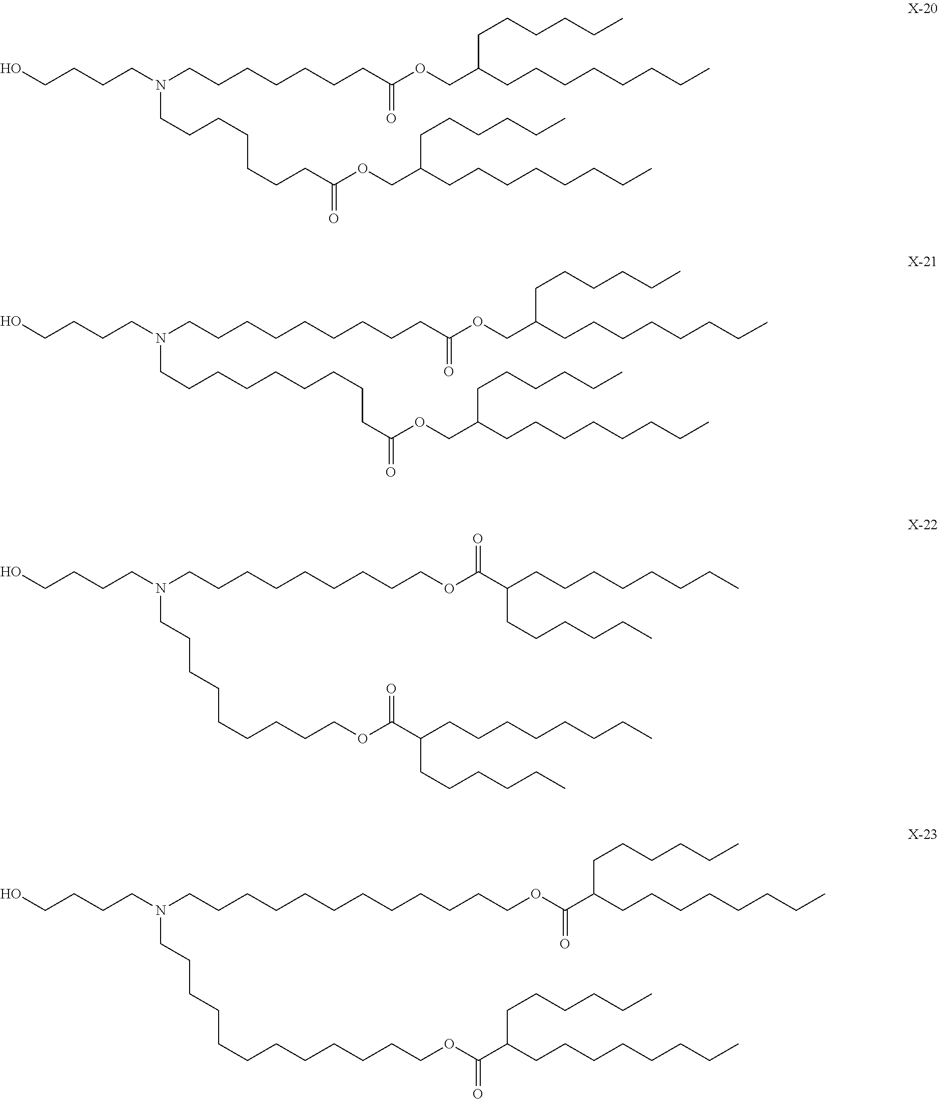 [SMILES: CCCCCCCCC(CCCCCC)COC(=O)CCCCCCCN(CCCCO)CCCCCCCC(=O)OCC(CCCCCC)CCCCCCCC ; CCCCCCCCC(CCCCCC)COC(=O)CCCCCCCCCN(CCCCO)CCCCCCCCCC(=O)OCC(CCCCCC)CCCCCCCC ; CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCCCCCCCN(CCCCO)CCCCCCCCCCCCOC(=O)C(CCCCCC)CCCCCCCC]

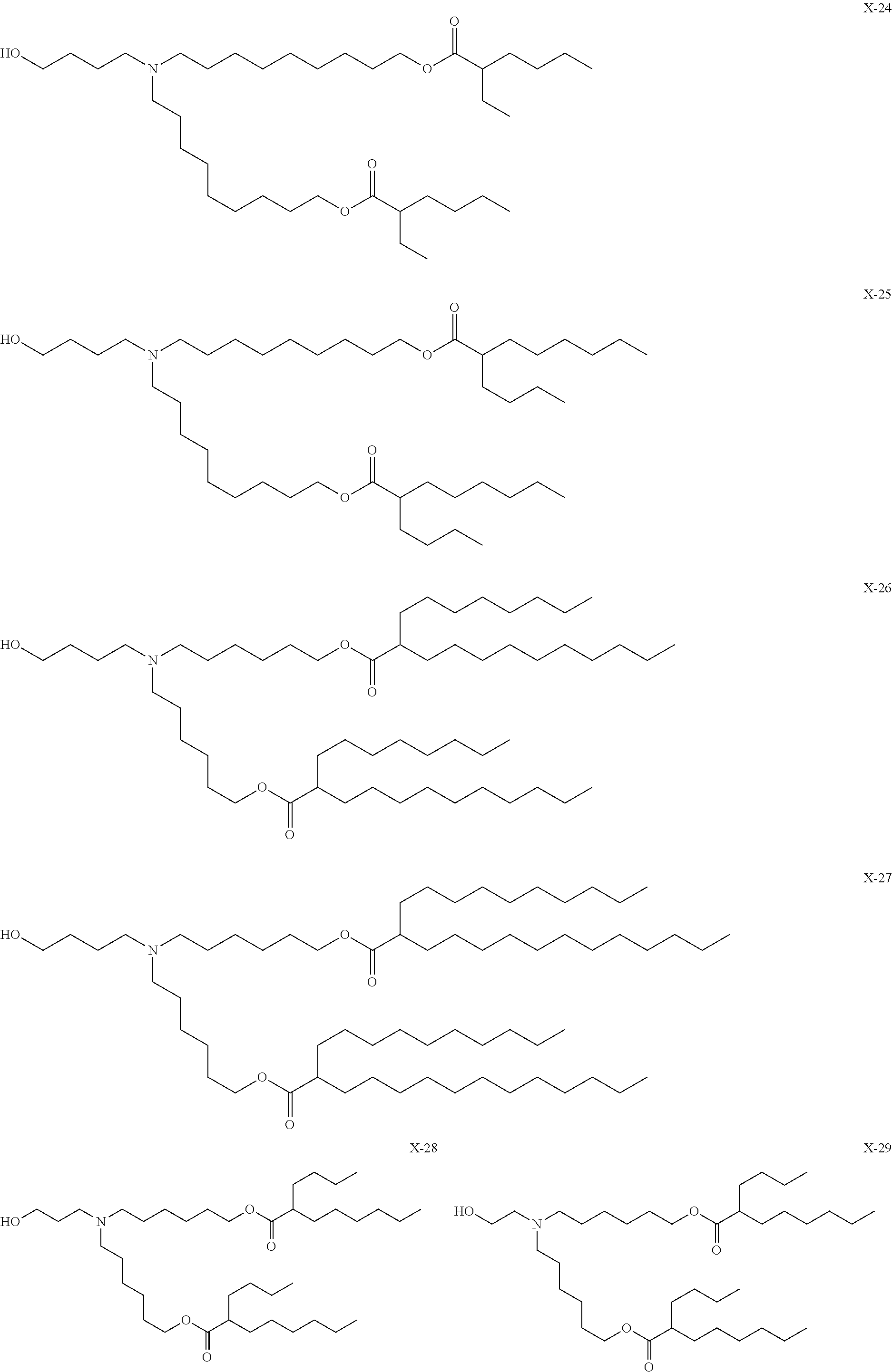 [SMILES: CCCCC(CC)C(=O)OCCCCCCCCCN(CCCCO)CCCCCCCCCOC(=O)C(CC)CCCC ; CCCCCCC(CCCC)C(=O)OCCCCCCCCCN(CCCCO)CCCCCCCCCOC(=O)C(CCCC)CCCCCC ; CCCCCCCCCCC(CCCCCCCC)C(=O)OCCCCCCN(CCCCO)CCCCCCOC(=O)C(CCCCCCCC)CCCCCCCCCC ; CCCCCCCCCCCCC(CCCCCCCCCC)C(=O)OCCCCCCN(CCCCO)CCCCCCOC(=O)C(CCCCCCCCCC)CCCCCCCCCCCC ; CCCCCCC(CCCC)C(=O)OCCCCCCN(CCCO)CCCCCCOC(=O)C(CCCC)CCCCCC ; CCCCCCC(CCCC)C(=O)OCCCCCCN(CCO)CCCCCCOC(=O)C(CCCC)CCCCCC]

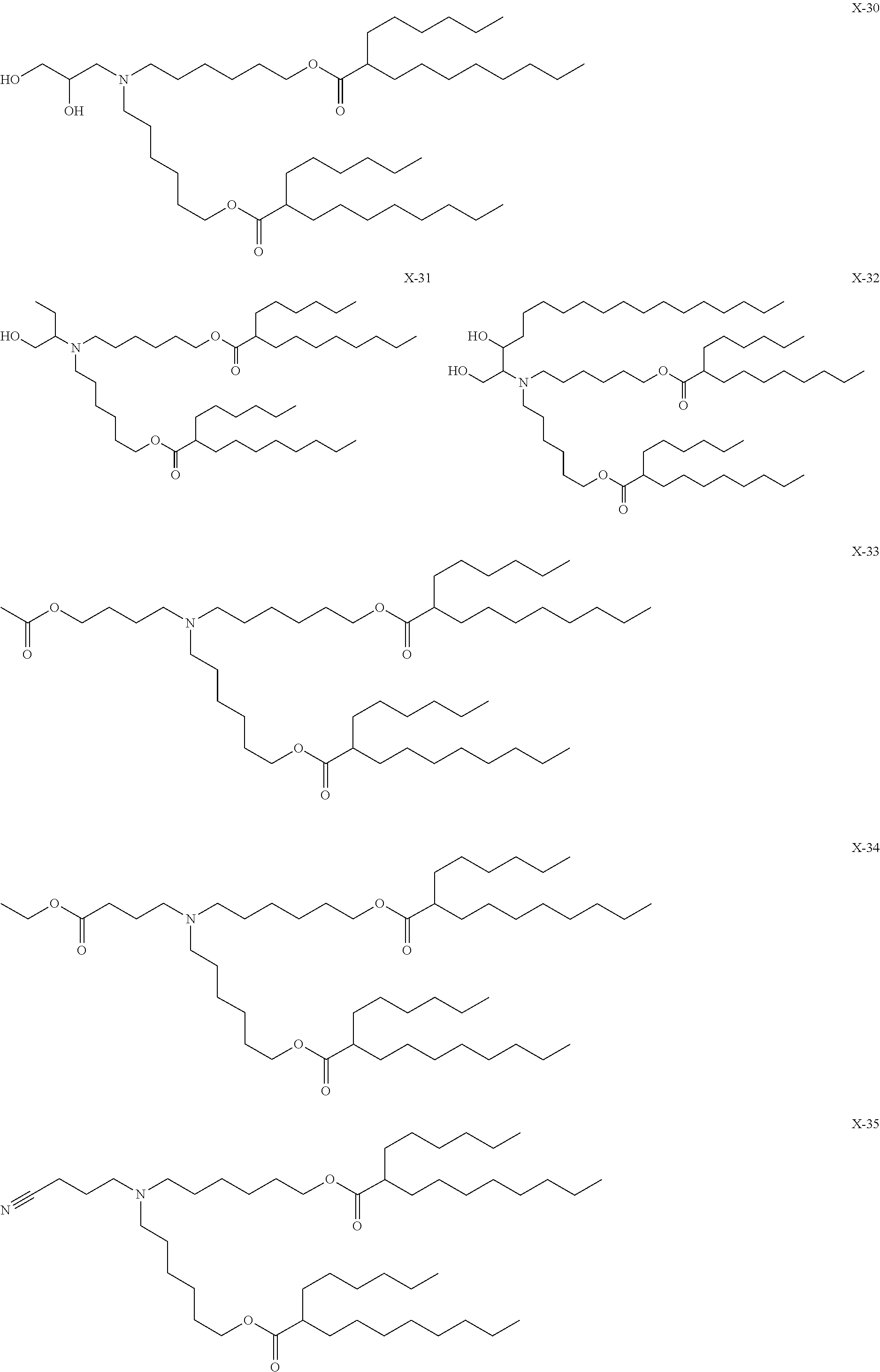

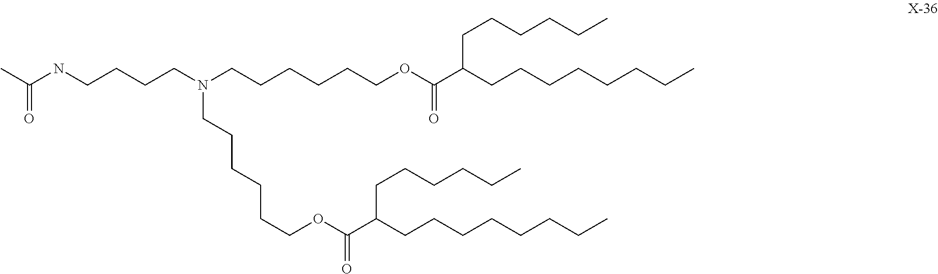

In various different embodiments, the cationically ionizable lipid has one of the structures set forth in the table below.

| No. | Structure |
|---|---|
| A | 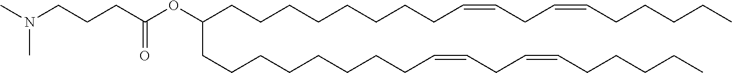 [SMILES: CCCCC/C=C\C/C=C\CCCCCCCCC(CCCCCCCC/C=C\C/C=C\CCCCC)OC(=O)CCCN(C)C] |
| B | 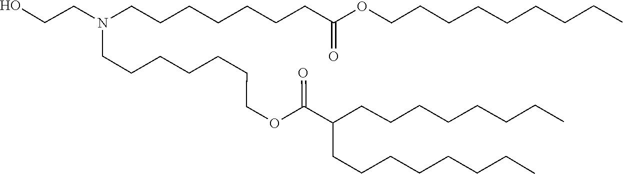 [SMILES: CCCCCCCCCOC(=O)CCCCCCCN(CCO)CCCCCCCOC(=O)C(CCCCCCCC)CCCCCCCC] |
| C | 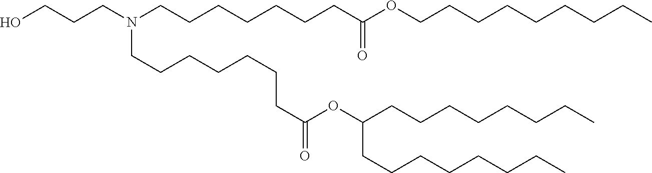 [SMILES: CCCCCCCCCOC(=O)CCCCCCCN(CCCO)CCCCCCCC(=O)OC(CCCCCCCC)CCCCCCCC] |
| D | 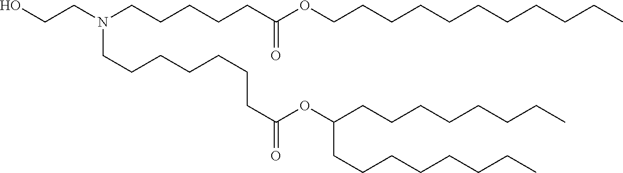 [SMILES: CCCCCCCCCCCOC(=O)CCCCCN(CCO)CCCCCCCC(=O)OC(CCCCCCCC)CCCCCCCC] |
| E | 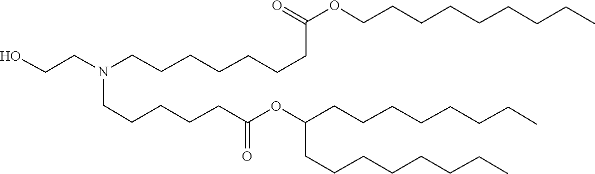 [SMILES: CCCCCCCCCOC(=O)CCCCCCCN(CCO)CCCCCC(=O)OC(CCCCCCCC)CCCCCCCC] |
| F | 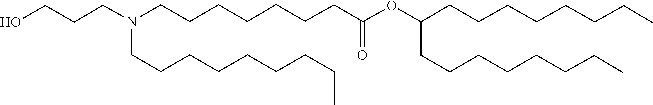 [SMILES: CCCCCCCCCN(CCCO)CCCCCCCC(=O)OC(CCCCCCCC)CCCCCCCC] |
| G | 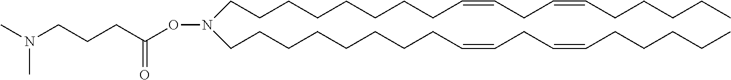 [SMILES: CCCCC/C=C\C/C=C\CCCCCCCCN(CCCCCCCC/C=C\C/C=C\CCCCC)OC(=O)CCCN(C)C] |

In some embodiments, the cationically ionizable lipid has the structure of Formula (XI):

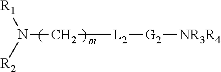

wherein
each of R¹ and R² is independently R₅ or -G₁-L₁-R₆, wherein at least one of R₁ and R₂ is -G₁-L₁-R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C—O)—, —(C—O)O—, —C(═O)—, —O—, —S(O)—, —S—S—, —C(═O)S—, —SC(═O)—, —NRaC(═O)—, —C(═O)NRa—, —NRaC(═O)NRa—, —OC(═O)NRa— and —NRaC(═O)O—;
Ra is H or C₁₋₁₂ alkyl;
m is 0, 1, 2, 3, or 4; and
x is 0, 1 or 2.

In some of the foregoing embodiments of Formula (XI), G₁ is independently unsubstituted C₁-C₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, each G₁ is independently unsubstituted C₆₋₁₂ alkylene or unsubstituted C₆₋₁₂ alkenylene, e.g., unsubstituted, straight C₆₋₁₂ alkylene or unsubstituted, straight C₆₋₁₂ alkenylene. In some embodiments, each G₁ is independently unsubstituted C₈₋₁₂ alkylene or unsubstituted C₈₋₁₂ alkenylene, e.g., unsubstituted, straight C₈₋₁₂ alkylene or unsubstituted, straight C₈₋₁₂ alkenylene. In some embodiments, each G₁ is independently unsubstituted C₆₋₁₀ alkylene or unsubstituted C₆₋₁₀ alkenylene, e.g., unsubstituted, straight C₆₋₁₀ alkylene or unsubstituted, straight C₆₋₁₀ alkenylene. In some embodiments, each G₁ is independently unsubstituted alkylene having 8, 9 or 10 carbon atoms, e.g., unsubstituted, straight alkylene having 8, 9 or 10 carbon atoms. In some embodiments, where R₁ and R₂ are both independently -G₁-L₁-R₆, G₁ for R₁ may be different from G₁ for R₂. In some of these embodiments, for example, G₁ for R₁ is unsubstituted, straight C₁₋₁₂ alkylene and G₁ for R₂ is unsubstituted, straight C₂₋₁₂ alkenylene; or G₁ for R₁ is an unsubstituted, straight C₁₋₁₂ alkylene group and G₁ for R₂ is a different unsubstituted, straight C₁₋₁₂ alkylene group. In some embodiments, where R₁ and R₂ are both independently -G₁-L₁-R₆, G₁ for R₁ may be identical to G₁ for R₂. In some of these embodiments, for example, each G₁ is the same unsubstituted, straight C₈₋₁₂ alkylene, such as unsubstituted, straight C₈₋₁₀ alkylene, or each G₁ is the same unsubstituted, straight C₆₋₁₂ alkenylene.

In some of the foregoing embodiments of Formula (XI), each L₁ is independently selected from the group consisting of —O(C═O)—, —(C═O)O—, —C(═O)S—, —SC(═O)—, —NRaC(═O)—, and —C(═O)NRa—. In some embodiments, Rₐ of L₁ is H or C₁₋₁₂ alkyl. In some embodiments, Rₐ of L₁ is H or C₁₋₆ alkyl, e.g., H or C₁₋₃ alkyl. In some embodiments. Ra of L₁ is H, methyl, or ethyl. In some embodiments, each L₁ is independently selected from the group consisting of —O(C═O)—, —(C═O)O—, —C(═O)S—, and —SC(═O)—. In some embodiments, each L₁ is independently —O(C═O)— or —(C═O)O—. In some embodiments, where R₁ and R₂ are both independently -G₁-L₁-R₆. L₁ for R₁ may be different from L₁ for R₂. In some of these embodiments, for example, L₁ for R₁ is one moiety selected from the group consisting of —O(C═O)—, —(C═O)O—, —C(═O)S—, —SC(═O)—, —NRaC(═O)—, and —C(═O)NRa— (e.g., L₁ for R₁ is —O(C═O)—), and L₁ for R₂ is a different moiety selected from the group consisting of —O(C═O)—, —(C═O)O—, —C(═O)S—, —SC(═O)—, —NRaC(═O)—, and —C(═O)NRa— (e.g., L₁ for R₂ is —(C═O)O—). In some embodiments, where R₁ and R₂ are both independently -G₁-L₁-R₆, L₁ for R₁ may be identical to L₁ for R₂. In some of these embodiments, for example, each L₁ is the same moiety selected from the group consisting of —O(C═O)—, —(C═O)O—, —C(═O)S—, —SC(═O)—, —NRaC(═O)—, and —C(═O)NRa—, e.g., each L₁ is —O(C═O)— or each L₁ is —(C═O)O—.

In some of the foregoing embodiments of Formula (XI), each R₆ is independently a non-cyclic hydrocarbyl group having at least 10 carbon atoms, e.g., a straight hydrocarbyl group having at least 10 carbon atoms. In some embodiments, each R₆ has independently at most 30 carbon atoms, such as at most 28, at most 26, at most 24, at most 22, or at most 20 carbon atoms. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms). In some embodiments, each R₆ is attached to L₁ via an internal carbon atom of R₆. In some embodiments, each R₆ has independently at most 30 carbon atoms (such as at most 28, at most 26, at most 24, at most 22, or at most 20 carbon atoms), and each R₆ is attached to L₁ via an internal carbon atom of R₆. In some embodiments, each R₆ is independently a non-cyclic hydrocarbyl group having at least 10 carbon atoms, e.g., a straight hydrocarbyl group having at least 10 carbon atoms, and each R₆ is attached to L₁ via an internal carbon atom of R₆. In some embodiments, each R₆ is independently a non-cyclic hydrocarbyl group having 10 to 30 carbon atoms (such as 10 to 28, 10 to 26, 10 to 24, 10 to 22, or 10 to 20 carbon atoms), e.g., a straight hydrocarbyl group having 10 to 30 carbon atoms (such as 10 to 28, 10 to 26, 10 to 24, 10 to 22, or 10 to 20 carbon atoms), and each R₆ is attached to L₁ via an internal carbon atom of R₆. In some embodiments, the hydrocarbyl group of R₆ is an alkyl or alkenyl group, e.g., a C₁₀₋₃₀ alkyl or alkenyl group. Thus, in some embodiments, each R₆ is independently a non-cyclic alkyl group having at least 10 carbon atoms or a non-cyclic alkenyl group having at least 10 carbon atoms, e.g., a straight alkyl group having at least 10 carbon atoms or a straight alkenyl group having at least 10 carbon atoms. In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms). In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms). In some embodiments, each R₆ is independently a non-cyclic alkyl group having 10 to 30 carbon atoms (such as 10 to 28, 10 to 26, 10 to 24, 10 to 22, or 10 to 20 carbon atoms) or a non-cyclic alkenyl group having 10 to 30 carbon atoms (such as 10 to 28, 10 to 26, 10 to 24, 10 to 22, or 10 to 20 carbon atoms), e.g., a straight alkyl group having 10 to 30 carbon atoms (such as 10 to 28, 10 to 26, 10 to 24, 10 to 22, or 10 to 20 carbon atoms) or a straight alkenyl group having 10 to 30 carbon atoms (such as 10 to 28, 10 to 26, 10 to 24, 10 to 22, or 10 to 20 carbon atoms), and each R₆ is attached to L₁ via an internal carbon atom of R₆. In some embodiments, each R₆ is independently a non-cyclic alkyl group having 11 to 19 carbon atoms (such as 11, 13, 15, 17, or 17 carbon atoms), e.g., a straight alkyl group having 11 to 19 carbon atoms (such as 11, 13, 15, 17, or 17 carbon atoms), and each R₆ is attached to L₁ via an internal carbon atom of R₆. The expression “internal carbon atom” means that the carbon atom of R₆ by which Re is attached to L₁ is directly bonded to at least 2 other carbon atoms of R₆. For example, for the following Cu alkyl group, each carbon atom at any one of positions 2, 3, 4, 5, and 7 qualifies as “internal carbon atom” according to the present disclosure, whereas the carbon atoms at positions 1, 6, 8, 9, 10, and 11 do not.

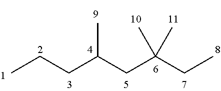

Consequently, R₆ being a C₁₁ alkyl group attached to L₁ via an internal carbon of R₆ includes the following groups:

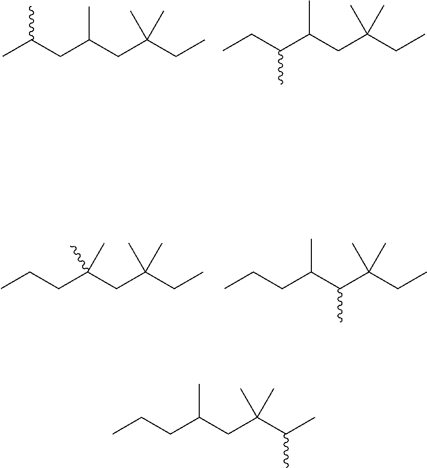

wherein [symbol] represents the bond by which R₆ is bound to L₁. Furthermore, for a straight alkyl group, e.g., a straight Cn alkyl group, each carbon atom except for the first and last carbon atoms of the straight alkyl group (i.e., except the carbon atoms at positions 1 and 11 of the straight Cu alkyl group) qualifies as “internal carbon atom”. Thus, in some embodiments, R₆ being a straight alkyl group having p carbon atoms and being attached to L₁ via an internal carbon atom of R₆ means that R₆ is attached to L₁ via a carbon atom of R₆ at any one of positions 2 to (p-1) (thereby excluding the terminal C atoms at positions 1 and p). In some embodiments, where R₆ is a straight alkyl group having p′ carbon atoms (wherein p′ is an even number) and being attached to L₁ via an internal carbon atom of R₆, R₆ is attached to L₁ via a carbon at any one of positions (p′/2−1), (p′/2), and (p′/2+1) of R₆ (e.g., if p′ is 10, R₆ is attached to L₁ via a carbon atom at any one of positions 4, 5, and 6 of R₆). In some embodiments, where R₆ is a straight alkyl group having p″ carbon atoms (wherein p″ is an uneven number) and being attached to L₁ via an internal carbon atom of R₆, R₆ is attached to L₁ via a carbon atom at any one of positions (p″−1)/2 and (p″+1)/2 of R₆ (e.g., if p″ is 11, R₆ is attached to L₁ via a carbon at any one of positions 5 and 6 of R₆). Generally, it is to be understood that if both R₁ and R₂ are -G₁-L₁-R₆ and each R₆ is attached to L₁ via an internal carbon atom of R₆, R₆ of R₁ is attached to L₁ of R₁ (and not to L₁ of R₂) via an internal carbon atom of R₆ of R₁ and R₆ of R₂ is attached to L₁ of R₂ (and not to L₁ of R₁) via an internal carbon atom of R₆ of R₂. In some embodiments, each R₆ is independently selected from the group consisting of:

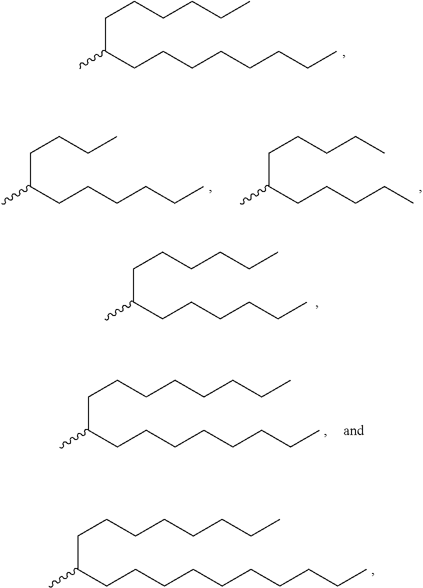

wherein [symbol] represents the bond by which R₆ is bound to L₁. In some embodiments, where R₁ and R₂ are both independently -G₁-L₁-R₆, R₆ for R₁ is different from R₆ for R₂. In some of these embodiments, for example, R₆ for R₁ may be a non-cyclic, preferably straight, hydrocarbyl group having at least 10 carbon atoms (e.g., R₆ for R₁ is

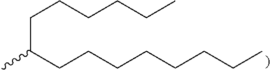 [SMILES: CCCCCCCCC(C)CCCCCC]

and R₆ for R₂ may be a different non-cyclic, preferably straight, hydrocarbyl group having at least 10 carbon atoms (e.g., R₆ for R₂ is

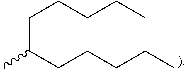

In some embodiments, where R₁ and R₂ are both independently -G₁-L₁-R₆, R₆ for R₁ is identical to R₆ for R₂. In some of these embodiments, for example, each R₆ is the same non-cyclic, preferably straight, hydrocarbyl group having at least 10 carbon atoms (e.g., each R₆ is

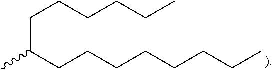 [SMILES: CCCCCCCCC(C)CCCCCC]

In some of the foregoing embodiments of Formula (XI), R₅ is a non-cyclic hydrocarbyl group having at least 10 carbon atoms, e.g., a straight hydrocarbyl group having at least 10 carbon atoms. In some embodiments, R₅ is a non-cyclic hydrocarbyl group having at least 12 carbon atoms, such as at least 14, at least 16, or at least 18 carbon atoms, e.g., a straight hydrocarbyl group having at least 12, at least 14, at least 16, or at least 18 carbon atoms. In some embodiments, R₅ has at most 30 carbon atoms, such as at most 28, at most 26, at most 24, at most 22, or at most 20 carbon atoms. In some embodiments, R₅ is a non-cyclic hydrocarbyl group, e.g., a straight hydrocarbyl group, wherein each hydrocarbyl group has 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the hydrocarbyl group of R₅ is an alkyl or alkenyl group, e.g., a C₁₀-30 alkyl or alkenyl group. Thus, in some embodiments, R₅ is a non-cyclic alkyl group having at least 10 carbon atoms (such as at least 12, at least 14, at least 16, or at least 18 carbon atoms) or a non-cyclic alkenyl group having at least 10 carbon atoms (such as at least 12, at least 14, at least 16, or at least 18 carbon atoms), e.g., a straight alkyl group having at least 10 carbon atoms (such as at least 12, at least 14, at least 16, or at least 18 carbon atoms) or a straight alkenyl group having at least 10 carbon atoms (such as at least 12, at least 14, at least 16, or at least 18 carbon atoms). In some embodiments. R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments, the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments, R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments. R₅ is a non-cyclic alkyl group or a non-cyclic alkenyl group, e.g., a straight alkyl group or a straight alkenyl group, wherein each of the alkyl and alkenyl groups has independently 10 to 30 carbon atoms (such as 10 to 28, 10 to 26, 10 to 24, 10 to 22, 10 to 20 carbon atoms, or 12 to 30, 12 to 28, 12 to 26, 12 to 24, 12 to 22, 12 to 20 carbon atoms, or 14 to 30, 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments, R₅ has the following structure:

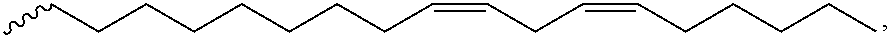

wherein [symbol] represents the bond by which R₅ is bound to the remainder of the compound.
In some of the foregoing embodiments of Formula (XI), L₂ is selected from the group consisting of —O(C═O)—, —(C═O)O—, —C(═O)—, —S—S—, —C(═O)S—, —SC(═O)—, —NRaC(═O)—, —C(═O)NRa—, —NRaC(═O)NRa—, —OC(═O)NRa— and —NRaC(═O)O—. In some embodiments, L₂ is selected from the group consisting of —O(C═O)—, —(C═O)O—, —C(═O)—, —C(═O)S—, —SC(═O)—, —NRaC(═O)—, and —C(═O)NRa—. In some embodiments, Rₐ of L₂ is H or C₁₋₁₂ alkyl. In some embodiments, Ra of L₂ is H or C₁₋₆ alkyl, e.g., H or C₁₋₃ alkyl. In some embodiments, Ra of L₂ is H, methyl, or ethyl. In some embodiments, L₂ is selected from the group consisting of —O(C═O)—, —(C═O)O—, —C(═O)S—, and —SC(═O)—. In some embodiments, L₂ is —O(C═O)— or —(C═O)O—.

In some of the foregoing embodiments of Formula (XI), G₂ is unsubstituted C₁₋₁₂ alkylene or unsubstituted C₂₋₁₂ alkenylene, e.g., unsubstituted, straight C₁₋₁₂ alkylene or unsubstituted, straight C₂₋₁₂ alkenylene. In some embodiments, G₂ is unsubstituted C₂₋₁₀ alkylene or unsubstituted C₂₋₁₀ alkenylene, e.g., unsubstituted, straight C₂₋₁₀ alkylene or unsubstituted, straight C₂₋₁₀ alkenylene. In some embodiments, G₂ is unsubstituted C₂₋₆ alkylene or unsubstituted C₂₋₆ alkenylene, e.g., unsubstituted, straight C₂₋₆ alkylene or unsubstituted, straight C₂₋₆ alkenylene. In some embodiments, G₂ is unsubstituted C₂₋₄ alkylene or unsubstituted C₂₋₄ alkenylene, e.g., unsubstituted, straight C₂₋₄ alkylene or unsubstituted, straight C₂₋₄ alkenylene. In some embodiments, G₂ is ethylene or trimethylene.

In some of the foregoing embodiments of Formula (XI), each of R₃ and R₄ is independently C₁₋₆ alkyl or C₂₋₆ alkenyl. In some embodiments, each of R₃ and R₄ is independently C₁₋₄ alkyl or C₂₋₄ alkenyl. In some embodiments, each of R₃ and R₄ is independently C₁₋₃ alkyl. In some embodiments, each of R₃ and R₄ is independently methyl or ethyl. In some embodiments, each of R₃ and R₄ is methyl.

In some of the foregoing embodiments of Formula (XI), m is 0, 1, 2 or 3. In some embodiments, m is 0 or 2. In some embodiments, m is 0. In some embodiments, m is 2.

In some of the foregoing embodiments of Formula (XI), the cationically ionizable lipid has the structure of Formula (XIIa) or (XIIb):

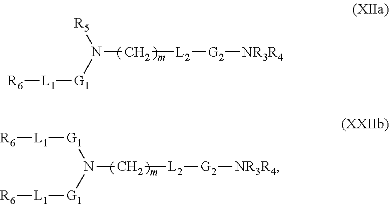

wherein
each of R₃ and R₄ is independently C₁-C₆ alkyl or C₂₋₆ alkenyl;
R₅ is a straight hydrocarbyl group having at least 14 carbon atoms (such as at least 16 carbon atoms), wherein the hydrocarbyl group preferably has at least 2 carbon-carbon double bonds;
each R₆ is independently a straight hydrocarbyl group (e.g., a straight alkyl group) having at least 10 carbon atoms and/or each R₆ is attached to L₁ via an internal carbon atom of R₆, preferably each R₆ is independently a straight hydrocarbyl group (e.g., a straight alkyl group) having at least 10 carbon atoms and each R₆ is attached to L₁ via an internal carbon atom of R₆;
each G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene, such as unsubstituted, straight C₈₋₁₂ alkylene or unsubstituted, straight C₈₋₁₂ alkenylene;
G₂ is unsubstituted C₂-C₁₀ alkylene or C₂₋₁₀ alkenylene, preferably unsubstituted C₂-C₆ alkylene or C₂₋₆ alkenylene;
each of L₁ and L₂ is independently —O(C═O)— or —(C═O)O—; and
m is 0, 1, 2 or 3, preferably 0 or 2.

In some of the foregoing embodiments of Formula (XIIa), R₅ has at most 30 carbon atoms, such as at most 28, at most 26, at most 24, at most 22, or at most 20 carbon atoms. In some embodiments of formulas (XIIa), R₅ is a straight hydrocarbyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), R₅ is a straight alkyl or alkenyl group having 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIa), R₅ is a straight alkyl group or a straight alkenyl group, wherein each of the alkyl and alkenyl groups has independently 14 to 30 carbon atoms (such as 14 to 28, 14 to 26, 14 to 24, 14 to 22, 14 to 20 carbon atoms, or 16 to 30, 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIa), R₅ has the following structure:

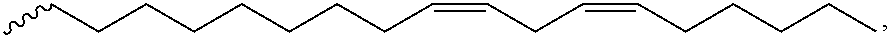

wherein [symbol] represents the bond by which R₅ is bound to the remainder of the compound. In some embodiments of formula (XIIa), R₆ has at most 30 carbon atoms, such as at most 28, at most 26, at most 24, at most 22, or at most 20 carbon atoms. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms). In some embodiments of formula (XIIa), R₆ is a straight hydrocarbyl group (e.g., a straight alkyl group) having at least 10 carbon atoms and R₆ is attached to L₁ via an internal carbon atom of R₆. In some embodiments of formula (XIIa), R₆ is a non-cyclic hydrocarbyl group (e.g., a non-cyclic alkyl group) having 10 to 30 carbon atoms (such as 10 to 28, 10 to 26, 10 to 24, 10 to 22, or 10 to 20 carbon atoms), e.g., a straight hydrocarbyl group (e.g., a straight alkyl group) having 10 to 30 carbon atoms (such as 10 to 28, 10 to 26, 10 to 24, 10 to 22, or 10 to 20 carbon atoms), and R₆ is attached to L₁ via an internal carbon atom of R₆. In some embodiments of formula (XIIa), G₁ is independently unsubstituted, straight C₄-12 alkylene or C₄-12 alkenylene, e.g., unsubstituted, straight C₆-12 alkylene or C₆-12 alkenylene. In some embodiments of formula (XIIa), R₅ is a straight hydrocarbyl group, e.g., a straight alkenyl group, having at least 14 carbon atoms (such as 14 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L₁ via an internal carbon atom of R₆; and G₁ is independently unsubstituted, straight C₄-12 alkylene or C₄-12 alkenylene, e.g., unsubstituted, straight C₆-12 alkylene or C₆-12 alkenylene.

In some of the foregoing embodiments of Formula (XIIb), each R₆ has independently at most 30 carbon atoms, such as at most 28, at most 26, at most 24, at most 22, or at most 20 carbon atoms. In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms). In some embodiments of formula (XIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIb), each R₆ is independently selected from the group consisting of:

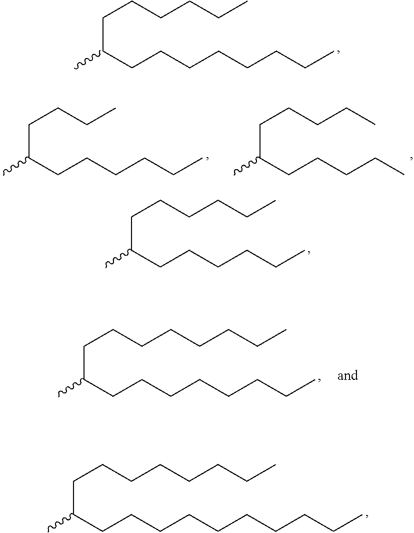

wherein [symbol] represents the bond by which R₆ is bound to L₁. In some embodiments of formula (XIIb), each G₁ is independently unsubstituted, straight C₆-12 alkylene or C₆-12 alkenylene. In some embodiments of formula (XIIb), each G₁ is independently unsubstituted, straight C₈₋₁₂ alkylene or C₈₋₁₂ alkenylene. In some embodiments of formula (XIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G₁ is independently unsubstituted, straight C₈₋₁₂ alkylene or C₈₋₁₂ alkenylene.

In some of the foregoing embodiments of Formula (XI), the cationically ionizable lipid has the structure of Formula (XIIIa) or (XIIIb):

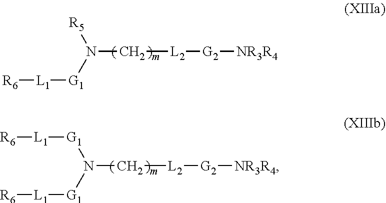

wherein
each of R₃ and R₄ is independently C₁₋₄ alkyl or C₂₋₄ alkenyl, more preferably C₁₋₃ alkyl, such as methyl or ethyl;
R₅ is a straight alkyl or alkenyl group having at least 16 carbon atoms, wherein the alkenyl group preferably has at least 2 carbon-carbon double bonds;
each R₆ is independently a straight hydrocarbyl group having at least 10 carbon atoms, wherein R₆ is attached to L₁ via an internal carbon atom of R₆;
each G₁ is independently unsubstituted, straight C₆-12 alkylene or unsubstituted, straight C₆-12 alkenylene, e.g., unsubstituted, straight C₈₋₁₂ alkylene or unsubstituted, straight C₈₋₁₂ alkenylene, such as unsubstituted, straight C₈₋₁₀ alkylene or unsubstituted, straight C₈₋₁₀ alkenylene, such as unsubstituted, straight C₈ alkylene;
G₂ is unsubstituted C₂₋₆ alkylene or C₂₋₆ alkenylene, preferably unsubstituted C₂₋₄ alkylene or C₂₋₄ alkenylene, such as ethylene or trimethylene;
each of L₁ and L₂ is independently —O(C═O)— or —(C═O)O—; and
m is 0, 1, 2 or 3, preferably 0 or 2.

In some of the foregoing embodiments of Formula (XIIIa), R₅ has at most 30 carbon atoms, such as at most 28, at most 26, at most 24, at most 22, or at most 20 carbon atoms. In some embodiments of formulas (XIIIa), R₅ is a straight alkyl or alkenyl group having 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms). In some embodiments of formula (XIIIa), the alkenyl group has at least 2 carbon-carbon double bonds, e.g., 2 or 3 carbon-carbon double bonds, such as 2 carbon-carbon double bonds. In some embodiments, the alkenyl group has at least 1 carbon-carbon double bond in cis configuration, e.g., 1, 2 or 3, such as 2, carbon-carbon double bonds in cis configuration. Thus, in some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 2 carbon-carbon double bonds, e.g., 2 or 3 carbon-carbon double bonds. In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has at least 1 carbon-carbon double bond, such as 1, 2, or 3 carbon-carbon double bonds, in cis configuration. In some embodiments of formula (XIIIa), R₅ is a straight alkyl group or a straight alkenyl group, wherein each of the alkyl and alkenyl groups has independently 16 to 30 carbon atoms (such as 16 to 28, 16 to 26, 16 to 24, 16 to 22, 16 to 20 carbon atoms, or 18 to 30, 18 to 28, 18 to 26, 18 to 24, 18 to 22, or 18 to 20 carbon atoms) and the alkenyl group has 2 or 3 carbon-carbon double bonds, wherein at least 1 carbon-carbon double bond, such as 1, 2, or 3 carbon-carbon double bonds, is in cis configuration. In some embodiments of formula (XIIIa), R₅ has the following structure:

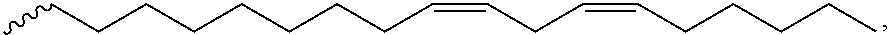

wherein [symbol] represents the bond by which R₅ is bound to the remainder of the compound. In some embodiments of formula (XIIIa), R₆ has at most 30 carbon atoms, such as at most 28, at most 26, at most 24, at most 22, or at most 20 carbon atoms. In some embodiments of formula (XIIIa), R₆ is a straight hydrocarbyl group (e.g., a straight alkyl group) having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), R₆ is a straight alkyl group having 10 to 30 carbon atoms (such as 10 to 28, 10 to 26, 10 to 24, 10 to 22, or 10 to 20 carbon atoms) and R₆ is attached to L₁ via an internal carbon atom of R₆. In some embodiments of formula (XIIIa), G₁ is independently unsubstituted, straight C₄₋₁₂ alkylene or C₄₋₁₂ alkenylene, e.g., unsubstituted, straight C₆₋₁₂ alkylene or C₆₋₁₂ alkenylene. In some embodiments of formula (XIIIa), R₅ is a straight hydrocarbyl group, e.g., a straight alkenyl group, having at least 16 carbon atoms (such as 16 to 30 carbon atoms) and 2 or 3 carbon-carbon double bonds; R₆ is a straight hydrocarbyl group (e.g., a straight alkyl group) having at least 10 carbon atoms (e.g., having 10 to 30 carbon atoms) and R₆ is attached to L₁ via an internal carbon atom of R₆; and G₁ is independently unsubstituted, straight C₄-12 alkylene or C₄-12 alkenylene, e.g., unsubstituted, straight C₆-12 alkylene or C₆-12 alkenylene.

In some of the foregoing embodiments of Formula (XIIIb), each R₆ has independently at most 30 carbon atoms, such as at most 28, at most 26, at most 24, at most 22, or at most 20 carbon atoms. In some embodiments of formula (XIIIb), each R₆ is independently a straight hydrocarbyl group (e.g., a straight alkyl group) having 10 to 30 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and each R₆ is attached to L₁ via an internal carbon atom of R₆. In some embodiments of formula (XIIIb), each R₆ is attached to L₁ via an internal carbon atom of R₆ and is independently selected from the group consisting of:

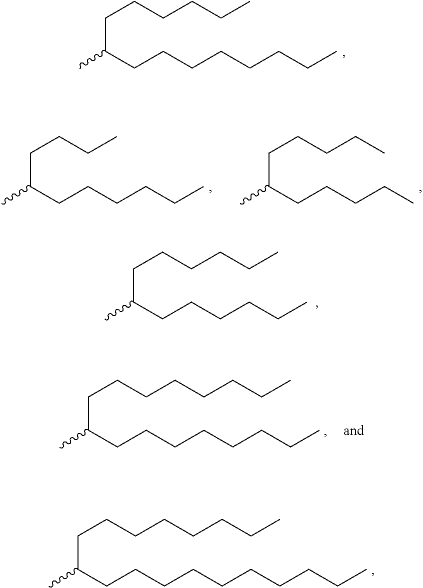

wherein [symbol] represents the bond by which R₆ is bound to L₁. In some embodiments of formula (XIIIb), each G₁ is independently unsubstituted, straight C₈₋₁₂ alkylene or C₈₋₁₂ alkenylene, e.g., unsubstituted, straight C₈₋₁₀ alkylene or C₈₋₁₀ alkenylene. In some embodiments of formula (XIIIb), each R₆ is independently a straight hydrocarbyl group (e.g., a straight alkyl group) having at least 10 carbon atoms (such as 10 to 28, 10 to 26, 10 to 24, 10 to 22, or 10 to 20 carbon atoms, or 11 to 19 carbon atoms, such as 11, 13, 15, 17, or 17 carbon atoms) and is attached to L₁ via an internal carbon atom of R₆; and each G₁ is independently unsubstituted, straight C₈₋₁₂ alkylene or C₈₋₁₂ alkenylene, e.g., unsubstituted, straight C₈₋₁₀ alkylene or C₈₋₁₀ alkenylene.

In some of the foregoing embodiments of Formula (XI), the cationically ionizable lipid has one of the following formulas (XIV-1), (XIV-2), and (XIV-3):

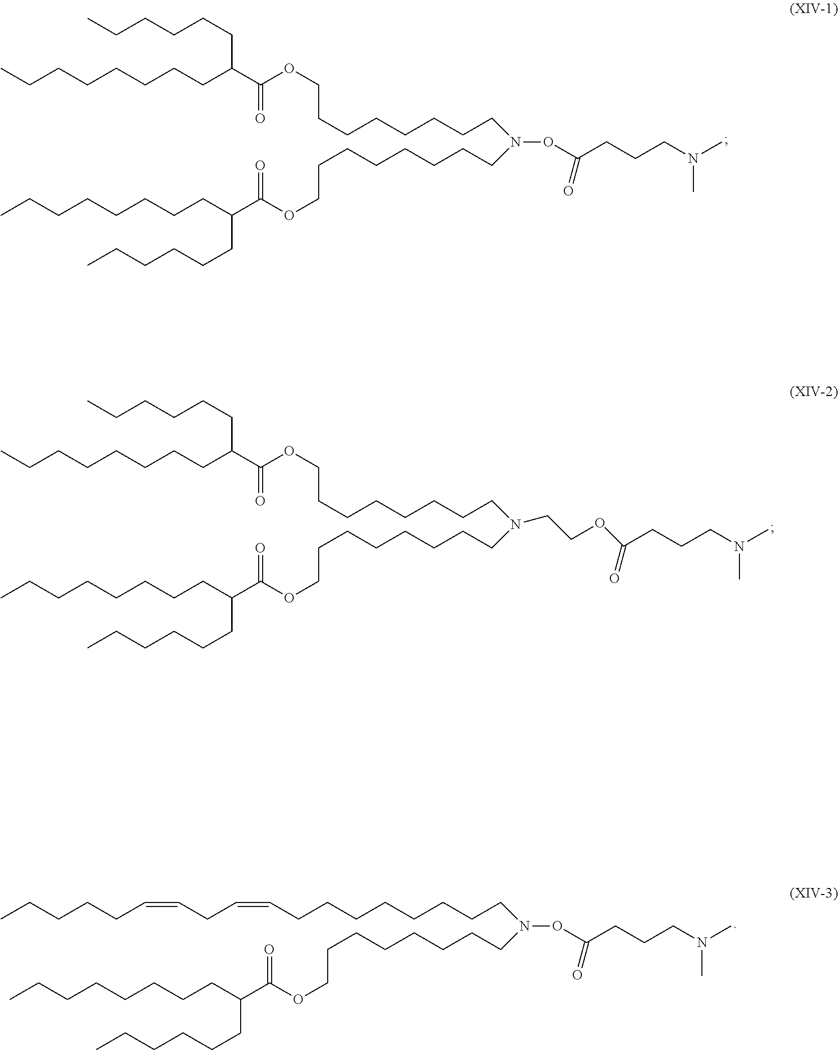

In some embodiments, the cationically ionizable lipid is (6Z,16Z)-12-((Z)-dec-4-en-1-yl) docosa-6,16-dien-11-yl 5-(dimethylamino) pentanoate (3D-P-DMA). The structure of 3D-P-DMA may be represented as follows:

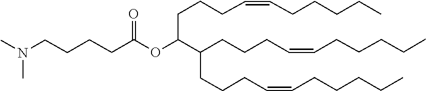 [SMILES: CCCCC/C=C\CCCC(CCC/C=C\CCCCC)C(CCC/C=C\CCCCC)OC(=O)CCCCN(C)C]

In various different embodiments, the cationically ionizable lipid is selected from the group consisting of N,N-dimethyl-2,3-dioleyloxypropylamine (DODMA), 1,2-dioleoyl-3-dimethylammonium-propane (DODAP), heptatriaconta-6,9,28,31-tetraen-19-yl-4-(dimethylamino) butanoate (DLin-MC3-DMA), and 4-((di((9Z,12Z)-octadeca-9,12-dien-1-yl)amino)oxy)-N,N-dimethyl-4-oxobutan-1-amine (DPL-14).

Further examples of cationically ionizable lipids include, but are not limited to, 3-(N—(N′,N′-dimethylaminoethane)-carbamoyl) cholesterol (DC-Chol), 1,2-dioleoyl-3-dimethylammonium-propane (DODAP); 1,2-diacyloxy-3-dimethylammonium propanes; 1,2-dialkyloxy-3-dimethylammonium propanes, 1,2-distearyloxy-N,N-dimethyl-3-aminopropane (DSDMA), 1,2-dilinoleyloxy-N,N-dimethylaminopropane (DLinDMA), 1,2-dilinolenyloxy-N,N-dimethylaminopropane (DLenDMA), dioctadecylamidoglycyl spermine (DOGS), 3-dimethylamino-2-(cholest-5-en-3-beta-oxybutan-4-oxy)-1-(cis,cis-9,12-oc-tadecadienoxy) propane (CLinDMA), 2-[5′-(cholest-5-en-3-beta-oxy)-3′-oxapentoxy)-3-dimethyl-1-(cis,cis-9′,12′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-K-DMA), 2,2-dilinoleyl-4-dimethylaminoethyl-[1,3]-dioxolane (DLin-K-XTC2-DMA), 2,2-dilinoleyl-4-(2-dimethylaminoethyl)-[1,3]-dioxolane (DLin-KC2-DMA), heptatriaconta-6,9,28,31-tetraen-19-yl-4-(dimethylamino) butanoate (DLin-MC3-DMA), 2-({8-[(3)-cholest-5-en-3-yloxy]octyl}oxy)-N,N-dimethyl-3-[(9Z,12Z)-octadeca-9,12-dien-1-yloxy]propan-1-amine (Octyl-CLinDMA), 1,2-dimyristoyl-3-dimethylammonium-propane (DMDAP), 1,2-dipalmitoyl-3-dimethylammonium-propane (DPDAP), N1-[2-((1S)-1-[(3-aminopropyl)amino]-4-[di(3-aminopropyl)amino]butylcarboxamido)ethyl]-3,4-di[oleyloxy]-benzamide (MVL5), di((Z)-non-2-en-1-yl) 8,8′-((((2 (dimethylamino)ethyl)thio) carbonyl) azanediyl)dioctanoate (ATX), N,N-dimethyl-2,3-bis(dodecyloxy) propan-1-amine (DLDMA), N,N-dimethyl-2,3-bis(tetradecyloxy) propan-1-amine (DMDMA), di((Z)-non-2-en-1-yl)-9-((4-(dimethylaminobutanoyl)oxy) heptadecanedioate (L₃₁₉), N-dodecyl-3-((2-dodecylcarbamoyl-ethyl)-{2-[(2-dodecylcarbamoyl-ethyl)-2-{(2-dodecylcarbamoyl-ethyl)-[2-(2-dodecylcarbamoyl-ethylamino)-ethyl]-amino}-ethylamino) propionamide (lipidoid 98N12-5), 1-[2-[bis(2-hydroxydodecyl)amino]ethyl-[2-[4-[2-[bis(2 hydroxydodecyl)amino]ethyl]piperazin-1-yl]ethyl]amino]dodecan-2-ol (lipidoid C12-200).

In certain embodiments, the cationically ionizable lipid has the structure X-3.

In some embodiments, the cationic lipid for use herein is or comprises DPL14. As used herein, “DPL14” is a lipid comprising the following general formula:

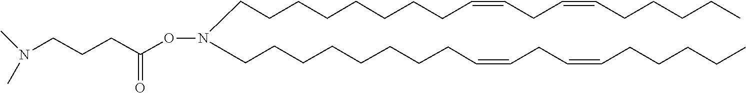

In some embodiments, the cationically ionizable lipid comprises from about 10 mol % to about 80 mol %, such as from about 20 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipid present in the compositions (such as of the total lipid present in the particles, in particular, of the total lipid and lipid-like material present in the LNPs).

Conjugate of a POX and/or POZ Polymer and One or More Hydrophobic Chains

One or more of the particle-forming components described herein such as polymers, lipids or lipid-like materials used in the compositions and particles described herein comprise a polyoxazoline (POX) and/or polyoxazine (POZ) polymer, such as in the conjugates of the present disclosure which comprises (i) a polyoxazoline (POX) and/or polyoxazine (POZ) polymer and (ii) one or more hydrophobic chains as disclosed herein.

It has been found that the pharmacology of encapsulated nucleic acid (in particular RNA) can be controlled in a predictable manner by the following modifications: (a) modulating the length of the hydrophobic chain(s) of the conjugate; and/or (b) increasing the hydrophilicity at the intersection between the hydrophobic chain(s) and the POX and/or POZ polymer of the conjugate, in particular, by introducing a functional moiety at this intersection (e.g., by inserting, between the hydrophobic chain(s) and the POX and/or POZ polymer of the conjugate, (i) an alkylene moiety substituted with at least one monovalent functional moiety and/or (ii) an alkylene moiety linked to a divalent functional moiety such that the divalent functional moiety links the hydrophobic chain(s) to the alkylene moiety which in turn is attached to the POX and/or POZ polymer. Without wishing to be bound by any theory, it is assumed that the introduction of such functional moiety at the intersection between the hydrophobic chain(s) and the POX and/or POZ polymer of the conjugate results in the attraction of water molecules to the intersection which, in turn, weakens the interaction of the hydrophobic chain(s) with the particle (probably because the hydrophobic chain(s) are, to some extent, pulled out of the particle). A similar result can be obtained by shortening the length of the hydrophobic chain(s) which also results in a weaker interaction of the hydrophobic chain(s) with the particle. Thus, in a vial, i.e., prior to administration, the nucleic acid (e.g., DNA or RNA) particles retain a full complement of the conjugate disclosed herein (thereby preventing the aggregation of individual nucleic acid (e.g., DNA or RNA) particles). However, in the blood compartment, i.e., after administration, the conjugate dissociates from the nucleic acid (e.g., DNA or RNA) particle over time, revealing a more fusogenic particle that is more readily taken up by cells, ultimately leading to release of the nucleic acid (e.g., DNA or RNA) payload.

In some embodiments, in the conjugate comprising (i) a POX and/or POZ polymer and (ii) one or more hydrophobic chains, components (i) and (ii) are linked to each other via a linker which comprises at least one functional moiety. In some embodiments, said linker comprises an alkylene moiety substituted with at least one monovalent functional moiety. In some embodiments, said linker comprises an alkylene group and a divalent functional moiety, wherein the divalent functional moiety links the alkylene group to the one or more hydrophobic chains, and the alkylene group is attached to the POX and/or POZ polymer. In some embodiments, said linker comprises an alkylene group and a divalent functional moiety, wherein the divalent functional moiety links the alkylene group to the one or more hydrophobic chains, the alkylene group is substituted with at least one monovalent functional moiety, and the alkylene group is attached to the POX and/or POZ polymer.

In some embodiments, each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties.

In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

In some embodiments, the conjugate comprises one of the following structures (in particular, if the one or more hydrophobic chains are attached to the N-end (i.e., the terminal N atom) of the POX and/or POZ polymer, as shown, for example in formula (II) herein):
(hydrophobic chain)₁₋₂-(alkylene moiety substituted with at least one monovalent functional moiety)-(POX and/or POZ polymer)
[(hydrophobic chain)-(divalent functional moiety)]₁₋₂-(alkylene moiety)-(POX and/or POZ polymer).

In some embodiments, the conjugate has one of the following formulas (in particular, if the one or more hydrophobic chains are attached to the N-end (i.e., the terminal N atom) of the POX and/or POZ polymer, as shown, for example in formula (II) herein below):
(hydrophobic chain)₁₋₂-(alkylene moiety substituted with at least one monovalent functional moiety)-(POX and/or POZ polymer)-(end group)
[(hydrophobic chain)-(divalent functional moiety)]₁₋₂-(alkylene moiety)-(POX and/or POZ polymer)-(end group).

In some embodiments, the conjugate comprises the following structure (in particular, if the one or more hydrophobic chains are attached to the N-end (i.e., the terminal N atom) of the POX and/or POZ polymer, as shown, for example in formula (II) herein): [(hydrophobic chain) 2-(trivalent functional moiety)]-(alkylene moiety)-(POX and/or POZ polymer).

Thus, in some embodiments, the conjugate has the following formula (in particular, if the one or more hydrophobic chains are attached to the N-end (i.e., the terminal N atom) of the POX and/or POZ polymer, as shown, for example in formula (II) herein below):
[(hydrophobic chain)₂-(trivalent functional moiety)]-(alkylene moiety)-(POX and/or POZ polymer)-(end group).

In some embodiments, the end group is a neutral end group (such as H, alkyl, alkoxy, ester, or amide end group) or a functionalized end group (e.g., hydroxy, thiol, cyano, azido, or amino end group). In some embodiments, the end group is R³ as defined herein (in particular with respect to any one of formulas (II), (II′), (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (VI), (VI′), (VIa), (VIa′), (VII), (VII′), (VIIa), (VIIa′), (VIII), (VIII′), (VIIIa), (IX), (IX′), and (IXa), or to any one of formulas (IIe), (XV), (XV′), (XVI), (XVII), (XVII′), (XVIII), (XVIII′), (XIX), (XIX′), (XX′), (XXI′), (XXII′), and (XXIII′)) or R⁶ as defined herein (in particular with respect to any one of formulas (IV), (IV′), (IVa), (IVb), (IVc′), (IVd′), (IVe′), (IVf), (IVg′), (IVh′), (V), and (V′) or to formula (IVc)).

In some embodiments, the alkylene moiety substituted with at least one monovalent functional moiety is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments, the alkylene moiety is C₁₋₆-alkylene, such as C₁₋₃-alkylene, e.g., methylene, ethylene, or trimethylene.

In some embodiments (in particular those, where the one or more hydrophobic chains are attached to the C-end (i.e., the terminal C atom) of the POX and/or POZ polymer, as shown, for example in formula (II′) herein), the linker comprises at least one difunctional moiety via which the one or more hydrophobic chains are attached to the POX and/or POZ polymer. In some embodiments, the linker may additionally comprise an alkylene moiety (such as a C₁₋₆ alkylene moiety, e.g., a C₁₋₃ alkylene moiety), a cycloalkylene moiety (preferably a C₃₋₈-cycloalkylene, such as C₃₋₆-cycloalkylene moiety), or a cycloalkenylene moiety (preferably a C₃₋₈-cycloalkenylene, such as C₃₋₆-cycloalkenylene moiety) each of which connects the difunctional moiety to the POX and/or POZ polymer (either directly to the end of the POX and/or POZ polymer or, preferably, via a further difunctional moiety). For example, one hydrophobic chain may be attached to the end of the POX and/or POZ polymer via one difunctional moiety (either directly or via an alkylene, cycloalkylene, or cycloalkenylene moiety or via an alkylene, cycloalkylene, or cycloalkenylene moiety which bears another difunctional moiety); two hydrophobic chains may be attached to the end of the POX and/or POZ polymer via two difunctional moieties (which in turn are preferably attached to an alkylene, cycloalkylene, or cycloalkenylene moiety or to an alkylene, cycloalkylene, or cycloalkenylene moiety bearing another difunctional moiety); or two hydrophobic chains may be attached to the end of the POX and/or POZ polymer via the same difunctional moiety (which is then a trifunctional moiety and which may be attached to the end of the POX and/or POZ polymer either directly or via an alkylene, cycloalkylene, or cycloalkenylene moiety or to an alkylene, cycloalkylene, or cycloalkenylene moiety bearing another difunctional moiety). In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

In some embodiments, the cycloalkylene moiety is C₃₋₈-cycloalkylene, such as C₃₋₆-cycloalkylene, e.g., cyclopropylene, cyclobutylene, cyclopentylene, cyclohexylene, wherein the cycloalkylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments, the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein the cycloalkenylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments, the alkylene moiety is C₁₋₆-alkylene, such as C₁₋₃-alkylene, e.g., methylene, ethylene, or trimethylene, or C₂₋₃ alkylene.

In some embodiments (in particular those, where the one or more hydrophobic chains are attached to the C-end (i.e., the terminal C atom) of the POX and/or POZ polymer, as shown, for example in formula (II′) herein below), the conjugate comprises one of the following structures (and may have the general formula (II′)):
(hydrophobic chain)-(divalent functional moiety)-(POX and/or POZ polymer) [(hydrophobic chain)-(divalent functional moiety)]₁₋₂-(alkylene moiety)-(divalent functional moiety)-(POX and/or POZ polymer)
(hydrophobic chain)-(divalent functional moiety)-(cycloalkylene moiety)-(divalent functional moiety)-(POX and/or POZ polymer)
(hydrophobic chain)-(divalent functional moiety)-(cycloalkenylene moiety)-(divalent functional moiety)-(POX and/or POZ polymer)
(hydrophobic chain)-(divalent functional moiety)-(alkylene moiety)-(POX and/or POZ polymer)
[(hydrophobic chain)₂-(trivalent functional moiety)]-(alkylene moiety)-(divalent functional moiety)-(POX and/or POZ polymer)

In some embodiments (in particular those, where the one or more hydrophobic chains are attached to the C-end (i.e., the terminal C atom) of the POX and/or POZ polymer, as shown, for example in formula (II′) herein below), the conjugate has one of the following formulas (and may fall within general formula (II′)):
(hydrophobic chain)-(divalent functional moiety)-(POX and/or POZ polymer)-(end group) [(hydrophobic chain)-(divalent functional moiety)]₁₋₂-(alkylene moiety)-(divalent functional moiety)-(POX and/or POZ polymer)-(end group)
(hydrophobic chain)-(divalent functional moiety)-(cycloalkylene moiety)-(divalent functional moiety)-(POX and/or POZ polymer)-(end group)
(hydrophobic chain)-(divalent functional moiety)-(cycloalkenylene moiety)-(divalent functional moiety)-(POX and/or POZ polymer)-(end group)
(hydrophobic chain)-(divalent functional moiety)-(alkylene moiety)-(POX and/or POZ polymer)-(end group)
[(hydrophobic chain)₂-(trivalent functional moiety)]-(alkylene moiety)-(divalent functional moiety)-(POX and/or POZ polymer)-(end group)

The POX and/or POZ polymer may comprise a neutral end group (such as H, alkyl, alkoxy, ester, or amide end group) or a functionalized end group (e.g., hydroxy, thiol, cyano, azido, or amino end group). In some embodiments, the end group is R³ as defined herein (in particular with respect to any one of formulas (II), (II′), (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (VI), (VI′), (VIa), (VIa′), (VII), (VII′), (VIIa), (VIIa′), (VIII), (VIII′), (VIIIa), (IX), (IX′), and (IXa), or to any one of formulas (IIe), (XV), (XV′), (XVI), (XVII), (XVII′), (XVIII), (XVIII′), (XIX), (XIX′), (XX′), (XXI′), (XXII′), and (XXIII′)) or R⁶ as defined herein (in particular with respect to any one of formulas (IV), (IV′), (IVa), (IVb), (IVc′), (IVd′), (IVe′), (IVf), (IVg′), (IVh′), (V), and (V′), or to formula (IVc)).

In the case of nucleic acid (e.g., DNA or RNA)-lipid particles, the POX and/or POZ polymer is conjugated to, preferably covalently bound to one or more hydrophobic chains.

In certain embodiments, the end groups of the POX and/or POZ polymer may be functionalized with one or more molecular moieties conferring certain properties, such as positive or negative charge, or a targeting agent that will direct the particle to a particular cell type, collection of cells, or tissue.

A variety of suitable targeting agents are known in the art. Non-limiting examples of targeting agents include a peptide, a protein, an enzyme, a nucleic acid, a fatty acid, a hormone, an antibody, a carbohydrate, mono-, oligo- or polysaccharides, a peptidoglycan, a glycopeptide, or the like. In some embodiments, targeting agents include targeting pairs, such as the following pairs: antigen-antibody specific for said antigen; avidin-streptavidin; folate-folate receptor; transferrin-transferrin receptor; aptamer-molecule for which the aptamer is specific (e.g., pegaptanib-VEGF receptor); arginine-glycine-aspartic acid (RGD) peptide-αᵥβ₃ integrin; asparagine-glycine-arginine (NGR) peptide-aminopeptidase N; galactose-asialoglyco-protein receptor. For example, any of a number of different materials that bind to antigens on the surfaces of target cells can be employed. Antibodies to target cell surface antigens will generally exhibit the necessary specificity for the target. In addition to antibodies, suitable immunoreactive fragments can also be employed, such as the Fab, Fab′, F(ab′)2 or scFv fragments or single-domain antibodies (e.g. camelids V_{H}H fragments). Many antibody fragments suitable for use in forming the targeting mechanism are already available in the art. Similarly, ligands for any receptors on the surface of the target cells can suitably be employed as targeting agent. These include any small molecule or biomolecule, natural or synthetic, which binds specifically to a cell surface receptor, protein or glycoprotein found at the surface of the desired target cell.

In certain embodiments, the POX and/or POZ polymer comprises between 2 and 200, between 2 and 190, between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70 POX and/or POZ repeating units.

In certain embodiments, the POX and/or POZ polymer comprises the following general formula (I):

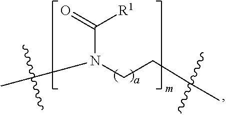

wherein a is an integer between 1 and 2; R¹ is alkyl, in particular C₁₋₃ alkyl, such as methyl, ethyl, iso-propyl, or n-propyl, and is independently selected for each repeating unit; and m refers to the number of POX and/or POZ repeating units.

In some embodiments, the POX and/or POZ polymer is a polymer of POX and comprises repeating units of the following general formula (Ia):

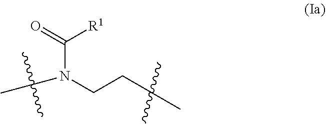

In some embodiments, the POX and/or POZ polymer is a polymer of POZ and comprises repeating units of the following general formula (Ib):

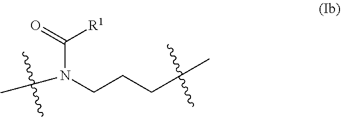

In any of the above embodiments of formulas (I), (Ia), and (Ib), m (i.e., the number of repeating units of formula (Ia) or formula (Ib) in the polymer) preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments of any of the above embodiments of formulas (I), (Ia), and (Ib), m is 2 to 180, such as 4 to 160, 6 to 140, 8 to 120 or 10 to 100, e.g., 20 to 80, 30 to 70, or 40 to 50.

In some embodiments, the POX and/or POZ polymer is a copolymer comprising repeating units of the following general formulas (la) and (Ib):

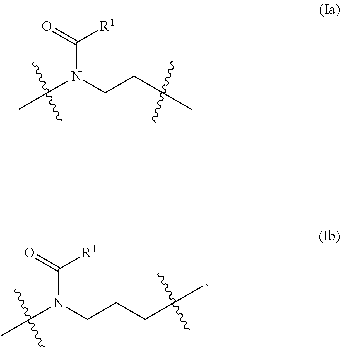

wherein the number of repeating units of formula (Ia) in the copolymer is 1 to 199; the number of repeating units of formula (Ib) in the copolymer is 1 to 199; and the sum of the number of repeating units of formula (Ia) and the number of repeating units of formula (Ib) in the copolymer is 2 to 200.

In some embodiments, the number of repeating units of formula (Ia) in the copolymer is 1 to 179, such as 1 to 159, 1 to 139, 1 to 119 or 1 to 99; the number of repeating units of formula (Ib) in the copolymer is 1 to 179, such as 1 to 159, 1 to 139, 1 to 119 or 1 to 99; and the sum of the number of repeating units of formula (Ia) and the number of repeating units of formula (Ib) in the copolymer is 2 to 180, such as 4 to 160, 6 to 140, 8 to 120 or 10 to 100, e.g., 20 to 80, 30 to 70, or 40 to 50.

In some of the above embodiments of formulas (I), (Ia), and (Ib), R¹ at each occurrence (i.e., in each repeating unit) may be the same alkyl group (e.g., R¹ may be methyl in each repeating unit). In some alternative embodiments of formulas (I), (Ia), and (Ib), R¹ in at least one repeating unit differs from R¹ in another repeating unit (e.g., for at least one repeating unit R¹ is one specific alkyl (such as ethyl), and for at least one different repeating unit R¹ is a different specific alkyl (such as methyl)). For example, each R¹ may be selected from two different alkyl groups (such as methyl and ethyl) and not all R¹ are the same alkyl.

In any of the above embodiments of formulas (I), (Ia), and (Ib), R¹ preferably is methyl or ethyl, more preferably methyl. Thus, in some embodiments of formulas (I), (Ia), and (Ib), each R¹ is methyl or each R¹ is ethyl. In some alternative embodiments of formulas (I), (Ia), and (Ib), R¹ is independently selected from methyl and ethyl for each repeating unit, wherein in at least one repeating unit R¹ is methyl, and in at least one repeating unit R¹ is ethyl.

In some embodiments, the conjugate has the following general formula (II) or (II′):

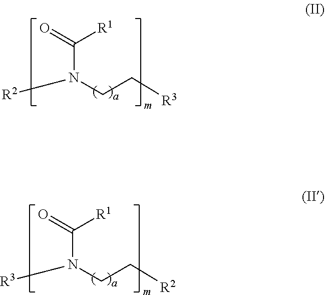

wherein:
a is an integer between 1 and 2;
R¹ is alkyl, in particular C₁₋₃ alkyl, such as methyl, ethyl, iso-propyl, or n-propyl, and is independently selected for each repeating unit;
m is 2 to 200;
R² is R⁴ or -L¹(R⁴)ₚ, wherein each R⁴ is independently a hydrocarbyl group; L¹ is a linker; and p is 1 or 2; and
R³ is selected from the group consisting of H, C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, and —NR²²C(O)R²¹, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more (such as 1, 2, 3, or 4) substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair. In formula (II) R² is attached to the N-end (i.e., the terminal N atom) of the POX and/or POZ polymer and R³ is attached to the C-end (i.e., the terminal C atom) of the POX and/or POZ polymer, whereas in formula (II′) R² is attached to the C-end (i.e., the terminal C atom) of the POX and/or POZ polymer and R³ is attached to the N-end (i.e., the terminal N atom) of the POX and/or POZ polymer.

In some embodiments, the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor. Thus, in some embodiments, a member of a targeting pair includes one of the following: an antigen, an antibody, avidin, streptavidin, folate, transferrin, an aptamer; an RGD peptide; an NGR peptide; and galactose.

In some embodiments of formula (II), a is 1, i.e., the conjugate has the following general formula (IIa) or (IIa′):

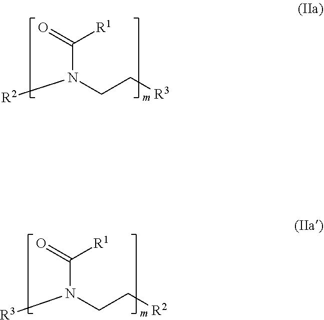

In some embodiments of formula (II), a is 2, i.e., the conjugate has the following general formula (IIb) or (IIb′):

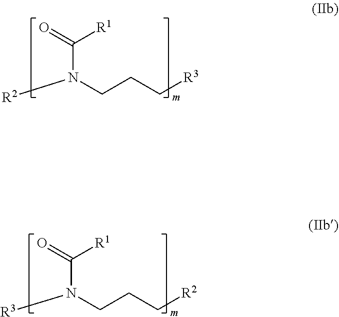

In any of the above embodiments of formulas (IIa), (IIa′), (IIb), and (IIb′), R¹, R², R³, and m are as defined for formula (II)/(II′).

In some of the above embodiments of formulas (II), (II′), (IIa), (IIa′), (IIb), and (IIb′), R¹ at each occurrence (i.e., in each repeating unit) may be the same alkyl group (e.g., R¹ may be methyl in each repeating unit). In some alternative embodiments of formulas (II), (II′), (IIa), (IIa′), (IIb), and (IIb′), R¹ in at least one repeating unit differs from R¹ in another repeating unit (e.g., for at least one repeating unit R¹ is one specific alkyl (such as ethyl), and for at least one different repeating unit R¹ is a different specific alkyl (such as methyl)). For example, each R¹ may be selected from two different alkyl groups (such as methyl and ethyl) and not all R¹ are the same alkyl.

In any of the above embodiments of formulas (II), (II′), (IIa), (IIa′), (IIb), and (IIb′), R¹ preferably is methyl or ethyl, more preferably methyl. Thus, in some embodiments of formulas (II), (II′), (IIa), (IIa′), (IIb), and (IIb′), each R¹ is methyl or each R¹ is ethyl. In some alternative embodiments of formulas (II), (II′), (IIa), (IIa′), (IIb), and (IIb′), R¹ is independently selected from methyl and ethyl for each repeating unit, wherein in at least one repeating unit R¹ is methyl, and in at least one repeating unit R¹ is ethyl.

In any of the above embodiments of formulas (II), (II′), (IIa), (IIa′), (IIb), and (IIb′), m preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 5 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments of formulas (II), (II′), (IIa), (IIa′), (IIb), and (IIb′), m is 2 to 180, such as 4 to 160, 6 to 140, 8 to 120 or 10 to 100, e.g., 20 to 80, 30 to 70, or 40 to 50.

In some embodiments of formulas (II), (II′), (IIa), (IIa′), (IIb), and (IIb′), L1 comprises at least one functional moiety, such as an alkylene moiety substituted with at least one monovalent functional moiety and/or linked, at the end by which the alkylene group is attached to R⁴, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide.

In some embodiments of formulas (II), (II′), (IIa), (IIa′), (IIb), and (IIb′), L1 comprises an alkylene moiety substituted with at least one monovalent functional moiety as specified above. Thus, in some embodiments, the conjugate may comprise the following structure (in particular, if the one or more hydrophobic chains are attached to the N-end (i.e., the terminal N atom) of the POX and/or POZ polymer, as shown, for example in formula (II)):
(hydrophobic chain)₁₋₂-(alkylene moiety substituted with at least one monovalent functional moiety)-(POX and/or POZ polymer), wherein “hydrophobic chain” represents R⁴; “alkylene moiety substituted with at least one monovalent functional moiety” represents L¹; and “POX and/or POZ polymer” represents the polymer specified in formula (I).

In some embodiments, the conjugate has the following formula (IIc) (in particular, if the one or more hydrophobic chains are attached to the N-end (i.e., the terminal N atom) of the POX and/or POZ polymer, as shown, for example in formula (II)):
(hydrophobic chain)₁₋₂-(alkylene moiety substituted with at least one monovalent functional moiety)-(POX and/or POZ polymer)-R³

In some embodiments, the at least one monovalent functional moiety may be any one of the monovalent functional moieties specified herein, e.g., selected from the groups consisting of hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide.

In some embodiments, the alkylene moiety substituted with at least one monovalent functional moiety is C₁₋₆-alkylene, such as C₁₋₃-alkylene, e.g., methylene, ethylene, or trimethylene.

In some embodiments, the alkylene moiety substituted with at least one monovalent functional moiety is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments, the alkylene moiety substituted with at least one monovalent functional moiety is C₁₋₆-alkylene, such as C₁₋₃-alkylene, e.g., methylene, ethylene, or trimethylene, and is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments of formulas (II), (II′), (IIa), (IIa′), (IIb), and (IIb′), L1 comprises an alkylene moiety linked, at the end by which the alkylene group is attached to R⁴, to a divalent functional moiety as specified above. Thus, in some embodiments, the conjugate may comprise the following structure (in particular, if the one or more hydrophobic chains are attached to the N-end (i.e., the terminal N atom) of the POX and/or POZ polymer, as shown, for example in formula (II)):
[(hydrophobic chain)-(divalent functional moiety)]₁₋₂-(alkylene moiety)-(POX and/or POZ polymer), wherein “hydrophobic chain” represents R⁴; “-(divalent functional moiety)]₁₋₂-(alkylene moiety)” represents L1; and “POX and/or POZ polymer” represents the polymer specified in formula (I).

In some embodiments, the conjugate has the following formula (IId) (in particular, if the one or more hydrophobic chains are attached to the N-end (i.e., the terminal N atom) of the POX and/or POZ polymer, as shown, for example in formula (II)):
[(hydrophobic chain)-(divalent functional moiety)]₁₋₂-(alkylene moiety)-(POX and/or POZ polymer)-R³

In some embodiments, the divalent functional moiety may be any one of the divalent functional moieties specified herein, e.g., selected from the groups consisting of ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide. In some embodiments, the linker comprises at least one divalent functional moiety selected from the group consisting of ester, sulfide, disulfide, sulfone, orthoester, acylhydrazone, hydrazine, oxime, acetal, ketal, amino, and amide moieties. In some preferred embodiments, the linker comprises at least one divalent functional moiety selected from the group consisting of ester, sulfide, sulfone, amino, and amide moieties.

In some embodiments, the alkylene moiety is C₁₋₆-alkylene, such as C₁₋₃-alkylene, e.g., methylene, ethylene, or trimethylene.

In some embodiments of formulas (II), (II′), (IIa), (IIa′), (IIb), and (IIb′), L1 comprises an alkylene moiety linked, at the end by which the alkylene group is attached to R⁴, to a trivalent functional moiety as specified above. Thus, in some embodiments, the conjugate may comprise the following structure (in particular, if the one or more hydrophobic chains are attached to the N-end (i.e., the terminal N atom) of the POX and/or POZ polymer, as shown, for example in formula (II)):
[(hydrophobic chain)₂-(trivalent functional moiety)]-(alkylene moiety)-(POX and/or POZ polymer), wherein “hydrophobic chain” represents R⁴; “-(trivalent functional moiety)]-(alkylene moiety)” represents L1; and “POX and/or POZ polymer” represents the polymer specified in formula (I).

In some embodiments, the conjugate has the following formula (IIe) (in particular, if the one or more hydrophobic chains are attached to the N-end (i.e., the terminal N atom) of the POX and/or POZ polymer, as shown, for example in formula (II)):
[(hydrophobic chain)₂-(trivalent functional moiety)]-(alkylene moiety)-(POX and/or POZ polymer)-R³

In some embodiments, the trivalent functional moiety may be any one of the trivalent functional moieties specified herein, e.g., selected from the groups consisting of amino, phosphate, sulfinamide, sulfonamide, sulfamate, sulfurous diamide, sulfuric diamide, urea, thiourea, orthoester, imidate, imino, imidothioate, thionylamido, guanidino (imidamido), carbamimidate, carbonimidate, carbamate, carbamodithioate, carbonodithioimidate, carbamimidothioate, carbamothioate, carbonimidothioate, acylhydrazone, hydrazine, acetal, ketal, hemiketal, imine, imide, and amide. In some embodiments, the linker comprises at least one trivalent functional moiety selected from the group consisting of phosphate, sulfonamide, sulfamate, amino, and amide moieties. In some preferred embodiments, the linker comprises at least one trivalent functional moiety selected from the group consisting of amino and amide moieties.

In some embodiments, the alkylene moiety is C₁₋₆-alkylene, such as C₁₋₃-alkylene, e.g., methylene, ethylene, or trimethylene.

In some embodiments of formulas (II), (II′), (IIa), (IIa′), (IIb), (IIb′), (IIc), and (IId) (in particular those, where the one or more hydrophobic chains are attached to the N-end (i.e., the terminal N atom) of the POX and/or POZ polymer, as shown, for example in formula (II)), L1 comprises at least one ester, sulfide, disulfide, sulfone, orthoester, acylhydrazone, hydrazine, oxime, acetal, ketal, or amide moiety. In certain embodiments, L¹ is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ—(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR²⁵)₃₋ᵣ](C₁₋₆-alkylene)-, [*—C(OR²⁵)₂O]ₚ(C₁₋₆-alkylene)-, [*—C(R²⁵)(═N—N(R²⁶)C(O)—)]ₚ(C₁₋₆-alkylene)-, [*—C(O)(N(R²⁶)—N═)C(R²⁵)—]ₚ(C₁₋₆-alkylene)-, [*═C(═N—N(R²⁶)C(O)(R²⁵))]ₚ(C₁₋₆-alkylene)-, [*—N(R²⁶) N(R²⁶)]ₚ(C₁₋₆-alkylene)-, [*═C(═N(OH))]ₚ(C₁₋₆-alkylene)-, and [*—OC(R²⁵)(R²⁶)O]ₚ(C₁₋₆-alkylene)-, wherein * represents the attachment point to R⁴; p is 1 or 2; C₁₋₆-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2. For example, L¹ may be selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, [*—SS]ₚ(C₁₋₃-alkylene)-, [*—S(O)₂]ₚ(C₁₋₃-alkylene)-, [(*—O)ᵣC(OR²⁵)₃₋ᵣ](C₁₋₃-alkylene)-, [*—C(OR²⁵)₂O]ₚ(C₁₋₃-alkylene)-, [*—C(R²⁵)(═N—N(R²⁶)C(O)—)]ₚ(C₁₋₃-alkylene)-, [*—C(O)(N(R²⁶)—N═)C(R²⁵)—]ₚ(C₁₋₃-alkylene)-, [*═C(═N—N(R²⁶)C(O)(R²⁵))]ₚ(C₁₋₃-alkylene)-, [*—N(R²⁶) N(R²⁶)]ₚ(C₁₋₃-alkylene)-, [*═C(═N(OH))]ₚ(C₁₋₃-alkylene)-, and [*—OC(R²⁵)(R²⁶)O]ₚ(C₁₋₃-alkylene)-, wherein * represents the attachment point to R⁴; p is 1 or 2; C₁₋₃-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2.

In some embodiments, R²⁵ is selected from the group consisting of C₁₋₃ alkyl, phenyl, and phenyl(C₁₋₃ alkyl), such as from the group consisting of methyl, ethyl, phenyl, benzyl, and phenylethyl.

In some embodiments, R²⁶ is selected from the group consisting of H, C₁₋₃ alkyl, phenyl, and phenyl(C₁₋₃ alkyl), such as from the group consisting of H, methyl, ethyl, phenyl, benzyl, and phenylethyl.

In some embodiments of formulas (II), (II′), (IIa), (IIa′), (IIb), (IIb′), (IIc), and (IId) (in particular those, where the one or more hydrophobic chains are attached to the N-end (i.e., the terminal N atom) of the POX and/or POZ polymer, as shown, for example in formula (II)), L¹ is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, preferably from the group consisting of [*—NHC(O)]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, more preferably from the group consisting of [*—NHC(O)]ₚ(C₁₋₆-alkylene)- and [*—C(O)O]ₚ(C₁₋₆-alkylene)-, wherein * represents the attachment point to R⁴; p is 1 or 2; and C₁₋₆-alkylene is either bivalent (if p is 1) or trivalent (if p is 2).

In some embodiments of formulas (II), (II′), (IIa), (IIa′), (IIb), (IIb′), (IIc), and (IId) (in particular those, where the one or more hydrophobic chains are attached to the N-end (i.e., the terminal N atom) of the POX and/or POZ polymer, as shown, for example in formula (II)), L¹ is selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ—(C₁₋₃-alkylene)-, [*—S]ₚ(C₁₋₃-alkylene)-, and [*—S(O)₂]ₚ(C₁₋₃-alkylene)-, preferably from the group consisting of [*—NHC(O)]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, and [*—S(O)₂]ₚ(C₁₋₃-alkylene)-, more preferably from the group consisting of [*—NHC(O)]ₚ(C₁₋₃-alkylene)- and [*—C(O)O]ₚ(C₁₋₃-alkylene)-, wherein * represents the attachment point to R⁴; p is 1 or 2; and C₁₋₃-alkylene is either bivalent (if p is 1) or trivalent (if p is 2).

In some embodiments of formulas (II), (II′), (IIa), (IIa′), (IIb), (IIb′), (IIc), and (IId) (in particular those, where the one or more hydrophobic chains are attached to the N-end (i.e., the terminal N atom) of the POX and/or POZ polymer, as shown, for example in formula (II)), L¹ is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)—(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R⁴. Thus, R² may be selected from the group consisting of R⁴, -L¹R⁴, —(CH₂)—CH(OC(O)R⁴)(CH₂OC(O)R⁴), —(CH₂)—CH(SR⁴)₂, and (CH₂)—CH(SR⁴)—CH₂(SR⁴); and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)—, preferably L1 is *—NHC(O)—(CH₂)— or *—NHC(O)—(CH₂)₂—, wherein * represents the attachment point to R⁴

In some embodiments of formulas (II), (II′), (IIa), (IIa′), (IIb), (IIb′), (IIc), (IId), and (IIe) (in particular those, where the one or more hydrophobic chains are attached to the N-end (i.e., the terminal N atom) of the POX and/or POZ polymer, as shown, for example in formula (II)), L1 is selected from the group consisting of [*-]₂NC(O)(C₁₋₆-alkylene)-, [*—C(O)O]ₚ(C₁₋₆-alkylene)-OP(O)₂O(C₁₋₆-alkylene) NHC(O)—(C₁₋₆-alkylene)-, [*—OC(O)]ₚ(C₁₋₆-alkylene)-OP(O)₂O(C₁₋₆-alkylene) NHC(O)—(C₁₋₆-alkylene)-, and [*—C(O)]ₚ(C₁₋₆-alkylene)-C(O)—, wherein * represents the attachment point to R⁴; p is 1 or 2; and C₁₋₆-alkylene is either bivalent (if p is 1) or trivalent (if p is 2). For example, L¹ may be selected from the group consisting of [*-]₂NC(O)(C₁₋₂-alkylene)-, [*—OC(O)]₂ (C₂₋₄-alkylene)-OP(O)₂O(C₂₋₃-alkylene) NHC(O)—(C₁₋₂-alkylene)-, and *—C(O)(C₂₋₃-alkylene)-C(O)—, wherein * represents the attachment point to R⁴.

In some embodiments of formulas (II), (IIa), and (IIb), and/or of formulas (IIc), (IId), and (IIe), R² is -L¹(R⁴)ₚ, i.e., the POX and/or POZ polymer is conjugated to the one or more hydrophobic chains (i.e., R⁴) via the linker L1.

In some embodiments of formulas (II′), (IIa′), and (IIb′), the linker comprises at least one difunctional moiety via which the one or more hydrophobic chains (R⁴) are attached to the C-end of the POX and/or POZ polymer. In some embodiments, the linker may additionally comprise an alkylene moiety (such as a C₁₋₆ alkylene moiety, e.g., a C₁₋₃ alkylene moiety), a cycloalkylene moiety (preferably a C₃₋₈-cycloalkylene, such as C₃₋₆-cycloalkylene moiety), or a cycloalkenylene moiety (preferably a C₃₋₈-cycloalkenylene, such as C₃₋₆-cycloalkenylene moiety) each of which connects the difunctional moiety to the C-end POX and/or POZ polymer (either directly to the C-end or, preferably, via a further difunctional moiety). For example, one hydrophobic chain (R⁴) may be attached to the C-end of the POX and/or POZ polymer via one difunctional moiety (either directly or via an alkylene, cycloalkylene, or cycloalkenylene moiety or via an alkylene, cycloalkylene, or cycloalkenylene moiety which bears another difunctional moiety); two hydrophobic chains (R⁴) may be attached to the C-end of the POX and/or POZ polymer via two difunctional moieties (which in turn are preferably attached to an alkylene, cycloalkylene, or cycloalkenylene moiety or to an alkylene, cycloalkylene, or cycloalkenylene moiety bearing another difunctional moiety); or two hydrophobic chains (R⁴) may be attached to the C-end of the POX and/or POZ polymer via the same difunctional moiety (which is then a trifunctional moiety and which may be attached to the C-end of the POX and/or POZ polymer either directly or via an alkylene, cycloalkylene, or cycloalkenylene moiety or to an alkylene, cycloalkylene, or cycloalkenylene moiety bearing another difunctional moiety). In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties. In some preferred embodiments of formulas (II′), (IIa′), and (IIb′), the linker comprises at least one divalent functional moiety selected from the group consisting of amide, sulfide, sulfone, and amino moieties.

In some embodiments of formulas (II′), (IIa′), and (IIb′), the cycloalkylene moiety is C₃₋₈-cycloalkylene, such as C₃₋₆-cycloalkylene, e.g., cyclopropylene, cyclobutylene, cyclopentylene, cyclohexylene, wherein the cycloalkylene moiety is optionally substituted (e.g., with one or more 1st level substituents, 2nd level substituents, or 3rd level substituents described herein), such as optionally substituted with one or more (e.g., 1, 2, 3, or 4) substituents selected from the group consisting of —OH, ═O, —SH, halogen, —CN, —N₃, and C₁₋₃-alkyl.

In some embodiments of formulas (II′), (IIa′), and (IIb′), the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein the cycloalkenylene moiety is optionally substituted (e.g., with one or more 1st level substituents, 2nd level substituents, or 3rd level substituents described herein), such as optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments of formulas (II′), (IIa′), and (IIb′), the alkylene moiety is C₁₋₆-alkylene, such as C₁₋₃-alkylene, e.g., methylene, ethylene, or trimethylene, or C₂₋₃ alkylene.

In some embodiments of formulas (II′), (IIa′), and (IIb′), the conjugate comprises one of the following structures:

(hydrophobic chain)-(divalent functional moiety)-(POX and/or POZ polymer) [(hydrophobic chain)-(divalent functional moiety)]₁₋₂-(alkylene moiety)-(divalent functional moiety)-(POX and/or POZ polymer)
(hydrophobic chain)-(divalent functional moiety)-(cycloalkylene moiety)-(divalent functional moiety)-(POX and/or POZ polymer)
(hydrophobic chain)-(divalent functional moiety)-(cycloalkenylene moiety)-(divalent functional moiety)-(POX and/or POZ polymer)
(hydrophobic chain)-(divalent functional moiety)-(alkylene moiety)-(POX and/or POZ polymer) [(hydrophobic chain)₂-(trivalent functional moiety)]-(alkylene moiety)-(divalent functional moiety)-(POX and/or POZ polymer)

In some embodiments of formulas (II′), (IIa′), and (IIb′), the conjugate has one of the following formulas (IIe′) to (IIj′):
(hydrophobic chain)-(divalent functional moiety)-(POX and/or POZ polymer)-R³ (IIe′) [(hydrophobic chain)-(divalent functional moiety)]₁₋₂-(alkylene moiety)-(divalent functional moiety)-(POX and/or POZ polymer)-R³ (IIf′)
(hydrophobic chain)-(divalent functional moiety)-(cycloalkylene moiety)-(divalent functional moiety)-(POX and/or POZ polymer)-R³ (IIg′)
(hydrophobic chain)-(divalent functional moiety)-(cycloalkenylene moiety)-(divalent functional moiety)-(POX and/or POZ polymer)-R³ (IIh′)
(hydrophobic chain)-(divalent functional moiety)-(alkylene moiety)-(POX and/or POZ polymer)-R³ (IIi′)
[(hydrophobic chain)₂-(trivalent functional moiety)]-(alkylene moiety)-(divalent functional moiety)-(POX and/or POZ polymer)-R³ (IIj′)

In some embodiments of formulas (II′), (IIa′), (IIb′), (IIe′), (IIf), (IIg′), (IIh′), (IIi′), and (IIj′), L¹ is selected from the group consisting of [*—Z]ₚ(C₁₋₆-alkylene)-Z—, *—Z—(C₃₋₈-cycloalkylene)-Z—, *—Z—(C₃₋₈-cycloalkenylene)-Z—, (*═N)(C₁₋₆-alkylene)-Z—, *—Z—(C₁₋₆-alkylene)-, and *—Z—, wherein * represents the attachment point to R⁴; p is 1 or 2; C₁₋₃-alkylene is either bivalent (if p is 1) or trivalent (if p is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(C₁₋₃-alkylene) NH—, —NH(C₁₋₃-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—, wherein R²² is selected from the group consisting of H, alkyl, alkenyl, alkynyl, cycloalkyl, aryl, heteroaryl, and heterocyclyl. For example, the linker can be selected from the group consisting of [*—C(O)O]ₚ(C₁₋₆-alkylene)-Z—, (*—NH) (C₁₋₆-alkylene)-Z—, (*═N)(C₁₋₆-alkylene)-Z—, (*—NH) C(O)(C₁₋₆-alkylene)-Z—, (*—C(O)NH(C₁₋₆-alkylene)-Z—, (*—NH) C(O)(C₁₋₆-alkylene)-, (*—C(O)NH(C₁₋₆-alkylene)-, (*—NH) C(O)—, *—C(O)NH—, *—Z—(C₃₋₈-cycloalkenylene)-Z—, —S—, and —S(O)₂—, wherein * represents the attachment point to R⁴; p is 1 or 2; C₁₋₆-alkylene is either bivalent (if p is 1) or trivalent (if p is 2); the C₃₋₈-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(C₁₋₃-alkylene) NH—, —NH(C₁₋₃-alkylene) OP(O)₂O—, —OC(O)NH—, —NHC(O)O—, —O—, —S—, and —NH—.

In some embodiments of formulas (II′), (IIa′), (IIb′), (IIe′), (IIf), (IIg′), (IIh′), (IIi′), and (IIj′), L1 is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment point to R⁴; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, the linker can be selected from the group consisting of [*—C(O)O]ₚ(C₁₋₃-alkylene)-Z—, (*—NH) (C₁₋₃-alkylene)-Z—, (*═N)(C₁₋₃-alkylene)-Z—, (*—NH) C(O)(C₁₋₃-alkylene)-Z—, (*—C(O)NH(C₁₋₃-alkylene)-Z—, (*—NH) C(O)(C₁₋₃-alkylene)-, (*—C(O)NH(C₁₋₃-alkylene)-, (*—NH) C(O)—, *—C(O)NH—, *—Z—(C₃₋₆-cycloalkenylene)-Z—, —S—, and —S(O)₂—, wherein * represents the attachment point to R⁴; p is 1 or 2; C₁₋₃-alkylene is either bivalent (if p is 1) or trivalent (if p is 2); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(C₁₋₂-alkylene) NH—, —NH(C₁₋₂-alkylene) OP(O)₂O—, —OC(O)NH—, —NHC(O)O—, —O—, —S—, and —NH—.

In some embodiments of formulas (II′), (IIa′), (IIb′), (IIe′), (IIf), (IIg′), (IIh′), (IIi′), and (IIj′), L¹ is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment to R⁴; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, the linker can be selected from the group consisting of [*—C(O)O]ₚ(C₁₋₃-alkylene)-Z—, (*—NH) (C₁₋₃-alkylene)-Z—, (*═N)(C₁₋₃-alkylene)-Z—, (*—NH) C(O)(C₁₋₃-alkylene)-Z—, (*—C(O)NH(C₁₋₃-alkylene)-Z—, (*—NH) C(O)(C₁₋₃-alkylene)-, (*—C(O)NH(C₁₋₃-alkylene)-, (*—NH) C(O)—, *—C(O)NH—, *—Z—(C₃₋₆-cycloalkenylene)-Z—, —S—, and —S(O)₂—, wherein * represents the attachment point to R⁴; p is 1 or 2; C₁₋₃-alkylene is either bivalent (if p is 1) or trivalent (if p is 2); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH—, —NHC(O)O—, —O—, —S—, and —NH—.

In some embodiments of formulas (II′), (IIa′), (IIb′), (IIe′), (IIff), (IIg′), (IIh′), (IIi′), and (IIj′), L¹ is selected from the group consisting of (*—C(O)O)(CH(OC(O)—*))(CH₂)—Z—, (*═N)(C₁₋₃-alkylene)-NHC(O)—, (*—Z) (C₁₋₃-alkylene)-Z—, *—Z—(C₃₋₆-cycloalkenylene)-Z—, and *—Z—, wherein * represents the attachment point to R⁴; the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, the linker can be selected from the group consisting of (*—C(O)O)(CH(OC(O)—*))(CH₂)—Z—, (*═N)(C₁₋₃-alkylene)-NHC(O)—, (*—NH) (C₁₋₃-alkylene)-NHC(O)—, *—C(O)NH—, (*—NH) C(O)—, *—Z—(C₃₋₆-cycloalkenylene)-Z—, —S—, and —S(O)₂—, wherein * represents the attachment point to R⁴; the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH—, —NHC(O)O—, —O—, —S—, and —NH.

In some embodiments of formulas (II′), (IIa′), (IIb′), (IIe′), (IIf), (IIg′), (IIh′), (Ili′), and (IIj′), L1 is selected from the group consisting of [*—C(O)]ₚ(C₁₋₆-alkylene)-C(O)NH— and [*-]₂N—, wherein * represents the attachment point to R⁴; p is 1 or 2; and C₁₋₆-alkylene is either bivalent (if p is 1) or trivalent (if p is 2). For example, the linker L¹ can be *—C(O)(C₂₋₃-alkylene)-C(O)NH— or [*-]₂N—, wherein * represents the attachment point to R⁴.

In some embodiments of formulas (II′), (IIa′), (IIb′), (IIe′), (IIf), (IIg′), (IIh′), (IIi′), and (IIj′), R² is selected from the group consisting of (R⁴C(O)O)(CH(OC(O)R⁴))(CH₂)—Z—, (R⁴)₂N(C₁₋₃-alkylene)-NHC(O)—, R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with one or more (e.g., 1, 2, 3, or 4) substituents independently selected from the group consisting of —OH. ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, the linker can be selected from the group consisting of (RC(O)O)(CH(OC(O)R⁴))(CH₂)—Z—, (R⁴)₂N(C₁₋₃-alkylene)-NHC(O)—, R⁴NH(C₁₋₃-alkylene)-NHC(O)—, R⁴C(O)NH—, (R⁴NH) C(O)—. R⁴Z—(C₃₋₆-cycloalkenylene)-Z—, R⁴S—, and R⁴S(O)₂—, wherein wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and Z is selected from the group consisting of —OP(O)₂O(CH₂)₂NH—, —NH(CH₂)₂OP(O)₂O—, —OC(O)NH—, —NHC(O)O—, —O—, —S—, —S(O)₂—, and —NH—.

In some embodiments of formulas (II′), (IIa′), (IIb′), (IIe′), (IIf), (IIg′), (IIh′), (IIi′), and (IIj′), R² is selected from the group consisting of R⁴—C(O)(CH₂)₂C(O)NH— and R⁴N—.

In some embodiments of formulas (II′), (IIa′), (IIb′), (IIe′), (IIf), (IIg′), (IIh′), (IIi′), and (IIj′), R² is -L¹(R⁴)ₚ, i.e., the POX and/or POZ polymer is conjugated to the one or more hydrophobic chains (i.e., R⁴) via the linker L1.

In any of the above embodiments of formulas (II), (II′), (IIa), (IIa′), (IIb), (IIb′), (IIc), (IId), (IIe), (IIe′), (IIf), (IIg′), (IIh′), (IIi′), and (IIj′), each R⁴ preferably is independently a non-cyclic, more preferably straight hydrocarbyl group. For example, each R⁴ is independently a hydrocarbyl group having at least 8 carbon atoms, such as at least 10 carbon atoms, preferably up to 30 carbon atoms, such as up to 28, 26, 24, 22, or 20 carbon atoms, or up to 16 carbon atoms, such as up to 15, 14, 13, 12, 11, or 10 carbon atoms. In some embodiments, each R⁴ is a hydrocarbyl group having 10 to 16 carbon atoms, such as 10 to 15 or 10 to 14 carbon atoms. In some embodiments, each R⁴ is a straight hydrocarbyl group having 10 to 16 carbon atoms, such as 10 to 15 or 10 to 14 carbon atoms.

Alternatively, in any of the above embodiments of formulas (II), (II′), (IIa), (IIa′), (IIb), (IIb′), (IIc), (IId), (IIe), (IIe′), (IIf), (IIg′), (IIh′), (IIi′), and (IIj′), one R⁴ may be a tocopherol moiety (which preferably means that in case more than one R⁴ is present in a respective R² substiuent, only one of the R⁴ substituents is a tocopherol moiety, whereas all remaining R⁴ substituents in the same R² substiuent are moieties other than a tocopherol moiety). For example, the one R⁴ may be an α-tocopheryl, β-tocopheryl, γ-tocopheryl, or 8-tocopheryl moiety. In some embodiments, the one R⁴ is an α-tocopheryl moiety.

In any of the above embodiments of formulas (II′), (IIa′), (IIb′), (IIe′), (IIff), (IIg′), (IIh′), (IIi′), and (IIj′), each R⁴ may preferably be a hydrocarbyl group having 10 to 18 carbon atoms, such as a straight alkyl group having 10 to 18 carbon atoms or a straight alkenyl group having 10 to 18 carbon atoms. For example, a straight alkyl group may have 10, 11, 12, 13, 14, 15, 16, 17, or 18 carbon atoms; and/or a straight alkenyl group may have 10, 11, 12, 13, 14, 15, 16, 17, or 18 carbon atoms and 1, 2, or 3 carbon-carbon double bonds.

In any of the above embodiments of formulas (II), (II′), (IIa), (IIa′), (IIb), (IIb′), (IIc), (IId), (IIe′), (IIf), (IIg′), (IIh′), (IIi′), and (IIj′) (and optionally (IIe)), R³ is preferably selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.

In any of the above embodiments of formulas (II), (II′), (IIa), (IIa′), (IIb), (IIb′), (IIc), (IId), (IIe′), (IIf), (IIg′), (IIh′), (IIi′), and (IIj′) (and optionally (IIe)), R³ is preferably selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³. —NR²²C(O)R²¹, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, —C(O)NR²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more (such as 1, 2, 3, or 4) substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group.

In any of the above embodiments of formulas (II), (II′), (IIa), (IIa′), (IIb), (IIb′), (IIc), (IId), (IIe′), (IIf), (IIg′), (IIh′), (IIi′), and (IIj′) (and optionally (IIe)), R³ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as 1 or 2) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In some embodiments of formulas (II), (IIa), (IIb), (IIc), and (IId), (and optionally (IIe)), R³ is selected from the group consisting of H, —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —C(O)NH₂, —C(O)NHCH₃, —OC(O)(CH₂)₂COOH, and a member of a targeting pair. In some embodiments of formulas (II), (IIa), (IIb), (IIc), and (IId), R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —C(O)NH₂, —C(O)NHCH₃, —OC(O)(CH₂)₂COOH, and a member of a targeting pair. In some embodiments of formulas (II), (IIa), (IIb), (IIc), and (IId), (and optionally (IIe)), R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair. In some embodiments of formulas (II), (IIa), (IIb), (IIc), and (IId) (and optionally (IIe)), R³ is selected from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.

In some embodiments of formulas (II′), (IIa′), (IIb′), (IIe′), (IIf), (IIg′), (IIh′), (IIi′), and (IIj′), R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair. In some embodiments of formulas (II′), (IIa′), (IIb′), (IIe′), (IIf), (IIg′), (IIh′), (IIi′), and (IIj′), R³ is C₁₋₃ alkyl optionally substituted with one substituent selected from the group consisting of —COOH, —COOCH₃, and —C(O)NH(CH₂)₂NH₂.

In certain embodiments, the conjugate has the following general formula (III) or (III′):

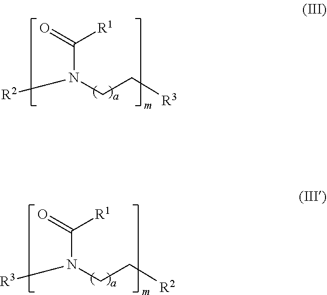

wherein:
a is an integer between 1 and 2;
R¹ is methyl or ethyl, and is independently selected for each repeating unit;
m is 10 to 100 (preferably 20 to 80, 30 to 70, or 40 to 50);
R², for formula (III), is selected from the group consisting of -L¹R+, —(CH₂)—CH(OC(O)R⁴)(CH₂OC(O)R⁴), —(CH₂)—CH(SR⁴)₂, and —(CH₂)—CH(SR⁴)—CH₂(SR⁴), wherein each R⁴ is independently a straight hydrocarbyl group having at least 10 carbon atoms (preferably having 10 to 16 carbon atoms); and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L¹ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴; or R², for formula (III′), is selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH. —COOCH₃, —NH₂, —NHCH₃, —N(CH₃)₂, —C(O)NH₂, —C(O)NHCH₃, —C(O)NH(CH₂)₂NH₂, and a member of a targeting pair.

In certain other embodiments, the conjugate has the general formula (III) or (III′), wherein:
a is an integer between 1 and 2;
R¹ is methyl or ethyl and is independently selected for each repeating unit;
m is 10 to 100;
R², for formula (III), is selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—, wherein each R⁴ is independently a straight hydrocarbyl group having at least 10 carbon atoms; or R², for formula (III′), is R⁴—C(O)(CH₂)₂C(O)NH— or R⁴N—, wherein each R⁴ is independently a straight hydrocarbyl group having at least 10 carbon atoms or a tocopherol moiety, wherein one R⁴ in the same R² moiety is a tocopherol moiety; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In some embodiments of formula (III) or (III′), a is 1, i.e., the conjugate has the following general formula (IIIa) or (IIIa′):

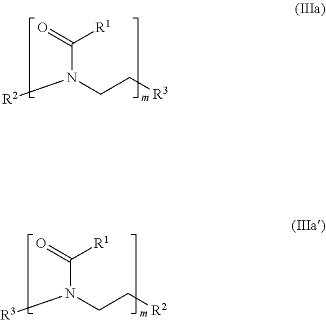

In some embodiments of formula (III) or (III′), a is 2, i.e., the conjugate has the following general formula (IIIb) or (IIIb′):

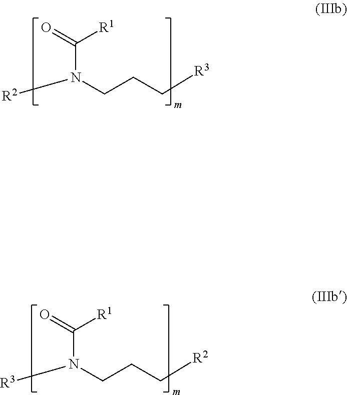

In any of the above embodiments of formulas (IIIa), (IIIa′), (IIIb), and (IIIb′), R¹, R², R³, and m are as defined for formula (III)/(III′).

In some embodiments of formulas (III), (III′), (IIIa), (IIIa′), (IIIb), and (IIIb′), each R¹ is methyl or each R¹ is ethyl. In some alternative embodiments of formulas (III), (III′), (IIIa), (IIIa′), (IIIb), and (IIIb′), R¹ is independently selected from methyl and ethyl for each repeating unit, wherein in at least one repeating unit R¹ is methyl, and in at least one repeating unit R¹ is ethyl.

In some embodiments of formulas (III), (IIIa), and (IIIb), R² is selected from the group consisting of R⁴. NHC(O)—(CH₂)—, R⁴—NHC(O)—(CH₂)₂—, —(CH₂)—CH(OC(O)R⁴)(CH₂OC(O)R⁴), —(CH₂)—CH(SR⁴)—CH₂(SR⁴), R⁴S—(CH₂)₃—, RAS(O)₂—(CH₂)₃—, and R⁴—OC(O)—(CH₂)—; and/or R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair (e.g., R³ is selected from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH).

In some embodiments of formulas (III), (IIIa), and (IIIb), R² is selected from the group consisting of R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—; and R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair (e.g., R³ is selected from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH).

In some embodiments of formulas (III′), (IIIa′), and (IIIb′), R² is selected from the group consisting of R⁴C(O)NH—, R⁴S—, R⁴S(O)₂—, and R⁴NH-(3,4-dioxocyclobut-1-en-1,2-diyl)-NH—; and/or R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair (e.g., R³ is C₁₋₃ alkyl optionally substituted with one substituent selected from the group consisting of —COOH, —COOCH₃, and —C(O)NH(CH₂)₂NH₂).

In some embodiments of formulas (III′), (IIIa′), and (IIIb′), R² is R⁴—C(O)(CH₂)₂C(O)NH— or R⁴N—; and R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair (e.g., R³ is C₁₋₃ alkyl optionally substituted with one substituent selected from the group consisting of —COOH, —COOCH₃, and —C(O)NH(CH₂)₂NH₂).

In some embodiments, the conjugate has the following general formula (IV):

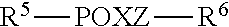

wherein:
R⁵ is R⁷ or -L¹(R⁷) q, wherein each R⁷ is independently a hydrocarbyl group; L² is a linker; and q is 1 or 2;
POXZ is a copolymer containing repeating units of the following general formulas (Ia) and (Ib):

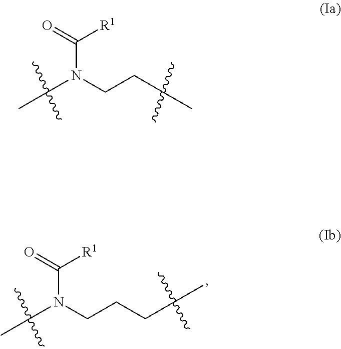

wherein each of R¹ is independently alkyl, in particular C₁₋₃ alkyl, such as methyl, ethyl, iso-propyl, or n-propyl, and is independently selected for each repeating unit; the number of repeating units of formula (Ia) in the copolymer is 1 to 199; the number of repeating units of formula (Ib) in the copolymer is 1 to 199; the sum of the number of repeating units of formula (Ia) and the number of repeating units of formula (Ib) in the copolymer is 2 to 200; and the repeating units of formulas (Ia) and (Ib) are arranged in a random, periodic, alternating or block wise manner; and
R⁶ is selected from the group consisting of H, C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂; a sugar, an amino acid, a peptide, and a member of a targeting pair.

In some embodiments, the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor. Thus, in some embodiments, a member of a targeting pair includes one of the following: an antigen, an antibody, avidin, streptavidin, folate, transferrin, an aptamer; an RGD peptide; an NGR peptide; and galactose.

In some of the above embodiments of formula (IV), R¹ at each occurrence (i.e., in each repeating unit) may be the same alkyl group (e.g., R¹ may be methyl in each repeating unit). In some alternative embodiments of formula (IV), R¹ in at least one repeating unit differs from R¹ in another repeating unit (e.g., for at least one repeating unit R¹ is one specific alkyl (such as ethyl), and for at least one different repeating unit R¹ is a different specific alkyl (such as methyl)). For example, each R¹ may be selected from two different alkyl groups (such as methyl and ethyl) and not all R¹ are the same alkyl.

In some embodiments of formula (IV), each of R¹ is independently methyl or ethyl, preferably methyl. Thus, in some embodiments of formula (IV), each R¹ is methyl or each R¹ is ethyl. In some alternative embodiments of formula (IV), R¹ is independently selected from methyl and ethyl for each repeating unit, wherein in at least one repeating unit R¹ is methyl, and in at least one repeating unit R¹ is ethyl.

In some embodiments of formula (IV), the sum of the number of repeating units of formula (Ia) and the number of repeating units of formula (Ib) in the copolymer preferably is between 2 and 190, such as between 2 and 180, between 2 and 170, between 2 and 160, between 2 and 150, between 2 and 140, between 2 and 130, between 2 and 120, between 2 and 110, between 2 and 100, between 2 and 90, between 2 and 80, between 2 and 70, between 4 and 200, between 4 and 190, between 4 and 180, between 4 and 170, between 4 and 160, between 4 and 150, between 4 and 140, between 4 and 130, between 4 and 120, between 4 and 110, between 4 and 100, between 4 and 90, between 4 and 80, between 4 and 70, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, or between 10 and 70. In certain embodiments of formula (IV), the sum of the number of repeating units of formula (Ia) and the number of repeating units of formula (Ib) in the copolymer is 2 to 180, such as 4 to 160, 6 to 140, 8 to 120 or 10 to 100, e.g., 20 to 80, 30 to 70, or 40 to 50.

Accordingly, in some embodiments of formula (IV), the number of repeating units of formula (Ia) in the copolymer is 1 to 179, such as 1 to 159, 1 to 139, 1 to 119 or 1 to 99; the number of repeating units of formula (Ib) in the copolymer is 1 to 179, such as 1 to 159, 1 to 139, 1 to 119 or 1 to 99; and the sum of the number of repeating units of formula (Ia) and the number of repeating units of formula (Ib) in the copolymer is 2 to 180, such as 4 to 160, 6 to 140, 8 to 120 or 10 to 100.

In some embodiments of formula (IV), L2 comprises at least one functional moiety, such as an alkylene moiety substituted with at least one monovalent functional moiety and/or linked, at the end by which the alkylene group is attached to R⁷, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide.

In some embodiments, R⁵ is attached to the N-end (i.e., the terminal N atom) of the POXZ copolymer and R⁶ is attached to the C-end (i.e., the terminal C atom) of the POXZ copolymer. In some alternative embodiments, R⁵ is attached to the C-end (i.e., the terminal C atom) of the POXZ copolymer and R⁶ is attached to the N-end (i.e., the terminal N atom) of the POXZ copolymer. The latter alternative embodiments of formula (IV) (i.e., where R⁵ is attached to the C-end (i.e., the terminal C atom) of the POXZ copolymer and R⁶ is attached to the N-end (i.e., the terminal N atom) of the POXZ copolymer) are designated as formula (IV′) herein.

In some embodiments of formula (IV), L2 comprises an alkylene moiety substituted with at least one monovalent functional moiety as specified above. Thus, in some embodiments, the conjugate may comprise the following structure (optionally R⁶ is attached to the terminal C atom of the POXZ copolymer):
(hydrophobic chain)₁₋₂-(alkylene moiety substituted with at least one monovalent functional moiety)-(POXZ copolymer), wherein “hydrophobic chain” represents R⁷; “alkylene moiety substituted with at least one monovalent functional moiety” represents L²; and “POXZ copolymer” represents the copolymer specified in formula (IV).

In some embodiments, the conjugate has the following formula (IVa) (optionally R⁶ is attached to the terminal C atom of the POXZ copolymer):
(hydrophobic chain)₁₋₂-(alkylene moiety substituted with at least one monovalent functional moiety)-(POXZ copolymer)-R⁶

In some embodiments, the at least one monovalent functional moiety may be any one of the monovalent functional moieties specified herein, e.g., selected from the groups consisting of hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, hydrazine, oxime, acetal, hemiacetal, ketal, hemiketal, imide, and amide.

In some embodiments, the alkylene moiety substituted with at least one monovalent functional moiety is C₁₋₆-alkylene, such as C₁₋₃-alkylene, e.g., methylene, ethylene, or trimethylene.

In some embodiments, the alkylene moiety substituted with at least one monovalent functional moiety is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments, the alkylene moiety substituted with at least one monovalent functional moiety is C₁₋₆-alkylene, such as C₁₋₃-alkylene, e.g., methylene, ethylene, or trimethylene, and is substituted with one or more (such as 1 to the maximum number of hydrogen atoms bound to the alkylene moiety, e.g., 1, 2, 3, 4, 5, or 6, such as between 1 to 5, 1 to 4, or 1 to 3, or 1 or 2) independently selected monovalent functional moieties.

In some embodiments of formula (IV) (in particular those, where R⁶ is attached to the terminal C atom of the POXZ copolymer), L² comprises an alkylene moiety linked, at the end by which the alkylene group is attached to R⁷, to a divalent functional moiety as specified above. Thus, in some embodiments (in particular those, where R⁶ is attached to the terminal C atom of the POXZ copolymer), the conjugate may comprise the following structure:
[(hydrophobic chain)-(divalent functional moiety)]₁₋₂-(alkylene moiety)-(POXZ copolymer), wherein “hydrophobic chain” represents R⁷; “-(divalent functional moiety)]₁₋₂-(alkylene moiety)” represents L²; and “POXZ copolymer” represents the copolymer specified in formula (IV).

In some embodiments (in particular those, where R⁶ is attached to the terminal C atom of the POXZ copolymer), the conjugate has the following formula (IVb):
[(hydrophobic chain)-(divalent functional moiety)]₁₋₂-(alkylene moiety)-(POXZ copolymer)-R⁶

In some embodiments, the divalent functional moiety may be any one of the divalent functional moieties specified herein, e.g., selected from the groups consisting of ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, hydrazine, oxime, acetal, hemiacetal, ketal, hemiketal, imine, imide, and amide.

In some embodiments, the alkylene moiety is C₁₋₆-alkylene, such as C₁₋₃-alkylene, e.g., methylene, ethylene, or trimethylene.

In some embodiments of formula (IV), L² comprises an alkylene moiety linked, at the end by which the alkylene group is attached to R⁷, to a trivalent functional moiety as specified above. Thus, in some embodiments (in particular those, where R⁶ is attached to the terminal C atom of the POXZ copolymer), the conjugate may comprise the following structure:
[(hydrophobic chain)₂-(trivalent functional moiety)]-(alkylene moiety)-(POXZ copolymer), wherein “hydrophobic chain” represents R⁷; “-(trivalent functional moiety)]-(alkylene moiety)” represents L²; and “POXZ copolymer” represents the copolymer specified in formula (IV).

In some embodiments (in particular those, where R⁶ is attached to the terminal C atom of the POXZ copolymer), the conjugate has the following formula (IVc):
[(hydrophobic chain)₂-(trivalent functional moiety)]-(alkylene moiety)-(POXZ copolymer)-R⁶

In some embodiments, the trivalent functional moiety may be any one of the trivalent functional moieties specified herein, e.g., selected from the groups consisting of amino, phosphate, sulfinamide, sulfonamide, sulfamate, sulfurous diamide, sulfuric diamide, urea, thiourea, orthoester, imidate, imino, imidothioate, thionylamido, guanidino (imidamido), carbamimidate, carbonimidate, carbamate, carbamodithioate, carbonodithioimidate, carbamimidothioate, carbamothioate, carbonimidothioate, acylhydrazone, hydrazine, acetal, ketal, hemiketal, imine, imide, and amide. In some embodiments, the linker comprises at least one trivalent functional moiety selected from the group consisting of phosphate, sulfonamide, sulfamate, amino, and amide moieties. In some preferred embodiments, the linker comprises at least one trivalent functional moiety selected from the group consisting of amino and amide moieties.

In some embodiments, the alkylene moiety is C₁₋₆-alkylene, such as C₁₋₃-alkylene, e.g., methylene, ethylene, or trimethylene.

In some embodiments of formula (IV), where R⁶ is attached to the terminal N atom of the POXZ copolymer (i.e., in the embodiments of formula (IV′)), L² comprises at least one difunctional moiety via which the one or more hydrophobic chains (R⁷) are attached to the POXZ copolymer. In some embodiments, L² may additionally comprise an alkylene moiety (such as a C₁₋₆ alkylene moiety, e.g., a C₁₋₃ alkylene moiety), a cycloalkylene moiety (preferably a C₃₋₈-cycloalkylene, such as C₃₋₆-cycloalkylene moiety), or a cycloalkenylene moiety (preferably a C₃₋₈-cycloalkenylene, such as C₃₋₆-cycloalkenylene moiety) each of which connects the difunctional moiety to the POXZ copolymer (either directly to the end of the POXZ copolymer or, preferably, via a further difunctional moiety). For example, one hydrophobic chain may be attached to the end of the POXZ copolymer via one difunctional moiety (either directly or via an alkylene, cycloalkylene, or cycloalkenylene moiety or via an alkylene, cycloalkylene, or cycloalkenylene moiety which bears another difunctional moiety); two hydrophobic chains may be attached to the end of the POXZ copolymer via two difunctional moieties (which in turn are preferably attached to an alkylene, cycloalkylene, or cycloalkenylene moiety or to an alkylene, cycloalkylene, or cycloalkenylene moiety bearing another difunctional moiety); or two hydrophobic chains may be attached to the end of the POXZ copolymer via the same difunctional moiety (which is then a trifunctional moiety and which may be attached to the end of the POXZ copolymer either directly or via an alkylene, cycloalkylene, or cycloalkenylene moiety or to an alkylene, cycloalkylene, or cycloalkenylene moiety bearing another difunctional moiety). In some embodiments, each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

In some embodiments, the cycloalkylene moiety is C₃₋₈-cycloalkylene, such as C₃₋₆-cycloalkylene, e.g., cyclopropylene, cyclobutylene, cyclopentylene, cyclohexylene, wherein the cycloalkylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments, the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein the cycloalkenylene moiety is optionally substituted with one or more (e.g., 1, 2, 3, or 4) substituents (e.g., independently selected from the group consisting of —OH, ═O, —SH, halogen, —CN, —N₃, and C₁₋₃-alkyl).

In some embodiments, the alkylene moiety is C₁₋₆-alkylene, such as C₁₋₃-alkylene, e.g., methylene, ethylene, or trimethylene, or C₂₋₃ alkylene.

In some embodiments of formula (IV′), the conjugate comprises one of the following structures:
(hydrophobic chain)-(divalent functional moiety)-(POX and/or POZ polymer)
[(hydrophobic chain)-(divalent functional moiety)]₁₋₂-(alkylene moiety)-(divalent functional moiety)-(POX and/or POZ polymer)
(hydrophobic chain)-(divalent functional moiety)-(cycloalkylene moiety)-(divalent functional moiety)-(POX and/or POZ polymer)
(hydrophobic chain)-(divalent functional moiety)-(cycloalkenylene moiety)-(divalent functional moiety)-(POX and/or POZ polymer)
(hydrophobic chain)-(divalent functional moiety)-(alkylene moiety)-(POX and/or POZ polymer)
[(hydrophobic chain)₂-(trivalent functional moiety)]-(alkylene moiety)-(divalent functional moiety)-(POX and/or POZ polymer)

In some embodiments formula (IV′), the conjugate has one of the following formulas (IVc′) to (IVh′):
(hydrophobic chain)-(divalent functional moiety)-(POXZ copolymer)-R⁶ (IVc′)
[(hydrophobic chain)-(divalent functional moiety) ]₁₋₂-(alkylene moiety)-(divalent functional moiety)-(POXZ copolymer)-R⁶ (IVd′)
(hydrophobic chain)-(divalent functional moiety)-(cycloalkylene moiety)-(divalent functional moiety)-(POXZ copolymer)-R⁶ (IVe′)
(hydrophobic chain)-(divalent functional moiety)-(cycloalkenylene moiety)-(divalent functional moiety)-(POXZ copolymer)-R⁶ (IVf)
(hydrophobic chain)-(divalent functional moiety)-(alkylene moiety)-(POXZ copolymer)-R⁶ (IVg′)
[(hydrophobic chain)₂-(trivalent functional moiety)]-(alkylene moiety)-(divalent functional moiety)-(POXZ copolymer)-R⁶ (IVh′)

In some embodiments of formulas (IV), (IVa), and (IVb), L² comprises at least one ester, sulfide, disulfide, sulfone, orthoester, acylhydrazone, hydrazine, oxime, acetal, ketal, or amide moiety. In some embodiments, L² is selected from the group consisting of [*—NHC(O)]_{q}(C₁₋₆-alkylene)-, [*—C(O)NH]_{q}(C₁₋₆-alkylene)-, [*—C(O)O], (C₁₋₆-alkylene)-, [*—OC(O)], (C₁₋₆-alkylene)-, [*—S]_{q}(C₁₋₆-alkylene)-, [*—SS]_{q}(C₁₋₆-alkylene)-, [*—S(O)₂]ₚ(C₁₋₆-alkylene)-, [(*—O)ₛC(OR²⁵)₃₋ₛ](C₁₋₆-alkylene)-, [*—C(OR²⁵)₂O]_{q}(C₁₋₆-alkylene)-, [*—C(R²⁵)(═N—N(R²⁶)C(O)—)], (C₁₋₃-alkylene)-, [*—C(O)(N(R²⁶)—N═)C(R²⁵)—]_{q}(C₁₋₃-alkylene)-, [*═C(═N—N(R²⁶)C(O)(R²⁵))]_{q}(C₁₋₃-alkylene)-, [*—N(R²⁶) N(R²⁶)]_{q}—(C₁₋₆-alkylene)-, [*═C(═N(OH))], (C₁₋₆-alkylene)-, and [*—OC(R²⁵)(R²⁶)O], (C₁₋₆-alkylene)-, wherein * represents the attachment point to R⁷; q is 1 or 2; C₁₋₆-alkylene is either bivalent (if q is 1) or trivalent (if q is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and s is an integer between 1 and 2. For example, L² may be selected from the group consisting of [*—NHC(O)], (C₁₋₃-alkylene)-, [*—C(O)NH], (C₁₋₃-alkylene)-, [*—C(O)O], (C₁₋₃-alkylene)-, [*—OC(O)], (C₁₋₃-alkylene)-, [*—S], (C₁₋₃-alkylene)-, [*—SS], (C₁₋₃-alkylene)-, [*—S(O)₂]ₚ(C₁₋₃-alkylene)-, [(*—O)ₛC(OR²⁵)₃₋ₛ](C₁₋₃-alkylene)-, [*—C(OR²⁵)₂O]_{q}(C₁₋₃-alkylene)-, [*—C(R²⁵)(═N—N(R²⁶)C(O)—)]_{q}(C₁₋₃-alkylene)-, [*—C(O)(N(R²⁶)—N═)C(R²⁵)—]_{q}(C₁₋₃-alkylene)-, [*═C(═N—N(R²⁶)C(O)(R²⁵))], (C₁₋₃-alkylene)-, [*—N(R²⁶) N(R²⁶)], (C₁₋₃-alkylene)-, [*═C(═N(OH))], (C₁₋₃-alkylene)-, and [*—OC(R²⁵)(R²⁶)O]_{q}(C₁₋₃-alkylene)-, wherein * represents the attachment point to R⁷; q is 1 or 2; C₁₋₃-alkylene is either bivalent (if q is 1) or trivalent (if q is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and s is an integer between 1 and 2.

In some embodiments, R²⁵ is selected from the group consisting of C₁₋₃ alkyl, phenyl, and phenyl(C₁₋₃ alkyl), such as from the group consisting of methyl, ethyl, phenyl, benzyl, and phenylethyl.

In some embodiments, R²⁶ is selected from the group consisting of H, C₁₋₃ alkyl, phenyl, and phenyl(C₁₋₃ alkyl), such as from the group consisting of H, methyl, ethyl, phenyl, benzyl, and phenylethyl.

In some embodiments of formulas (IV), (IVa), and (IVb), L² is selected from the group consisting of [*—NHC(O)]_{q}(C₁₋₆-alkylene)-, [*—C(O)NH], (C₁₋₆-alkylene)-, [*—C(O)O]_{q}(C₁₋₆-alkylene)-, [*—OC(O)], (C₁₋₆-alkylene)-, [*—S], (C₁₋₆-alkylene)-, and [*—S(O)₂]ₚ(C₁₋₆-alkylene)-, preferably from the group consisting of [*—NHC(O)], C₁₋₆-alkylene)-, [*—C(O)O], (C₁₋₆-alkylene)-, [*—OC(O)], (C₁₋₆-alkylene)-, [*—S]_{q}(C₁₋₆-alkylene)-, and [*—S(O)₂]ₚ(C₁₋₆-alkylene)-, more preferably from the group consisting of [*—NHC(O)]_{q}(C₁₋₆-alkylene)- and [*—C(O)O], (C₁₋₆-alkylene)-, wherein * represents the attachment point to R⁷; q is 1 or 2; and C₁₋₆-alkylene is either bivalent (if q is 1) or trivalent (if q is 2).

In some embodiments of formulas (IV), (IVa), and (IVb), L² is selected from the group consisting of [*—NHC(O)], (C₁₋₃-alkylene)-, [*—C(O)NH], (C₁₋₃-alkylene)-, [*—C(O)O]_{q}(C₁₋₃-alkylene)-, [*—OC(O)], (C₁₋₃-alkylene)-, [*—S], (C₁₋₃-alkylene)-, and [*—S(O)₂]ₚ(C₁₋₃-alkylene)-, preferably from the group consisting of [*—NHC(O)]_{q}(C₁₋₃-alkylene)-, [*—C(O)O], C₁₋₃-alkylene)-, [*—OC(O)], (C₁₋₃-alkylene)-, [*—S], (C₁₋₃-alkylene)-, and [*—S(O)₂]ₚ(C₁₋₃-alkylene)-, more preferably from the group consisting of [*—NHC(O)], (C₁₋₃-alkylene)- and [*—C(O)O], (C₁₋₃-alkylene)-, wherein * represents the attachment point to R⁷; q is 1 or 2; and C₁₋₃-alkylene is either bivalent (if q is 1) or trivalent (if q is 2).

In some embodiments of formulas (IV), (IVa), and (IVb), L² is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R⁷. Thus, R⁵ may be selected from the group consisting of R⁷, -L²R⁷, —(CH₂)—CH(OC(O)R⁷)(CH₂OC(O)R⁷), —(CH₂)—CH(SR⁷)₂, and —(CH₂)—CH(SR⁷)—CH₂(SR⁷); and L² is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)—, preferably L² is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—, wherein * represents the attachment point to R⁷.

In some embodiments of formulas (IV), (IVa), (IVb), and (IVc), L² is selected from the group consisting of [*-]₂NC(O)(C₁₋₆-alkylene)-, [*—C(O)O], (C₁₋₆-alkylene)-OP(O)₂O(C₁₋₆-alkylene) NHC(O)—(C₁₋₆-alkylene)-, [*—OC(O)], (C₁₋₆-alkylene)-OP(O)₂O(C₁₋₆-alkylene) NHC(O)—(C₁₋₆-alkylene)-, and [*—C(O)]_{q}(C₁₋₆-alkylene)-C(O)—, wherein * represents the attachment point to R⁷; q is 1 or 2; and C₁₋₆-alkylene is either bivalent (if q is 1) or trivalent (if q is 2). For example, L² may be selected from the group consisting of [*-]₂NC(O)(C₁₋₂-alkylene)-, [*—OC(O)]₂ (C₂₋₄-alkylene)-OP(O)₂O(C₂₋₃-alkylene) NHC(O)—(C₁₋₂-alkylene)-, and *—C(O)(C₂₋₃-alkylene)-C(O)—, wherein * represents the attachment point to R⁷.

In some embodiments of formulas (IV), (IVa), and (IVb) (and optionally (IVc)), R⁵ is-L² (R⁵) q, i.e., the POXZ copolymer is conjugated to the one or more hydrophobic chains (i.e., R⁷) via the linker L².

In some embodiments of formulas (IV′), (IVc′), (IVd′), (IVe′), (IVf), (IVg′) and (IVh′), L² is selected from the group consisting of [*—Z¹], (C₁₋₆-alkylene)-Z¹—, *—Z¹—(C₃₋₈-cycloalkylene)-Z¹—, *—Z¹—(C₃₋₈-cycloalkenylene)-Z¹—, (*═N)(C₁₋₆-alkylene)-Z¹—, *—Z¹ (C₁₋₆-alkylene)-, and *—Z¹—, wherein * represents the attachment point to R⁷; q is 1 or 2; C₁₋₆-alkylene is either bivalent (if q is 1) or trivalent (if q is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(C₁₋₃-alkylene) NH—, —NH(C₁₋₃-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²². For example, L² can be selected from the group consisting of [*—C(O)O], (C₁₋₆-alkylene)-Z¹—, (*—NH) (C₁₋₆-alkylene)-Z¹—, (*═N)(C₁₋₆-alkylene)-Z¹—, (*—NH) C(O)(C₁₋₆-alkylene)-Z¹—, (*—C(O)NH(C₁₋₆-alkylene)-Z¹—, (*—NH) C(O)(C₁₋₆-alkylene)-, (*—C(O)NH(C₁₋₆-alkylene)-, *—Z¹—(C₃₋₈-cycloalkenylene)-Z¹—, *—S—, *—S(O)₂—, (*—NH) C(O)—, and *—C(O)NH—, wherein * represents the attachment point to R⁷; q is 1 or 2; C₁₋₆-alkylene is either bivalent (if q is 1) or trivalent (if q is 2); and Z¹ is selected from the group consisting of —OP(O)₂O(C₁₋₃-alkylene) NH—, —NH(C₁₋₃-alkylene) OP(O)₂O—, —OC(O)NH—, —NHC(O)O—, —O—, —S—, and —NH—.

In some embodiments of formulas (IV′), (IVc′), (IVd′), (IVe′), (IVf), (IVg′) and (IVh′), L² is selected from the group consisting of [*—Z¹], (C₁₋₃-alkylene)-Z¹—, *—Z¹—(C₃₋₆-cycloalkylene)-Z¹—, *—Z¹—(C₃₋₆-cycloalkenylene)-Z¹—, (*═N)(C₁₋₃-alkylene)-Z¹—, *—Z¹ (C₁₋₃-alkylene)-, and *—Z¹—, wherein * represents the attachment point to R⁷; q is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(C₁₋₂-alkylene) NH—, —NH(C₁₋₂-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²². For example, L² can be selected from the group consisting of [*—C(O)O]_{q}(C₁₋₃-alkylene)-Z¹—, (*—NH) (C₁₋₃-alkylene)-Z¹—, (*═N)(C₁₋₃-alkylene)-Z¹—, (*—NH) C(O)(C₁₋₃-alkylene)-Z¹—, (*—C(O)NH(C₁₋₃-alkylene)-Z¹—, (*—NH) C(O)(C₁₋₃-alkylene)-, (*—C(O)NH(C₁₋₃-alkylene)-, *—Z¹—(C₃₋₆-cycloalkenylene)-Z¹—, *—S—, *—S(O)₂—, (*—NH) C(O)—, and *—C(O)NH—, wherein * represents the attachment point to R⁷; q is 1 or 2; C₁₋₃-alkylene is either bivalent (if q is 1) or trivalent (if q is 2); and Z¹ is selected from the group consisting of —OP(O)₂O(C₁₋₂-alkylene) NH—, —NH(C₁₋₂-alkylene) OP(O)₂O—, —OC(O)NH—, —NHC(O)O—, —O—, —S—, and —NH—.

In some embodiments of formulas (IV′), (IVc′), (IVd′), (IVe′), (IVf), (IVg′) and (IVh′), L² is selected from the group consisting of [*—Z¹], (C₁₋₃-alkylene)-Z¹—, *—Z¹—(C₃₋₆-cycloalkylene)-Z¹—, *—Z¹—(C₃₋₆-cycloalkenylene)-Z¹—, (*═N)(C₁₋₃-alkylene)-Z¹—, *—Z¹ (C₁₋₃-alkylene)-, and *—Z¹—, wherein * represents the attachment point to R⁷; p is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O): O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—. For example, L² can be selected from the group consisting of [*—C(O)O]_{q}(C₁₋₃-alkylene)-Z¹—, (*—NH) (C₁₋₃-alkylene)-Z¹—, (*═N)(C₁₋₃-alkylene)-Z¹—, (*—NH) C(O)(C₁₋₃-alkylene)-Z¹—, (*—C(O)NH(C₁₋₃-alkylene)-Z¹—, (*—NH) C(O)(C₁₋₃-alkylene)-, (*—C(O)NH(C₁₋₃-alkylene)-, *—Z¹—(C₃₋₆-cycloalkenylene)-Z¹—, *—S—, *—S(O)₂—, (*—NH) C(O)—, and *—C(O)NH—, wherein * represents the attachment point to R⁷; p is 1 or 2; C₁₋₃-alkylene is either bivalent (if q is 1) or trivalent (if q is 2); and Z¹ is selected from the group consisting of —OP(O)₂O(CH₂)₂NH—, —NH(CH₂)₂OP(O)₂O—, —OC(O)NH—, —NHC(O)O—, —O—, —S—, and —NH—.

In some embodiments of formulas (IV′), (IVc′), (IVd′), (IVe′), (IVf), (IVg′) and (IVh′), L² is selected from the group consisting of (*—C(O)O)(CH(OC(O)—*))(CH₂)—Z¹—, (*═N)(C₁₋₃-alkylene)-NHC(O)—, (*—Z¹ (C₁₋₃-alkylene)-Z¹—, *—Z¹—(C₃₋₆-cycloalkenylene)-Z¹—, and *—Z¹—, wherein * represents the attachment point to R⁷; the C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂—NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. For example, L² can be selected from the group consisting of (*—C(O)O)(CH(OC(O)—*))(CH₂)—Z¹—, (*═N)(C₁₋₃-alkylene)-NHC(O)—, (*—NH) (C₁₋₃-alkylene)-NHC(O)—, *—Z¹—(C₃₋₆-cycloalkenylene)-Z¹—, *—S—, *—S(O)₂—, and *—C(O)NH—, wherein * represents the attachment point to R⁷; and Z¹ is selected from the group consisting of —OP(O)₂O(CH₂)₂NH—, —NH(CH₂)₂OP(O)₂—O—, —OC(O)NH—, —NHC(O)O—, —O—, —S—, and —NH—. Thus, in some embodiments of formulas (IV′), (IVc′), (IVd′), (IVe′), (IVf), (IVg′) and (IVh′), R⁵ is selected from the group consisting of (R⁴C(O)O)(CH(OC(O)R⁷))(CH₂)—Z¹—, (R⁷)₂N(C₁₋₃-alkylene)-Z¹—, R⁷Z¹ (C₁₋₃-alkylene)-Z¹—, R⁷Z¹—(C₃₋₆-cycloalkenylene)-Z¹—, and R⁷Z¹—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—. —NHC(O)O—, —O—, —C(O)O—, —OC(O)—, —S—, —S(O)₂—, and —NH—. For example, R⁵ can be selected from the group consisting of (R⁷C(O)O)(CH(OC(O)R⁷))(CH₂)—Z¹—, (R⁷)₂N(C₁₋₃-alkylene)-NHC(O)—, R⁷NH(C₁₋₃-alkylene) NHC(O)—, R⁷Z¹—(C₃₋₆-cycloalkenylene)-Z¹—, R⁷S—, R⁷S(O)₂—, and RIC(O)NH—, wherein Z¹ is selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.

In some embodiments of formulas (IV′), (IVc′), (IVd′), (IVe′), (IVf), (IVg′) and (IVh′), L² is selected from the group consisting of [*—C(O)], (C₁₋₆-alkylene)-C(O)NH— and [*-]₂N—, wherein * represents the attachment point to R⁷; q is 1 or 2; and C₁₋₆-alkylene is either bivalent (if q is 1) or trivalent (if q is 2). For example, L² can be *—C(O)(C₂₋₃-alkylene)-C(O)NH— or [*-]₂N—, wherein * represents the attachment point to R⁷. Thus, in some embodiments of formulas (IV′), (IVc′), (IVd′), (IVe′), (IVf), (IVg′) and (IVh′), R⁵ is selected from the group consisting of R⁷—C(O)(CH₂)₂C(O)NH— and R⁷N—.

In some embodiments of formulas (IV′), (IVc′), (IVd′), (IVe′), (IVf), (IVg′) and (IVh′), R⁵ is-L¹(R⁵)_{q}. i.e., the POXZ copolymer is conjugated to the one or more hydrophobic chains (i.e., R⁷) via the linker L².

In any of the above embodiments of formulas (IV), (IVa), (IVb), (IV′), (IVc′), (IVd′), (IVe′), (IVf), (IVg′) and (IVh′) (and optionally (IVc)), R⁷ preferably is independently a non-cyclic, preferably straight hydrocarbyl group. For example, each R⁷ preferably is independently a hydrocarbyl group having at least 8 carbon atoms, such as at least 10 carbon atoms, preferably up to 30 carbon atoms, such as up to 28, 26, 24, 22, or 20 carbon atoms, or up to 16 carbon atoms, such as up to 15, 14, 13, 12, 11, or 10 carbon atoms. In some embodiments, each R⁷ is a hydrocarbyl group having 10 to 16 carbon atoms, such as 10 to 15 or 10 to 14 carbon atoms. In some embodiments, each R⁷ is a straight hydrocarbyl group having 10 to 16 carbon atoms, such as 10 to 15 or 10 to 14 carbon atoms.

Alternatively, in any of the above embodiments of formulas (IV), (IVa), (IVb), (IVc), (IV′), (IVc′), (IVd′), (IVe′), (IVf), (IVg′) and (IVh′), one R⁷ may be a tocopherol moiety (which preferably means that in case more than one R⁷ is present in a respective R⁵ substiuent, only one of the R⁷ substituents is a tocopherol moiety, whereas all remaining R⁷ substituents in the same R⁵ substiuent are moieties other than a tocopherol moiety). For example, the one R⁷ may be an α-tocopheryl, β-tocopheryl, γ-tocopheryl, or 8-tocopheryl moiety. In some embodiments, the one R⁷ is an α-tocopheryl moiety.

In any of the above embodiments of formulas (IV′), (IVc′), (IVd′), (IVe′), (IVf), (IVg′) and (IVh′), each R⁷ may preferably be a hydrocarbyl group having 10 to 18 carbon atoms, such as a straight alkyl group having 10 to 18 carbon atoms or a straight alkenyl group having 10 to 18 carbon atoms. For example, straight alkenyl group may have 10, 11, 12, 13, 14, 15, 16, 17, or 18 carbon atoms and 1, 2, or 3 carbon-carbon double bonds.

In any of the above embodiments of formulas (IV), (IVa), (IVb), (IV′), (IVc′), (IVd′), (IVe′), (IVf), (IVg′) and (IVh′) (and optionally (IVc)), R⁶ is preferably selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, and a member of a targeting pair;
R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.

In any of the above embodiments of formulas (IV), (IVa), (IVb), (IV′), (IVc′), (IVd′), (IVe′), (IVf), (IVg′) and (IVh′) (and optionally (IVc)), R⁶ is preferably selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, —C(O)NR²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group.

In any of the above embodiments of formulas (IV), (IVa), (IVb), (IV′), (IVc′), (IVd′), (IVe′), (IVf), (IVg′) and (IVh′) (and optionally (IVc)), R⁶ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃). —NHC(O)(CH₂)₂COOH, —N(CH₂CH₃)C(O)(CH₂)₂COOH, —N(CH₂CH₃)C(O) CH₃, —C(O)NH₂. —C(O)NHCH₃, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In some embodiments of formulas (IV), (IVa), and (IVb), (and optionally (IVc)), R⁶ is selected from the group consisting of H, —OH, —N₃, C₂₋₆ alkynyl, —COOH, —NH₂, —NHCH₃, —N(CH₃)₂, —NHC(O)(CH₂)₂COOH, Z—N(CH₂CH₃)C(O)(CH₂)₂COOH, —N(CH₂CH₃)C(O) CH₃, —NH(CH₂CH₃), —C(O)NH₂, —C(O)NHCH₃, —OC(O)(CH₂)₂COOH, and a member of a targeting pair. In some embodiments of formulas (IV), (IVa), and (IVb), R⁶ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —C(O)NH₂, —C(O)NHCH₃, —OC(O)(CH₂)₂COOH, and a member of a targeting pair. In some embodiments of formulas (IV), (IVa), and (IVb) (and optionally (IVc)), R⁶ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair. In some embodiments of formulas (IV), (IVa), and (IVb), R⁶ is selected from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.

In some embodiments of formulas (IV′), (IVc′), (IVd′), (IVe′), (IVf), (IVg′) and (IVh′), R⁶ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, Z—C(O)NH(CH₂)₂NH₂, and a member of a targeting pair. In some embodiments of formulas (IV′), (IVc′), (IVd′), (IVe′), (IVf), (IVg′) and (IVh′), R⁶ is C₁₋₃ alkyl optionally substituted with one substituent selected from the group consisting of —COOH, —COOCH₃, and —C(O)NH(CH₂)₂NH₂.

In certain embodiments, the conjugate has the following general formula (V):

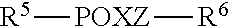

wherein:
R⁵, when attached to the N-end of the POXZ copolymer, is selected from the group consisting of -L²R⁷, —(CH₂)—CH(OC(O)R⁷)(CH₂OC(O)R⁷), —(CH₂)—CH(SR⁷)₂, and —(CH₂)—CH(SR⁷)—CH₂(SR⁷), wherein each R⁷ is independently a straight hydrocarbyl group having at least 10 carbon atoms (preferably having 10 to 15 carbon atoms); and L² is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L² is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁷, or R⁵, when attached to the C-end of the POXZ copolymer, is selected from the group consisting of (R⁷C(O)O)(CH(OC(O)R⁷))(CH₂)—Z¹—, (R⁷)₂N(C₁₋₃-alkylene)-Z¹—, R⁷Z¹ (C₁₋₃-alkylene)-Z¹—, R⁷Z¹—(C₃₋₆-cycloalkenylene)-Z¹—, and R⁷Z¹—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; POXZ is a copolymer containing the repeating units of the following general formulas (Ia) and (Ib):

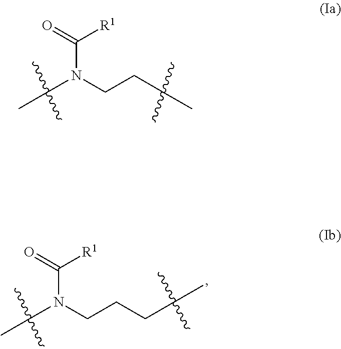

wherein each of R¹ is independently methyl or ethyl, and is independently selected for each repeating unit; the number of repeating units of formula (Ia) in the copolymer is 1 to 99 (preferably 1 to 79, 1 to 69, or 1 to 49); the number of repeating units of formula (Ib) in the copolymer is 1 to 99 (preferably 1 to 79, 1 to 69, or 1 to 49); the sum of the number of repeating units of formula (Ia) and the number of repeating units of formula (Ib) in the copolymer is 10 to 100 (preferably 20 to 80, 30 to 70, or 40 to 50); and the repeating units of formulas (Ia) and (Ib) are arranged in a random, periodic, alternating or block wise manner; and
R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In certain embodiments, the conjugate has the general formula (V), wherein:
R⁵, when attached to the N-end of the POXZ copolymer, is selected from the group consisting of (R⁷)₂NC(O)(C₁₋₂-alkylene)-, R⁷C(O)OCH₂CH(OC(O)R⁷)—CH₂—OP(O)₂O—(CH₂)₂—NHC(O)—CH₂—, and R⁷—C(O)(CH₂)₂—C(O)—, wherein each R⁷ is independently a straight hydrocarbyl group having at least 10 carbon atoms, or R⁵, when attached to the C-end of the POXZ copolymer, is R⁷—C(O)(CH₂)₂C(O)NH— or R⁷N—, wherein each R⁷ is independently a straight hydrocarbyl group having at least 10 carbon atoms or a tocopherol moiety, wherein one R⁷ in the same R⁵ moiety is a tocopherol moiety; POXZ is a copolymer containing the repeating units of the general formulas (Ia) and (Ib), wherein each of R¹ is independently methyl or ethyl and is independently selected for each repeating unit; the number of repeating units of formula (Ia) in the copolymer is 1 to 99; the number of repeating units of formula (Ib) in the copolymer is 1 to 99; the sum of the number of repeating units of formula (Ia) and the number of repeating units of formula (Ib) in the copolymer is 10 to 100; and the repeating units of formulas (Ia) and (Ib) are arranged in a random, periodic, alternating or block wise manner; and
R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

Those embodiments of formula (V), where R⁵ is attached to the C-end (i.e., the terminal C atom) of the POXZ copolymer and R⁶ is attached to the N-end (i.e., the terminal N atom) of the POXZ copolymer), are designated as formula (V′) herein.

In some embodiments of formula (V) or (V′), each R¹ is methyl or each R¹ is ethyl. In some alternative embodiments of formula (V) or (V′), R¹ is independently selected from methyl and ethyl for each repeating unit, wherein in at least one repeating unit R¹ is methyl, and in at least one repeating unit R¹ is ethyl.

In some embodiments of formula (V), where R⁵ is attached to the N-end of the POXZ copolymer, R⁵ is selected from the group consisting of R⁷—NHC(O)—(CH₂)—, R⁷—NHC(O)—(CH₂)₂—, —(CH₂)—CH(OC(O)R⁷)(CH₂OC(O)R⁷), —(CH₂)—CH(SR⁷)—CH₂(SR⁷), R⁷S—(CH₂)₃—, R⁷S(O)₂—(CH₂)₃—, and R⁷—OC(O)—(CH₂)—; and/or R⁶ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair (e.g., R⁶ is selected from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH).

In some embodiments of formula (V), where R⁵ is attached to the N-end of the POXZ copolymer, R⁵ is selected from the group consisting of (R⁷)₂NC(O)(C₁₋₂-alkylene)-, R⁷C(O)OCH₂CH(OC(O)R⁴)—CH₂—OP(O)₂O—(CH₂)₂—NHC(O)—CH₂—, and R⁷—C(O)(CH₂)₂—C(O)—; and R⁶ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair (e.g., R⁶ is selected from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH).

In some embodiments of formula (V′) (i.e., R⁵ is attached to the C-end of the POXZ copolymer), R⁵ is selected from the group consisting of R⁴C(O)NH—, R⁷S—, R⁷S(O)₂—, and R⁷NH-(3,4-dioxocyclobut-1-en-1,2-diyl)-NH—; and/or R⁶ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair (e.g., R⁶ is C₁₋₃ alkyl optionally substituted with one substituent selected from the group consisting of —COOH, —COOCH₃, and —C(O)NH(CH₂)₂NH₂).

In some embodiments of formula (V′), (i.e., R⁵ is attached to the C-end of the POXZ copolymer), R⁵ is R⁷—C(O)(CH₂)₂C(O)NH— or R⁷N—; and R⁶ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair (e.g., R³ is C₁₋₃ alkyl optionally substituted with one substituent selected from the group consisting of —COOH, —COOCH₃, and —C(O)NH(CH₂)₂NH₂).

In some embodiments, the conjugate has one of the following formulas (VI), (VI′), (VII), or (VII′):

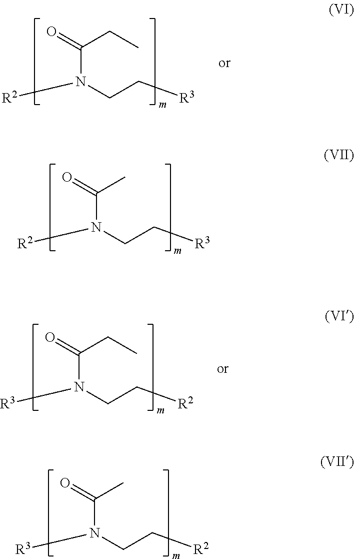

wherein R², R³, and m are as specified above, in particular with respect to any of the formulas (II), (II′), (IIa), (IIa′), (IIc), (IId), (IIe′), (IIf), (IIg′), (IIh′), (IIi′), (IIj′), (III), (III′), (Illa), and (Illa′) (and optionally (IIe)).

In any of the above embodiments of formulas (VI), (VI′), (VII), and (VII′), m preferably is 10 to 100, such as 20 to 80, 30 to 70, or 40 to 50, e.g., 20 to 25 or 45 to 50.

In some embodiments, the conjugate has one of the following formulas (VIa), (Vla′), (VIIa), or (VIIa′):

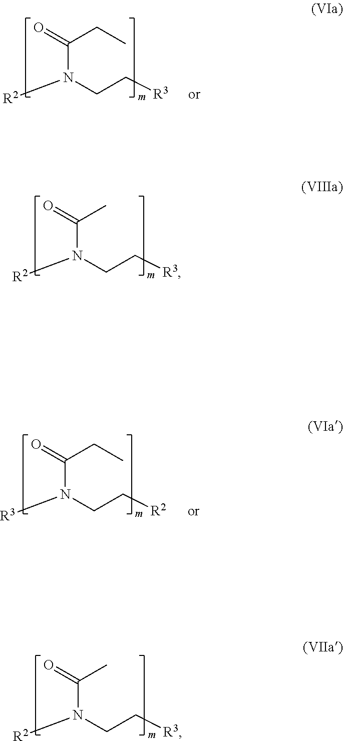

wherein m is either 20 to 25 or 45 to 50; and R² and R³ are as specified above, in particular with respect to any of the formulas (II), (II′), (IIa), (IIa′), (IIc), (IId), (IIe′), (IIff), (IIg′), (IIh′), (IIi′), (IIj′), (III), (III′), (Illa), and (Illa′) (and optionally (IIe)).

In any of the above embodiments of formulas (VI), (VIa), (VII), and (VIIa), R² is preferably selected from the group consisting of -L¹R⁴, —(CH₂)—CH(OC(O)R⁴)(CH₂OC(O)R⁴), —(CH₂)—CH(SR⁴)₂, and —(CH₂)—CH(SR⁴)—CH₂(SR⁴), wherein each R⁴ is independently a straight hydrocarbyl group having at least 10 carbon atoms (and preferably up to 30 carbon atoms, such as up to 24 carbon atoms, e.g., having 10 to 16 carbon atoms such as 10 to 14 carbon atoms); and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L¹ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴. Thus, in any of the above embodiments of formulas (VI), (VIa), (VII), and (VIIa), R² may be selected from the group consisting of R⁴—NHC(O)—(CH₂)—, R⁴—NHC(O)—(CH₂)₂—, —(CH₂)—CH(OC(O)R⁴)(CH₂OC(O)R⁴), —(CH₂)—CH(SR⁴)—CH₂(SR⁴), R⁴S—(CH₂)₃—, R⁴S(O)₂—(CH₂)₃—, and R⁴—OC(O)—(CH₂)—.

Alternatively, in any of the above embodiments of formulas (VI), (VIa), (VII), and (VIIa), R² is preferably selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—.

In any of the above embodiments of formulas (VI′), (VIa′), (VII′), and (VIIa′), R² is preferably selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein each R⁴ is independently a straight hydrocarbyl group having at least 10 carbon atoms (and preferably up to 30 carbon atoms, such as up to 24 carbon atoms, e.g., having 10 to 16 carbon atoms such as 10 to 14 carbon atoms); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—. Thus, in any of the above embodiments of formulas (VI′), (VIa′), (VII′), and (VIIa′), R² may be selected from the group consisting of R⁴C(O)NH—, R⁴S—, R⁴S(O)₂—, and R⁴NH-(3,4-dioxocyclobut-1-en-1,2-diyl)-NH—.

Alternatively, in any of the above embodiments of formulas (VI′), (VIa′), (VII′), and (VIIa′), R² preferably is R⁴—C(O)(CH₂)₂C(O)NH— or R⁴N—.

In any of the above embodiments of formulas (VI), (VI′), (VIa), (VIa′), (VII), (VII′), (VIIa), and (VIIa′), R³ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In any of the above embodiments of formulas (VI), (VIa), (VII), and (VIIa), R³ is preferably selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.

In any of the above embodiments of formulas (VI′), (VIa′), (VII′), and (VIIa′), R³ is preferably C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, more preferably R³ is C₁₋₃ alkyl optionally substituted with one substituent selected from the group consisting of —COOH, —COOCH₃, and —C(O)NH(CH₂)₂NH₂.

In any of the above embodiments of formulas (VI), (VIa), (VII), and (VIIa), it is preferred that:
R² is selected from the group consisting of -L¹R⁴, —(CH₂)—CH(OC(O)R⁴)(CH₂OC(O)R⁴), —(CH₂)—CH(SR⁴)₂, and —(CH₂)—CH(SR⁴)—CH₂(SR⁴), wherein each R⁴ is independently a straight hydrocarbyl group having at least 10 carbon atoms (and preferably up to 30 carbon atoms, such as up to 24 carbon atoms, or up to 16 carbon atoms, such as up to 15, 14, 13, 12, 11, or 10 carbon atoms, e.g., having 10 to 16 carbon atoms, such as 10 to 15 or 10 to 14 carbon atoms); and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L¹ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴; and
R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.

Alternatively, in any of the above embodiments of formulas (VI), (VIa), (VII), and (VIIa), it is preferred that:
R² is selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—; and
R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.

In any of the above embodiments of formulas (VI′), (VIa′), (VII′), and (VIIa′), it is preferred that:
R² is selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein each R⁴ is independently a straight hydrocarbyl group having at least 10 carbon atoms (and preferably up to 30 carbon atoms, such as up to 24 carbon atoms, e.g., having 10 to 16 carbon atoms such as 10 to 14 carbon atoms); the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; and
R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, preferably R³ is C₁₋₃ alkyl optionally substituted with one substituent selected from the group consisting of —COOH, —COOCH₃, and —C(O)NH(CH₂)₂NH₂.

Alternatively, in any of the above embodiments of formulas (VI′), (VIa′), (VII′), and (VIIa′), it is preferred that:
R² is R⁴—C(O)(CH₂)₂C(O)NH— or R⁴N—; and
R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, preferably R³ is C₁₋₃ alkyl optionally substituted with one substituent selected from the group consisting of —COOH, —COOCH₃, and —C(O)NH(CH₂)₂NH₂.

In some embodiments, the conjugate has one of the following formulas (VIII), (VIII′), (IX), or (IX′):

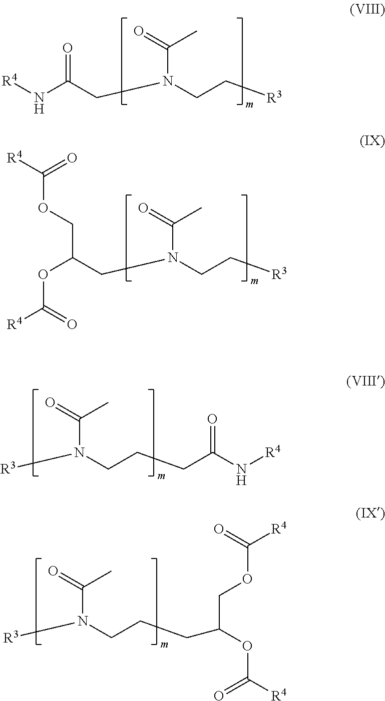

wherein R³, R⁴, and m are as specified above, in particular with respect to any of the formulas (II), (II′), (IIa), (IIa′), (III), (III′), (IIIa), and (IIIa′). In some embodiments of formula (VIII) or (VIII′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (IX) or (IX′), R⁴ is independently a straight hydrocarbyl group having at least 10 carbon and up to 30 carbon atoms, such as up to 24, 22, or 20 carbon atoms, e.g., having 10 to 16 carbon atoms, such as 10 to 15 or 10 to 14 carbon atoms.

In some embodiments, the conjugate has one of the following formulas (XV), (XV′), (XVI), (XVII), (XVII′), (XVIII), (XVIII′), (XIX), (XIX′), (XX′), (XXI′), (XXII′), or (XXIII′):

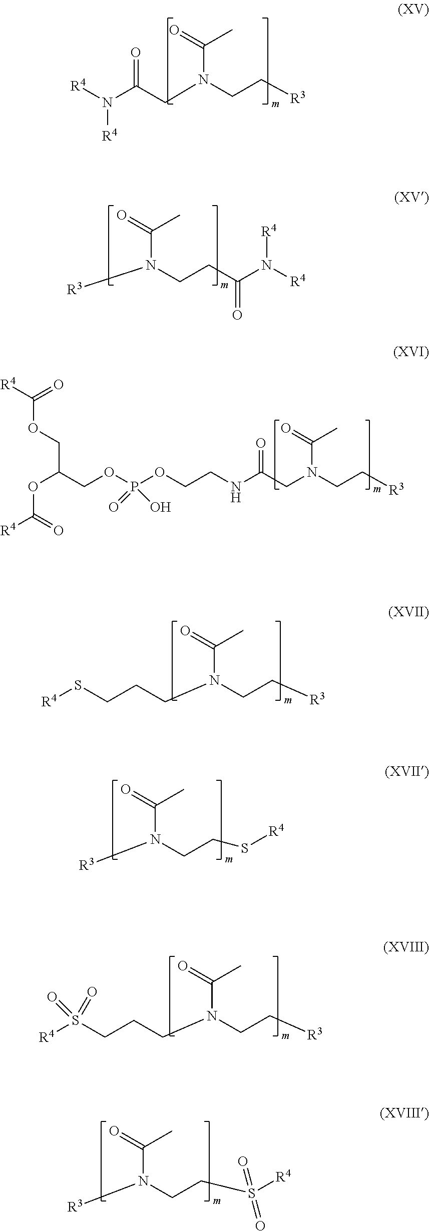

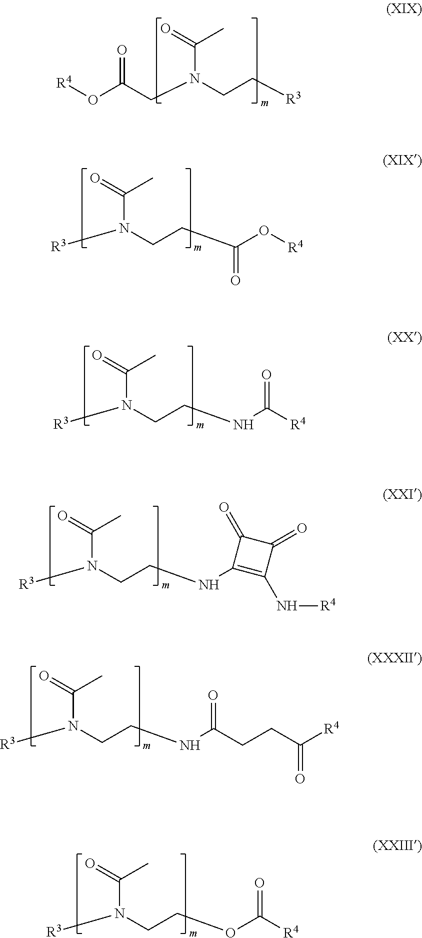

wherein R³, R⁴, and m are as specified above, in particular with respect to any of the formulas (II), (II′), (IIa), (IIa′), (III), (III′), (Illa), and (Illa′). In some embodiments of formula (XV) or (XV′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XVI), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XVII) or (XVII′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XVIII) or (XVIII′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XIX) or (XIX′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XX′) or (XXI′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XXII′) or (XXIII′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formula (XXII′), R⁴ is a tocopherol moiety, e.g., α-tocopheryl, β-tocopheryl, γ-tocopheryl, or δ-tocopheryl moiety, preferably an α-tocopheryl moiety.

In any of the above embodiments of formulas (VIII), (VIII′), (IX), or (IX′), m preferably is 10 to 100, such as 20 to 80, 30 to 70, or 40 to 50, e.g., 20 to 25 or 45 to 50.

In any of the above embodiments of formulas (XV), (XV′), (XVI), (XVII), (XVII′), (XVIII), (XVIII′), (XIX), (XIX′), (XX′), (XXI′), (XXII′), or (XXIII′), m preferably is 10 to 100, such as 20 to 80, 30 to 70, or 40 to 50, e.g., 20 to 25 or 45 to 50.

In some embodiments, the conjugate has one of the following formulas (VIIIa) or (IXa):

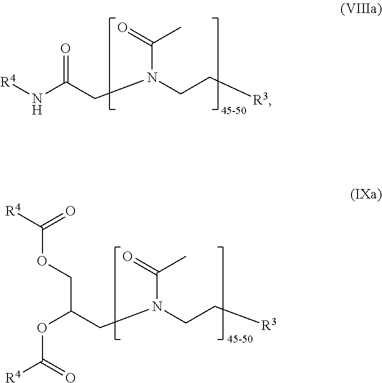

wherein R³, R⁴, and m are as specified above, in particular with respect to any of the formulas (II), (II′), (IIa), (IIa′), (III), (III′), (IIIa), and (IIIa′).

In some embodiments of formulas (XV), (XV′), (XVI), (XVII), (XVII′), (XVIII), (XVIII′), (XIX), (XIX′), (XX′), (XXI′), (XXII′), and (XXIII′), m is 20 to 25.

In some embodiments of formulas (XV), (XV′), (XVI), (XVII), (XVII′), (XVIII), (XVIII′), (XIX), (XIX′), (XX′), (XXI′), (XXII′), and (XXIII′), m is 45 to 50.

In any of the above embodiments of formulas (VIII), (VIII′), (VIIIa), (IX), (IX′), and (IXa), each R⁴ is preferably independently a straight hydrocarbyl group having at least 10 carbon atoms (and preferably up to 30 carbon atoms, such as up to 24 carbon atoms, e.g., having 10 to 16 carbon atoms). In some embodiments of formulas (VIII), (VIII′), and (VIIIa), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formulas (IX), (IX′), and (IXa), R⁴ is independently a straight hydrocarbyl group having at least 10 carbon and up to 30 carbon atoms, such as up to 24, 22, or 20 carbon atoms, e.g., having 10 to 16 carbon atoms, such as 10 to 15 or 10 to 14 carbon atoms.

In any of the above embodiments of formulas (XV), (XV′), (XVI), (XVII), (XVII′), (XVIII), (XVIII′), (XIX), (XIX′), (XX′), (XXI′), (XXII′), and (XXIII′), each R⁴ is preferably independently a straight hydrocarbyl group having at least 10 carbon atoms (and preferably up to 30 carbon atoms, such as up to 24 carbon atoms, e.g., having 10 to 16 carbon atoms). In some embodiments of formulas (XV), (XV′), (XVI), (XVII), (XVII′), (XVIII), (XVIII′), (XIX), (XIX′), (XX′), (XXI′), (XXII′), and (XXIII′), R⁴ is a straight hydrocarbyl group having at least 10 carbon and up to 16 carbon atoms, such as up to 15, 14, 13, 12, 11, or 10 carbon atoms, e.g., having 10 to 16 carbon atoms, such as 10 to 15 or 10 to 14 carbon atoms. In some embodiments of formulas (XV), (XV′), (XVI), (XVII), (XVII′), (XVIII), (XVIII′), (XIX), (XIX′), (XX′), (XXI′), (XXII′), and (XXIII′), R⁴ is independently a straight hydrocarbyl group having at least 10 carbon and up to 30 carbon atoms, such as up to 24, 22, or 20 carbon atoms, e.g., having 10 to 16 carbon atoms, such as 10 to 15 or 10 to 14 carbon atoms. In some embodiments of formula (XXII′), R⁴ is a tocopherol moiety, e.g., α-tocopheryl, β-tocopheryl, γ-tocopheryl, or δ-tocopheryl moiety, preferably an α-tocopheryl moiety.

In any of the above embodiments of formulas (VIII), (VIII′), (VIIIa), (IX), (IX′), and (IXa), R³ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In any of the above embodiments of formulas (XV), (XV′), (XVI), (XVII), (XVII′), (XVIII), (XVIII′), (XIX), (XIX′), (XX′), (XXI′), (XXII′), and (XXIII′), R³ is preferably selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂. —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In any of the above embodiments of formulas (VIII), (VIII′), (VIIIa), (IX), (IX′), and (IXa), it is preferred that:
each R⁴ is independently a straight hydrocarbyl group having at least 10 carbon atoms (and preferably up to 30 carbon atoms, such as up to 24 carbon atoms, e.g., having 10 to 16 carbon atoms, such as 10 to 14 carbon atoms); and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In this respect, for formulas (VIII′) and (IX′), it is preferred that R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, more preferably R³ is C₁₋₃ alkyl optionally substituted with one substituent selected from the group consisting of —COOH, —COOCH₃, and —C(O)NH(CH₂)₂NH₂.

In any of the above embodiments of formulas (XV), (XV′), (XVI), (XVII), (XVII′), (XVIII), (XVIII′), (XIX), (XIX′), (XX′), (XXI′), (XXII′), and (XXIII′), it is preferred that:
each R⁴ is independently a straight hydrocarbyl group having at least 10 carbon atoms (and preferably up to 30 carbon atoms, such as up to 24 carbon atoms, e.g., having 10 to 16 carbon atoms, such as 10 to 14 carbon atoms); and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In this respect, for formulas (XV′), (XVII′), (XVIII′), (XIX′), (XX′), (XXI′), (XXII′), and (XXIII′), it is preferred that R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, more preferably R³ is C₁₋₃ alkyl optionally substituted with one substituent selected from the group consisting of —COOH, —COOCH₃, and —C(O)NH(CH₂)₂NH₂.

In any of the above embodiments of formulas (VIII), (VIII′), and (VIIIa), it is preferred that:
each R⁴ is independently a straight hydrocarbyl group having at least 10 carbon atoms (and preferably up to 16 carbon atoms, such as up to 15, 14, 13, 12, 11, or 10 carbon atoms, e.g., having 10 to 16 carbon atoms, such as 10 to 15 or 10 to 14 carbon atoms); and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In this respect, for formula (VIII′), it is preferred that R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, more preferably R³ is C₁₋₃ alkyl optionally substituted with one substituent selected from the group consisting of —COOH, —COOCH₃, and —C(O)NH(CH₂)₂NH₂.

In any of the above embodiments of formulas (IX), (IX′), and (IXa), it is preferred that:
each R⁴ is independently a straight hydrocarbyl group having at least 10 carbon atoms (and preferably up to 30 carbon atoms, such as up to 24 carbon atoms, e.g., having 10 to 15 carbon atoms); and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.

In this respect, for formula (IX′), it is preferred that R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, more preferably R³ is C₁₋₃ alkyl optionally substituted with one substituent selected from the group consisting of —COOH, —COOCH₃, and —C(O)NH(CH₂)₂NH₂.

Particular examples of the conjugate of (a) a POX and/or polyoxazine POZ polymer and (b) one or more hydrophobic chains are the following compounds (II-1) to (II-34) and (II′-35) to (II′-48) and compounds (II-49), (II-50), (II′-51), (II′-52), and (II-53) to (II-56):

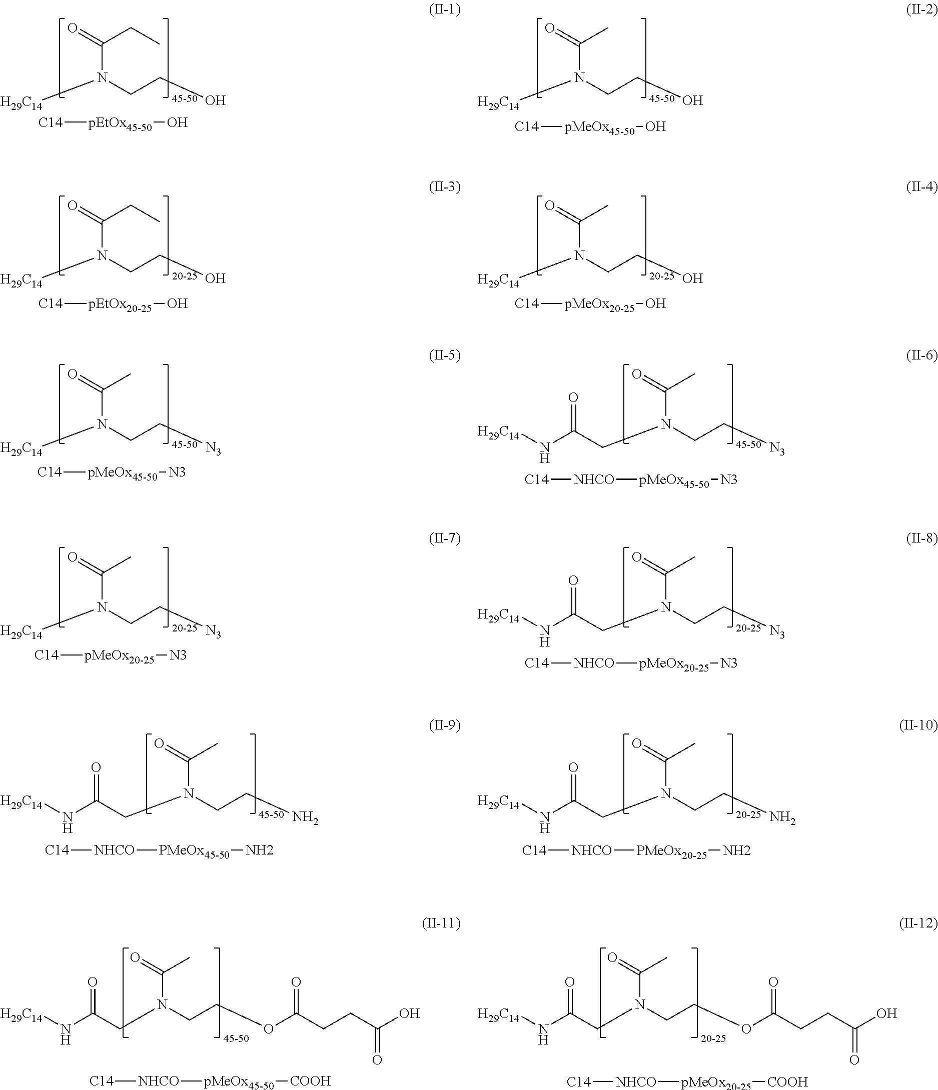

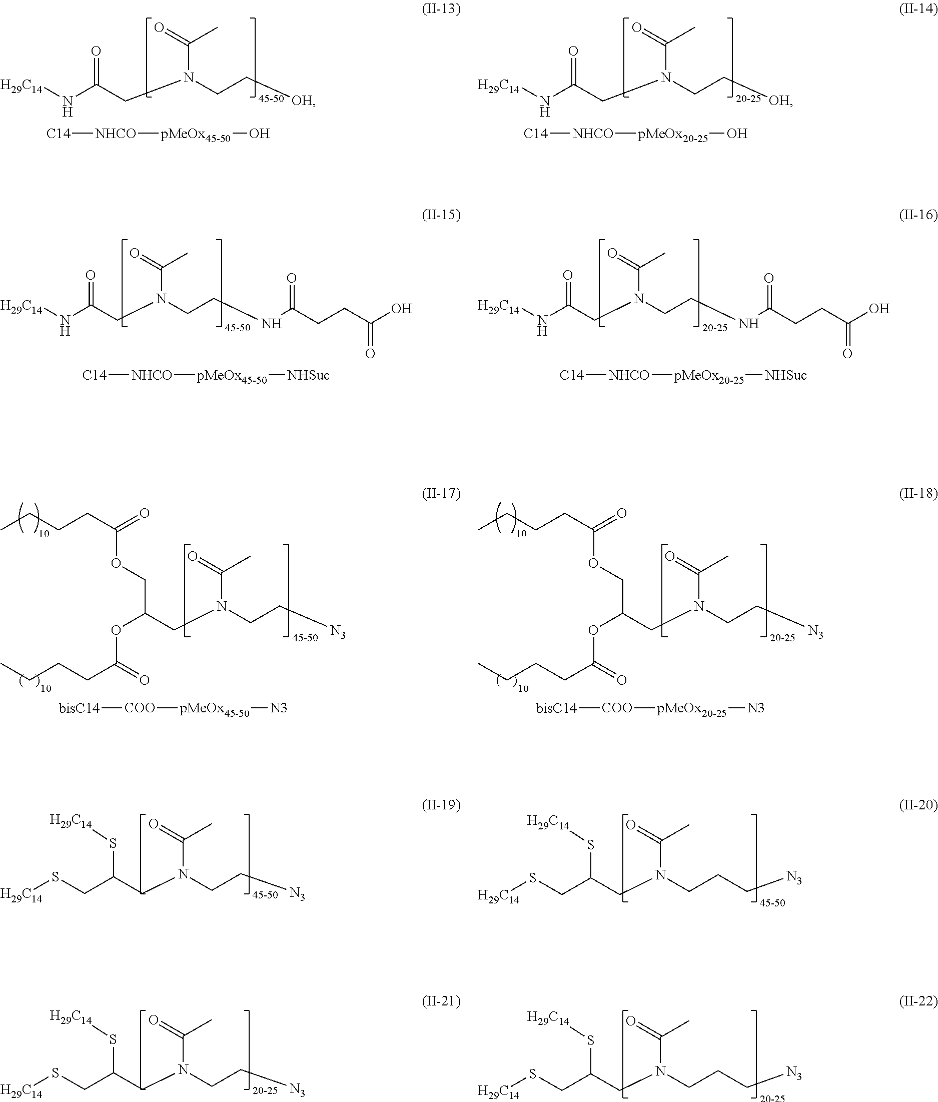

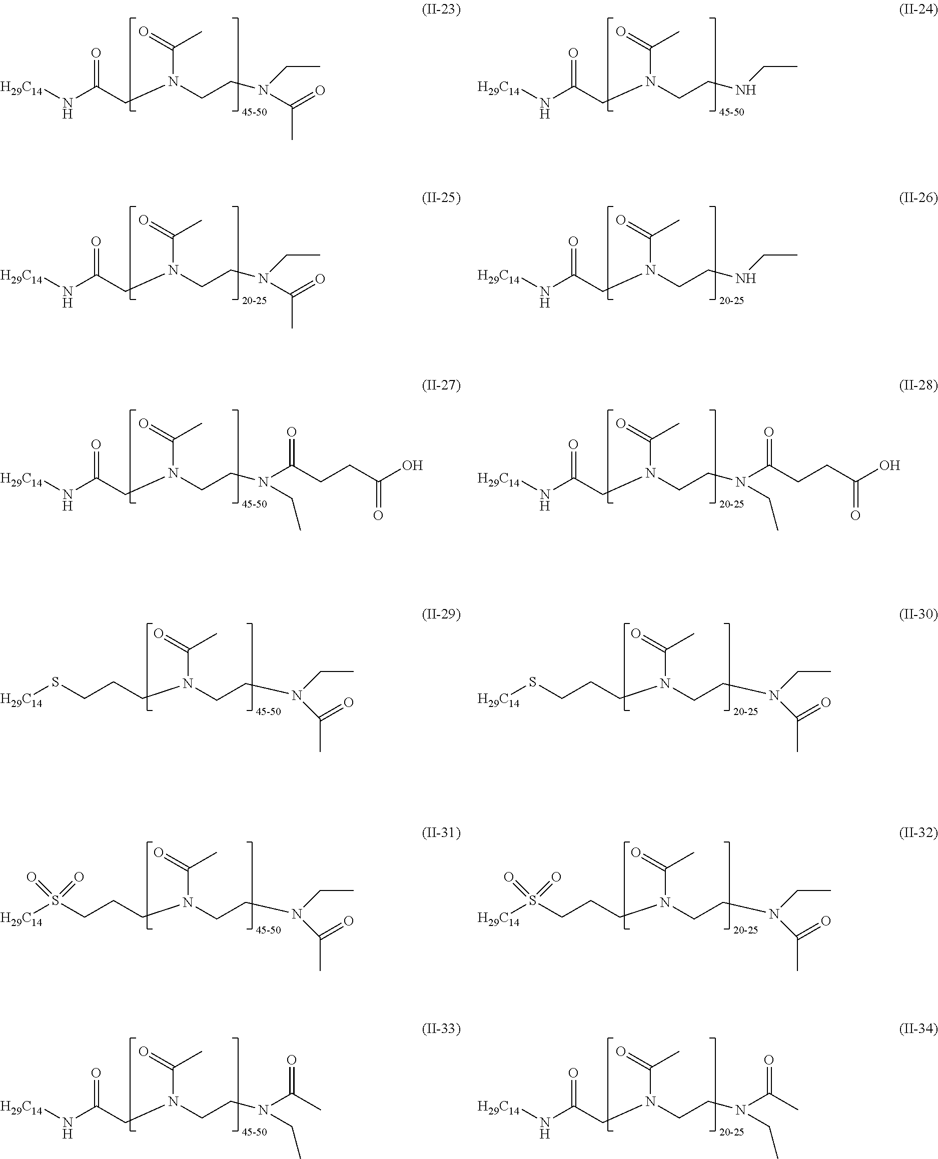

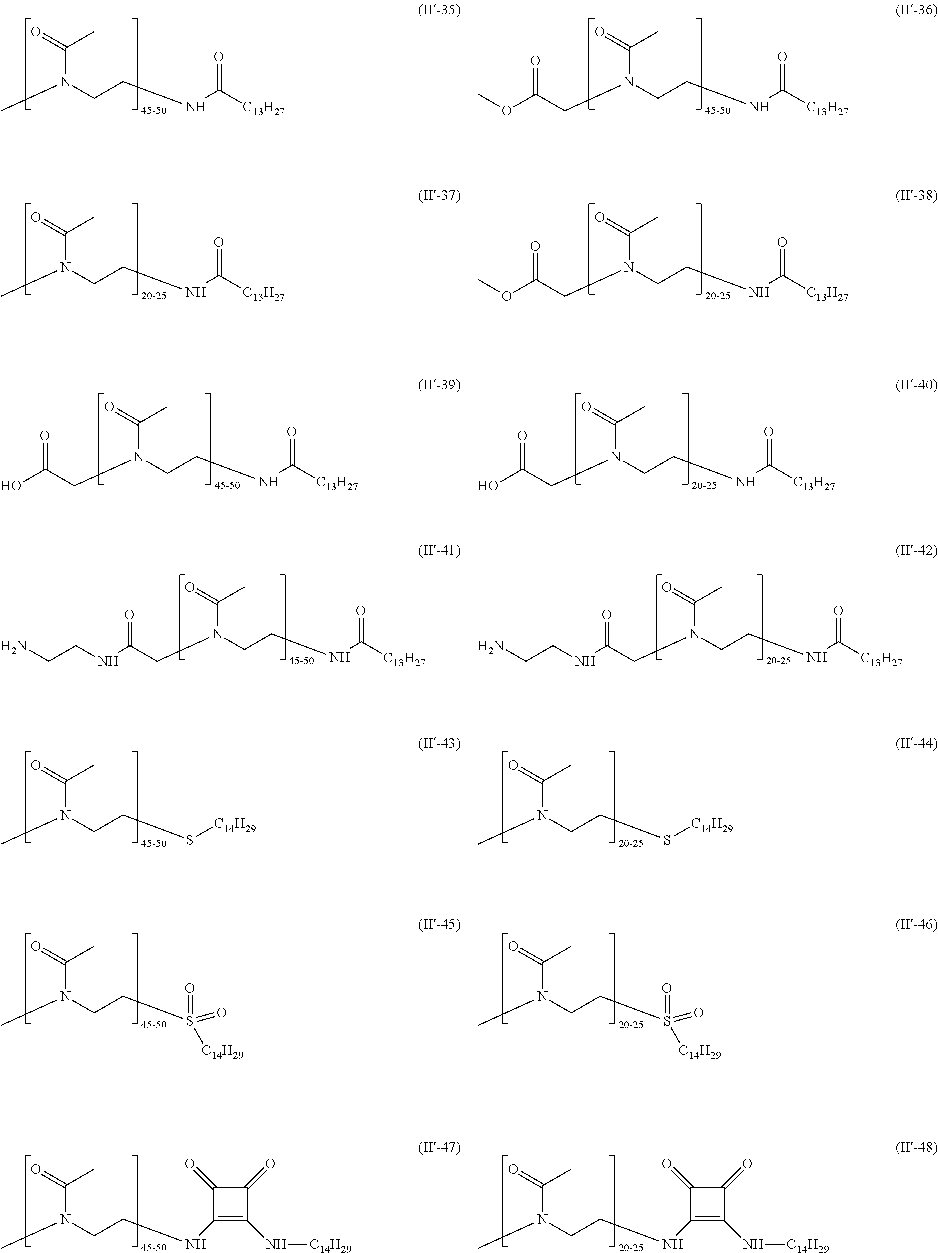

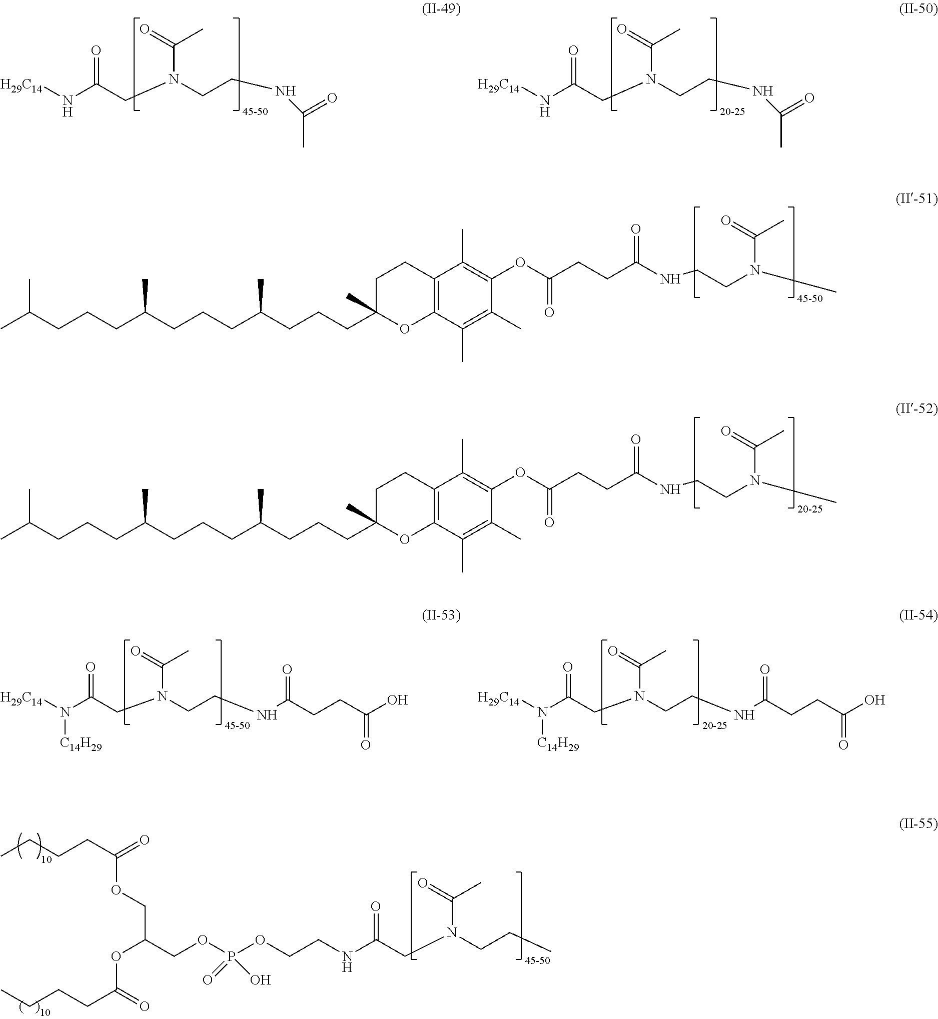

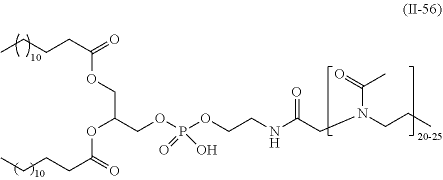

wherein in each case C₁₄H₂₉ refers to the moiety-(CH₂)₁₃CH₃, and in each case C₁₃H₂₇ refers to the moiety-(CH₂)₁₂CH₃.

It is to be understood that any reference to a conjugate of (a) a POX and/or POZ polymer and (b) one or more hydrophobic chains (in particular, to a conjugate of any one of formulas (IIa), (IIa′), (IIb), (IIb′), (IIc), (IId), (IIe′), (IIf), (IIg′), (IIh′), (IIi′), (IIj′), (III), (III′), (IIIa), (Illa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or to a conjugate of any one of formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)) also includes the salts (in particular pharmaceutically acceptable salts), tautomers, stereoisomers, solvates (e.g., hydrates), and isotopically labeled forms thereof.

In certain instances, the conjugate of (a) a POX and/or POZ polymer and (b) one or more hydrophobic chains (in particular, a conjugate of any one of formulas (IIa), (IIa′), (IIb), (IIb′), (IIc), (IId), (IIe′), (IIf), (IIg′), (IIh′), (IIi′), (IIj′), (III), (III′), (IIIa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (VIa′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or a conjugate of any one of formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)) may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipid present in the particle (in particular, of the total lipid and lipid-like material present in the LNP).

### Additional Lipids

Nucleic acid (such as DNA or RNA) compositions (such as nucleic acid (e.g., DNA or RNA) particles) described herein may also comprise one or more additional lipids (including lipid-like materials), i.e., lipids other than cationic or cationically ionizable lipids (also collectively referred to herein as cationic lipids), i.e., non-cationic lipids (including non-cationically ionizable lipids). Collectively, anionic and neutral lipids are referred to herein as non-cationic lipids. Optimizing the formulation of nucleic acid (such as DNA or RNA) compositions (such as nucleic acid (e.g., DNA or RNA) particles) by addition of other hydrophobic moieties, such as cholesterol and lipids, in addition to an ionizable/cationic lipid may enhance particle stability and efficacy of nucleic acid delivery. An additional lipid may or may not affect the overall charge of the nucleic acid (such as DNA or RNA) compositions (such as of the nucleic acid (e.g., DNA or RNA) particles).

In certain embodiments, the additional lipid is a non-cationic lipid. The non-cationic lipid may comprise, e.g., one or more anionic lipids and/or neutral lipids. As used herein, a “neutral lipid” refers to any of a number of lipid species that exist either in an uncharged or neutral zwitterionic form at a selected pH. In certain embodiments, the additional lipid is a phospholipid (such as DOPE) and/or a steroid (e.g., a sterol such as cholesterol); in particular embodiments, the additional lipid is a mixture of a a phospholipid (such as DOPE) and a steroid (e.g., a sterol such as cholesterol). In certain embodiments, the additional lipid comprises one of the following neutral lipid components: (1) a phospholipid, (2) cholesterol or a derivative thereof; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, tocopherol and derivatives thereof, and mixtures thereof.

Specific phospholipids that can be used include, but are not limited to, phosphatidylcholines, phosphatidylethanolamines, phosphatidylglycerols, phosphatidic acids, phosphatidylserines or sphingomyelin. Such phospholipids include in particular diacylphosphatidylcholines, such as distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₋₆ Lyso PC) and phosphatidylethanolamines, in particular diacylphosphatidylethanolamines, such as dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), diphytanoyl-phosphatidylethanolamine (DPyPE), and further phosphatidylethanolamine lipids with different hydrophobic chains.

In certain embodiments, the additional lipid is DOPE or DOPE and cholesterol.

In certain embodiments, the nucleic acid (such as DNA or RNA) compositions (such as nucleic acid (e.g., DNA or RNA) particles) include both a cationic lipid and an additional lipid. In an exemplary embodiment, the cationic lipid is DODMA and the additional lipid is DOPE or DOPE and cholesterol.

Without wishing to be bound by theory, the amount of the cationic or cationically ionizable lipid compared to the amount of the at least one additional lipid may affect important characteristics of the nucleic acid (such as DNA or RNA) compositions (such as of the nucleic acid (e.g., DNA or RNA) particles), such as charge, particle size, stability, tissue selectivity, and bioactivity of the nucleic acid (such as DNA or RNA). Accordingly, in some embodiments, the molar ratio of the cationic or cationically ionizable lipid to the at least one additional lipid is from about 10:0 to about 1:9, about 4:1 to about 1:2, or about 3:1 to about 1:1.

In some embodiments, the non-cationic lipid, in particular neutral lipid, (e.g., one or more phospholipids and/or cholesterol) may comprise from about 0 mol % to about 90 mol %, from about 20 mol % to about 80 mol %, from about 25 mol % to about 75 mol %, from about 30 mol % to about 70 mol %, from about 35 mol % to about 65 mol %, or from about 40 mol % to about 60 mol %, of the total lipid present in the composition (such as of the total lipid present in the particle, in particular, of the total lipid and lipid-like material present in the LNP).

In certain preferred embodiments, the non-cationic lipid, in particular neutral lipid, comprises a phospholipid such as DOPE of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipid present in the composition (such as of the total lipid present in the particle, in particular, of the total lipid and lipid-like material present in the LNP).

In certain preferred embodiments, the non-cationic lipid, in particular neutral lipid, comprises cholesterol or a derivative thereof of from about 10 mol % to about 80 mol %, from about 10 mol % to about 70 mol %, or from about 10 mol % to about 60 mol %, such as from about 15 mol % to about 65 mol %, from about 20 mol % to about 60 mol %, from about 25 mol % to about 55 mol %, or from about 30 mol % to about 50 mol % of the total lipid present in the composition (such as of the total lipid present in the particle, in particular, of the total lipid and lipid-like material present in the LNP).

In certain preferred embodiments, the non-cationic lipid, in particular neutral lipid, comprises a mixture of: (i) a phospholipid such as DOPE of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipid present in the composition (such as of the total lipid present in the particle); and (ii) cholesterol or a derivative thereof such as cholesterol of from about 10 mol % to about 80 mol %, from about 10 mol % to about 70 mol %, or from about 10 mol % to about 60 mol %, such as from about 15 mol % to about 65 mol %, from about 20 mol % to about 60 mol %, from about 25 mol % to about 55 mol %, or from about 30 mol % to about 50 mol % of the total lipid present in the composition (such as of the total lipid present in the particle, in particular, of the total lipid and lipid-like material present in the LNP). As a non-limiting example, a lipid composition (such as a lipid particle) comprising a mixture of a phospholipid and cholesterol may comprise DOPE of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipid present in the composition (such as of the total lipid present in the particle) and cholesterol of from about 10 mol % to about 80 mol %, from about 10 mol % to about 70 mol %, or from about 10 mol % to about 60 mol %, such as from about 15 mol % to about 65 mol %, from about 20 mol % to about 60 mol %, from about 25 mol % to about 55 mol %, or from about 30 mol % to about 50 mol % of the total lipid present in the composition (such as of the total lipid present in the particle, in particular, of the total lipid and lipid-like material present in the LNP).

### Embodiments of Nucleic Acid (Such as DNA or RNA)-Lipid Compositions (e.g., of Nucleic Acid (Such as DNA or RNA)-Lipid Nanoparticles)

In some embodiments, the nucleic acid (such as DNA or RNA)-lipid compositions (e.g., nucleic acid (such as DNA or RNA)-lipid particles, in particular RNA LNPs) in addition to nucleic acid (such as DNA or RNA) comprise (i) a cationic or cationically ionizable lipid which may comprise from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipid present in the composition (such as of the total lipid present in the particle, in particular, of the total lipid and lipid-like material present in the LNP), (ii) a non-cationic lipid, in particular neutral lipid, (e.g., one or more phospholipids and/or cholesterol) which may comprise from about 0 mol % to about 90 mol %, from about 20 mol % to about 80 mol %, from about 25 mol % to about 75 mol %, from about 30 mol % to about 70 mol %, from about 35 mol % to about 65 mol %, or from about 40 mol % to about 60 mol %, of the total lipid present in the composition (such as of the total lipid present in the particle, in particular, of the total lipid and lipid-like material present in the LNP), and (iii) a conjugate of (a) a POX and/or POZ polymer and (b) one or more hydrophobic chains (in particular, a conjugate of any one of formulas (IIa), (IIa′), (IIb), (IIb′), (IIc), (IId), (IIe′), (IIf), (IIg′), (IIh′), (IIi′), (IIj′), (III), (III′), (Illa), (IIIa′), (IIIb), (IIIb′), (IV), (IV′), (IVa), (IVb), (IVc′), (IVd′), (IVe′), (IVf), (IVg′), (IVh′), (V), (V′), (VI), (VI′), (VIa), (Vla′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or a conjugate of any one of formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)) which may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipid present in the composition (such as of the total lipid present in the particle, in particular, of the total lipid and lipid-like material present in the LNP).

In some embodiments, the cationic or cationically ionizable lipid is a compound selected from DODMA, DOTMA, DPL14, 3D-P-DMA, Formula (X), and Formula (XI), (e.g., any one of Formulas (XIIa), (XIIb), (XIIIa), (XIIIb), (XIV-1), (XIV-2), and (XIV-3)).

In some embodiments, cationic or cationically ionizable lipid is DODMA.

In some embodiments, the cationic or cationically ionizable lipid is DOTMA.

In some embodiments, the cationic or cationically ionizable lipid is DPL14.

In some embodiments, the cationic or cationically ionizable lipid is 3D-P-DMA.

In some embodiments, the cationic or cationically ionizable lipid is a compound of Formula (X).

In some embodiments, the cationic or cationically ionizable lipid is a compound of Formula (XI), e.g., any one of Formulas (XIIa), (XIIb), (XIIIa), (XIIIb), (XIV-1), (XIV-2), and (XIV-3).

In some embodiments, the neutral lipid is a phospholipid. In some embodiments, the phospholipid is DOPE.

In some embodiments, the neutral lipid is cholesterol.

In some embodiments, the neutral lipid is a combination of a phospholipid and cholesterol. In some embodiments, the phospholipid is DOPE.

In some embodiments, the nucleic acid is RNA, in particular a modified RNA. In some embodiments, the RNA is a vaccine RNA.

In certain preferred embodiments, the non-cationic lipid, in particular neutral lipid, comprises a phospholipid of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipid present in the composition (such as of the total lipid present in the particle, in particular, of the total lipid and lipid-like material present in the LNP).

In certain preferred embodiments, the non-cationic lipid, in particular neutral lipid, comprises cholesterol or a derivative thereof of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipid present in the composition (such as of the total lipid present in the particle, in particular, of the total lipid and lipid-like material present in the LNP).

In certain preferred embodiments, the non-cationic lipid, in particular neutral lipid, comprises a mixture of: (i) a phospholipid such as DOPE of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipid present in the composition (such as of the total lipid present in the particle, in particular, of the total lipid and lipid-like material present in the LNP); and (ii) cholesterol or a derivative thereof such as cholesterol of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipid present in the composition (such as of the total lipid present in the particle, in particular, of the total lipid and lipid-like material present in the LNP). As a non-limiting example, a lipid composition (such as a lipid particle, in particular RNA LNP) comprising a mixture of a phospholipid and cholesterol may comprise DOPE of from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipid present in the composition (such as of the total lipid present in the particle, in particular, of the total lipid and lipid-like material present in the LNP) and cholesterol of from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipid present in the composition (such as of the total lipid present in the particle, in particular, of the total lipid and lipid-like material present in the LNP).

Typically, the POX and/or POZ polymer of the conjugate (in particular, of a conjugate of any one of formulas (IIa), (IIa′), (IIb), (IIb), (IIc), (IId), (IIe′), (IIf), (IIg), (IIh′), (IIi′), (IIj′), (III), (III′), (IIIa), (Illa′), (IIIb), (IIIb′), (IV), (IV′), (IVa), (IVb), (IVc′), (IVd′), (IVe′), (IVf), (IVg′), (IVh′), (V), (V′), (VI), (VI′), (VII), (VII′), (VIII), (VIII′), (IX), (IX′) or of a conjugate of any one of formulas (IIe), (IVc), (XV), (XV′), (XVI), (XVII), (XVII′), (XVIII), (XVIII′), (XIX), (XIX′), (XX′), (XXI′), (XXII′), and (XXIII′)) has between 2 and 200, between 5 and 200, between 5 and 190, between 5 and 180, between 5 and 170, between 5 and 160, between 5 and 150, between 5 and 140, between 5 and 130, between 5 and 120, between 5 and 110, between 5 and 100, between 5 and 90, between 5 and 80, between 10 and 200, between 10 and 190, between 10 and 180, between 10 and 170, between 10 and 160, between 10 and 150, between 10 and 140, between 10 and 130, between 10 and 120, between 10 and 110, between 10 and 100, between 10 and 90, between 10 and 80, between 10 and 50, between 10 and 30, or between 10 and 25 POX and/or POZ repeating units.

In some embodiments, the nucleic acid (such as DNA or RNA)-lipid compositions (e.g., nucleic acid (such as DNA or RNA)-lipid particles, in particular RNA LNPs) in addition to nucleic acid (such as DNA or RNA) comprise (i) DODMA which may comprise from about 10 mol % to about 80 mol %, from about 20 mol % to about 60 mol %, from about 25 mol % to about 55 mol %, from about 30 mol % to about 50 mol %, from about 35 mol % to about 45 mol %, or about 40 mol % of the total lipid present in the composition (such as of the total lipid present in the particle, in particular, of the total lipid and lipid-like material present in the LNP), (ii) DOPE which may comprise from about 5 mol % to about 50 mol %, from about 5 mol % to about 45 mol %, from about 5 mol % to about 40 mol %, from about 5 mol % to about 35 mol %, from about 5 mol % to about 30 mol %, from about 5 mol % to about 25 mol %, or from about 5 mol % to about 20 mol % of the total lipid present in the composition (such as of the total lipid present in the particle, in particular, of the total lipid and lipid-like material present in the LNP), (iii) cholesterol which may comprise from about 10 mol % to about 80 mol %, from about 10 mol % to about 70 mol %, from about 15 mol % to about 65 mol %, from about 20 mol % to about 60 mol %, from about 25 mol % to about 55 mol %, or from about 30 mol % to about 50 mol % of the total lipid present in the composition (such as of the total lipid present in the particle, in particular, of the total lipid and lipid-like material present in the LNP); and (iv) a conjugate of (a) a POX and/or POZ polymer and (b) one or more hydrophobic chains (in particular, a conjugate of any one of formulas (IIa), (IIa), (IIb), (IIb), (IIc), (IId), (IIe), (IIf), (IIg), (IIh), (IIi′), (IIj′), (III), (III′), (IIIa), (IIIa′), (IIIb), (IIIb), (IV), (IV′), (IVa), (IVb), (IVc), (IVd′), (IVe′), (IVf), (IVg′), (IVh), (V), (V′), (VI), (VI), (VIa), (VIa), (VII), (VII′), (VIIa), (VIIa), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), and (II′-48), or a conjugate of any one of formulas (IIc), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), and (II-56)) which may comprise from about 0.2 mol % to about 50 mol %, from about 0.25 mol % to about 30 mol %, from about 0.5 mol % to about 25 mol %, from about 0.75 mol % to about 25 mol %, from about 1 mol % to about 25 mol %, from about 1 mol % to about 20 mol %, from about 1 mol % to about 15 mol %, from about 1 mol % to about 10 mol %, from about 1 mol % to about 5 mol %, from about 1.5 mol % to about 25 mol %, from about 1.5 mol % to about 20 mol %, from about 1.5 mol % to about 15 mol %, from about 1.5 mol % to about 10 mol %, from about 1.5 mol % to about 5 mol %, from about 2 mol % to about 25 mol %, from about 2 mol % to about 20 mol %, from about 2 mol % to about 15 mol %, from about 2 mol % to about 10 mol %, or from about 2 mol % to about 5 mol % of the total lipid present in the composition (such as of the total lipid present in the particle, in particular, of the total lipid and lipid-like material present in the LNP).

In certain embodiments, the nucleic acid (such as DNA or RNA) compositions (e.g., nucleic acid (such as DNA or RNA) particles, in particular RNA LNPs) include a conjugate of (a) a POX and/or POZ polymer and (b) one or more hydrophobic chains according to the general formula (II), (II′), (IIa), (IIa), (IIb), (IIb), (IIc), (IId), (IIe), (IIf), (IIg), (IIh′), (IIi′), (IIj), (III), (III), (IIIa), (IIIa), (IIIb), (IIIb), (IV), (IV′), (IVa), (IVb), (IVc′), (IVd′), (IVe′), (IVf), (IVg′), (IVh′), (V), (V′), (VI), (VI′), (VIa), (VIa), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48) disclosed herein or according to the general formula (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56) disclosed herein; DODMA; DOPE; and cholesterol.

Nucleic acid (such as DNA or RNA) particles (such as RNA LNPs) described herein may be prepared by (a) preparing a nucleic acid (such as DNA or RNA) solution (e.g., an RNA solution) containing water and a buffering system (e.g., citrate at a pH of about 4-5); (b) preparing an ethanolic solution comprising the cationically ionizable lipid, the conjugate of a POX and/or POZ polymer and one or more hydrophobic chains (in particular, a conjugate of any one of formulas (IIa), (IIa′), (IIb), (IIb′), (IIc), (IId), (IIe′), (IIf), (IIg′), (IIh′), (IIi′), (IIj′), (III), (III′), (IIIa), (IIIa″), (IIIb), (IIIb′), (IV), (IVa), (IVb), (IVc′), (IVd′), (IVe), (IVf), (IVg), (IVh), (V), (V′), (VI), (VI′), (VIa), (Vla′), (VII), (VII′), (VIIa), (VIIa′), (VIII), (VIII′), (VIIIa), (IX), (IX′), (IXa), (II-1), (II-2), (II-3), (II-4), (II-5), (II-6), (II-7), (II-8), (II-9), (II-10), (II-11), (II-12), (II-13), (II-14), (II-15), (II-16), (II-17), (II-18), (II-19), (II-20), (II-21), (II-22), (II-23), (II-24), (II-25), (II-26), (II-27), (II-28), (II-29), (II-30), (II-31), (II-32), (II-33), (II-34), (II′-35), (II′-36), (II′-37), (II′-38), (II′-39), (II′-40), (II′-41), (II′-42), (II′-43), (II′-44), (II′-45), (II′-46), (II′-47), or (II′-48), or a conjugate of any one of formulas (IIe), (IVc), (XV), (XV′), (XVI), (XVII), (XVII′), (XVIII), (XVIII′), (XIX), (XIX′), (XX′), (XXI′), (XXII′), (XXIII′), (II-49), (II-50), (II′-51), (II′-52), (II-53), (II-54), (II-55), or (II-56)), and, if present, one or more additional lipids; (c) mixing the nucleic acid solution prepared under (a) with the ethanolic solution prepared under (b) (e.g., using microfluidic system (such as Nanoassembler)), thereby preparing a first intermediate formulation comprising the particles (e.g., LNPs) dispersed in a first aqueous phase; and (d) filtrating the first intermediate formulation prepared under (c) using a further aqueous solution (e.g., for buffer exchange), thereby preparing the formulation comprising particles (e.g., LNPs) dispersed in a final aqueous phase. After step (c) one or more steps selected from diluting and filtrating, such as tangential flow filtrating or diafiltrating, can follow. The formulation comprising particles (e.g., LNPs) may be stored (e.g., at about 4° C.).

Nucleic acid (such as DNA or RNA) particles (in particular RNA LNPs) described herein may have an average diameter ranging from about 30 nm to about 1000 nm, such as from about 30 nm to about 800 nm, from about 30 nm to about 700 nm, from about 30 nm to about 600 nm, from about 30 nm to about 500 nm, from about 30 nm to about 450 nm, from about 30 nm to about 400 nm, from about 30 nm to about 350 nm, from about 30 nm to about 300 nm, from about 30 nm to about 250 nm, from about 30 nm to about 200 nm, from about 30 nm to about 190 nm, from about 30 nm to about 180 nm, from about 30 nm to about 170 nm, from about 30 nm to about 160 nm, from about 30 nm to about 150 nm, from about 50 nm to about 500 nm, from about 50 nm to about 450 nm, from about 50 nm to about 400 nm, from about 50 nm to about 350 nm, from about 50 nm to about 300 nm, from about 50 nm to about 250 nm, from about 50 nm to about 200 nm, from about 50 nm to about 190 nm, from about 50 nm to about 180 nm, from about 50 nm to about 170 nm, from about 50 nm to about 160 nm, or from about 50 nm to about 150 nm.

In certain embodiments, nucleic acid (such as DNA or RNA) particles (in particular RNA LNPs) described herein have an average diameter ranging from about 40 nm to about 800 nm, such as from about 50 nm to about 700 nm, from about 60 nm to about 600 nm, from about 70 nm to about 500 nm, from about 80 nm to about 400 nm, from about 150 nm to about 800 nm, from about 150 nm to about 700 nm, from about 150 nm to about 600 nm, from about 200 nm to about 600 nm, from about 200 nm to about 500 nm, or from about 200 nm to about 400 nm.

Nucleic acid (such as DNA or RNA) particles (in particular RNA LNPs) described herein, e.g. generated by the processes described herein, may exhibit a polydispersity index less than about 0.5, such as less than about 0.4, less than about 0.3, less than about 0.2 or about 0.1 or less. By way of example, the nucleic acid (such as DNA or RNA) particles can exhibit a polydispersity index in a range of about 0.1 to about 0.3.

### Additional Treatments

In certain embodiments, additional treatments may be administered to a patient in combination with the treatments described herein. Such additional treatments include classical cancer therapy, e.g., radiation therapy, surgery, hyperthermia therapy and/or chemotherapy. Furthermore, such additional treatments include treatments involving immune checkpoint modulators.

Chemotherapy is a type of cancer treatment that uses one or more anti-cancer drugs (chemotherapeutic agents), usually as part of a standardized chemotherapy regimen. The term chemotherapy has come to connote non-specific usage of intracellular poisons to inhibit mitosis. The connotation excludes more selective agents that block extracellular signals (signal transduction). The development of therapies with specific molecular or genetic targets, which inhibit growth-promoting signals from classic endocrine hormones (primarily estrogens for breast cancer and androgens for prostate cancer) are now called hormonal therapies. By contrast, other inhibitions of growth-signals like those associated with receptor tyrosine kinases are referred to as targeted therapy.

Importantly, the use of drugs (whether chemotherapy, hormonal therapy or targeted therapy) constitutes systemic therapy for cancer in that they are introduced into the blood stream and are therefore in principle able to address cancer at any anatomic location in the body. Systemic therapy is often used in conjunction with other modalities that constitute local therapy (i.e. treatments whose efficacy is confined to the anatomic area where they are applied) for cancer such as radiation therapy, surgery or hyperthermia therapy.

Traditional chemotherapeutic agents are cytotoxic by means of interfering with cell division (mitosis) but cancer cells vary widely in their susceptibility to these agents. To a large extent, chemotherapy can be thought of as a way to damage or stress cells, which may then lead to cell death if apoptosis is initiated.

Chemotherapeutic agents include alkylating agents, antimetabolites, anti-microtubule agents, topoisomerase inhibitors, and cytotoxic antibiotics.

Alkylating agents have the ability to alkylate many molecules, including proteins, RNA and DNA. The subtypes of alkylating agents are the nitrogen mustards, nitrosoureas, tetrazines, aziridines, cisplatins and derivatives, and non-classical alkylating agents. Nitrogen mustards include mechlorethamine, cyclophosphamide, melphalan, chlorambucil, ifosfamide and busulfan. Nitrosoureas include N-Nitroso-N-methylurea (MNU), carmustine (BCNU), lomustine (CCNU) and semustine (MeCCNU), fotemustine and streptozotocin. Tetrazines include dacarbazine, mitozolomide and temozolomide. Aziridines include thiotepa, mytomycin and diaziquone (AZQ). Cisplatin and derivatives include cisplatin, carboplatin and oxaliplatin. They impair cell function by forming covalent bonds with the amino, carboxyl, sulfhydryl, and phosphate groups in biologically important molecules. Non-classical alkylating agents include procarbazine and hexamethylmelamine. In certain embodiments, the alkylating agent is cyclophosphamide.

Anti-metabolites are a group of molecules that impede DNA and RNA synthesis. Many of them have a similar structure to the building blocks of DNA and RNA. Anti-metabolites resemble either nucleobases or nucleosides, but have altered chemical groups. These drugs exert their effect by either blocking the enzymes required for DNA synthesis or becoming incorporated into DNA or RNA. Subtypes of the anti-metabolites are the anti-folates, fluoropyrimidines, deoxynucleoside analogues and thiopurines. The anti-folates include methotrexate and pemetrexed. The fluoropyrimidines include fluorouracil and capecitabine. The deoxynucleoside analogues include cytarabine, gemcitabine, decitabine, azacitidine, fludarabine, nelarabine, cladribine, clofarabine, and pentostatin. The thiopurines include thioguanine and mercaptopurine.

Anti-microtubule agents block cell division by preventing microtubule function. The vinca alkaloids prevent the formation of the microtubules, whereas the taxanes prevent the microtubule disassembly. Vinca alkaloids include vinorelbine, vindesine, and vinflunine. Taxanes include docetaxel (Taxotere) and paclitaxel (Taxol).

Topoisomerase inhibitors are drugs that affect the activity of two enzymes: topoisomerase I and topoisomerase II and include irinotecan, topotecan, camptothecin, etoposide, doxorubicin, mitoxantrone, teniposide, novobiocin, merbarone, and aclarubicin.

The cytotoxic antibiotics are a varied group of drugs that have various mechanisms of action. The common theme that they share in their chemotherapy indication is that they interrupt cell division. The most important subgroup is the anthracyclines (e.g., doxorubicin, daunorubicin, epirubicin, idarubicin pirarubicin, and aclarubicin) and the bleomycins; other prominent examples include mitomycin C, mitoxantrone, and actinomycin.

In some embodiments, e.g., prior to administration of immune effector cells, a lymphodepleting treatment may be applied, e.g., by administering cyclophosphamide and fludarabine. Such treatment may increase cell persistence and the incidence and duration of clinical responses.

“Immune checkpoint” refers to regulators of the immune system, and, in particular, co-stimulatory and inhibitory signals that regulate the amplitude and quality of T cell activity. In certain embodiments, the immune checkpoint is an inhibitory signal. In certain embodiments, the inhibitory signal is the interaction between PD-1 and PD-L¹ and/or PD-L².

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. “Programmed Death Ligand-1 (PD-L1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The term “PD-L²” as used herein includes human PD-L2 (hPD-L2), variants, isoforms, and species homologs of hPD-L², and analogs having at least one common epitope with hPD-L2. The ligands of PD-1 (PD-L1 and PD-L2) are expressed on the surface of antigen-presenting cells, such as dendritic cells or macrophages, and other immune cells. Binding of PD-1 to PD-L1 or PD-L2 results in downregulation of T cell activation. Cancer cells expressing PD-L1 and/or PD-L2 are able to switch off T cells expressing PD-1 which results in suppression of the anticancer immune response. The interaction between PD-1 and its ligands results in a decrease in tumor infiltrating lymphocytes, a decrease in T cell receptor mediated proliferation, and immune evasion by the cancerous cells. Immune suppression can be reversed by inhibiting the local interaction of PD-1 with PD-L1, and the effect is additive when the interaction of PD-1 with PD-L2 is blocked as well.

Many of the immune checkpoints are regulated by interactions between specific receptor and ligand pairs, such as those described above. Thus, immune checkpoint proteins mediate immune checkpoint signaling. For example, checkpoint proteins directly or indirectly regulate T cell activation, T cell proliferation and/or T cell function. Cancer cells often exploit these checkpoint pathways to protect themselves from being attacked by the immune system. Hence, the function of checkpoint proteins, which is modulated according to the present disclosure is typically the regulation of T cell activation, T cell proliferation and/or T cell function. Immune checkpoint proteins thus regulate and maintain self-tolerance and the duration and amplitude of physiological immune responses.

As used herein, the term “immune checkpoint modulator” or “checkpoint modulator” refers to a molecule or to a compound that modulates the function of one or more checkpoint proteins. Immune checkpoint modulators are typically able to modulate self-tolerance and/or the amplitude and/or the duration of the immune response. Preferably, the immune checkpoint modulator modulates the function of one or more human checkpoint proteins and is, thus, a “human checkpoint modulator”. Specifically, the human checkpoint modulator is an immune checkpoint inhibitor.

As used herein, “immune checkpoint inhibitor” or “checkpoint inhibitor” refers to a molecule that totally or partially reduces, inhibits, interferes with or negatively modulates one or more checkpoint proteins or that totally or partially reduces, inhibits, interferes with or negatively modulates expression of one or more checkpoint proteins. In certain embodiments, the immune checkpoint inhibitor binds to one or more molecules regulating checkpoint proteins.

In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule inhibitor that disrupts inhibitory signaling. In certain embodiments, the immune checkpoint inhibitor is a peptide-based inhibitor that disrupts inhibitory signaling.

In certain embodiments, the immune checkpoint inhibitor is an antibody, fragment thereof, or antibody mimic, that prevents the interaction between checkpoint blocker proteins.

In some embodiments, inhibiting or blocking of inhibitory immune checkpoint signaling, as described herein, results in preventing or reversing immune-suppression and establishment or enhancement of T cell immunity. In some embodiments, inhibition of immune checkpoint signaling, as described herein, reduces or inhibits dysfunction of the immune system. In some embodiments, inhibition of immune checkpoint signaling, as described herein, renders dysfunctional immune cells less dysfunctional. In some embodiments, inhibition of immune checkpoint signaling, as described herein, renders a dysfunctional T cell less dysfunctional.

In certain embodiments, the inhibitory immunoregulator (immune checkpoint blocker) is a component of the PD-1/PD-L1 or PD-1/PD-L2 signaling pathway.

In certain embodiments, the inhibitory immunoregulator (immune checkpoint blocker) is a PD-1 axis binding antagonist.

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In some embodiments, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody.

The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to one or more of its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In some embodiments, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In some embodiments, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

In some embodiments, a PD-1 axis binding antagonist includes a PD-1 binding antagonist, a PD-L1 binding antagonist and a PD-L2 binding antagonist. Alternative names for “PD-1” include CD279 and SLEB2. Alternative names for “PD-L1” include B7-H₁, B7-4, CD274, and B7-H. Alternative names for “PD-L2” include B7-DC. Btdc, and CD273. In some embodiments, PD-1, PD-L1, and PD-L2 are human PD-1. PD-L1 and PD-L2.

In some embodiments, the PD-1 binding antagonist is a molecule that inhibits the binding of PD-1 to its ligand binding partner(s). In a specific aspect, the PD-1 ligand binding partners are PD-L1 and/or PD-L2.

In some embodiments, the PD-L1 binding antagonist is a molecule that inhibits the binding of PD-L1 to its binding partner(s). In a specific aspect, PD-L1 binding partner(s) are PD-1 and/or B7-1.

In some embodiments, the PD-L2 binding antagonist is a molecule that inhibits the binding of PD-L2 to its binding partner(s). In a specific aspect, a PD-L2 binding partner is PD-1.

The antagonist may be an antibody, an antigen binding fragment thereof, an immunoadhesin, a fusion protein, or oligopeptide.

In some embodiments, the PD-1 binding antagonist is an anti-PD-1 antibody (e.g., a human antibody, a humanized antibody, or a chimeric antibody).

In some embodiments, the PD-L1 binding antagonist is an anti-PD-L1 antibody.

PD-1 axis binding antagonists such as anti-PD-1 antibodies and anti-PD-L1 antibodies may be administered in any manner and by any route known in the art. The mode and route of administration will depend on the type of PD-1 axis binding antagonist to be used.

PD-1 axis binding antagonists may be administered in the form of any suitable pharmaceutical composition as described herein.

PD-1 axis binding antagonists such as anti-PD-1 antibodies and anti-PD-L1 antibodies may be administered in the form of nucleic acid, such DNA or RNA, encoding a PD-1 axis binding antagonist such as anti-PD-1 antibody or anti-PD-L1 antibody. For example, antibodies can be delivered encoded in expressing nucleic acids, as described herein. Nucleic acid molecules can be delivered as such, e.g., in the form of a plasmid or mRNA molecule, or complexed with a delivery vehicle, e.g., a liposome, lipoplex or any other nucleic-acid particle such as nucleic-acid lipid particle. PD-1 axis binding antagonists such as anti-PD-1 antibodies and anti-PD-L1 antibodies may also be administered via an oncolytic virus comprising an expression cassette encoding the PD-1 axis binding antagonist.

### Compositions Comprising Nucleic Acid (Such as DNA or RNA) Particles

In some embodiments, the compositions of the present disclosure comprising particles (such as LNPs) may comprise a plurality of nucleic acid (such as DNA or RNA) particles (in particular a plurality of RNA LNPs).

The term “plurality of nucleic acid particles” or “plurality of nucleic acid-lipid particles” (such as “plurality of RNA particles” or “plurality of RNA-lipid particles”) refers to a population of a certain number of particles. In certain embodiments, the term refers to a population of more than 10, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², or 10²³ or more particles.

It will be apparent to those of skill in the art that the plurality of particles can include any fraction of the foregoing ranges or any range therein.

In some embodiments, the composition of the present disclosure is a liquid or a solid. Non-limiting examples of a solid include a frozen form, a lyophilized form or a spray-dried form. In certain embodiments, the composition is a liquid.

A composition comprising one or more nucleic acids (such as DNA or RNAs) described herein, e.g., in the form of nucleic acid (such as DNA or RNA) particles, may comprise salts, buffers, or other components as further described below.

In some embodiments, a salt for use in the compositions described herein comprises sodium chloride. Without wishing to be bound by theory, sodium chloride functions as an ionic osmolality agent for preconditioning RNA prior to mixing with lipids. In some embodiments, the compositions described herein may comprise alternative organic or inorganic salts. Alternative salts include, without limitation, potassium chloride, dipotassium phosphate, monopotassium phosphate, potassium acetate, potassium bicarbonate, potassium sulfate, disodium phosphate, monosodium phosphate, sodium acetate, sodium bicarbonate, sodium sulfate, lithium chloride, magnesium chloride, magnesium phosphate, calcium chloride, and sodium salts of ethylenediaminetetraacetic acid (EDTA).

According to the present disclosure, the compositions described herein may comprise salts such as organic or inorganic salts, including, but not limited to, sodium chloride, potassium chloride, potassium acetate, potassium acetate, sodium acetate, lithium chloride, magnesium chloride, calcium chloride, and sodium salts of amino acids.

Generally, compositions for storing RNA particles such as for freezing RNA particles comprise low sodium chloride concentrations, or comprises a low ionic strength. In some embodiments, the sodium chloride is at a concentration from 0 mM to about 50 mM, from 0 mM to about 40 mM, or from about 10 mM to about 50 mM.

According to the present disclosure, the nucleic acid (such as DNA or RNA) compositions (such as the nucleic acid (such as DNA or RNA) particle compositions) described herein have a pH suitable for the stability of the nucleic acid (such as RNA) particles and, in particular, for the stability of the nucleic acid (such as RNA). Without wishing to be bound by theory, the use of a buffer system maintains the pH of the particle compositions described herein during manufacturing, storage and use of the compositions. In some embodiments of the present disclosure, the buffer system may comprise a solvent (in particular, water, such as deionized water, in particular water for injection) and a buffering substance.

The buffering substance may be selected from 2-[4-(2-hydroxyethyl) piperazin-1-yl]ethanesulfonic acid (HEPES), 2-amino-2-(hydroxymethyl)-propane-1,3-diol (Tris), acetate, and histidine. In some embodiments, the buffering substance is HEPES.

Compositions (in particular, RNA compositions/formulations) described herein may also comprise one or more stabilizers to avoid substantial loss of the product quality and, in particular, substantial loss of RNA activity during storage, freezing, lyophilization and/or spray-drying, for example to reduce or prevent aggregation, particle collapse, RNA degradation and/or other types of damage.

In some embodiments, the one or more stabilizers may comprise a cryoprotectant, a surfactant, an amino acid, and/or lyoprotectant.

In some embodiments, the one or more stabilizers comprise a lyoprotectant.

In some embodiments, the one or more stabilizers may comprise a cryoprotectant.

In some embodiments, the cryoprotectant is a carbohydrate. The term “carbohydrate”, as used herein, refers to and encompasses monosaccharides, disaccharides, trisaccharides, oligosaccharides and polysaccharides.

In some embodiments, the cryoprotectant is a monosaccharide. The term “monosaccharide”, as used herein refers to a single carbohydrate unit (e.g., a simple sugar) that cannot be hydrolyzed to simpler carbohydrate units. Exemplary monosaccharide cryoprotectants include glucose, fructose, galactose, xylose, ribose and the like.

In some embodiments, the cryoprotectant is a disaccharide. The term “disaccharide”, as used herein refers to a compound or a chemical moiety formed by 2 monosaccharide units that are bonded together through a glycosidic linkage, for example through 1-4 linkages or 1-6 linkages. A disaccharide may be hydrolyzed into two monosaccharides. Exemplary disaccharide cryoprotectants include sucrose, trehalose, lactose, maltose and the like. In some embodiments, the cryoprotectant is sucrose.

The term “trisaccharide” means three sugars linked together to form one molecule. Examples of a trisaccharides include raffinose and melezitose.

In some embodiments, the cryoprotectant is an oligosaccharide. The term “oligosaccharide”, as used herein refers to a compound or a chemical moiety formed by 3 to about 15, such as 3 to about 10 monosaccharide units that are bonded together through glycosidic linkages, for example through 1-4 linkages or 1-6 linkages, to form a linear, branched or cyclic structure. Exemplary oligosaccharide cryoprotectants include cyclodextrins, raffinose, melezitose, maltotriose, stachyose, acarbose, and the like. An oligosaccharide can be oxidized or reduced.

In an embodiment, the cryoprotectant is a cyclic oligosaccharide. The term “cyclic oligosaccharide”, as used herein refers to a compound or a chemical moiety formed by 3 to about 15, such as 6, 7, 8, 9, or 10 monosaccharide units that are bonded together through glycosidic linkages, for example through 1-4 linkages or 1-6 linkages, to form a cyclic structure. Exemplary cyclic oligosaccharide cryoprotectants include cyclic oligosaccharides that are discrete compounds, such as a cyclodextrin, β cyclodextrin, or γ cyclodextrin.

Other exemplary cyclic oligosaccharide cryoprotectants include compounds which include a cyclodextrin moiety in a larger molecular structure, such as a polymer that contains a cyclic oligosaccharide moiety. A cyclic oligosaccharide can be oxidized or reduced, for example, oxidized to dicarbonyl forms. The term “cyclodextrin moiety”, as used herein refers to cyclodextrin (e.g., an α, β, or γ cyclodextrin) radical that is incorporated into, or a part of, a larger molecular structure, such as a polymer. A cyclodextrin moiety can be bonded to one or more other moieties directly, or through an optional linker. A cyclodextrin moiety can be oxidized or reduced, for example, oxidized to dicarbonyl forms.

Carbohydrate cryoprotectants, e.g., cyclic oligosaccharide cryoprotectants, can be derivatized carbohydrates. For example, in an embodiment, the cryoprotectant is a derivatized cyclic oligosaccharide, e.g., a derivatized cyclodextrin, e.g., 2-hydroxypropyl-β-cyclodextrin, e.g., partially etherified cyclodextrins (e.g., partially etherified β cyclodextrins).

An exemplary cryoprotectant is a polysaccharide. The term “polysaccharide”, as used herein refers to a compound or a chemical moiety formed by at least 16 monosaccharide units that are bonded together through glycosidic linkages, for example through 1-4 linkages or 1-6 linkages, to form a linear, branched or cyclic structure, and includes polymers that comprise polysaccharides as part of their backbone structure. In backbones, the polysaccharide can be linear or cyclic. Exemplary polysaccharide cryoprotectants include glycogen, amylase, cellulose, dextran, maltodextrin and the like.

In some embodiments, nucleic acid (such as DNA or RNA) compositions (such as the nucleic acid (such as DNA or RNA) particle compositions) may include sucrose. Without wishing to be bound by theory, sucrose functions to promote cryoprotection of the compositions, thereby preventing nucleic acid (such as DNA or RNA, especially mRNA) particle aggregation and maintaining chemical and physical stability of the composition. In some embodiments, nucleic acid (such as DNA or RNA) compositions (such as the nucleic acid (such as DNA or RNA) particle compositions) may include alternative cryoprotectants to sucrose. Alternative stabilizers include, without limitation, trehalose and glucose. In a specific embodiment, an alternative stabilizer to sucrose is trehalose or a mixture of sucrose and trehalose.

A preferred cryoprotectant is selected from the group consisting of sucrose, trehalose, glucose, and a combination thereof, such as a combination of sucrose and trehalose. In certain embodiments, the cryoprotectant is sucrose.

In some embodiments, the one or more stabilizers comprise an amino acid or a surfactant (e.g. poloxamer).

In some embodiments, the one or more stabilizers comprise a cryoprotectant and a surfactant. In some embodiments, the one or more stabilizers comprise a cryoprotectant and a polyoxamer.

Some embodiments of the present disclosure contemplate the use of a chelating agent in a nucleic acid (such as DNA or RNA) composition described herein. Chelating agents refer to chemical compounds that are capable of forming at least two coordinate covalent bonds with a metal ion, thereby generating a stable, water-soluble complex. Without wishing to be bound by theory, chelating agents reduce the concentration of free divalent ions, which may otherwise induce accelerated RNA degradation in the present disclosure. Examples of suitable chelating agents include, without limitation, ethylenediaminetetraacetic acid (EDTA), a salt of EDTA, desferrioxamine B, deferoxamine, dithiocarb sodium, penicillamine, pentetate calcium, a sodium salt of pentetic acid, succimer, trientine, nitrilotriacetic acid, trans-diaminocyclohexanetetraacetic acid (DCTA), diethylenetriaminepentaacetic acid (DTPA), and bis(aminoethyl)glycolether-N,N,N′,N′-tetraacetic acid. In some embodiments, the chelating agent is EDTA or a salt of EDTA. In some embodiments, the chelating agent is EDTA disodium dihydrate. In some embodiments, the EDTA is at a concentration from about 0.05 mM to about 5 mM, from about 0.1 mM to about 2.5 mM or from about 0.25 mM to about 1 mM.

In an alternative embodiment, the nucleic acid (such as DNA or RNA) compositions (such as the nucleic acid (such as DNA or RNA) particle compositions) described herein do not comprise a chelating agent.

According to the present disclosure, the nucleic acid (such as DNA or RNA) compositions (such as the nucleic acid (such as DNA or RNA) particle compositions, in particular RNA LNP compositions) described herein have a pH suitable for the stability of the nucleic acid (such as DNA or RNA) particles and, in particular, for the stability of the nucleic acid (such as DNA or RNA). In some embodiments, the RNA particle compositions described herein have a pH from about 4.0 to about 8.0, or about 5.0 to about 7.5. Without wishing to be bound by theory, the use of buffer maintains the pH of the composition during manufacturing, storage and use of the composition. In certain embodiments of the present disclosure, the buffer may be 2-(bis(2-hydroxyethyl)amino)acetic acid (Bicine), 2-amino-2-(hydroxymethyl) propane-1,3-diol (Tris), or N-(2-Hydroxy-1,1-bis(hydroxymethyl)ethyl) glycine (Tricine). Other suitable buffering systems may be acetic acid alone or in a salt, or amino acids and amino acid derivatives.

In some embodiments, the composition described herein comprises water as the main component and/or the total amount of solvent(s) other than water contained in the composition is less than about 1.0% (v/v). For example, the amount of water contained in the composition may be at least 50% (w/w), such as at least at least 55% (w/w), at least 60% (w/w), at least 65% (w/w), at least 70% (w/w), at least 75% (w/w), at least 80% (w/w), at least 85% (w/w), at least 90% (w/w), or at least 95% (w/w). In particular, if the composition comprises a cryoprotectant, the amount of water contained in the composition may be at least 50% (w/w), such as at least at least 55% (w/w), at least 60% (w/w), at least 65% (w/w), at least 70% (w/w), at least 75% (w/w), at least 80% (w/w), at least 85% (w/w), or at least 90% (w/w). If the composition is substantially free of a cryoprotectant, the amount of water contained in the composition may be at least 95% (w/w). Additionally or alternatively, the total amount of solvent(s) other than water contained in the composition may be less than about 1.0% (v/v), such as less than about 0.9% (v/v), less than about 0.8% (v/v), less than about 0.7% (v/v), less than about 0.6% (v/v), less than about 0.5% (v/v), less than about 0.4% (v/v), less than about 0.3% (v/v), less than about 0.2% (v/v), less than about 0.1% (v/v), less than about 0.05% (v/v), or less than about 0.01% (v/v). In this respect, a cryoprotectant which is liquid under normal conditions will not be considered as a solvent other than water but as cryoprotectant. In other words, the above optional limitation that the total amount of solvent(s) other than water contained in the composition may be less than about 1.0% (v/v) does not apply to cryoprotectants which are liquids under normal conditions.

### Pharmaceutical Compositions

The compositions comprising nucleic acid (such as DNA or RNA), such as nucleic acid (such as DNA or RNA) particles, in particular RNA LNPs) described herein are useful as or for preparing pharmaceutical compositions or medicaments for therapeutic or prophylactic treatments.

In one aspect, nucleic acid (such as DNA or RNA) particles (in particular RNA LNPs) described herein are present in a pharmaceutical composition. In another aspect, a composition described herein is a pharmaceutical composition.

The particles of the present disclosure may be administered in the form of any suitable pharmaceutical composition.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation. In the context of the present disclosure, the pharmaceutical composition comprises nucleic acid (such as DNA or RNA) particles as described herein.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cyctokines, such as monokines, lymphokines, interleukins, chemokines. The chemokines may be IL-1, IL-2, IL-3, IL-4, IL-5, IL-6, IL-7, IL-8, IL-9, IL-10, IL-12, INFa, INF-γ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys, as well as hydrophobic or lipophilic components, such as saponins, trehalose-6,6-dibehenate (TDB), monophosphoryl lipid-A (MPL), monomycoloyl glycerol (MMG), or glucopyranosyl lipid adjuvant (GLA).

The pharmaceutical compositions of the present disclosure may be in a storable form (e.g., in a frozen or lyophilized/freeze-dried form) or in a “ready-to-use form” (i.e., in a form which can be immediately administered to a subject, e.g., without any processing such as diluting). Thus, prior to administration of a storable form of a pharmaceutical composition, this storable form has to be processed or transferred into a ready-to-use or administrable form. E.g., a frozen pharmaceutical composition has to be thawed, or a freeze-dried pharmaceutical composition has to be reconstituted, e.g. by using a suitable solvent (e.g., deionized water, such as water for injection) or liquid (e.g., an aqueous solution).

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the particles or compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the particles or compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In some embodiments, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carriers include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In some embodiments, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

### Routes of Administration of Pharmaceutical Compositions

In some embodiments, pharmaceutical compositions described herein may be administered intravenously, intraarterially, subcutaneously, intradermally, dermally, intramuscularly, intratumorally, intranasally, rectally or orally. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In certain embodiments, the pharmaceutical compositions is formulated for systemic administration. In other certain embodiments, the systemic administration is by intravenous administration.

### Use of Pharmaceutical Compositions

Nucleic acid (such as DNA or RNA) compositions (such as the nucleic acid (such as DNA or RNA) particles) described herein may be used in the therapeutic or prophylactic treatment of various diseases, in particular diseases in which provision of a peptide or protein to a subject results in a therapeutic or prophylactic effect. For example, provision of an antigen or epitope which is derived from a virus may be useful in the treatment of a viral disease caused by said virus. Provision of a tumor antigen or epitope may be useful in the treatment of a cancer disease wherein cancer cells express said tumor antigen. Provision of a functional protein or enzyme may be useful in the treatment of genetic disorder characterized by a dysfunctional protein, for example in lysosomal storage diseases (e.g. Mucopolysaccharidoses) or factor deficiencies. Provision of a cytokine or a cytokine-fusion may be useful to modulate tumor microenvironment.

The term “disease” (also referred to as “disorder” herein) refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

The term “disease involving an antigen” refers to any disease which implicates an antigen, e.g. a disease which is characterized by the presence of an antigen. The disease involving an antigen can be an infectious disease, or a cancer disease or simply cancer. The antigen may be a disease-associated antigen, such as a tumor-associated antigen, a viral antigen, or a bacterial antigen. In some embodiments, a disease involving an antigen is a disease involving cells expressing an antigen, and preferably presenting the antigen on the cell surface, e.g., in the context of MHC.

The term “infectious disease” refers to any disease which can be transmitted from individual to individual or from organism to organism, and is caused by a microbial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), HIV/acquired immune deficiency syndrome (AIDS), diphtheria, hepatitis B, hepatitis C. cholera, severe acute respiratory syndrome (SARS), the bird flu, and influenza.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g., mouse, rat, rabbit, dog, cat, cattle, swine, sheep, horse or primate), or any other non-mammal-animal, including birds (chicken), fish or any other animal species that can be afflicted with or is susceptible to a disease or disorder (e.g., cancer, infectious diseases) but may or may not have the disease or disorder, or may have a need for prophylactic intervention such as vaccination, or may have a need for interventions such as by protein replacement. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

Nucleic acid, in particular RNA, may be administered to a subject for delivering the nucleic acid to cells of the subject.

Nucleic acid, in particular RNA, may be administered to a subject for delivering a therapeutic or prophylactic peptide or polypeptide (e.g., a pharmaceutically active peptide or polypeptide) to the subject, wherein the nucleic acid encodes a therapeutic or prophylactic peptide or polypeptide.

Nucleic acid, in particular RNA, may be administered to a subject for treating or preventing a disease in a subject, wherein delivering the nucleic acid to cells of the subject is beneficial in treating or preventing the disease.

Nucleic acid, in particular RNA, may be administered to a subject for treating or preventing a disease in a subject, wherein the nucleic acid encodes a therapeutic or prophylactic peptide or polypeptide and wherein delivering the therapeutic or prophylactic peptide or polypeptide to the subject is beneficial in treating or preventing the disease.

In some embodiments, the nucleic acid is present in a composition as described herein.

In some embodiments, the nucleic acid is administered in a pharmaceutically effective amount.

In some embodiments, the subject is a mammal. In some embodiments, the mammal is a human.

In some embodiments of the disclosure, the aim is to induce an immune response by providing a vaccine.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, nucleic acids (in particular RNAs) described herein are applicable for inducing or enhancing an immune response. Nucleic acids described herein are thus useful in a prophylactic and/or therapeutic treatment of a disease involving an antigen or epitope.

In some embodiments of the disclosure, the aim is to provide an immune response against diseased cells expressing an antigen such as cancer cells expressing a tumor antigen, and to treat a disease such as a cancer disease involving cells expressing an antigen such as a tumor antigen.

In some embodiments of the disclosure, the aim is to treat cancer by vaccination.

In some embodiments of the disclosure, the aim is to provide an immune response against cancer cells expressing a tumor antigen and to treat a cancer disease involving cells expressing a tumor antigen.

In some embodiments of the disclosure, the aim is to provide protection against an infectious disease by vaccination.

In some embodiments of the disclosure, the aim is to provide secreted therapeutic proteins, such as antibodies, bispecific antibodies, cytokines, cytokine fusion proteins, enzymes, to a subject, in particular a subject in need thereof.

In some embodiments of the disclosure, the aim is to provide a protein replacement therapy, such as production of erythropoietin, Factor VII, Von Willebrand factor, β-galactosidase, Alpha-N-acetylglucosaminidase, to a subject, in particular a subject in need thereof.

In some embodiments of the disclosure, the aim is to modulate/reprogram immune cells in the blood.

In some embodiments of the disclosure, the aim is to provide one or more cytokines or cytokine fusions which modulate tumor microenvironment to a subject, in particular a subject in need thereof.

In some embodiments of the disclosure, the aim is to provide one or more cytokines or cytokine fusions which have antitumoral activity to a subject, in particular a subject in need thereof.

A person skilled in the art will know that one of the principles of immunotherapy and vaccination is based on the fact that an immunoprotective reaction to a disease is produced by immunizing a subject with an antigen or an epitope, which is immunologically relevant with respect to the disease to be treated. Accordingly, pharmaceutical compositions described herein are applicable for inducing or enhancing an immune response. Pharmaceutical compositions described herein are thus useful in a prophylactic and/or therapeutic treatment of a disease involving an antigen or epitope.

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Itemized Embodiments

1. A composition comprising lipid nanoparticles (LNPs), wherein the LNPs comprise:
   (i) RNA;
   (ii) a cationic or cationically ionizable lipid; and
   (iii) a conjugate of (a) a polyoxazoline (POX) and/or polyoxazine (POZ) polymer and (b) one or more hydrophobic chains.
2. The composition of item 1, wherein the total number of POX and/or POZ repeating units in the polymer is between 2 and 200.
3. The composition of item 1 or 2, wherein the POX and/or POZ polymer comprises the following general formula (I):

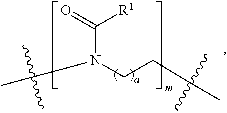

wherein a is an integer between 1 and 2; R¹ is alkyl, in particular C₁₋₃ alkyl, such as methyl, ethyl, iso-propyl, or n-propyl, and is independently selected for each repeating unit; and m is 2 to 200.
4. The composition of any one of items 1 to 3, wherein the conjugate has the following general formula (II) or (II′):

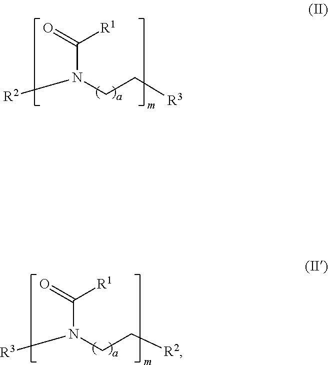

wherein:
a is an integer between 1 and 2;
R¹ is alkyl, in particular C₁₋₃ alkyl, such as methyl, ethyl, iso-propyl, or n-propyl, and is independently selected for each repeating unit;
m is 2 to 200;
R² is R⁴ or -L¹(R⁴)ₚ, wherein each R⁴ is independently a hydrocarbyl group; L¹ is a linker; and
p is 1 or 2; and
R³ is selected from the group consisting of H, C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.
4a. The composition of item 4, wherein the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor.
5. The composition of item 3, 4 or 4a, wherein R¹ is methyl or ethyl, preferably methyl.
6. The composition of any one of items 3 to 5, wherein m is 2 to 180, such as 4 to 160, 6 to 140, 8 to 120 or 10 to 100.
7. The composition of any one of items 4 to 6, wherein L¹ comprises at least one functional moiety, such as an alkylene moiety substituted with at least one monovalent functional moiety and/or linked, at the end by which the alkylene group is attached to R⁴, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.
7a. The composition of any one of items 4 to 7, wherein the conjugate comprises one of the following structures and optionally has the general formula (II):
   (hydrophobic chain)₁₋₂-(alkylene moiety substituted with at least one monovalent functional moiety)-(POX and/or POZ polymer)
   [(hydrophobic chain)-(divalent functional moiety)]₁₋₂-(alkylene moiety)-(POX and/or POZ polymer).
7b. The composition of any one of items 4 to 7a, wherein the conjugate has one of the following formulas and optionally has the general formula (II):
   (hydrophobic chain)₁₋₂-(alkylene moiety substituted with at least one monovalent functional moiety)-(POX and/or POZ polymer)-R³
   [(hydrophobic chain)-(divalent functional moiety)]₁₋₂-(alkylene moiety)-(POX and/or POZ polymer)-R³.
7c. The composition of any one of items 7 to 7b, wherein the alkylene moiety substituted with at least one monovalent functional moiety is substituted with 1, 2, 3, 4, 5, or 6 independently selected monovalent functional moieties.
7d. The composition of any one of items 7 to 7c, wherein the alkylene moiety is C₁₋₆-alkylene, such as C₁₋₃-alkylene, e.g., methylene, ethylene, or trimethylene.
7e. The composition of any one of items 4 to 7, wherein the conjugate has the general formula (II′) and comprises one of the following structures:
   (hydrophobic chain)-(divalent functional moiety)-(POX and/or POZ polymer)
   [(hydrophobic chain)-(divalent functional moiety)]₁₋₂-(alkylene moiety)-(divalent functional moiety)-(POX and/or POZ polymer)
   (hydrophobic chain)-(divalent functional moiety)-(cycloalkylene moiety)-(divalent functional moiety)-(POX and/or POZ polymer)
   (hydrophobic chain)-(divalent functional moiety)-(cycloalkenylene moiety)-(divalent functional moiety)-(POX and/or POZ polymer)
   (hydrophobic chain)-(divalent functional moiety)-(alkylene moiety)-(POX and/or POZ polymer)
   [(hydrophobic chain)₂-(trivalent functional moiety)]-(alkylene moiety)-(divalent functional moiety)-(POX and/or POZ polymer)
7f. The composition of any one of items 4 to 7 and 7e, wherein the conjugate has the general formula (II′) and has one of the following formulas:
   (hydrophobic chain)-(divalent functional moiety)-(POX and/or POZ polymer)-R³
   [(hydrophobic chain)-(divalent functional moiety)]₁₋₂-(alkylene moiety)-(divalent functional moiety)-(POX and/or POZ polymer)-R³
   (hydrophobic chain)-(divalent functional moiety)-(cycloalkylene moiety)-(divalent functional moiety)-(POX and/or POZ polymer)-R³
   (hydrophobic chain)-(divalent functional moiety)-(cycloalkenylene moiety)-(divalent functional moiety)-(POX and/or POZ polymer)-R³
   (hydrophobic chain)-(divalent functional moiety)-(alkylene moiety)-(POX and/or POZ polymer)-R³
   [(hydrophobic chain)₂-(trivalent functional moiety)]-(alkylene moiety)-(divalent functional moiety)-(POX and/or POZ polymer)-R³
7g. The composition of item 7e or 7f, wherein the alkylene moiety is C₁₋₆-alkylene, such as C₁₋₃-alkylene, e.g., methylene, ethylene, or trimethylene.
7 h. The composition of any one of items 7e to 7g, wherein the cycloalkylene moiety is C₃₋₈-cycloalkylene, such as C₃₋₆-cycloalkylene, e.g., cyclopropylene, cyclobutylene, cyclopentylene, cyclohexylene; and/or the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein each of the cycloalkylene and cycloalkenylene moieties is optionally substituted with one or more (e.g., 1, 2, 3, or 4) substituents (e.g., independently selected from the group consisting of —OH, ═O, —SH, halogen, —CN, —N₃, and C₁₋₃-alkyl).
7i. The composition of any one of items 4 to 7, wherein the conjugate comprises the following structure and optionally has the general formula (II):
[(hydrophobic chain)₂-(trivalent functional moiety)]-(alkylene moiety)-(POX and/or POZ polymer).
7j. The composition of any one of items 4 to 7 and 7j, wherein the conjugate has the following formula and optionally has the general formula (II):
   [(hydrophobic chain)₂-(trivalent functional moiety)]-(alkylene moiety)-(POX and/or POZ polymer)-R³.
8. The composition of any one of items 4 to 7d, wherein L¹ is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR²⁵)₃₋ᵣ](C₁₋₆-alkylene)-, [*—C(OR²⁵)₂O]ₚ(C₁₋₆-alkylene)-, [*—C(R²⁵)(═N—N(R²⁶)C(O)—)]ₚ(C₁₋₆-alkylene)-, [*—C(O)(N(R²⁶)—N═)C(R²⁵)—]ₚ(C₁₋₆-alkylene)-, [*═C(═N—N(R²⁶)C(O)(R²⁵))]ₚ(C₁₋₆-alkylene)-, [*—N(R²⁶)N(R²⁶)]ₚ(C₁₋₆-alkylene)-, [*═C(═N(OH))]ₚ(C₁₋₆-alkylene)-, and [*—OC(R²⁵)(R²⁶)O]ₚ(C₁₋₆-alkylene)-, wherein * represents the attachment point to R⁴; p is 1 or 2; C₁₋₆-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2.
8a. The composition of any one of items 4 to 7d and 8, wherein L¹ is selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, [*—SS]ₚ(C₁₋₃-alkylene)-, [*—S(O)₂]ₚ(C₁₋₃-alkylene)-, [(*—O)ᵣC(OR²⁵)₃ . . . ]—(C₁₋₃-alkylene)-, [*—C(OR²⁵)₂O]ₚ(C₁₋₃-alkylene)-, [*—C(R²⁵)(═N—N(R²⁶)C(O)—)]ₚ(C₁₋₃-alkylene)-, [*—C(O)(N(R²⁶)—N═)C(R²⁵)—]ₚ(C₁₋₃-alkylene)-, [*═C(═N—N(R²⁶)C(O)(R²⁵))]ₚ(C₁₋₃-alkylene)-, [*—N(R²⁶)N(R²⁶)]ₚ(C₁₋₃-alkylene)-, [*═C(═N(OH))]ₚ(C₁₋₃-alkylene)-, and [*—OC(R²⁵)(R²⁶)O]ₚ—(C₁₋₃-alkylene)-, wherein * represents the attachment point to R⁴; p is 1 or 2; C₁₋₃-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2.
8b. The composition of item 8 or 8a, wherein R²⁵ is selected from the group consisting of C₁₋₃ alkyl, phenyl, and phenyl(C₁₋₃ alkyl); and/or R²⁶ is selected from the group consisting of H, C₁₋₃ alkyl, phenyl, and phenyl(C₁₋₃ alkyl).
8c. The composition of any one of items 4 to 7d and 8, wherein L¹ is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, preferably from the group consisting of [*—NHC(O)]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, more preferably from the group consisting of [*—NHC(O)]ₚ(C₁₋₆-alkylene)- and [*—C(O)O]ₚ(C₁-6-alkylene)-, wherein * represents the attachment point to R⁴; p is 1 or 2; and C₁₋₆-alkylene is either bivalent (if p is 1) or trivalent (if p is 2).
8d. The composition of item 8c, wherein L¹ is selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, and [*—S(O)₂]ₚ(C₁₋₃-alkylene)-, preferably from the group consisting of [*—NHC(O)]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, and [*—S(O)₂]ₚ(C₁₋₃-alkylene)-, more preferably from the group consisting of [*—NHC(O)]ₚ(C₁₋₃-alkylene)- and [*—C(O)O]ₚ(C₁₋₃-alkylene)-, wherein * represents the attachment point to R⁴; p is 1 or 2; and C₁₋₃-alkylene is either bivalent (if p is 1) or trivalent (if p is 2).
9. The composition of any one of items 4 to 7d and 8 to 8d, wherein L¹ is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R⁴.
10. The composition of any one of items 4 to 7d, 8 to 8d, and 9, wherein R² is selected from the group consisting of R⁴, -L¹R⁴, —(CH₂)—CH(OC(O)R⁴)(CH₂OC(O)R⁴), —(CH₂)—CH(SR⁴)₂, and (CH₂)—CH(SR⁴)—CH₂(SR⁴); and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L¹ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴.
10a. The composition of any one of items 4 to 7d, and 8 to 10, wherein R² is-L¹(R⁴)ₚ.
11. The composition of any one of items 4 to 7, 7i, and 7j, wherein L¹ is selected from the group consisting of [*-]₂NC(O)(C₁₋₆-alkylene)-, [*—C(O)O]ₚ(C₁₋₆-alkylene)-OP(O)₂O(C₁₋₆-alkylene) NHC(O)—(C₁₋₆-alkylene)-, [*—OC(O)]ₚ(C₁₋₆-alkylene)-OP(O)₂O(C₁₋₆-alkylene) NHC(O)—(C₁₋₆-alkylene)-, and [*—C(O)]ₚ(C₁₋₆-alkylene)-C(O)—, wherein * represents the attachment point to R⁴; p is 1 or 2; and C₁₋₆-alkylene is either bivalent (if p is 1) or trivalent (if p is 2).
12. The composition of any one of items 4 to 7, 7i, 7j, and 11, wherein L¹ is selected from the group consisting of [*-]₂NC(O)(C₁₋₂-alkylene)-, [*—OC(O)]₂ (C₂₋₄-alkylene)-OP(O)₂O(C₂₋₃-alkylene) NHC(O)—(C₁₋₂-alkylene)-, and *—C(O)(C₂₋₃-alkylene)-C(O)—, wherein * represents the attachment point to R⁴.
13. The composition of any one of items 4 to 7, 7i, 7j, 11, and 12, wherein R² is selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—.
13a. The composition of any one of items 7i, 7j, and 11 to 13, wherein R² is-L¹(R⁴)ₚ.
14. The composition of any one of items 4 to 7 and 7e to 7h, wherein the conjugate has the general formula (II′) and L¹ is selected from the group consisting of [*—Z]ₚ(C₁₋₆-alkylene)-Z—, *—Z—(C₃₋₈-cycloalkylene)-Z—, *—Z—(C₃₋₈-cycloalkenylene)-Z—, (*═N)(C₁₋₆-alkylene)-Z—, *—Z—(C₁₋₆-alkylene)-, and *—Z—, wherein * represents the attachment point to R⁴; p is 1 or 2; C₁₋₆-alkylene is either bivalent (if p is 1) or trivalent (if p is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(C₁₋₃-alkylene) NH—, —NH(C₁₋₃-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—.
14a. The composition of any one of items 4 to 7, 7e to 7h, and 14, wherein the conjugate has the general formula (II′) and L¹ is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z—(C₁₋₃-alkylene)-, and *—Z—, wherein * represents the attachment point to R⁴; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(C₁₋₂-alkylene) NH—, —NH(C₁₋₂-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—.
14b. The composition of any one of items 4 to 7, 7e to 7 h, 14, and 14a, wherein the conjugate has the general formula (II′) and L¹ is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment point to R⁴; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—.
14c. The composition of any one of items 4 to 7, 7e to 7 h, 14, 14a, and 14b, wherein the conjugate has the general formula (II′) and L¹ is selected from the group consisting of (*—C(O)O)(CH(OC(O)—*))(CH₂)—Z—, (*═N)(C₁₋₃-alkylene)-NHC(O)—, (*—Z) (C₁₋₃-alkylene)-Z—, *—Z—(C₃₋₆-cycloalkenylene)-Z—, and *—Z—, wherein * represents the attachment point to R⁴; the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
14d. The composition of any one of items 4 to 7, 7e to 7h, and 14 to 14c, wherein the conjugate has the general formula (II′) and R² is selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
14e. The composition of any one of items 7e to 7 h and 14 to 14d, wherein R² is-L¹(R⁴)ₚ.
15. The composition of any one of items 4 to 7, wherein the conjugate has the general formula (II′) and L¹ is [*—C(O)]ₚ(C₁₋₆-alkylene)-C(O)NH— or [*-]₂N—, wherein * represents the attachment point to R⁴; p is 1 or 2; and C₁₋₆-alkylene is either bivalent (if p is 1) or trivalent (if p is 2).
16. The composition of any one of items 4 to 7 and 15, wherein the conjugate has the general formula (II′) and L¹ is *—C(O)(C₂₋₃-alkylene)-C(O)NH— or [*-]₂N—, wherein * represents the attachment point to R⁴.
17. The composition of any one of items 4 to 7, 15, and 16, wherein the conjugate has the general formula (II′) and R² is R⁴—C(O)(CH₂)₂C(O)NH— or R⁴N—.
17a. The composition of any one of items 15 to 17, wherein R² is-L¹(R⁴)ₚ.
18. The composition of any one of items 4 to 17a, wherein each R⁴ is independently a non-cyclic, preferably straight hydrocarbyl group.
19. The composition of any one of items 4 to 18, wherein each R⁴ is independently a hydrocarbyl group having at least 8 carbon atoms, such as at least 10 carbon atoms.
19a. The composition of any one of items 4 to 19, wherein each R⁴ is independently a hydrocarbyl group having at most 16 carbon atoms, such as at most 14 carbon atoms or at most 12 carbon atoms.
19b. The composition of any one of items 4 to 19, wherein the conjugate has the general formula (II′) and each R⁴ is independently a hydrocarbyl group having 10 to 18 carbon atoms, such as a straight alkyl group having 10 to 18 carbon atoms or a straight alkenyl group having 10 to 18 carbon atoms.
20. The composition of any one of items 4 to 17a and 19, wherein one R⁴ is a tocopherol moiety.
21. The composition of any one of items 4 to 20, wherein R³ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.
22. The composition of any one of items 4 to 21, wherein R³ is selected from the group consisting of H, C₁₋₃ alkyl, —OR²⁰, —N₃, C₂₋₆ alkynyl, —OC(O)R²¹, —C(O)R²¹, —NR²²R²³, —COOH, Z—C(O)NR²²R²³, —NR²²C(O)R²¹, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, —C(O)NR²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group.
23. The composition of any one of items 4 to 22, wherein R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
24. The composition of any one of items 1 to 23, wherein the conjugate has the following general formula (III) or (III′):

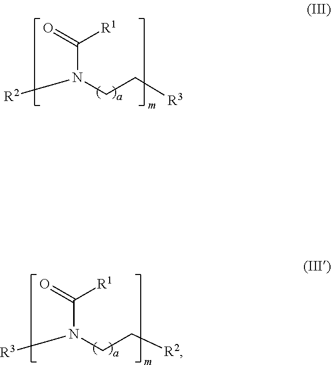

wherein:
a is an integer between 1 and 2;
R¹ is methyl or ethyl and is independently selected for each repeating unit;
m is 10 to 100;
R², for formula (III), is selected from the group consisting of -L¹R⁴, —(CH₂)—CH(OC(O)R⁴)(CH₂OC(O)R⁴), —(CH₂)—CH(SR⁴)₂, and —(CH₂)—CH(SR⁴)—CH₂(SR⁴), wherein each R⁴ is independently a straight hydrocarbyl group having at least 10 carbon atoms; and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L¹ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴; or R², for formula (III′), is selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
24a. The composition of item 24, wherein the conjugate has formula (III) and R² is selected from the group consisting of R⁴—NHC(O)—(CH₂)—, R⁴—NHC(O)—(CH₂)₂—, —(CH₂)—CH(OC(O)R⁴)(CH₂OC(O)R⁴), —(CH₂)—CH(SR⁴)—CH₂(SR⁴), R⁴S—(CH₂)₃—, R⁴S(O)₂—(CH₂)₃—, and R⁴—OC(O)—(CH₂)—.
24b. The composition of item 24 or 24a, wherein the conjugate has formula (III) and R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.
24c. The composition of item 24, wherein the conjugate has formula (III′) and R² is selected from the group consisting of R⁴C(O)NH—, R⁴S—, R⁴S(O)₂—, and R⁴NH-(3,4-dioxocyclobut-1-en-1,2-diyl)-NH—.
24d. The composition of item 17 or 24c, wherein the conjugate has formula (III′) and R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, preferably R³ is C₁₋₃ alkyl optionally substituted with one substituent selected from the group consisting of —COOH, —COOCH₃, and —C(O)NH(CH₂)₂NH₂.
25. The composition of any one of items 1 to 23, wherein the conjugate has the general formula (III) or (III′) as shown in item 24, wherein:
   a is an integer between 1 and 2;
   R¹ is methyl or ethyl and is independently selected for each repeating unit;
   m is 10 to 100;
   R², for formula (III), is selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—, wherein each R⁴ is independently a straight hydrocarbyl group having at least 10 carbon atoms; or R², for formula (III′), is R⁴—C(O)(CH₂)₂C(O)NH— or R⁴N—, wherein each R⁴ is independently a straight hydrocarbyl group having at least 10 carbon atoms or a tocopherol moiety, wherein one R⁴ in the same R² moiety is a tocopherol moiety; and
   R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
26. The composition of any one of items 3 to 25, wherein a is 1.
27. The composition of any one of items 3 to 25, wherein a is 2.
28. The composition of item 1 or 2, wherein the POX and/or POZ polymer is a copolymer comprising repeating units of the following general formulas (Ia) and (Ib):

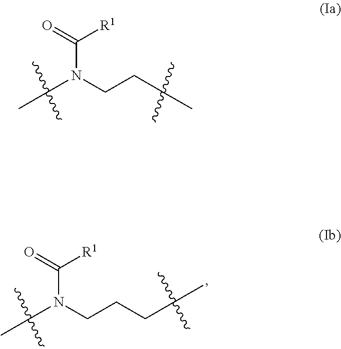

wherein each of R¹ is independently alkyl, in particular C₁₋₃ alkyl, such as methyl, ethyl, iso-propyl, or n-propyl, and is independently selected for each repeating unit; the number of repeating units of formula (Ia) in the copolymer is 1 to 199; the number of repeating units of formula (Ib) in the copolymer is 1 to 199; and the sum of the number of repeating units of formula (Ia) and the number of repeating units of formula (Ib) in the copolymer is 2 to 200.
29. The composition of any one of items 1, 2, and 28, wherein the conjugate has the following general formula (IV):

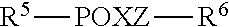

wherein:
R⁵ is R⁷ or -L² (R⁷)_{q}, wherein each R⁷ is independently a hydrocarbyl group; L² is a linker; and
q is 1 or 2;
POXZ is a copolymer containing repeating units of the following general formulas (Ia) and (Ib):

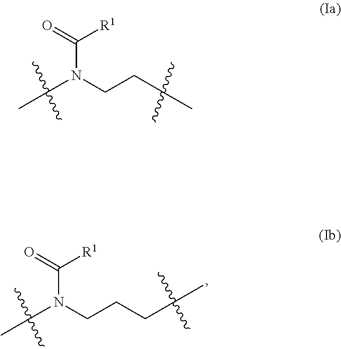

wherein each of R¹ is independently alkyl, in particular C₁₋₃ alkyl, such as methyl, ethyl, iso-propyl, or n-propyl, and is independently selected for each repeating unit; the number of repeating units of formula (Ia) in the copolymer is 1 to 199; the number of repeating units of formula (Ib) in the copolymer is 1 to 199; the sum of the number of repeating units of formula (Ia) and the number of repeating units of formula (Ib) in the copolymer is 2 to 200; and the repeating units of formulas (Ia) and (Ib) are arranged in a random, periodic, alternating or block wise manner; and
R⁶ is selected from the group consisting of H, C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.
29a. The composition of item 29, wherein the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor.
30. The composition of item 28, 29, or 29a, wherein each of R¹ is independently methyl or ethyl, preferably methyl, and is independently selected for each repeating unit.
31. The composition of any one of items 28 to 30, wherein the number of repeating units of formula (Ia) in the copolymer is 1 to 179, such as 1 to 159, 1 to 139, 1 to 119 or 1 to 99; the number of repeating units of formula (Ib) in the copolymer is 1 to 179, such as 1 to 159, 1 to 139, 1 to 119 or 1 to 99; and the sum of the number of repeating units of formula (Ia) and the number of repeating units of formula (Ib) in the copolymer is 2 to 180, such as 4 to 160, 6 to 140, 8 to 120 or 10 to 100.
32. The composition of any one of items 29 to 31, wherein L² comprises at least one functional moiety, such as an alkylene moiety substituted with at least one monovalent functional moiety and/or linked, at the end by which the alkylene group is attached to R⁷, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties.
32a. The composition of any one of items 29 to 32, wherein the conjugate comprises one of the following structures and optionally R⁶ is attached to the terminal C atom of the POXZ copolymer:
   (hydrophobic chain)₁₋₂-(alkylene moiety substituted with at least one monovalent functional moiety)-(POXZ copolymer)
   [(hydrophobic chain)-(divalent functional moiety)]₁₋₂-(alkylene moiety)-(POXZ copolymer).
32b. The composition of any one of items 29 to 32a, wherein the conjugate has one of the following formulas and optionally R⁶ is attached to the terminal C atom of the POXZ copolymer:
   (hydrophobic chain)₁₋₂-(alkylene moiety substituted with at least one monovalent functional moiety)-(POXZ copolymer)-R⁶
   [(hydrophobic chain)-(divalent functional moiety)]₁₋₂-(alkylene moiety)-(POXZ copolymer)-R⁶.
32c. The composition of any one of items 29 to 32b, wherein the alkylene moiety substituted with at least one monovalent functional moiety is substituted with 1, 2, 3, 4, 5, or 6 independently selected monovalent functional moieties.
32d. The composition of any one of items 29 to 32c, wherein the alkylene moiety is C₁₋₆-alkylene, such as C₁₋₃-alkylene, e.g., methylene, ethylene, or trimethylene.
32e. The composition of any one of items 29 to 32, wherein R⁶ is attached to the terminal N atom of the POXZ copolymer and the conjugate comprises one of the following structures:
   (hydrophobic chain)-(divalent functional moiety)-(POXZ copolymer)
   [(hydrophobic chain)-(divalent functional moiety)]₁₋₂-(alkylene moiety)-(divalent functional moiety)-(POXZ copolymer)
   (hydrophobic chain)-(divalent functional moiety)-(cycloalkylene moiety)-(divalent functional moiety)-(POXZ copolymer)
   (hydrophobic chain)-(divalent functional moiety)-(cycloalkenylene moiety)-(divalent functional moiety)-(POXZ copolymer)
   (hydrophobic chain)-(divalent functional moiety)-(alkylene moiety)-(POXZ copolymer)
   [(hydrophobic chain)₂-(trivalent functional moiety)]-(alkylene moiety)-(divalent functional moiety)-(POXZ copolymer)
32f. The composition of any one of items 29 to 32 and 32e, wherein R⁶ is attached to the terminal N atom of the POXZ copolymer and the conjugate has one of the following formulas:
   (hydrophobic chain)-(divalent functional moiety)-(POXZ copolymer)-R⁶
   [(hydrophobic chain)-(divalent functional moiety)]₁₋₂-(alkylene moiety)-(divalent functional moiety)-(POXZ copolymer)-R⁶
   (hydrophobic chain)-(divalent functional moiety)-(cycloalkylene moiety)-(divalent functional moiety)-(POXZ copolymer)-R⁶
   (hydrophobic chain)-(divalent functional moiety)-(cycloalkenylene moiety)-(divalent functional moiety)-(POXZ copolymer)-R⁶
   (hydrophobic chain)-(divalent functional moiety)-(alkylene moiety)-(POXZ copolymer)-R⁶
   [(hydrophobic chain)₂-(trivalent functional moiety)]-(alkylene moiety)-(divalent functional moiety)-(POXZ copolymer)-R⁶
32g. The composition of item 32e or 32f, wherein the alkylene moiety is C₁₋₆-alkylene, such as C₁₋₃-alkylene, e.g., methylene, ethylene, or trimethylene.
32h. The composition of any one of items 32e to 32g, wherein the cycloalkylene moiety is C₃₋₈-cycloalkylene, such as C₃₋₆-cycloalkylene, e.g., cyclopropylene, cyclobutylene, cyclopentylene, cyclohexylene; and/or the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein each of the cycloalkylene and cycloalkenylene moieties is optionally substituted with one or more (e.g., 1, 2, 3, or 4) substituents (e.g., independently selected from the group consisting of —OH, ═O, —SH, halogen, —CN, —N₃, and C₁₋₃-alkyl).
32i. The composition of any one of any one of items 29 to 32, wherein the conjugate comprises the following structure and optionally R⁶ is attached to the terminal C atom of the POXZ copolymer:
   [(hydrophobic chain)₂-(trivalent functional moiety)]-(alkylene moiety)-(POX and/or POZ polymer).
32j. The composition of any one of any one of items 29 to 32 and 32i, wherein the conjugate has the following formula and optionally R⁶ is attached to the terminal C atom of the POXZ copolymer:
   [(hydrophobic chain)₂-(trivalent functional moiety)]-(alkylene moiety)-(POX and/or POZ polymer)-R⁶.
33. The composition of any one of items 29 to 32d, wherein L² is selected from the group consisting of [*—NHC(O)], (C₁₋₆-alkylene)-, [*—C(O)NH], (C₁₋₆-alkylene)-, [*—C(O)O], (C₁₋₆-alkylene)-, [*—OC(O)]_{q}(C₁₋₆-alkylene)-, [*—S], (C₁₋₆-alkylene)-, [*—SS]_{q}(C₁₋₆-alkylene)-, [*—S(O)₂]ₚ(C₁₋₆-alkylene)-, [(*—O)ₛC(OR²⁵)₃₋ₛ](C₁₋₆-alkylene)-, [*—C(OR²⁵)₂O]_{q}(C₁₋₆-alkylene)-, [*—C(R²⁵)(═N—N(R²⁶)C(O)—)]_{q}—(C₁₋₆-alkylene)-, [*—C(O)(N(R²⁶)—N═)C(R²⁵)—]_{q}(C₁₋₆-alkylene)-, [*═C(═N—N(R²⁶)C(O)(R²⁵))]_{q}(C₁₋₆-alkylene)-, [*—N(R²⁶)N(R²⁶)], (C₁₋₆-alkylene)-, [*═C(═N(OH))], (C₁₋₆-alkylene)-, and [*—OC(R²⁵)(R²⁶)O]_{q}(C₁₋₆-alkylene)-, wherein * represents the attachment point to R⁷; q is 1 or 2; C₁₋₆-alkylene is either bivalent (if q is 1) or trivalent (if q is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and s is an integer between 1 and 2.
33a. The composition of any one of items 29 to 32d and 33, wherein L² is selected from the group consisting of [*—NHC(O)], (C₁₋₃-alkylene)-, [*—C(O)NH], (C₁₋₃-alkylene)-, [*—C(O)O], (C₁₋₃-alkylene)-, [*—OC(O)], (C₁₋₃-alkylene)-, [*—S]_{q}(C₁₋₃-alkylene)-, [*—SS]_{q}(C₁₋₃-alkylene)-, [*—S(O)₂]ₚ(C₁₋₃-alkylene)-, [(*—O)ₛC(OR²⁵)₃₋ₛ]—(C₁₋₃-alkylene)-, [*—C(OR²⁵)₂O]_{q}(C₁₋₃-alkylene)-, [*—C(R²⁵)(═N—N(R²⁶)C(O)—)]_{q}(C₁₋₃-alkylene)-, [*—C(O)(N(R²⁶)—N═)C(R²⁵)—]_{q}(C₁₋₃-alkylene)-, [*═C(═N—N(R²⁶)C(O)(R²⁵))]_{q}(C₁₋₃-alkylene)-, [*—N(R²⁶)N(R²⁶)], (C₁₋₃-alkylene)-, [*═C(═N(OH))], (C₁₋₃-alkylene)-, and [*—OC(R²⁵)(R²⁶)O]_{q}—(C₁₋₃-alkylene)-, wherein * represents the attachment point to R⁷; q is 1 or 2; C₁₋₃-alkylene is either bivalent (if q is 1) or trivalent (if q is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and s is an integer between 1 and 2.
33b. The composition of item 33 or 33a, wherein R²⁵ is selected from the group consisting of C₁₋₃ alkyl, phenyl, and phenyl(C₁₋₃ alkyl); and/or R²⁶ is selected from the group consisting of H, C₁₋₃ alkyl, phenyl, and phenyl(C₁₋₃ alkyl).
33c. The composition of any one of items 29 to 32d and 33, wherein L² is selected from the group consisting of [*—NHC(O)], (C₁₋₆-alkylene)-, [*—C(O)NH]_{q}(C₁₋₆-alkylene)-, [*—C(0) O], (C₁₋₆-alkylene)-, [*—OC(O)]_{q}(C₁₋₆-alkylene)-, [*—S], (C₁₋₆-alkylene)-, and [*—S(O)₂]ₚ(C₁₋₆-alkylene)-, preferably from the group consisting of [*—NHC(O)], (C₁₋₆-alkylene)-, [*—C(O)O]_{q}(C₁₋₆-alkylene)-, [*—OC(O)]_{q}(C₁₋₆-alkylene)-, [*—S], (C₁₋₆-alkylene)-, and [*—S(O)₂]ₚ(C₁₋₆-alkylene)-, more preferably from the group consisting of [*—NHC(O)], (C₁₋₆-alkylene)- and [*—C(O)O], (C₁₋₆-alkylene)-, wherein * represents the attachment point to R⁷; q is 1 or 2; and C₁₋₆-alkylene is either bivalent (if q is 1) or trivalent (if q is 2).
33d. The composition of item 33c, wherein L² is selected from the group consisting of [*—NHC(O)]_{q}(C₁₋₃-alkylene)-, [*—C(O)NH], (C₁₋₃-alkylene)-, [*—C(O)O], (C₁₋₃-alkylene)-, [*—OC(O)]_{q}(C₁₋₃-alkylene)-, [*—S], (C₁₋₃-alkylene)-, and [*—S(O)₂]ₚ(C₁₋₃-alkylene)-, preferably from the group consisting of [*—NHC(O)], (C₁₋₃-alkylene)-, [*—C(O)O]_{q}(C₁₋₃-alkylene)-, [*—OC(O)]_{q}(C₁₋₃-alkylene)-, [*—S]_{q}(C₁₋₃-alkylene)-, and [*—S(O)₂]ₚ(C₁₋₃-alkylene)-, more preferably from the group consisting of [*—NHC(O)], (C₁₋₃-alkylene)- and [*—C(O)O]_{q}(C₁₋₃-alkylene)-, wherein * represents the attachment point to R⁷; q is 1 or 2; and C₁₋₃-alkylene is either bivalent (if q is 1) or trivalent (if q is 2).
34. The composition of any one of items 29 to 32d and 33 to 33d, wherein L² is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R⁷.
35. The composition of any one of items 29 to 32d, 33 to 33d, and 34, wherein R⁵ is selected from the group consisting of R⁷, -L²R⁷, —(CH₂)—CH(OC(O)R⁷)(CH₂OC(O)R⁷), —(CH₂)—CH(SR⁷)₂, and —(CH₂)—CH(SR⁷)—CH₂(SR⁷); and L² is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L² is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁷.
35a. The composition of any one of items 29 to 32d and 33 to 35, wherein R⁵ is-L² (R⁷)_{q}.
36. The composition of any one of items 29 to 32, 32i, and 32j, wherein L² is selected from the group consisting of [*-]₂NC(O)(C₁₋₆-alkylene)-, [*—C(O)O], (C₁₋₆-alkylene)-OP(O)₂O(C₁₋₆-alkylene) NHC(O)—(C₁₋₆-alkylene)-, [*—OC(O)]_{q}(C₁₋₆-alkylene)-OP(O)₂O(C₁₋₆-alkylene) NHC(O)—(C₁₋₆-alkylene)-, and [*—C(O)], (C₁₋₆-alkylene)-C(O)—, wherein * represents the attachment point to R⁷; q is 1 or 2; and C₁₋₆-alkylene is either bivalent (if q is 1) or trivalent (if q is 2).
37. The composition of any one of items 29 to 32, 32i, 32j, and 36, wherein L² is selected from the group consisting of [*-]₂NC(O)(C₁₋₂-alkylene)-, [*—OC(O)]₂ (C₂₋₄-alkylene)-OP(O)₂O(C₂₋₃-alkylene) NHC(O)—(C₁₋₂-alkylene)-, and *—C(O)(C₂₋₃-alkylene)-C(O)—, wherein * represents the attachment point to R⁷.
38. The composition of any one of items 29 to 32, 32i, 32j, 36, and 37, wherein R⁵ is selected from the group consisting of (R⁷)₂NC(O)(C₁₋₂-alkylene)-, R⁷C(O)OCH₂CH(OC(O)R⁷)—CH₂—OP(O)₂O—(CH₂)₂—NHC(O)—CH₂—, and R⁷—C(O)(CH₂)₂—C(O)—.
38a. The composition of any one of items 32i, 32j, and 36 to 38, wherein R⁵ is-L¹(R⁷)_{q}.
39. The composition of any one of items 29 to 32 and 32e to 32h, wherein R⁶ is attached to the N-end of the POXZ copolymer; and L² is attached to the C-end of the POXZ copolymer and is selected from the group consisting of [*—Z¹], (C₁₋₆-alkylene)-Z¹—, *—Z¹—(C₃₋₈-cycloalkylene)-Z¹—, *—Z¹—(C₃₋₈-cycloalkenylene)-Z¹—, (*═N)(C₁₋₆-alkylene)-Z¹—, *—Z¹ (C₁₋₆-alkylene)-, and *—Z¹—, wherein * represents the attachment point to R⁷; q is 1 or 2; C₁₋₆-alkylene is either bivalent (if q is 1) or trivalent (if q is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(C₁₋₃-alkylene) NH—, —NH(C₁₋₃-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—.
39a. The composition of any one of items 29 to 32, 32e to 32h, and 39, wherein R⁶ is attached to the N-end of the POXZ copolymer; and L² is attached to the C-end of the POXZ copolymer and is selected from the group consisting of [*—Z¹], (C₁₋₃-alkylene)-Z¹—, *—Z¹—(C₃₋₆-cycloalkylene)-Z¹—, *—Z¹—(C₃₋₆-cycloalkenylene)-Z¹—, (*═N)(C₁₋₃-alkylene)-Z¹—, *—Z¹ (C₁₋₃-alkylene)-, and *—Z¹—, wherein * represents the attachment point to R⁷; q is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(C₁₋₂-alkylene) NH—, —NH(C₁₋₂-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—.
39b. The composition of any one of items 29 to 32, 32e to 32h, 39, and 39a, wherein R⁶ is attached to the N-end of the POXZ copolymer; and L¹ is attached to the C-end of the POXZ copolymer and is selected from the group consisting of [*—Z¹], (C₁₋₃-alkylene)-Z¹—, *—Z¹—(C₃₋₆-cycloalkylene)-Z¹—, *—Z¹—(C₃₋₆-cycloalkenylene)-Z¹—, (*═N)(C₁₋₃-alkylene)-Z¹—, *—Z¹ (C₁₋₃-alkylene)-, and *—Z¹—, wherein * represents the attachment point to R⁷; p is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—.
39c. The composition of any one of items 29 to 32, 32e to 32h, 39, 39a, and 39b, wherein R⁶ is attached to the N-end of the POXZ copolymer; and L² is attached to the C-end of the POXZ copolymer and is selected from the group consisting of (*—C(O)O)(CH(OC(O)—*))(CH₂)—Z¹—, (*═N)(C₁₋₃-alkylene)-NHC(O)—, (*—Z¹ (C₁₋₃-alkylene)-Z¹—, *—Z¹—(C₃₋₆-cycloalkenylene)-Z¹—, and *—Z¹—, wherein * represents the attachment point to R⁷; the C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
39d. The composition of any one of items 29 to 32, 32e to 32h, and 39 to 39c, wherein R⁶ is attached to the N-end of the POXZ copolymer; and R⁵ is attached to the C-end of the POXZ copolymer and is selected from the group consisting of (R⁴C(O)O)(CH(OC(O)R⁷))(CH₂)—Z¹—, (R⁷)₂N(C₁₋₃-alkylene)-Z¹—, R⁷Z¹ (C₁₋₃-alkylene)-Z¹—, R⁷Z¹—(C₃₋₆-cycloalkenylene)-Z¹—, and R⁷Z¹—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
39e. The composition of any one of items 32e to 32h and 39 to 39d, wherein R⁵ is-L² (R⁷)_{q}.
39f. The composition of any one of items 29 to 39, wherein R⁵ is-L² (R⁷)_{q}.
40. The composition of any one of items 29 to 32, wherein R⁶ is attached to the N-end of the POXZ copolymer; and L² is attached to the C-end of the POXZ copolymer and is selected from the group consisting of [*—C(O)], (C₁₋₆-alkylene)-C(O)NH— and [*-]₂N—, wherein * represents the attachment point to R⁷; q is 1 or 2; and C₁₋₆-alkylene is either bivalent (if q is 1) or trivalent (if q is 2).
41. The composition of any one of items 29 to 32 and 40, wherein R⁶ is attached to the N-end of the POXZ copolymer; and L² is attached to the C-end of the POXZ copolymer and is *—C(O)(C₂₋₃-alkylene)-C(O)NH— or [*-]₂N—, wherein * represents the attachment point to R⁷.
42. The composition of any one of items 29 to 32, 40, and 41, wherein R⁶ is attached to the N-end of the POXZ copolymer; and R⁵ is attached to the C-end of the POXZ copolymer and is R⁷—C(O)(CH₂)₂C(O)NH— or R⁷N—.
42a. The composition of any one of items 40 to 42, wherein R⁵ is-L² (R⁷)_{q}.
43. The composition of any one of items 29 to 39f, wherein each R⁷ is independently a non-cyclic, preferably straight hydrocarbyl group.
44. The composition of any one of items 29 to 43, wherein each R⁷ is independently a hydrocarbyl group having at least 8 carbon atoms, such as at least 10 carbon atoms.
44a. The composition of any one of items 29 to 44, wherein each R⁷ is independently a hydrocarbyl group having at most 16 carbon atoms, such as at most 14 carbon atoms or at most 12 carbon atoms.
44b. The composition of any one of items 29 to 44, wherein R⁵ is attached to the C-end of the POXZ copolymer and each R⁷ is independently a hydrocarbyl group having 10 to 18 carbon atoms, such as a straight alkyl group having 10 to 18 carbon atoms or a straight alkenyl group having 10 to 18 carbon atoms.
45. The composition of any one of items 29 to 42a and 44, wherein one R⁷ is a tocopherol moiety.
46. The composition of any one of items 29 to 44b and 45, wherein R⁶ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.
47. The composition of any one of items 29 to 46, wherein R⁶ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, —C(O)NR²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group.
48. The composition of any one of items 29 to 47, wherein R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
49. The composition of any one of items 1, 2, and 28 to 48, wherein the conjugate has the following general formula (V):

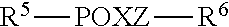

wherein:
R⁵, when attached to the N-end of the POXZ copolymer, is selected from the group consisting of -L²R⁷, —(CH₂)—CH(OC(O)R⁷)(CH₂OC(O)R⁷), —(CH₂)—CH(SR⁷)₂, and —(CH₂)—CH(SR⁷)—CH₂(SR⁷), wherein each R⁷ is independently a straight hydrocarbyl group having at least 10 carbon atoms; and L² is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L² is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁷, or R⁵, when attached to the C-end of the POXZ is from copolymer, selected the group consisting of (R⁷C(O)O)(CH(OC(O)R⁷))(CH₂)—Z¹—, (R⁷)₂N(C₁₋₃-alkylene)-Z¹—, R⁷Z¹ (C₁₋₃-alkylene)-Z¹—, R⁷Z¹—(C₃₋₆-cycloalkenylene)-Z¹—, and R⁷Z¹, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—;
POXZ is a copolymer containing the repeating units of the following general formulas (Ia) and (Ib):

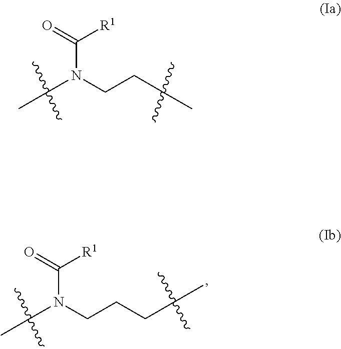

wherein each of R¹ is independently methyl or ethyl and is independently selected for each repeating unit; the number of repeating units of formula (Ia) in the copolymer is 1 to 99; the number of repeating units of formula (Ib) in the copolymer is 1 to 99; the sum of the number of repeating units of formula (Ia) and the number of repeating units of formula (Ib) in the copolymer is 10 to 100; and the repeating units of formulas (Ia) and (Ib) are arranged in a random, periodic, alternating or block wise manner; and
R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
49a. The composition of item 49, wherein R⁵ is attached to the N-end of the POXZ copolymer and is selected from the group consisting of R⁷—NHC(O)—(CH₂)—, R⁷—NHC(O)—(CH₂)₂—, —(CH₂)—CH(OC(O)R⁷)(CH₂OC(O)R⁷), —(CH₂)—CH(SR⁷)—CH₂(SR⁷), R⁷S—(CH₂)₃—, R⁷S(O)₂—(CH₂)₃—, and R⁷—OC(O)—(CH₂)—.
49b. The composition of item 49 or 49a, wherein R⁵ is attached to the N-end of the POXZ copolymer; and R⁶ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.
49c. The composition of item 49, wherein R⁵ is attached to the C-end of the POXZ copolymer and is selected from the group consisting of RIC(O)NH—, R'S—, R'S(O)₂—, and R⁷NH-(3,4-dioxocyclobut-1-en-1,2-diyl)-NH—.
49d. The composition of item 49 or 49c, wherein R⁵ is attached to the C-end of the POXZ copolymer; and R⁶ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, preferably R⁶ is C₁₋₃ alkyl optionally substituted with one substituent selected from the group consisting of —COOH, —COOCH₃, and —C(O)NH(CH₂)₂NH₂.
50. The composition of any one of items 1, 2, and 28 to 48, wherein the conjugate has the general formula (V) as shown in item 49, wherein:
   R⁵, when attached to the N-end of the POXZ copolymer, is selected from the group consisting of (R⁷)₂NC(O)(C₁₋₂-alkylene)-, R⁷C(O)OCH₂CH(OC(O)R⁷)—CH₂—OP(O)₂O—(CH₂)₂—NHC(O)—CH₂—, and R⁷—C(O)(CH₂)₂—C(O)—, wherein each R⁷ is independently a straight hydrocarbyl group having at least 10 carbon atoms, or R⁵, when attached to the C-end of the POXZ copolymer, is R¹—C(O)(CH₂)₂C(O)NH— or R⁷N—, wherein each R⁷ is independently a straight hydrocarbyl group having at least 10 carbon atoms or a tocopherol moiety, wherein one R⁷ in the same R⁵ moiety is a tocopherol moiety; POXZ is a copolymer containing the repeating units of the general formulas (Ia) and (Ib) as shown in item 49, wherein each of R¹ is independently methyl or ethyl and is independently selected for each repeating unit; the number of repeating units of formula (Ia) in the copolymer is 1 to 99; the number of repeating units of formula (Ib) in the copolymer is 1 to 99; the sum of the number of repeating units of formula (Ia) and the number of repeating units of formula (Ib) in the copolymer is 10 to 100; and the repeating units of formulas (Ia) and (Ib) are arranged in a random, periodic, alternating or block wise manner; and
   R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
51. The composition of any one of items 1 to 49d and 50, wherein the conjugate of (a) a POX and/or POZ polymer and (b) one or more hydrophobic chains inhibits aggregation of the LNPs.
52. The composition of any one of items 1 to 51, wherein the conjugate of (a) a POX and/or POZ polymer and (b) one or more hydrophobic chains comprises from about 0.25 mol % to about 50 mol % of the total lipid present in the LNPs.
53. The composition of any one of items 1 to 52, wherein the composition is substantially free of a lipid or lipid-like material comprising polyethyleneglycol (PEG), preferably is substantially free of PEG.
54. The composition of any one of items 1 to 53, wherein the LNPs are non-viral particles.
55. The composition of any one of items 1 to 54, wherein the cationically ionizable lipid comprises a head group which includes at least one nitrogen atom which is capable of being protonated under physiological conditions.
56. The composition of any one of items 1 to 55, wherein the cationically ionizable lipid has the structure of Formula (X):

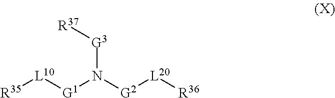

or a pharmaceutically acceptable salt, tautomer, prodrug or stereoisomer thereof, wherein:
one of L¹⁰ and L²⁰ is-O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O—, and the other of L¹⁰ and L²⁰ is-O(C═O)—, —(C═O)O—, —C(═O)—, —O—, —S(O)ₓ—, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆-24 alkyl or C₆₋₂₄ alkenyl;
R³⁷ is H, OR⁵⁰, CN, —C(═O)OR⁴⁰, —OC(═O)R⁴⁰ or —NR⁵⁰C(═O)R⁴⁰;
R⁴⁰ is C₁₋₁₂ alkyl;
R⁵⁰ is H or C₁₋₆ alkyl; and
x is 0, 1 or 2.
57. The composition of any one of items 1 to 55, wherein:
   (α) the cationically ionizable lipid is selected from the following structures X-1 to X-36:

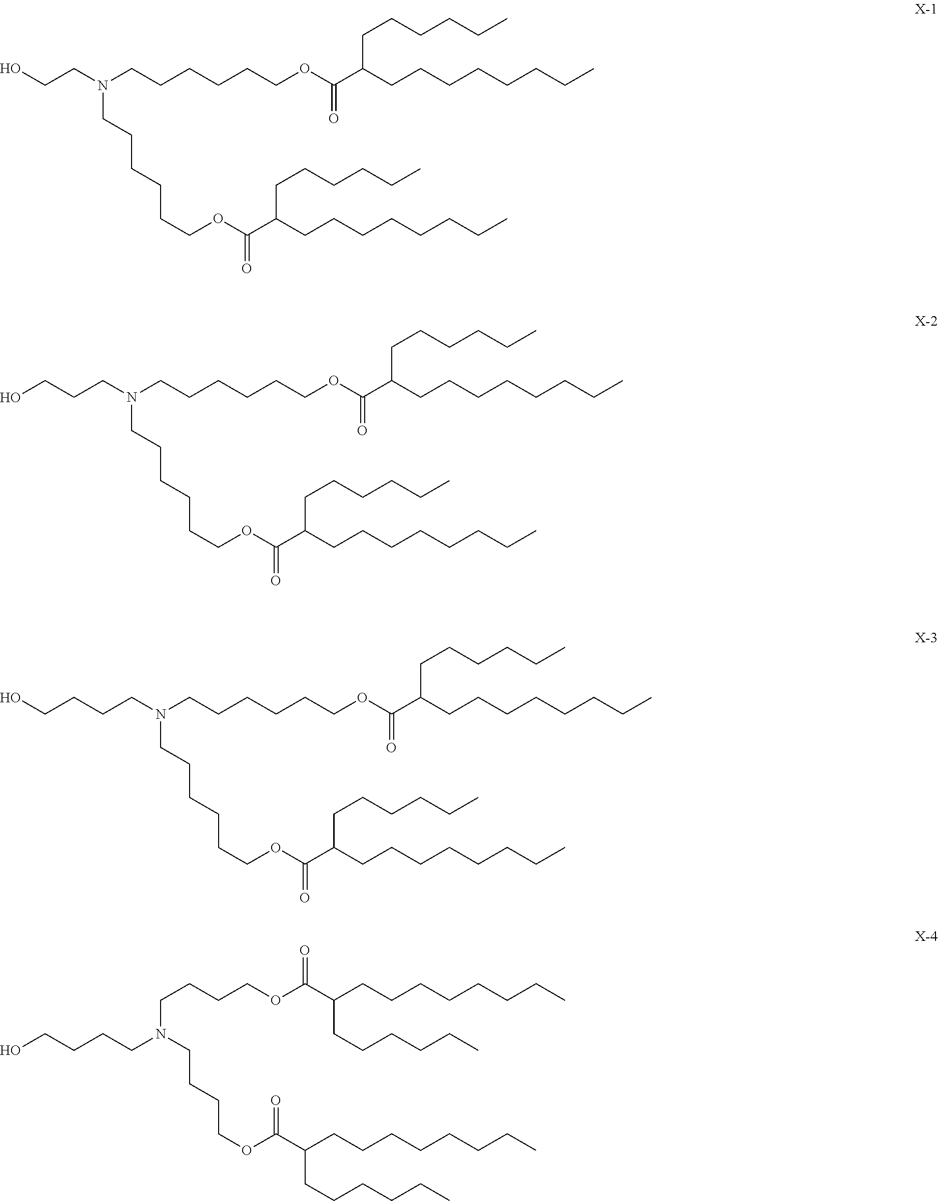 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCN(CCCCO)CCCCOC(=O)C(CCCCCC)CCCCCCCC]

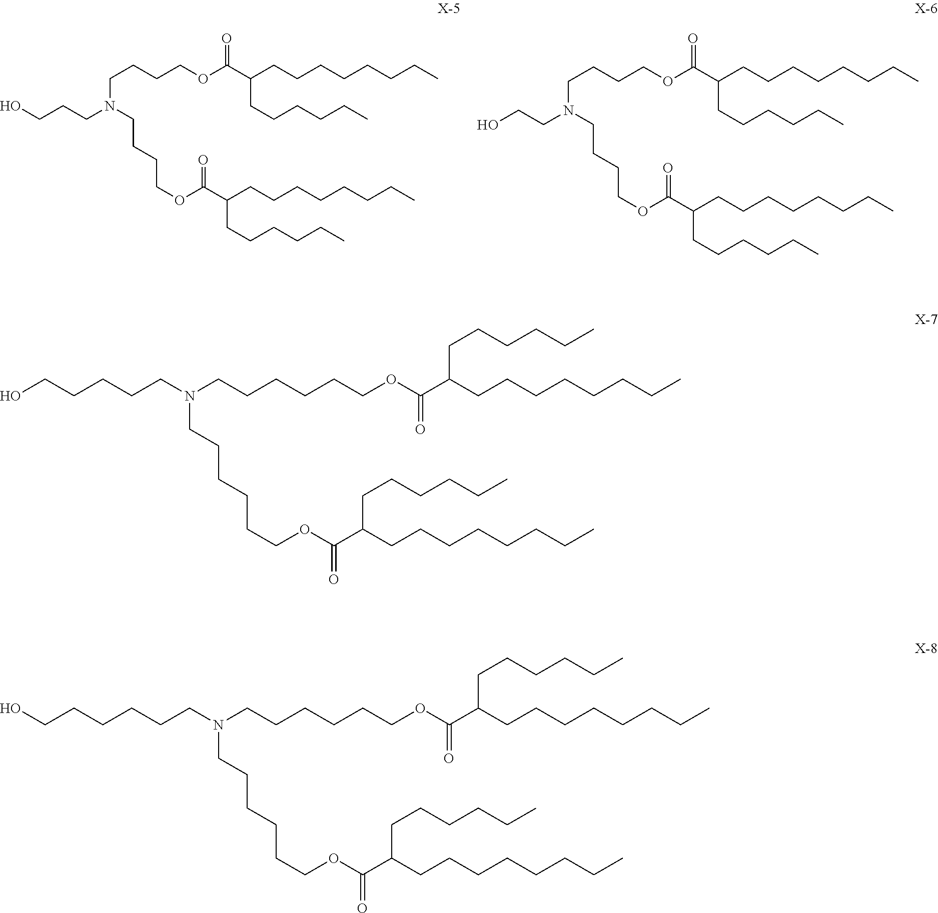 [SMILES: CCCCCCCCC(CCCCCC)C(=O)OCCCCN(CCCO)CCCCOC(=O)C(CCCCCC)CCCCCCCC ; CCCCCCCCC(CCCCCC)C(=O)OCCCCN(CCO)CCCCOC(=O)C(CCCCCC)CCCCCCCC ; CCCCCCCCC(CCCCCC)C(=O)OCCCCCCN(CCCCCO)CCCCCCOC(=O)C(CCCCCC)CCCCCCCC ; CCCCCCCCC(CCCCCC)C(=O)OCCCCCCN(CCCCCCO)CCCCCCOC(=O)C(CCCCCC)CCCCCCCC]

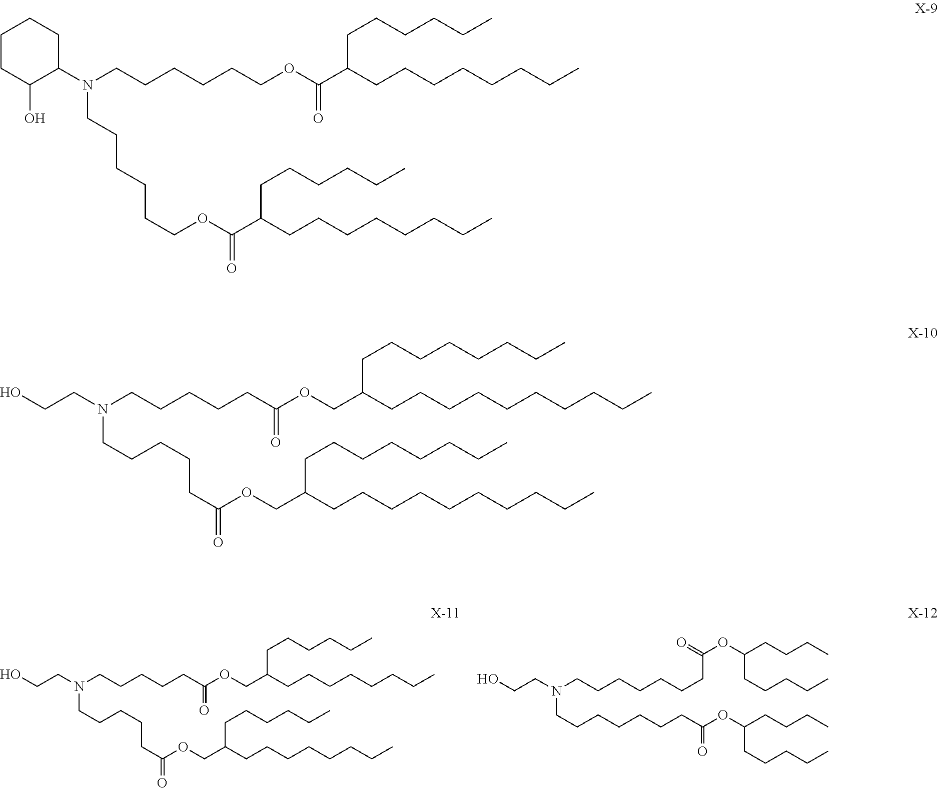 [SMILES: CCCCCCCCC(CCCCCC)C(=O)OCCCCCCN(CCCCCCOC(=O)C(CCCCCC)CCCCCCCC)C1CCCCC1O ; CCCCCCCCCCC(CCCCCCCC)COC(=O)CCCCCN(CCO)CCCCCC(=O)OCC(CCCCCCCC)CCCCCCCCCC ; CCCCCCCCC(CCCCCC)COC(=O)CCCCCN(CCO)CCCCCC(=O)OCC(CCCCCC)CCCCCCCC ; CCCCCC(CCCC)OC(=O)CCCCCCCN(CCO)CCCCCCCC(=O)OC(CCCC)CCCCC]

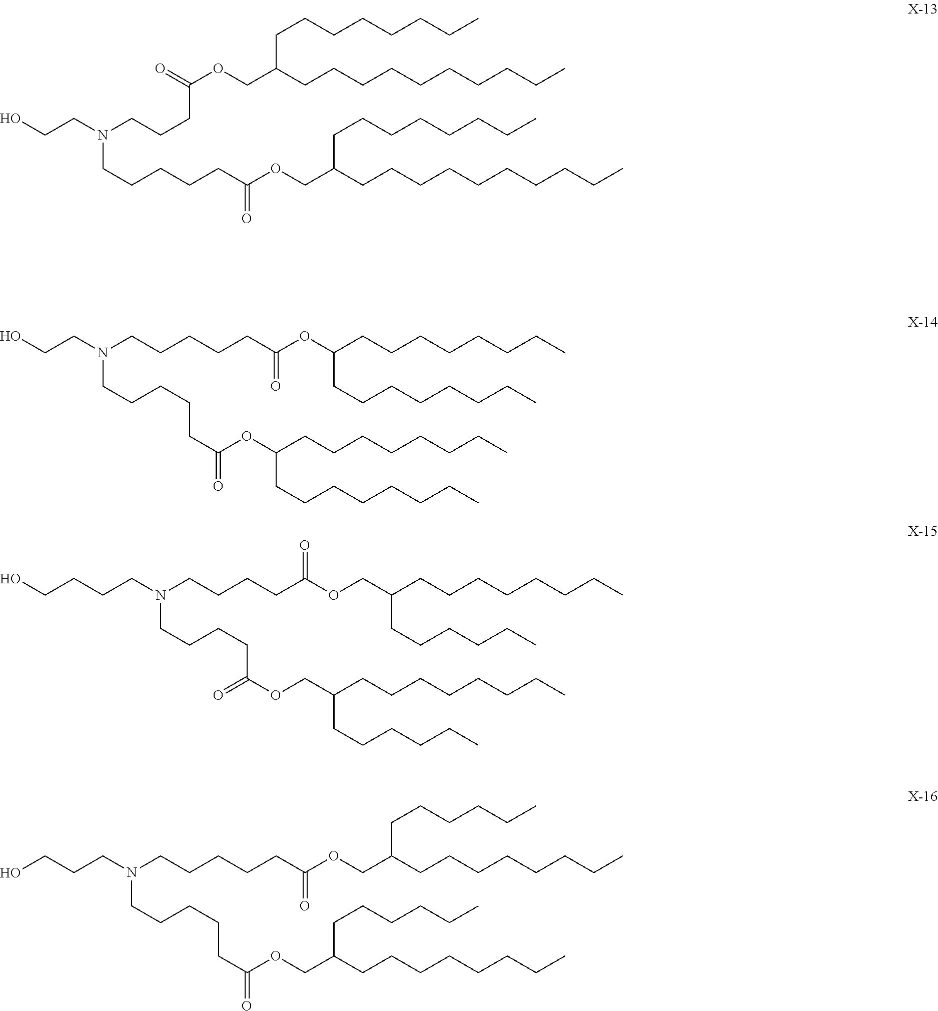

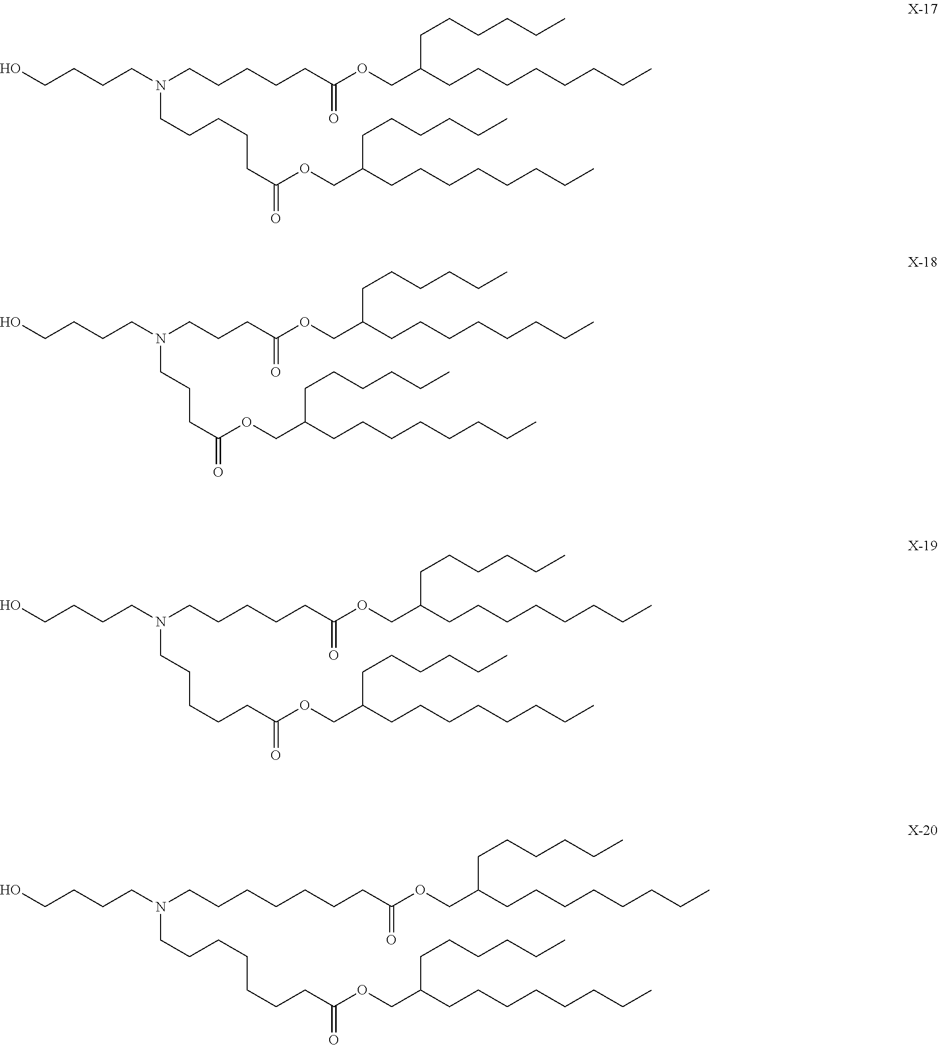 [SMILES: CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCN(CCCCO)CCCC(=O)OCC(CCCCCC)CCCCCCCC ; CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCCCCCN(CCCCO)CCCCCCCC(=O)OCC(CCCCCC)CCCCCCCC]

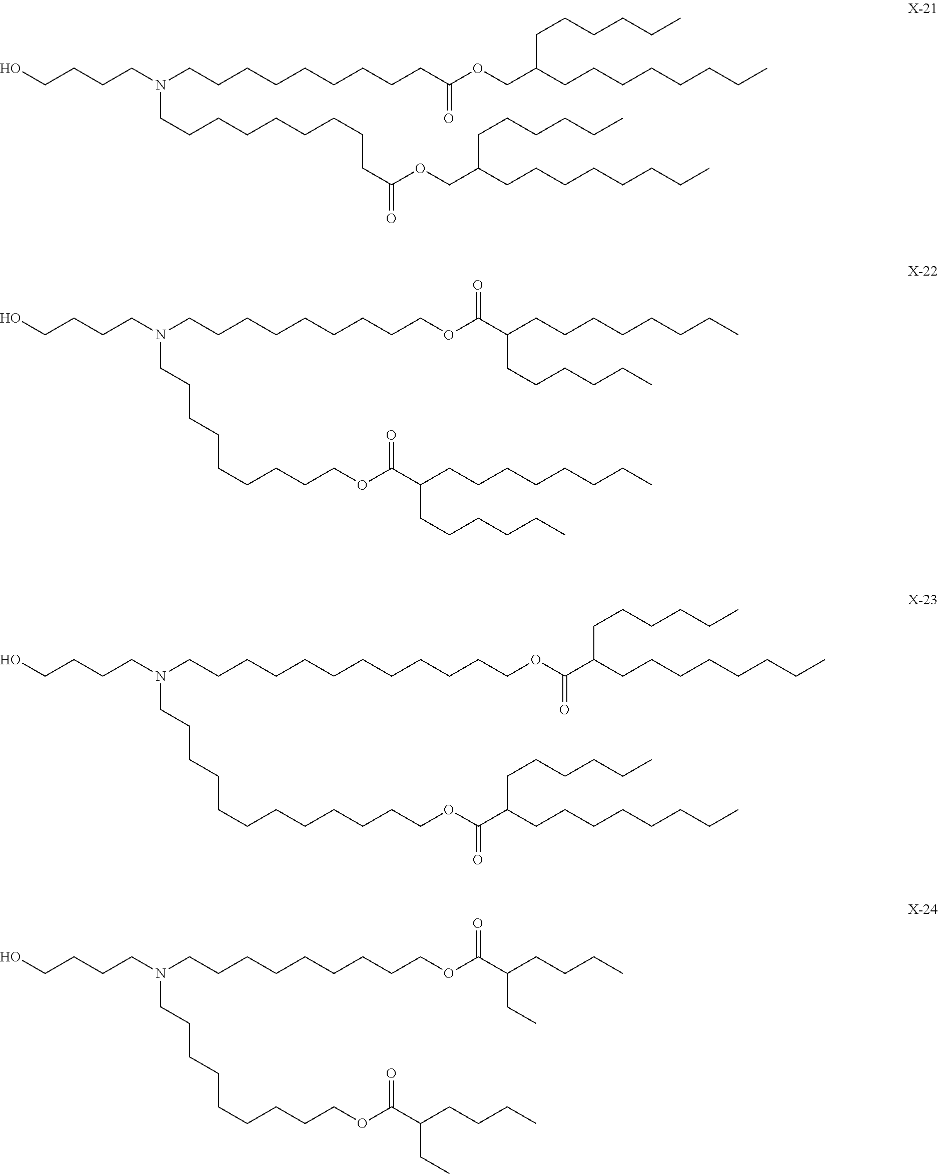 [SMILES: CCCCCCCCC(CCCCCC)COC(=O)CCCCCCCCCN(CCCCO)CCCCCCCCCC(=O)OCC(CCCCCC)CCCCCCCC ; CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCCCCCCCN(CCCCO)CCCCCCCCCCCCOC(=O)C(CCCCCC)CCCCCCCC ; CCCCC(CC)C(=O)OCCCCCCCCCN(CCCCO)CCCCCCCCCOC(=O)C(CC)CCCC]

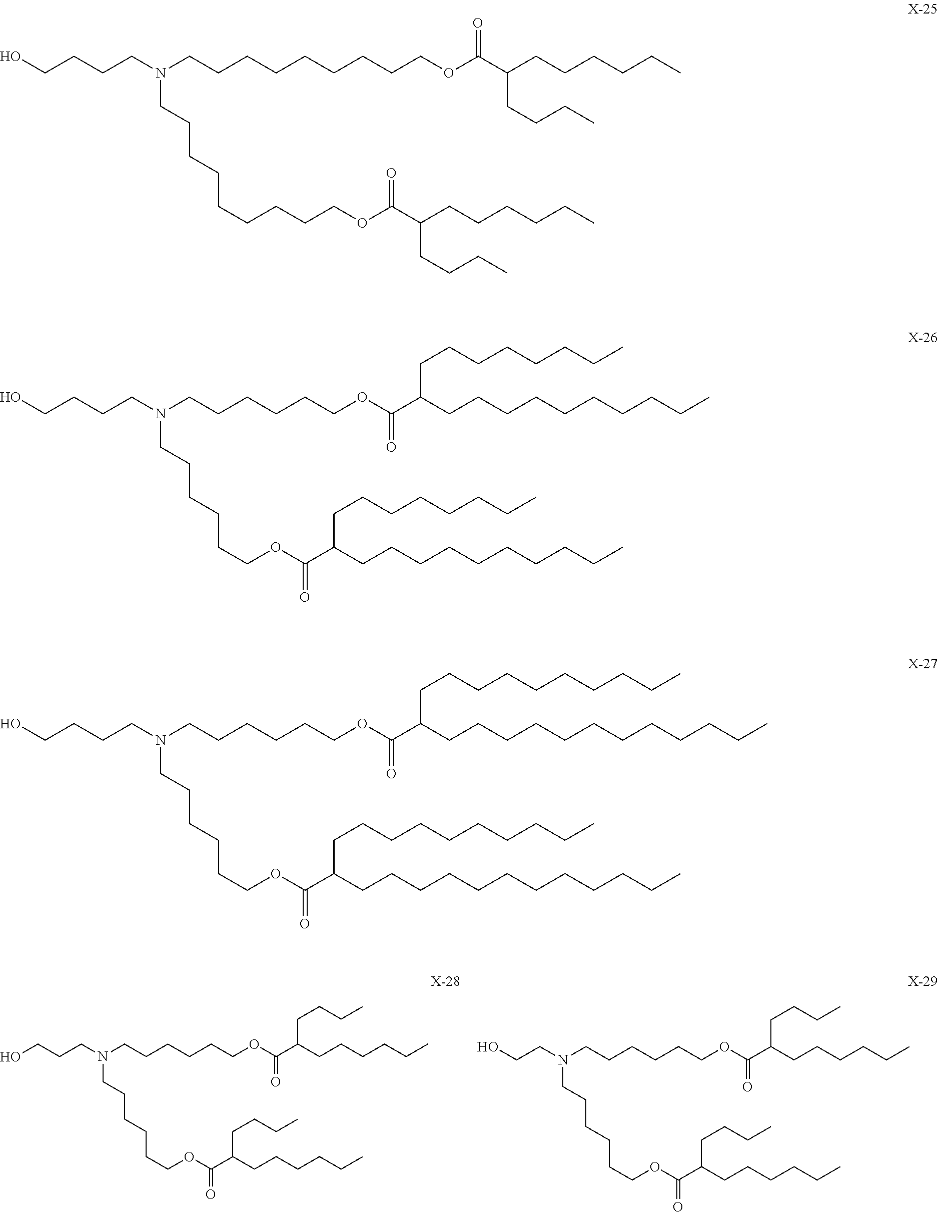 [SMILES: CCCCCCC(CCCC)C(=O)OCCCCCCCCCN(CCCCO)CCCCCCCCCOC(=O)C(CCCC)CCCCCC ; CCCCCCCCCCC(CCCCCCCC)C(=O)OCCCCCCN(CCCCO)CCCCCCOC(=O)C(CCCCCCCC)CCCCCCCCCC ; CCCCCCCCCCCCC(CCCCCCCCCC)C(=O)OCCCCCCN(CCCCO)CCCCCCOC(=O)C(CCCCCCCCCC)CCCCCCCCCCCC ; CCCCCCC(CCCC)C(=O)OCCCCCCN(CCCO)CCCCCCOC(=O)C(CCCC)CCCCCC ; CCCCCCC(CCCC)C(=O)OCCCCCCN(CCO)CCCCCCOC(=O)C(CCCC)CCCCCC]

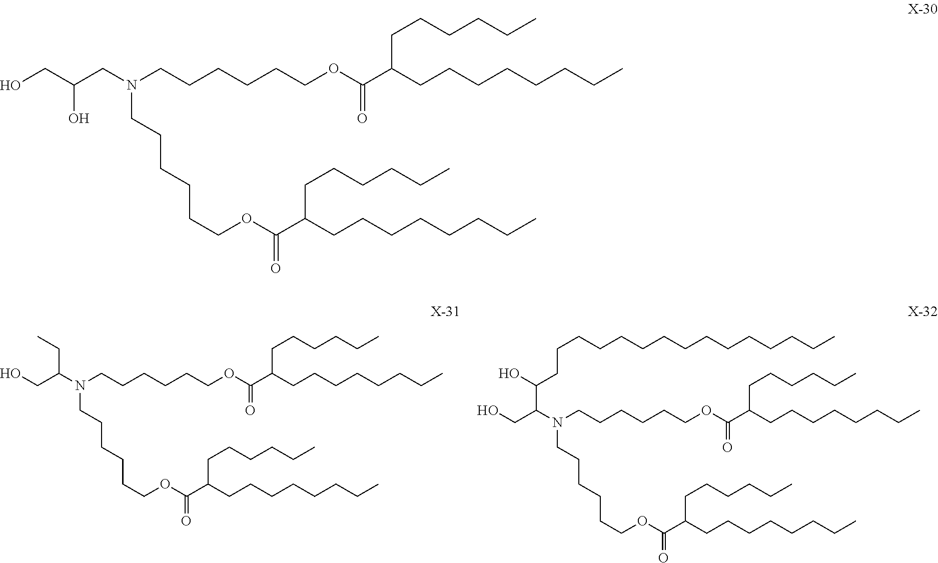 [SMILES: CCCCCCCCC(CCCCCC)C(=O)OCCCCCCN(CCCCCCOC(=O)C(CCCCCC)CCCCCCCC)CC(O)CO ; CCCCCCCCC(CCCCCC)C(=O)OCCCCCCN(CCCCCCOC(=O)C(CCCCCC)CCCCCCCC)C(CC)CO ; CCCCCCCCCCCCCCCC(O)C(CO)N(CCCCCCOC(=O)C(CCCCCC)CCCCCCCC)CCCCCCOC(=O)C(CCCCCC)CCCCCCCC]

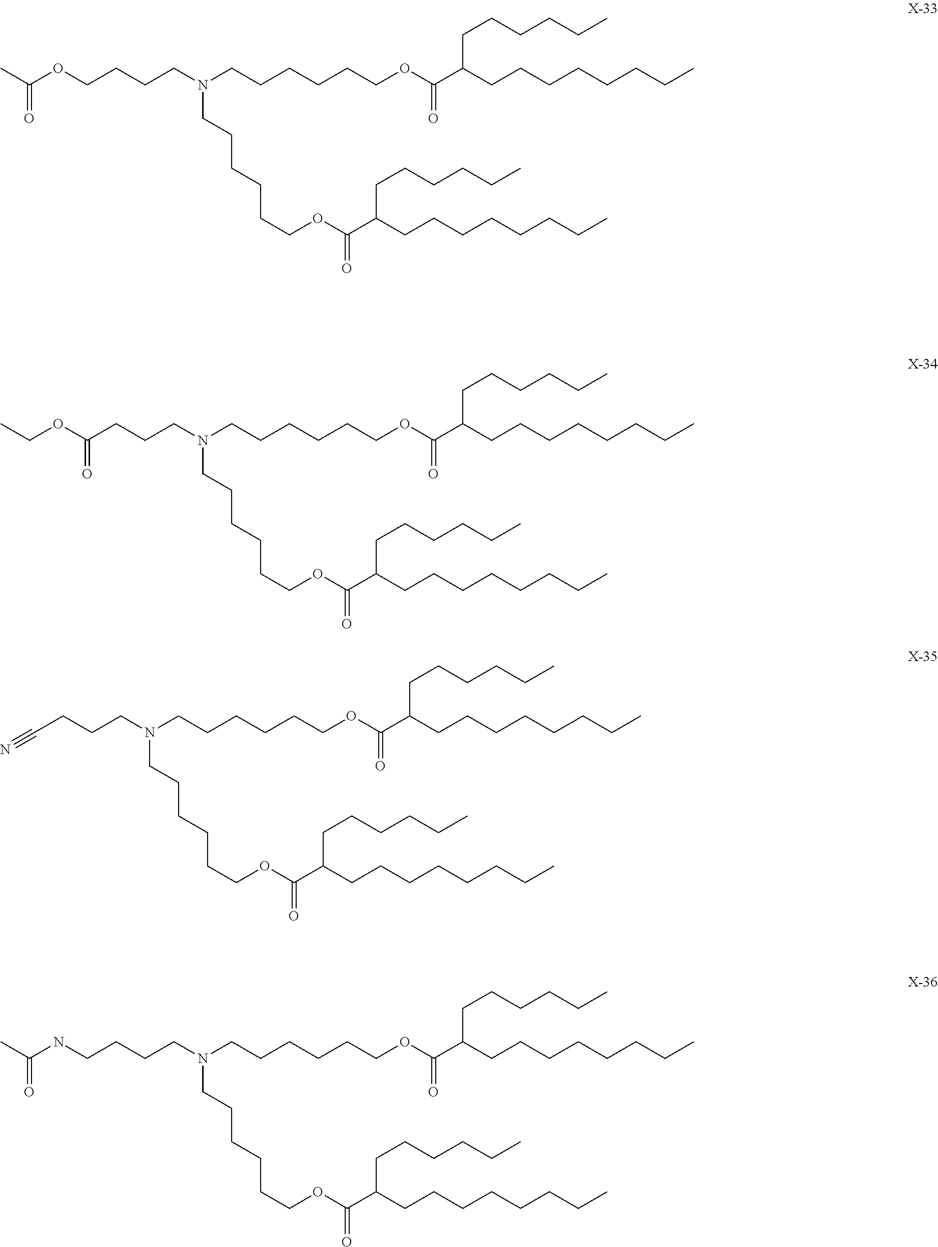

(β) the cationically ionizable lipid is selected from the following structures A to G:

| No. | Structure |
|---|---|
| A | 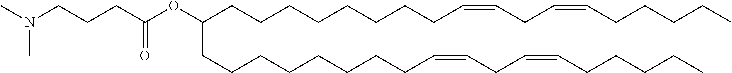 [SMILES: CCCCC/C=C\C/C=C\CCCCCCCCC(CCCCCCCC/C=C\C/C=C\CCCCC)OC(=O)CCCN(C)C] |
| B | 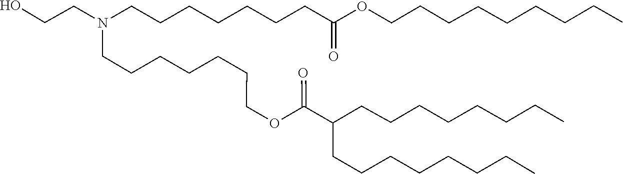 [SMILES: CCCCCCCCCOC(=O)CCCCCCCN(CCO)CCCCCCCOC(=O)C(CCCCCCCC)CCCCCCCC] |
| C | 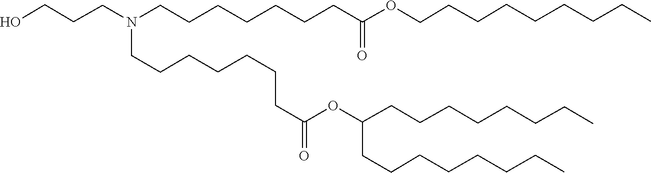 [SMILES: CCCCCCCCCOC(=O)CCCCCCCN(CCCO)CCCCCCCC(=O)OC(CCCCCCCC)CCCCCCCC] |
| D | 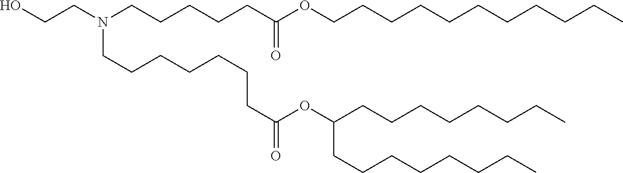 [SMILES: CCCCCCCCCCCOC(=O)CCCCCN(CCO)CCCCCCCC(=O)OC(CCCCCCCC)CCCCCCCC] |
| E | 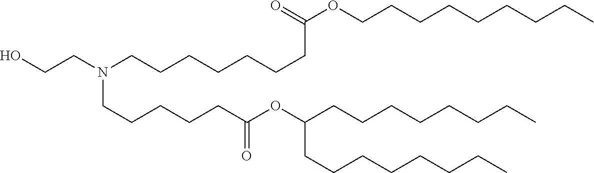 [SMILES: CCCCCCCCCOC(=O)CCCCCCCN(CCO)CCCCCC(=O)OC(CCCCCCCC)CCCCCCCC] |
| F | 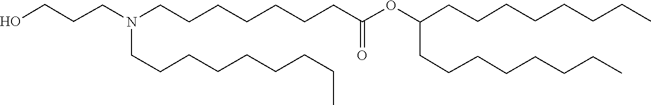 [SMILES: CCCCCCCCCN(CCCO)CCCCCCCC(=O)OC(CCCCCCCC)CCCCCCCC] |
| G | 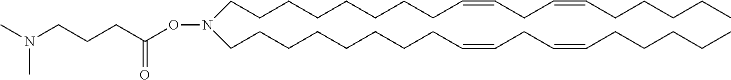 [SMILES: CCCCC/C=C\C/C=C\CCCCCCCCN(CCCCCCCC/C=C\C/C=C\CCCCC)OC(=O)CCCN(C)C] |

or
(γ) the cationically ionizable lipid is the lipid having the structure X-3.
58. The composition of any one of items 1 to 55, wherein the cationic or cationically ionizable lipid has the structure of Formula (XI):

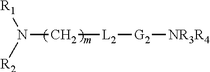

wherein
each of R₁ and R₂ is independently R₅ or -G₁-L₁-R₆, wherein at least one of R₁ and R₂ is -G₁-L₁-R₆;
each of R₃ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —C(═O)—, —O—, —S(O)ₓ—, —S—S—, —C(═O)S—, —SC(═O)—, —NRaC(═O)—, —C(═O)NRa—, —NRaC(═O)NRa—, —OC(═O)NRa— and —NRaC(═O)O—;
Ra is H or C₁₋₁₂ alkyl;
m is 0, 1, 2, 3, or 4; and
x is 0, 1 or 2.
59. The composition of any one of items 1 to 55 and 58, wherein the cationically ionizable lipid is selected from the following structures (XIV-1), (XIV-2), and (XIV-3):

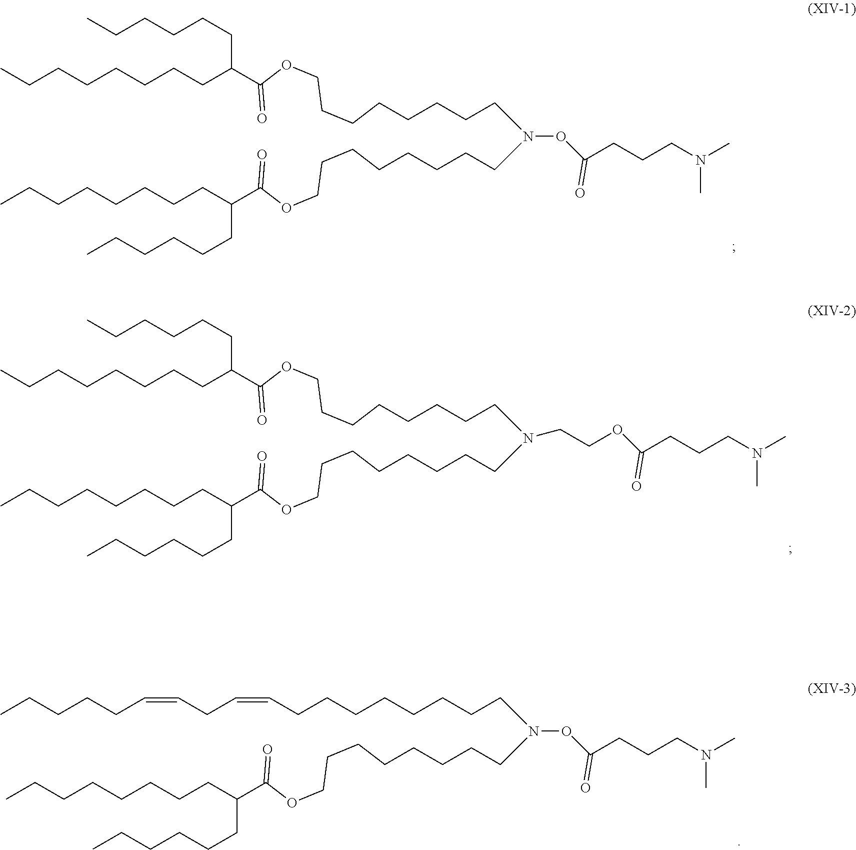

60. The composition of any one of items 1 to 54, wherein the cationic or cationically ionizable lipid comprises 2,3-dioleyloxy-1-(N,N-dimethylamino) propane (DODMA), N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxy) propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), DPL14, or a mixture thereof.
61. The composition of any one of items 1 to 60, wherein the cationic or cationically ionizable lipid comprises from about 20 mol % to about 80 mol % of the total lipid present in the LNPs.
62. The composition of any one of items 1 to 61, wherein the LNPs further comprise one or more additional lipids, preferably selected from the group consisting of phospholipids, steroids, and combinations thereof, more preferably the LNPs comprise the cationically ionizable lipid, the conjugate, a phospholipid, and a steroid.
63. The composition of item 62, wherein the phospholipid is selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dipalmitoylphosphatidylcholine (DPPC), dilauroylphosphatidylcholine, diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₋₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE).
64. The composition of item 62 or 63, wherein the phospholipid comprises from about 5 mol % to about 30 mol % of the total lipid present in the LNPs.
65. The composition of any one of items 62 to 64, wherein the steroid comprises a sterol such as cholesterol.
66. The composition of any one of items 62 to 65, wherein the steroid comprises from about 10 mol % to about 60 mol % of the total lipid present in the LNPs.
67. The composition of any one of items 62 to 66, wherein the cationic or cationically ionizable lipid comprises from about 20 mol % to about 70 mol % of the total lipid present in the LNPs; the conjugate of (a) a POX and/or POZ polymer and (b) one or more hydrophobic chains comprises from about 0.5 mol % to about 15 mol % of the total lipid present in the LNPs; the phospholipid comprises from about 5 mol % to about 25 mol % of the total lipid present in the LNPs; and the steroid comprises from about 25 mol % to about 55 mol % of the total lipid present in the LNPs.
68. The composition of any one of items 1 to 67, wherein the LNPs have a size of from about 30 nm to about 500 nm.
68a. The composition of any one of items 1 to 68, wherein water is the main component in the composition and/or the total amount of solvent(s) other than water contained in the composition is less than about 0.5% (v/v).
69. The composition of any one of items 1 to 68a, wherein the RNA is encapsulated within or associated with the LNPs.
70. The composition of any one of items 1 to 69, wherein the RNA is mRNA.
71. The composition of any one of items 1 to 70, wherein the RNA comprises a modified nucleoside in place of uridine, wherein the modified nucleoside is preferably selected from pseudouridine (ψ), N1-methyl-pseudouridine (m1ψ), and 5-methyl-uridine (m5U).
72. The composition of any one of items 1 to 71, wherein the RNA comprises at least one of the following, preferably all of the following: a 5′ cap: a 5′ UTR: a 3′ UTR; and a poly-A sequence.
73. The composition of item 72, wherein the poly-A sequence comprises at least 100 A nucleotides, wherein the poly-A sequence preferably is an interrupted sequence of A nucleotides.
74. The composition of item 72 or 73, wherein the 5′ cap is a cap1 or cap2 structure.
75. The composition of any one of items 1 to 74, wherein the RNA encodes one or more peptides or proteins, wherein preferably the one or more peptides or proteins are therapeutic peptides or proteins and/or comprise an epitope for inducing an immune response against an antigen in a subject.
76. A method for delivering RNA to cells of a subject, the method comprising administering to a subject a composition of any one of items 1 to 75.
77. A method for delivering a therapeutic peptide or protein to a subject, the method comprising administering to a subject a composition of any one of items 1 to 75, wherein the RNA encodes the therapeutic peptide or protein.
78. A method for treating or preventing a disease or disorder in a subject, the method comprising administering to a subject a composition of any one of items 1 to 75, wherein delivering the RNA to cells of the subject is beneficial in treating or preventing the disease or disorder.
79. A method for treating or preventing a disease or disorder in a subject, the method comprising administering to a subject a composition of any one of items 1 to 75, wherein the RNA encodes a therapeutic peptide or protein and wherein delivering the therapeutic peptide or protein to the subject is beneficial in treating or preventing the disease or disorder.
80. The method of any one of items 76 to 79, wherein the subject is a mammal.
81. The method of item 80, wherein the mammal is a human.
82. A conjugate of (a) a polyoxazoline (POX) or polyoxazine (POZ) polymer and (b) one or more hydrophobic chains, wherein the conjugate has the following general formula (II) or (II′):

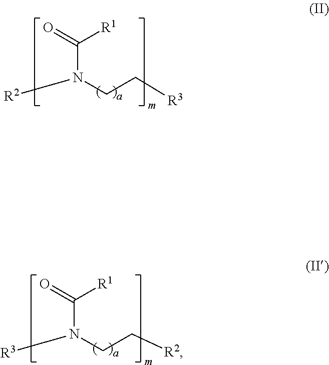

wherein:
a is an integer between 1 and 2;
R¹ is alkyl, in particular C₁₋₃ alkyl, such as methyl, ethyl, iso-propyl, or n-propyl, and is independently selected for each repeating unit;
m is 2 to 200;
R² is R⁴ or -L₁(R⁴)ₚ, wherein each R⁴ is independently a hydrocarbyl group; L₁ is a linker; and
p is 1 or 2; and
R³ is selected from the group consisting of H, C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.
82a. The conjugate of item 82, wherein the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor.
83. The conjugate of item 82 or 82a, wherein R¹ is methyl or ethyl, preferably methyl.
84. The conjugate of item 82, 82a, or 83, wherein m is 2 to 180, such as 4 to 160, 6 to 140, 8 to 120 or 10 to 100.
85. The conjugate of any one of items 82 to 84, wherein L₁ comprises at least one functional moiety, such as an alkylene moiety substituted with at least one monovalent functional moiety and/or linked, at the end by which the alkylene group is attached to R⁴, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.
85a. The conjugate of any one of items 82 to 85, comprising one of the following structures and optionally having the general formula (II):
   (hydrophobic chain)₁₋₂-(alkylene moiety substituted with at least one monovalent functional moiety)-(POX and/or POZ polymer)
[(hydrophobic chain)-(divalent functional moiety)]₁₋₂-(alkylene moiety)-(POX and/or POZ polymer).
85b. The conjugate of any one of items 82 to 85a, having one of the following formulas and optionally having the general formula (II):
   (hydrophobic chain)₁₋₂-(alkylene moiety substituted with at least one monovalent functional moiety)-(POX and/or POZ polymer)-R³
   [(hydrophobic chain)-(divalent functional moiety)]₁₋₂-(alkylene moiety)-(POX and/or POZ polymer)-R³.
85c. The conjugate of any one of items 85 to 85b, wherein the alkylene moiety substituted with at least one monovalent functional moiety is substituted with 1, 2, 3, 4, 5, or 6 independently selected monovalent functional moieties.
85d. The conjugate of any one of items 85 to 85c, wherein the alkylene moiety is C₁₋₆-alkylene, such as C₁₋₃-alkylene, e.g., methylene, ethylene, or trimethylene.
85e. The conjugate of any one of items 82 to 85, having the general formula (II′) and comprising one of the following structures:
   (hydrophobic chain)-(divalent functional moiety)-(POX and/or POZ polymer)
   [(hydrophobic chain)-(divalent functional moiety)]₁₋₂-(alkylene moiety)-(divalent functional moiety)-(POX and/or POZ polymer)
   (hydrophobic chain)-(divalent functional moiety)-(cycloalkylene moiety)-(divalent functional moiety)-(POX and/or POZ polymer)
   (hydrophobic chain)-(divalent functional moiety)-(cycloalkenylene moiety)-(divalent functional moiety)-(POX and/or POZ polymer)
   (hydrophobic chain)-(divalent functional moiety)-(alkylene moiety)-(POX and/or POZ polymer)
   [(hydrophobic chain)₂-(trivalent functional moiety)]-(alkylene moiety)-(divalent functional moiety)-(POX and/or POZ polymer) 85f. The conjugate of any one of items 82 to 85 and 85e, having the general formula (II′) and having one of the following formulas:
   (hydrophobic chain)-(divalent functional moiety)-(POX and/or POZ polymer)-R³
   [(hydrophobic chain)-(divalent functional moiety)]₁₋₂-(alkylene moiety)-(divalent functional moiety)-(POX and/or POZ polymer)-R³
   (hydrophobic chain)-(divalent functional moiety)-(cycloalkylene moiety)-(divalent functional moiety)-(POX and/or POZ polymer)-R³
   (hydrophobic chain)-(divalent functional moiety)-(cycloalkenylene moiety)-(divalent functional moiety)-(POX and/or POZ polymer)-R³
   (hydrophobic chain)-(divalent functional moiety)-(alkylene moiety)-(POX and/or POZ polymer)-R³
   [(hydrophobic chain)₂-(trivalent functional moiety)]-(alkylene moiety)-(divalent functional moiety)-(POX and/or POZ polymer)-R³
85g. The conjugate of item 85e or 85f, wherein the alkylene moiety is C₁₋₆-alkylene, such as C₁₋₃-alkylene, e.g., methylene, ethylene, or trimethylene.
85h. The conjugate of any one of items 85e to 85g, wherein the cycloalkylene moiety is C₃₋₈-cycloalkylene, such as C₃₋₆-cycloalkylene, e.g., cyclopropylene, cyclobutylene, cyclopentylene, cyclohexylene; and/or the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein each of the cycloalkylene and cycloalkenylene moieties is optionally substituted with one or more (e.g., 1, 2, 3, or 4) substituents (e.g., independently selected from the group consisting of —OH, ═O, —SH, halogen, —CN, —N₃, and C₁₋₃-alkyl).
85i. The conjugate of any one of items 82 to 85, comprising the following structure and optionally having the general formula (II):
   [(hydrophobic chain)₂-(trivalent functional moiety)]-(alkylene moiety)-(POX and/or POZ polymer).
85j. The conjugate of any one of items 82 to 85 and 85i, having the following formula and optionally having the general formula (II):
   [(hydrophobic chain)₂-(trivalent functional moiety)]-(alkylene moiety)-(POX and/or POZ polymer)-R³.
86. The conjugate of any one of items 82 to 85d, wherein L₁ is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR²⁵)₃₋ᵣ](C₁₋₆-alkylene)-, [*—C(OR²⁵)₂O]ₚ(C₁₋₆-alkylene)-, [*—C(R²⁵)(═N—N(R²⁶)C(O)—)]ₚ(C₁₋₆-alkylene)-, [*—C(O)(N(R²⁶)—N═)C(R²⁵)—]ₚ(C₁₋₆-alkylene)-, [*═C(═N—N(R²⁶)C(O)(R²⁵))]ₚ(C₁₋₆-alkylene)-, [*—N(R²⁶)N(R²⁶)]ₚ(C₁₋₆-alkylene)-, [*═C(═N(OH))]ₚ(C₁₋₆-alkylene)-, and [*—OC(R²⁵)(R²⁶)O]ₚ(C₁₋₆-alkylene)-, wherein * represents the attachment point to R⁴; p is 1 or 2; C₁₋₆-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2.
86a. The conjugate of any one of items 82 to 85d and 86, wherein L₁ is selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, [*—SS]ₚ(C₁₋₃-alkylene)-, [*—S(O)₂]ₚ(C₁₋₃-alkylene)-, [(*—O)ᵣC(OR²⁵)₃ . . . ]—(C₁₋₃-alkylene)-, [*—C(OR²⁵)₂O]ₚ(C₁₋₃-alkylene)-, [*—C(R²⁵)(═N—N(R²⁶)C(O)—)]ₚ(C₁₋₃-alkylene)-, [*—C(O)(N(R²⁶)—N═)C(R²⁵)—]ₚ(C₁₋₃-alkylene)-, [*═C(═N—N(R²⁶)C(O)(R²⁵))]ₚ(C₁₋₃-alkylene)-, [*—N(R²⁶)N(R²⁶)]ₚ(C₁₋₃-alkylene)-, [*═C(═N(OH))]ₚ(C₁₋₃-alkylene)-, and [*—OC(R²⁵)(R²⁶)O]ₚ—(C₁₋₃-alkylene)-, wherein * represents the attachment point to R⁴; p is 1 or 2; C₁₋₃-alkylene is either bivalent (if p is 1) or trivalent (if p is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2.
86b. The conjugate of item 86 or 86a, wherein R²⁵ is selected from the group consisting of C₁₋₃ alkyl, phenyl, and phenyl(C₁₋₃ alkyl); and/or R²⁶ is selected from the group consisting of H, C₁₋₃ alkyl, phenyl, and phenyl(C₁₋₃ alkyl).
86c. The conjugate of any one of items 85 to 85d and 86, wherein L₁ is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, preferably from the group consisting of [*—NHC(O)]ₚ(C₁₋₆-alkylene)-, [*—C(0) O]ₚ(C₁₋₆-alkylene)-, [*—OC(O)]ₚ(C₁₋₆-alkylene)-, [*—S]ₚ(C₁₋₆-alkylene)-, and [*—S(O)₂]ₚ(C₁₋₆-alkylene)-, more preferably from the group consisting of [*—NHC(O)]ₚ(C₁₋₆-alkylene)- and [*—C(0) O]ₚ(C₁-6-alkylene)-, wherein * represents the attachment point to R⁴; p is 1 or 2; and C₁₋₆-alkylene is either bivalent (if p is 1) or trivalent (if p is 2).
86d. The conjugate of item 86c, wherein L₁ is selected from the group consisting of [*—NHC(O)]ₚ(C₁₋₃-alkylene)-, [*—C(O)NH]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, and [*—S(O)₂]ₚ(C₁₋₃-alkylene)-, preferably from the group consisting of [*—NHC(O)]ₚ(C₁₋₃-alkylene)-, [*—C(O)O]ₚ(C₁₋₃-alkylene)-, [*—OC(O)]ₚ(C₁₋₃-alkylene)-, [*—S]ₚ(C₁₋₃-alkylene)-, and [*—S(O)₂]ₚ(C₁₋₃-alkylene)-, more preferably from the group consisting of [*—NHC(O)]ₚ(C₁₋₃-alkylene)- and [*—C(O)O]ₚ(C₁₋₃-alkylene)-, wherein * represents the attachment point to R⁴; p is 1 or 2; and C₁₋₃-alkylene is either bivalent (if p is 1) or trivalent (if p is 2).
87. The conjugate of any one of items 82 to 85d and 86 to 86d, wherein L₁ is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R⁴.
88. The conjugate of any one of items 82 to 85d, 86 to 86d, and 87, wherein R² is selected from the group consisting of R⁴, -L₁R⁴, —(CH₂)—CH(OC(O)R⁴)(CH₂OC(O)R⁴), —(CH₂)—CH(SR⁴)₂, and (CH₂)—CH(SR⁴)—CH₂(SR⁴); and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₁ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴.
88a. The conjugate of any one of items 85 to 85d, and 86 to 88, wherein R² is-L₁(R⁴)ₚ.
89. The conjugate of any one of items 82 to 85, 85i, and 85j, wherein L₁ is selected from the group consisting of [*-]₂NC(O)(C₁₋₆-alkylene)-, [*—C(O)O]ₚ(C₁₋₆-alkylene)-OP(O)₂O(C₁₋₆-alkylene) NHC(O)—(C₁₋₆-alkylene)-, [*—OC(O)]ₚ(C₁₋₆-alkylene)-OP(O)₂O(C₁₋₆-alkylene) NHC(O)—(C₁₋₆-alkylene)-, and [*—C(O)]ₚ(C₁₋₆-alkylene)-C(O)—, wherein * represents the attachment point to R⁴; p is 1 or 2; and C₁₋₆-alkylene is either bivalent (if p is 1) or trivalent (if p is 2).
90. The conjugate of any one of items 82 to 85, 85i, 85j, and 89, wherein L₁ is selected from the group consisting of [*-]₂NC(O)(C₁₋₂-alkylene)-, [*—OC(O)]₂ (C₂₋₄-alkylene)-OP(O)₂O(C₂₋₃-alkylene) NHC(O)—(C₁₋₂-alkylene)-, and *—C(O)(C₂₋₃-alkylene)-C(O)—, wherein * represents the attachment point to R⁴.
91. The conjugate of any one of items 82 to 85, 85i, 85j, 89, and 90, wherein R² is selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—.
91a. The conjugate of any one of items 85i, 85j, and 89 to 91, wherein R² is-L₁(R⁴)ₚ.
92. The conjugate of any one of items 82 to 85 and 85e to 85h, wherein the conjugate has the general formula (II′) and L¹ is selected from the group consisting of [*—Z]ₚ(C₁₋₆-alkylene)-Z—, *—Z—(C₃₋₈-cycloalkylene)-Z—, *—Z—(C₃₋₈-cycloalkenylene)-Z—, (*═N)(C₁₋₆-alkylene)-Z—, *—Z—(C₁₋₆-alkylene)-, and *—Z—, wherein * represents the attachment point to R⁴; p is 1 or 2; C₁₋₆-alkylene is either bivalent (if p is 1) or trivalent (if p is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(C₁₋₃-alkylene) NH—, —NH(C₁₋₃-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—.
92a. The conjugate of any one of items 82 to 85, 85e to 85h, and 92, wherein the conjugate has the general formula (II′) and L¹ is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z—(C₁₋₃-alkylene)-, and *—Z—, wherein * represents the attachment point to R⁴; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(C₁₋₂-alkylene) NH—, —NH(C₁₋₂-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²-92b. The conjugate of any one of items 82 to 85, 85e to 85h, 92, and 92a, wherein the conjugate has the general formula (II′) and L¹ is selected from the group consisting of [*—Z]ₚ(C₁₋₃-alkylene)-Z—, *—Z—(C₃₋₆-cycloalkylene)-Z—, *—Z—(C₃₋₆-cycloalkenylene)-Z—, (*═N)(C₁₋₃-alkylene)-Z—, *—Z(C₁₋₃-alkylene)-, and *—Z—, wherein * represents the attachment point to R⁴; p is 1 or 2; C₁₋₃-alkylene is either bivalent (if p is 1) or trivalent (if p is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—.
92c. The conjugate of any one of items 82 to 85, 85e to 85h, 92, 92a, and 92b, wherein the conjugate has the general formula (II′) and L¹ is selected from the group consisting of (*—C(O)O)(CH(OC(O)—*))(CH₂)—Z—, (*═N)(C₁₋₃-alkylene)-NHC(O)—, (*—Z) (C₁₋₃-alkylene)-Z—, *—Z—(C₃₋₆-cycloalkenylene)-Z—, and *—Z—, wherein * represents the attachment point to R⁴; the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
92d. The conjugate of any one of items 82 to 85, 85e to 85h, and 92 to 92c, wherein the conjugate has the general formula (II′) and R² is selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
92e. The conjugate of any one of items 85e to 85h and 92 to 92d, wherein R² is-L₁(R⁴)ₚ.
93. The conjugate of any one of items 82 to 85, wherein the conjugate has the general formula (II′) and L₁ is [*—C(O)]ₚ(C₁₋₆-alkylene)-C(O)NH— or [*-]₂N—, wherein * represents the attachment point to R⁴; p is 1 or 2; and C₁₋₆-alkylene is either bivalent (if p is 1) or trivalent (if p is 2).
94. The conjugate of any one of items 82 to 85 and 93, wherein the conjugate has the general formula (II′) and L₁ is *—C(O)(C₂₋₃-alkylene)-C(O)NH— or [*-]₂N—, wherein * represents the attachment point to R⁴.
95. The conjugate of any one of items 82 to 85, 93, and 94, wherein the conjugate has the general formula (II′) and R² is R⁴—C(O)(CH₂)₂C(O)NH— or R⁴N—.
95a. The conjugate of any one of items 93 to 95, wherein R² is-L₁ (R⁴)ₚ.
96. The conjugate of any one of items 82 to 95a, wherein each R⁴ is independently a non-cyclic, preferably straight hydrocarbyl group.
97. The conjugate of any one of items 82 to 96, wherein each R⁴ is independently a hydrocarbyl group having at least 8 carbon atoms, such as at least 10 carbon atoms.
97a. The conjugate of any one of items 82 to 97, wherein each R⁴ is independently a hydrocarbyl group having at most 16 carbon atoms, such as at most 14 carbon atoms or at most 12 carbon atoms.
97b. The conjugate of any one of items 82 to 97, wherein the conjugate has the general formula (II′) and each R⁴ is independently a hydrocarbyl group having 10 to 18 carbon atoms, such as a straight alkyl group having 10 to 18 carbon atoms or a straight alkenyl group having 10 to 18 carbon atoms.
98. The conjugate of any one of items 82 to 95a and 97, wherein one R⁴ is a tocopherol moiety.
99. The conjugate of any one of items 82 to 98, wherein R³ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.
100. The conjugate of any one of items 82 to 99, wherein R³ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, —C(O)NR²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group.
101. The conjugate of any one of items 82 to 100, wherein R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
102. The conjugate of any one of items 82 to 101, having the following general formula (III) or (III′):

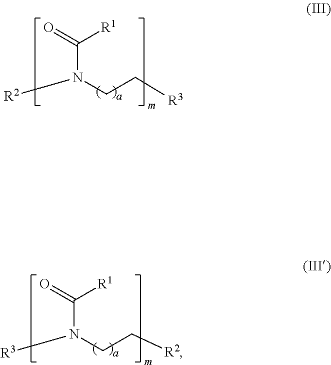

wherein:
a is an integer between 1 and 2;
R¹ is methyl or ethyl and is independently selected for each repeating unit;
m is 10 to 100;
R², for formula (III), is selected from the group consisting of -L₁R⁴, —(CH₂)—CH(OC(O)R⁴)(CH₂OC(O)R⁴), —(CH₂)—CH(SR⁴)₂, and —(CH₂)—CH(SR⁴)—CH₂(SR⁴), wherein each R⁴ is independently a straight hydrocarbyl group having at least 10 carbon atoms; and L₁ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₁ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴; or R², for formula (III′), is selected from the group consisting of (R⁴C(O)O)(CH(OC(O)R⁴))(CH₂)—Z—, (R⁴)₂N(C₁₋₃-alkylene)-Z—, R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z is independently selected the group consisting of —OP(O)₂O(CH₂)₂NH—, from —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
102a. The conjugate of item 102, wherein the conjugate has formula (III) and R² is selected from the group consisting of R⁴—NHC(O)—(CH₂)—, R⁴—NHC(O)—(CH₂)₂—, —(CH₂)—CH(OC(O)R⁴)(CH₂OC(O)R⁴), —(CH₂)—CH(SR⁴)—CH₂(SR⁴), R⁴S—(CH₂)₃—, R⁴S(O)₂—(CH₂)₃—, and R⁴—OC(O)—(CH₂)—.
102b. The conjugate of item 102 or 102a, wherein the conjugate has formula (III) and R³ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.
102c. The conjugate of item 102, wherein the conjugate has formula (III′) and R² is selected from the group consisting of R⁴C(O)NH—, R⁴S—, R⁴S(O)₂—, and R⁴NH-(3,4-dioxocyclobut-1-en-1,2-diyl)-NH—.
102d. The conjugate of item 102 or 102c, wherein the conjugate has formula (III′) and R³ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, preferably R³ is C₁₋₃ alkyl optionally substituted with one substituent selected from the group consisting of —COOH, —COOCH₃, and —C(O)NH(CH₂)₂NH₂.
103. The conjugate of any one of items 82 to 101, having the general formula (III) or (III′) as shown in item 102, wherein:
   a is an integer between 1 and 2;
   R¹ is methyl or ethyl and is independently selected for each repeating unit;
   m is 10 to 100;
   R², for formula (III), is selected from the group consisting of (R⁴)₂NC(O)(C₁₋₂-alkylene)-, R⁴C(O)OCH₂CH(OC(O)R⁴)—CH₂—OP(O)₂O—(CH₂)₂—NHC(O)—CH₂—, and R⁴—C(O)(CH₂)₂—C(O)—, wherein each R⁴ is independently a straight hydrocarbyl group having at least 10 carbon atoms; or R², for formula (III′), is R⁴—C(O)(CH₂)₂C(O)NH— or R⁴N—, wherein each R⁴ is independently a straight hydrocarbyl group having at least 10 carbon atoms or a tocopherol moiety, wherein one R⁴ in the same R² moiety is a tocopherol moiety; and
   R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
104. The conjugate of any one of items 82 to 103, wherein a is 1.
105. The conjugate of any one of items 82 to 103, wherein a is 2.
106. A conjugate of (a) a POX and POZ polymer and (b) one or more hydrophobic chains, wherein the POX and POZ polymer is a copolymer comprising repeating units of the following general formulas (Ia) and (Ib):

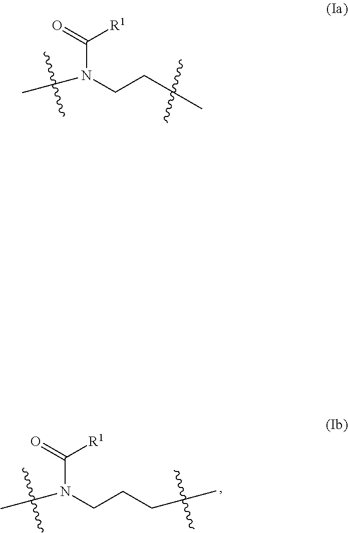

wherein each of R¹ is independently alkyl, in particular C₁₋₃ alkyl, such as methyl, ethyl, iso-propyl, or n-propyl, and is independently selected for each repeating unit; the number of repeating units of formula (Ia) in the copolymer is 1 to 199; the number of repeating units of formula (Ib) in the copolymer is 1 to 199; and the sum of the number of repeating units of formula (Ia) and the number of repeating units of formula (Ib) in the copolymer is 2 to 200.
107. The conjugate of item 106, wherein the conjugate has the following general formula (IV):

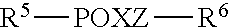

wherein:
R⁵ is R⁷ or -L₁(R⁷)_{q}, wherein each R⁷ is independently a hydrocarbyl group; L₂ is a linker; and
q is 1 or 2;
POXZ is a copolymer containing repeating units of the following general formulas (Ia) and (Ib):

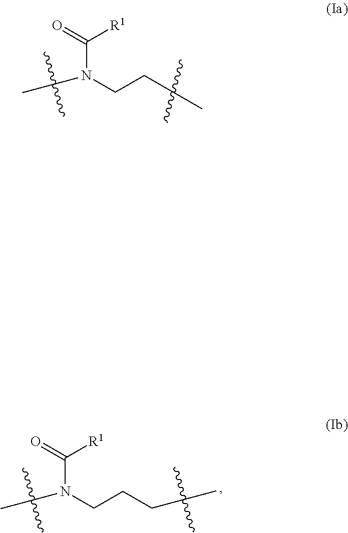

wherein each of R¹ is independently alkyl, in particular C₁₋₃ alkyl, such as methyl, ethyl, iso-propyl, or n-propyl, and is independently selected for each repeating unit; the number of repeating units of formula (Ia) in the copolymer is 1 to 199; the number of repeating units of formula (Ib) in the copolymer is 1 to 199; the sum of the number of repeating units of formula (Ia) and the number of repeating units of formula (Ib) in the copolymer is 2 to 200; and the repeating units of formulas (Ia) and (Ib) are arranged in a random, periodic, alternating or block wise manner; and
R⁶ is selected from the group consisting of H, C₁₋₆ alkyl, C₂₋₆ alkynyl, —OR²⁰, —SR²⁰, halogen, —CN, —N₃, —OC(O)R²¹, —C(O)R²¹, —NR²²R²³, —COOH, —C(O)NR²²R²³, —NR²²C(O)R²¹, a sugar, an amino acid, a peptide, and a member of a targeting pair, wherein the C₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.
107a. The conjugate of item 107, wherein the targeting pair is selected from the following pairs: antigen-antibody specific for said antigen; avidin-streptavidin; folate-folate receptor; transferrin-transferrin receptor; aptamer-molecule for which the aptamer is specific; arginine-glycine-aspartic acid (RGD) peptide-αᵥβ₃ integrin; asparagine-glycine-arginine (NGR) peptide-aminopeptidase N; galactose-asialoglyco-protein receptor.
108. The conjugate of item 106, 107, or 107a, wherein each of R¹ is independently methyl or ethyl, preferably methyl, and is independently selected for each repeating unit.
109. The conjugate of any one of items 106 to 108, wherein the number of repeating units of formula (Ia) in the copolymer is 1 to 179, such as 1 to 159, 1 to 139, 1 to 119 or 1 to 99; the number of repeating units of formula (Ib) in the copolymer is 1 to 179, such as 1 to 159, 1 to 139, 1 to 119 or 1 to 99; and the sum of the number of repeating units of formula (Ia) and the number of repeating units of formula (Ib) in the copolymer is 2 to 180, such as 4 to 160, 6 to 140, 8 to 120 or 10 to 100.
110. The conjugate of any one of items 107 to 109, wherein L₂ comprises at least one functional moiety, such as an alkylene moiety substituted with at least one monovalent functional moiety and/or linked, at the end by which the alkylene group is attached to R⁷, to a divalent functional moiety, wherein preferably each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties.
110a. The conjugate of any one of items 107 to 110, wherein the conjugate comprises one of the following structures and optionally R⁶ is attached to the terminal C atom of the POXZ copolymer:
   (hydrophobic chain)₁₋₂-(alkylene moiety substituted with at least one monovalent functional moiety)-(POXZ copolymer)
   [(hydrophobic chain)-(divalent functional moiety)]₁₋₂-(alkylene moiety)-(POXZ copolymer).
110b. The conjugate of any one of items 107 to 110a, wherein the conjugate has one of the following formulas and optionally R⁶ is attached to the terminal C atom of the POXZ copolymer:
   (hydrophobic chain)₁₋₂-(alkylene moiety substituted with at least one monovalent functional moiety)-(POXZ copolymer)-R⁶
   [(hydrophobic chain)-(divalent functional moiety)]₁₋₂-(alkylene moiety)-(POXZ copolymer)-R⁶.
110c. The conjugate of any one of items 107 to 110b, wherein the alkylene moiety substituted with at least one monovalent functional moiety is substituted with 1, 2, 3, 4, 5, or 6 independently selected monovalent functional moieties.
110d. The conjugate of any one of items 107 to 110c, wherein the alkylene moiety is C₁₋₆-alkylene, such as C₁₋₃-alkylene, e.g., methylene, ethylene, or trimethylene.
110e. The conjugate of any one of items 107 to 110, wherein R⁶ is attached to the terminal N atom of the POXZ copolymer and the conjugate comprises one of the following structures:
   (hydrophobic chain)-(divalent functional moiety)-(POXZ copolymer)
   [(hydrophobic chain)-(divalent functional moiety)]₁₋₂-(alkylene moiety)-(divalent functional moiety)-(POXZ copolymer)
   (hydrophobic chain)-(divalent functional moiety)-(cycloalkylene moiety)-(divalent functional moiety)-(POXZ copolymer)
   (hydrophobic chain)-(divalent functional moiety)-(cycloalkenylene moiety)-(divalent functional moiety)-(POXZ copolymer)
   (hydrophobic chain)-(divalent functional moiety)-(alkylene moiety)-(POXZ copolymer)
   [(hydrophobic chain)₂-(trivalent functional moiety)]-(alkylene moiety)-(divalent functional moiety)-(POXZ copolymer)
110f. The conjugate of any one of items 107 to 110 and 110e, wherein R⁶ is attached to the terminal N atom of the POXZ copolymer and the conjugate has one of the following formulas:
   (hydrophobic chain)-(divalent functional moiety)-(POXZ copolymer)-R⁶
   [(hydrophobic chain)-(divalent functional moiety)]₁₋₂-(alkylene moiety)-(divalent functional moiety)-(POXZ copolymer)-R⁶
   (hydrophobic chain)-(divalent functional moiety)-(cycloalkylene moiety)-(divalent functional moiety)-(POXZ copolymer)-R⁶
   (hydrophobic chain)-(divalent functional moiety)-(cycloalkenylene moiety)-(divalent functional moiety)-(POXZ copolymer)-R⁶
   (hydrophobic chain)-(divalent functional moiety)-(alkylene moiety)-(POXZ copolymer)-R⁶
   [(hydrophobic chain)₂-(trivalent functional moiety)]-(alkylene moiety)-(divalent functional moiety)-(POXZ copolymer)-R⁶
110g. The conjugate of item 110e or 110f, wherein the alkylene moiety is C₁₋₆-alkylene, such as C₁₋₃-alkylene, e.g., methylene, ethylene, or trimethylene.
110h. The conjugate of any one of items 110e to 110g, wherein the cycloalkylene moiety is C₃₋₈-cycloalkylene, such as C₃₋₆-cycloalkylene, e.g., cyclopropylene, cyclobutylene, cyclopentylene, cyclohexylene; and/or the cycloalkenylene moiety is C₃₋₈-cycloalkenylene, such as C₃₋₆-cycloalkenylene, e.g., cyclopropenylene, cyclobutenylene, cyclopentenylene, cyclohexenylene, wherein each of the cycloalkylene and cycloalkenylene moieties is optionally substituted with one or more (e.g., 1, 2, 3, or 4) substituents (e.g., independently selected from the group consisting of —OH, ═O, —SH, halogen, —CN, —N₃, and C₁₋₃-alkyl).
110i. The conjugate of any one of any one of items 107 to 110, wherein the conjugate comprises the following structure and optionally R⁶ is attached to the terminal C atom of the POXZ copolymer:
   [(hydrophobic chain)₂-(trivalent functional moiety)]-(alkylene moiety)-(POX and/or POZ polymer).
110j. The conjugate of any one of any one of items 107 to 110 and 110i, wherein the conjugate has the following formula and optionally R⁶ is attached to the terminal C atom of the POXZ copolymer:
   [(hydrophobic chain)₂-(trivalent functional moiety)]-(alkylene moiety)-(POX and/or POZ polymer)-R⁶.
111. The conjugate of any one of items 107 to 110d, wherein L₂ is selected from the group consisting of [*—NHC(O)], (C₁₋₆-alkylene)-, [*—C(O)NH], (C₁₋₆-alkylene)-, [*—C(O)O], (C₁₋₆-alkylene)-, [*—OC(O)], (C₁₋₆-alkylene)-, [*—S]_{q}(C₁₋₆-alkylene)-, [*—SS]_{q}(C₁₋₆-alkylene)-, [*—S(O)₂]ₚ(C₁₋₆-alkylene)-, [(*—O)ᵣC(OR²⁵)₃₋ₛ](C₁₋₆-alkylene)-, [*—C(OR²⁵)₂O], (C₁₋₆-alkylene)-, [*—C(R²⁵)(═N—N(R²⁶)C(O)—)]_{q}—(C₁₋₆-alkylene)-, [*—C(O)(N(R²⁶)—N═)C(R²⁵)—]_{q}(C₁₋₆-alkylene)-, [*═C(═N—N(R²⁶)C(O)(R²⁵))]_{q}(C₁₋₆-alkylene)-, [*—N(R²⁶)N(R²⁶)], (C₁₋₆-alkylene)-, [*═C(═N(OH))], (C₁₋₆-alkylene)-, and [*—OC(R²⁵)(R²⁶)O], (C₁₋₆-alkylene)-, wherein * represents the attachment point to R⁷; q is 1 or 2; C₁₋₆-alkylene is either bivalent (if q is 1) or trivalent (if q is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and s is an integer between 1 and 2.
111a. The conjugate of any one of items 107 to 110d and 111, wherein L₂ is selected from the group consisting of [*—NHC(O)], (C₁₋₃-alkylene)-, [*—C(O)NH]_{q}(C₁₋₃-alkylene)-, [*—C(O)O]_{q}(C₁₋₃-alkylene)-, [*—OC(O)], (C₁₋₃-alkylene)-, [*—S]_{q}(C₁₋₃-alkylene)-, [*—SS], (C₁₋₃-alkylene)-, [*—S(O)₂]ₚ(C₁₋₃-alkylene)-, [(*—O)ₛC(OR²⁵)₃₋ₛ]—(C₁₋₃-alkylene)-, [*—C(OR²⁵)₂O], (C₁₋₃-alkylene)-, [*—C(R²⁵)(═N—N(R²⁶)C(O)—)], (C₁₋₃-alkylene)-, [*—C(O)(N(R²⁶)—N═)C(R²⁵)—]_{q}(C₁₋₃-alkylene)-, [*═C(═N—N(R²⁶)C(O)(R²⁵))]_{q}(C₁₋₃-alkylene)-, [*—N(R²⁶)N(R²⁶)], (C₁₋₃-alkylene)-, [*═C(═N(OH))], (C₁₋₃-alkylene)-, and [*—OC(R²⁵)(R²⁶)O]_{q}—(C₁₋₃-alkylene)-, wherein * represents the attachment point to R⁷; q is 1 or 2; C₁₋₃-alkylene is either bivalent (if q is 1) or trivalent (if q is 2); R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and s is an integer between 1 and 2.
111b. The conjugate of item 111 or 111a, wherein R²⁵ is selected from the group consisting of C₁₋₃ alkyl, phenyl, and phenyl(C₁₋₃ alkyl); and/or R²⁶ is selected from the group consisting of H, C₁₋₃ alkyl, phenyl, and phenyl(C₁₋₃ alkyl).
111c. The conjugate of any one of items 107 to 110d and 111, wherein L₂ is selected from the group consisting of [*—NHC(O)], (C₁₋₆-alkylene)-, [*—C(O)NH], (C₁₋₆-alkylene)-, [*—C(O)O]_{q}(C₁₋₆-alkylene)-, [*—OC(O)], (C₁₋₆-alkylene)-, [*—S], (C₁₋₆-alkylene)-, and [*—S(O)₂]ₚ(C₁₋₆-alkylene)-, preferably from the group consisting of [*—NHC(O)]_{q}(C₁₋₆-alkylene)-, [*—C(O)O], (C₁₋₆-alkylene)-, [*—OC(O)], (C₁₋₆-alkylene)-, [*—S], (C₁₋₆-alkylene)-, and [*—S(O)₂]ₚ(C₁₋₆-alkylene)-, more preferably from the group consisting of [*—NHC(O)], (C₁₋₆-alkylene)- and [*—C(O)O], (C₁₋₆-alkylene)-, wherein * represents the attachment point to R⁷; q is 1 or 2; and C₁₋₆-alkylene is either bivalent (if q is 1) or trivalent (if q is 2).
111d. The conjugate of item 111c, wherein L₂ is selected from the group consisting of [*—NHC(O)], (C₁₋₃-alkylene)-, [*—C(O)NH]_{q}(C₁₋₃-alkylene)-, [*—C(O)O], (C₁₋₃-alkylene)-, [*—OC(O)]_{q}(C₁₋₃-alkylene)-, [*—S], (C₁₋₃-alkylene)-, and [*—S(O)₂]ₚ(C₁₋₃-alkylene)-, preferably from the group consisting of [*—NHC(O)]_{q}(C₁₋₃-alkylene)-, [*—C(O)O]_{q}(C₁₋₃-alkylene)-, [*—OC(O)]_{q}(C₁₋₃-alkylene)-, [*—S], (C₁₋₃-alkylene)-, and [*—S(O)₂]ₚ(C₁₋₃-alkylene)-, more preferably from the group consisting of [*—NHC(O)], (C₁₋₃-alkylene)- and [*—C(O)O]_{q}(C₁₋₃-alkylene)-, wherein * represents the attachment point to R⁷; q is 1 or 2; and C₁₋₃-alkylene is either bivalent (if q is 1) or trivalent (if q is 2).
112. The conjugate of any one of items 107 to 110d and 111 to 111d, wherein L₂ is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), —(CH₂)—CH(S—*)₂, —(CH₂)—CH(S—*)—CH₂(S—*), *—S—(CH₂)₃—, *—S(O)₂—(CH₂)₃—, and *—OC(O)—(CH₂)—, wherein * represents the attachment point to R⁷.
113. The conjugate of any one of items 107 to 110d, 111 to 111d, and 112, wherein R⁵ is selected from the group consisting of R⁷, -L₁R⁷, —(CH₂)—CH(OC(O)R⁷)(CH₂OC(O)R⁷), —(CH₂)—CH(SR⁷)₂, and (CH₂)—CH(SR⁷)—CH₂(SR⁷); and L₂ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₂ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁷.
113a. The conjugate of any one of items 107 to 110d and 111 to 113, wherein R⁵ is-L₁(R⁷)_{q}.
114. The conjugate of any one of items 107 to 110, 110i, and 110j, wherein L₂ is selected from the group consisting of [*-]₂NC(O)(C₁₋₆-alkylene)-, [*—C(O)O], (C₁₋₆-alkylene)-OP(O)₂O(C₁₋₆-alkylene) NHC(O)—(C₁₋₆-alkylene)-, [*—OC(O)]_{q}(C₁₋₆-alkylene)-OP(O)₂O(C₁₋₆-alkylene) NHC(O)—(C₁₋₆-alkylene)-, and [*—C(O)]_{q}(C₁₋₆-alkylene)-C(O)—, wherein * represents the attachment point to R⁷; q is 1 or 2; and C₁₋₆-alkylene is either bivalent (if q is 1) or trivalent (if q is 2).
115. The conjugate of any one of items 107 to 110, 110i, 110j, and 114, wherein L₂ is selected from the group consisting of [*-]₂NC(O)(C₁₋₂-alkylene)-, [*—OC(O)]₂ (C₂₋₄-alkylene)-OP(O)₂O(C₂₋₃-alkylene) NHC(O)—(C₁₋₂-alkylene)-, and *—C(O)(C₂₋₃-alkylene)-C(O)—, wherein * represents the attachment point to R⁷.
116. The conjugate of any one of items 107 to 110, 110i, 110j, 114, and 115, wherein R⁵ is selected from the group consisting of (R⁷), NC(O)(C₁₋₂-alkylene)-, R⁴C(O)OCH₂CH(OC(O)R⁷)—CH₂—OP(O)₂O—(CH₂)₂—NHC(O)—CH₂—, and R⁷—C(O)(CH₂)₂—C(O)—.
116a. The conjugate of any one of items 110i, 110j, and 114 to 116, wherein R⁵ is-L₂ (R⁷)_{q}.
117. The conjugate of any one of items 107 to 110 and 110e to 110h, wherein R⁶ is attached to the N-end of the POXZ copolymer; and L₂ is attached to the C-end of the POXZ copolymer and is selected from the group consisting of [*—Z¹], (C₁₋₆-alkylene)-Z¹—, *—Z¹—(C₃₋₈-cycloalkylene)-Z¹—, *—Z¹—(C₃₋₈-cycloalkenylene)-Z¹—, (*═N)(C₁₋₆-alkylene)-Z¹—, *—Z¹ (C₁₋₆-alkylene)-, and *—Z¹—, wherein * represents the attachment point to R⁷; q is 1 or 2; C₁₋₆-alkylene is either bivalent (if q is 1) or trivalent (if q is 2); each of the C₃₋₈-cycloalkylene and C₃₋₈-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(C₁₋₃-alkylene) NH—, —NH(C₁₋₃-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—.
117a. The conjugate of any one of items 107 to 110, 110e to 110h, and 117, wherein R⁶ is attached to the N-end of the POXZ copolymer; and L₁ is attached to the C-end of the POXZ copolymer and is selected from the group consisting of [*—Z¹], (C₁₋₃-alkylene)-Z¹—, *—Z¹—(C₃₋₆-cycloalkylene)-Z¹—, *—Z¹—(C₃₋₆-cycloalkenylene)-Z¹—, (*═N)(C₁₋₃-alkylene)-Z¹—, *—Z¹ (C₁₋₃-alkylene)-, and *—Z¹—, wherein * represents the attachment point to R⁷; q is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(C₁₋₂-alkylene) NH—, —NH(C₁₋₂-alkylene) OP(O)₂O—, —C(O)NH—, —NHC(O)—, —OC(O)NH—, —NHC(O)O—, —O—, —C(O)O—, —OC(O)—, —S—, —S(O)₂—, and —NR²²—.
117b. The conjugate of any one of items 107 to 110, 110e to 110h, 117, and 117a, wherein R⁶ is attached to the N-end of the POXZ copolymer; and L₂ is attached to the C-end of the POXZ copolymer and is selected from the group consisting of [*—Z¹], (C₁₋₃-alkylene)-Z¹—, *—Z¹—(C₃₋₆-cycloalkylene)-Z¹—, *—Z¹—(C₃₋₆-cycloalkenylene)-Z¹—, (*═N)(C₁₋₃-alkylene)-Z¹—, *—Z¹ (C₁₋₃-alkylene)-, and *—Z¹—, wherein * represents the attachment point to R⁷; p is 1 or 2; C₁₋₃-alkylene is either bivalent (if q is 1) or trivalent (if q is 2); each of the C₃₋₆-cycloalkylene and C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, —N(C₁₋₃-alkyl)-, and —NH—.
117c. The conjugate of any one of items 107 to 110, 110e to 110h, 117, 117a, and 117b, wherein R⁶ is attached to the N-end of the POXZ copolymer; and L₂ is attached to the C-end of the POXZ copolymer and is selected from the group consisting of (*—C(O)O)(CH(OC(O)—*))(CH₂)—Z¹—, (*═N)(C₁₋₃-alkylene)-NHC(O)—, (*—Z¹ (C₁₋₃-alkylene)-Z¹—, *—Z¹—(C₃₋₆-cycloalkenylene)-Z¹—, and *—Z¹—, wherein * represents the attachment point to R⁷; the C₃₋₆-cycloalkenylene groups is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
117d. The conjugate of any one of items 107 to 110, 110e to 110h, and 117 to 117c, wherein R⁶ is attached to the N-end of the POXZ copolymer; and R⁵ is attached to the C-end of the POXZ copolymer and is selected from the group consisting of (R⁴C(O)O)(CH(OC(O)R⁷))(CH₂)—Z¹—, (R⁷)₂N(C₁₋₃-alkylene)-Z¹—, R⁷Z¹ (C₁₋₃-alkylene)-Z¹—, R⁷Z¹—(C₃₋₆-cycloalkenylene)-Z¹—, and R⁷Z¹—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—.
117e. The conjugate of any one of items 110e to 110h and 117 to 117d, wherein R⁵ is-L₂ (R⁷)_{q}.
117f. The conjugate of any one of items 107 to 117, wherein R⁵ is-L₂ (R⁷)_{q}.
118. The conjugate of any one of items 107 to 110, wherein R⁶ is attached to the N-end of the POXZ copolymer; and L₂ is attached to the C-end of the POXZ copolymer and is selected from the group consisting of [*—C(O)]_{q}(C₁₋₆-alkylene)-C(O)NH— and [*-]₂N—, wherein * represents the attachment point to R⁷; q is 1 or 2; and C₁₋₆-alkylene is either bivalent (if q is 1) or trivalent (if q is 2).
119. The conjugate of any one of items 107 to 110 and 118, wherein R⁶ is attached to the N-end of the POXZ copolymer; and L₂ is attached to the C-end of the POXZ copolymer and is *—C(O)(C₂₋₃-alkylene)-C(O)NH— or [*-]₂N—, wherein * represents the attachment point to R⁷.
120. The conjugate of any one of items 107 to 110, 118, and 119, wherein R⁶ is attached to the N-end of the POXZ copolymer; and R⁵ is attached to the C-end of the POXZ copolymer and is R⁷—C(O)(CH₂)₂C(O)NH— or R⁷N—.
120a. The conjugate of any one of items 118 to 120, wherein R⁵ is-L₂ (R⁷)_{q}.
121. The conjugate of any one of items 107 to 120a, wherein each R⁷ is independently a non-cyclic, preferably straight hydrocarbyl group.
122. The conjugate of any one of items 107 to 121, wherein each R⁷ is independently a hydrocarbyl group having at least 8 carbon atoms, such as at least 10 carbon atoms.
122a. The conjugate of any one of items 107 to 122, wherein each R⁷ is independently a hydrocarbyl group having at most 16 carbon atoms, such as at most 14 carbon atoms or at most 12 carbon atoms.
122b. The conjugate of any one of items 107 to 122, wherein R⁵ is attached to the C-end of the POXZ copolymer and each R⁷ is independently a hydrocarbyl group having 10 to 18 carbon atoms, such as a straight alkyl group having 10 to 18 carbon atoms or a straight alkenyl group having 10 to 18 carbon atoms.
123. The conjugate of any one of items 107 to 120a and 122, wherein one R⁷ is a tocopherol moiety.
124. The conjugate of any one of items 107 to 123, wherein R⁶ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair.
125. The conjugate of any one of items 107 to 124, wherein R⁶ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, —C(O)NR²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group.
126. The conjugate of any one of items 107 to 125, wherein R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
127. The conjugate of any one of items 106 to 126, having the following general formula (V):

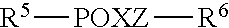

wherein:
R⁵, when attached to the N-end of the POXZ copolymer, is selected from the group consisting of -L₂R⁷, —(CH₂)—CH(OC(O)R⁷)(CH₂OC(O)R⁷), —(CH₂)—CH(SR⁷)₂, and —(CH₂)—CH(SR⁷)—CH₂(SR⁷), wherein each R⁷ is independently a straight hydrocarbyl group having at least 10 carbon atoms; and L₂ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂)— (preferably L₂ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁷, or R⁵, when attached to the C-end of the POXZ copolymer, is selected from the group consisting of (R⁷C(O)O)(CH(OC(O)R⁷))(CH₂)—Z¹—, (R⁷)₂N(C₁₋₃-alkylene)-Z¹—, R⁷Z¹(C₁₋₃-alkylene)-Z¹—, R⁷Z¹—(C₃₋₆-cycloalkenylene)-Z¹—, and R⁷Z¹—, wherein the C₃₋₆-cycloalkenylene group is optionally substituted with one or more (e.g., 1, 2, 3, or 4) substituents independently selected from the group consisting of —OH, ═O, —SH, halogen, —CN, —N₃, and C₁₋₃-alkyl; and each Z¹ is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, —OC(O)—, —S—, —S(O)₂—, and —NH—;
POXZ is a copolymer containing the repeating units of the following general formulas (Ia) and (Ib):

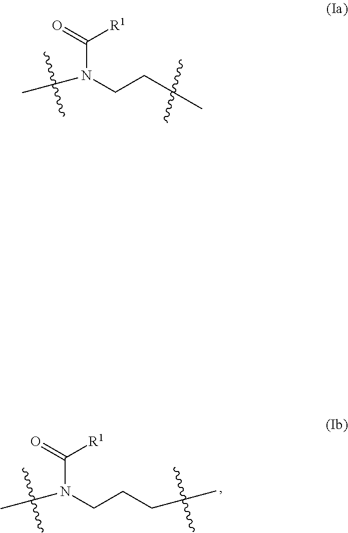

wherein each of R¹ is independently methyl or ethyl and is independently selected for each repeating unit; the number of repeating units of formula (Ia) in the copolymer is 1 to 99; the number of repeating units of formula (Ib) in the copolymer is 1 to 99; the sum of the number of repeating units of formula (Ia) and the number of repeating units of formula (Ib) in the copolymer is 10 to 100; and the repeating units of formulas (Ia) and (Ib) are arranged in a random, periodic, alternating or block wise manner; and
R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
127a. The conjugate of item 127, wherein R⁵ is attached to the N-end of the POXZ copolymer and is selected from the group consisting of R⁷—NHC(O)—(CH₂)—, R⁷—NHC(O)—(CH₂)₂—, —(CH₂)—CH(OC(O)R⁷)(CHOC(O)R⁷), —(CH₂)—CH(SR⁷)—CH₂(SR⁷), R'S—(CH₂)₃—, R'S(O)₂—(CH₂)₃—, and R⁷—OC(O)—(CH₂)—.
127b. The conjugate of item 127 or 127a, wherein R⁵ is attached to the N-end of the POXZ copolymer; and R⁶ is selected from the group consisting of —OH, —N₃, C₂₋₆ alkynyl, —COOH, —NH₂, —NHCH₃, —N(CH₃)₂, —NHC(O)(CH₂)₂COOH, —N(CH₂CH₃)C(O)(CH₂)₂COOH, —N(CH₂CH₃)C(O) CH₃, —NH(CH₂CH₃), —OC(O)(CH₂)₂COOH, and a member of a targeting pair, such as from the group consisting of —OH, —N₃, —NH₂, —NHC(O)(CH₂)₂COOH, —N(CH₂CH₃)C(O)(CH₂)₂COOH, —N(CH₂CH₃)C(O) CH₃, —NH(CH₂CH₃), and —OC(O)(CH₂)₂COOH.
127c. The conjugate of item 127, wherein R⁵ is attached to the C-end of the POXZ copolymer and is selected from the group consisting of RIC(O)NH—, R⁷S—, R⁷S(O)₂—, and R⁷NH-(3,4-dioxocyclobut-1-en-1,2-diyl)-NH—.
127d. The conjugate of item 127 or 127c, wherein R⁵ is attached to the C-end of the POXZ copolymer; and R⁶ is C₁₋₃ alkyl optionally substituted with one or two substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair, preferably R⁶ is C₁₋₃ alkyl optionally substituted with one substituent selected from the group consisting of —COOH, —COOCH₃, and —C(O)NH(CH₂)₂NH₂.
128. The conjugate of any one of items 106 to 126, having the general formula (V) as shown in item 127, wherein:
   R⁵, when attached to the N-end of the POXZ copolymer, is selected from the group consisting of (R⁷)₂NC(O)(C₁₋₂-alkylene)-, R⁷C(O)OCH₂CH(OC(O)R⁷)—CH₂—OP(O)₂O—(CH₂)₂—NHC(O)—CH₂—, and R⁷—C(O)(CH₂)₂—C(O)—, wherein each R⁷ is independently a straight hydrocarbyl group having at least 10 carbon atoms, or R⁵, when attached to the C-end of the POXZ copolymer, is R⁷—C(O)(CH₂)₂C(O)NH— or R⁷N—, wherein each R⁷ is independently a straight hydrocarbyl group having at least 10 carbon atoms or a tocopherol moiety, wherein one R⁷ in the same R⁵ moiety is a tocopherol moiety;
   POXZ is a copolymer containing the repeating units of the general formulas (Ia) and (Ib) as shown in item 127, wherein each of R¹ is independently methyl or ethyl and is independently selected for each repeating unit; the number of repeating units of formula (Ia) in the copolymer is 1 to 99; the number of repeating units of formula (Ib) in the copolymer is 1 to 99; the sum of the number of repeating units of formula (Ia) and the number of repeating units of formula (Ib) in the copolymer is 10 to 100; and the repeating units of formulas (Ia) and (Ib) are arranged in a random, periodic, alternating or block wise manner; and
   R⁶ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair.
129. A composition comprising:
   (i) nucleic acid;
   (ii) a cationic or cationically ionizable lipid; and
   (iii) a conjugate of any one of items 82 to 128.
130. A method for delivering nucleic acid to cells of a subject, the method comprising administering to a subject a composition of item 129.
131. A method for delivering a therapeutic peptide or protein to a subject, the method comprising administering to a subject a composition of item 129, wherein the nucleic acid encodes the therapeutic peptide or protein.
132. A method for treating or preventing a disease or disorder in a subject, the method comprising administering to a subject a composition of item 129, wherein delivering the nucleic acid to cells of the subject is beneficial in treating or preventing the disease or disorder.
133. A method for treating or preventing a disease or disorder in a subject, the method comprising administering to a subject a composition of item 129, wherein the nucleic acid encodes a therapeutic peptide or protein and wherein delivering the therapeutic peptide or protein to the subject is beneficial in treating or preventing the disease or disorder.
134. The method of any one of items 130 to 133, wherein the subject is a mammal.
135. The method of item 134, wherein the mammal is a human.

Further aspects of the present disclosure are disclosed herein.

### EXAMPLES

### Materials and Methods

Preparation of Conjugates of (a) a Polyoxazoline (POX) and/or Polyoxazine (POZ) Polymer and (b) One or More Hydrophobic Chains

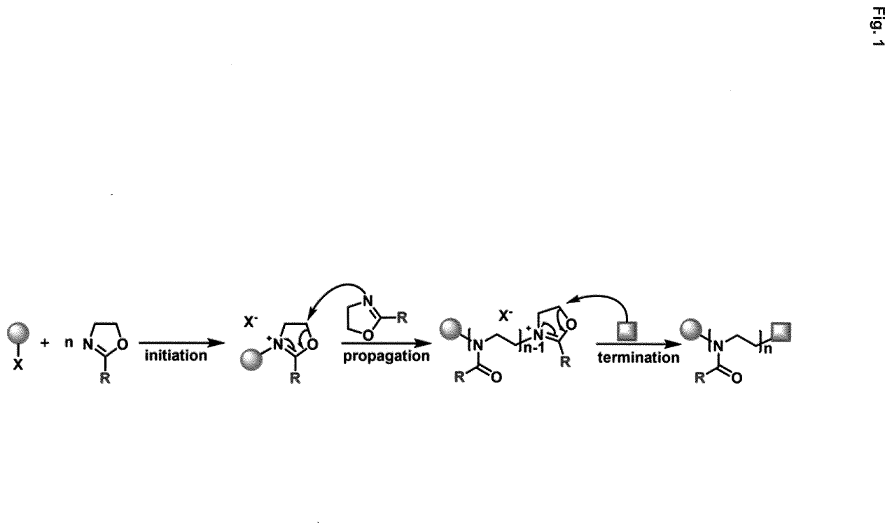

A scheme for preparing conjugates of (a) a polyoxazoline (POX) polymer and (b) one or more hydrophobic chains by living polymerization is depicted in FIG. 1. However, the same protocol can be utilized for preparing conjugates of (a) a polyoxazine (POZ) polymer and (b) more than a hydrophobic chain or for conjugates of (a) a POXZ copolymer and (b) one or more hydrophobic chains.

Briefly, freshly distilled monomer (e.g., 2-methyl-4,5-dihydro-1,3-oxazole), solvent (e.g., dry acetonitrile), and initiator (e.g., ethyl 3-bromopropionate) is added to a screw-cap tube under nitrogen purge, the tube is sealed, and left stirring in an oil bath at 80° C. for 18 h. The amount of initiator (mole ratio of initiator to monomer) is determined by the desired degree of polymerization. Termination can be performed by adding a methanolic solution of KOH (0.5 M) and stirring at 25° C. for 2 h which causes both conversion of the terminal oxazolinium ion into an N-(2-hydroxyethyl) group and conversion of the ethyl ester group of the initiator into a carboxyl group. Treatment with an amine containing one or more hydrophobic chains (such as myristyl amine), optionally in the presence of coupling additives (such as N-hydroxysuccinimide and/or dicyclohexylcarbodiimide), results in the desired conjugate.

### Preparation of LNPs

A protocol for preparing an RNA LNP formulation is described here utilizing specific components (i.e., modified mRNA coding for luciferase as an example for RNA; DODMA as an example for the cationically ionizable lipid; C14-PMeOx₄₅₋₅₀-OH as an example for the conjugate of (a) a polyoxazoline (POX) and/or polyoxazine (POZ) polymer and (b) one or more hydrophobic chains; DOPE as an example of a phospholipid; and cholesterol as an example of a steroid). However, the same protocol applies as well for other components (e.g., other RNAs (e.g., mRNA coding for a vaccine antigen); other cationically ionizable lipids (e.g., DOTAP); other conjugates of (a) a polyoxazoline (POX) and/or polyoxazine (POZ) polymer and (b) one or more hydrophobic chains (e.g., C14-NHCO—PMeOx₄₅₋₅₀-OH); and/or other additional lipids (e.g., other phospholipids such as DSPC) and/or other steroids).

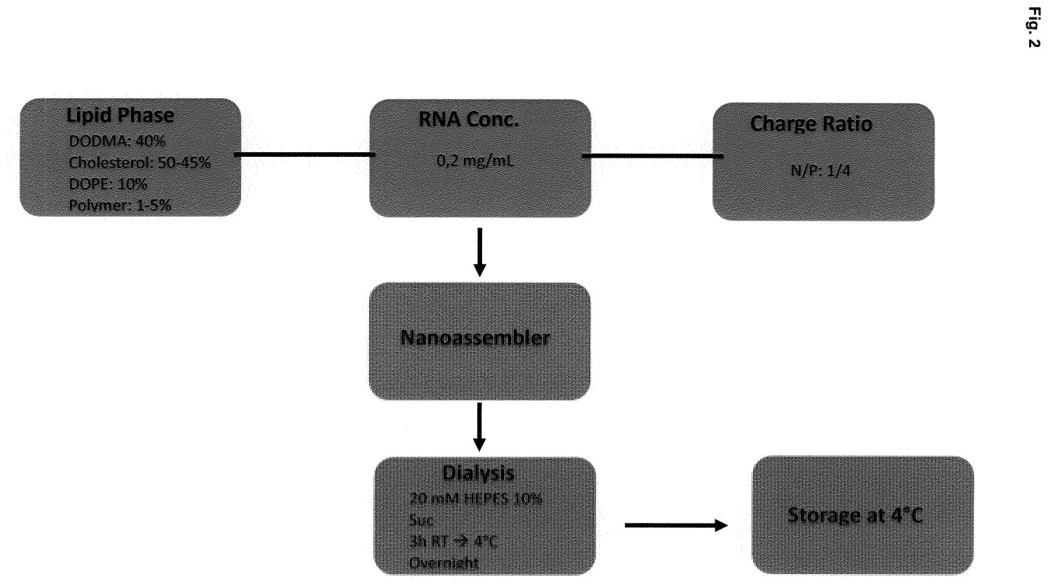

RNA lipid nanoparticles were prepared by an aqueous-ethanol mixing protocol. Briefly, an aqueous mRNA solution (comprising 100 mM citrate buffer at pH 4.0 and 0.2 mg/mL mRNA coding for luciferase) was mixed with an ethanolic lipid mix (comprising DODMA, cholesterol, DOPE, and C14-PMeOx₄₅₋₅₀-OH in molar ratio of 40:48-Y: 10:Y, respectively) in a volume ratio of 3 parts of RNA solution to 1 part of ethanolic lipid mix. The mixing was achieved using a microfluidic system (Nanoassembler). After concentration and exchange of the buffer (to 20 mM HEPES, pH 6, 10% sucrose), the RNA LNP formulation (0.1 mg/ml) was obtained which is stored at 4° C. until use; cf., also FIG. 2.

Alternatively, lipid nanoparticles were prepared by mixing an aqueous phase (0.15 mg/mL mRNA diluted in 0.1 M citrate buffer or in 0.05 M acetate buffer, pH 4.0) and an organic phase (a lipid mixture of cationic/cationically ionizable lipid:DSPC:cholesterol:stealth component dissolved in ethanol at a molar fraction of 47.5:42.5-X: 10:X, respectively, with N/P=6 and a total concentration of 17.05 mM) at a 3:1 volume ratio and 12 mL/min, using a microfluidic instrument (NanoAssemblr® Benchtop, Precision NanoSystems, Vancouver, Canada). The mixture was dialyzed against 1×DPBS (GIBCO, pH 7.4) or HEPES 20 Mm (GIBCO, pH 6.0) for 3 h in a Slide-A-Lyser 10K MWCO dialysis cassette (Thermo Fisher Scientific, Waltham, MA, USA.) In these experiments, residual ethanol was regularly controlled by osmolality measurements. The physicochemical characterization (size, polydispersity, zeta potential, RNA accessibility and total RNA concentration) was performed on the day of preparation. After complete characterization, formulations were stored at 4° C. for not more than 1 day or at −80° C. Lipid nanoparticles were diluted in PBS to the desired RNA concentration prior to in vitro testing or in vivo injection.

### In-Vitro Luciferase Expression

modRNA expression was measured after 24 hrs of transfection with RNA LNP formulations. The formulations were diluted to 50 ng of formulation buffer. 96-well plates were seeded with 5×10³ C2C12 or 2.5×10⁴ RAW or 2.0×10⁴ HepG2 cells/well. After 24 h, the medium was aspirated and 90 μL of the culture medium with 10 μL of the diluted formulation was added. The plates were further kept in the cell incubator for 24 h. Bright-glow kit was used to measure luciferase expression in each well. The manufacturer's protocols were strictly followed. Luminescence measurements were performed in Tecan Infinite Pro200 in white bottom plates.

### Size Measurements

Size measurements were performed with a DynaPro Plate Reader II (Wyatt, Dernbach, Germany). The measurement principle is based on dynamic light scattering and calculates the hydrodynamic diameter of the nanoparticles. The formulated LNPs and liposomes were diluted to 0.0125 mg/mL with the dialysis buffer or to 0.005 mg/mL with 1×PBS to a total volume of 120 μL and then measured in triplicates in 96 well plates. 10 exposures per well were performed, each lasting 10 seconds. From the measurements, size (Zaverage), and polydispersity indices (PdI) were calculated from the cumulant analysis using Dynamics 7.8.1.3 software.

### Zeta-Potential

The electrophoretic mobility of the LNP emulsion was measured using a WALLIS™ Zeta Potential Analyzer. The following instrument settings were selected: 25° C., viscosity: 1.13 n, ten individual measurements per sequence with medium high resolution and a measurement duration of 7.5 min. The electrophoretic mobility of the colloids was determined using the Henry function according to Smulochowski. For the measurement, the measuring electrode was first rinsed with 96% ethanol and then washed with deionized water. Filtered compressed air was used for drying. The samples were measured in their respective storage buffers. Low salt buffers were used to keep the ion concentration below 15 mM. 100 μL of 0.1 μg/uL sample concentration was placed in a cuvette and then diluted with 1000 μL of 20 mM HEPES 10% sucrose. The electrode was then immersed in the cuvette free of air bubbles. After insertion into the designated apparatus, measurements were performed using the above parameters.

Alternatively, the zeta potential of particles was determined by photon correlation spectroscopy using particles sizer (zeta potential/particle sizer, Nicomp™ 380 ZLS, Santa Barbara, CA, USA) with an E-field strength of 4 V/cm and an electrode spacing of 0.4 cm in a plastic cuvette. LNP formulations were diluted 1:26 in 0.1×PBS for zeta potential measurement. The electrostatic mobility was converted to the zeta potential using the Helmholtz-Smoluchowski equation. All measurements were carried out at a temperature of 23° C.

### RNA-Accessibility

To determine the non-accessible RNA, a RiboGreen assay was performed. Free RNA was fluorescently labeled with an intercalating dye (RiboGreen) and quantified using a Tecan™ microplate reader. The LNP emulsion was diluted in a 96-well plate to a concentration of 0.002 mg/mL with TE buffer (pH=6.0). The total volume per well was 300 μL. From each of these dilutions, 50 L was then taken and mixed with another 50 μL of TE buffer for the measurements in the native state. For measurements in the denatured state, 50 μL of the dilutions were mixed with 50 μL of 2% Triton-TE buffer. To determine the total RNA concentration, a standard curve was prepared. For this purpose, the RNA was diluted to a final concentration of 20 μg/mL with TE buffer. A dilution series was then prepared with the following concentrations: 1.5, 1.0, 0.5, 0.25, 0.1 μg/mL. All wells were pipetted in duplicates. The plate was then incubated at 40° C. for 7 min. Afterwards, 50 L of the fluorophore RiboGreen previously diluted 1:100 with TE buffer was added. For fluorescence measurement, Tecan™ Reader was used with the following parameters: Shaking 5 sec with amplitude 2.5, Excitation 480, Emission 525, 25 flashes. the RNA concentration is then calculated from the data.

In an alternative approach, a modified Quant-iT RiboGreen RNA assay (Invitrogen, Carlsbad, CA) was used. LNP samples were diluted in 1×TE buffer pH 7.4 to a mRNA concentration between 2 and 5 ng/μL. Accessible mRNA was measured by diluting the sample in 1×TE, and the total RNA amount was quantified by diluting the sample in 2% Triton X-100 (VWR International GmbH, Darmstadt, Germany). Ribogreen reagent was added to each sample, and the fluorescent signal was quantified in an Infinite F200PRO microplate reader (Tecan, Männedorf, Switzerland). For the determination of total RNA concentration, a standard curve of Ribogreen fluorescence versus mRNA was used between 0-2.5 μg/mL mRNA in 1% Triton X-100 (pH 7.4).

### Agarose Gel Electrophoresis

Agarose gel electrophoresis was performed to evaluate free RNA. The gel was poured by using 1 g agarose dissolved in 100 mL of 1×TAE Buffer pH 7.4 (Tris-acetate-EDTA) (Rotiphorese® 50×TAE, Carl Roth, Karlsruhe, Germany), 1 mL of 5% Sodium hypochlorite, and 10 μL of GelRed Nucleic Acid Gel Stain (Biotium, Hayward, CA, USA). The gel was allowed to set for at least 25 min at room temperature. The gel was then placed in a gel electrophoresis tank and 1×TAE running buffer (pH 7.4) was used. Before loading, the samples were incubated at 40° C. with or without 2% of Triton X-100, for total and free RNA, respectively. The gel was run at 80 V for 40 minutes. Gel images were taken on a Chemidoc XRS imaging system (Bio-Rad, Berkeley, CA, USA).

### Hemolysis Assay

A hemolysis assay was performed to study the hemolytic properties of the formulations. For this goal Human blood diluted in 1×PBS to a 20% vol/vol blood solution. In a 96-round bottom plate, 100 μL LNPs diluted at an equivalent concentration of 0.05 mg mRNA/mL were added to 100 μL blood solution in PBS and incubated at 37° C. for 1 h (triplicates). After incubation, the plate was centrifuged at 23° C. at 500×g for 5 min. And then, the supernatant was transferred into a clear 96-well plate and UV absorption was read at 540 nm using Tecan Pro200 Plate Reader. Positive and negative controls were carried out with 0.2% Triton-X and buffer alone, respectively.

### Complement Activation Assay

In vitro SC5b-9 levels were determined by by Microvue SC5b-9 Plus ELISA kit (Quidel Co., SanDiego, CA, USA). Briefly, samples and positive (Cremophor El, Merck, Darmstadt, Germany; Cobra venom factor; Quidel Co., SanDiego, CA, USA) and negative (1×DPBS and Intralipid) controls were incubated with Normal Human Serum Complement (Innovative Research, Michigan, USA) at the ratio of 20:80 (Specimen: Serum) for 1 h at 37° C. LNP formulations were incubated with human serum at a final mRNA concentration of 0.01 mg/ml, the theoretical concentration (corresponding to a dose of 0.49 mg/kg). SC5b-9 EIA kit (Quidel, San Diego, USA) was executed according to manufactures protocol.

### Cells and Cell Lines in Potency Assay

HEK 293T-17 cells were seeded into 12-well plates (4e5 cells/well in DMEM (#31966-021/2293715 Gibco)+10% FCS (S 0615/BS.1640839, Sigma)) 6 h prior to transfection. Formulations were diluted in Opti-MEM (#51985-026, Gibco) and 0.3 μg/well were used to transfect the cells in triplicates. Afterwards, cells were centrifuged at 300×g for 4 min. As a transfection and/or staining control, two standard LNPs containing RNA coding for cGFP (LNP-315-d045, 29th April 2021, BNT Delivery Technologies GmbH, Halle) or the S protein of SARS-COV-2 (RBP020.LNP, COVVAC/270320, Polymun, Klosterneuburg) were used. Cells were incubated at 37° C./5% CO₂ for 18 h. For FACS analysis, cells were detached using PBS ((#2339094, Gibco), 4° C.) and transferred into a 96-well plate. Cells were stained for viability with a 1:500 dilution of the fixable viability dye eFluor 450 (#65-0863-14, eBioscience), followed by fixation (#42081, Biolegend) and permeabilization (1× dilution, #00-8333-56, eBioscience). For the quantification of S1 protein expression, cells were stained with a 1:2000 dilution of the primary antibody (#40150-R007, Sino Biological) followed by secondary antibody staining (1:1000, #P-2771MP, ThermoFischer). Cells were resuspended in FACS buffer (PBS, EDTA, BSA) for acquisition. To that end, cell viability, transfection frequencies and MFI of the over-all and the S1-poitive population were determined and compared.

### Cells and Cell Lines in Luciferase Assay

Luciferase assays are a reliable and sensitive method to detect the expression of luciferase in live cells.

In this assay, firefly luciferase catalyse the mono-oxygenation of beetle luciferin via ATP and Mg2+-dependent. This results in emission of light in the range of 550 to 620 nm. For cell transfections, HepG2, C2C12 and RAW 264.7 cells were seeded at respective cell number of 20000 cells/well, 5 000 cells/well and 40 000 cells/well in Nunc white flat-bottom 96-well plate (Merck KGaA, Darmstadt, Germany) and centrifuged at 500×g for 5 min. After 18-24 h, the medium was replaced by fresh medium at 90 μl/well DPBS and only medium were used as negative and blank control, respectively. Formulation samples were tested at mRNA assay concentration between 50 to 125 ng per well. Samples and controls were added at 10 μl/well. After 24 h, luciferase expression was determined by Bright-Glo™ Luciferase assay (Promega, Madison, WI. USA) according to manufacturer's protocol. Briefly, the reagent was added to the cells in medium in 1:1 (v/v) ratio, followed by an incubation of 5 min in the dark to allow for complete cell lysis. Viability was measured by CellTiter-Glo® (Promega, Madison, WI, USA) according to manufacturer's protocol. Briefly, the reagent was added to the cells in medium in 1:1 (v/v) ratio, followed by an incubation of 10 min in shaker followed by 20 min to allow for stabilization the signal. Alternatively, luciferase and viability was determined by ONE-Glo™+Tox Luciferase Reporter and Cell Viability Assay (Promega GmbH, Madison, WI. USA) according to manual instructions. Briefly, 20 μl of 5× CellTiter-Fluor™ Reagent was added to the wells, mix by orbital shaking (300-500 rpm for ˜30 sec). After incubation for 30 min at 37° C., the fluorescence was measured with excitation wavelength at 400 nm and emission at 505 nm (viability). Then, ONE-Glo™ Reagent was added at 100 μl/well. After incubation for three min, the bioluminescense was measured (luciferase expression). Bioluminescence signals (photons per second [p/s])) and fluorescence were measured using a microplate luminescence reader Infinite M200 (Tecan, Männedorf, Switzerland). Relative luminescence was calculated by subtracting the signal of DPBS control from the sample control. Relative viability was calculated by:

$$Viability ( \% ) = \frac{( RLU sample - RLU blanck )}{( RLU PBS - RLU blanck )} \times 100$$

### Animal Experiments

Healthy female Balb/C mice were obtained from Charles River. Group size was 6 per test item for intravenous (i.v.) experiment. The total number of test groups was 7. For biodistribution studies, lipid nanoparticles were injected i.v. in a volume of 100 μL (2 μg mRNA). Biological activity of luciferase-encoding mRNA was evaluated by ventral bioluminescence imaging using an IVIS spectrum imaging system (Caliper Life Sciences, Alameda, CA, USA) Bin 4 (1 min) and Bin 8 (1 min). Readout was performed after 0 h, 6 h and 24 h. Bioluminescence was presented as color-scaled images superimposed on grayscale photos. Quantification resulted from the average radiance (quantified as photons/see/cm²/sr) from each pixel inside the regions of interest (ROIs).

### Materials

Cholesterol and distearoyl-sn-glycerol-3-phosphocholine (DSPC) were supplied from Avanti (Alabama, USA). The cationically ionizable lipids HY-501 (also designated as XIV-3 herein) and HY-405 (also designated as XIV-1 herein) were purchased from NucleoSyn (Olivet, France). Ethanol absolute was purchased by Merck Millipore. OmniPur® Water (WFI Quality, sterile purified water) was purchased from Merck Millipore. The RNase-free TE buffer concentrate, 20× used for the RiboGreen® RNA quantitation assays, as well as the Ribogreen dye were purchased from Invitrogen. Triton X-100 was purchased from VWR International GmbH, Darmstadt, Germany. All materials in contact with the solutions and LNP preparations were sterile. All methods including the use of kits and reagents are carried out according to the manufacturer's information unless specifically indicated. Luciferase-encoding mRNA was used. The mRNA was synthesized in house using internal protocols.

The polyoxazoline (POX)-grafted lipids were supplied by Avroxa BV (Ghent, Belgium). The list and structure of POX-conjugated lipids used are summarized in the following table and were mostly provided by Avroxa BV. The indices 25, 45, and 50 in these POX-conjugated lipids mean that the POX-conjugated lipids contain about 20-25 (index 25) and 45-50 (index 45 or 50) repeating units, respectively.

| Name | Chemical Structure |
|---|---|
| C14- PMeOX25- NH2 | 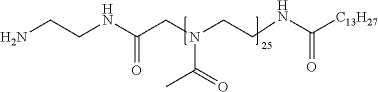 |
| C14- PMeOX25- COCH3 | 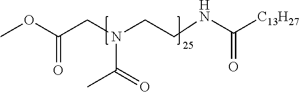 |
| C14- PMeOX25- COOH | 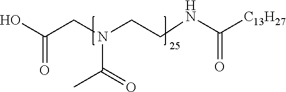 |
| C14- PMeOx25- OH | 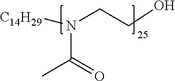 |
| C14- PMeOX45- NH2 | 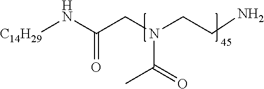 |
| C14- PMeOX45- COOH | 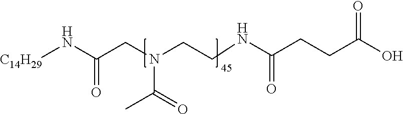 |
| C14- PMeOX45- OH | 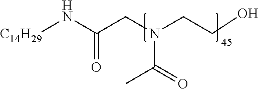 |
| C14- PMeOX45- NH—AC | 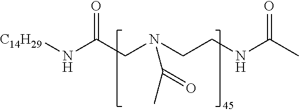 |
| VE- PMeOX50- CH3 | 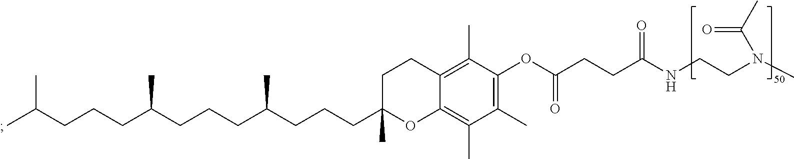 |

The POX-grafted lipids may be prepared using the scheme shown in FIG. 1. For example, the synthesis of the POX-grafted lipid C14-PMcOx25-OH may be conducted according to the following reaction:

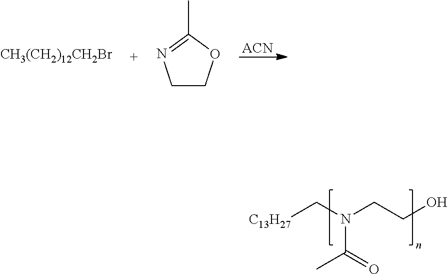

Briefly, freshly distilled monomer (e.g., 2-methyl-4,5-dihydro-1,3-oxazole or 2-ethyl-4,5-dihydro-1,3-oxazole), solvent (e.g., dry acetonitrile (ACN)), and initiator (e.g., freshly distilled 1-bromotetradecane) are added to a screw-cap tube under nitrogen purge, the tube is sealed, and left stirring under microwave irradiation at 140° C. for 15 min. The amount of initiator (mole ratio of initiator to monomer) is determined by the desired degree of polymerization. Termination can be performed by adding a methanolic solution of KOH (0.5 M) and stirring at 25° C. for 2 h which causes the conversion of the terminal oxazolinium ion into an N-(2-hydroxyethyl) group. Purification by dialysis (100-500 MWCO membrane) results in the desired POX-grafted lipid.

For the synthesis of a POX-grafted lipid of formula (II), the following scheme can be used:

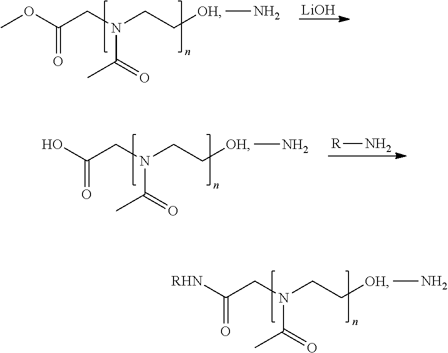

wherein R is a hydrophobic chain such as C₁₄H₂₉. Briefly, freshly distilled monomer (e.g., 2-methyl-4,5-dihydro-1,3-oxazole), solvent (e.g., dry acetonitrile), and initiator (e.g., distilled methyl 2-bromoacetate) are added to a screw-cap tube under nitrogen purge, the tube is sealed, and left stirring under microwave irradiation at 140° C. for 15 min. The amount of initiator (mole ratio of initiator to monomer) is determined by the desired degree of polymerization. Termination can be performed by adding a methanolic solution of KOH (0.5 M) and stirring at 25° C. for 2 h which causes the conversion of the terminal oxazolinium ion into an N-(2-hydroxyethyl) group. Alternatively, termination can be performed by adding a methanolic solution of NaN₃ and stirring at 25° C. for 2 h which causes the incorporation of the terminal azide group (which can be reduced to an amine group in a later step). The polymer is purified by precipitation in diethyl ether. Treatment with an amine containing one or more hydrophobic chains (such as myristyl amine), in the presence of coupling additives (such as N-hydroxysuccinimide and/or dicyclohexylcarbodiimide), results in the —OH or —N₃ terminated POX-grafted lipid.

Subsequently, the azide group is reduced into amine using a suitable reducing agent (e.g. triphenylphosphine). Purification by dialysis (100-500 MWCO membrane) results in the desired amine. The hydroxy or amine terminated polymer may be dissolved in an aqueous solution of lithium hydroxide (1.0 M), and reacted overnight at room temperature. The product (i.e., the free carboxylic acid) may be dialyzed with a 100-500 MWCO dialysis membrane.

A carboxyl-terminated POX-grafted lipid can be obtained by reaction of an amine-terminated POX-grafted lipid (such as the amine-terminated POX-grafted lipid disclosed above) with succinic anhydride.

For the synthesis of a POX-grafted lipid of formula (II′), the following scheme can be used:

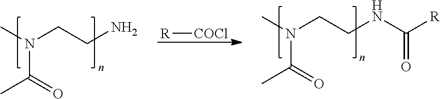

Briefly, freshly distilled monomer (e.g., 2-methyl-4,5-dihydro-1,3-oxazole), solvent (e.g., dry acetonitrile), and initiator (e.g., distilled methyl p-toluenesulfonate) are added to a screw-cap tube under nitrogen purge, the tube is sealed, and left stirring under microwave irradiation at 140° C. for 15 min. The amount of initiator (mole ratio of initiator to monomer) is determined by the desired degree of polymerization. Termination can be performed by adding a methanolic solution of NaN₃ and stirring at 25° C. for 2 h which causes the incorporation of the terminal azide group. The polymer is purified by precipitation in diethyl ether. Subsequently, the azide group is reduced into amine using a suitable reducing agent (e.g. triphenylphosphine). Purification by dialysis (100-500 MWCO membrane) results in the desired amine terminated poly(2-alkyl-2-oxazoline). Then, the amine is reacted with triethylamine followed by an acid halide (e.g., myristoyl chloride), dropwise at 0° C. and under an argon flow. The solution is let to react for 48 h at room temperature. The product is purified by precipitation in diethyl ether and further by flash chromatography, yielding the desired POX-grafted lipid of formula (II′).

Further POX-grafted lipids may be prepared by derivatizing the terminal group. E.g., by using methyl 2-bromoacetate as initiator, 2-methyl-4,5-dihydro-1,3-oxazole as moinomer, and NaN₃ as termination agent the following polymer can be synthesized (after the azide group has been reduced to an amine group):

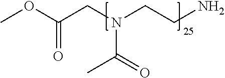

After reaction with an acyl halide (R—COCl, wherein R is a hydrophobic chain such as C₁₋₃H₂₇), the resulting POX-grafted lipid of formula (II′) (cf. compound 1 in the following scheme) can be derivatized into further POX-grafted lipids of formula (II′) (cf., compounds 2 and 3 in the following scheme).

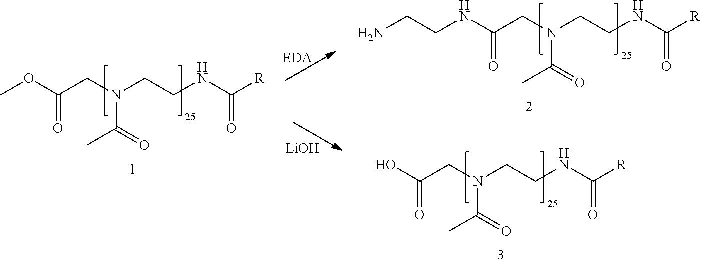

For example, compound 1 can be reacted with ethylenediamine (EDA) for 3 days at 70° C. and purified by precipitation in diethyl ether to yield compound 2. Compound 3 may be obtained by reaction of compound 1 with an aqueous solution of lithium hydroxide (1.0 M) overnight at room temperature. The product can be dialyzed with a 100-500 MWCO dialysis membrane.

### Example 1

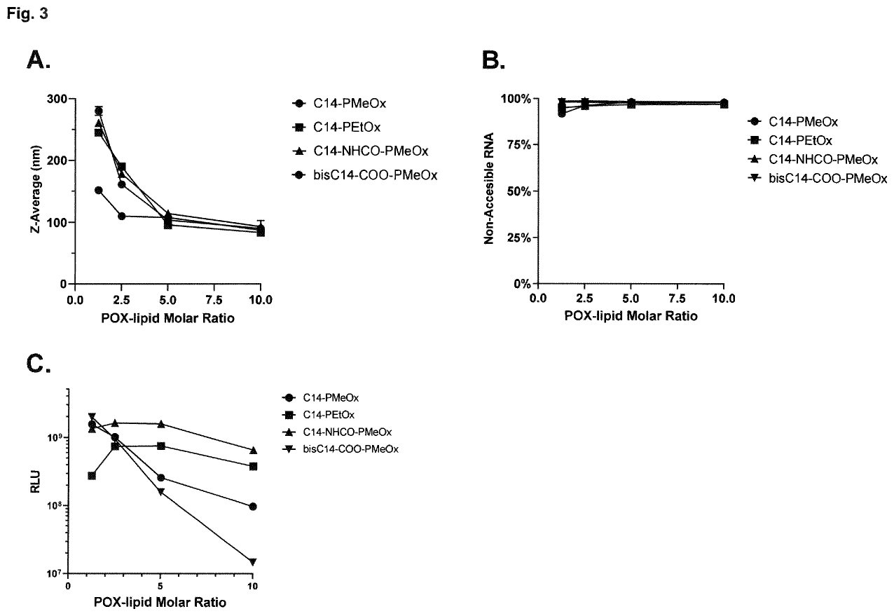

This experiment has been conducted to evaluate the grafting % of poly-(2-oxazoline)-grafted-lipids into LNPs. To this end, four different poly-(2-oxazoline)-grafted-lipids (hereinafter POX-lipids) were used to form lipid nanoparticles, whereas the commonly used polyethylenglycol-grafted-lipid is substituted by a POX-lipid. LNPs were prepared by mixing with a microfluidic system (Nanoassembler). In this process, an aqueous phase (0.2 mg/mL of modified mRNA (coding for luciferase) and 100 mM citrate buffer at pH 4.0) and an organic phase (a lipid mixture consisting of DODMA, cholesterol, DOPE and a POX-Lipid dissolved in ethanol) were mixed. The molar ratio of the lipid mixture for each tested LNP was calculated by the ratio 40:48-Y: 10:Y (DODMA:Cholesterol:DOPE:POX-lipid, wherein Y represents the molar ratio of POX-lipid). Four different POX-Lipids were tested: C14-PMeOx (a tetradecyl alkyl chain followed by 45-50 units of poly-2-methyl-2-oxazoline), C14-PEtOx (a tetradecyl alkyl chain followed by 45-50 units of poly-2-ethyl-2-oxazoline), C14-NHCO—PMeOx (a tetradecyl alkyl chain linked by an amide bond to a block of 45-50 units of poly-2-methyl-2-oxazoline) and bisC14-COO—PMeOX (two tetradecyl alkyl chains, each linked via ester bonds to one single block of 45-50 units of poly-2-methyl-2-oxazoline). All LNPs were formulated at a N/P ratio of 4, whereas the N/P ratio represents the number of amine groups (present in ionizable lipid) to phosphate groups (present in the RNA backbone) in formulated LNPs. The mixing of the aqueous phase and organic phase was performed at a volume ratio of 3:1 (aqueous:organic) and at a mixing speed of 12 mL/min. After mixing, the LNP containing solution was dialyzed in a slide-a-lyzer 10k MWCO dialysis cassette against a 10% w/v sucrose solution with 20 mM HEPEs at pH 7.1 for 3 h at room temperature and then for 12 h overnight at 4° C. After the dialysis the LNPs were evaluated regarding their size and RNA accessibility using standard protocols. FIG. 3A indicates the size of the LNPs with four different POX-lipids at 4 different ratios of POX-lipid each. FIG. 3B indicates the quantified amount of non-accessible RNA in the LNPs with four different POX-lipids at 4 different ratios of POX-lipid each. FIG. 3C indicates luciferase expression as area under the curve after 24 h of transfection of C2C12 for the different tested LNPs within a dose range between 12.5 ng and 400 ng of RNA/well.

### Example 2

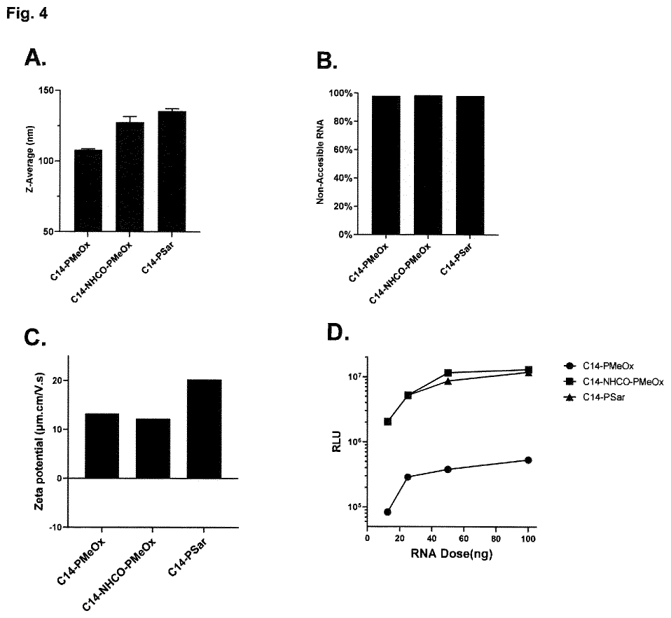

The aim of this experiment was to determine the impact of the introduction of a polar linker at the intersection between the hydrophilic and hydrophobic blocks in POX-lipids. To this end, two different POX-lipid were used to form lipid nanoparticles, whereas an newly described polysarcosine-grafted-lipid (hereinafter PSar-Lipid) is used as a reference (Nogueira et al. 2020, DOI: 10.1021/acsanm.0c01834). LNPs were prepared by using a microfluidic system (Nanoassembler). In this process, an aqueous phase (containing 0.2 mg/mL of modified mRNA (coding for luciferase) and 100 mM citrate buffer at pH 4.0) was mixed with an organic phase (containing a lipid mixture of DODMA, cholesterol, DOPE and stealth-grafted-lipid dissolved in ethanol). The molar ratio of the lipid mixture used was 40:48:10:5 (DODMA:cholesterol:DOPE:stealth-grafted-lipid). The stealth-grafted-lipids used were (1) the POX-lipid C14-PMeOx (a tetradecyl alkyl chain followed by 45-50 units of poly-2-methyl-2-oxazoline); (2) the POX-lipid C14-NHCO—PMeOx (a tetradecyl alkyl chain linked by an amide bond to a block of 45-50 units of poly-2-methyl-2-oxazoline); and (3) the PSar-lipid C14-PSar (a tetradecyl alkyl chain followed by 23 units of poly(N-methyl-glycin)). All LNPs were formulated at a N/P ratio of 4, whereas the N/P ratio represents the ratio of amine (present in ionizable lipid) to phosphate (present in the RNA backbone) in the LNP formulations. The mixing of the aqueous and organic phases was conducted at a volume ratio of 3:1 (aqueous:organic) and at a mixing speed of 12 mL/min. After mixing, the LNP formulations were dialyzed in a slide-a-lyzer 10k MWCO dialysis cassette against a solution of 10% w/v sucrose and 20 mM HEPEs at pH 7.1 for 3 h at room temperature and then for 12 h overnight at 4° C. After dialysis, using standard protocols, the LNP formulations were analysed with respect to their size, RNA accessibility, zeta potential, and in vitro luciferase expression. FIG. 4A indicates the size of the LNPs with four different POX-lipids at 4 different ratios of POX-lipid each. FIG. 4B indicates the quantified amount of non-accessible RNA in the LNPs with four different POX-lipids at 4 different ratios of POX-lipid each. FIG. 4C indicates the surface charge of the different LNP formulations. FIG. 4D indicates luciferase expression after 24 h of transfection of C2C12 cells with the different tested LNP formulations within a dose range between 12.5 ng and 100 ng of RNA/well.

As can be seen from FIGS. 4A-D, the POX-lipids achieve results with respect to size, accessible RNA, and zeta potential which are comparable to those of the reference polysarcosine-grafted-lipid. Furthermore, the POX-lipid comprising a polar linker provides higher expression compared to the POX-lipid lacking a polar linker; cf., FIG. 4D.

### Example 3

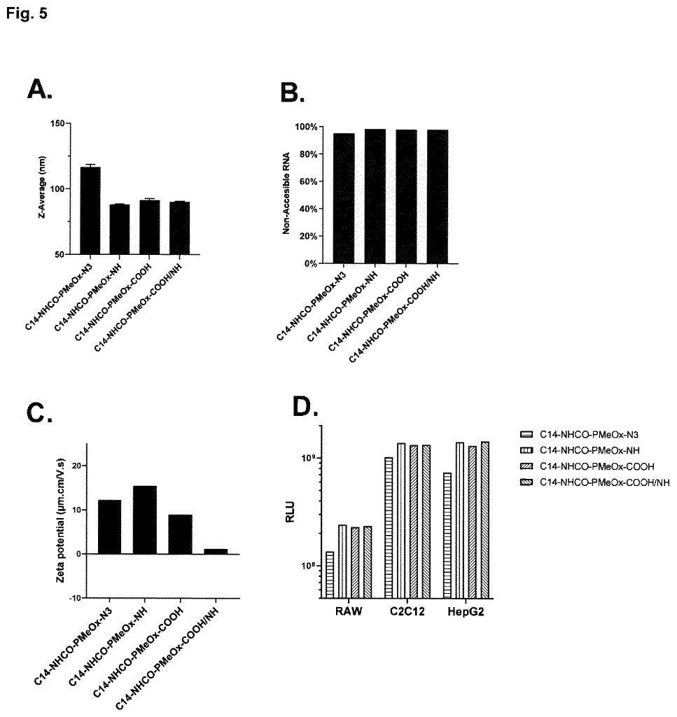

This experiment has been conducted to determine the effect of the end-group in POX-lipids. In particular, four different POX-lipid were used to form lipid nanoparticles: the POX-lipid C14-NHCO—PMeOx-N₃ (a tetradecyl alkyl chain linked by an amide bond to a block of 45-50 units of poly-2-methyl-2-oxazoline terminated by an azido group); the POX-lipid C14-NHCO—PMeOx-NH (a tetradecyl alkyl chain linked by an amide bond to a block of 45-50 units of poly-2-methyl-2-oxazoline terminated by a primary amino group); the POX-lipid C14-NHCO—PMeOx-COOH (a tetradecyl alkyl chain linked by an amide bond to a block of 45-50 units of poly-2-methyl-2-oxazoline terminated by a carboxyl group); and the POX-lipids C14-NHCO—PMeOx-COOH/NH (a physical mixture on a 1:1 ratio of C14-NHCO—PMeOx-COOH and C14-NHCO—PMeOx-NH). LNPs were prepared by using a microfluidic system (Nanoassembler). In this process, an aqueous phase (0.2 mg/mL of modified mRNA (coding for luciferase) and 100 mM citrate buffer at pH 4.0) was mixed with an organic phase (comprising a lipid mixture containing DODMA, cholesterol, DOPE and POX-lipid dissolved in ethanol). The molar ratio of the lipid mixture used was 40:48:10:5 (DODMA:cholesterol:DOPE:POX-lipid). All LNP formulations were formulated at a N/P ratio of 4, whereas the N/P ratio represents the ratio of amine (present in ionizable lipid) to phosphate (present in the RNA backbone) in formulated LNPs. The mixing of the aqueous phase and organic phase was conducted at a volume ratio of 3:1 (aqueous:organic) and a mixing speed of 12 mL/min. After mixing, the LNP formulations were dialyzed in a slide-a-lyzer 10k MWCO dialysis cassette against a solution of 10% w/v sucrose with 20 mM HEPEs at pH 7.1 for 3 h at room temperature, then for 12 h overnight at 4° C. After dialysis, using standard protocols, the LNP formulations were analysed with respect to their size, RNA accessibility, zeta potential, and in vitro luciferase expression. FIG. 5A shows the size of the LNPs with four different POX-lipids at 4 different ratios of POX-lipid each. FIG. 5B depicts the quantified amount of non-accessible RNA in the LNPs with four different POX-lipids at 4 different ratios of POX-lipid each. FIG. 5C indicates the surface charge of the different LNP formulations. FIG. 5D shows the in vitro luciferase expression as area under the curve after 24 h of transfection of cells (C2C12, HepG2 or RAW 164.7 cells) with the different tested LNPs within a dose range between 12.5 ng and 400 ng of RNA/well.

As can be seen from FIGS. 5A-D, the POX-lipids tested achieve comparable results irrespective of the particular end-group.

### Example 4

The objective of this experiment was the investigation of C14-PMeOX25-NH₂ lipid as stealth moiety for engineering mRNA nanoparticles using the LNP technology comprising HY501 as an ionizable lipid. The C14-PMeOX25-NH₂ containing LNPs were prepared by mixing the RNA aqueous phase and the lipid organic phase using a microfluidic instrument. Different mole fraction of C14-PMeOX25-NH2 lipid (2-6%) were used for LNP formulations. The ionizable lipid HY501, the helper lipid DSPC, and cholesterol at respective molar fractions 47.5:42.5-X: 10 was used, where x is the fraction of the C14-PMeOX25-NH₂ lipid. N/P ratio of 6 was selected for the experiments. Moreover, the physicochemical properties and biological performance of these C14-PMeOX25-NH₂ containing formulations were compared with other PEG containing BM_2 formulation. A summary of all the formulations used in this experiment is given in Table 1 (for all formulations the N/P ratio was 6, the RNA was mod. RNA luciferase, the RNA buffer was 100 mM CB pH 4.0, the dialysis buffer was PBS, and the final concentration was 0.05 mg/mL).

TABLE 1
Design of LNP formulations containing C14-PMeOX25-NH2.

| LNP | | |
|---|---|---|
| number | LNP name | Formulation composition/mol % |
| LNP#01 | BM_2 | — |
| LNP#02 | HY501-C14-PMeOX25-NH2_6 | HY501:CHOL:DSPC:C14-PMeOX25-NH2 (47.5:36.5:10:6) |
| LNP#03 | HY501-C14-PMeOX25-NH2_5 | HY501:CHOL:DSPC:C14-PMeOX25-NH2 (47.5:37.5:10:5) |
| LNP#04 | HY501-C14-PMeOX25-NH2_4 | HY501:CHOL:DSPC:C14-PMeOX25-NH2 (47.5:38.5:10:4) |
| LNP#05 | HY501-C14-PMeOX25-NH2_3 | HY501:CHOL:DSPC:C14-PMeOX25-NH2 (47.5:39.5:10:3) |
| LNP#06 | HY501-C14-PMeOX25-NH2_2 | HY501:CHOL:DSPC:C14-PMeOX25-NH2 (47.5:40.5:10:2) |

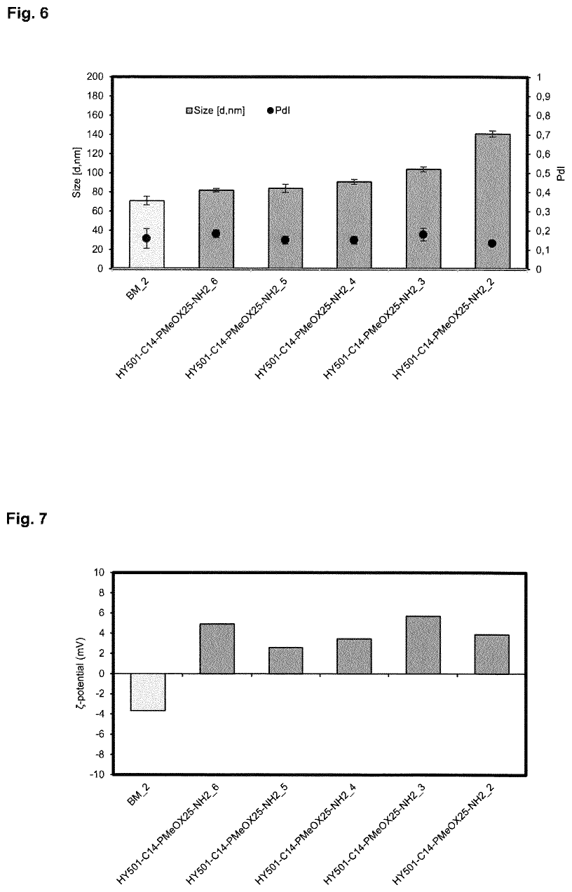

The evolution of size and size distribution of C14-PMeOX25-NH₂ containing LNPs (based on HY-501) manufactured with different concentration of C14-PMeOX25-NH₂ lipid in the formulation are shown in FIG. 6. As can be seen, the particle sizes decreased monotonously from 141 to 82 nm with the increasing amounts of C14-PMeOX25-NH₂ lipid from 2 to 6 mol % in LNP compositions. On the other hand, all the formulations exhibited a PdI inferior to 0.2.

FIG. 7 shows the zeta potential of BM_2 and the C14-PMeOX25-NH₂ containing LNPs measured in 0.1×PBS at pH 7. The results show that neutral to slightly positive surface charge was measured for C14-PMeOX25-NH₂ containing LNPs. On theother hand slight negative zeta potential was measured for the BM_2 LNP formulation.

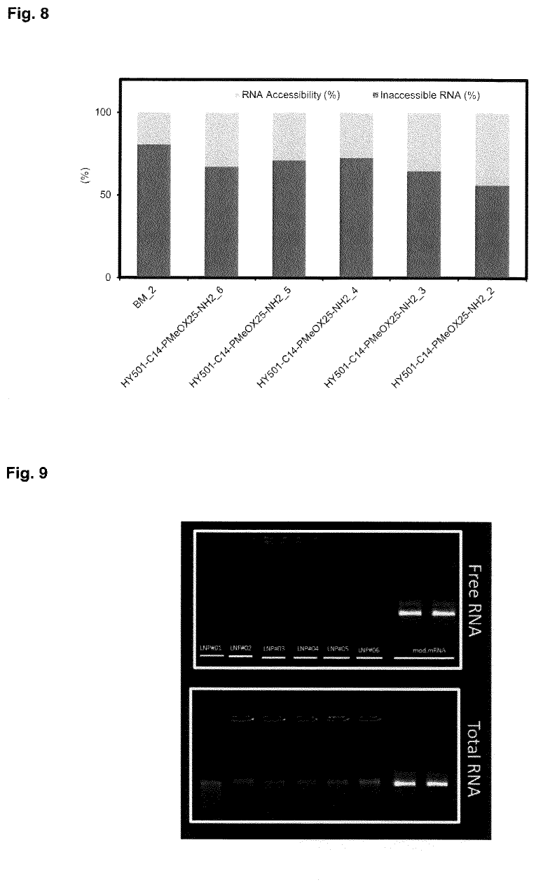

The accessibility of RNA and the amount of free RNA in the LNP formulations was measured by Ribogreen Assay and Agarose gel electrophoresis methods, respectively. FIG. 8 presents the RNA accessibility of the formulations. As can be seen, the accessibility of RNA in the formulations increased slightly with the concentration of C14-PMeOX25-NH₂ lipid in the formulation from 2 to 4 mol %. On the other hand, the Agarose gel electrophoresis images show almost no to negligible free RNA in the formulations (all <1%; FIG. 9). These results from the agarose gel measurements show that virtually almost all of RNA is bound to the particles for all the formulations.

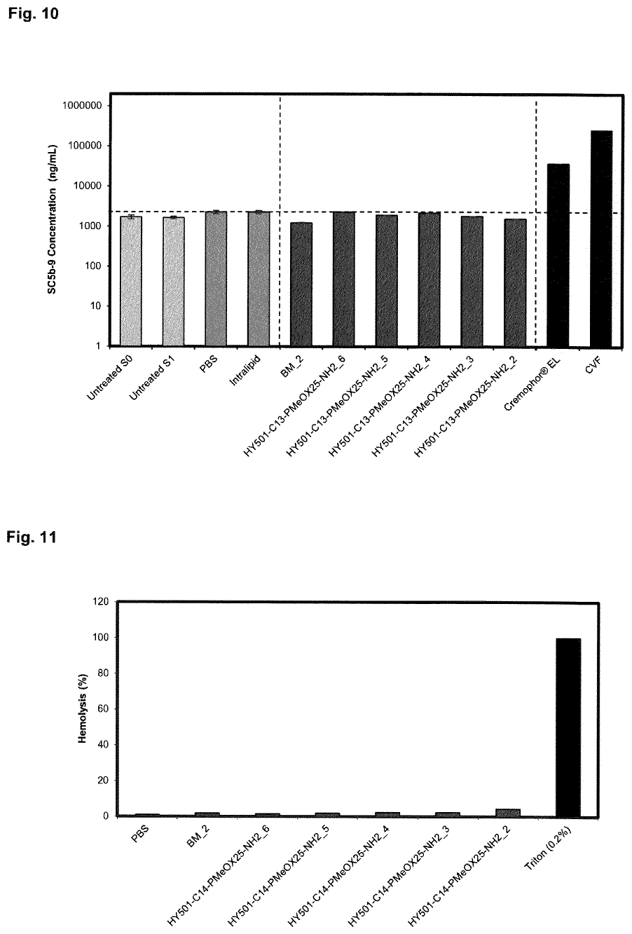

Complement activation of the formulations were studied by in vitro quantification of SC5b-9 level using Microvue SC5b-9 Plus ELISA kit (Quidel Co., SanDiego, CA, USA). FIG. 10 shows the terminal complement complex (SC5b-9) formation after incubation of human serum with LNP formulations. The concentration of formed SC5b-9 complex corresponding to the control items are also included. As can be seen no complement activation was observed for all test items.

FIG. 11 presents the hemolysis analysis after incubation (in neutral pH condition) of the whole human blood with LNP formulations. The hemolysis analysis of control items is also included. As can be seen negligible hemolysis was observed for all the tested formulation. In addition, the hemolysis of C14-PMeOX25-NH₂ containing formulations decreased with increasing the C14-PMeOX25-NH₂ lipid concentration. This is most likely due to the reduction of LNP interaction with red blood cells by increasing stealth moiety concentration in the formulations.

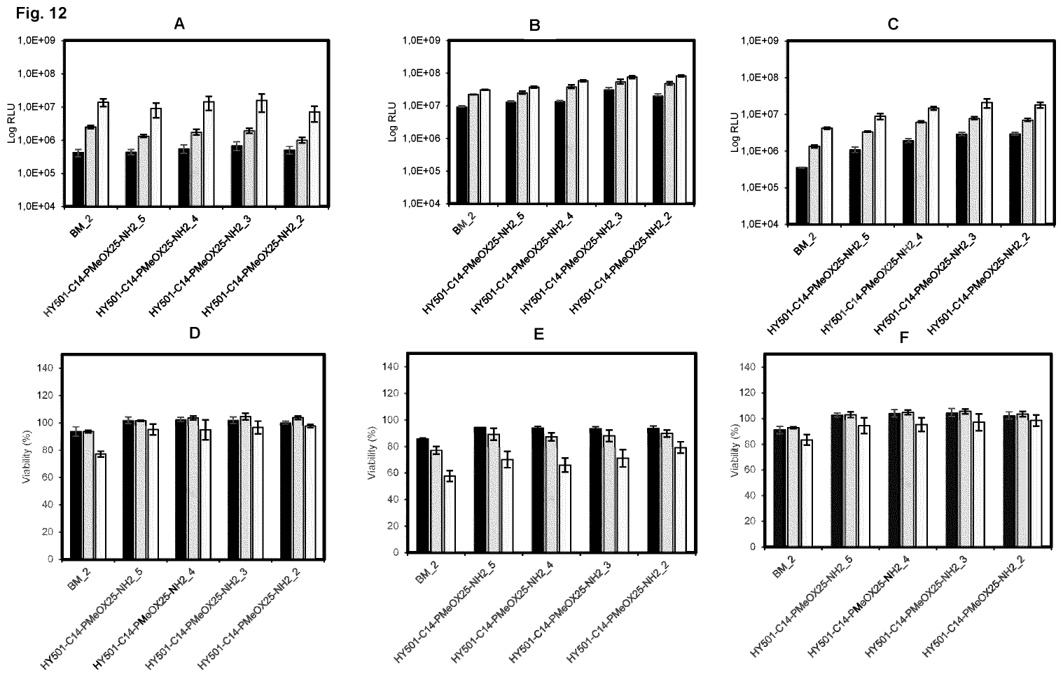

To investigate the C14-PMeOX25-NH2 LNP functionality, mRNA expression was evaluated in vitro using a skeletal muscle cell line (C2C12), a hepatocarcinoma cell line (HepG2) and a murine macrophage cell line (Raw cell). For this purpose, the LNP formulations comprising firefly luciferase-encoding mRNA were incubated with the cells at three dosages, 12.5, 25 and 50 ng per well and luciferase expression was assessed 24 h post-incubation. FIG. 12 shows that the luciferase expression (A-C) of formulation containing C14-PMeOX25-NH₂ was similar or slightly higher than the BM_2 formulations in C2C12 cell line in all tested RNA doses (C2C12: A; HepG2: B; Raw cell: C). In addition, the luciferase expression of the formulations in HepG2 and RAW cell lines was higher than BM_2 LNP. Moreover, the highest performance was observed for the formulations containing 2-3 mol % of C14-PMeOX25-NH2 in HepG2 and RAW cell lines and the performance of the formulations decreased with increasing stealth lipid concentration. On the other hand, no-to-low cytotoxicity (C2C12: D; HepG2: E; Raw cell: F) was observed for the LNP formulations in the tested doses in all tested cell lines except for 50 ng dose in HepG2 cell line.

### Example 5

The objective of this experiment was the investigation of C14-PMeOX25-COCH₃ lipid as stealth moiety for engineering mRNA nanoparticles using the LNP technology comprising HY501 as an ionizable lipid. The C14-PMeOX25-COCH₃ containing LNPs were prepared by mixing the mRNA aqueous phase and the lipid organic phase using a microfluidic system. Wide range of mole fraction of C14-PMeOX25-COCH₃ lipid (2-6%) was applied for LNP formulations. The ionizable lipid HY501, the helper lipid DSPC, and cholesterol at respective molar fractions 47.5:42.5-X: 10 was used, where x is the mole fraction of the C14-PMeOX25-COCH₃ lipid. N/P ratio of 6 was applied for all formulations. Moreover, the physicochemical properties and biological performance of these C14-PMeOX25-COCH₃ containing formulations were compared with other PEG-Lipid containing formulation (BM_2). A summary of all the formulations used in this study is given in Table 2 (for all formulations the N/P ratio was 6, the RNA was mod. RNA luciferase, the RNA buffer was 100 mM CB pH 4.0, the dialysis buffer was PBS, and the final concentration was 0.05 mg/mL).

TABLE 2
Design of LNP formulations containing C14-PMeOX25-COCH3.

| LNP | | |
|---|---|---|
| number | LNP name | Formulation composition/mol % |
| LNP#01 | BM_2 | — |
| LNP#02 | HY501-C14-PMeOX25-COCH3_6 | HY501:CHOL:DSPC:C14-PMeOX25-COCH3 (47.5:36.5:10:6) |
| LNP#03 | HY501-C14-PMeOX25-COCH3_5 | HY501:CHOL:DSPC:C14-PMeOX25-COCH3 (47.5:37.5:10:5) |
| LNP#04 | HY501-C14-PMeOX25- COCH3_4 | HY501:CHOL:DSPC:C14-PMeOX25-COCH3 (47.5:38.5:10:4) |
| LNP#05 | HY501-C14-PMeOX25- COCH3_3 | HY501:CHOL:DSPC:C14-PMeOX25-COCH3 (47.5:39.5:10:3) |
| LNP#06 | HY501-C14-PMeOX25- COCH3_2 | HY501:CHOL:DSPC:C14-PMeOX25-COCH3 (47.5:40.5:10:2) |

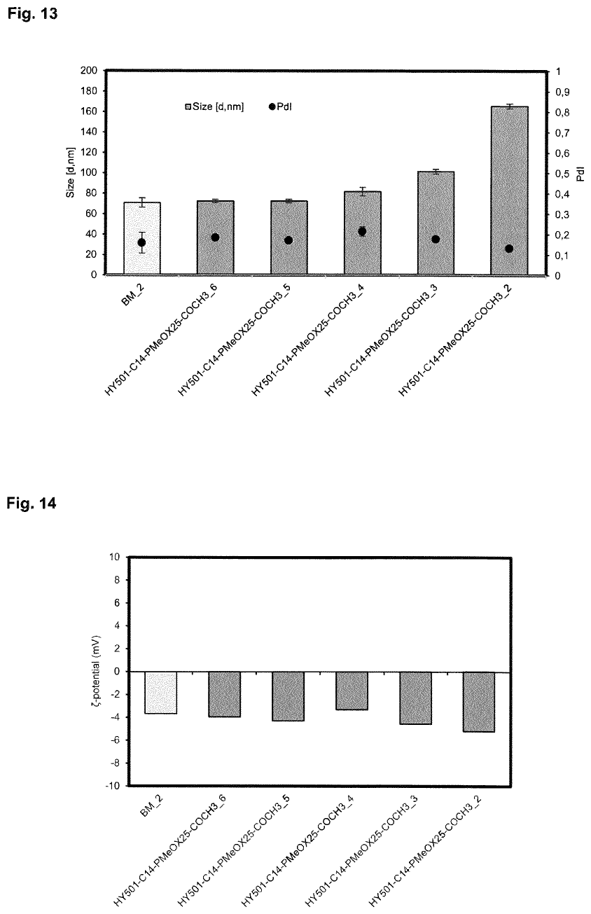

The evolution of size and size distribution of C14-PMeOX25-COCH₃ containing LNPs (based on HY-501) manufactured with different mole fractions of C14-PMeOX25-COCH₃ lipid in the formulations are shown in FIG. 13. As can be seen, the particle sizes decreased monotonously from 166 to 73 nm with the increasing amounts of C14-PMeOX25-COCH₃ lipid from 2 to 6 mol % in LNP compositions. On the other hand, all the formulations exhibited a PdI inferior to 0.2.

FIG. 14 shows the zeta potential of BM_2 and the C14-PMeOX25-COCH₃ containing LNPs measured in 0.1×PBS at pH 7. The results show slightly negative surface charge for all tested LNPs.

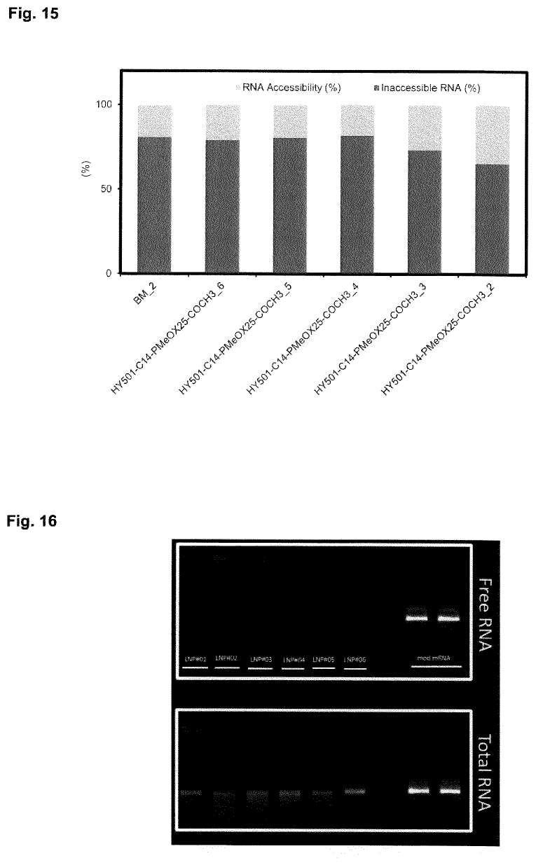

The accessibility of RNA and the amount of free mRNA in the LNP formulations were measured by Ribogreen Assay and Agarose gel electrophoresis methods, respectively. FIG. 15 presents the mRNA accessibility of the formulations. As can be seen, the accessibility of mRNA in the formulations increased slightly with the concentration of C14-PMeOX25-COCH₃ lipid in the formulation from 2 to 4 mol % and then a plateau was observed for the higher C14-PMeOX25-COCH₃ concentrations. On the other hand, the agarose gel electrophoresis images show almost no to negligible free mRNA in the formulations (all <1%; FIG. 16). These results from the agarose gel measurements show that virtually almost all of mRNA is bound to the particles for all the formulations.

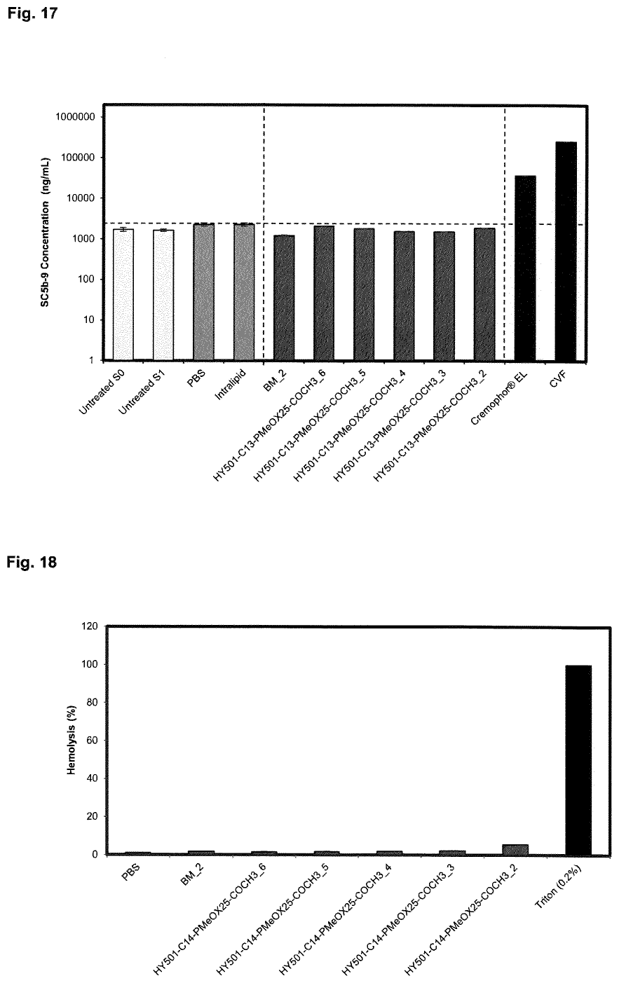

Complement activation of the formulations were studied by in vitro quantification of SC5b-9 level using Microvue SC5b-9 Plus ELISA kit (Quidel Co., SanDiego, CA, USA). FIG. 17 shows the terminal complement complex (SC5b-9) formation after incubation of human serum with LNP formulations. The concentration of formed SC5b-9 complex corresponding to the control items are also included. As can be seen no complement activation was observed for all test items.

FIG. 18 presents the hemolysis analysis after incubation (in neutral pH condition) of the whole human blood with LNP formulations. The hemolysis analysis of control items is also included. As can be seen negligible hemolysis was observed for all the tested formulation. In addition, the hemolysis of C14-PMeOX25-COCH₃ containing formulations decreased with increasing the C14-PMeOX25-COCH₃ lipid concentration. This is most likely due to the reduction of LNP interaction with red blood cells by increasing stealth moiety concentration in the formulations.

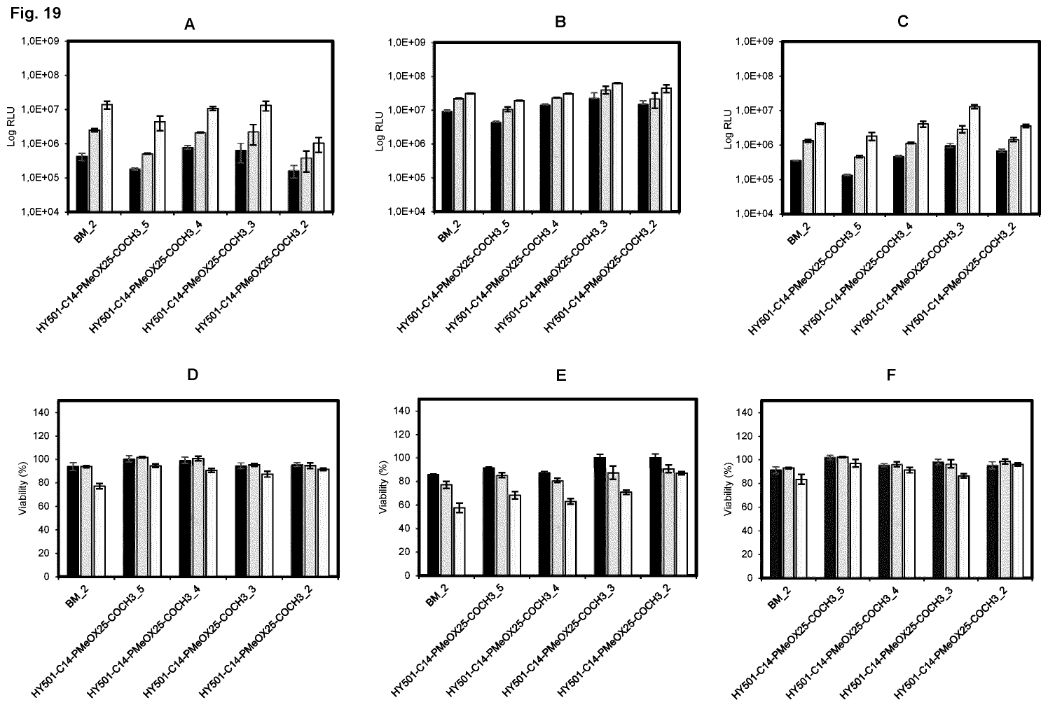

To investigate the C14-PMeOX25-COCH₃ LNP functionality, mRNA expression was evaluated in vitro using a skeletal muscle cell line (C2C12), a hepatocarcinoma cell line (HepG2) and a murine macrophage cell line (Raw cell). For this purpose, the LNP formulations comprising firefly luciferase-encoding mRNA were incubated with the cells at three dosages, 12.5, 25 and 50 ng per well and luciferase expression was assessed 24 h post-incubation. FIG. 19 shows that the luciferase expression (A-C) of formulation containing C14-PMeOX25-COCH₃ was similar or slightly higher than the BM_2 formulation in C2C12 cell line in all the tested mRNA concentrations (C2C12: A; HepG2: B; Raw cell: C). In addition, the luciferase mRNA expression of the formulations in HepG2 and RAW cell lines was higher than BM_2 LNP. Moreover, the highest performance was observed for the formulations containing 2 and 3 mol % of C14-PMeOX25-COCH₃ in HepG2 and RAW cell lines and the performance of the formulations decreased with increasing stealth lipid concentration. On the other hand, no-to-low cytotoxicity (C2C12: D; HepG2: E; Raw cell: F) was observed for the LNP formulations in the tested doses in all the cell lines except for 50 ng dose in HepG2 cell line.

### Example 6

The objective of this experiment was the investigation of C14-PMeOX25-COOH lipid as stealth moiety for engineering mRNA nanoparticles using the LNP technology comprising HY501 as an ionizable lipid. The C14-PMeOX25-COOH containing LNPs were prepared by mixing the RNA aqueous phase and the lipid organic phase using a microfluidic instrument. Different mole fraction of C14-PMeOX25-COOH lipid (2-6%) were used for LNP formulations. The ionizable lipid HY501, the helper lipid DSPC, and cholesterol at respective molar fractions 47.5:42.5-X: 10 was used, where x is the fraction of the C14-PMeOX25-COOH lipid. N/P ratio of 6 was selected for the experiments. Moreover, the physicochemical properties and biological performance of these C14-PMeOX25-COOH containing formulations were compared with other PEG containing BM_2 formulation. A summary of all the formulations used in this study is given in Table 3 (for all formulations the RNA was mod. RNA luciferase, the RNA buffer was 100 mM CB pH 4.0, the dialysis buffer was PBS, and the final concentration was 0.05 mg/mL).

TABLE 3
Design of LNP formulations containing C14-PMeOX25-COOH.

| LNP | | |
|---|---|---|
| number | LNP name | Formulation composition/mol % |
| LNP#01 | BM_2 | — |
| LNP#02 | HY501-C14-PMeOX25-COOH_6 | HY501:CHOL:DSPC:C14-PMeOX25-COOH (47.5:36.5:10:6) |
| LNP#03 | HY501-C14-PMeOX25-COOH_5 | HY501:CHOL:DSPC:C14-PMeOX25-COOH (47.5:37.5:10:5) |
| LNP#04 | HY501-C14-PMeOX25- COOH_4 | HY501:CHOL:DSPC:C14-PMeOX25-COOH (47.5:38.5:10:4) |
| LNP#05 | HY501-C14-PMeOX25- COOH_3 | HY501:CHOL:DSPC:C14-PMeOX25-COOH (47.5:39.5:10:3) |
| LNP#06 | HY501-C14-PMeOX25- COOH_2 | HY501:CHOL:DSPC:C14-PMeOX25-COOH (47.5:40.5:10:2) |

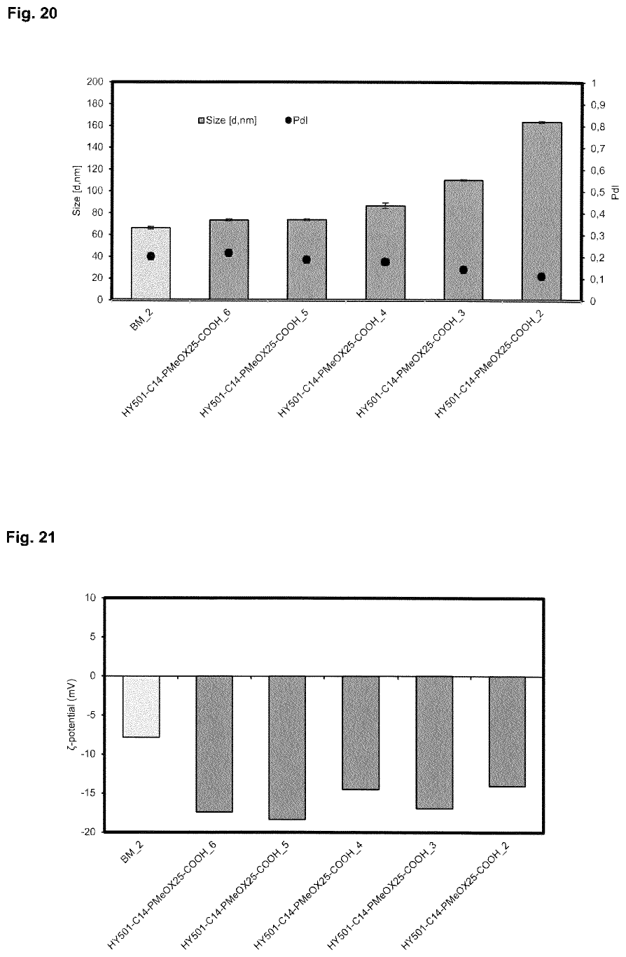

The evolution of size and size distribution of C14-PMeOX25-COOH containing LNPs (based on HY-501) manufactured with different concentration of C14-PMeOX25-COOH lipid in the formulation are shown in FIG. 20. As can be seen, the particle size decreased monotonously from 164 to 73 nm with the increasing amounts of C14-PMeOX25-COOH lipid from 2 to 6 mol % in LNP compositions. On the other hand, all the formulations exhibited a PdI inferior to 0.2.

FIG. 21 shows the zeta potential of BM_2 and the C14-PMeOX25-COOH containing LNPs measured in 0.1×PBS at pH 7. As can be seen, high negative surface charge was measured for all C14-PMeOX25-COOH containing LNPs.

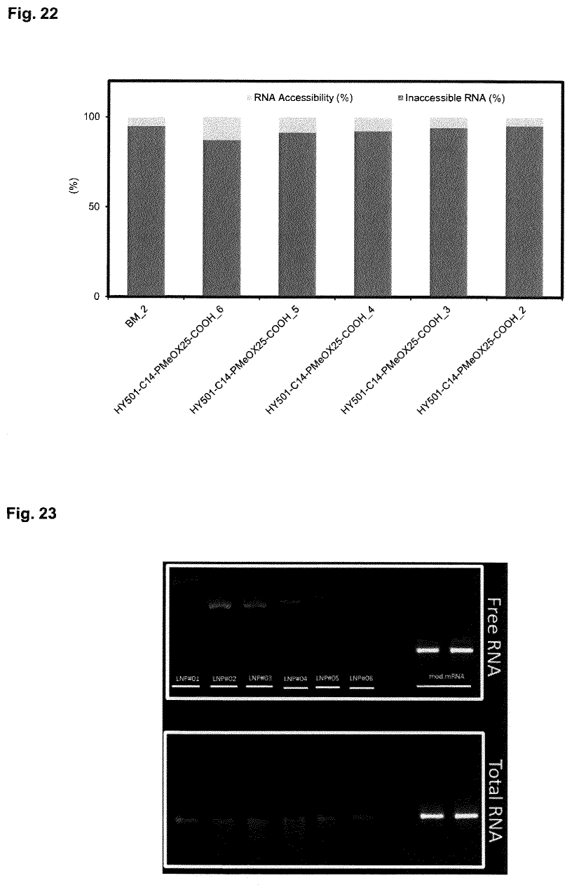

The accessibility of RNA and the amount of free RNA in the LNP formulations was measured by Ribogreen Assay and agarose gel electrophoresis methods, respectively. FIG. 22 presents the RNA accessibility of the formulations. As can be seen, high level of RNA inaccessibility was observed for all the formulations. On the other hand, the agarose gel electrophoresis images show almost no to negligible free RNA in the formulations (FIG. 23). These results from the agarose gel measurements show that virtually almost all of RNA is bound to the particles for all the formulations.

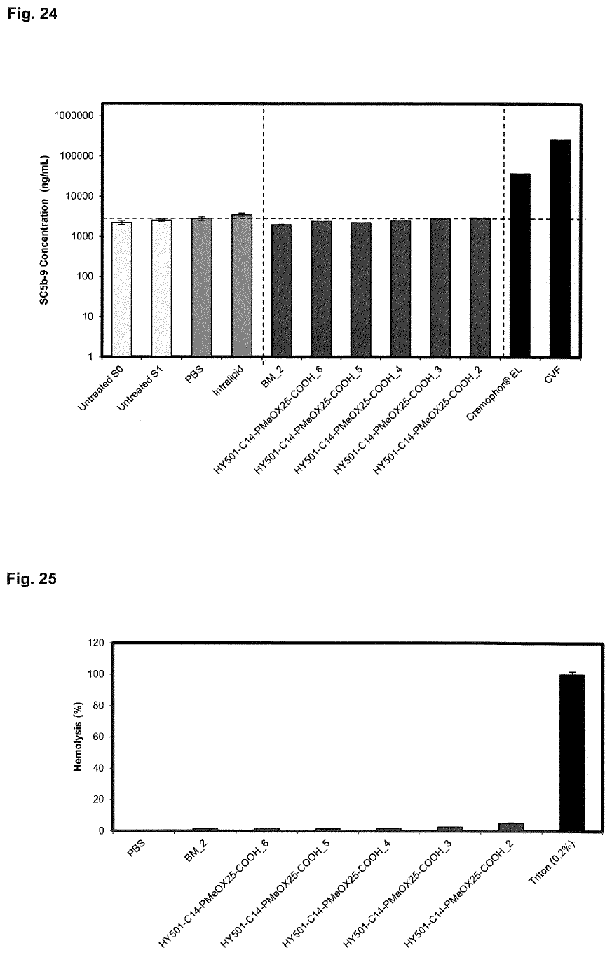

Complement activation of the formulations were studied by in vitro quantification of SC5b-9 level using Microvue SC5b-9 Plus ELISA kit (Quidel Co., SanDiego, CA, USA). FIG. 24 shows the terminal complement complex (SC5b-9) formation after incubation of human serum with LNP formulations. The concentration of formed SC5b-9 complex corresponding to the control items are also included. As can be seen no complement activation was observed for all test items.

FIG. 25 presents the hemolysis analysis after incubation (in neutral pH condition) of the whole human blood with LNP formulations. The hemolysis analysis of control items is also included. As can be seen negligible hemolysis was observed for all the tested formulation. In addition, the hemolysis of C14-PMeOX25-COOH containing formulations decreased with increasing the C14-PMeOX25-COOH lipid concentration. This is most likely due to the reduction of LNP interaction with red blood cells by increasing stealth moiety concentration in the formulations.

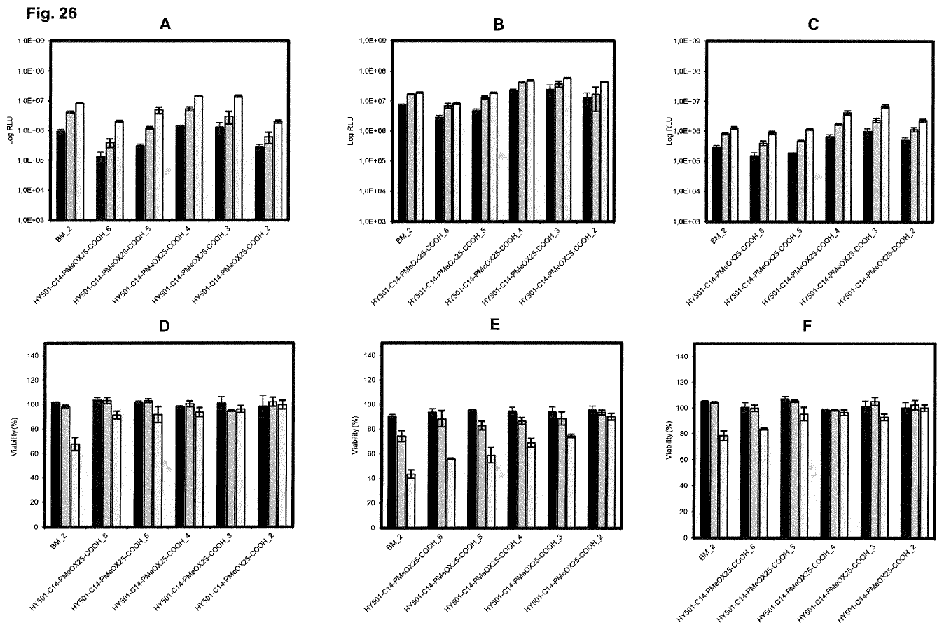

To investigate the C14-PMeOX25-COOH LNP functionality, mRNA expression was evaluated in vitro using a skeletal muscle cell line (C2C12), a hepatocarcinoma cell line (HepG2) and a murine macrophage cell line (Raw cell). For this purpose, the LNP formulations comprising firefly luciferase-encoding mRNA were incubated with the cells at three dosages, 12.5, 25 and 50 ng per well and luciferase expression was assessed 24 h post-incubation. FIG. 26 shows that the luciferase expression (A-C) of formulations containing C14-PMeOX25-COOH was similar or slightly higher than the BM_2 formulations in all the tested cell lines (C2C12: A; HepG2: B; Raw cell: C). Moreover, the highest performance was observed for the formulations containing 2-3 mol % of C14-PMeOX25-COOH and the performance of the formulations decreased with increasing stealth lipid concentration. On the other hand, no-to-low cytotoxicity (C2C12: D; HepG2: E; Raw cell: F) was observed for the LNP formulations in the tested doses in all the cell lines except for 50 ng dose in HepG2 cell line.

### Example 7

The objective of this experiment was the investigation of C14-PMeOX25-OH lipid as stealth moiety for engineering mRNA nanoparticles using the LNP technology comprising HY501 as an ionizable lipid. The C14-PMeOX25-OH containing LNPs were prepared by mixing the RNA aqueous phase and the lipid organic phase using a microfluidic instrument. Different mole fraction of C14-PMeOX25-OH lipid (2-6%) were used for LNP formulations. The ionizable lipid HY501, the helper lipid DSPC, and cholesterol at respective molar fractions 47.5:42.5-X: 10 was used, where x is the fraction of the C14-PMeOX25-OH lipid. The N/P ratio of 6 was selected for the experiments. Moreover, the physicochemical properties and biological performance of these C14-PMeOX25-OH containing formulations were compared with other PEG containing BM_2 formulation. A summary of all the formulations used in this study is given in Table 4 (for all formulations the RNA was mod. RNA luciferase, the RNA buffer was 100 mM CB pH 4.0, the dialysis buffer was PBS, and the final concentration was 0.05 mg/mL).

TABLE 4
Design of LNP formulations containing C14-PMeOX25-OH.

| LNP | | |
|---|---|---|
| number | LNP name | Formulation composition/mol % |
| LNP#01 | BM_2 | — |
| LNP#02 | HY501-C14-PMeOX25-OH_6 | HY501:CHOL:DSPC:C14-PMeOX25-OH (47.5:36.5:10:6) |
| LNP#03 | HY501-C14-PMeOX25-OH_5 | HY501:CHOL:DSPC:C14-PMeOX25-OH (47.5:37.5:10:5) |
| LNP#04 | HY501-C14-PMeOX25-OH_4 | HY501:CHOL:DSPC:C14-PMeOX25-OH (47.5:38.5:10:4) |
| LNP#05 | HY501-C14-PMeOX25-OH_3 | HY501:CHOL:DSPC:C14-PMeOX25-OH (47.5:39.5:10:3) |
| LNP#06 | HY501-C14-PMeOX25-OH_2 | HY501:CHOL:DSPC:C14-PMeOX25-OH (47.5:40.5:10:2) |

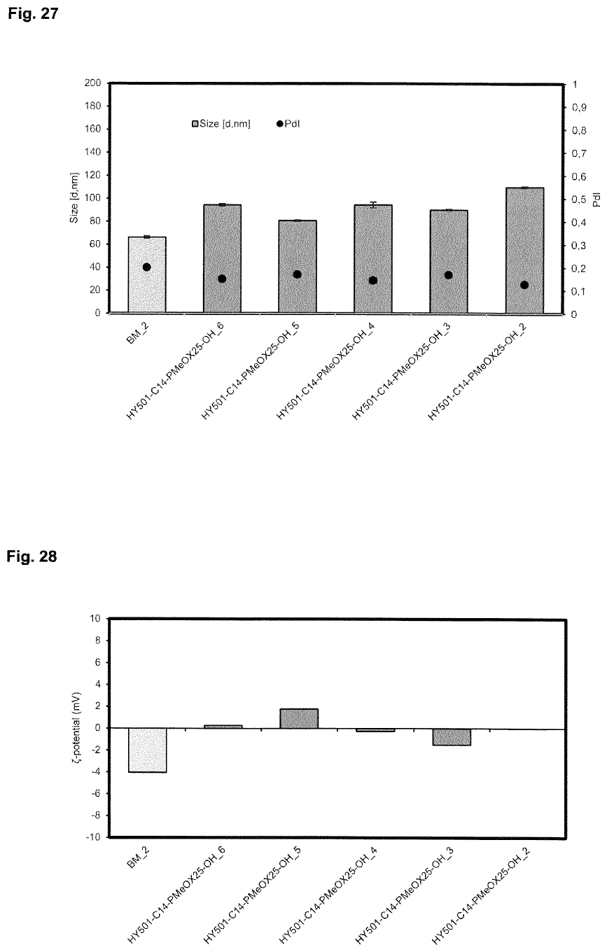

The evolution of size and size distribution of C14-PMeOX25-OH containing LNPs (based on HY-501) manufactured with different concentration of C14-PMeOX25-OH lipid in the formulation are shown in FIG. 27. As can be seen, the particle size of the formulations is in 110-80 nm range for the formulations containing 2-6 mol % of C14-PMeOX25-OH lipid. On the other hand, all the formulations exhibited a PdI inferior to 0.2.

FIG. 28 shows the zeta potential of BM_2 and the C14-PMeOX25-OH containing LNPs measured in 0.1×PBS at pH 7. As can be seen, neutral surface charge was measured for all C14-PMeOX25-OH containing LNPs.

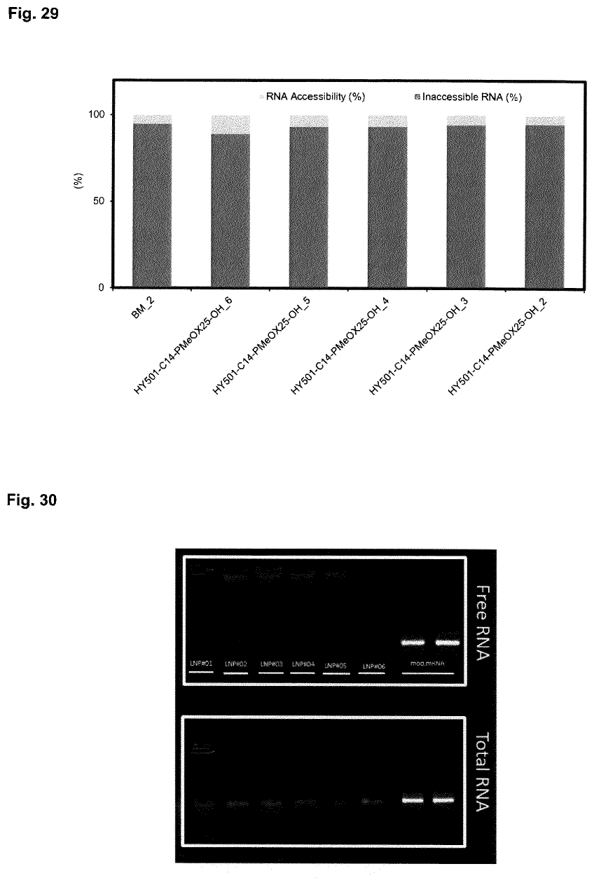

The accessibility of RNA and the amount of free RNA in the LNP formulations was measured by Ribogreen Assay and agarose gel electrophoresis methods, respectively. FIG. 29 presents the RNA accessibility of the formulations. As can be seen, high level of RNA inaccessibility was observed for all the formulations. On the other hand, the agarose gel electrophoresis images show almost no to negligible free RNA in the formulations (FIG. 30). These results from the agarose gel measurements show that virtually almost all of RNA is bound to the particles for all the formulations.

Complement activation of the formulations were studied by in vitro quantification of SC5b-9 level using Microvue SC5b-9 Plus ELISA kit (Quidel Co., SanDiego, CA, USA). FIG. 31 shows the terminal complement complex (SC5b-9) formation after incubation of human serum with LNP formulations. The concentration of formed SC5b-9 complex corresponding to the control items are also included. As can be seen no complement activation was observed for all test items.

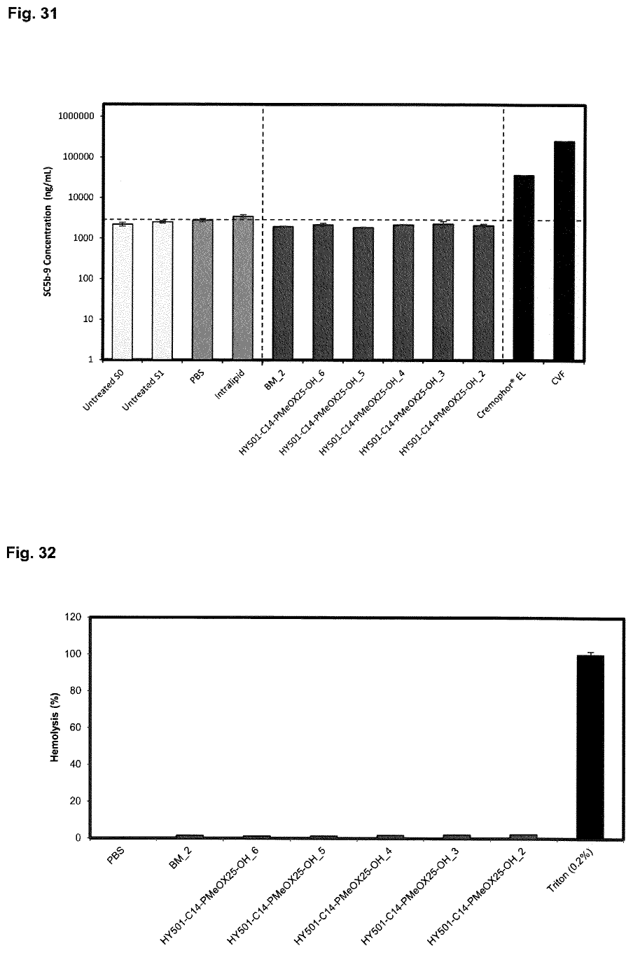

FIG. 32 presents the hemolysis analysis after incubation (in neutral pH condition) of the whole human blood with LNP formulations. The hemolysis analysis of control items is also included. As can be seen negligible hemolysis was observed for all the tested formulations. In addition, the hemolysis of C14-PMeOX25-OH containing formulations decreased with increasing the C14-PMeOX25-OH lipid concentration. This is most likely due to the reduction of LNP interaction with red blood cells by increasing stealth moiety concentration in the formulations.

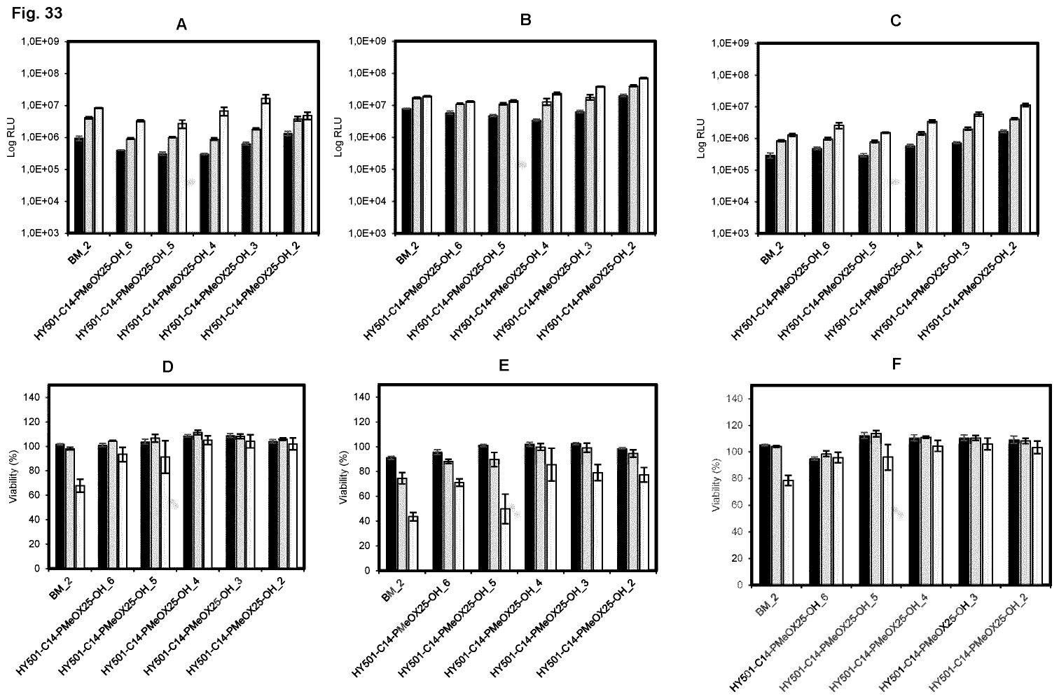

To investigate the C14-PMeOX25-OH LNP functionality, mRNA expression was evaluated in vitro using a skeletal muscle cell line (C2C12), a hepatocarcinoma cell line (HepG2) and a murine macrophage cell line (Raw cell). For this purpose, the LNP formulations comprising firefly luciferase-encoding mRNA were incubated with the cells at three dosages, 12.5, 25 and 50 ng per well and luciferase expression was assessed 24 h post-incubation. FIG. 33 shows that the luciferase expression (A-C) of formulation containing C14-PMeOX25-OH was similar or slightly higher than the BM_2 formulations in all the tested cell lines (C2C12: A; HepG2: B; Raw cell: C). Moreover, the highest performance was observed for the formulations containing 2-3 mol % of C14-PMeOX25-OH and the performance of the formulations decreased with increasing stealth lipid concentration. On the other hand, no-to-low cytotoxicity (C2C12: D; HepG2: E; Raw cell: F) was observed for the LNP formulations in the tested doses in all the cell lines except for 50 ng dose in HepG2 cell line.

### Example 8

The physicochemical properties and in vitro results of the formulations in previous parts showed a successful LNP engineering (based on HY501 as cationically ionizable lipid) with promising biological behavior for different POX-grafted lipid containing LNP formulations. The objective of this expierment was to explore the effect of POX-grafted stealth moieties on the engineering and biological behavior (in vitro and in vivo) of LNP formulations using HY501 and HY405 as cationic lipid. PMeOX25-COCH₃, PMeOX25-COOH and PMeOX25-OH stealth moieties were used in this expierment. Cationically ionizable lipid (HY-501 and HY405), the helper lipid DSPC, cholesterol and POX-grafted lipid at respective molar fractions 47.5:42.5-X: 10 was used, where x is the fraction of the POX-grafted lipid. In addition, BM_3 formulation was used as benchmark in this study. A summary of all the formulations used in this study for in vitro and in vivo (intravenous administration) is given in Table 5 (for all formulations the N/P ratiok was 6, the RNA was mod. RNA luciferase, the RNA buffer was 100 mM CB pH 4.0, the dialysis buffer was HEPES (20 mM, pH 6), the final concentration was 0.04 mg/mL, and the administration route was i.v.). Firefly-luciferase encoding mRNA was used in this experiment.

TABLE 5
Design of LNP formulations containing POX-grafted lipids for in vivo experiments.

| Group | LNP name | Formulation composition/mol % |
|---|---|---|
| 1 | BM_3 | — |
| 2 | HY501-C14-PMeOX25-COOH_4 | HY501:CHOL:DSPC:PMeOX25-COOH (47.5:38.5:10:4) |
| 3 | HY405-C14-PMeOX25-COOH_4 | HY405:CHOL:DSPC:PMeOX25-COOH (47.5:38.5:10:4) |
| 4 | HY501-C13-PMeOX25-COCH3_4 | HY501:CHOL:DSPC:PMeOX25-COCH3 (47.5:38.5:10:4) |
| 5 | HY405-C13-PMeOX25-COCH3_4 | HY405:CHOL:DSPC:PMeOX25-COCH3 (47.5:38.5:10:4) |
| 6 | HY501-C14-PMeOX25-OH_4 | HY501:CHOL:DSPC:PMeOX25-OH (47.5:38.5:10:4) |
| 7 | HY405-C14-PMeOX25-OH_4 | HY405:CHOL:DSPC:PMeOX25-OH (47.5:38.5:10:4) |

The characteristic properties of the formulation including size and size distribution, pH and Osmolality of the formulations are summarized in Table 6.

TABLE 6
Characterization properties of LNP formulations containing
POX-grafted lipids for in vivo experiments.

| | Size | | | Osmolality | Zeta |
|---|---|---|---|---|---|
| Group | [d, nm] | PDI | pH | [mOsmol/kg] | (mV) |
| 1 | 67.8 | 0.21 | 5.29 | 375 | −1.3 |
| 2 | 78.1 | 0.21 | 5.37 | 367 | −16.6 |
| 3 | 86.6 | 0.24 | 5.30 | 362 | −21.6 |
| 4 | 74.0 | 0.16 | 5.18 | 370 | −0.1 |
| 5 | 72.7 | 0.16 | 5.28 | 378 | −1.6 |
| 6 | 79.1 | 0.19 | 5.10 | 363 | 3.0 |
| 7 | 81.8 | 0.21 | 5.20 | 374 | 3.7 |

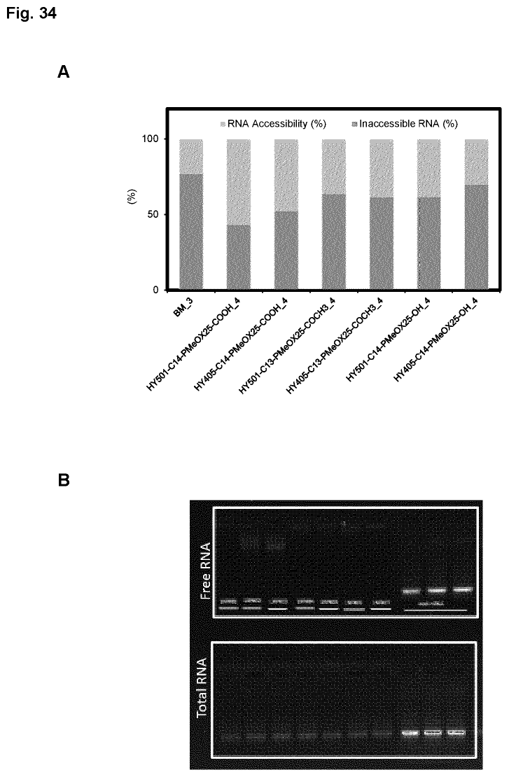

The accessibility of RNA and the amount of free RNA in the LNP formulations was measured by Ribogreen Assay and agarose gel electrophoresis methods, respectively. FIG. 34 present the RNA accessibility (A) and agarose gel electrophoresis (B) images of the formulations. As can be seen, high level of inaccessible RNA was measured for all formulations. On the other hand, the agarose gel electrophoresis images show no free RNA for all the formulations.

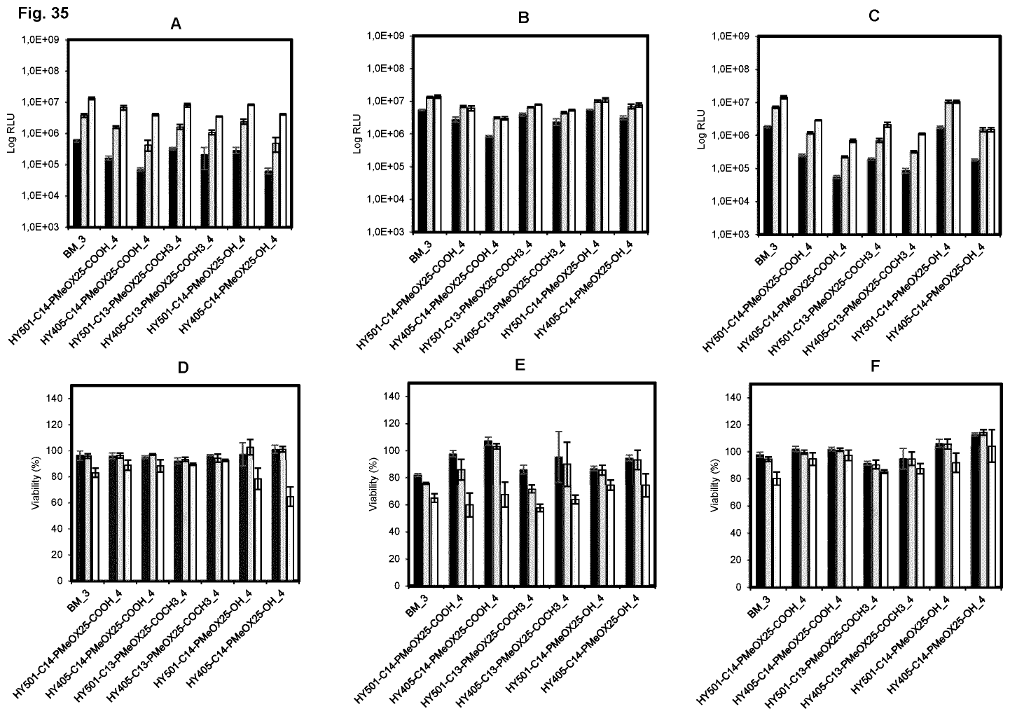

To investigate the LNP formulations potency, mRNA expression was evaluated in vitro using a skeletal muscle cell line (C2C12), a hepatocarcinoma cell line (HepG2) and a murine macrophage cell line (Raw cells). For this purpose, the LNP formulations comprising firefly luciferase-encoding mRNA were incubated with the cells at three dosages, 12.5, 25 and 50 ng per well and luciferase expression was assessed 24 h post-incubation. FIG. 35 shows that the luciferase expression (A-C) of formulations containing POX-grafted was almost in the same level of BM_3 formulation in C2C12 (A) and HepG2 (B) cell lines. No-to-low cytotoxicity (C2C12: D; HepG2: E; Raw cell: F) was observed for the LNP formulations in the tested doses in all the cell lines except for 50 ng dose in HepG2 cell line.

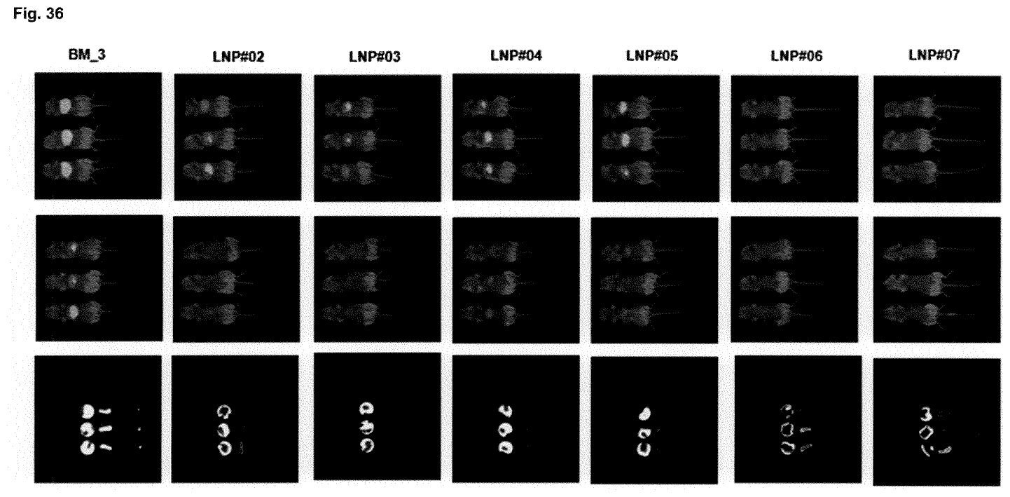

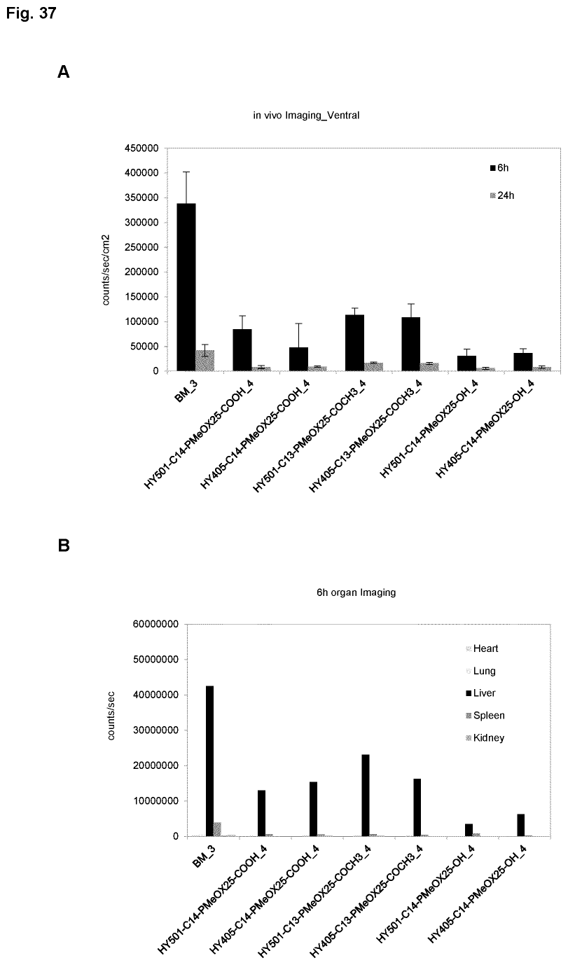

To investigate the functionality of the LNPs to mediate efficient and safe in vivo mRNA delivery, Balb/C mice were intravenously injected with 2 μg of luciferase-encoding mRNA containing LNPs. Whole animal and extracted organs were analyzed for bioluminescence (FIG. 36). As shown in FIGS. 36 and 37, 6 h post-injection for i.v. experiment, all LNPs mediated moderate to high mRNA expression in the liver region. Moreover, it can be observed that the level of mRNA expression of BM_3 formulation was higher than the HY501-POX (independent of POX functionality) formulations. This is most likely due to the ionizable lipid used in the BM_3 formulation (the ionizable lipid used in BM_3 was different from HY501). In addition, the comparison of the in vivo performance of the POX containing formulations show that the Luc signal in the liver for acetylated-POX containing HY501-LNP was higher than COOH—POX (at least 2-fold) and OH—POX (at least 4-fold) containing HY501-LNP. Moreover, the bioluminescence imaging of mice 24 h post-injection shows still some luciferase signal in the liver region.

### Example 9

The objective of this experiment was the investigation of C14-PMeOX45-NH2 lipid as stealth moiety for engineering mRNA nanoparticles using the LNP technology comprising HY501 as an ionizable lipid. The C14-PMeOX45-NH2 containing LNPs were prepared by mixing the RNA aqueous phase and the lipid organic phase using a microfluidic instrument. Different mole fraction of C14-PMeOX45-NH2 lipid (1-4%) were used for LNP formulations. The cationically ionizable lipid HY501, the helper lipid DSPC, and cholesterol at respective molar fractions 47.5:42.5-X: 10 was used, where x is the fraction of the C14-PMeOX45-NH2 lipid. The N/P ratio of 6 was selected for the experiments. Moreover, the physicochemical properties and biological performance of these C14-PMeOX45-NH2 containing formulations were compared with other PEG containing BM_2 formulation. A summary of all the formulations used in this study is given in Table 7 (for all formulations the RNA was mod. RNA luciferase, the RNA buffer was 100 mM CB pH 4.0, the dialysis buffer was PBS, and the final concentration was 0.05 mg/mL).

TABLE 7
Design of LNP formulations containing C14-PMeOX45-NH2.

| LNP | | |
|---|---|---|
| number | LNP name | Formulation composition/mol % |
| LNP#01 | BM_2 | — |
| LNP#02 | HY501-C14-PMeOX45-NH2_4 | HY501:CHOL:DSPC:C14-PMeOX45-NH2 (47.5:38.5:10:4) |
| LNP#03 | HY501-C14-PMeOX45-NH2_3 | HY501:CHOL:DSPC:C14-PMeOX45-NH2 (47.5:39.5:10:3) |
| LNP#04 | HY501-C14-PMeOX45-NH2_2 | HY501:CHOL:DSPC:C14-PMeOX45-NH2 (47.5:40.5:10:2) |
| LNP#05 | HY501-C14-PMeOX45-NH2_1 | HY501:CHOL:DSPC:C14-PMeOX45-NH2 (47.5:41.5:10:1) |

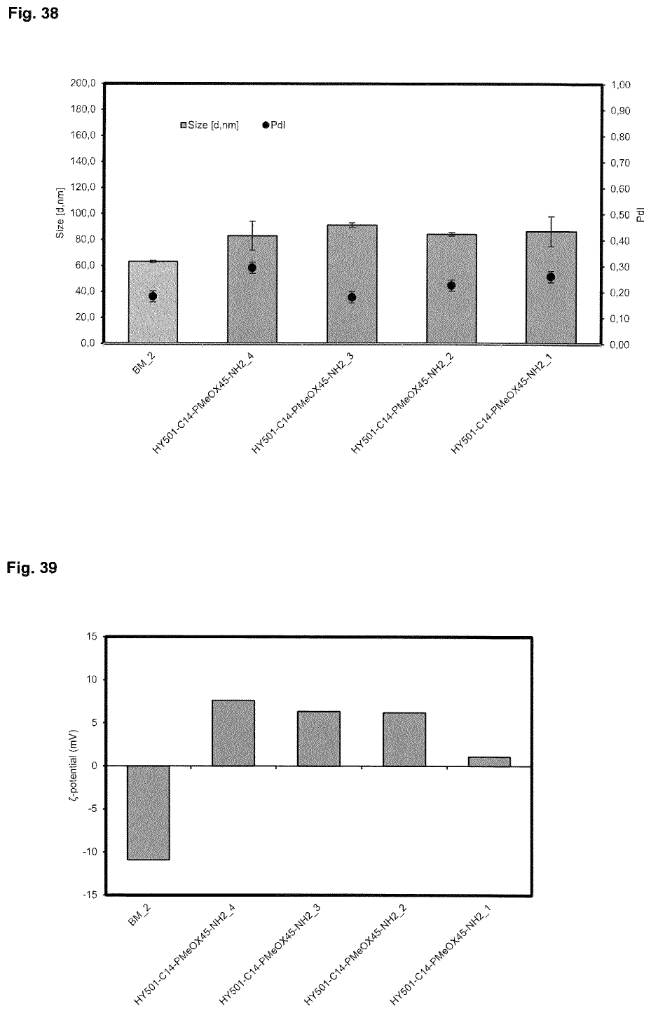

The evolution of size and size distribution of C14-PMeOX45-NH2 containing LNPs (based on HY-501) manufactured with different concentration of C14-PMeOX45-NH2 lipid in the formulation are shown in FIG. 38. As can be seen, the particle size in the range of 80-90 nm was observed for C14-PMeOX45-NH2 containing LNP formulations. On the other hand, all the formulations exhibited a PdI inferior to 0.3.

FIG. 39 shows the zeta potential of BM_2 and the C14-PMeOX45-NH2 containing LNPs measured in 0.1×PBS at pH 7. The results show that neutral to slightly positive surface charge was measured for C14-PMeOX45-NH2 containing LNPs. On the other hand, slight negative zeta potential was measured for BM_2 LNP formulation.

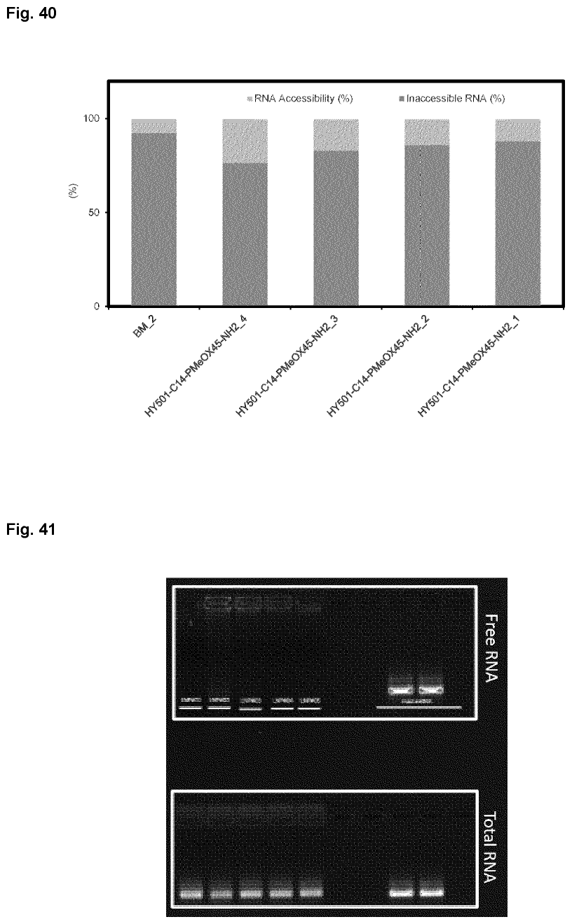

The accessibility of RNA and the amount of free RNA in the LNP formulations was measured by Ribogreen Assay and agarose gel electrophoresis methods, respectively. FIG. 40 presents the RNA accessibility of the formulations. As can be seen, high inaccessible RNA was obtained for all formulations and the accessibility of RNA in the formulations increased slightly with the concentration of C14-PMeOX45-NH2 lipid in the formulation from 1 to 4 mol %. On the other hand, the agarose gel electrophoresis images show almost no to negligible free RNA in the formulations (FIG. 41). These results from the agarose gel measurements show that virtually almost all of RNA is bound to the particles for all formulations.

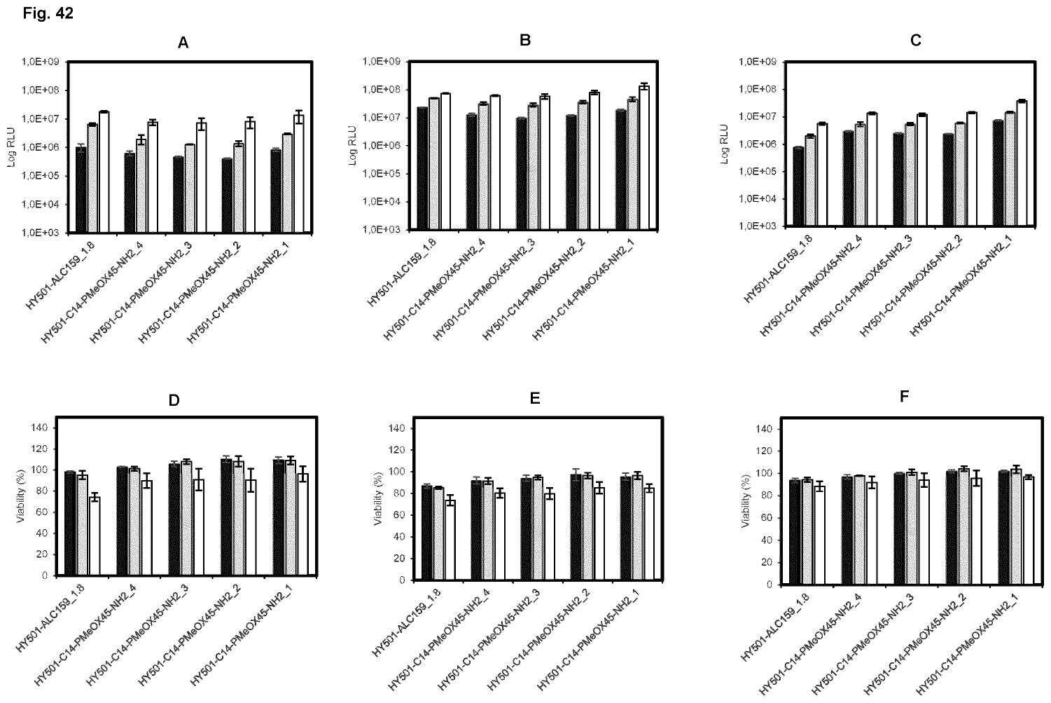

To investigate the C14-PMeOX45-NH2 LNP functionality, mRNA expression was evaluated in vitro using a skeletal muscle cell line (C2C12), a hepatocarcinoma cell line (HepG2) and a murine macrophage cell line (Raw cell). For this purpose, the LNP formulations comprising firefly luciferase-encoding mRNA were incubated with the cells at three dosages, 12.5, 25 and 50 ng per well and luciferase expression was assessed 24 h post-incubation. FIG. 42 shows that the luciferase expression (A-C) of formulation containing C14-PMeOX45-NH2 was similar or slightly higher than the BM_2 formulations in HepG2 and RAW cell lines in all the tested RNA doses (C2C12: A; HepG2: B; Raw cell: C; black bars: 12.5 ng; grey bars: 25 ng; white bars: 50 ng). Moreover, the highest performance was observed for the formulations containing 1-2 mol % of C14-PMeOX45-NH2 in HepG2 and RAW cell lines and the performance of the formulations decreased with increasing stealth lipid concentration. On the other hand, no-to-low cytotoxicity (C2C12: D; HepG2: E; Raw cell: F) was observed for the LNP formulations in the tested doses in all the cell lines.

### Example 10

The objective of this experiment was the investigation of C14-PMeOX45-COOH lipid as stealth moiety for engineering mRNA nanoparticles using the LNP technology comprising HY501 as an ionizable lipid. The C14-PMeOX45-COOH containing LNPs were prepared by mixing the RNA aqueous phase and the lipid organic phase using a microfluidic instrument. Different mole fraction of C14-PMeOX45-COOH lipid (1-5%) were used for LNP formulations. The ionizable lipid HY501, the helper lipid DSPC, and cholesterol at respective molar fractions 47.5:42.5-X: 10 was used, where x is the fraction of the C14-PMeOX45-COOH lipid. The N/P ratio of 6 was selected for the experiments. Moreover, the physicochemical properties and biological performance of these C14-PMeOX45-COOH containing formulations were compared with other PEG containing BM_2 formulation. A summary of all formulations used in this study is given in Table 8 (for all formulations the RNA was mod. RNA luciferase, the RNA buffer was 100 mM CB pH 4.0, the dialysis buffer was PBS, and the final concentration was 0.05 mg/mL).

TABLE 8
Design of LNP formulations containing C14-PMeOX45-COOH.

| LNP | | |
|---|---|---|
| number | LNP name | Formulation composition/mol % |
| LNP#01 | BM_2 | — |
| LNP#02 | HY501-C14-PMeOX45-COOH_5 | HY501:CHOL:DSPC:C14-PMeOX45-COOH (47.5:37.5:10:5) |
| LNP#03 | HY501-C14-PMeOX45- COOH_4 | HY501:CHOL:DSPC:C14-PMeOX45-COOH (47.5:38.5:10:4) |
| LNP#04 | HY501-C14-PMeOX45- COOH_3 | HY501:CHOL:DSPC:C14-PMeOX45-COOH (47.5:39.5:10:3) |
| LNP#05 | HY501-C14-PMeOX45- COOH_2 | HY501:CHOL:DSPC:C14-PMeOX45-COOH (47.5:40.5:10:2) |
| LNP#06 | HY501-C14-PMeOX45- COOH_1 | HY501:CHOL:DSPC:C14-PMeOX45-COOH (47.5:41.5:10:1) |

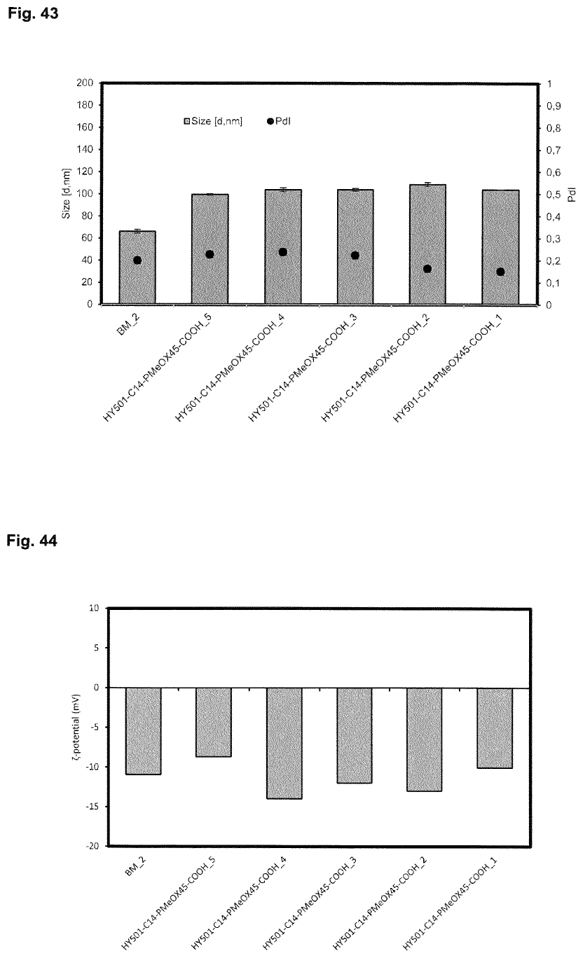

The evolution of size and size distribution of C14-PMeOX45-COOH containing LNPs (based on HY-501) manufactured with different concentration of C14-PMeOX45-COOH lipid in the formulation are shown in FIG. 43. As can be seen, the particle size in the range of 100-110 nm was observed for the C14-PMeOX45-COOH containing LNP formulations. On the other hand, the PDI of the LNP formulations increased slightly with increasing C14-PMeOX45-COOH concentration in the formulation. Moreover, all formulations exhibited a PDI inferior to 0.3.

FIG. 44 shows the zeta potential of BM_2 and the C14-PMeOX45-COOH containing LNPs measured in 0.1×PBS at pH 7. As can be seen, high negative surface charge was measured for all C14-PMeOX45-COOH containing LNPs.

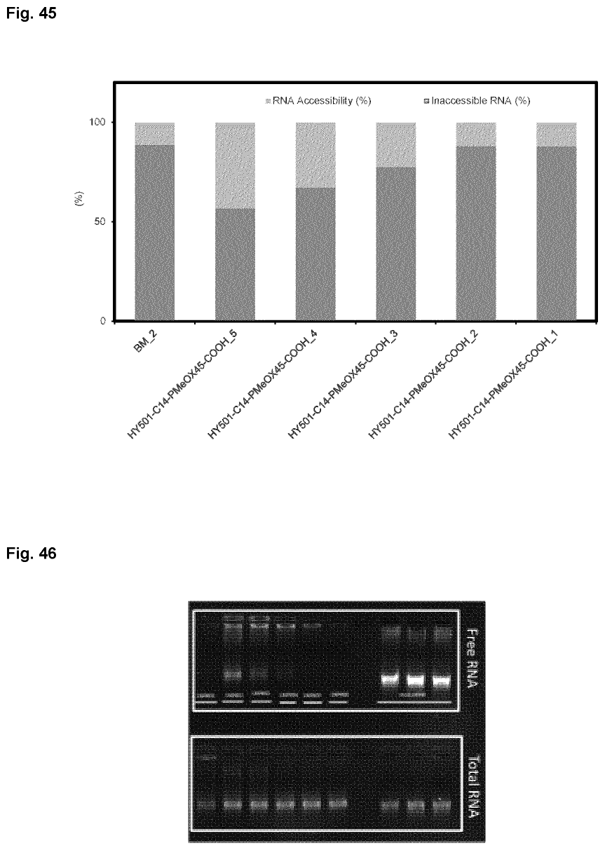

The accessibility of RNA and the amount of free RNA in the LNP formulations was measured by Ribogreen Assay and Agarose gel electrophoresis methods, respectively. FIG. 45 presents the RNA accessibility of the formulations. As can be seen, high level of RNA inaccessibility was observed for all formulations and the amount of accessible RNA increased with increasing stealth moiety concentration. On the other hand, the agarose gel electrophoresis images show almost no to negligible free RNA in the formulations containing 1-3 mol % C14-PMeOX45-COOH (FIG. 46). However, higher amount of free RNA was observed for the formulations containing 4-5 mol % C14-PMeOX45-COOH. These results from the agarose gel measurements show that virtually almost all of RNA is bound to the particles for the formulations containing 1-3 mol % C14-PMeOX45-COOH.

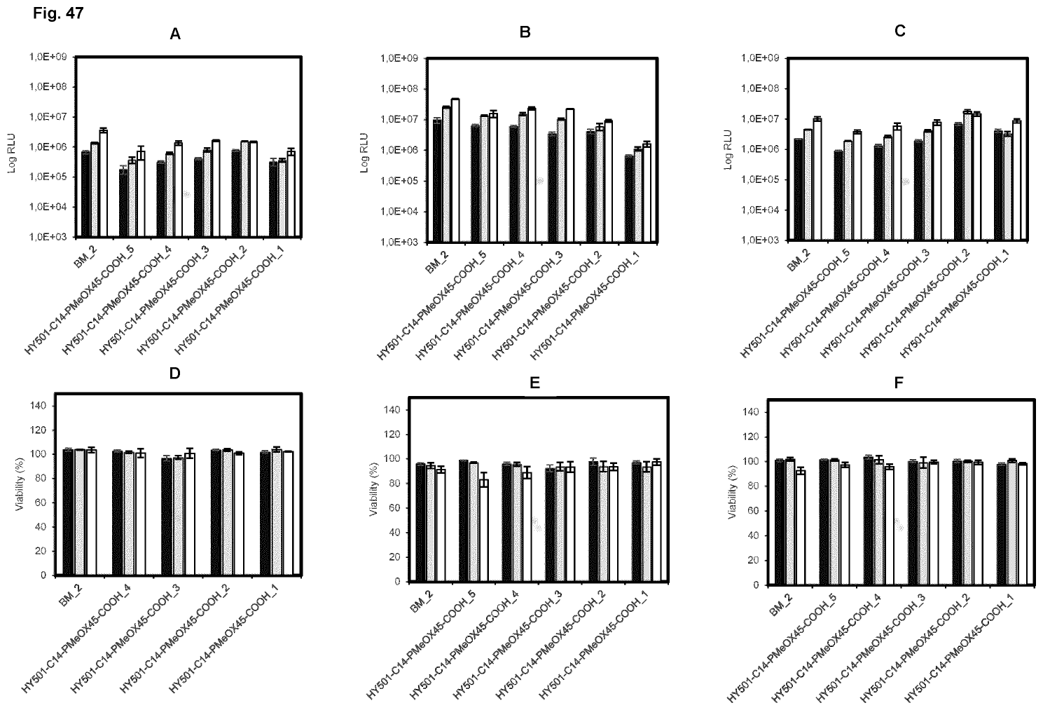

To investigate the C14-PMeOX45-COOH LNP functionality, mRNA expression was evaluated in vitro using a skeletal muscle cell line (C2C12), a hepatocarcinoma cell line (HepG2) and a murine macrophage cell line (Raw cell). For this purpose, the LNP formulations comprising firefly luciferase-encoding mRNA were incubated with the cells at three dosages, 12.5, 25 and 50 ng per well and luciferase expression was assessed 24 h post-incubation. FIG. 47 shows that the luciferase expression (A-C) of formulations containing C14-PMeOX45-COOH was similar or slightly lower (in C2C12 and HepG2 cell lines) than the BM_2 formulations in all the tested RNA doses (C2C12: A; HepG2: B; Raw cell: C). On the other hand, no-to-low cytotoxicity (C2C12: D; HepG2: E; Raw cell: F) was observed for the LNP formulations in the tested doses in all the cell lines.

### Example 11

The objective of this experiment was the investigation of C14-PMeOX45-OH lipid as stealth moiety for engineering mRNA nanoparticles using the LNP technology comprising HY501 as an ionizable lipid. The C14-PMeOX45-OH containing LNPs were prepared by mixing the RNA aqueous phase and the lipid organic phase using a microfluidic instrument. Different mole fraction of C14-PMeOX45-OH lipid (2-5%) were used for LNP formulations. The ionizable lipid HY501, the helper lipid DSPC, and cholesterol at respective molar fractions 47.5:42.5-X: 10 was used, where x is the fraction of the C14-PMeOX45-OH lipid. The N/P ratio of 6 was selected for the experiments. Moreover, the physicochemical properties and biological performance of these C14-PMeOX45-OH containing formulations were compared with other PEG containing BM_2 formulation. A summary of all the formulations used in this study is given in Table 9 (for all formulations the RNA was mod. RNA luciferase, the RNA buffer was 100 mM CB pH 4.0, the dialysis buffer was PBS, and the final concentration was 0.05 mg/mL).

TABLE 9
Design of LNP formulations containing C14-PMeOX45-OH.

| LNP | | |
|---|---|---|
| number | LNP name | Formulation composition/mol % |
| LNP#01 | BM_2 | — |
| LNP#02 | HY501-C14-PMeOX45-OH_5 | HY501:CHOL:DSPC:C14-PMeOX45-OH (47.5:37.5:10:5) |
| LNP#03 | HY501-C14-PMeOX45-OH_4 | HY501:CHOL:DSPC:C14-PMeOX45-OH (47.5:38.5:10:4) |
| LNP#04 | HY501-C14-PMeOX45-OH_3 | HY501:CHOL:DSPC:C14-PMeOX45-OH (47.5:39.5:10:3) |
| LNP#05 | HY501-C14-PMeOX45-OH_2 | HY501:CHOL:DSPC:C14-PMeOX45-OH (47.5:40.5:10:2) |

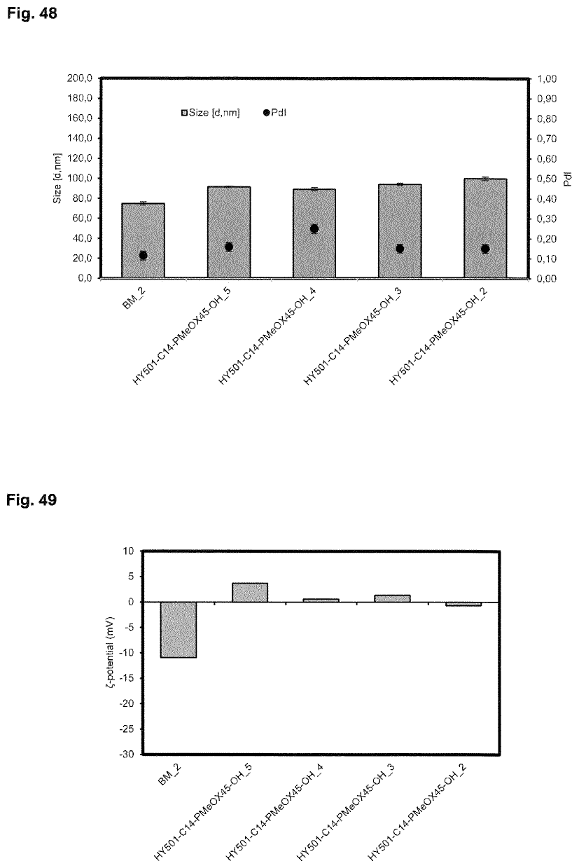

The evolution of size and size distribution of C14-PMeOX45-OH containing LNPs (based on HY-501) manufactured with different concentration of C14-PMeOX45-OH lipid in the formulation are shown in FIG. 48. As can be seen, the particle size of the formulations is in 90-100 nm range for the formulations containing 2-5 mol % of C14-PMeOX45-OH lipid. On the other hand, all the formulations exhibited a PdI inferior to 0.3.

FIG. 49 shows the zeta potential of BM_2 and the C14-PMeOX45-OH containing LNPs measured in 0.1×PBS at pH 7. As can be seen, neutral surface charge was measured for all C14-PMeOX45-OH containing LNPs.

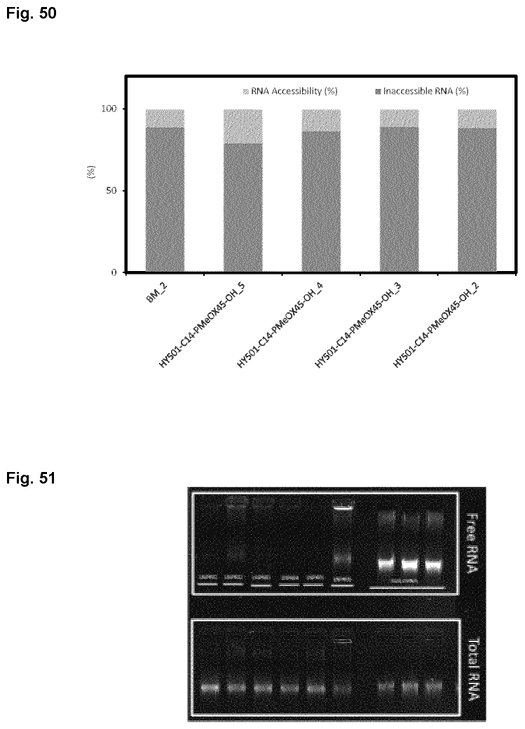

The accessibility of RNA and the amount of free RNA in the LNP formulations was measured by Ribogreen Assay and agarose gel electrophoresis methods, respectively. FIG. 50 presents the RNA accessibility of the formulations. As can be seen, high level of RNA inaccessibility was observed for all tested formulations. On the other hand, the agarose gel electrophoresis images show almost no to negligible free RNA in all tested formulations (FIG. 51). These results from the agarose gel measurements show that virtually almost all of RNA is bound to the particles for formulations containing 2-5 mol % C14-PMeOX45-OH.

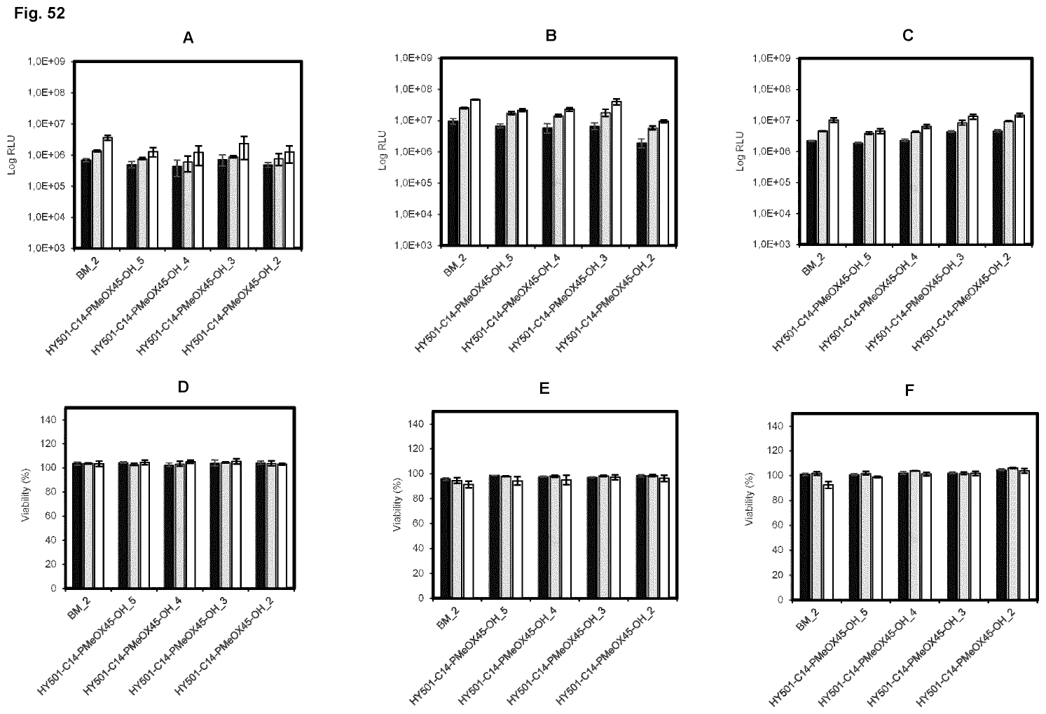

To investigate the C14-PMeOX45-OH LNP functionality, mRNA expression was evaluated in vitro using a skeletal muscle cell line (C2C12), a hepatocarcinoma cell line (HepG2) and a murine macrophage cell line (Raw cell). For this purpose, the LNP formulations comprising firefly luciferase-encoding mRNA were incubated with the cells at three dosages, 12.5, 25 and 50 ng per well and luciferase expression was assessed 24 h post-incubation. FIG. 52 shows that the luciferase expression (A-C) of formulation containing C14-PMeOX45-OH was similar or slightly higher than the BM_2 formulations in all the tested cell lines (C2C12: A; HepG2: B; Raw cell: C). Moreover, the performance of the formulations decreased slightly with increasing stealth lipid concentration. On the other hand, no-to-low cytotoxicity (C2C12: D; HepG2: E; Raw cell: F) was observed for the LNP formulations in the tested doses in all the cell lines.

### Example 12

The objective of this experiment was the investigation of C14-PMeOX45-NH-AC lipid as stealth moiety for engineering mRNA nanoparticles using the LNP technology comprising HY501 as an ionizable lipid. In this study the C14-PMeOX45-NH-AC lipid has been synthesized by Chemistry team in Formulation Discovery and Testing Unit as shown in the following scheme and used as stealth moiety in LNP formulations.

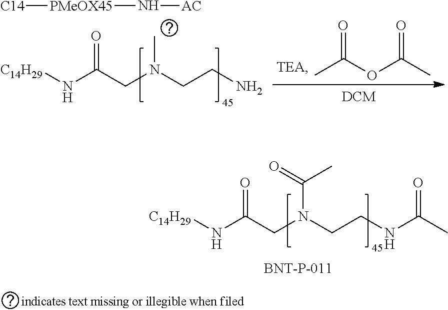

The C14-PMeOX45-NH-AC containing LNPs were prepared by mixing the RNA aqueous phase and the lipid organic phase using a microfluidic instrument. Different mole fraction of C14-PMeOX45-NH-AC lipid (1-4%) were used for LNP formulations. The ionizable lipid HY501, the helper lipid DSPC, and cholesterol at respective molar fractions 47.5:42.5-X: 10 was used, where x is the fraction of the C14-PMeOX45-NH-AC lipid. The N/P ratio of 6 was selected for the experiments. Moreover, the physicochemical properties and biological performance of these C14-PMeOX45-NH-AC containing formulations were compared with other PEG containing BM_2 formulation. A summary of all the formulations used in this study is given in Table 10 (for all formulations the RNA was mod. RNA luciferase, the RNA buffer was 100 mM CB pH 4.0, the dialysis buffer was PBS, and the final concentration was 0.05 mg/mL).

TABLE 10
Design of LNP formulations containing C14-PMeOX45-NH-AC.

| LNP | | |
|---|---|---|
| number | LNP name | Formulation composition/mol % |
| LNP#01 | BM_2 | — |
| LNP#02 | HY501-C14-PMeOX45-NH-AC_4 | HY501:CHOL:DSPC:C14-PMeOX45-NH-AC (47.5:38.5:10:4) |
| LNP#03 | HY501-C14-PMeOX45-NH-AC_3 | HY501:CHOL:DSPC:C14-PMeOX45-NH-AC (47.5:39.5:10:3) |
| LNP#04 | HY501-C14-PMeOX45-NH-AC_2 | HY501:CHOL:DSPC:C14-PMeOX45-NH-AC (47.5:40.5:10:2) |
| LNP#05 | HY501-C14-PMeOX45-NH-AC_1 | HY501:CHOL:DSPC:C14-PMeOX45-NH-AC (47.5:41.5:10:1) |

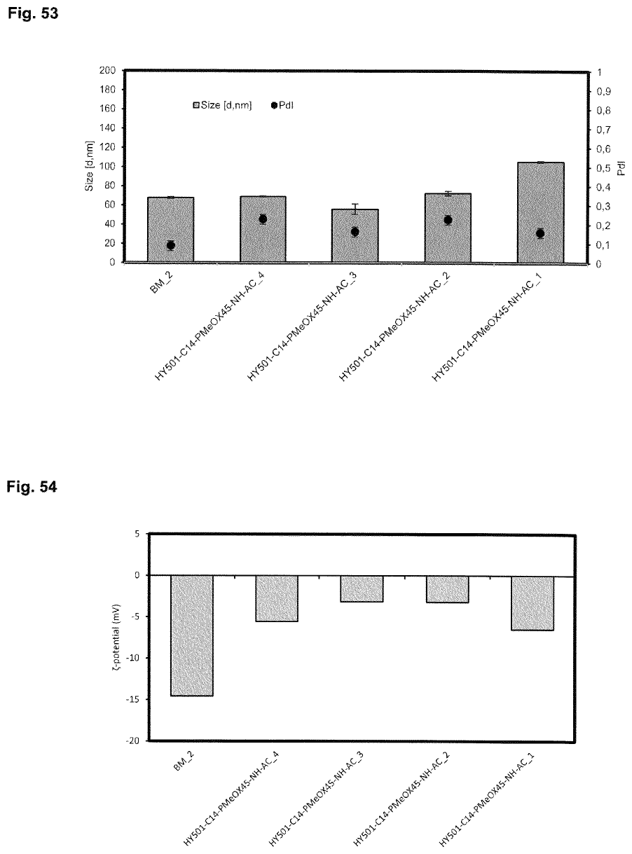

The evolution of size and size distribution of C14-PMeOX45-NH-AC containing LNPs (based on HY-501) manufactured with different concentration of C14-PMeOX45-NH-AC lipid in the formulation are shown in FIG. 53. As can be seen, the particle size of the formulations is in 55-105 nm range for the formulations containing 1-4 mol % of C14-PMeOX45-NH-AC lipid. On the other hand, all the formulations exhibited a PdI inferior to 0.25.

FIG. 54 shows the zeta potential of BM_2 and the C14-PMeOX45-NH-AC containing LNPs measured in 0.1×PBS at pH 7. As can be seen, slightly negative charge was measured for all C14-PMeOX45-NH-AC containing LNPs.

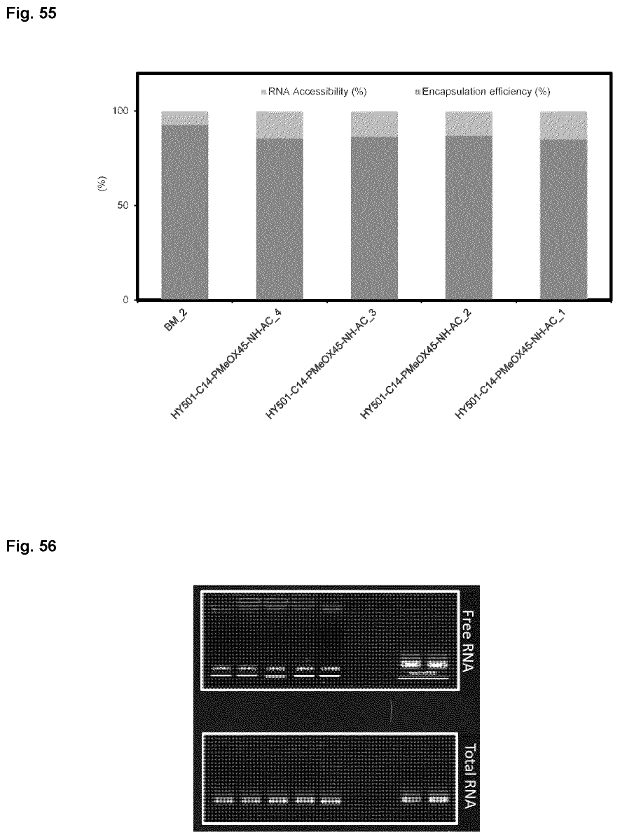

The accessibility of RNA and the amount of free RNA in the LNP formulations was measured by Ribogreen Assay and agarose gel electrophoresis methods, respectively. FIG. 55 presents the RNA accessibility of the formulations. As can be seen, high level of RNA inaccessibility was observed for all tested formulations. On the other hand, the agarose gel electrophoresis images show almost no to negligible free RNA in all tested formulations (FIG. 56). These results from the agarose gel measurements show that virtually almost all of RNA is bound to the particles for formulations containing 1-4 mol % C14-PMeOX45-NH-AC.

### Example 13

The objective of this experiment was the investigation of VE-PMeOX50-CH3 lipid as stealth moiety for engineering mRNA nanoparticles using the LNP technology comprising HY501 as an ionizable lipid. In this experiment the VE-PMeOX50-CH3 has been synthesized by Chemistry team in Formulation Discovery and Testing Unit as shown in the following scheme and used as stealth moiety in LNP formulations.

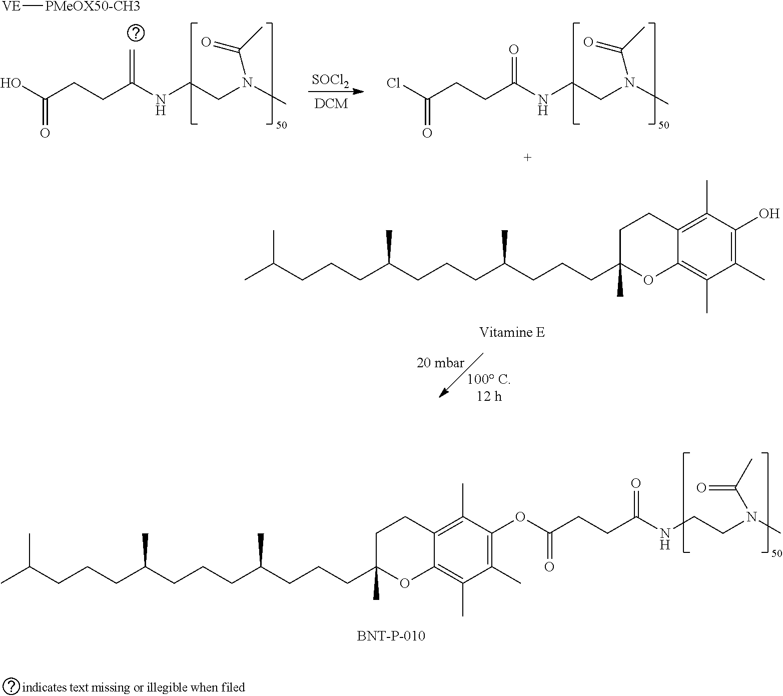

The VE-PMeOX50-CH3 containing LNPs were prepared by mixing the RNA aqueous phase and the lipid organic phase using a microfluidic instrument. Different mole fraction of VE-PMeOX50-CH3 lipid (1-4%) were used for LNP formulations. The ionizable lipid HY501, the helper lipid DSPC, and cholesterol at respective molar fractions 47.5:42.5-X: 10 was used, where x is the fraction of the VE-PMeOX50-CH3 lipid. N/P ratio of 6 was selected for the experiments. Moreover, the physicochemical properties and biological performance of these VE-PMeOX50-CH3 containing formulations were compared with other PEG containing BM_2 formulation. A summary of all the formulations used in this study is given in Table 11 (for all formulations the RNA was mod. RNA luciferase, the RNA buffer was 50 mM AB pH 4.0, the dialysis buffer was PBS, and the final concentration was 0.05 mg/mL).

TABLE 11
Design of LNP formulations containing VE-PMeOX50-CH3.

| LNP | | |
|---|---|---|
| number | LNP name | Formulation composition/mol % |
| LNP#01 | BM_2 | — |
| LNP#02 | HY501-VE-PMeOX50-CH3_4 | HY501:CHOL:DSPC:VE-PMeOX50-CH3 (47.5:38.5:10:4) |
| LNP#03 | HY501--VE-PMeOX50-CH3_3 | HY501:CHOL:DSPC:VE-PMeOX50-CH3 (47.5:39.5:10:3) |
| LNP#04 | HY501-VE-PMeOX50-CH3_2 | HY501:CHOL:DSPC:VE-PMeOX50-CH3 (47.5:40.5:10:2) |
| LNP#05 | HY501-VE-PMeOX50-CH3_1 | HY501:CHOL:DSPC:VE-PMeOX50-CH3 (47.5:41.5:10:1) |

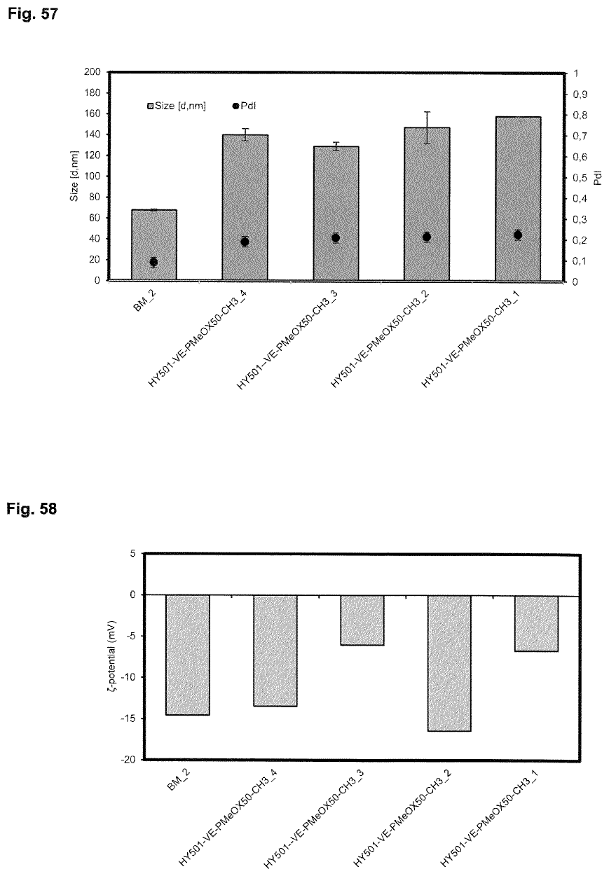

The evolution of size and size distribution of VE-PMeOX50-CH3 containing LNPs (based on HY-501) manufactured with different concentration of VE-PMeOX50-CH3 lipid in the formulation are shown in FIG. 57. As can be seen, the particle size of the formulations is in 130-160 nm range for the formulations containing 1-4 mol % of VE-PMeOX50-CH3 lipid. On the other hand, all the formulations exhibited a PdI inferior to 0.25.

FIG. 58 shows the zeta potential of BM_2 and the VE-PMeOX50-CH3 containing LNPs measured in 0.1×PBS at pH 7. As can be seen, slightly negative charge was measured for all VE-PMeOX50-CH3 containing LNPs.

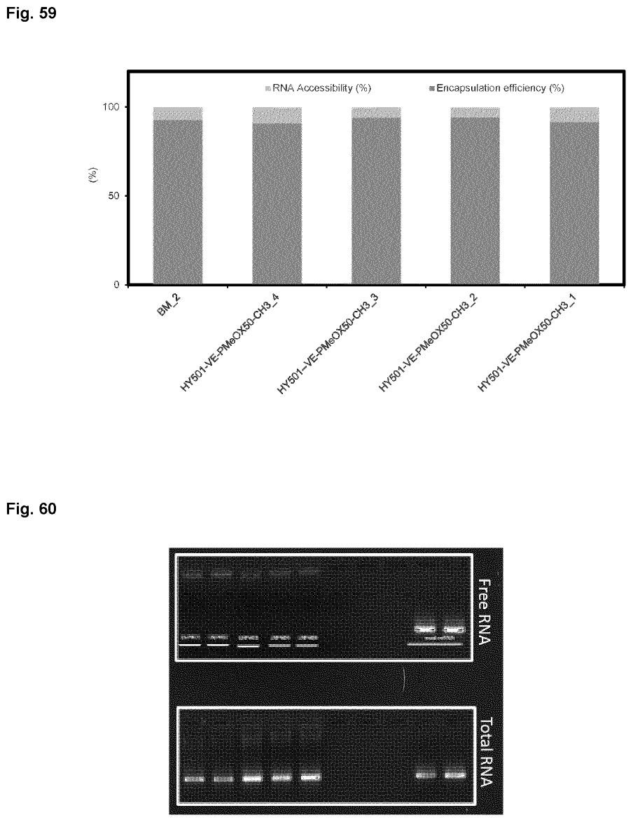

The accessibility of RNA and the amount of free RNA in the LNP formulations was measured by Ribogreen Assay and agarose gel electrophoresis methods, respectively. FIG. 59 presents the RNA accessibility of the formulations. As can be seen, high level of RNA inaccessibility was observed for all tested formulations. On the other hand, the agarose gel electrophoresis images show almost no to negligible free RNA in all tested formulations (FIG. 60). These results from the agarose gel measurements show that virtually almost all of RNA is bound to the particles for formulations containing 1-4 mol % VE-PMeOX50-CH3.

1. A composition comprising lipid nanoparticles (LNPs), wherein the LNPs comprise:
(i) RNA;
(ii) a cationic or cationically ionizable lipid; and
(iii) a conjugate of (a) a polyoxazoline (POX) and/or polyoxazine (POZ) polymer and (b) one or more hydrophobic chains,
wherein the conjugate has the following general formula (II) or (II′): 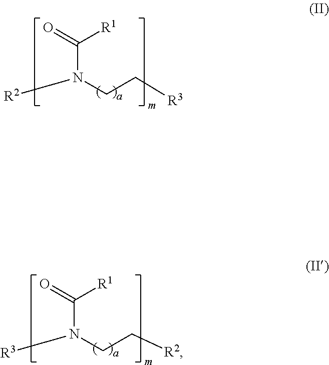
wherein:
a is an integer between 1 and 2;
R¹ is alkyl, in particular C₁₋₃ alkyl, such as methyl, ethyl, iso-propyl, or n-propyl, and is independently selected for each repeating unit;
m is 2 to 200;
R² is R⁴ or -L¹ (R⁴)ₚ, wherein R⁴ is a hydrocarbyl group; L¹ is a linker; and p is 1; and
R³ is selected from the group consisting of H, C₁₋₆ alkyl, C₂₋₆ alkynyl, —OR²⁰, —SR²⁰, halogen, ₃, —OC(O)R²¹, —C(O)R²¹—NR²²R²³, —COOH, —C(O)NR²²R²³, —NR²²C(O)R²¹, ₁₋₆ alkyl group is optionally substituted with one or more substituents independently selected from the group consisting of —OH, SH, halogen, —CN, —N₃, C₂₋₆ alkynyl, —COOH, —COOCH₃, —NR²²R²³, ²²R²³, —NR²²C(O)R²¹, a sugar, an amino acid, a peptide, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; R²¹ is selected from the group consisting of C₁₋₆ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, a sugar, an amino acid, a peptide, and a member of a targeting pair; and each of R²² and R²³ is independently selected from the group consisting of H, alkyl, alkenyl, alkynyl, cycloalkyl, aryl, heteroaryl, and heterocyclyl, or R²² and R²³ may join together with the nitrogen atom to which they are attached to form a heterocyclyl group, wherein each of the alkyl, alkenyl, alkynyl, cycloalkyl, aryl, heteroaryl, and heterocyclyl groups is optionally substituted with one or more substituents independently selected from the group consisting of —OH, SH, halogen, ₃, C₂₋₆ alkynyl, —COOH, —NH₂, —NH(C₁₋₃ alkyl), —N(C₁₋₃ alkyl)₂, a sugar, an amino acid, a peptide, and a member of a targeting pair.

2.-4. (canceled)

5. The composition of claim 1, wherein
(i) R¹ is methyl or ethyl, and/or
(ii) m is 2 to 180.

6. (canceled)

7. The composition of claim 1, wherein L¹ comprises at least one alkylene moiety substituted with at least one monovalent functional moiety and/or linked, at the end by which the alkylene group is attached to R⁴, to a divalent functional moiety, wherein each monovalent functional moiety is independently selected from hydroxy, ether, halogen, cyano, azido, nitro, amino, ammonium, ester, carboxyl, thiol (sulfanyl), disulfanyl, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imide, and amide moieties; and/or each divalent functional moiety is independently selected from ether, amino, ester, sulfide, disulfide, sulfoxide, sulfone, sulfite, sulfate, phosphate, sulfinamide, sulfonamide, sulfamate, diselenide, sulfurous diamide, sulfuric diamide, urea, thiourea, carbonyl, thiocarbonyl, orthoester, thioate, dithioate, imidate, imino, imidothioate, thionylamido, carbonate, carbonothioate, carbonodithioate, carbonotrithioate, guanidino (imidamido), carbamimidate, carbonimidate, carbamate, carbamodithioate, carbonodithioimidate, carbamimidothioate, carbamothioate, carbonimidothioate, acylhydrazone, hydrazine, oxime, acetal, hemiacetal, ketal, hemiketal, imine, imide, and amide moieties.

8. The composition of claim 1, wherein
(i) L¹ is selected from the group consisting of [*—NHC(O)]ₚ(C₁₋₆-alkylene)-, [*—C(O)NH]ₚ(C₁₋₆-alkylene)-, [*—C(O)O]ₚ(C₁₋₆-alkylene)-, [*—OC(O)]ₚ(C₁₋₆-alkylene)-, [*—S]ₚ(C₁₋₆-alkylene)-, [*—SS]ₚ(C₁₋₆-alkylene)-, [*—S(O)₂]ₚ(C₁₋₆-alkylene)-, [(*—O)ᵣC(OR²⁵)₃₋ᵣ](C₁₋₆-alkylene)-, [*—C(OR²⁵)₂O]ₚ(C₁₋₆-alkylene)-, [*—C(R²⁵)(═N—N(R²⁶)C(O)—)]ₚ(C₁₋₆-alkylene)-, [*—C(O)(N(R²⁶)—N═)C(R²⁵)—]ₚ(C₁₋₆-alkylene)-, [*═C(═N—N(R²⁶)C(O)(R²⁵))]ₚ(C₁₋₆-alkylene)-, [*—N(R²⁶)N(R²⁶)]ₚ(C₁₋₆-alkylene)-, [*═C(═N(OH))]ₚ(C₁₋₆-alkylene)-, and [*—OC(R²⁵)(R²⁶)O]ₚ(C₁₋₆-alkylene)-, wherein * represents the attachment point to R⁴; C₁₋₆-alkylene is bivalent; R²⁵ is selected from the group consisting of C₁₋₆ alkyl, aryl, and aryl(C₁₋₆ alkyl); R²⁶ is selected from the group consisting of H, C₁₋₆ alkyl, aryl, and aryl(C₁₋₆ alkyl); and r is an integer between 1 and 2;
(ii) L¹ is selected from the group consisting of *—NHC(O)—(CH₂)—, *—NHC(O)—(CH₂)₂—, *—C(O)NH—(CH₂)—, *—C(O)NH—(CH₂)₂—, —(CH₂)—CH(OC(O)—*)(CH₂OC(O)—*), —(CH₂)—CH(S—*)₂, —(CH₂)—CH(S—*)—CH₂(S—*), *—S—(CH₂)₃—, *—S(O)₂—(CH₂)₂—, and *—OC(O)—(CH₂)—, wherein * represents the attachment point to R⁴;
(iii) L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₂—, and *—OC(O)—(CH₂)— (preferably L¹ is selected from the group consisting of *—NHC(O)—(CH₂)—, *—NHC(O)—(CH₂)₂—, *—C(O)NH—(CH₂)—, and *—C(O)NH—(CH₂)₂—, such as *—NHC(O)—(CH₂)— or *—NHC(O)—(CH₂)₂—), wherein * represents the attachment point to R⁴;
(iv) L¹ is selected from the group consisting of [*—C(O)O], (C₁₋₆-alkylene)-OP(O)₂O(C₁₋₆-alkylene) NHC(O)—(C₁₋₆-alkylene)-, *—OC(O)]ₚ(C₁₋₆-alkylene)-OP(O)₂O(C₁₋₆-alkylene) NHC(O)—(C₁₋₆-alkylene)-, and [*—C(O)](C₁₋₆-alkylene)-C(O)—, wherein * represents the attachment point to R⁴; and C₁₋₆-alkylene is bivalent;
(v) *—C(O)(C₂₋₃-alkylene)-C(O)—, wherein * represents the attachment point to R⁴; and/or
(vi) R² is R⁴—C(O)(CH₂)₂—C(O)—.

9.-13. (canceled)

14. The composition of claim 1, wherein the conjugate has the general formula (II′) and
(i) L¹ is selected from the group consisting of [*—Z]ₚ(C₁₋₆-alkylene)-Z—, *—Z—(C₃₋₈-cycloalkylene)-Z—, *—Z—(C₃₋₈-cycloalkenylene)-Z—, (*═N)(C₁₋₆-alkylene)-Z—, *—Z—(C₁₋₆-alkylene)-, and *—Z—, wherein * represents the attachment point to R⁴; C₁₋₆-alkylene is either bivalent (if p is 1) or trivalent (if p is 2); each of the C₃₋₈-cycloalkylene and C₃₋₈-cycloalkenylene groups is optionally substituted with 1, 2, 3, or 4 substituents independently selected from the group consisting of —OH, ═O, —SH, halogen, —CN, —N₃, and C₁₋₃-alkyl; and each Z is independently selected from the group consisting of —OP(O)₂O(C₁₋₃-alkylene) NH—, —NH(C₁₋₃-alkylene) OP(O)₂O—, —C(O)NH—, —NHC(O)—, OC(O)NH—, —NHC(O)O—, —O—, —C(O)O—, —OC(O)—, —S—, —S(O)₂—, and —NR²²—;
(ii) L¹ is [*—C(O)], (C₁₋₆-alkylene)-C(O)NH—, wherein * represents the attachment point to R⁴; and C₁₋₆-alkylene is bivalent;
(iii) L¹ is *—C(O)(C₂₋₃-alkylene)-C(O)NH—, wherein * represents the attachment point to R⁴; and/or
(iv) R² is R⁴—C(O)(CH₂) ¿C(O)NH— or R⁴NH—.

15.-17. (canceled)

18. The composition of claim 1, wherein R⁴ is
(i) a non-cyclic, preferably straight hydrocarbyl group;
(ii) a hydrocarbyl group having at least 8 carbon atoms;
(iii) a non-cyclic, straight hydrocarbyl group having at least 10 carbon atoms; or
(iv) a tocopherol moiety.

19.-20. (canceled)

21. The composition of claim 1, wherein
(i) R³ is selected from the group consisting of H, C₁₋₃ alkyl, —OR²⁰, —N₃, C₂₋₆ alkynyl, —OC(O)R²¹, —C(O)R²¹, —NR²²R²³, —COOH, —C(O)NR²²R²³, —NR²²C(O)R²¹, and a member of a targeting pair, wherein the C₁₋₃ alkyl group is optionally substituted with one or more substituents independently selected from the group consisting of —OH, SH, halogen, —CN, —N₃, C₂₋₆ alkynyl, —COOH, —COOCH₃, —NR²²R²³, —C(O)NR²²R²³, —NR²²C(O)R²¹, and a member of a targeting pair; R²⁰ is selected from the group consisting of H, C₁₋₃ alkyl and 3- to 6-membered heterocyclyl, wherein each of the C₁₋₃ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; R²¹ is selected from the group consisting of C₁₋₃ alkyl and 3- to 6-membered heterocyclyl, wherein each of the C₁₋₆ alkyl and 3- to 6-membered heterocyclyl groups is optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₆ alkyl, C₂₋₆ alkenyl, and C₂₋₆ alkynyl, or R²² and R²³ may join together with the nitrogen atom to which they are attached to form a heterocyclyl group, wherein each of the C₁₋₆ alkyl, C₂₋₆ alkenyl, and C₂₋₆ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair;
(ii) R³ is selected from the group consisting of H, C₁₋₃ alkyl, —OR²⁰, —N₃, C₂₋₆ alkynyl, —OC(O)R²¹, —C(O)R²¹, —NR²²R²³, —COOH, ²²R²³, —NR²²C(O)R²¹, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, ²²R²³, —NR²²C(O)R²¹, and a member of a targeting pair; R²⁰ is selected from the group consisting of H and C₁₋₃ alkyl; R²¹ is C₁₋₃ alkyl optionally substituted with one or more substituents independently selected from the group consisting of —OH, SH, halogen, —CN, —N₃, C₂₋₆ alkynyl, —COOH, —NR²²R²³, and a member of a targeting pair; and each of R²² and R²³ is independently selected from the group consisting of H, C₁₋₃ alkyl, C₂₋₃ alkenyl, and C₂₋₃ alkynyl, wherein each of the C₁₋₃ alkyl, C₂₋₃ alkenyl, and C₂₋₃ alkynyl groups is optionally substituted with one or more substituents independently selected from the group consisting of —OH, SH, halogen, —CN, —N₃, C₂₋₆ alkynyl, —COOH, —NH₂, —NH(C₁₋₃ alkyl), —N(C₁₋₃ alkyl)₂, and a member of a targeting pair, or R²² and R²³ may join together with the nitrogen atom to which they are attached to form a 5- or 6-membered heterocyclyl group; or
(iii) R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), ₂)₂COOH, —N(CH₂CH₃)C(O)(CH₂)₂COOH, —N(CH₂CH₃)C(O) CH₃, —C(O)NH₂, ₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, ₃, —N(CH₃)₂, —C(O)NH₂, —C(O)NHCH₃, —C(O)NH(CH₂)₂NH₂, and a member of a targeting pair.

22.-23. (canceled)

24. The composition of claim 1, wherein the conjugate has the following general formula (III) or (III′): 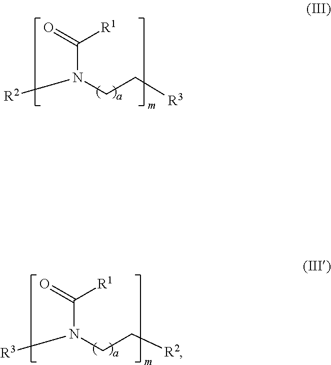
wherein:
(A)
a is an integer between 1 and 2;
R¹ is methyl or ethyl and is independently selected for each repeating unit;
m is 10 to 100;
R², for formula (III), is selected from the group consisting of -L¹R⁴, wherein R⁴ is a straight hydrocarbyl group having at least 10 carbon atoms; and L¹ is selected from the group consisting of *—NHC(O)—(CH₂)—, *—NHC(O)—(CH₂)₂—, *—C(O)NH—(CH₂)—, *—C(O)NH—(CH₂)₂—, *—S—(CH₂)₃—, *—S(O)₂—(CH₂)₃—, and *—OC(O)—(CH₂) wherein * represents the attachment point to R⁴; or R², for formula (III′), is selected from the group consisting of R⁴Z(C₁₋₃-alkylene)-Z—, R⁴Z—(C₃₋₆-cycloalkenylene)-Z—, and R⁴Z—, wherein the C₃₋₆-cycloalkenylene group is optionally substituted with 1, 2, 3, or 4 substituents independently selected from the group consisting of —OH, ═O, —SH, halogen, —CN, —N₃, and C₁₋₃-alkyl; and each Z is independently selected from the group consisting of —OP(O)₂O(CH₂)₂NH—, —NH(CH₂)₂OP(O)₂O—, —C(O)NH—, —NHC(O)—, —OC(O)NH—, —NHC(O)O—, —O—, —C(O)O—, ₂—, and —NH—; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, ₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, —C(O)NH(CH₂)₂NH₂, and a member of a targeting pair; or
(B)
a is an integer between 1 and 2;
R¹ is methyl or ethyl and is independently selected for each repeating unit;
m is 10 to 100;
R², for formula (III), is R⁴—C(O)(CH₂)₂—C(O)—, wherein R⁴ is a straight hydrocarbyl group having at least 10 carbon atoms; or R², for formula (III′), is R⁴—C(O)(CH₂)₂C(O)NH— or R⁴NH—, wherein
R⁴ is a straight hydrocarbyl group having at least 10 carbon atoms or a tocopherol moiety; and
R³ is selected from the group consisting of H, C₁₋₃ alkyl, —OH, —N₃, C₂₋₆ alkynyl, —COOH, —NH₂, —NHCH₃, —N(CH₃)₂, —NH(CH₂CH₃), —NHC(O)(CH₂)₂COOH, —N(CH₂CH₃)C(O)(CH₂)₂COOH, —N(CH₂CH₃)C(O) CH₃, —C(O)NH₂, —C(O)NHCH₃, —OC(O)(CH₂)₂COOH, and a member of a targeting pair, wherein the C₁₋₃ alkyl group is optionally substituted with one or more (such as one or two) substituents independently selected from the group consisting of —OH, —N₃, C₂₋₆ alkynyl, —COOH, —COOCH₃, —NH₂, —NHCH₃, —N(CH₃)₂, —C(O)NH₂, —C(O)NHCH₃, ₂)₂NH₂, and a member of a targeting pair.

25. (canceled)

26. The composition of claim 1, wherein
(i) a is 1; or
(ii) a is 2.

27.-50. (canceled)

51. The composition of claim 1, wherein
(i) the conjugate of (a) a POX or POZ polymer and (b) one or more hydrophobic chains inhibits aggregation of the LNPs;
(ii) wherein the conjugate of (a) a POX or POZ polymer and (b) one or more hydrophobic chains comprises from about 0.25 mol % to about 50 mol % of the total lipid present in the LNPs;
(iii) the composition is substantially free of a lipid or lipid-like material comprising polyethyleneglycol (PEG); and/or
(iv) the LNPs are non-viral particles.

52.-54. (canceled)

55. The composition of claim 1, wherein
(i) the cationically ionizable lipid comprises a head group which includes at least one nitrogen atom which is capable of being protonated under physiological conditions;
(ii) the cationically ionizable lipid has the structure of Formula (X): 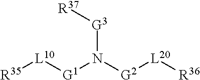
or a pharmaceutically acceptable salt, tautomer, prodrug or stereoisomer thereof, wherein:
one of L¹⁰ and L²⁰ is-O(C═O)—, —(C═O)O—, —C(═O)—, —O—, —S(O)ₓ—, —S—S—, —C(═O)S—, SC(═O)—, NR^{a}C(═O)—, —C(═O)NR^{a}, NR^{a}C(═O)NR^{a}—, —OC(═O)NR^{a}— or —NR^{a}C(═O)O—, and the other of L¹⁰ and L²⁰ is-O(C═O)—, —(C═O)O—, —C(═O)—, —O—, —S(O)ₓ—, —S—S—, —C(═O)S—, SC(═O)—, NR^{a}C(═O)—, —C(═O)NR^{a}—, NR^{a}C(═O)NR^{a}—, —OC(═O)NR^{a}— or —NR^{a}C(═O)O— or a direct bond;
G¹ and G² are each independently unsubstituted C₁-C₁₂ alkylene or C₂₋₁₂ alkenylene;
G³ is C₁₋₂₄ alkylene, C₂₋₂₄ alkenylene, C₃₋₈ cycloalkylene, or C₃₋₈ cycloalkenylene;
R^{a} is H or C₁₋₁₂ alkyl;
R³⁵ and R³⁶ are each independently C₆₋₂₄ alkyl or C₆₋₂₄ alkenyl;
R³⁷ is H, OR⁵⁰, CN, —C(═O)OR⁴⁰, —OC(═O)R⁴⁰ or —NR⁵⁰C(═O)R⁴⁰;
R⁴⁰ is C₁₋₁₂ alkyl;
R⁵⁰ is H or C₁₋₆ alkyl; and
x is 0, 1 or 2;
(iii)
(α) the cationically ionizable lipid is selected from the following structures X-1 to X-36: 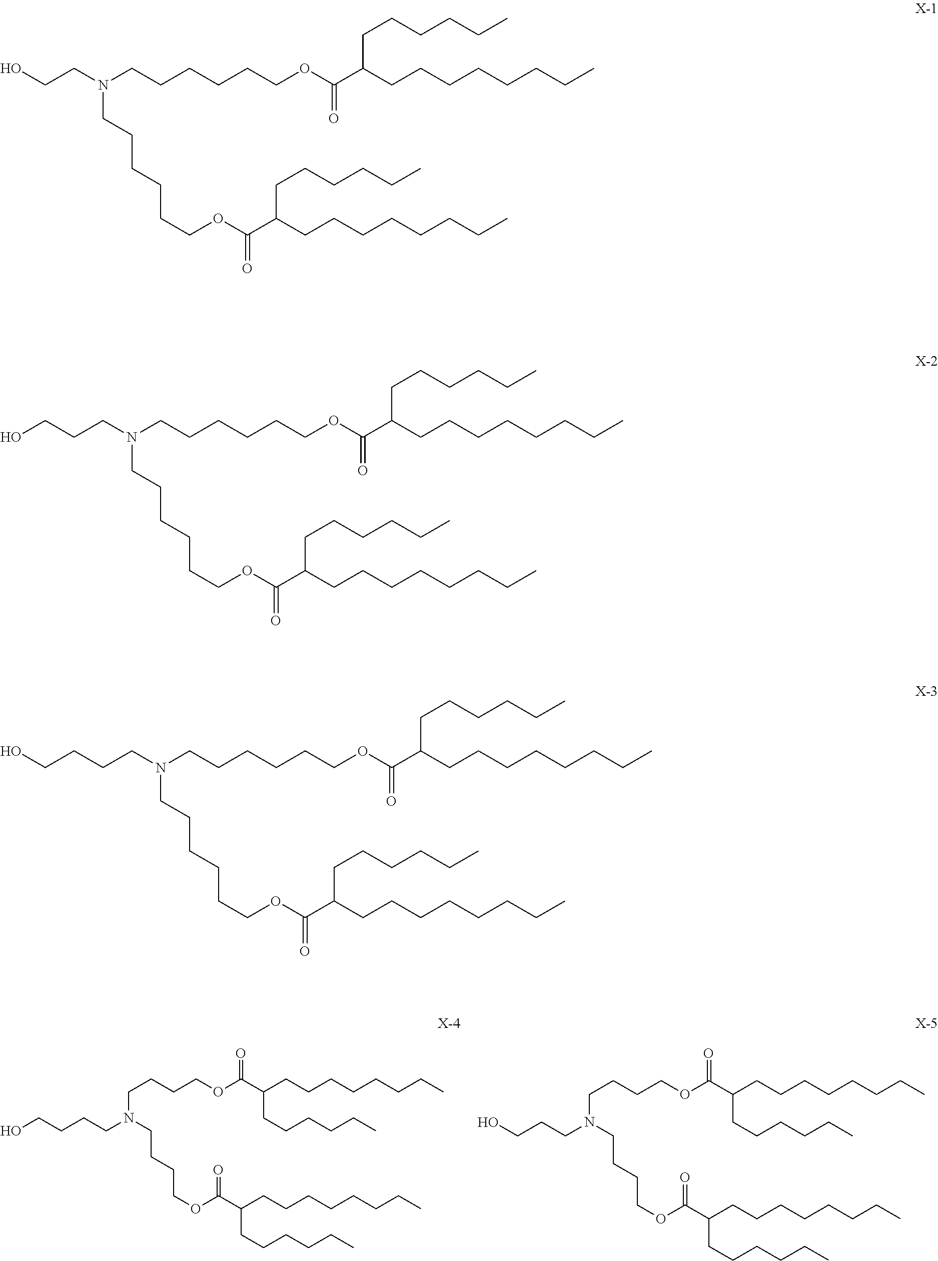 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC ; CCCCCCCCC(CCCCCC)C(=O)OCCCCN(CCCCO)CCCCOC(=O)C(CCCCCC)CCCCCCCC ; CCCCCCCCC(CCCCCC)C(=O)OCCCCN(CCCO)CCCCOC(=O)C(CCCCCC)CCCCCCCC] 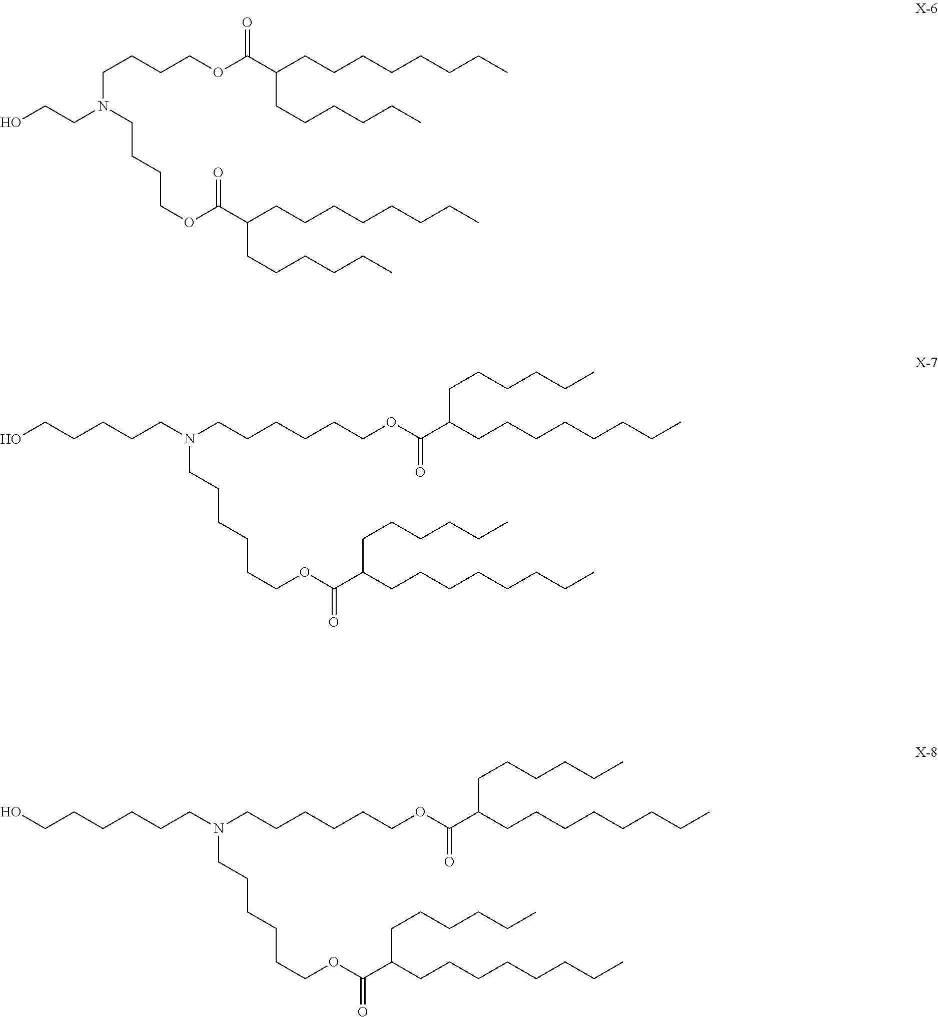 [SMILES: CCCCCCCCC(CCCCCC)C(=O)OCCCCN(CCO)CCCCOC(=O)C(CCCCCC)CCCCCCCC ; CCCCCCCCC(CCCCCC)C(=O)OCCCCCCN(CCCCCO)CCCCCCOC(=O)C(CCCCCC)CCCCCCCC ; CCCCCCCCC(CCCCCC)C(=O)OCCCCCCN(CCCCCCO)CCCCCCOC(=O)C(CCCCCC)CCCCCCCC] 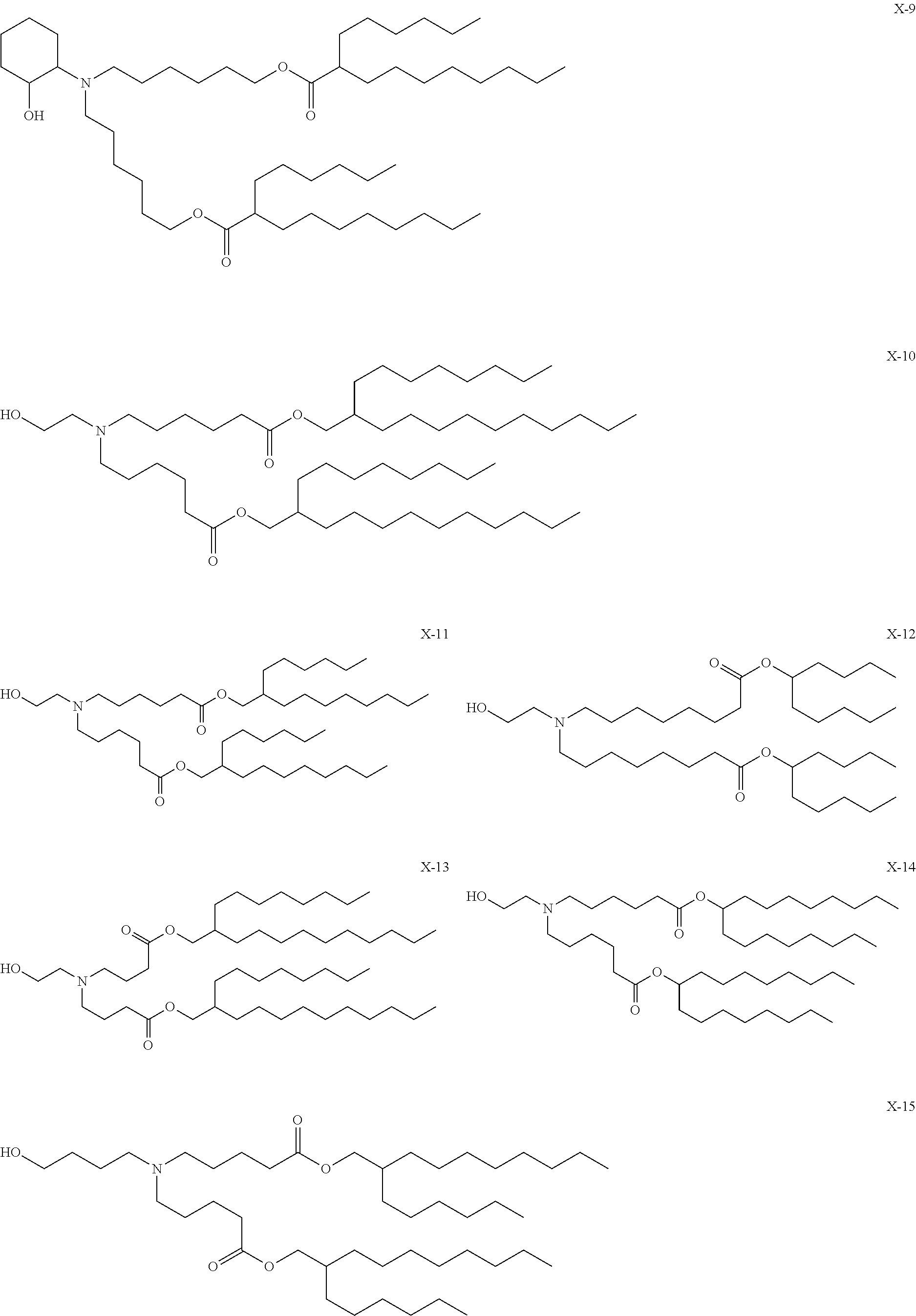 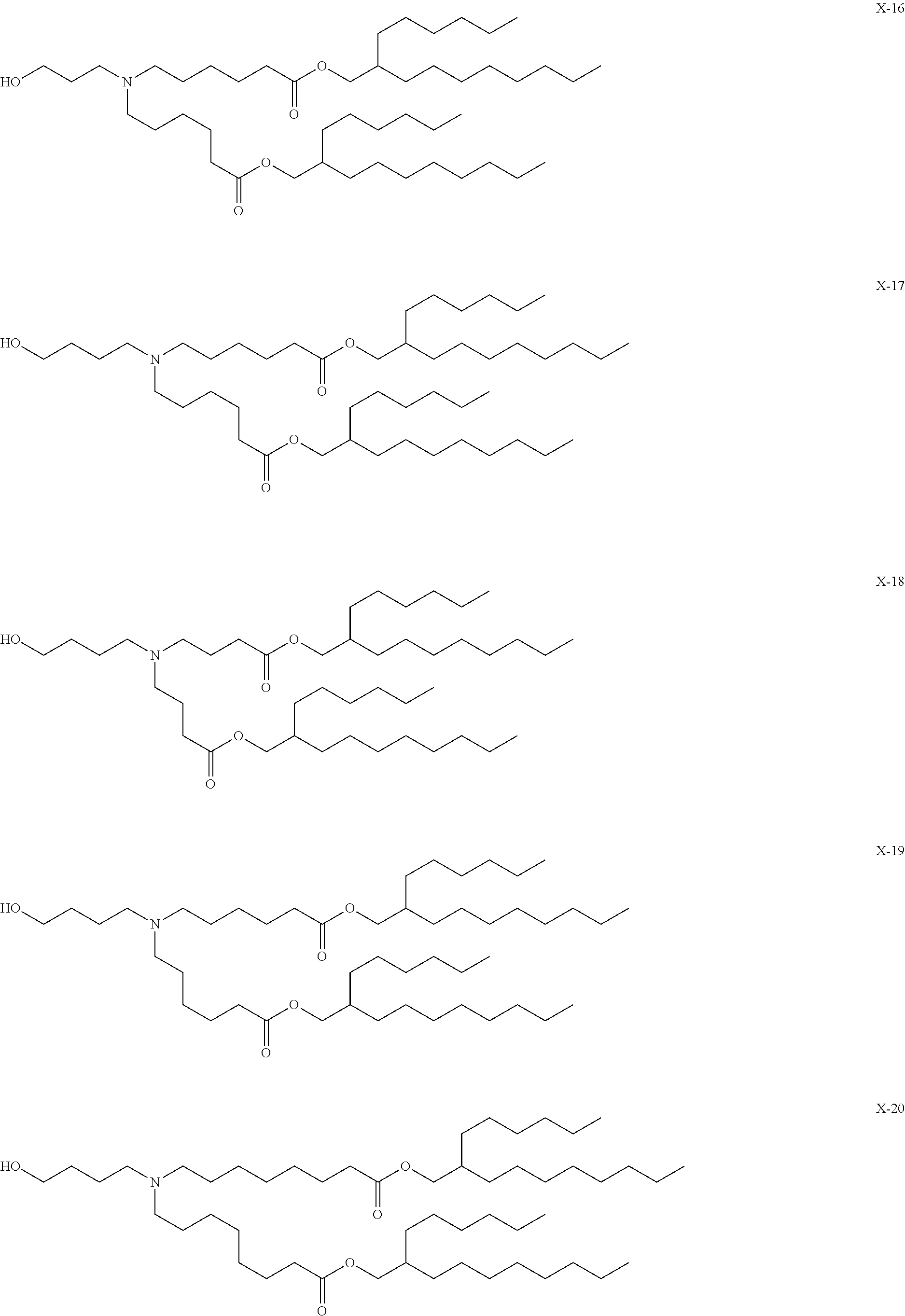 [SMILES: CCCCCCCCC(CCCCCC)COC(=O)CCCCCN(CCCO)CCCCCC(=O)OCC(CCCCCC)CCCCCCCC ; CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCN(CCCCO)CCCC(=O)OCC(CCCCCC)CCCCCCCC ; CCCCCCCCC(CCCCCC)COC(=O)CCCCCN(CCCCO)CCCCCC(=O)OCC(CCCCCC)CCCCCCCC ; CCCCCCCCC(CCCCCC)COC(=O)CCCCCCCN(CCCCO)CCCCCCCC(=O)OCC(CCCCCC)CCCCCCCC] 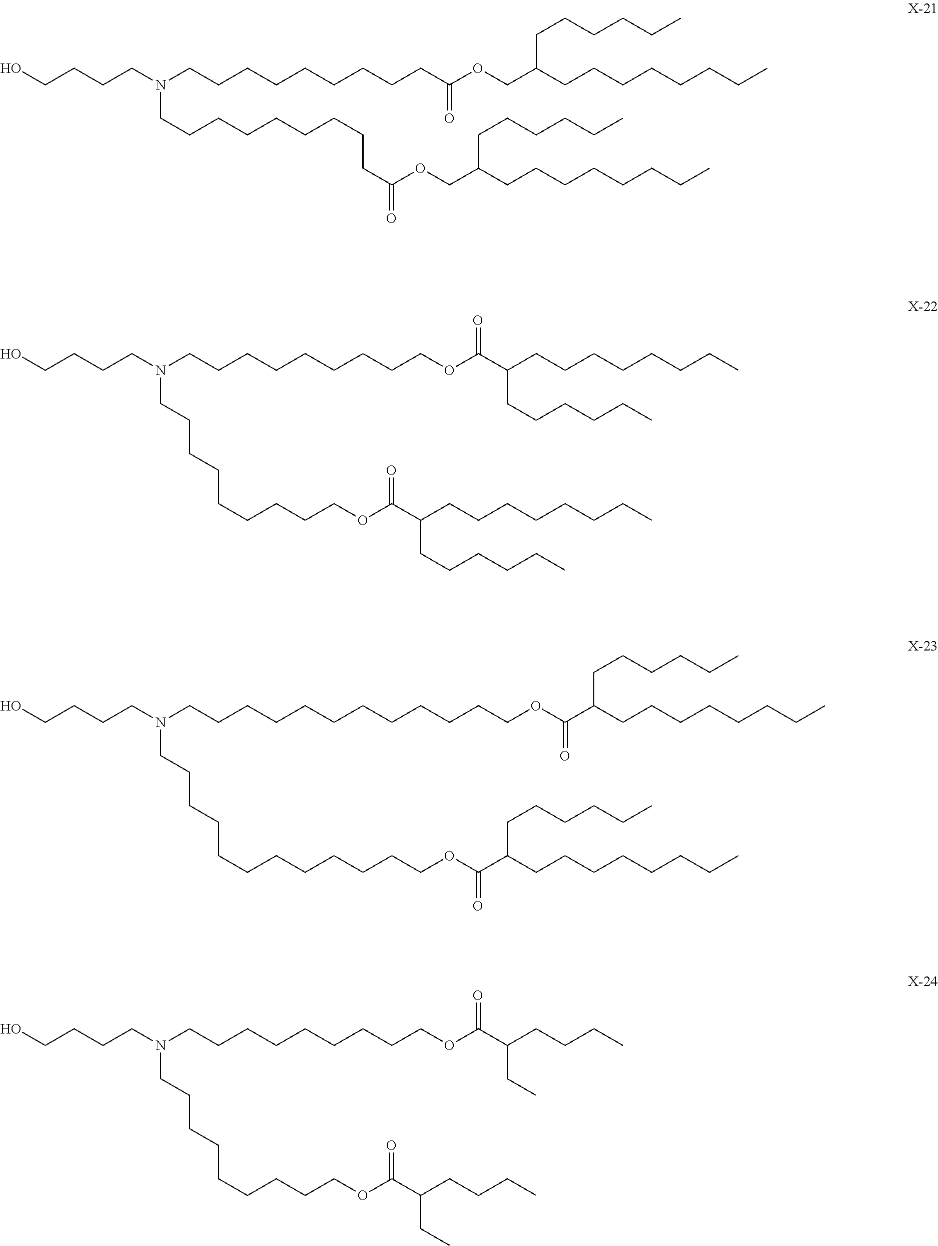 [SMILES: CCCCCCCCC(CCCCCC)COC(=O)CCCCCCCCCN(CCCCO)CCCCCCCCCC(=O)OCC(CCCCCC)CCCCCCCC ; CCCCCCCCC(CCCCCC)C(=O)OCCCCCCCCCN(CCCCO)CCCCCCCCCOC(=O)C(CCCCCC)CCCCCCCC ; CCCCCCCCC(CCCCCC)C(=O)OCCCCCCCCCCCCN(CCCCO)CCCCCCCCCCCCOC(=O)C(CCCCCC)CCCCCCCC ; CCCCC(CC)C(=O)OCCCCCCCCCN(CCCCO)CCCCCCCCCOC(=O)C(CC)CCCC] 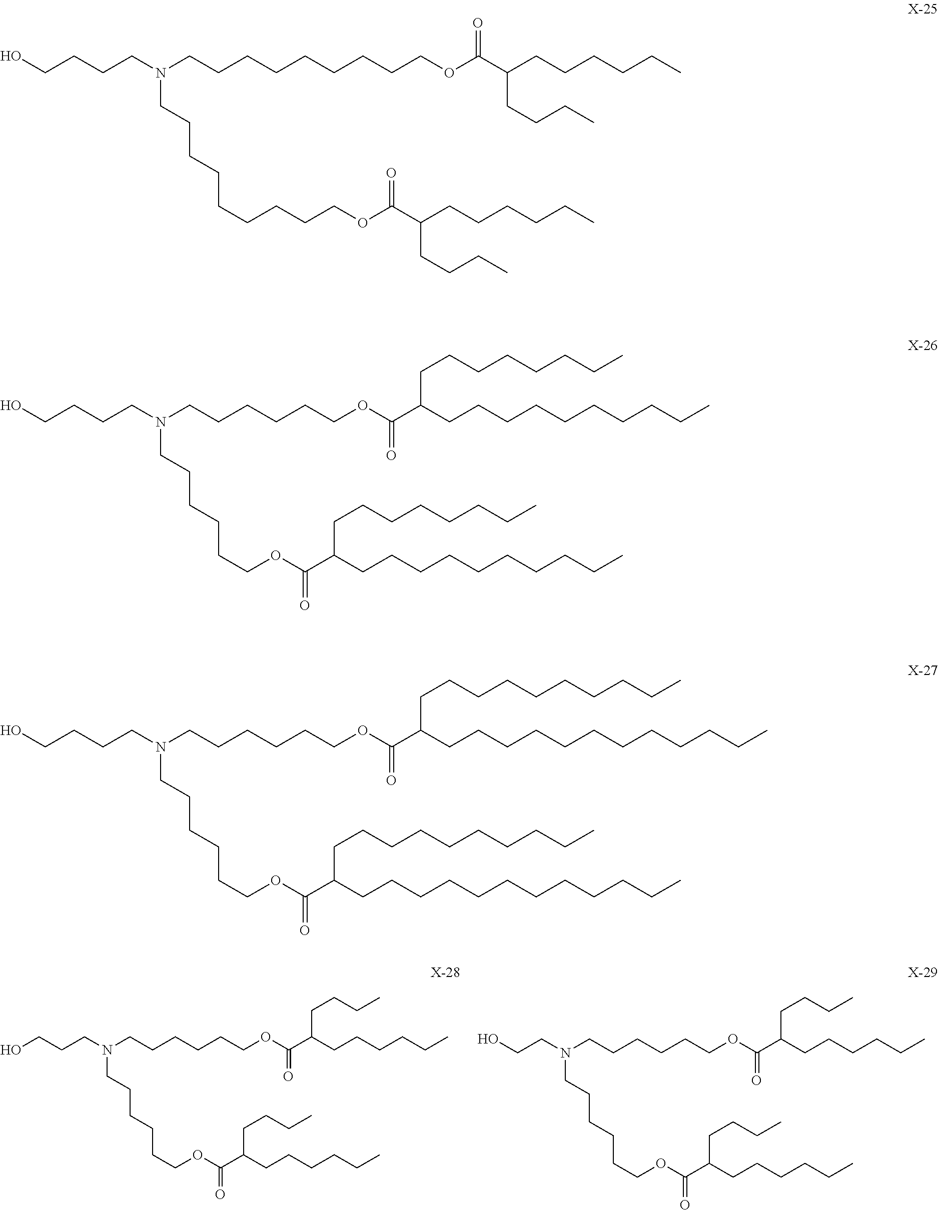 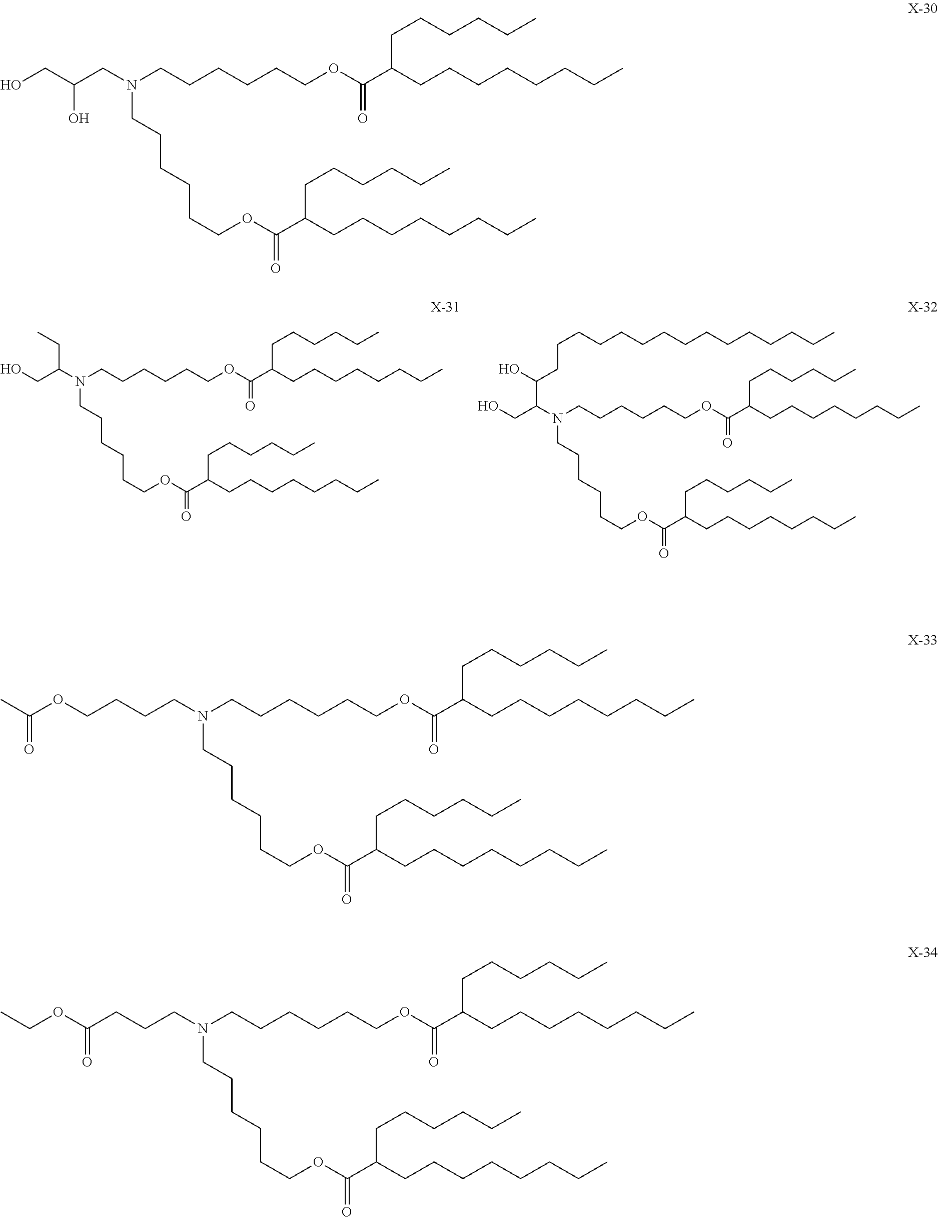 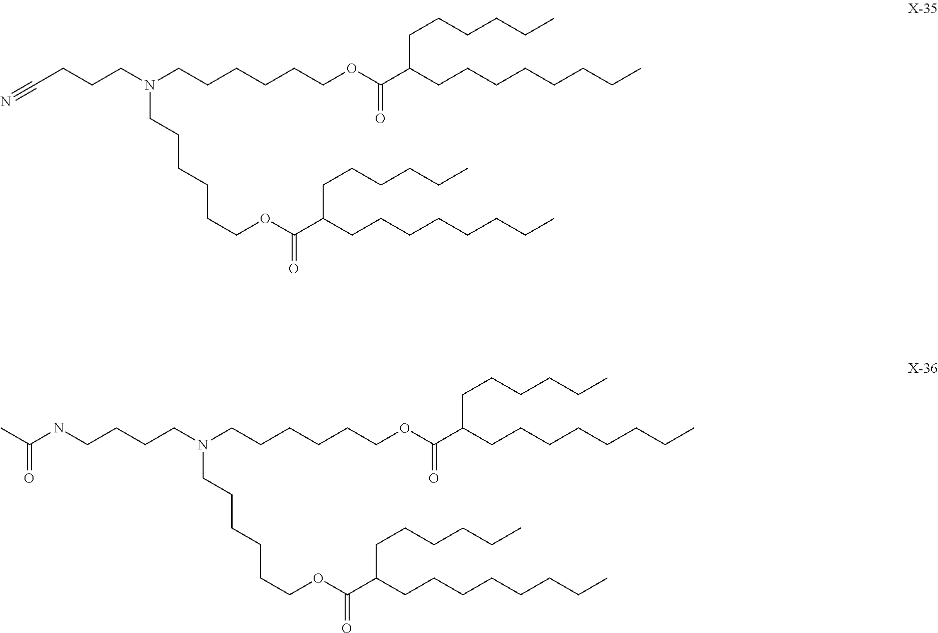
(β) the cationically ionizable lipid is selected from the following structures A to G:

| No. | Structure |
|---|---|
| A | 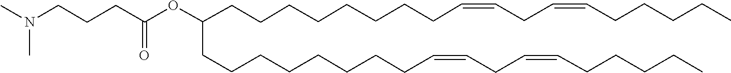 [SMILES: CCCCC/C=C\C/C=C\CCCCCCCCC(CCCCCCCC/C=C\C/C=C\CCCCC)OC(=O)CCCN(C)C] |
| B | 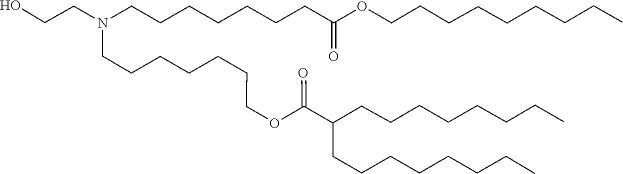 [SMILES: CCCCCCCCCOC(=O)CCCCCCCN(CCO)CCCCCCCOC(=O)C(CCCCCCCC)CCCCCCCC] |
| C | 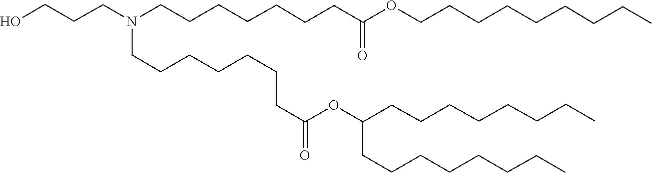 [SMILES: CCCCCCCCCOC(=O)CCCCCCCN(CCCO)CCCCCCCC(=O)OC(CCCCCCCC)CCCCCCCC] |
| D | 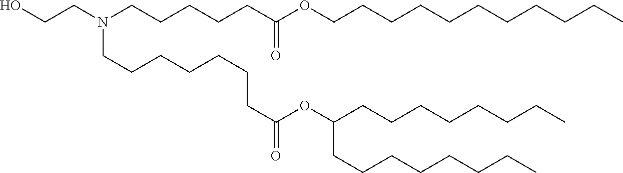 [SMILES: CCCCCCCCCCCOC(=O)CCCCCN(CCO)CCCCCCCC(=O)OC(CCCCCCCC)CCCCCCCC] |
| E | 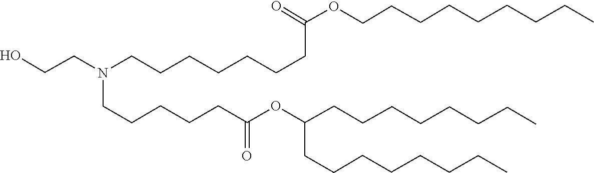 [SMILES: CCCCCCCCCOC(=O)CCCCCCCN(CCO)CCCCCC(=O)OC(CCCCCCCC)CCCCCCCC] |
| F | 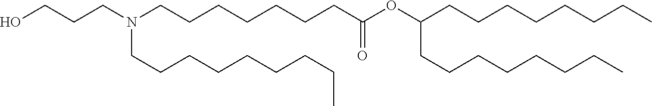 [SMILES: CCCCCCCCCN(CCCO)CCCCCCCC(=O)OC(CCCCCCCC)CCCCCCCC] |
| G | 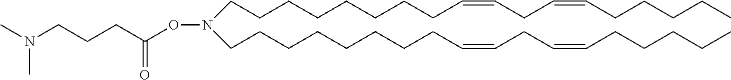 [SMILES: CCCCC/C=C\C/C=C\CCCCCCCCN(CCCCCCCC/C=C\C/C=C\CCCCC)OC(=O)CCCN(C)C] |

or
(γ) the cationically ionizable lipid is the lipid having the structure X-3;
(iv) the cationic or cationically ionizable lipid has the structure of Formula (XI): 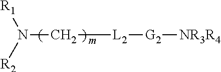
wherein
each of R₁ and R₂ is independently R₅ or -G₁-L₁-R₆, wherein at least one of R₁ and R₂ is -G₁-L₁-R₆;
each of R₅ and R₄ is independently selected from the group consisting of C₁₋₆ alkyl, C₂₋₆ alkenyl, aryl, and C₃₋₁₀ cycloalkyl;
each of R₅ and R₆ is independently a non-cyclic hydrocarbyl group having at least 10 carbon atoms;
each of G₁ and G₂ is independently unsubstituted C₁₋₁₂ alkylene or C₂₋₁₂ alkenylene;
each of L₁ and L₂ is independently selected from the group consisting of —O(C═O)—, —(C═O)O—, —S—S—, —C(═O)S—, —SC(═O)—, —NRaC(═O)—, —C(═O)NRa—, NRaC(═O)NR^{a}—, —OC(═O)NR^{a}— and —NRaC(═O)O—;
Rₐ is H or C₁₋₁₂ alkyl;
m is 0, 1, 2, 3, or 4; and
x is 0, 1 or 2;
(v) the cationically ionizable lipid is selected from the following structures (XIV-1), (XIV-2), and (XIV-3): 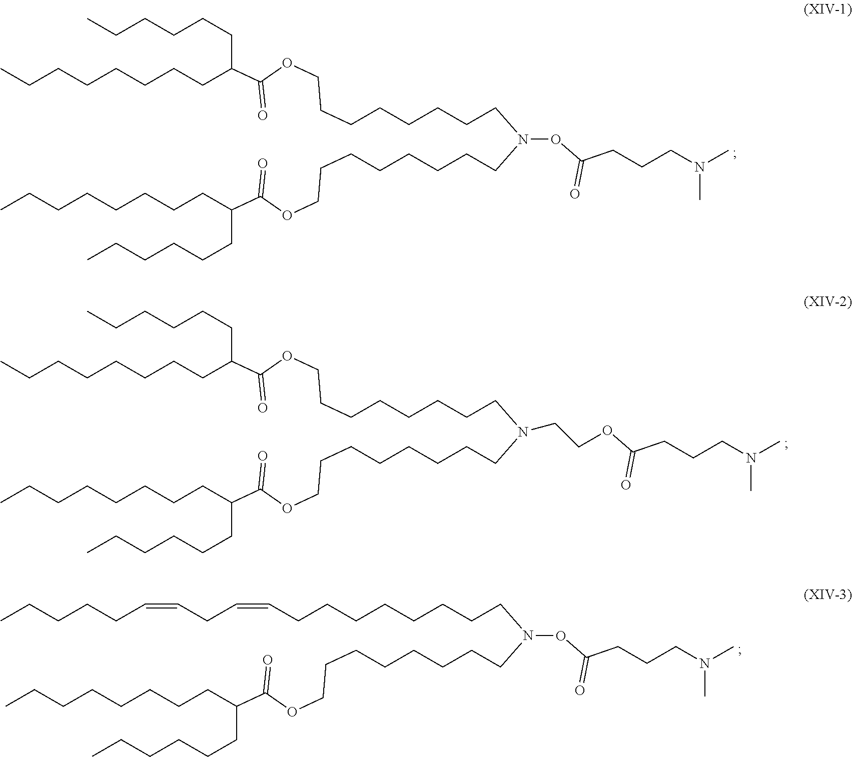
(vi) the cationic or cationically ionizable lipid comprises 2,3-dioleyloxy-1-(N,N-dimethylamino) propane (DODMA), N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), N-(1-(2,3-dioleyloxy) propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), DPL14, or a mixture thereof; or
(vii) the cationic or cationically ionizable lipid comprises from about 20 mol % to about 80 mol % of the total lipid present in the LNPs.

56-61. (canceled)

62. The composition of claim 1, wherein the LNPs further comprise one or more additional lipids, preferably selected from the group consisting of phospholipids, steroids, and combinations thereof, more preferably the LNPs comprise the cationically ionizable lipid, the conjugate, a phospholipid, and a steroid, wherein
i) the phospholipid is selected from the group consisting of phosphatidylcholines, phosphatidylethanolamines, phosphatidylglycerols, phosphatidic acids, phosphatidylserines and sphingomyelins, more preferably selected from the group consisting of distearoylphosphatidylcholine (DSPC), dioleoylphosphatidylcholine (DOPC), dimyristoylphosphatidylcholine (DMPC), dipentadecanoylphosphatidylcholine, dilauroylphosphatidylcholine, dipalmitoylphosphatidylcholine (DPPC), diarachidoylphosphatidylcholine (DAPC), dibehenoylphosphatidylcholine (DBPC), ditricosanoylphosphatidylcholine (DTPC), dilignoceroylphatidylcholine (DLPC), palmitoyloleoyl-phosphatidylcholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C₁₋₆ Lyso PC), dioleoylphosphatidylethanolamine (DOPE), distearoyl-phosphatidylethanolamine (DSPE), dipalmitoyl-phosphatidylethanolamine (DPPE), dimyristoyl-phosphatidylethanolamine (DMPE), dilauroyl-phosphatidylethanolamine (DLPE), and diphytanoyl-phosphatidylethanolamine (DPyPE);
(ii) the phospholipid comprises from about 5 mol % to about 30 mol % of the total lipid present in the LNPs;
(iii) the steroid comprises a sterol;
(iv) the steroid comprises from about 10 mol % to about 60 mol % of the total lipid present in the LNPs; or
(v) the cationic or cationically ionizable lipid comprises from about 20 mol % to about 70 mol % of the total lipid present in the LNPs; the conjugate of (a) a POX or POZ polymer and
(b) one or more hydrophobic chains comprises from about 0.5 mol % to about 15 mol % of the total lipid present in the LNPs; the phospholipid comprises from about 5 mol % to about 25 mol % of the total lipid present in the LNPs; and the steroid comprises from about 25 mol % to about 55 mol % of the total lipid present in the LNPs.

63.-67. (canceled)

68. The composition of claim 1, wherein
(i) the LNPs have a size of from about 30 nm to about 500 nm;
(ii) the RNA is encapsulated within or associated with the LNPs;
(iii) the RNA is mRNA;
(iv) the RNA comprises a modified nucleoside in place of uridine, wherein the modified nucleoside is preferably selected from pseudouridine (w), N₁-methyl-pseudouridine (m1ψ), and 5-methyl-uridine (m5U);
(v) the RNA comprises at least one of the following: a 5′ cap; a 5′ UTR; a 3′ UTR; and a poly-A sequence, and wherein
a. the poly-A sequence comprises at least 100 A nucleotides;
b. the poly-A sequence comprises at least 100 A nucleotides and is an interrupted sequence of A nucleotides;
c. the poly-A sequence is an uninterrupted sequence of A nucleotides; or
d. the 5′ cap is a cap1 or cap2 structure; or
(vi) the RNA encodes one or more peptides or proteins, wherein preferably the one or more peptides or proteins are therapeutic peptides or proteins or comprise an epitope for inducing an immune response against an antigen in a subject.

69-75. (canceled)

76. A method for delivering RNA to cells of a subject, the method comprising administering to a subject the composition of claim 1.

77. (canceled)

78. A method for treating or preventing a disease or disorder in a subject, the method comprising administering to the composition of claim 1, wherein delivering the RNA to cells of the subject is beneficial in treating or preventing the disease or disorder, or
wherein the RNA encodes a therapeutic peptide or protein and wherein delivering the therapeutic peptide or protein to the subject is beneficial in treating or preventing the disease or disorder.

79. (canceled)

80. The method of claim 76, wherein the subject is a mammal.

81. The method of claim 80, wherein the mammal is a human.

82. The method of claim 78, wherein the subject is a mammal.

83. The method of claim 82, wherein the mammal is a human.
